SEQUENCE LISTING (188 sequences)

SEQ ID NO: 1 - RNA, 504 nt, synthetic construct
  source 1..504: mol_type = other RNA
gagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaa

SEQ ID NO: 2 - RNA, 501 nt, synthetic construct
  source 1..501: mol_type = other RNA
caggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggtcgcgttcagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatat

SEQ ID NO: 3 - RNA, 697 nt, synthetic construct
  source 1..697: mol_type = other RNA
cctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaactaagaaacttttgtaaagaaatttactatatatatatgcctctgc

SEQ ID NO: 4 - DNA, 504 bp, synthetic construct
  source 1..504: mol_type = other DNA
gagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaa

SEQ ID NO: 5 - DNA, 2026 bp, synthetic construct
  source 1..2026: mol_type = other DNA
aactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttgactctgactcctagaataa

SEQ ID NO: 6 - DNA, 354 bp, synthetic construct
  source 1..354: mol_type = other DNA
nnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacntnctgccccctccatacanaaaan

SEQ ID NO: 7 - DNA, 267 bp, synthetic construct
  source 1..267: mol_type = other DNA
nnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancacc

SEQ ID NO: 8 - DNA, 299 bp, synthetic construct
  source 1..299: mol_type = other DNA
nnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccnccc

SEQ ID NO: 9 - DNA, 331 bp, synthetic construct
  source 1..331: mol_type = other DNA
nnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacnt

SEQ ID NO: 10 - DNA, 363 bp, synthetic construct
  source 1..363: mol_type = other DNA
nnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacntnctgccccctccatacanaaaanctaagtgtt

SEQ ID NO: 11 - DNA, 394 bp, synthetic construct
  source 1..394: mol_type = other DNA
nnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacntnctgccccctccatacanaaaanctaagtgttggctttaaagtcctaggggccggccccgccg

SEQ ID NO: 12 - DNA, 501 bp, synthetic construct
  source 1..501: mol_type = other DNA
caggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggtcgcgttcagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatat

SEQ ID NO: 13 - DNA, 618 bp, synthetic construct
  source 1..618: mol_type = other DNA
agaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggc

SEQ ID NO: 14 - DNA, 468 bp, synthetic construct
  source 1..468: mol_type = other DNA
nnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaactcttanattatttcttaatacttcccctacacttn

SEQ ID NO: 15 - DNA, 359 bp, synthetic construct
  source 1..359: mol_type = other DNA
nnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagg

SEQ ID NO: 16 - DNA, 397 bp, synthetic construct
  source 1..397: mol_type = other DNA
nnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattg

SEQ ID NO: 17 - DNA, 434 bp, synthetic construct
  source 1..434: mol_type = other DNA
nnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaact

SEQ ID NO: 18 - DNA, 471 bp, synthetic construct
  source 1..471: mol_type = other DNA
nnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaactcttanattatttcttaatacttcccctacacttncta

SEQ ID NO: 19 - DNA, 508 bp, synthetic construct
  source 1..508: mol_type = other DNA
nnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaactcttanattatttcttaatacttcccctacacttnctaagtgttggctttaaagtcctaggggccggccccgccg

SEQ ID NO: 20 - DNA, 855 bp, synthetic construct
  source 1..855: mol_type = other DNA
nnnnnnnannnnnntctcnnncttcagcctcnatacgcgtgtcccggcnggacggtacttcctccnttctggtcctgcccccaggcccgtatagaagcttaaatgcaggagatgaacaatcatgggggaattttgcctcgatggcattgcgttgcanacctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaactaagaaacttttgtaaagaaatttactatatatatatgcctctgc

SEQ ID NO: 21 - DNA, 697 bp, synthetic construct
  source 1..697: mol_type = other DNA
cctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaactaagaaacttttgtaaagaaatttactatatatatatgcctctgc

SEQ ID NO: 22 - DNA, 856 bp, synthetic construct
  source 1..856: mol_type = other DNA
nnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaactaagaaacttttgtaaagaaatttactatatatatatgcctctgc

SEQ ID NO: 23 - DNA, 685 bp, synthetic construct
  source 1..685: mol_type = other DNA
nnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgta

SEQ ID NO: 24 - DNA, 727 bp, synthetic construct
  source 1..727: mol_type = other DNA
nnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgata

SEQ ID NO: 25 - DNA, 770 bp, synthetic construct
  source 1..770: mol_type = other DNA
nnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagtt

SEQ ID NO: 26 - DNA, 813 bp, synthetic construct
  source 1..813: mol_type = other DNA
nnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaacta

SEQ ID NO: 27 - DNA, 813 bp, synthetic construct; identical to SEQ ID NO: 26
  source 1..813: mol_type = other DNA

SEQ ID NO: 28 - DNA, 201 bp, synthetic construct
  source 1..201: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagct

SEQ ID NO: 29 - DNA, 158 bp, synthetic construct
  source 1..158: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctgagcaatcacctatgaacttgctg

SEQ ID NO: 30 - DNA, 741 bp, synthetic construct
  source 1..741: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctgagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaa

SEQ ID NO: 31 - DNA, 504 bp, synthetic construct
  source 1..504: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancacc

SEQ ID NO: 32 - DNA, 536 bp, synthetic construct
  source 1..536: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccnccc

SEQ ID NO: 33 - DNA, 568 bp, synthetic construct
  source 1..568: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacnt

SEQ ID NO: 34 - DNA, 600 bp, synthetic construct
  source 1..600: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacntnctgccccctccatacanaaaanctaagtgtt

SEQ ID NO: 35 - DNA, 631 bp, synthetic construct
  source 1..631: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacntnctgccccctccatacanaaaanctaagtgttggctttaaagtcctaggggccggccccgccg

SEQ ID NO: 36 - DNA, 738 bp, synthetic construct
  source 1..738: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctcaggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggtcgcgttcagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatat

SEQ ID NO: 37 - DNA, 745 bp, synthetic construct
  source 1..745: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaactcttanattatttcttaatacttcccctacacttnctaagtgttggctttaaagtcctaggggccggccccgccg

SEQ ID NO: 38 - DNA, 596 bp, synthetic construct
  source 1..596: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagg

SEQ ID NO: 39 - DNA, 634 bp, synthetic construct
  source 1..634: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattg

SEQ ID NO: 40 - DNA, 671 bp, synthetic construct
  source 1..671: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaact

SEQ ID NO: 41 - DNA, 708 bp, synthetic construct
  source 1..708: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaactcttanattatttcttaatacttcccctacacttncta

SEQ ID NO: 42 - DNA, 745 bp, synthetic construct; identical to SEQ ID NO: 37
  source 1..745: mol_type = other DNA

SEQ ID NO: 43 - DNA, 831 bp, synthetic construct
  source 1..831: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctcctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaactaagaaacttttgtaaagaaatttactatatatatatgcctctgc

SEQ ID NO: 44 - DNA, 819 bp, synthetic construct
  source 1..819: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctnnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgta

SEQ ID NO: 45 - DNA, 861 bp, synthetic construct
  source 1..861: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctnnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgata

SEQ ID NO: 46 - DNA, 904 bp, synthetic construct
  source 1..904: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctnnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagtt

SEQ ID NO: 47 - DNA, 947 bp, synthetic construct
  source 1..947: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctnnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaacta

SEQ ID NO: 48 - DNA, 990 bp, synthetic construct
  source 1..990: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctnnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaactaagaaacttttgtaaagaaatttactatatatatatgcctctgc

SEQ ID NO: 49 - RNA, 10 nt, synthetic construct
  source 1..10: mol_type = other RNA
tttttttttt

SEQ ID NO: 50 - RNA, 10 nt, synthetic construct
  source 1..10: mol_type = other RNA
tcctgccctc

SEQ ID NO: 51 - RNA, 11 nt, synthetic construct
  source 1..11: mol_type = other RNA
cctcctgctta

SEQ ID NO: 52 - RNA, 11 nt, synthetic construct
  source 1..11: mol_type = other RNA
cctcgtaactt

SEQ ID NO: 53 - RNA, 11 nt, synthetic construct
  source 1..11: mol_type = other RNA
cctcctgcctc

SEQ ID NO: 54 - RNA, 12 nt, synthetic construct
  source 1..12: mol_type = other RNA
cctcctgcattt

SEQ ID NO: 55 - RNA, 12 nt, synthetic construct
  source 1..12: mol_type = other RNA
cctcgctgcctc

SEQ ID NO: 56 - RNA, 14 nt, synthetic construct
  source 1..14: mol_type = other RNA
ctgctaagttatct

SEQ ID NO: 57 - RNA, 16 nt, synthetic construct
  source 1..16: mol_type = other RNA
taagttatcctctatt

SEQ ID NO: 58 - RNA, 18 nt, synthetic construct
  source 1..18: mol_type = other RNA
ctgcctctgctaacttat

SEQ ID NO: 59 - DNA, 10 bp, synthetic construct
  source 1..10: mol_type = other DNA
tcctgccctc

SEQ ID NO: 60 - DNA, 11 bp, synthetic construct
  source 1..11: mol_type = other DNA
cctcctgctta

SEQ ID NO: 61 - DNA, 11 bp, synthetic construct
  source 1..11: mol_type = other DNA
cctcgtaactt

SEQ ID NO: 62 - DNA, 11 bp, synthetic construct
  source 1..11: mol_type = other DNA
cctcctgcctc

SEQ ID NO: 63 - DNA, 12 bp, synthetic construct
  source 1..12: mol_type = other DNA
cctcctgcattt

SEQ ID NO: 64 - DNA, 12 bp, synthetic construct
  source 1..12: mol_type = other DNA
cctcgctgcctc

SEQ ID NO: 65 - DNA, 14 bp, synthetic construct
  source 1..14: mol_type = other DNA
  modified_base 12: mod_base = OTHER; note = Uracil
ctgctaagttatct

SEQ ID NO: 66 - DNA, 16 bp, synthetic construct
  source 1..16: mol_type = other DNA
taagttatcctctatt

SEQ ID NO: 67 - DNA, 17 bp, synthetic construct
  source 1..17: mol_type = other DNA
tgcctctgctaacttat

SEQ ID NO: 68 - RNA, 10 nt, synthetic construct
  source 1..10: mol_type = other RNA
aaaaaaaaaa

SEQ ID NO: 69 - DNA, 14 bp, synthetic construct
  source 1..14: mol_type = other DNA
ggacccgcccgagc

SEQ ID NO: 70 - DNA, 15 bp, synthetic construct
  source 1..15: mol_type = other DNA
gggccggccccgccg

SEQ ID NO: 71 - DNA, 22 bp, synthetic construct
  source 1..22: mol_type = other DNA
gcccgcgaggacccgcccgagc

SEQ ID NO: 72 - protein, 385 aa, unidentified
MKLVRKNIEKDNAGQVTLVPEEPEDMWHTYNLVQVGDSLRASTIRKVQTESSTGSVGSNRVRTTLTLCVEAIDFDSQACQLRVKGTNIQENEYVKMGAYHTIELEPNRQFTLAKKQWDSVVLERIEQACDPAWSADVAAVVMQEGLAHICLVTPSMTLTRAKVEVNIPRKRKGNCSQHDRALERFYEQVVQAIQRHIHFDVVKCILVASPGFVREQFCDYLFQQAVKTDNKLLLENRSKFLQVHASSGHKYSLKEALCDPTVASRLSDTKAAGEVKALDDFYKMLQHEPDRAFYGLKQVEKANEAMAIDTLLISDELFRHQDVATRSRYVRLVDSVKENAGTVRIFSSLHVSGEQLSQLTGVAAILRFPVPELSDQEGDSSSEED

SEQ ID NO: 73 - protein, 332 aa, synthetic construct
MKLVRKNIEKDNAGQVTLVPEEPEDMWHTYNLVQVGDSLRASTIRKVQTESSTGSVGSNRVRTTLTLCVEAIDFDSQACQLRVKGTNIQENEYVKMGAYHTIELEPNRQFTLAKKQWDSVVLERIEQACDPAWSADVAAVVMQEGLAHICLVTPSMTLTRAKVEVNIPRKRKGNCSQHDRALERFYEQVVQAIQRHIHFDVVKCILVASPGFVREQFCDYMFQQAVKTDNKLLLENRSKFLQVHASSGHKYSLKEALCDPTVASRLSDTKAAGEVKALDDFYKMLQHEPDRAFYGLKQVEKANEAMAIDTLLISDELFRHQDVATRSRYVRL

SEQ ID NO: 74 - protein, 353 aa, synthetic construct
MKLVRKNIEKDNAGQVTLVPEEPEDMWHTYNLVQVGDSLRASTIRKVQTESSTGSVGSNRVRTTLTLCVEAIDFDSQACQLRVKGTNIQENEYVKMGAYHTIELEPNRQFTLAKKQWDSVVLERIEQACDPAWSADVAAVVMQEGLAHICLVTPSMTLTRAKVEVNIPRKRKGNCSQHDRALERFYEQVVQAIQRHIHFDVVKCILVASPGFVREQFCDYMFQQAVKTDNKLLLENRSKFLQVHASSGHKYSLKEALCDPTVASRLSDTKAAGEVKALDDFYKMLQHEPDRAFYGLKQVEKANEAMAIDTLLISDELFRHQDVATRSRYVRLVDSVKENAGTVRIFSSLHVSG

SEQ ID NO: 75 - protein, 374 aa, synthetic construct
MKLVRKNIEKDNAGQVTLVPEEPEDMWHTYNLVQVGDSLRASTIRKVQTESSTGSVGSNRVRTTLTLCVEAIDFDSQACQLRVKGTNIQENEYVKMGAYHTIELEPNRQFTLAKKQWDSVVLERIEQACDPAWSADVAAVVMQEGLAHICLVTPSMTLTRAKVEVNIPRKRKGNCSQHDRALERFYEQVVQAIQRHIHFDVVKCILVASPGFVREQFCDYMFQQAVKTDNKLLLENRSKFLQVHASSGHKYSLKEALCDPTVASRLSDTKAAGEVKALDDFYKMLQHEPDRAFYGLKQVEKANEAMAIDTLLISDELFRHQDVATRSRYVRLVDSVKENAGTVRIFSSLHVSGEQLSQLTGVAAILRFPVPELS

SEQ ID NO: 76 - protein, 395 aa, synthetic construct
MKLVRKNIEKDNAGQVTLVPEEPEDMWHTYNLVQVGDSLRASTIRKVQTESSTGSVGSNRVRTTLTLCVEAIDFDSQACQLRVKGTNIQENEYVKMGAYHTIELEPNRQFTLAKKQWDSVVLERIEQACDPAWSADVAAVVMQEGLAHICLVTPSMTLTRAKVEVNIPRKRKGNCSQHDRALERFYEQVVQAIQRHIHFDVVKCILVASPGFVREQFCDYMFQQAVKTDNKLLLENRSKFLQVHASSGHKYSLKEALCDPTVASRLSDTKAAGEVKALDDFYKMLQHEPDRAFYGLKQVEKANEAMAIDTLLISDELFRHQDVATRSRYVRLVDSVKENAGTVRIFSSLHVSGEQLSQLTGVAAILRFPVPELSDQEGDSSSEEDTRTRPLEQKL

SEQ ID NO: 77 - protein, 416 aa, synthetic construct
MKLVRKNIEKDNAGQVTLVPEEPEDMWHTYNLVQVGDSLRASTIRKVQTESSTGSVGSNRVRTTLTLCVEAIDFDSQACQLRVKGTNIQENEYVKMGAYHTIELEPNRQFTLAKKQWDSVVLERIEQACDPAWSADVAAVVMQEGLAHICLVTPSMTLTRAKVEVNIPRKRKGNCSQHDRALERFYEQVVQAIQRHIHFDVVKCILVASPGFVREQFCDYMFQQAVKTDNKLLLENRSKFLQVHASSGHKYSLKEALCDPTVASRLSDTKAAGEVKALDDFYKMLQHEPDRAFYGLKQVEKANEAMAIDTLLISDELFRHQDVATRSRYVRLVDSVKENAGTVRIFSSLHVSGEQLSQLTGVAAILRFPVPELSDQEGDSSSEEDTRTRPLEQKLISEEDLAANDILDYKDDDDKV

SEQ ID NO: 78 - protein, 416 aa, synthetic construct; identical to SEQ ID NO: 77

SEQ ID NO: 79 - protein, 245 aa, unidentified
MAGLELLSDQGYRVDGRRAGELRKIQARMGVFAQADGSAYIEQGNTKALAVVYGPHEIRGSRARALPDRALVNCQYSSATFSTGERKRRPHGDRKSCEMGLQLRQTFEAAILTQLHPRSQIDIYVQVLQADGGTYAACVNAATLAVLDAGIPMRDFVCACSAGFVDGTALADLSHVEEAAGGPQLALALLPASGQIALLEMDARLHEDHLERVLEAAAQAARDVHTLLDRVVRQHVREASILLGD

SEQ ID NO: 80 - protein, 221 aa, synthetic construct
MAGLELLSDQGYRVDGRRAGELRKIQARMGVFAQADGSAYIEQGNTKALAVVYGPHEIRGSRARALPDRALVNCQYSSATFSTGERKRRPHGDRKSCEMGLQLRQTFEAAILTQLHPRSQIDIYVQVLQADGGTYAACVNAATLAVLDAGIPMRDFVCACSAGFVDGTALADLSHVEEAAGGPQLALALLPASGQIALLEMDARLHEDHLERVLEAAAQAA

SEQ ID NO: 81 - protein, 235 aa, synthetic construct
MAGLELLSDQGYRVDGRRAGELRKIQARMGVFAQADGSAYIEQGNTKALAVVYGPHEIRGSRARALPDRALVNCQYSSATFSTGERKRRPHGDRKSCEMGLQLRQTFEAAILTQLHPRSQIDIYVQVLQADGGTYAACVNAATLAVLDAGIPMRDFVCACSAGFVDGTALADLSHVEEAAGGPQLALALLPASGQIALLEMDARLHEDHLERVLEAAAQAARDVHTLLDRVVRQH

SEQ ID NO: 82 - protein, 248 aa, synthetic construct
MAGLELLSDQGYRVDGRRAGELRKIQARMGVFAQADGSAYIEQGNTKALAVVYGPHEIRGSRARALPDRALVNCQYSSATFSTGERKRRPHGDRKSCEMGLQLRQTFEAAILTQLHPRSQIDIYVQVLQADGGTYAACVNAATLAVLDAGIPMRDFVCACSAGFVDGTALADLSHVEEAAGGPQLALALLPASGQIALLEMDARLHEDHLERVLEAAAQAARDVHTLLDRVVRQHVREASILLGDTRT

SEQ ID NO: 83 - protein, 262 aa, synthetic construct
MAGLELLSDQGYRVDGRRAGELRKIQARMGVFAQADGSAYIEQGNTKALAVVYGPHEIRGSRARALPDRALVNCQYSSATFSTGERKRRPHGDRKSCEMGLQLRQTFEAAILTQLHPRSQIDIYVQVLQADGGTYAACVNAATLAVLDAGIPMRDFVCACSAGFVDGTALADLSHVEEAAGGPQLALALLPASGQIALLEMDARLHEDHLERVLEAAAQAARDVHTLLDRVVRQHVREASILLGDTRTRPLEQKLISEEDLA

SEQ ID NO: 84 - protein, 276 aa, synthetic construct
MAGLELLSDQGYRVDGRRAGELRKIQARMGVFAQADGSAYIEQGNTKALAVVYGPHEIRGSRARALPDRALVNCQYSSATFSTGERKRRPHGDRKSCEMGLQLRQTFEAAILTQLHPRSQIDIYVQVLQADGGTYAACVNAATLAVLDAGIPMRDFVCACSAGFVDGTALADLSHVEEAAGGPQLALALLPASGQIALLEMDARLHEDHLERVLEAAAQAARDVHTLLDRVVRQHVREASILLGDTRTRPLEQKLISEEDLAANDILDYKDDDDKV

SEQ ID NO: 85 - protein, 354 aa, unidentified
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGHGRFQTMEEKKAFMGPLKKDRIAKEEGA

SEQ ID NO: 86 - protein, 308 aa, synthetic construct
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKR

SEQ ID NO: 87 - protein, 327 aa, synthetic construct
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGH

SEQ ID NO: 88 - protein, 346 aa, synthetic construct
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGHGRFQTMEEKKAFMGPLKKD

SEQ ID NO: 89 - protein, 366 aa, synthetic construct
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGHGRFQTMEEKKAFMGPLKKDRIAKEEGATRTRPLEQKLIS

SEQ ID NO: 90 - protein, 385 aa, synthetic construct
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGHGRFQTMEEKKAFMGPLKKDRIAKEEGATRTRPLEQKLISEEDLAANDILDYKDDDDKV

SEQ ID NO: 91 - RNA, 132 nt, synthetic construct
  source 1..132: mol_type = other RNA
gagatggtgaccgagctgctgggaggagacatggtgaaccagagtttcatctgcgacccggacgacgagaccttcatcaaaaacatcatcatccaggactgtatgtggagcggcttctcggccgccgccaag

SEQ ID NO: 92 - RNA, 4365 nt, synthetic construct
  source 1..4365: mol_type = other RNA
ctggatttttttcgggtagtggaaaaccaggtaagcaccgaagtccacttgccttttaatttatttttttatcactttaatgctgagatgagtcgaatgcctaaatagggtgtcttttctcccattcctgcgctattgacacttttctcagagtagttatggtaactggggctggggtggggggtaatccagaactggatcggggtaaagtgacttgtcaagatgggagaggagaaggcagagggaaaacgggaatggtttttaagactaccctttcgagatttctgccttatgaatatattcacgctgactcccggccggtcggacattcctgctttattgtgttaattgctctctgggttttggggggctgggggttgctttgcggtgggcagaaagccccttgcatcctgagctccttggagtagggaccgcatatcgcctgtgtgagccagatcgctccgcagccgctgacttgtccccgtctccgggagggcatttaaatttcggctcaccgcatttctgacagccggagacggacactgcggcgcgtcccgcccgcctgtccccgcggcgattccaacccgccctgatccttttaagaagttggcatttggctttttaaaaagcaataatacaatttaaaacctgggtctctagaggtgttaggacgtggtgttgggtaggcgcaggcaggggaaaagggaggcgaggatgtgtccgattctcctggaatcgttgacttggaaaaaccagggcgaatctccgcacccagccctgactcccctgccgcggccgccctcgggtgtcctcgcgcccgagatgcggaggaactgcgaggagcggggctctgggcggttccagaacagctgctacccttggtggggtggctccgggggaggtatcgcagcggggtctctggcgcagttgcatctccgtattgagtgcgaagggaggtgcccctattattatttgacaccccccttgtatttatggaggggtgttaaagcccgcggctgagctcgccactccagccggcgagagaaagaagaaaagctggcaaaaggagtgttggacgggggcggtactgggggtggggacgggggcggtggagagggaaggttgggaggggctgcggtgccggcgggggtaggagagcggctagggcgcgagtgggaacagccgcagcggaggggccccggcgcggagcggggttcacgcagccgctagcgcccaggcgcctctcgccttctccttcaggtggcgcaaaactttgtgccttggattttggcaaattgttttcctcaccgccacctcccgcggcttcttaagggcgccagggccgatttcgattcctctgccgctgcggggccgactcccgggctttgcgctccgggctcccgggggagcgggggctcggcgggcaccaagccgctggttcactaagtgcgtctccgagatagcaggggactgtccaaagggggtgaaagggtgctccctttattcccccaccaagaccacccagccgctttaggggatagctctgcaaggggagaggttcgggactgtggcgcgcactgcgcgctgcgccaggtttccgcaccaagacccctttaactcaagactgcctcccgctttgtgtgccccgctccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctggtaagcgaagcccgcccaggcctgtcaaaagtgggcggctggatacctttcccattttcattggcagcttatttaacgggccactcttattaggaaggagagatagcagatctggagagatttgggagctcatcacctctgaaaccttgggctttagcgtttcctcccatcccttccccttagactgcccatgtttgcagcccccctccccgtttgtctcccacccctcaggaatttcatttaggtttttaaaccttctggcttatcttacaactcaatccacttcttcttacctcccgttaacattttaattgccctggggcggggtggcagggagtgtatgaatgaggataagagaggattgatctctgagagtgaatgaattgcttccctcttaacttccgagaagtggtgggatttaatgaactatctacaaaaatgaggggctgtgtttagaggctaggcagggcctgcctgagtgcgggagccagtgaactgcctcaagagtgggtgggctgaggagctgggatcttctcagcctattttgaacactgaaaagcaaatccttgccaaagttggacttttttttttcttttattccttcccccgccctcttggacttttggcaaaactgcaatttttttttttttatttttcatttccagtaaaatagggagttgctaaagtcataccaagcaatttgcagctatcatttgcaacacctgaagtgttcttggtaaagtccctcaaaaataggaggtgcttgggaatgtgctttgctttgggtgtgtccaaagcctcattaagtcttaggtaagaattggcatcaatgtcctatcctgggaagttgcacttttcttgtccatgccataacccagctgtctttccctttatgagactcttaccttcatggtgagaggagtaagggtggctggctagattggttctttttttttttttttccttttttaagacggagtctcactctgtcactaggctggagtgcagtggcgcaatcaacctccaaccccctggttcaagagattctcctgcctcagcctcccaagtagctgggactacaggtgcacaccaccatgccaggctaatttttgtaattttagtagagatggggtttcatcgtgttggccaggatggtctctcctgacctcacgatccgcccacctcggcctcccaaagtgctgggattacaggtgtgagccagggcaccaggcttagatgtggctctttggggagataattttgtccagagacctttctaacgtattcatgccttgtatttgtacagcattaatctggtaattgattattttaatgtaaccttgctaaaggagtgatttctatttcctttcttaaagaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 93 - RNA, 3425 nt, synthetic construct
  source 1..3425: mol_type = other RNA
agtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggtgaggcccgtgggcaacccagccaggccctgcctccagctgggctgagccctctgtttacaggtgggtggcagaagaaggtgccctgcccttctgtttcctctcttgttgtggtttctcaaccaggaagtcctttctaacatctaacccccattcattttactgcagaatcagttgactctctctataacgtggctggccgaggtcatgtctggatgggatgcgtctgtgtttccgctaaatcttgtgctctcttgccagcatgatcatgtcccctgtccacctgctccagccactatccctctcccacttacagcagaagaaagggctggtgagaaaggtggattacaggcccacttctgccactgacgagccctatgaatgtggcctacacccccttagcttcactgggtctcagtttccctatctgtatattgggagcagttgtgaagctcagaagagaaatgtctgtgaaaaggttatgaacaggagggagagtggaaaccaacctgctggatcgtgtccacagaccctggaatggggccacatgcttggtttgtcaaattgcagacgccggccgggtgcgatggctcatgcctgtaatcccagcactttgggaggccgaggcggacagatcacttgaggtcgggagttcgagaccagcctgaccaacatggagaaaccccgtctctactgaaaatacaaaattagccaggcatggtggcacatgcctataatcccagctacttgggaaggctgaggcaggagaatcacttgaacctgggagacggaggttgtggtgagcctagatcgtgccattgtactccagcctgggcaacaagagtgaaactccgtctcaaaaaaaaaaaatttgcagacgccatcccatccaggcctttgctttcactgatgaagaaactgagatacagagagggcagggcacctgttcggagtttatgaaatgcccccccaccattatctttcttgatcatataagaatctggtgaggcaaggtagggcgtgatctttatctctattttatcgttttatttaagcgggaacaggactgctcagtggctgggggccttgcccaagatctccaagtactggggaaccccagggaggccctggggggtggcagtgttcctatttcagccccactctgcttccccctcccaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacaggtggccttcaccgtcattgaaaccttctcttggttattcagagctgaccagggccactgctaaccagggggaggctttgtgtgcattagaaatggtgtcccttctgggcagacgcaggcagagcccgggaagacgccctcagaagattggaaaaagattccccttcttcctgggaagttgtagcttgcgtcagcacatataattcaatcgtgagaatgcaggctgggtttttgcccccacttggctgagtgaagtgtacagtgaacaacctatgtaactatttgctggccctggagccgactctgccccagagtctgggtgccaggtgctttgcccgcatggcccatttcagtcacgctgcagtcctgtcaggaaaaaatcagtgttattctcattctacatatgagaaaactgaggcttgcagatataagggccaaaagttacacagctagtgagtgatggggctgagtttcagactccacagtctcttaaccaccaagcagcatgcccagagtagaggtgagaaggaaggagagagctgcggtccacatgagcatctggacctagcatggacaactcactcctccctggctctcgctttgttcttgttgcgggtgtggtggtggtgggactcaaagacggtaaagatagctttctctcctccctggggaatctgggggttgtttaaaaggcctgctcctcttttagaaggcaggagggccccaagggaagcagaaggtgacagaaggggaaagggtcctctgatcattgctcaccccacagagatcttgaaaggaggggtcttgatccagcggaacccccagctctgctaccaggacacgattttgtggaaggacatcttccacaagaacaaccagctggctctcacactgatagacaccaaccgctctcgggcctgtaagccatgcccctccctgctgcctcttctctcagacagcctgaccccagccgcaaactcccaacttacaacccagtgcctgcccgccactgccccagccgcctacaccacccatttcctccctctctgtccctcctgccatctccctgtgcctcttcatctctggggttctctgtcttgtctccctctgcttataggttgtgcctctggtttgggggcctctcagcctgtctgggtccctcccttgctgtgcagttggcctcgtggcctctgctgctgtttgtgcctctctctgttactaacccgtcctctcgctgttagacatctctctcactgcctgtctctggttctgtcctcaggccacccctgttctccgatgtgtaagggctcccgctgctggggagagagttctgaggattgtcagagccgtgagtctcagggaggcctggagtcagggaaggggagggctggggccgggtggaatgcaggtgtcatacaggtgacatgggaggggtgggataacaggcttgggatgtctcccctgggccaggtagtctccctagaaggtgatgctgatgagggtctggtgcccagggcgccactcagccctcatcctgccctttgcccaacagtgacgcgcactgtctgtgccggtggctgtgcccgctgcaaggggccactgcccactgactgctgccatgagcagtgtgctgccggctgcacgggccccaagcactctgactgcctggtatgtgcctctgctttgtgcccaatgtgctctaccccccaggatgcaaggggtgggcaccctgcctggtactgccctattgcccctggcacaccagggcaaaacagcacagtgaaagccagccacctgtccccccaggcctgcctccacttcaaccacagtggcatctgtgagctgcactgcccagccctggtcacctacaacacagaca

SEQ ID NO: 94 - RNA, 2022 nt, synthetic construct
  source 1..2022: mol_type = other RNA
gccccatccctgcctgtggctaagagcaccctcctgcagagggtgggaaggagagatgagtccagtatgccaggcccctcacggaaggctgcatgctgggctggggaggggccaccatcctgcctctccttcctccacagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccagggtcagtgccctcggtcacactgtgtggctgtctgcttacctcccccaaccccggtggactagggtccctttctctgatgttccctcaactgtcacctctcaaggaaaccccattatccctacaaaaaattcttactgccttccaacccctgtgaccccattctctccacggtgactgtgtcataccccaaaggtgacctctgtttttctcctgtgaccctgtcaccttccatggagtccccatcccagatccgtgagtgacccccatcatgactttctttcttgtccccagagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctggtatggagtccagtctaagcagagagactgatgggcaggggaggtgggaccttcagcccagggtccactgtgggggcagagggagtggcagagacaccggggttccttcccctaatgggtcaccttctcttgacctttcagaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggcaccggcctatgtctgggggtggctctgtgcc

SEQ ID NO: 95 - DNA, 20 bp, synthetic construct
  source 1..20: mol_type = other DNA
ccccggttctggatctggta

SEQ ID NO: 96 - DNA, 20 bp, synthetic construct
  source 1..20: mol_type = other DNA
gttctcatgaagccacggga

SEQ ID NO: 97 - DNA, 21 bp, synthetic construct
  source 1..21: mol_type = other DNA
gtgcgggagtatgagttaaga

SEQ ID NO: 98 - DNA, 21 bp, synthetic construct
  source 1..21: mol_type = other DNA
acccagatcgatgtgttcctg

SEQ ID NO: 99 - DNA, 20 bp, synthetic construct
  source 1..20: mol_type = other DNA
cgagctggtaacaaagcagc

SEQ ID NO: 100 - DNA, 20 bp, synthetic construct
  source 1..20: mol_type = other DNA
ccatacctctcttgagggcg

SEQ ID NO: 101 - DNA, 20 bp, synthetic construct
  source 1..20: mol_type = other DNA
aagtgcccttagaaaccggg

SEQ ID NO: 102 - DNA, 20 bp, synthetic construct
  source 1..20: mol_type = other DNA
gcaggcttgagagtcgaagt

SEQ ID NO: 103 - DNA, 20 bp, synthetic construct
  source 1..20: mol_type = other DNA
ggtggtgaactacagcgagt

SEQ ID NO: 104 - DNA, 20 bp, synthetic construct
  source 1..20: mol_type = other DNA
acaactcgagctgccatcag

SEQ ID NO: 105 - DNA, 20 bp, synthetic construct
  source 1..20: mol_type = other DNA
tccagttccgccttcacatc

SEQ ID NO: 106 - DNA, 20 bp, synthetic construct
  source 1..20: mol_type = other DNA
cacacctcaaaaaggaccgc

SEQ ID NO: 107 - DNA, 20 bp, synthetic construct
  source 1..20: mol_type = other DNA
ccaggactgaaaggcggttt

SEQ ID NO: 108 - DNA, 20 bp, synthetic construct
  source 1..20: mol_type = other DNA
accagatttcacgagtggca

SEQ ID NO: 109 - DNA, 20 bp, synthetic construct
  source 1..20: mol_type = other DNA
tggatagtcccctgaaccga

SEQ ID NO: 110 - DNA, 20 bp, synthetic construct
  source 1..20: mol_type = other DNA
gccacaaaagcggtcaaagg

SEQ ID NO: 111 - DNA, 17 bp, synthetic construct
  source 1..17: mol_type = other DNA
tcggaccagggctaccg

SEQ ID NO: 112 - DNA, 20 bp, synthetic construct
  source 1..20: mol_type = other DNA
tagaccacagccagtgcctt

SEQ ID NO: 113 - DNA, 20 bp, synthetic construct
  source 1..20: mol_type = other DNA
cagtcttgaaggtccggagg

SEQ ID NO: 114 - DNA, 20 bp, synthetic construct
  source 1..20: mol_type = other DNA
caagaaggaggtggtggagg

SEQ ID NO: 115 - DNA, 20 bp, synthetic construct
  source 1..20: mol_type = other DNA
gcgcaagttggtgtgaacaa

SEQ ID NO: 116 - DNA, 23 bp, synthetic construct
  source 1..23: mol_type = other DNA
gtctttttctttaggcgtgtgct

SEQ ID NO: 117 - DNA, 20 bp, synthetic construct
  source 1..20: mol_type = other DNA
ccattaacgacgacggcaac

SEQ ID NO: 118 - DNA, 20 bp, synthetic construct
  source 1..20: mol_type = other DNA
agcagcagtagcagcaagaa

SEQ ID NO: 119 - DNA, 20 bp, synthetic construct
  source 1..20: mol_type = other DNA
acctttggggttgaaggtga

SEQ ID NO: 120 - DNA, 20 bp, synthetic construct
  source 1..20: mol_type = other DNA
aggtgacacgagcagtgatg

SEQ ID NO: 121 - DNA, 20 bp, synthetic construct
  source 1..20: mol_type = other DNA
aaccatggccaactgtgagc

SEQ ID NO: 122 - DNA, 20 bp, synthetic construct
  source 1..20: mol_type = other DNA
cagaccaacaaggcggaatc

SEQ ID NO: 123 - DNA, 20 bp, synthetic construct
  source 1..20: mol_type = other DNA
acgtcatctgggatgtggac

SEQ ID NO: 124 - DNA, 20 bp, synthetic construct
  source 1..20: mol_type = other DNA
agctggtgtggaagaatggc

SEQ ID NO: 125 - DNA, 20 bp, synthetic construct
  source 1..20: mol_type = other DNA
aaaggcacacctgtcaactg

SEQ ID NO: 126 - DNA, 21 bp, synthetic construct
  source 1..21: mol_type = other DNA
ttctcctttgcctggaagtga

SEQ ID NO: 127 - DNA, 22 bp, synthetic construct
  source 1..22: mol_type = other DNA
gatagcttgttctcgttgagcc

SEQ ID NO: 128 - DNA, 20 bp, synthetic construct
  source 1..20: mol_type = other DNA
aaagaaaagaacccgccgag

SEQ ID NO: 129 - DNA, 20 bp, synthetic construct
  source 1..20: mol_type = other DNA
aacatgcctgctcgtacagt

SEQ ID NO: 130 - DNA, 20 bp, synthetic construct
  source 1..20: mol_type = other DNA
tgccatctgcacatactccc

SEQ ID NO: 131 - DNA, 21 bp, synthetic construct
  source 1..21: mol_type = other DNA
catcatgggccagaatgttgc

SEQ ID NO: 132 - DNA, 22 bp, synthetic construct
  source 1..22: mol_type = other DNA
taccatgtctggagttacgctg

SEQ ID NO: 133 - DNA, 21 bp, synthetic construct
  source 1..21: mol_type = other DNA
gatgtaggatccagcacgagg

SEQ ID NO: 134 - DNA, 20 bp, synthetic construct
  source 1..20: mol_type = other DNA
ctgttgtctgaatgccgcct

SEQ ID NO: 135 - DNA, 20 bp, synthetic construct
  source 1..20: mol_type = other DNA
cctcagtctcacagaccacc

SEQ ID NO: 136 - DNA, 21 bp, synthetic construct
  source 1..21: mol_type = other DNA
ttctcccccaggttaccctta

SEQ ID NO: 137 - DNA, 20 bp, synthetic construct
  source 1..20: mol_type = other DNA
acagtgagggtttcaggtgg

SEQ ID NO: 138 - DNA, 19 bp, synthetic construct
  source 1..19: mol_type = other DNA
gacaaggacgggctgaacc

SEQ ID NO: 139 - DNA, 20 bp, synthetic construct
  source 1..20: mol_type = other DNA
ccagggccgggtaaccactg

SEQ ID NO: 140 - DNA, 20 bp, synthetic construct
  source 1..20: mol_type = other DNA
gacattgtctatgcgtccgt

SEQ ID NO: 141 - DNA, 20 bp, synthetic construct
  source 1..20: mol_type = other DNA
ctcacctttggaaaacacag

SEQ ID NO: 142 - DNA, 20 bp, synthetic construct
  source 1..20: mol_type = other DNA
tgtccactgcacagttcgag

SEQ ID NO: 143 - DNA, 20 bp, synthetic construct
  source 1..20: mol_type = other DNA
ggcttgagagtcgaagtcga

SEQ ID NO: 144 - DNA, 20 bp, synthetic construct
  source 1..20: mol_type = other DNA
agaatgagtatgtcaagatg

SEQ ID NO: 145 - DNA, 20 bp, synthetic construct
  source 1..20: mol_type = other DNA
ccaccgaggcctgcgcaagg

SEQ ID NO: 146 - DNA, 20 bp, synthetic construct
  source 1..20: mol_type = other DNA
ccgtgtcattgcccacaccc

SEQ ID NO: 147 - DNA, 20 bp, synthetic construct
  source 1..20: mol_type = other DNA
gcgcaagcgacggccacatg

SEQ ID NO: 148 - DNA, 20 bp, synthetic construct
  source 1..20: mol_type = other DNA
ggctcggcctacattgagca

SEQ ID NO: 149 - RNA, 20 nt, synthetic construct
  source 1..20: mol_type = other RNA
acaaacgaccttgagcaggg

SEQ ID NO: 150 - DNA, 22 bp, synthetic construct
  source 1..22: mol_type = other DNA
accacaaggccctcagtacctc

SEQ ID NO: 151 - DNA, 22 bp, synthetic construct
  source 1..22: mol_type = other DNA
tgacagccttggtgttggagga

SEQ ID NO: 152 - DNA, 23 bp, synthetic construct
  source 1..23: mol_type = other DNA
gccgattgtgaacatggactacg

SEQ ID NO: 153 - DNA, 22 bp, synthetic construct
  source 1..22: mol_type = other DNA
gctcgtagttgagcacgctgaa

SEQ ID NO: 154 - DNA, 19 bp, synthetic construct
  source 1..19: mol_type = other DNA
gctccactgccttctgagt

SEQ ID NO: 155 - DNA, 21 bp, synthetic construct
  source 1..21: mol_type = other DNA
aataaaagcgatggcgattgg

SEQ ID NO: 156 - DNA, 20 bp, synthetic construct
  source 1..20: mol_type = other DNA
gcctcactccttttgcagac

SEQ ID NO: 157 - DNA, 20 bp, synthetic construct
  source 1..20: mol_type = other DNA
aagtggtcgttgagggcaat

SEQ ID NO: 158 - DNA, 20 bp, synthetic construct
  source 1..20: mol_type = other DNA
gtcacacccttctcccttcg

SEQ ID NO: 159 - DNA, 20 bp, synthetic construct
  source 1..20: mol_type = other DNA
caggtacaagctggaggtgg

SEQ ID NO: 160 - DNA, 20 bp, synthetic construct
  source 1..20: mol_type = other DNA
gcctcactccttttgcagac

SEQ ID NO: 161 - DNA, 20 bp, synthetic construct
  source 1..20: mol_type = other DNA
aagtggtcgttgagggcaat

SEQ ID NO: 162 - RNA, 11 nt, synthetic construct
  source 1..11: mol_type = other RNA
cctcctscctc

SEQ ID NO: 163 - DNA, 134 bp, synthetic construct
  source 1..134: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagct

SEQ ID NO: 164 - RNA, 11 nt, synthetic construct
  source 1..11: mol_type = other RNA
  misc_difference 1..11: note = This sequence may encompass 2-11 nucleotides
ttttttttttt

SEQ ID NO: 165 - RNA, 10 nt, synthetic construct
  source 1..10: mol_type = other RNA
tttttttttt

SEQ ID NO: 166 - RNA, 11 nt, synthetic construct
  source 1..11: mol_type = other RNA
ttttttttttt

SEQ ID NO: 167 - RNA, 12 nt, synthetic construct
  source 1..12: mol_type = other RNA
tttttttttttt

SEQ ID NO: 168 - RNA, 13 nt, synthetic construct
  source 1..13: mol_type = other RNA
ttttttttttttt

SEQ ID NO: 169 - DNA, 60 bp, synthetic construct
  source 1..60: mol_type = other DNA
tcccctgtccacctgctccagccactatccctctcccacttacagcagaagaaagggctg

SEQ ID NO: 170 - DNA, 61 bp, synthetic construct
  source 1..61: mol_type = other DNA
gtgacagaaggggaaagggtcctctgatcattgctcaccccacagagatctttgaaaggag

SEQ ID NO: 171 - DNA, 60 bp, synthetic construct
  source 1..60: mol_type = other DNA
tcaaccacagtggcatctgtgagctgcactgcccagccctggtcacctacaacacagaca

SEQ ID NO: 172 - DNA, 56 bp, synthetic construct
  source 1..56: mol_type = other DNA
tgcccccctgacctgcagcccccagcctggtatggagtccagtctaagcagactga

SEQ ID NO: 173 - DNA, 60 bp, synthetic construct
  source 1..60: mol_type = other DNA
ttactgggaccaggacccaccagagcggggggctccacccagcaccttcaaagggacacc

SEQ ID NO: 174 - DNA, 60 bp, synthetic construct
  source 1..60: mol_type = other DNA
accccgcgccccgcgccctcccagccgcgtccagccggagccatggggccggagccgcag

SEQ ID NO: 175 - DNA, 60 bp, synthetic construct
  source 1..60: mol_type = other DNA
aatctttgcgtgtgctggggggagcggcataattgtgctggtgcgtactcacgtggcaac

SEQ ID NO: 176 - DNA, 60 bp, synthetic construct
  source 1..60: mol_type = other DNA
catgaccaaaatcccttaacgtgagttttcgttccactgagcgtcagaccccgtagaaaa

SEQ ID NO: 177 - DNA, 130 bp, synthetic construct
  source 1..130: mol_type = other DNA
ttttttttaaacaaacattgtgttgacattaagaatgttggtttactttcaaatcggtcccctgtcgagttcggctctgggtgggcagtaggaccaccagtgtggggttctgctgggaccttggagagcc

SEQ ID NO: 178 - DNA, 128 bp, synthetic construct
  source 1..128: mol_type = other DNA
tgcatcccaggatgctgggtggccctgcagcctcctccacctcacctccatgacagcgctaaacgttggtgacggttgggagcctctggggctgttgaagtcaccttgtgtgttccaagtttccaaac

SEQ ID NO: 179 - DNA, 132 bp, synthetic construct
  source 1..132: mol_type = other DNA
aacagaaagtcattccttctttttaaaatggtgcttaagttccagcagatgccacataaggggtttgccatttgatacccctggggaacatttctgtaaataccattgacacatccgccttttgtatacatc

SEQ ID NO: 180 - DNA, 132 bp, synthetic construct
  source 1..132: mol_type = other DNA
ctgggtaatgagaggtggcttttgcggccagtattagactggaagttcatacctaagtactgtaataatacctcaatgtttgaggagcatgttttgtatacaaatatattgttaatctctgttatgtactgt

SEQ ID NO: 181 - DNA, 135 bp, synthetic construct
  source 1..135: mol_type = other DNA
actaattcttacactgcctgtatactttagtatgacgctgatacataactaaatttgatacttatattttcgtatgaaaatgagttgtgaaagttttgagtagatattactttatcactttttgaactaagaaac

SEQ ID NO: 182 - DNA, 36 bp, synthetic construct
  source 1..36: mol_type = other DNA
ttttgtaaagaaatttactatatatatatgcctttt

SEQ ID NO: 183 - DNA, 855 bp, synthetic construct
  source 1..855: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctgagcaatcacctatgaacttgctgcctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaactaagaaacttttgtaaagaaatttactatatatatatgcctctgc

SEQ ID NO: 184 - DNA, 159 bp, synthetic construct
  source 1..159: mol_type = other DNA
nnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannn

SEQ ID NO: 185 - DNA, 158 bp, synthetic construct
  source 1..158: mol_type = other DNA
nnnnnnnannnnnntctcnnncttcagcctcnatacgcgtgtcccggcnggacggtacttcctccnttctggtcctgcccccaggcccgtatagaagcttaaatgcaggagatgaacaatcatgggggaattttgcctcgatggcattgcgttgcana

SEQ ID NO: 186 - DNA, 74 bp, synthetic construct
  source 1..74: mol_type = other DNA
gggggcggagtcgcggacaggaaagtgccatcgaggcaaattcccccatctgcgtcagtcccctcaacttccgc

SEQ ID NO: 187 - protein, 4 aa, synthetic construct
LAEM

SEQ ID NO: 188 - RNA, 856 nt, synthetic construct
  source 1..856: mol_type = other RNA
nnnnnnnnnnnnnntcnctcancttcngcctctacacgggcctcgcggcagggcggtacttcagccattctggtcctgcccgcagatgcgtatnnnngcttgaatgcaggagctgaacaatcacctatgaattttgcctcgatggcattgcgttgannncctcctgcctccaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttcctcttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggagttcatacctaagtactgtaataatacctcaatgtttgaggagcatgcctcgtatacaaatatattgttaatctctgttatgtactgtactaattcttacactgcctgtatactttagtatgacgctgatacataactaaatttgatacttatattttcgtatgaaaatgagttgtgaaagttttgagtagatattacctcatcacctcttgaactaagaaacttttgtaaagaaatttactatatatatatgcctctgc

(19) United States
(12) Patent Application Publication
(10) Pub. No.: US 2026/0256949 A1
(43) Pub. Date: Sep. 3, 2026

(54) Compositions, Methods, And Systems For Targeting And Destabilizing RNA Transcripts (MYCN)

(71) Applicant: UTR Therapeutics Inc., New York, NY (US)

(72) Inventor: Chidiebere Awah, New York City, NY (US)

(21) Appl. No.: 19/534,082

(22) Filed: Feb. 9, 2026

Related U.S. Application Data
(60) Provisional application No. 63/757,767, filed on Feb. 12, 2025.

(51) Int. Cl. A61K 48/00; A61K 38/17; A61P 35/04

(52) U.S. Cl. CPC A61K 48/005; A61K 38/1709; A61P 35/04

(57) ABSTRACT

The present disclosure encompasses molecules, including DNA and RNA molecules, for destabilizing mRNA transcripts, along with the preparation and use thereof. Specifically encompassed are methods for preparing these molecules, methods for utilizing these molecules in therapies, particularly cancer therapies, and vectors, host cells, and kits for these preparations and uses.

### SEQUENCE LISTING

The instant application contains a Sequence Listing which has been submitted electronically in XML file format and is hereby incorporated by reference in its entirety. Said XML copy, created on Nov. 18, 2025, is named UTR1-002-00PUS_SL.xml and is 215,210 bytes in size.

### FIELD

The present disclosure relates to molecules for destabilizing specific mRNA transcripts, and their preparation and use. More specifically, the present disclosure relates to engineered 3′ untranslated region (3′UTR) molecules, compositions comprising these, as well as means for preparing and using these molecules and compositions.

### BACKGROUND

The ribosome is a conserved macromolecular machine that controls the translation and decoding of the mRNA encoded messages to proteins. This molecular function is not limited to making proteins but involves a delicate regulation and control of transcript quality assurances (1-3).

Infidel and aberrant mRNA transcripts are deleterious to the cells and hence ribosome quality assurance control dictates that fidel mRNA transcript are translated to proteins and in circumstances that the target mRNA for decoding and translation is not optimal, the ribosomes trigger many cellular machineries and mechanisms to degrade such mRNA.

Therefore, the cellular decision to translate or not translate, to degrade a transcript or not lies solely with the ribosome quality assurance machinery. However, despite this important function, it is not known how and what controls or dictates the ribosome fate switches from one cellular fate or state to another. The failure to understand this process remains an ongoing concern that has yet to be addressed.

There is also an ongoing need for molecules, compositions, and methods for utilisation in transcript targeting other procedures in RNA molecular biology. The present disclosure seeks to address these needs or at least to provide the public with a useful alternative.

### SUMMARY

In various aspects, the present disclosure encompasses:

A method of switching ribosomal function in a cell from (a) translation of a target mRNA to (b) degradation of the target mRNA, the method comprising: obtaining expression of one or more of: recombinant PELO protein, recombinant EXOSC4 protein, or recombinant RPL3 protein in the cell; and obtaining expression of a destabilizing mRNA in the cell, wherein the destabilizing mRNA specifically targets and destabilizes the target mRNA in the cell.

A method of switching of ribosomal function in a cancer cell from (a) translation of a target mRNA to (b) degradation of the target mRNA, the method comprising: obtaining expression of one or more of: recombinant PELO protein, recombinant EXOSC4 protein, or recombinant RPL3 protein in the cancer cell; and obtaining expression of a destabilizing mRNA in the cancer cell, wherein the destabilizing mRNA specifically targets and destabilizes the target mRNA in the cancer cell.

An expression system for switching ribosomal function in a cell from (a) translation of a target mRNA to (b) degradation of the target mRNA, the system comprising: one or more nucleic acid constructs for obtaining expression of recombinant PELO protein, recombinant EXOSC4 protein, or recombinant RPL3 protein in the cell, and one or more nucleic acid constructs for obtaining expression of a destabilizing mRNA which specifically targets and destabilizes the target mRNA in the cell.

A method for obtaining degradation of a target mRNA in a cell, the method comprising: having expression of one or more of: PELO protein, EXOSC4 protein, or RPL3 protein in the cell, wherein the PELO protein, EXOSC4 protein, and/or the RPL3 protein promote ribosomal mediated degradation of the target mRNA, and obtaining expression of a destabilizing mRNA in the cell, wherein the destabilizing mRNA specifically targets and destabilizes the target mRNA in the cell, wherein the destabilizing mRNA is a c-MYC destabilizing mRNA, wherein the target mRNA is a c-MYC target mRNA, and wherein: (i) the c-MYC destabilizing mRNA targets SEQ ID NO: 91 on exon 2 of the c-MYC target RNA; (ii) the c-MYC destabilizing mRNA targets SEQ ID NO: 92 on exons 1-3 and introns 1-2 of the c-MYC target RNA; or (iii) the c-MYC destabilizing mRNA targets a sequence comprising CACGUG and/or AACGUG on the c-MYC target RNA.

A method for obtaining degradation of a target mRNA in a cell, the method comprising: having expression of one or more of: PELO protein, EXOSC4 protein, or RPL3 protein in the cell, wherein the PELO protein, EXOSC4 protein, and/or the RPL3 protein promote ribosomal mediated degradation of the target mRNA, and obtaining expression of a destabilizing mRNA in the cell, wherein the destabilizing mRNA specifically targets and destabilizes the target mRNA in the cell, wherein the destabilizing mRNA is an ERBB2 destabilizing mRNA, the target mRNA is an ERBB2 target mRNA, and wherein: (i) the ERBB2 destabilizing mRNA targets SEQ ID NO: 93 on exons 2-7 and introns 2-6 of the ERBB2 target RNA; (ii) the ERBB2 destabilizing mRNA targets SEQ ID NO: 94 on exons 26-27 and intron 26 of the ERBB2 target RNA; or (iii) the ERBB2 destabilizing mRNA targets a sequence comprising CAGA on the ERBB2 target RNA.

In various aspects: The target mRNA is a c-MYC target mRNA and the destabilizing mRNA is a c-MYC destabilizing mRNA.

The target mRNA is a MYCN target mRNA and the destabilizing mRNA is a MYCN destabilizing mRNA.

The target mRNA is a ERBB2 target mRNA and the destabilizing mRNA is a ERBB2 destabilizing mRNA.

The expression of the recombinant PELO protein is obtained through one or more plasmid vectors that allow expression of the recombinant PELO protein.

The expression of the recombinant EXOSC4 protein is obtained through one or more plasmid vectors that allow expression of the recombinant EXOSC4 protein.

The expression of the recombinant RPL3 protein is obtained through one or more plasmid vectors that allow expression of the recombinant RPL3 protein.

The expression of the recombinant PELO protein, the recombinant EXOSC4 protein, and the recombinant RPL3 is obtained through one or more plasmid vectors that allow expression of the recombinant PELO protein, the recombinant EXOSC4 protein, and the recombinant RPL3 protein.

The expression of the destabilizing mRNA is obtained from one or more plasmid vectors that also allow expression of the recombinant PELO protein.

The expression of the destabilizing mRNA is obtained from one or more plasmid vectors that also allow expression of the recombinant EXOSC4 protein.

The expression of the destabilizing mRNA is obtained from one or more plasmid vectors that also allow expression of the recombinant RPL3 protein.

The expression of the destabilizing mRNA is obtained from one or more plasmid vectors that also allow expression of the recombinant PELO protein, the recombinant EXOSC4 protein, and the recombinant RPL3 protein.

The expression of the destabilizing mRNA is obtained from one or more plasmid vectors that are separate from the one or more plasmid vectors that allow expression of the recombinant PELO protein, the recombinant EXOSC4 protein, and/or the recombinant RPL3 protein.

The expression of the recombinant PELO protein is obtained through one or more integrated vector constructs in the cell that allow expression of the recombinant PELO protein.

The expression of the recombinant EXOSC4 protein is obtained through one or more integrated vector constructs in the cell that allow expression of the recombinant EXOSC4 protein.

The expression of the recombinant RPL3 protein is obtained through one or more integrated vector constructs in the cell that allow expression of the recombinant RPL3 protein.

The expression of the recombinant PELO protein, the recombinant EXOSC4 protein, and the recombinant RPL3 protein is obtained through one or more integrated vector constructs in the cell that allow expression of the recombinant PELO protein, the recombinant EXOSC4 protein, and the recombinant RPL3 protein.

The expression of the destabilizing mRNA is obtained from one or more integrated vector constructs in the cell which also allow expression of the recombinant PELO protein.

The expression of the destabilizing mRNA is obtained from one or more integrated vector constructs in the cell which also allow expression of the recombinant EXOSC4 protein.

The expression of the destabilizing mRNA is obtained from one or more integrated vector constructs in the cell which also allow expression of the recombinant RPL3 protein.

The expression of the destabilizing mRNA is obtained from one or more integrated vector constructs in the cell which also allow expression of the recombinant PELO protein, the recombinant EXOSC4 protein, and the recombinant RPL3 protein.

The expression of the destabilizing mRNA is obtained from one or more integrated vector constructs in the cell which are separate from the one or more plasmid vectors in the cell which allow expression of the recombinant PELO protein, the recombinant EXOSC4 protein, and/or the recombinant RPL3 protein.

In further aspects, the present disclosure encompasses:

A method of treating a cancer in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUUU, CCUCUGC, UAAGUUAU, UAACUUAU, GUAAAUAG, or CCUCCUGCCUC (SEQ ID NO: 53), wherein the cancer is a nervous system cancer, prostate cancer, kidney cancer, breast cancer, lung cancer, bone cancer, muscle cancer, or eye cancer, and wherein the cancer is characterized by overexpression of MYCN.

A method of treating a cancerous tumor in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUUU, CCUCUGC, UAAGUUAU, UAACUUAU, GUAAAUAG, or CCUCCUGCCUC (SEQ ID NO: 53), wherein the tumor is a nervous system tumor, prostate tumor, kidney tumor, lung tumor, breast tumor, bone tumor, muscle tumor, or eye tumor, wherein the tumor is characterized by overexpression of MYCN, and wherein the tumor is metastatic.

A method of preventing metastasis of a cancerous tumor in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUUU, CCUCUGC, UAAGUUAU, UAACUUAU, GUAAAUAG, or CCUCCUGCCUC (SEQ ID NO: 53), wherein the tumor is a nervous system tumor, prostate tumor, kidney tumor, lung tumor, breast tumor, bone tumor, muscle tumor, or eye tumor, and wherein the tumor is characterized by overexpression of MYCN.

A method of treating a cancer in a subject having cancer progression after chemotherapy treatment, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUUU, CCUCUGC, UAAGUUAU, UAACUUAU, GUAAAUAG, or CCUCCUGCCUC (SEQ ID NO: 53), wherein the cancer is a nervous system cancer, prostate cancer, kidney cancer, lung cancer, breast cancer, bone cancer, muscle cancer, or eye cancer, and wherein the cancer is characterized by overexpression of MYCN.

A method of treating a cancer in a subject, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUIUU, CCUCUGC, UAAGUUAU, UAACUUAU, GUAAAUAG, or CCUCCUGCCUC (SEQ ID NO: 53), wherein the cancer is a nervous system cancer, prostate cancer, kidney cancer, lung cancer, or breast cancer, wherein the cancer is characterized by overexpression of MYCN, and wherein the cancer is resistant to chemotherapy.

A method of reducing MYCN expression in a cancerous tumor in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUUU, CCUCUGC, UAAGUUAU, UAACUUAU, GUAAAUAG, or CCUCCUGCCUC (SEQ ID NO: 53), wherein the tumor is a nervous system tumor, prostate tumor, kidney tumor, lung tumor, breast tumor, bone tumor, muscle tumor, or eye tumor.

A DNA molecule comprising in the 5′ to 3′ direction of transcription relative to synthesis of a mRNA molecule comprising a 3′ UTR of an mRNA encoding a MYCN protein: (a) a promoter, operatively linked to: (b) a nucleic acid sequence encoding a 3′ UTR of a MYCN gene in the mRNA molecule in which one, or more ARE poly(U) stabilizing motifs of the 3′ UTR of the mRNA are substituted with a nucleic acid sequence CCUC, CUGC, ACCUC, AUUIUU, CCUCUGC, UAAGUUAU, UAACUUAU, GUAAAUAG, or CCUCCUGCCUC (SEQ ID NO: 53).

An RNA molecule comprising a 3′ UTR of a MYCN mRNA molecule in which one, or more ARE poly(U) stabilizing motifs of the 3′ UTR of the mRNA are substituted with a nucleic acid sequence CCUC, CUGC, ACCUC, AUUUU, CCUCUGC, UAAGUUAU, UAACUUAU, GUAAAUAG, or CCUCCUGCCUC (SEQ ID NO: 53).

Use of a plasmid vector for preparing a medicament for: (i) treating cancer in a subject in need thereof, (ii) treating cancer in a subject having cancer progression after chemotherapy treatment; or (ii) treating chemotherapy resistant cancer in a subject in need thereof; the plasmid vector comprising a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUIUU, CCUCUGC, UAAGUUAU, UAACUUAU, GUAAAUAG, or CCUCCUGCCUC (SEQ ID NO: 53), wherein the cancer is nervous system cancer, prostate cancer, kidney cancer, breast cancer, lung cancer, bone cancer, muscle cancer, or eye cancer, and wherein the cancer is characterized by overexpression of MYCN.

Use of a plasmid vector for preparing a medicament for: (i) treating a cancerous tumor in a subject in need thereof, (ii) preventing metastasis of a cancerous tumor in a subject in need thereof, or (iii) reducing c-MYC expression in a ovarian tumor in a subject in need thereof; the plasmid vector comprising a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUIUU, CCUCUGC, UAAGUUAU, UAACUUAU, GUAAAUAG, or CCUCCUGCCUC (SEQ ID NO: 53), wherein the cancer is nervous system cancer, prostate cancer, kidney cancer, breast cancer, lung cancer, bone cancer, muscle cancer, or eye cancer, and wherein the tumor is characterized by overexpression of MYCN.

In various aspects, the present disclosure encompasses:

A method of treating cancer in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a c-MYC protein: (a) a promoter, operatively linked to: (b) a nucleic acid sequence encoding a 3′ UTR of a c-MYC gene in the mRNA molecule in which one or more ARE poly(U) stabilizing motifs of the 3′ UTR of the mRNA are substituted with a nucleic acid sequence selected from CCUC, CUGC, ACCUC, AUUUU, UAAGUUAUG, UAACUUAU, UGCUGCCCU, GUAAAUAG, CCUCUGC, UCCUGCCCUC (SEQ ID NO: 50), CCUCGUAACUU (SEQ ID NO: 52), CCUCCUGCAUUU (SEQ ID NO: 54), UAAGUUAUCCUCUAUU (SEQ ID NO: 57), CUGCCUCUGCUAACUUAU (SEQ ID NO: 58), or CCUCCUGCCUC (SEQ ID NO: 53), wherein the cancer is breast cancer, ovarian cancer, or pancreatic cancer, and wherein the cancer is characterized by overexpression of c-MYC.

A method of treating a cancerous tumor in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a c-MYC protein: (a) a promoter, operatively linked to: (b) a nucleic acid sequence encoding a 3′ UTR of a c-MYC gene in the mRNA molecule in which one or more ARE poly(U) stabilizing motifs of the 3′ UTR of the mRNA are substituted with a nucleic acid sequence selected from CCUC, CUGC, ACCUC, AUUUU, UAAGUUAUG, UAACUUAU, UGCUGCCCU, GUAAAUAG, CCUCUGC, UCCUGCCCUC (SEQ ID NO: 50), CCUCGUAACUU (SEQ ID NO: 52), CCUCCUGCAUUU (SEQ ID NO: 54), UAAGUUAUCCUCUAUU (SEQ ID NO: 57), CUGCCUCUGCUAACUUAU (SEQ ID NO: 58), or CCUCCUGCCUC (SEQ ID NO: 53), wherein the tumor is a breast cancer tumor, an ovarian cancer tumor, or a pancreatic cancer tumor, wherein the tumor is characterized by overexpression of c-MYC, and wherein the tumor is metastatic.

A method of preventing metastasis of a cancerous tumor in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a c-MYC protein: (a) a promoter, operatively linked to: (b) a nucleic acid sequence encoding a 3′ UTR of a c-MYC gene in the mRNA molecule in which one or more ARE poly(U) stabilizing motifs of the 3′ UTR of the mRNA are substituted with a nucleic acid sequence selected from CCUC, CUGC, ACCUC, AUUIUU, UAAGUUAUG, UAACUUAU, UGCUGCCCU, GUAAAUAG, CCUCUGC, UCCUGCCCUC (SEQ ID NO: 50), CCUCGUAACUU (SEQ ID NO: 52), CCUCCUGCAUUU (SEQ ID NO: 54), UAAGUUAUCCUCUAUU (SEQ ID NO: 57), CUGCCUCUGCUAACUUAU (SEQ ID NO: 58), or CCUCCUGCCUC (SEQ ID NO: 53), wherein the tumor is a breast cancer tumor, an ovarian cancer tumor, or a pancreatic cancer tumor, and wherein the tumor is characterized by overexpression of c-MYC.

A method of treating cancer in a subject having cancer progression after chemotherapy treatment, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a c-MYC protein: (a) a promoter, operatively linked to: (b) a nucleic acid sequence encoding a 3′ UTR of a c-MYC gene in the mRNA molecule in which one or more ARE poly(U) stabilizing motifs of the 3′ UTR of the mRNA are substituted with a nucleic acid sequence selected from CCUC, CUGC, ACCUC, AUUUU, UAAGUUAUG, UAACUUAU, UGCUGCCCU, GUAAAUAG, CCUCUGC, UCCUGCCCUC (SEQ ID NO: 50), CCUCGUAACUU (SEQ ID NO: 52), CCUCCUGCAUUU (SEQ ID NO: 54), UAAGUUAUCCUCUAUU (SEQ ID NO: 57), CUGCCUCUGCUAACUUAU (SEQ ID NO: 58), or CCUCCUGCCUC (SEQ ID NO: 53), wherein the cancer is a breast cancer, an ovarian cancer, or a pancreatic cancer, and wherein the cancer is characterized by overexpression of c-MYC.

A method of treating cancer in a subject, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a c-MYC protein: (a) a promoter, operatively linked to: (b) a nucleic acid sequence encoding a 3′ UTR of a c-MYC gene in the mRNA molecule in which one or more ARE poly(U) stabilizing motifs of the 3′ UTR of the mRNA are substituted with a nucleic acid sequence selected from CCUC, CUGC, ACCUC, AUUIUU, UAAGUUAUG, UAACUUAU, UGCUGCCCU, GUAAAUAG, CCUCUGC, UCCUGCCCUC (SEQ ID NO: 50), CCUCGUAACUU (SEQ ID NO: 52), CCUCCUGCAUUU (SEQ ID NO: 54), UAAGUUAUCCUCUAUU (SEQ ID NO: 57), CUGCCUCUGCUAACUUAU (SEQ ID NO: 58), or CCUCCUGCCUC (SEQ ID NO: 53), wherein the cancer is a breast cancer, an ovarian cancer, or a pancreatic cancer, wherein the cancer is characterized by overexpression of c-MYC, and wherein the cancer is resistant to chemotherapy.

A method of reducing c-MYC expression in a cancerous tumor in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a c-MYC protein: (a) a promoter, operatively linked to: (b) a nucleic acid sequence encoding a 3′ UTR of a c-MYC gene in the mRNA molecule in which one or more ARE poly(U) stabilizing motifs of the 3′ UTR of the mRNA are substituted with a nucleic acid sequence selected from CCUC, CUGC, ACCUC, AUUIUU, UAAGUUAUG, UAACUUAU, UGCUGCCCU, GUAAAUAG, CCUCUGC, UCCUGCCCUC (SEQ ID NO: 50), CCUCGUAACUU (SEQ ID NO: 52), CCUCCUGCAUUU (SEQ ID NO: 54), UAAGUUAUCCUCUAUU (SEQ ID NO: 57), CUGCCUCUGCUAACUUAU (SEQ ID NO: 58), or CCUCCUGCCUC (SEQ ID NO: 53), wherein the tumor is a breast cancer tumor, an ovarian tumor, or a pancreatic tumor.

Use of a plasmid vector for preparing a medicament for: (i) treating cancer in a subject in need thereof, (ii) treating cancer in a subject having cancer progression after chemotherapy treatment; or (ii) treating chemotherapy resistant cancer in a subject in need thereof; the plasmid vector comprising a DNA molecule, wherein the DNA molecule comprises in the 5′ to 3′ direction of transcription relative to synthesis of a messenger RNA (mRNA) molecule comprising a 3′ UTR of an mRNA encoding a c-MYC protein: (a) a promoter, operatively linked to: (b) a nucleic acid sequence encoding a 3′ UTR of a c-MYC gene in the mRNA molecule in which one or more ARE poly(U) stabilizing motifs of the 3′ UTR of the mRNA are substituted with a nucleic acid sequence selected from CCUC, CUGC, ACCUC, AUUIUU, UAAGUUAUG, UAACUUAU, UGCUGCCCU, GUAAAUAG, CCUCUGC, UCCUGCCCUC (SEQ ID NO: 50), CCUCGUAACUU (SEQ ID NO: 52), CCUCCUGCAUUU (SEQ ID NO: 54), UAAGUUAUCCUCUAUU (SEQ ID NO: 57), CUGCCUCUGCUAACUUAU (SEQ ID NO: 58), or CCUCCUGCCUC (SEQ ID NO: 53), wherein the cancer is a breast cancer, an ovarian cancer, or a pancreatic cancer, and wherein the cancer is characterized by overexpression of c-MYC.

Use of a plasmid vector for preparing a medicament for: (i) treating a cancerous tumor in a subject in need thereof, (ii) preventing metastasis of a cancerous tumor in a subject in need thereof, or (iii) reducing c-MYC expression in a cancerous tumor in a subject in need thereof; the plasmid vector comprising a DNA molecule, wherein the DNA molecule comprises in the 5′ to 3′ direction of transcription relative to synthesis of a messenger RNA (mRNA) molecule comprising a 3′ UTR of an mRNA encoding a c-MYC protein: (a) a promoter, operatively linked to: (b) a nucleic acid sequence encoding a 3′ UTR of a c-MYC gene in the mRNA molecule in which one or more ARE poly(U) stabilizing motifs of the 3′ UTR of the mRNA are substituted with a nucleic acid sequence selected from CCUC, CUGC, ACCUC, AUUIUU, UAAGUUAUG, UAACUUAU, UGCUGCCCU, GUAAAUAG, CCUCUGC, UCCUGCCCUC (SEQ ID NO: 50), CCUCGUAACUU (SEQ ID NO: 52), CCUCCUGCAUUU (SEQ ID NO: 54), UAAGUUAUCCUCUAUU (SEQ ID NO: 57), CUGCCUCUGCUAACUUAU (SEQ ID NO: 58), or CCUCCUGCCUC (SEQ ID NO: 53), wherein the tumor is a breast cancer tumor, an ovarian tumor, or a pancreatic tumor, and wherein the tumor is characterized by overexpression of c-MYC.

In various aspects, the present disclosure encompasses:

An RNA molecule comprising or consisting of any one of SEQ ID NO: 1-3.

An RNA molecule comprising or consisting of a nucleotide sequence having at least 80%, at least 85%, at least 90%, at least 95%, or at least 99% identity to any one of SEQ ID NO: 1-3.

A DNA molecule comprising or consisting of any one of SEQ ID NO: 4-27.

A DNA molecule comprising or consisting of a nucleotide sequence having at least 80%, at least 85%, at least 90%, at least 95%, or at least 99% identity to any one of SEQ ID NO: 4-27.

A DNA molecule comprising or consisting of any one of SEQ ID NO: 30-48.

A DNA molecule comprising or consisting of a nucleotide sequence having at least 80%, at least 85%, at least 90%, at least 95%, or at least 99% identity to any one of SEQ ID NO: 30-48.

A DNA molecule comprising or consisting of any one of SEQ ID NO: 30-48.

A DNA molecule comprising or consisting of a nucleotide sequence having at least 80%, at least 85%, at least 90%, at least 95%, or at least 99% identity to any one of SEQ ID NO: 30-48.

A protein molecule comprising or consisting of any one of SEQ ID NO: 72-90.

A protein molecule comprising or consisting of an amino acid sequence having at least 80%, at least 85%, at least 90%, at least 95%, or at least 99% identity to any one of SEQ ID NO: 72-90.

A recognition sequence comprising or consisting of any one of SEQ ID NO: 91-94.

A recognition sequence comprising or consisting of a nucleotide sequence having at least 80%, at least 85%, at least 90%, at least 95%, or at least 99% identity to any one of SEQ ID NO: 91-94.

A plasmid vector comprising the DNA molecule of any preceding aspect.

A host cell comprising the DNA molecule of a preceding aspect or the plasmid vector of a preceding aspect.

A pharmaceutical composition comprising the DNA molecule of a preceding aspect or the plasmid vector of a preceding aspect.

The pharmaceutical composition of a preceding aspect which is formulated for oral, intravenous, intraperitoneal, subcutaneous, intramuscular, intrathecal intracranial, intraspinal, intrarectal, transperineal, or transurethral administration.

The pharmaceutical composition of a preceding aspect which is formulated for administration at least once per week, at least twice per week, at least three times per week, or at least every other day.

The plasmid vector comprised in the pharmaceutical composition is administered to the subject at a dosage of about 2 μg to about 240 μg;

The plasmid vector comprised in the pharmaceutical composition is administered to the subject at a dosage of about 4 μg to about 320 μg;

The plasmid vector comprised in the pharmaceutical composition is administered to the subject at a dosage of about 10 μg to about 430 μg; or

The plasmid vector comprised in the pharmaceutical composition is administered to the subject at a dosage of about 5 μg to about 960 μg.

The pharmaceutical composition is administered to the subject over a treatment period of at least one week;

The pharmaceutical composition is administered to the subject over a treatment period of at least two weeks;

The pharmaceutical composition is administered to the subject over a treatment period of at least three weeks; or

The pharmaceutical composition is administered to the subject over a treatment period of at least four weeks.

The pharmaceutical composition is administered such that: (i) after the treatment period, there is a treatment break of at least one week; (ii) after the treatment period, there is a treatment break of at least two weeks; (iii) after the treatment period, there is a treatment break of at least three weeks; (iv) after the treatment period, there is a treatment break of at least four weeks.

The pharmaceutical composition is administered such that: (i) the pharmaceutical composition is administered to the subject in at least one treatment cycle; (ii) the pharmaceutical composition is administered to the subject in at least two treatment cycles; (iii) the pharmaceutical composition is administered to the subject in at least three treatment cycles; (iv) the pharmaceutical composition is administered to the subject in at least four treatment cycles;

The pharmaceutical composition of a preceding aspect which is (i) formulated for co-administration with a drug selected from the group consisting of PARP inhibitors, TOP2 inhibitors, taxanes, intercalating agents, alkylating agents, HER2/neu/topoisomerase antibody-drug conjugates, EGFR inhibitors, VEGF inhibitors, anti-androgen hormone therapies including AR inhibitors and MYC-Max protein inhibitor agents; (ii) formulated for co-administration with a drug selected from the group consisting of an anti-PD-1 monoclonal antibody, an anti-PD-L1 monoclonal antibody, and a checkpoint inhibitor; (iii) formulated for co-administration with a drug selected from the group consisting of olaparib, epirubicin, paclitaxel, cisplatin, doxorubicin, trastuzumab deruxtecan, osimertinib, bevacizumab, abiraterone, enzalutamide, and MYCi975; (iv) formulated for co-administration with a drug selected from the group consisting of fluorouracil, methotrexate, capecitabine, carboplatin, cyclophosphamide, oxaliplatin, altretamine, bendamustine, busulfan, chlorambucil, daunorubicin, gemcitabine, idarubicin, ifosfamide, mitoxantrone, cabazitaxel, ceritinib, cladribine, dacarbazine, and docetaxel; or (v) formulated for co-administration co-administered with a drug selected from a group consisting of pembrolizumab, nivolumab, brexucabtagene autoleucel, ado-trastuzumab emtansine, aldesleukin, amivantamab-vmjw, atezolizumab, axicabtagene ciloleucel, blinatumomab, cetuximab, daratumumab, durvalumab, elotuzumab, gemtuzumab ozogamicin, ipilimumab, mogamulizumab, naxitamab, obinutuzumab, ramucirumab, and siltuximab.

The kit of a preceding aspect, which further comprises: (i) one or more additional cancer drugs; (ii) one or more drugs selected from the group consisting of PARP inhibitors, TOP2 inhibitors, taxanes, intercalating agents, alkylating agents, HER2/neu/topoisomerase antibody-drug conjugates, EGFR inhibitors, VEGF inhibitors, anti-androgen hormone therapies including AR inhibitors and MYC-Max protein inhibitor agents; or (iii) one or more drugs selected from the group consisting of an anti-PD-1 monoclonal antibody, an anti-PD-L1 monoclonal antibody, and a checkpoint inhibitor.

A kit comprising one or more of: (i) the DNA molecule of a preceding aspect; (ii) the plasmid vector of a preceding aspect; (iii) the host cell of a preceding aspect; or (iv) the pharmaceutical composition of any preceding aspect; and, optionally, instructions for use.

In various aspects:

The cancer cell is from a cancer selected from the group consisting of breast cancer, colorectal cancer, lung cancer, nervous system cancer, ovarian cancer, pancreatic cancer, and prostate cancer.

The cancer cell is from a cancer selected from the group consisting of bone cancer, cervical cancer, endometrial cancer, gastrointestinal cancer, hematological cancer, liver cancer, penile cancer, skin cancer, thyroid cancer, and testicular cancer.

The cancer cell is from a cancer selected from the group consisting of breast adenocarcinoma, colon adenocarcinoma, lung squamous carcinoma, lung adenocarcinoma, medulloblastoma, neuroblastoma ovarian adenocarcinoma, pancreatic adenocarcinoma, pancreatic cystadenocarcinoma, pancreatic acinar cell carcinoma, prostate adenocarcinoma, and rhabdomyosarcoma.

The cancer cell is from a cancer selected from the group consisting of acute myeloid leukemia, bladder adenocarcinoma, diffuse B cell lymphoma, esophageal carcinoma, endometrial adenocarcinoma, gastric adenoma, gastric carcinoma, hepatocellular carcinoma, osteosarcoma, retinoblastoma, uterine carcinosarcoma, and Wilms tumor. The cancer is selected from the group consisting of nervous system cancer, prostate cancer, kidney cancer, breast cancer, lung cancer, bone cancer, muscle cancer, and eye cancer.

The cancer is selected from the group consisting of neuroblastoma, medulloblastoma, retinoblastoma, hemangioblastoma, oligodendroglioma, astrocytoma, ependymoma, and glioblastoma multiforme.

The cancer is characterized by tumors selected from the group consisting of glioma tumors, non-glioma tumors, and rhabdoid tumors.

The cancer is selected from the group consisting of neuroendocrine prostate cancer, prostate adenocarcinoma, and androgen independent prostate cancer.

The cancer is selected from Wilms tumor, small cell lung cancer, and triple negative breast cancer.

The cancer is selected from rhabdomyosarcoma and osteosarcoma.

The cancer is selected from the group consisting of breast cancer, ovarian cancer, and pancreatic cancer.

The ovarian cancer is selected from the group consisting of epithelial ovarian carcinoma and peritoneal carcinoma.

The ovarian cancer is a high grade serous ovarian carcinoma.

The ovarian cancer is characterized by tumors selected from the group consisting of ovarian adenocarcinoma tumors, germ cell tumors, and stromal cell tumors;

The pancreatic cancer is selected from the group consisting of pancreatic adenocarcinoma, pancreatic cystadenocarcinoma, and pancreatic acinar cell carcinoma.

The pancreatic cancer is characterized by tumors selected from the group consisting of islet cell tumors, non-endocrine pancreatic tumors, and neuroendocrine pancreatic tumors.

The cancer in the subject is resistant to a drug selected from the group consisting of paclitaxel, cisplatin, olaparib, doxorubicin, bevacizumab, trastuzumab, and trastuzumab deruxtecan;

The cancer in the subject is resistant to a drug selected from the group consisting of fluorouracil, methotrexate, capecitabine, carboplatin, cyclophosphamide, oxaliplatin, altretamine, bendamustine, busulfan, chlorambucil, daunorubicin, gemcitabine, idarubicin, ifosfamide, mitoxantrone, cabazitaxel, ceritinib, cladribine, dacarbazine, and docetaxel;

The cancer in the subject is resistant to a drug selected from the group consisting of an anti-PD-1 monoclonal antibody, an anti-PD-L1 monoclonal antibody, and a checkpoint inhibitor; or

The cancer in the subject is resistant to a drug selected from a group consisting of pembrolizumab, nivolumab, brexucabtagene autoleucel, ado-trastuzumab emtansine, aldesleukin, amivantamab-vmjw, atezolizumab, axicabtagene ciloleucel, blinatumomab, cetuximab, daratumumab, durvalumab, elotuzumab, gemtuzumab ozogamicin, ipilimumab, mogamulizumab, naxitamab, obinutuzumab, ramucirumab, and siltuximab.

While specific polynucleotide and polypeptide sequences have been noted in the preceding aspects, other sequences as disclosed herein may be substituted for or added to the sequences in the preceding aspects, and such will be understood as defining further aspects that are encompassed by the present disclosure. Novel features that are believed to be characteristic will be better understood from the detailed description when considered in connection with any accompanying figures and examples. However, the figures and examples provided herein are intended to help illustrate or assist with developing an understanding of this disclosure; these are not intended to limit the scope of this disclosure. The disclosure of U.S. Application No. 63/757,767 is incorporated by reference herein.

### BRIEF DESCRIPTION OF THE DRAWINGS

FIG. 1. Preparation of the ERBB2 and MYC plasmid system used in determining the target transcript specificity for the 3′UTR destabilized mRNA. (A) Illustration of the HEK293T cells transfected with the pYFP-ERBB2, pYFP-ERBB2 delta C990, pYFP-ERBB2 delta C776 respectively, the cells were expanded, and the flow cytometry sorting was used to select only YFP-positive cells. (B) Image shows WT HEK293T negative for YFP. (C) Image shows HEK293T cells transfected with the pYFP-ERBB2, YFP positive cells sorted. (D) The image shows HEK293T cells transfected with the pYFP-ERBB2 deltaC990, YFP positive cells sorted. (E) The image shows HEK293T cells transfected with the pYFP-ERBB2 deltaC776, YFP positive cells sorted. (F) The image shows the structure of the ERBB2 gene. (G) The image shows the pYFP-ERBB2 cDNA sequences maps to the first ERBB2 exon. (H) The image shows the pYFP-ERBB2 delta C990 cDNA sequences maps to the maps to the ERBB2 exon (2-7, introns 2-6). (I) The image shows the pYFP-ERBB2 delta C776 cDNA sequences maps to the ERBB2 exon. (26-27, intron 26). (J) Illustration of the HEK293T cells transfected with the pCL20-mEGFP-MYC-MYC and pZGreen1-cMYC/pLVX-puromycin respectively, the cells were expanded, and the flow cytometry sorting was used to select only GFP-positive cells. (K) Image shows WT HEK293T negative for GFP. (L) Image shows HEK293T cells transfected with the pCL20-mEGFP-MYC-MYC, GFP positive cells sorted. (M) Image shows HEK293T cells transfected with the pZGreen1-cMYC/pLVX-puromycin, GFP positive cells sorted. (N) The image shows the pZGreen1-cMYC cDNA sequences maps to the MYC exon 1-3 and introns 1-2. (O) The image shows the pCL20-mEGFP-MYC-MYC cDNA sequences maps to 132 bp sequences within c-MYC exon 2.

FIG. 2. 3′UTRMYC1-18 is specific in the recognition of the target c-MYC sites. (A) Illustration of the HEK293T cells transfected with the pZsGreen1-c-MYC/pLVX puromycin with no treatment. (B) Illustration of the HEK293T cells transfected with the pZsGreen1-c-MYC/pLVX puromycin treated with the empty vector. (C) Illustration of the HEK293T cells transfected with the pZsGreen1-c-MYC/pLVX puromycin treated with the 3′UTRMYC1-18 mRNA destabilizing drugs. (D) Flow cytometry image of the HEK293T positive for GFP due to pZsGreen1-c-MYC/pLVX puromycin with no treatment and no label with the c-MYC-647. (E) Flow cytometry image of the HEK293T positive for GFP due to pZsGreen1-c-MYC/pLVX puromycin treated with the empty vector and no label with c-MYC-647. (F) Flow cytometry image of the HEK293T positive for GFP due to pZsGreen1-c-MYC/pLVX puromycin treated with the 3′UTRMYC1-18 c-MYC mRNA destabilizing drug and no label with c-MYC-647. (G) The bar chart shows the quantification of the z-green, fluorescent intensity in the no treatment (red), empty vector treated (blue) and the 3′UTRMYC1-18 treated (green) HEK293T cells (N=2). (H) Flow cytometry image of the HEK293T transfected with the pZsGreen1-c-MYC/pLVX puromycin with no treatment but labeled with c-MYC-647 and with remarkably high c-MYC expression. (I) Flow cytometry image of the HEK293T transfected with the pZsGreen1-c-MYC/pLVX puromycin treated with the empty vector and labeled with c-MYC-647 and with extremely high c-MYC expression. (J) Flow cytometry image of the HEK293T transfected with the pZsGreen1-c-MYC/pLVX puromycin treated with the 3′UTRMYC1-18 mRNA destabilizing drug and labeled with c-MYC-647 and shows very reduced c-MYC expression. (K) The bar chart shows the quantification of the c-MYC expression in fluorescent intensity in the no treatment (red), empty vector treated (blue) and the 3′UTRMYC1-18 treated (green) HEK293T cells and labeled with the c-MYC-647 (N=2). (L) Illustration of the HEK293T cells transfected with the pCL20-mEGFP-MYC-MYC with no treatment. (M) Illustration of the HEK293T cells transfected with the pCL20-mEGFP-MYC-MYC and treated with the empty vector. (N) Illustration of the HEK293T cells transfected with the pCL20-mEGFP-MYC-MYC and treated with the 3′UTRMYC1-18 mRNA destabilizing drug. (O) Flow cytometry image of the HEK293T positive for GFP due to pCL20-mEGFP-MYC-MYC with no treatment and no label with the c-MYC-647. (P) Flow cytometry image of the HEK293T positive for GFP due to pCL20-mEGFP-MYC-MYC treated with the empty vector and no label with c-MYC-647. (Q) Flow cytometry image of the HEK293T positive for GFP due to pCL20-mEGFP-MYC-MYC treated with the 3′UTRMYC1-18 c-MYC mRNA destabilizing drug and no label with c-MYC-647. (R) The bar chart shows the quantification of the mEGFP expression in fluorescent intensity in the no treatment (red), empty vector treated (blue) and the 3′UTRMYC1-18 treated (green) HEK293 T cells (N=2). (S) Flow cytometry image of the HEK293 T transfected with the pCL20-mEGFP-MYC-MYC with no treatment but labeled with c-MYC-647 and with remarkably high c-MYC expression. (T) Flow cytometry image of the HEK293T transfected with the pCL20-mEGFP-MYC-MYC treated with the empty vector and labeled with c-MYC-647 and with high c-MYC expression. (U) Flow cytometry image of the HEK293T transfected with the pCL20-mEGFP-MYC-MYC treated with the 3′UTRMYC1-18 mRNA destabilizing drug and labeled with c-MYC-647 and shows complete loss of c-MYC expression. (V) The bar chart shows the quantification of the c-MYC expression in fluorescent intensity in the no treatment (red), empty vector treated (blue) and the 3′UTRMYC1-18 treated (green) HEK293T cells and labeled with the c-MYC-647 (N=2). *p=0.013, **p=0.0034 Two tailed T-test.

FIG. 3. The desARE3′UTRERBB2-30 specificity in the recognition of the target ERBB2 sites. (A) Flow cytometry image of the HEK293T positive for YFP due to pYFP-ERBB2 with no treatment and no label with the ERBB2-647. (B) Flow cytometry image of the HEK293T positive for YFP due to pYFP-ERBB2 treated with the empty vector and no label with ERBB2-647. (C) Flow cytometry image of the HEK293T positive for YFP due to pYFP-ERBB2 treated with the desARE3′UTRERBB2-30 ERBB2 mRNA destabilizing drug and no label with ERBB2-647. (D) The bar chart shows the quantification of the YFP fluorescent intensity in the no treatment (red), empty vector treated (blue) and the desARE3′UTRERBB2-30 treated (green) HEK293T cells (N=2). (E) Flow cytometry image of the HEK293T transfected with the pYFP-ERBB2 with no treatment but labeled with ERBB2-647 and with extremely high ERBB2 expression. (F) Flow cytometry image of the HEK293T transfected with the pYFP-ERBB2 treated with the empty vector and labeled with ERBB2-647 and with remarkably high ERBB2 expression. (G) Flow cytometry image of the HEK293T transfected with the pYFP-ERBB2 treated with the desARE3′UTRERBB2-30 mRNA destabilizing drug and labeled with ERBB2-647 and shows remarkably high ERBB2 expression. (H) The bar chart shows the quantification of the ERBB2 expression in fluorescent intensity in the no treatment (red), empty vector treated (blue) and the desARE3′UTRERBB2-30 treated (green) HEK293T cells and labeled with the ERBB2-647 (N=2). (I) Flow cytometry image of the HEK293T positive for YFP due to pYFP-ERBB2 delta C990 with no treatment and no label with the ERBB2-647. (J) Flow cytometry image of the HEK293T positive for YFP due to pYFP-ERBB2 delta C990 treated with the empty vector and no label with ERBB2-647. (K) Flow cytometry image of the HEK293T positive for YFP due to pYFP-ERBB2 delta C990 treated with the desARE3′UTRERBB2-30 ERBB2 mRNA destabilizing drug and no label with ERBB2-647. (L) The bar chart shows the quantification of the YFP fluorescent intensity in the no treatment (red), empty vector treated (blue) and the desARE3′UTRERBB2-30 treated (green) HEK293T cells (N=2). (M) Flow cytometry image of the HEK293T transfected with the pYFP-ERBB2 delta C990 with no treatment but labeled with ERBB2-647 and with remarkably high ERBB2 expression. (N) Flow cytometry image of the HEK293T transfected with the pYFP-ERBB2 delta C990 treated with the empty vector and labeled with ERBB2-647 and with remarkably high ERBB2 expression. (O) Flow cytometry image of the HEK293T transfected with the pYFP-ERBB2 delta C990 treated with the desARE3′UTRERBB2-30 mRNA destabilizing drug and labeled with ERBB2-647 and shows complete loss of the ERBB2 expression. (P) The bar chart shows the quantification of the ERBB2 expression in fluorescent intensity in the no treatment (red), empty vector treated (blue) and the desARE3′UTRERBB2-30 treated (green) HEK293T cells and labeled with the ERBB2-647 (N=2). (Q) Flow cytometry image of the HEK293T positive for YFP due to pYFP-ERBB2 delta C776 with no treatment and no label with the ERBB2-647. (R) Flow cytometry image of the HEK293T positive for YFP due to pYFP-ERBB2 delta C776 treated with the empty vector and no label with ERBB2-647. (S) Flow cytometry image of the HEK293T positive for YFP due to pYFP-ERBB2 delta C776 treated with the desARE3′UTRERBB2-30 ERBB2 mRNA destabilizing drug and no label with ERBB2-647. (T) The bar chart shows the quantification of the YFP fluorescent intensity in the no treatment (red), empty vector treated (blue) and the desARE3′UTRERBB2-30 treated (green) HEK293T cells (N=2). (U) Flow cytometry image of the HEK293T transfected with the pYFP-ERBB2 delta C776 with no treatment but labeled with ERBB2-647 and with remarkably high ERBB2 expression. (V) Flow cytometry image of the HEK293T transfected with the pYFP-ERBB2 delta C776 treated with the empty vector and labeled with ERBB2-647 and with moderate ERBB2 expression. (W) Flow cytometry image of the HEK293T transfected with the pYFP-ERBB2 delta C776 treated with the desARE3′UTRERBB2-30 mRNA destabilizing drug and labeled with ERBB2-647 and shows complete loss of the ERBB2 expression. (X) The bar chart shows the quantification of the ERBB2 expression in fluorescent intensity in the no treatment (red), empty vector treated (blue) and the desARE3′UTRERBB2-30 treated (green) HEK293T cells and labeled with the ERBB2-647 (N=2). **p=0.0025, ***p=0.0004, ****p<0.00001, p=ns=non-significant, Two tailed T-test.

FIG. 4. CRISPR KO of EXOSC4 and PELO validates evidence of EXOSC4, PELO as molecular regulators of ribosome fate switch by the engineered destabilized 3′UTR. (A) Bar chart shows the mRNA expression of the ERBB2 in WT NSCLC NCI H1975, desARE3′UTRERBB2-30, non-targeting control cells and in PELO, EXOSC4, MRT04, RPL3, HBSIL edited cells. (B) Bar chart shows the mRNA expression of the EXOSC4 in WT NSCLC NCI H1975, desARE3′UTRERBB2-30, non-targeting control cells and in PELO, EXOSC4, MRT04, RPL3, HBSIL edited cells. (C) Bar chart shows the mRNA expression of the PELO in desARE3′UTRERBB2-30, non-targeting control cells and in PELO, EXOSC4, MRT04, RPL3, HBSIL edited cells. (D) Bar chart shows the cell viability in WT NSCLC NCI H1975, desARE3′UTRERBB2-30, non-targeting control cells and in PELO, EXOSC4, MRT04, RPL3, HBSIL edited cells. ****p=<0.000134, Two tailed T-test.

FIG. 5. Overexpression of EXOSC4 and PELO in a CRISPR KO EXOSC4, PELO desARE3′UTRERBB2-30 cells restore the degrading and destabilizing function of the engineered desARE 3′UTRERBB2-30 constructs. (A) Bar charts show the ERBB2 mRNA expression in the WT, desARE3′UTRERBB2-30, non-targeting control, sgRNA (EXOSC4+PELO), sgRNA (EXOSC4+PELO)+(OE EXOSC4+PELO), sgRNA (EXOSC4+PELO)+(OE LSM10+RPL3), and in the sgRNA (EXOSC4+PELO)+OE (EXOSC4+PELO+LSM10+RPL3). (B) Western blot image of the ERBB2 and the GAPDH protein expression level in the Vector, non-targeting control, desARE3′UTRERBB2-30, sgRNA (EXOSC4+PELO), sgRNA (EXOSC4+PELO)+(OE EXOSC4+PELO) in the desARE3′UTRERBB2-30 cells. (C) Bar charts show the viability of the WT, vector, desARE3′UTRERBB2-30, non-targeting control, sgRNA (EXOSC4+PELO), sgRNA (EXOSC4+PELO)+(OE EXOSC4+PELO), sgRNA (EXOSC4+PELO)+(OE LSM10+RPL3), and in the sgRNA (EXOSC4+PELO)+OE (EXOSC4+PELO+LSM10+RPL3). (D) The bar chart of the ERBB2 protein expression normalized against the GAPDH protein expression level in the Vector, non-targeting control, desARE3′UTRERBB2-30, sgRNA (EXOSC4+PELO), sgRNA (EXOSC4+PELO)+(OE EXOSC4+PELO) in the desARE3′UTRERBB2-30 cells. ***p=0.0001, ****p<0.000024, Two tailed T-test.

FIG. 6. Validation of EXOSC4 and PELO over expression and stoichiometry in the transfected cells. (A) Bar charts show the EXOSC4 mRNA expression in the WT, desARE3′UTRERBB2-30, non-targeting control, sgRNA (EXOSC4+PELO), sgRNA (EXOSC4+PELO)+(OE EXOSC4+PELO), sgRNA (EXOSC4+PELO)+(OE LSM10+RPL3), and in the sgRNA (EXOSC4+PELO)+OE (EXOSC4+PELO+LSM10+RPL3). (B) Bar charts show the PELO mRNA expression in the WT, desARE3′UTRERBB2-30, non-targeting control, sgRNA (EXOSC4+PELO), sgRNA (EXOSC4+PELO)+(OE EXOSC4+PELO), sgRNA (EXOSC4+PELO)+(OE LSM10+RPL3), and in the sgRNA (EXOSC4+PELO)+OE (EXOSC4+PELO+LSM10+RPL3). ***p=0.00022, Two tailed T-test.

FIG. 7. Expression levels of RPL3 and LSM10. (A) Bar charts show the RPL3 mRNA expression in the WT, desARE3′UTRERBB2-30, non-targeting control, sgRNA (EXOSC4+PELO), sgRNA (EXOSC4+PELO)+(OE EXOSC4+PELO), sgRNA (EXOSC4+PELO)+(OE LSM10+RPL3), and in the sgRNA (EXOSC4+PELO)+OE (EXOSC4+PELO+LSM10+RPL3). (B) Bar charts show the LSM10 mRNA expression in the WT, desARE3′UTRERBB2-30, non-targeting control, sgRNA (EXOSC4+PELO), sgRNA (EXOSC4+PELO)+(OE EXOSC4+PELO), sgRNA (EXOSC4+PELO)+(OE LSM10+RPL3), and in the sgRNA (EXOSC4+PELO)+OE (EXOSC4+PELO+LSM10+RPL3). **p=0.003, ***p=0.0001, Two tailed T-test.

FIG. 8. The gene ontology of the destabilized mRNA destabilizing drugs and the response elements that are recognized. (A) Bar chart shows the rank order of the gene ontology theme down regulated in the cancer cells treated with the desARE3′UTRERBB2-30; the inverted arrow indicates that sequence specific regulatory elements are being recognized by the mRNA destabilizing drugs. (B) Image shows the rank order of the gene ontology theme down regulated in the 3′UTRMYC1-18 treated cells with the arrows indicating specific regulatory elements recognized by the mRNA destabilizing drug. (C) The bar chart shows the rank order of gene ontology theme upregulated in the desARE3′UTRERBB2-30 treated cancer cells. The purple inverted arrow indicates pathways experimentally validated and implicated in the mRNA destabilizing drug mechanism of action. (D) Image of sequence alignment shows the ERBB2 response element CAGA found on the Exons 2-6, and introns 2-5 which the ERBB2 mRNA destabilizing drug recognized. (E) Image of sequence alignment shows the ERBB2 response element CAGA on the exon 26-27 and introns 26 recognized by the ERBB2 mRNA destabilizing drug. (F) Image of sequence alignment shows that the ERBB2 response element CAGA is not in 5′ exon 1 and thus not recognized by the ERBB2 mRNA destabilizing drug. (G) The image of sequence alignment shows the MYC E-box canonical CACGTG and the non-canonical AACGTG sequences.

FIG. 9. Identification of molecular mediators of mRNA transcript degradation through 3′UTR destabilization. (A) Schematics depict the approach used in identifying the genetic pathways of 3′UTR end stalling, exon mRNA stalling, ribosome in frame stalling and turnover and the nascent polypeptide stalling. (B) Volcano plot shows gene pathways upregulated and down regulated in the destabilized mRNA.

FIG. 10. Validation of the LSM10 expression in the destabilized cells across pan cancer models. (A) Bar charts show the ERBB2 expression in the destabilized ERBB2 constructs treated cells in comparison with the controls. (B) Bar charts show the destabilized TEAD1 expression in the treated compared to the controls. (C) Bar charts show the ERBB2 and LSM10 expression in the destabilized ERBB2 constructs treated cells compared to the controls. (D), (E), (F) Bar chart shows the expression of LSM10 in the destabilized cells compared to the controls in the MDAMB231. MDAMB468 and SKOV3 cells, respectively. **p=0.0015, ***p=0.0001, Two tailed T-test.

FIG. 11. The validation of the RPL11 expression in destabilized cells across different cancers. (A) The bar chart shows the destabilized desARE3′UTRERBB2-30 with elevated RPL11 expression compared to the controls. (B), (C), (D) Bar chart shows the high expression of the RPLII in the destabilized cells treated with TEAD1 constructs across different models (MDAMB231, MDAMB468 and SKOV3). **p=0.003, ***p=0.0002, Two tailed T-test.

FIG. 12. Validation of the elevated RPL3 and RPL5 in the destabilized model cells systems. (A) The bar chart shows the RPL3 in the destabilized ERBB2 cells compared to the controls in NCI H1975 (B) The bar charts show elevated levels of RPL3 in the destabilized TEAD1 cells compared to the controls in MDAMB468. (C) The bar charts show elevated levels of RPL3 in the destabilized TEAD1 cells compared to the controls in SKOV3. (D) The bar chart shows the elevated RPL3 in the destabilized c-MYC cells compared to the control in MDAMB231. (E) The bar chart shows the elevated RPL5 in the destabilized c-MYC cells compared to the control in MDAMB231. **p=0.0016, ***p=0.00011, Two tailed T-test.

FIG. 13. Validation of the elevated EXOSC4 in the destabilized model cells systems. (A)-(D) Bar charts show the normalized expression of the EXOSC4 in the destabilized cells of the desARE3′UTRERBB2-30 in NCIH1975, MDAMB468 3′UTRTEAD1, and in 3′UTRMYC1-18 in the MDAMB231, respectively. p=ns=non-significant, * p=0.02, **p=0.0032, ***p=0.00013, Two tailed T-test.

FIG. 14. Validation of the elevated PELO and HBSIL in the destabilized model cells systems. (A)-(B) Bar chart shows the PELO and HBSIL expression in the destabilized model system of 3′UTRTEAD1 and desARE3′UTRERBB2-30, respectively (C)-(E) Bar chart shows the HBSIL expression in the destabilized model system of 3′UTRTEAD1 SKOV3, 3′UTRMYC1-18 MDAMB231, desARE3′UTRERBB2-30 NCIH1975, respectively. *p=0.01, **p=0.004, ***p=0.0001, Two tailed T-test.

FIG. 15. Validation of the elevated ABCE1 in the destabilized model cells systems. (A)-(B) The bar charts show the expression of ABCE1 in the destabilized model cells of MDAMB468 and SKOV3, respectively. *p=0.036, **p=0.002, Two tailed T-test.

FIG. 16. Schematic depiction of the knockout of the ribosome fate molecular switch regulators identified in the destabilized cells. (A) Illustration of the dying destabilized cancer cells bearing destabilizing constructs. (B) Illustration of CRISPR knockout of the EXOSC4, PELO and RPL11. (C) Illustration showing the proliferation of the destabilized dying cells in A. after EXOSC4, PELO and RPL11 have been deleted.

FIG. 17. Schematic depiction of the knockout of the ribosome fate molecular switch regulators identified in the destabilized cells and illustration of gain of function over expression which restored the destabilizing function in the knockout system. (A) Illustration of the dying destabilized cancer cells bearing destabilizing constructs. (B) Illustration of CRISPR knockout of the EXOSC4, PELO and RPL11. © Illustration showing the proliferation of the destabilized dying cells in A. after EXOSC4, PELO and RPL11 have been deleted. (D) Illustration showing over expression vector containing the cDNA of PELO, EXOSC4 and RPL3 transfected into the knocked-out cells in C. (E) Illustration showing the restoration of the destabilized dying cancer cells from the knocked-out cell with the over expressed EXOSC4, PELO and RPL3.

FIG. 18. The morphological features of the EXOSC4 and PELO switch cell fate from degrading to proliferating and from proliferating to degrading under the engineered 3′UTRERBB2-30. (A) Image shows the wildtype NCI H1975 cells. (B) Image shows the NCIH1975 treated with vector. (C) Image shows the NCHI1975 wildtype cells treated with sgRNA non targeting control guides. (D) Image shows the NCIH1975 cells treated with the desARE3′UTRERBB2-30 mRNA destabilizing drug. (E) Image shows the NCIH1975 cells treated with desARE3′UTRERBB2-30 mRNA destabilizing drug then CRISPR KO with sgRNA guide EXOSC4 and PELO. (F) Image shows the desARE3′UTERBB2-30 treated cells CRISPR KO with the sgRNA guides EXOSC4 and PELO and then overexpressed with the cDNA of PELO and EXOSC4. (G) Image shows the desARE3′UTERBB2-30 treated cells CRISPR KO with the sgRNA guides EXOSC4 and PELO and then overexpressed with the cDNA of LSM10 and RPL3. (H) Image shows the desARE3′UTERBB2-30 treated cells CRISPR KO with the sgRNA guides EXOSC4 and PELO and then overexpressed with the cDNA of EXOSC4 and PELO and LSM10 and RPL3.

FIG. 19. Morphology of cells destabilized and CRISPR KO EXOSC4, PELO and RPL11 and OE of EXOSC4, PELO, LSM10, RPL3. (A) Images show the MDAMB231 WT cells. (B) Images show the MDAMB231 cells treated with 3′UTRMYC1-18 mRNA destabilizing drug. (C) Images show the MDAMB231 cells treated with 3′UTRMYC1-18 mRNA destabilizing drug sgRNA non targeting control treated. (D) Images show the MDAMB231 cells treated with 3′UTRMYC1-18 CRISPR KO sgRNA EXOSC4, RPL11 and PELO. (E) Images show the MDAMB231 cells treated with 3′UTRMYC1-18 CRISPR KO sgRNA EXOSC4, LSM10 and RPL11. (F) Images show the MDAMB231 WT cells overexpressed with EXOSC4 and PELO cDNA. (G) Images show the MDAMB231 WT cells overexpressed with LSM10 and RPL3 cDNA. (H) Images show the MDAMB231 WT cells overexpressed with EXOSC4, PELO, LSM10 and RPL3 cDNA.

FIG. 20. Expression profile of the identified ribosome fate switch regulators in the normal healthy cardiomyocytes cells treated with the destabilizing constructs. (A) Bar charts show the expression of MYC, LSM10, RPL3, MRT04 and EXOSC4 in the wild type AC16 WT and AC16 treated with 3′UTRMYC1-18. (B) Bar charts show the expression of ERBB2, LSM10, RPL3, MRT04 and EXOSC4 in the wild type AC16 WT and AC16 treated with the desAR3′UTRERBB2-30.

FIG. 21. Conceptual mechanistic framework to identify molecular regulators of ribosome fate switch controlled by the 3′UTR destabilized mRNA. (A) An illustration of a cell with the destabilized construct making elevated levels of over expressed destabilized mRNA, represented in question mark the unknown ribosomal turnover proteins, the unknown ribosome molecular switch represented as light switch and question mark, and the unknown RNA exosomes marked with Pac-man and question mark. (B) The illustration of destabilized 3′UTR mRNA with multiple premature stops which stalls the translating ribosome and triggers a No-go decay (NGD) or the nonsense mediated decay (NMD). (C) The illustration of in frame premature stop for translating ribosome, which stalls the ribosome, and which leads to in frame diosomes, and multiple stop signals as well on the 3′UTR trigger no go decay/nonsense mediated decay. (D) The illustration of the NGD protein PELO/HBSIL at stalled ribosome and helicase ABCE1 and NEMF/HEL2 triggered cleaving of the ribosomes. (E) The illustration of RNA exosomes EXOSC4 which degrades destabilized mRNA. (F) The illustration of the nascent polypeptide degron mediated by RPL3, 4 and 5. (G) The illustration of the unknown ribosome turnover and synthesis protein mediating remarkably elevated level of the destabilized mRNA alongside the mRNA de-capping promoter DCP1A.

FIG. 22. Graphical illustration of ribosomal functions and regulation. (A) Iron oxide nanocage-destabilized 3′UTR mRNA complex illustrated. (B) The complex uptake by the clathrin-caveolin endocytic machinery. (C) Entry into the cytoplasm as endosome. (D) Endosomal escape releases the IO-nanocage destabilized mRNA complex. The destabilized mRNA gained access to the nucleus via nuclear pores. (E) The destabilized mRNA (light gray bar with explosion signs 14 points-marks destabilized mRNA sequences) in the nucleus specifically recognizes its target mRNA (brown, blue) (F) Multiple recognition sites (3 and above) in frame stalling with ribosomes stalled on destabilized mRNA which triggers a no go decay or nonsense mediated decay leading to PELO, NEMF and ABCE1/HBSIL expression and the RNA exosome EXOSC4 to degrade the destabilized transcript. (G) Depiction of copious number of degraded transcripts from multiple recognition sites in-frame stalling. (H) Two recognition sites in frame stalling with ribosomes stalled on destabilized mRNA which triggers a no go decay or nonsense mediated decay leading to PELO, NEMF and ABCE1/HBSIL expression and the RNA exosome EXOSC4 to degrade the destabilized transcript. (I) Depiction of moderate number of degraded transcripts from the two recognition sites in-frame stalling. (J) 3′UTR recognition sites in 3′UTR stalling with ribosomes stalled on destabilized mRNA which triggers a no go decay or nonsense mediated decay leading to PELO, NEMF and ABCE1/HBSIL expression and the RNA exosome EXOSC4 to degrade the destabilized transcript. (K) Depiction of dwindled number of degraded transcripts from the 3′UTR recognition sites stalling. (L) No 5′UTR recognition sites stalling involved. (M) No transcript degradation.

FIG. 23. RNA structure of the MYCN mRNA destabilizing drug and the determination of the IC₅₀ across different MYCN driven childhood cancers. (A) RNA secondary structure of the 3′UTRMYCN drug with its formula, molecular weight, and base length. (B) Drug dose response curve showing 3′UTRMYCN in comparison with standard of care drugs in rhabdomyosarcoma RD cells. (C) Drug dose response curve showing 3′UTRMYCN in comparison with standard of care drugs in Neuroblastoma SKNBE2 cells. (D) Drug dose response curve showing 3′UTRMYCN in comparison with standard of care drugs in Neuroblastoma Kelly cells.

FIG. 24. The MYCN mRNA destabilizing drugs downregulate MYCN and its interacting partners in a dose dependent manner across different MYCN driven childhood cancers. (A) The bar chart shows the dose dependent downregulation of the MYCN expression by 3′UTRMYCNM1-18 normalized against GAPDH in the neuroblastoma cell lines SKNBE2 compared with the controls. (B) The bar charts show the dose dependent downregulation of the TERT mRNA expression normalized against GAPDH in the neuroblastoma cells lines SKNBE2 treated with 3′UTRMYCNM1-18 in comparison with the controls. (C) The bar charts show the dose dependent downregulation of the EZH2 mRNA expression in the neuroblastoma cells SKNBE2 treated with 3′UTRMYCNM1-14 compared to the controls. (D) The bar chart shows the dose dependent down regulation of the MYCN mRNA expression normalized against the GAPDH in the rhabdomyosarcoma cells RD treated with 3′UTRMYCNM1-14 compared to the controls. p=ns (non-significant), *p=0.011, ***p=0.0021, ****p<0.0001, Two tailed T-test.

FIG. 25. In vivo validation of the MYCN mRNA destabilizing drugs in the inhibition of metastatic rhabdomyosarcoma and improvement of survival outcomes. (A) The Kaplan Meier curve shows the survival outcome of the tumor bearing mice treated with the vector+nanocage, the 3′UTRMYCNM1-18, the 3′UTRMYCNM1-14 treated animals (****P<0.0001) (N=3 vector+nanocage, N=4 3′UTRMYCNM1-18, N=3 3′UTRMYCNM1-14). (B) The graph shows the tumor volume of the tumor bearing animals treated with vector+nanocage, 3′UTRMYCNM1-18 and 3′UTRMYCNM1-14 (N=3 vector+nanocage, N=4 3′UTRMYCNM1-18, N=3 3′UTRMYCNM1-14). (C) The bar chart shows the weight of the tumor bearing animals treated with vector+nanocage, 3′UTRMYCNM1-18 and 3′UTRMYCNM1-14 (N=3 vector+nanocage, N=4 3′UTRMYCNM1-18, N=3 3′UTRMYCNM1-14). p=ns (non-significant), *p=0.01, **p=0.002, ***p=0.0004, ****p<0.0001, Two tailed T-test.

FIG. 26. MYCN mRNA destabilizing drugs inhibit the rhabdomyosarcoma in vivo with complete pathological response. (A) Images show H&E, MYCN and MYOD1 IHC staining of tumor 1 treated with vector+nanocage (N=3 vector+nanocage), the red and green staining shows nuclear expression of MYCN and MYOD1, respectively. (B) Images show H&E, MYCN and MYOD1 IHC staining of tumor 2 treated with vector+nanocage (N=3 vector+nanocage). (C) Images show H&E, MYCN and MYOD1 IHC staining of tumor 2 treated with vector+nanocage (N=3 vector+nanocage). (D) Images show H&E, MYCN and MYOD1 IHC staining of tumor 1 treated with 3′UTRMYCNM1-18 (N=3 3′UTRMYCNM1-18), the red and green staining shows nuclear expression of MYCN and MYOD1, respectively. (E) Images show H&E, MYCN and MYOD1 IHC staining of tumor 2 treated with 3′UTRMYCNM1-18 (N=3 3′UTRMYCNM1-18). (F) Images show H&E, MYCN and MYOD1 IHC staining of tumor 3 treated with 3′UTRMYCNM1-18 (N=3 3′UTRMYCNM1-18). (G) Images show H&E, MYCN and MYOD1 IHC staining of tumor 1 treated with 3′UTRMYCNM1-14 (N=3 3′UTRMYCNM1-14), the red and green staining shows nuclear expression of MYCN and MYOD1, respectively. (H) Images show H&E, MYCN and MYOD1 IHC staining of tumor 2 treated with 3′UTRMYCNM1-14 (N=3 3′UTRMYCNM1-14). (I) Images show H&E, MYCN and MYOD1 IHC staining of tumor 3 treated with 3′UTRMYCNM1-14 (N=3 3′UTRMYCNM1-14). (J) Bar charts show the quantification of the number of malignant pleomorphic hyperchromatic cells in the tumors from the vector+nanocage, the 3′UTRMYCNM1-18 and the 3′UTRMYCNM1-14 treated groups (N=3 vector+nanocage (N=3 3′UTRMYCNM1-18, N=3 3′UTRMYCNM1-14). (K) Bar charts show the percentage of complete pathological responses in the tumors from the vector+nanocage, the 3′UTRMYCNM1-18 and the 3′UTRMYCNM1-14 treated groups. (N=3 vector+nanocage, N=3 3′UTRMYCNM1-18, N=3 3′UTRMYCNM1-14). (L) Bar chart shows the MYCN expression by IHC in the tumors treated with the vector+nanocage, the 3′UTRMYCNM1-18 and the 3′UTRMYCNM1-14 treated groups. (N=3 vector+nanocage, N=3 3′UTRMYCNM1-18, N=3 3′UTRMYCNM1-14). (M) The bar chart shows the MYOD1 expression by IHC in the tumors treated with the vector+nanocage, the 3′UTRMYCNM1-18 and the 3′UTRMYCNM1-14 treated groups. (N=3 vector+nanocage, N=3 3′UTRMYCNM1-18, N=3 3′UTRMYCNM1-14). p=ns (non-significant), ***p=0.00025, Two tailed T-test

FIG. 27. The 3′UTRMYCN mRNA destabilizing drugs inhibited lung metastasis in vivo in the metastatic rhabdomyosarcoma. (A) Images show H&E, MYCN and MYOD1 IHC staining of lung 1 treated with vector+nanocage. The lung parenchyma is filled with hemorrhagic cells (N=3 vector+nanocage). (B) Images show H&E, MYCN and MYOD1 IHC staining of lung 2 treated with vector+nanocage. The lung parenchyma is filled with hemorrhagic cells (N=3 vector+nanocage). (C) Images show H&E, MYCN and MYOD1 IHC staining of lung 3 treated with vector+nanocage. The lung parenchyma is filled with hemorrhagic cells (N=3 vector+nanocage). (D) Images show H&E, MYCN and MYOD1 IHC staining of lung 1 treated with 3′UTRMYCNM1-18 (N=3). (E) Images show H&E, MYCN and MYOD1 IHC staining of lung 2 treated with 3′UTRMYCNM1-18 (N=3). (F) Images show H&E, MYCN and MYOD1 IHC staining of lung 3 treated with 3′UTRMYCNM1-18 (N=3). (G) Images show H&E, MYCN and MYOD1 IHC staining of lung 1 treated with 3′UTRMYCNM1-14 (N=3). (H) Images show H&E, MYCN and MYOD1 IHC staining of lung 2 treated with 3′UTRMYCNM1-14 (N=3). (I) Images show H&E, MYCN and MYOD1 IHC staining of lung 3 treated with 3′UTRMYCNM1-14 (N=3). (J) Bar charts show the quantification of the number of hemorrhagic eosinophilic cells in the lungs parenchyma from the vector+nanocage, the 3′UTRMYCNM1-18 and the 3′UTRMYCNM1-14 treated groups (N=3 vector+nanocage, N=3 3′UTRMYCNM1-18, N=3 3′UTRMYCNM1-14). (K) Bar charts show the quantification of the number of malignant pleomorphic hyperchromatic cells in the lungs parenchyma from the vector+nanocage, the 3′UTRMYCNM1-18 and the 3′UTRMYCNM1-14 treated groups. (N=3 vector+nanocage, N=3 3′UTRMYCNM1-18, N=3 3′UTRMYCNM1-14). (L) Bar charts show the quantification of the lungs parenchyma preservation from the vector+nanocage, the 3′UTRMYCNM1-18 and the 3′UTRMYCNM1-14 treated groups (N=3 vector+nanocage, N=3 3′UTRMYCNM1-18, N=3 3′UTRMYCNM1-14). (M) Bar chart shows the quantification of the MYCN expression by IHC in the vector+nanocage, the 3′UTRMYCNM1-18 and the 3′UTRMYCNM1-14 treated groups (N=3 vector+nanocage, N=3 3′UTRMYCNM1-18, N=3 3′UTRMYCNM1-14). (N) Bar chart shows the quantification of the MYOD1 expression by IHC in the vector+nanocage, the 3′UTRMYCNM1-18 and the 3′UTRMYCNM1-14 treated groups (N=3 vector+nanocage, N=3 3′UTRMYCNM1-18, N=3 3′UTRMYCNM1-14). p=ns (non-significant), *p=0.03, **p=0.001, ***p=0.00018, Two tailed T-test.

FIG. 28. The 3′UTRMYCN mRNA destabilizing drugs inhibited liver metastasis in vivo in the metastatic rhabdomyosarcoma. (A) The images show H&E, MYCN and MYOD1 IHC staining of liver 1 treated with vector+nanocage (N=3 vector+nanocage). (B) The images show H&E, MYCN and MYOD1 IHC staining of liver 2 treated with vector+nanocage (N=3 vector+nanocage). (C) The images show H&E, MYCN and MYOD1 IHC staining of liver 3 treated with vector+nanocage (N=3 vector+nanocage). The arrow shows metastasis to the liver. (D) The images show H&E, MYCN and MYOD1 IHC staining of liver 1 treated with 3′UTRMYCNM1-18 (N=3 3′UTRMYCNM1-18). (E) The images show H&E, MYCN and MYOD1 IHC staining of liver 2 treated with 3′UTRMYCNM1-18 (N=3 3′UTRMYCNM1-18). (F) The images show H&E, MYCN and MYOD1 IHC staining of liver 3 treated with 3′UTRMYCNM1-18 (N=3 3′UTRMYCNM1-18). (G) The images show H&E, MYCN and MYOD1 IHC staining of liver 1 treated with 3′UTRMYCNM1-14 (N=3 3′UTRMYCNM1-14). (H) The images show H&E, MYCN and MYOD1 IHC staining of liver 2 treated with 3′UTRMYCNM1-14 (N=3 3′UTRMYCNM1-14). (I) The images show H&E, MYCN and MYOD1 IHC staining of liver 3 treated with 3′UTRMYCNM1-14 (N=3 3′UTRMYCNM1-14). (J) The bar chart shows the percentage of liver metastasis in the tumor bearing mice treated with vector+nanocage (N=3), 3′UTRMYCNM1-18 (N=3) and 3′UTRMYCNM1-14 (N=3). (K) The bar chart shows the percentage of liver metastasis in the tumor bearing mice treated with vector+nanocage (N=3), 3′UTRMYCNM1-18 (N=3) and 3′UTRMYCNM1-14 (N=3). (L) The bar chart shows the percentage of liver parenchyma preservation in the tumor bearing mice treated with vector+nanocage (N=3), 3′UTRMYCNM1-18 (N=3) and 3′UTRMYCNM1-14 (N=3). (M) Bar chart shows the quantification of the MYCN in the livers of the tumor bearing mice treated with vector+nanocage (N=3), 3′UTRMYCNM1-18 (N=3) and 3′UTRMYCNM1-14 (N=3). (N) Bar chart shows the quantification of the MYOD1 in the livers of the tumor bearing mice treated with vector+nanocage (N=3), 3′UTRMYCNM1-18 (N=3) and 3′UTRMYCNM1-14 (N=3). *p=0.02, **p=0.002, ***p=0.00031, Two tailed T-test.

FIG. 29. The safety profile of the MYCN mRNA destabilizing drugs for blood. (A) The bar chart shows the red blood cell count in the tumor bearing animals treated with vector+nanocage (N=3), 3′UTRMYCNM1-18 (N=4) and 3′UTRMYCNM1-14 (N=3) compared to the healthy non tumor bearing mice (N=2). (B) The bar chart shows the hemoglobin levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (C) The bar chart shows the percentage hematocrit in the tumor bearing animals treated with vector+nanocage (N=3), 3′UTRMYCM1-18 (N=4) and 3′UTRMYCNM1-14 (N=3) compared to the healthy non tumor bearing mice (N=2). (D) The bar chart shows the mean corpuscular hemoglobin levels in the tumor bearing animals treated with vector+nanocage (N=3), 3′UTRMYCNM1-18 (N=4) and 3′UTRMYCNM1-14 (N=3) compared to the healthy non tumor bearing mice (N=2). p=ns (non-significant), Two tailed T-test.

FIG. 30. The safety profile of the MYCN mRNA destabilizing drugs for blood cells, reticulocytes, platelets, and blood urea nitrogen levels. (A) The bar chart shows the mean corpuscular hemoglobin concentration levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (B) The bar chart shows the reticulocytes levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (C) The bar chart shows the platelet levels in the tumor bearing animals treated with vector+nanocage (N=3), 3′UTRMYCM1-18 (N=4) and 3′UTRMYCNM1-14 (N=3) compared to the healthy non tumor bearing mice (N=2). (D) The bar chart shows the blood urea nitrogen levels in the tumor bearing animals treated with vector+nanocage (N=3), 3′UTRMYCNM1-18 (N=4) and 3′UTRMYCNM1-14 (N=3) compared to the healthy non tumor bearing mice (N=2). p=ns (non-significant), Two tailed T-test.

FIG. 31. The safety profile of the MYCN mRNA destabilizing drugs in the kidney function and liver enzymes (ALP-alkaline phosphatase, ALT-alanine transaminase, and AST-aspartate amino transferase). (A) The bar chart shows the creatinine levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (B) The bar chart shows the ALP levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (C) The bar chart shows the ALT levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (D) The bar chart shows the AST levels in the tumor bearing animals treated with vector+nanocage (N=3), 3′UTRMYCNM1-18 (N=4) and 3′UTRMYCNM1-14 (N=3) compared to the healthy non tumor bearing mice (N=2). p=ns (non-significant), **p=0.001, Two tailed T-test.

FIG. 32. The safety profile of the MYCN mRNA destabilizing drugs on the total protein, albumin, globulin, and glucose levels. (A) The bar chart shows the total protein levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (B) The bar chart shows the albumin levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (C) The bar chart shows the globulin levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (D) The bar chart shows the glucose levels in the tumor bearing animals treated with vector+nanocage (N=3), 3′UTRMYCNM1-18 (N=4) and 3′UTRMYCNM1-14 (N=3) compared to the healthy non tumor bearing mice (N=2). p=ns (non-significant), ***p=0.00033, Two tailed T-test.

FIG. 33. The safety profile of the MYCN mRNA destabilizing drugs on the cholesterol and electrolyte levels. (A) The bar chart shows the cholesterol levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (B) The bar chart shows the sodium levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (C) The bar chart shows the calcium levels in the tumor bearing animals treated with vector+nanocage (N=3), 3′UTRMYCNM1-18 (N=4) and 3′UTRMYCNM1-14 (N=3) compared to the healthy non tumor bearing mice (N=2). (D) The bar chart shows the potassium levels in the tumor bearing animals treated with vector+nanocage (N=3), 3′UTRMYCNM1-18 (N=4) and 3′UTRMYCNM1-14 (N=3) compared to the healthy non tumor bearing mice (N=2). p=ns (non-significant), ***p=0.00015, Two tailed T-test

FIG. 34. Identification of the MYCN poly U 3′UTR element and the design of the destabilized MYCN 3′UTR mRNA destabilizing drug. (A) The box shows the MYCN 3′UTR sequence with the stable poly U sequences marked in red. (B) The box shows the position of the stable MYCN 3′UTR sequence changed to destabilized elements marked in green. (C) The box shows the engineered destabilized MYCN 3′UTR mRNA that passed the synthetic g-block test. The sequence in red shows the minimal DCP1aA promoter and the nucleotides in the black are the engineered destabilized 3′UTR MYCN mRNA. The gel picture shows the synthetic destabilized 3′UTRMYCN with red asterisk amplified by the Gibson assembly approach. (D) The gel picture shows the positive clones 14 and 18 of the destabilized 3′UTR MYCN mRNA amplified by the PCR marked with black asterisks. (E) Sanger sequencing shows clones M1-14 and M1-18 mapped to the cloned DCP1A promoter used as the driver of the engineered destabilized 3′UTRMYCN mRNA.

FIG. 35. The effects of the 3′UTRMYCN drugs on the viability of the androgen independent prostate cancer cells PC3 in comparison to standard of care drugs and the migration ability of neuroblastoma cells SKNBE2. (A) The graph shows the drug response curve of 3′UTRMYCNM1-18 in comparison with the standard of care drugs Epirubicin, Cisplatin, Paclitaxel, Olaparib, enzalutamide and abiraterone in PC3. (B) The graph shows the distance of the wound healing in the SKNBE2 cells treated with the 3′UTRMYCNM1-14 drug in a dose dependent manner (40 μg-2.5 μg). (C) The bar chart shows the viability of the AC16 cells treated with the 3′UTRMYCNM1-18 drug in different doses. (D) The bar chart shows the viability of the AC16 cells treated with the 3′UTRMYCNM1-14 drug in a dose dependent manner. p=ns (non-significant), *p=0.04, **p=0.001, ***p=0.0004, Two tailed T-test.

FIG. 36. Schematic of the in vivo animal experiment and the dosing schedule. The schematic shows 10 million RD cells implanted in the thigh muscles after 26 days they engrafted and on day 27, they were randomized into vector+nanocage, 3′UTRMYCNM1-18+nanocage, 3′UTRMYCNM1-14+nanocage groups. Animals were dosed 2λ/week until day 43, after which blood was collected for the safety and toxicity analysis. After day 43, animals were dosed IX/week until day 50, a dosing break of 25 days was observed after which the vector+nanocage treated group and the 3′UTRMYCNM1-18 treated group died on days 62 and 74, respectively. 3′UTRMYCNM1-14 was dosed 2λ/week from day 74 until end of the experiment on day 84.

FIG. 37. The gross pathology image of the lungs from the vector+nanocage treated group and the mRNA destabilizing drug treated groups. The image shows the gross pathology of the lungs from the vector+nanocage, 3′UTRMYCNM1-18 and the 3′UTRMYCNM1-14 treated groups.

FIG. 38. Histological profile of the brains of the rhabdomyosarcoma tumor bearing mice. (A) Images show the H&E staining of the brain of the tumor bearing mice treated with the vector+nanocages (N=2). (B) Images show the H&E staining of the brain of the tumor bearing mice treated with 3′UTRMYCNM1-18+nanocage (N=2). (C) Images show the H&E staining of the brain of the tumor bearing mice treated with 3′UTRMYCNM1-14+nanocage (N=2).

FIG. 39. The p53 status of the tumors from the vector+nanocage treated group and the 3′UTRMYCN treated groups. (A-C) Images show the p53 expression by IHC in the 3 tumors from the vector+nanocage groups. The arrow points to high levels of p53 staining (D-F) Images show the p53 expression by IHC in the 3 tumors from the 3′UTRMYCNM1-18 treated group. (G-I) Images show the p53 expression by IHC in the 3 tumors treated with 3′UTRMYCNM1-14. (J) Bar chart shows the quantification of the p53 expression in the tumors treated with the vector+nanocage (N=3), 3′UTRMYCNM1-18 (N=3) and 3′UTRMYCNM1-14 (N=3) ***p=0.0001, Two tailed T-test.

FIG. 40. IC₅₀ determination of 3′UTRMYC1-18 and standard of care drugs across c-MYC driven TNBC cancers. (A) Dose response drug curve of 3′UTRMYC1-18 in TNBC (MDAMB468) in comparison with the standard of care drugs. (B) Dose response drug curve of 3′UTRMYC1-18 in TNBC (MDAMB231) in comparison with the standard of care drugs.

FIG. 41. IC₅₀ dose combination of 3′UTRMYC1-18 and the standard of care drugs for triple negative breast cancer. The bar charts show viability of the MDAMB231 cells on the IC₅₀ of 3′UTRMYC1-18 and the standard of care drugs alone and in combination.

FIG. 42. Dose dependent down regulation of the MYC mRNA in MDAMB231 and MDAMB468. (A) The bar chart shows the MYC mRNA expression normalized against the GAPDH in the MDAMB231 treated with the dose dependent 3′UTRMYC1-18. (B) The bar chart shows the MYC mRNA expression normalized against the GAPDH in the MDAMB468 treated with the dose dependent 3′UTRMYC1-18

FIG. 43. RNA seq showing 3′UTRMYC1-18 downregulation of MYC. (A) The heat map shows the global gene expression in the MDAMB468 WT, vector and 3′UTRMYC1-18 treated cells. (B) The heat map shows the down regulation of the MYC and its partners in 3′UTRMYC1-18 treated cells compared to the controls.

FIG. 44. Safety of 3′UTRMYC1-18 in the normal cardiomyocytes and epithelial cells. (A) Dose response curve of 3′UTRMYC1-18 and standard of care drugs and MYCi975 in AC16. (B) Dose response curve of 3′UTRMYC1-18 and standard of care drugs and MYCi975 in AC16. (C) Bar chart shows MYC expression in AC16 cells treated in dose dependent manner with 3′UTRMYC1-18. (D) Bar chart shows MYC expression in RWPE1 cells treated in dose dependent manner with 3′UTRMYC1-18.

FIG. 45. MYC mRNA and protein expression in normal healthy cells treated with 3′UTRMYC1-18. (A) Western blot of c-MYC and cellular morphology in normal epithelial cells RWPE1 treated with construct and controls. (B) Images show the healthy RWPE1 cells treated with vector and 3′UTRMYC1-18. (C) Bar charts show the validation of c-MYC expression in the controls and construct treated RWPE1 cells. (D) Global mRNA expression profile of c-MYC and its interactors in the WT and dose dependent 3′UTRMYC1-18 treated RWPE1 cells.

FIG. 46 A. Schematic for dose dependent titration of 3′UTRMYC1-18 in mice with triple negative breast cancer (MDAMB231). The schematic chart depicts 10 million MDAMB231 implanted in the female NSG mice mammary fat pad. After 31 days, the tumors engrafted and on day 32, we randomized the tumor bearing mice into 5 groups according tumor size. The groups were 1) vector+nanocage, 2) 3′UTRMYC1-18 IC₅₀ (8.76 μg), 3) 3′UTRMYC1-18 2×IC₅₀ (17.5 μg), 4) 3′UTRMYC1-18 4×IC₅₀ (35 μg), 5) 3′UTRMYC1-18 IC₅₀ (8.76 μg)+IC₅₀ olaparib (9.5 μg) 6) Healthy non tumor bearing mice treated with 3′UTRMYC1-18 (8.76 μg). Dosing was 2× per week until day 48. On day 48, blood was collected for safety and toxicity profile analysis. Dosing continued 1×/per week until day 55. We stopped between day 55 to 91. Only the 2×IC₅₀ group received dose 1×/2 weeks on day 63 to make up for a dose that was missed between day 48-55. On day 63, the vector+nanocage treated group exceed tumor volume 2.5 cm³ and was euthanized according to protocol. On day 71, the IC₅₀ treated group exceeded the tumor volume 2.5 cm³ was euthanized according to protocol. The 2× and 3×IC₅₀ treated groups and the combination group did not reach this tumor volume of 2.5 cm³. On day 77, blood cells were collected for safety and toxicity profile analysis. On day 91, the healthy non tumor bearing mice that received the IC₅₀ 3′UTRMYC1-18 were euthanized according to protocol.

FIG. 46B. Schematic for dose dependent titration of 3′UTRMYC1-18 in mice with triple negative breast cancer (MDAMB468). The schematic chart depicts that the 10 million MDAMB468 implanted in the female NSG mice mammary fat pad. After 18 days, the tumors were engrafted. On day 18, we randomized the tumor bearing mice into 4 groups according tumor size: 1) vector+nanocage, 2) 3′UTRMYC1-18 IC₅₀ (2.5 μg), 3) 3′UTRMYC1-18 2×IC₅₀ (5 μg), 4) 3′UTRMYC1-18 4×IC₅₀ (10 μg). Dosing by IV was 2× per week until day 18. We took a 32-day break from dosing. On day 52, we resumed dosing 2× per week until day 57. We continued dosing 1× per week until day 71 days. The animals were euthanized according to protocol.

FIG. 47. 3′UTRMYC1-18 inhibits TNBC tumors in vivo in a titratable dose dependent manner. (A) The chart shows the tumor volumes (mm³) for different groups of tumors treated with the vector+nanocage (dark blue), 3′UTRMYC1-18 IC₅₀ (orange), 3′UTRMYC1-18 2×IC₅₀ (green), 3′UTRMYC1-18 4×IC₅₀ (light blue), 3′UTRMYC1-18 IC₅₀+Olaparib IC₅₀ (Purple). *P=001, ***P<0.001, ****P<0.0001, *****P<0.00001. (B) Image of tumors from the different the different dose dependent treatment groups and the control group.

FIG. 47 (con′t). Daily tumor volume measurement showing TNBC MDAMB468 treatment in vivo in a titratable dose dependent manner. (C) The chart shows the tumor volumes (mm³) for different groups of tumors treated with the vector+nanocage (red), 3′UTRMYC1-18 IC₅₀ (purple), 3′UTRMYC1-18 2×IC₅₀ (green), 3′UTRMYC1-18 4× IC₅₀ (light blue), **P=0.001, ****P<0.0001. (D) Image of two tumors from different groups bearing MDAMB468 TNBC treated with the vector+nanocage, 3′UTRMYC1-18 IC₅₀, 3′UTRMYC1-18 2×IC₅₀, 3′UTRMYC1-18 4×IC₅₀. (E) Image of tumors from the vector+nanocage treatment (6 tumors) and 2×IC50 3′UTRMYC1-18 treatment (8 tumors).

FIG. 48. 3′UTRMYC1-18 inhibits TNBC tumors in vivo. (A) Chart showing tumor volumes (mm³) for tumor-bearing TNBC groups treated with 1) vector+nanocage (dark blue), 2) 3′UTRMYC1-18 IC₅₀ (orange), 3) 3′UTRMYC1-18 2×IC₅₀ (green), 4) 3′UTRMYC1-18 4×IC₅₀ (light blue), or 5) 3′UTRMYC1-18 IC₅₀+olaparib IC₅₀ (purple). *P=001, ***P<0.001, ****P<0.0001, *****P<0.00001.

FIG. 48 (con't). 3′UTRMYC1-18 inhibits the TNBC MDAMB468 tumors in vivo in a titratable dose dependent manner. (B) Bar chart shows the quantification of the tumor volume from the vector+nanocage treatment, IC₅₀ treatment, 2×IC₅₀ treatment and 4× IC₅₀ treatment with 3′UTRMYC1-18. *P=0.05, **P=0.001, ***P=0.0001. (C) Bar chart shows the quantification of the tumor volume from vector+nanocage treatment (6 tumors) and 2× IC₅₀ 3′UTRMYC1-18 treatment (8 tumors). ****P<0.0001.

FIG. 49. Kaplan Meier survival curves of the treated groups and the control group. Kaplan-Meier survival curve showing the survival difference between the control group and the various dose dependent treated groups (****P<0.0001).

FIG. 50. H&E staining of the tumors treated with 3′UTRMYC1-18 in a dose dependent manner. (A) Images show H&E staining of tumors from the vector+nanocage treated group (N=3). (B) Images show H&E staining of tumors from the IC₅₀ 3′UTRMYC1-18 (8.76 μg) treated groups (N=3). (C) Images show H&E staining of tumors from the IC₅₀ 3′UTRMYC1-18 (17.52 μg) treated groups (N=3). (D) Images showing H&E staining of tumors from the IC₅₀ 3′UTRMYC1-18 (35 μg) treated groups (N=3). (E) Bar charts show the quantification of the malignant pleomorphic hyperchromatic cell per tumor field in the control and treated groups. (F) Bar charts show the percentage of the complete pathological response in the control and treated groups. (G) Bar charts show the percentage of the partial response in the control and treated groups

FIG. 51. c-MYC IHC staining of tumors treated with 3′UTRMYC1-18 or vector+nanocage. (A) Images show c-MYC staining of the positive control tumors. (B) Images show c-MYC staining of the vector+nanocage treated tumors (N=3). (C) Images show c-MYC staining of the IC₅₀ 3′UTRMYC1-18 (8.7 μg) treated tumors (N=3). (D) Images show c-MYC staining of the 2×IC₅₀ 3′UTRMYC1-18 (17.5 μg) treated tumors (N=3). (E) Images show c-MYC staining of the 4×IC₅₀ 3′UTRMYC1-18 (35 μg) treated tumors (N=3). (F) Bar charts show c-MYC quantification in the control and treated groups.

FIG. 52. Dose dependent downregulation of PD-L1 in 3′UTRMYC1-18 treated mice. (A) Images show PD-L1 staining of the positive control tumors. (B) Images show PD-L1 staining of the vector+nanocage treated tumors (N=3). (C) Images show PD-L1 staining of the IC₅₀ 3′UTRMYC1-18 (8.7 μg) treated tumors (N=3). (D) Images show PD-L1 staining of the 2×IC₅₀ 3′UTRMYC1-18 (17.5 μg) treated tumors (N=3). (E) Images show PD-L1 staining of the 4×IC₅₀ 3′UTRMYC1-18 (35 μg) treated tumors (N=3). (F) Bar charts show PD-L1 quantification in the control and treated groups.

FIG. 53. Inhibition of lung metastasis by 3′UTRMYC1-18. The images show fresh lungs of the tumor bearing mice treated with 3′UTRMYC1-18 in dose dependent manner alone and in combination with Olaparib and the controls.

FIG. 54. Dose dependent inhibition of the lung metastasis by 3′UTRMYC1-18. (A) Images show H&E staining of lungs from the vector+nanocage treated group (N=3). (B) Images show H&E staining of lungs from the IC₅₀ 3′UTRMYC1-18 (8.76 μg) treated groups (N=3). (C) Images show H&E staining of lungs from the IC₅₀ 3′UTRMYC1-18 (17.52 μg) treated groups (N=3). (D) Images show H&E staining of lungs from the IC₅₀ 3′UTRMYC1-18 (35 μg) treated groups (N=4). (E) Bar charts show the quantification of the malignant pleomorphic hyperchromatic cell per lung field in the control and treated groups. (F) Bar charts show the percentage of the lung architecture preservation in the control and treated groups. (G) Bar charts show the percentage of the lung metastasis inhibition in the control and treated groups.

FIG. 55. Dose dependent inhibition of c-MYC in the lungs of mice treated with 3′UTRMYC1-18. (A) Images show c-MYC staining of the positive control lungs. (B) Images show c-MYC staining of the vector+nanocage treated lungs (N=3). (C) Images show c-MYC staining of the IC₅₀ 3′UTRMYC1-18 (8.7 μg) treated lungs (N=3). (D) Images show c-MYC staining of the 2×IC₅₀ 3′UTRMYC1-18 (17.5 μg) treated lungs (N=3). (E) Images show c-MYC staining of the 4×IC₅₀ 3′UTRMYC1-18 (35 μg) treated lungs (N=4). (F) Bar charts show c-MYC quantification in the control and treated groups.

FIG. 56. Dose dependent downregulation of the PD-L1 expression in the lungs of mice treated with 3′UTRMYC1-18. (A) Images show PD-L1 staining of the positive control tissue. (B) Images show PD-L1 staining of the vector+nanocage treated lungs (N=3). (C) Images show PD-L1 staining of the IC₅₀ 3′UTRMYC1-18 (8.7 μg) treated lungs (N=3). (D) Images show PD-L1 staining of the 2×IC₅₀ 3′UTRMYC1-18 (17.5 μg) treated lungs (N=3). (E) Images show PD-L1 staining of the 4×IC₅₀ 3′UTRMYC1-18 (35 μg) treated lungs (N=4). (F) Bar charts show PD-L1 quantification in the control and treated groups.

FIG. 57. Dose dependent inhibition of liver metastasis by 3′UTRMYC1-18. (A) Images show H&E staining of livers from the vector+nanocage treated group (N=3). (B) Images show H&E staining of livers from the IC₅₀ 3′UTRMYC1-18 (8.76 μg) treated groups (N=3). (C) Images show H&E staining of livers from the IC₅₀ 3′UTRMYC1-18 (17.52 μg) treated groups (N=3). (D) Images show H&E staining of livers from the IC₅₀ 3′UTRMYC1-18 (35 μg) treated groups (N=4). (E) Bar charts show the quantification of the malignant pleomorphic hyperchromatic cell per liver field in the control and treated groups. (F) Bar charts show the percentage of the liver metastasis inhibition in the control and treated groups. (G) Bar charts show the percentage of the liver architecture preservation in the control and treated groups.

FIG. 58. Dose dependent inhibition of c-MYC expression in the livers of 3′UTRMYC1-18 and control treatment groups. (A) Images show c-MYC staining of the positive control livers. (B) Images show c-MYC staining of the vector+nanocage treated livers (N=3). (C) Images show c-MYC staining of the IC₅₀ 3′UTRMYC1-18 (8.7 μg) treated livers (N=3). (D) Images show c-MYC staining of the 2×IC₅₀ 3′UTRMYC1-18 (17.5 μg) treated livers (N=3). (E) Images show c-MYC staining of the 4×IC₅₀ 3′UTRMYC1-18 (35 μg) treated livers (N=3). (F) Bar charts show c-MYC quantification in the control and treated groups.

FIG. 59. Dose dependent inhibition of brain metastasis by 3′UTRMYC1-18. (A) Images show H&E staining of brains from the vector+nanocage treated group (N=3), arrow indicates metastasis to the brain. (B) Images show H&E staining of brains from the IC₅₀ 3′UTRMYC1-18 (8.76 μg) treated groups (N=3), arrow indicates metastasis to the brain. (C) Images show H&E staining of brains from the IC₅₀ 3′UTRMYC1-18 (17.52 μg) treated groups (N=3), arrow indicates metastasis to the brain. (D) Images show H&E staining of brains from the IC₅₀ 3′UTRMYC1-18 (35 μg) treated groups (N=3), arrow indicates metastasis to the brain. (E) Bar charts show the percentage of the brain metastasis inhibition in the control and treated groups.

FIG. 60. Inhibition of the c-MYC expression by 3′UTRMYC1-18 in the brain. (A) Images show c-MYC staining of the positive control brain tissue. (B) Images show c-MYC staining of the vector+nanocage treated brain tissue (N=3). (C) Images show c-MYC staining of the IC₅₀ 3′UTRMYC1-18 (8.7 μg) treated brain tissue (N=3). (D) Images show c-MYC staining of the 2×IC₅₀ 3′UTRMYC1-18 (17.5 μg) treated brain tissue (N=3). (E) Images show c-MYC staining of the 4×IC₅₀ 3′UTRMYC1-18 (35 μg) treated brain tissue (N=3). (F) Bar charts show c-MYC quantification in the control and treated groups.

FIG. 61. The pharmacokinetics of 3′UTRMYC1-18 in tumor bearing mice. Chart shows the time dependent absorbance of 3′UTRMYC-18+IO nanocage in the serum of the tumor bearing mice.

FIG. 62. Daily weight measurement of 3′UTRMYC1-18 treated tumor bearing mice and healthy non tumor bearing mice. The graph shows the daily weight of the control, the 3′UTRMYC1-18 treated tumor bearing mice, and the healthy non tumor bearing mice.

FIG. 63. Profile of the red blood cells and hemoglobin levels in the TNBC and healthy mice treated with 3′UTRMYC1-18 in dose dependent manner for short-term and long-term periods. (A) Bar chart shows the red blood cell count in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the red blood cell count in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the hemoglobin levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the hemoglobin levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days.

FIG. 64. Profile of the hematocrit levels and the mean corpuscular volume in TNBC and healthy mice treated with 3′UTRMYC1-18 in dose dependent manner for short-term and long-term periods. (A) Bar chart shows the hematocrit level in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the hematocrit levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the mean corpuscular volume in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the mean corpuscular volume in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days.

FIG. 65. Profile of the mean corpuscular hemoglobin and the mean corpuscular hemoglobin concentration in TNBC and healthy mice treated with 3′UTRMYC1-18 in dose dependent manner for short-term and long-term periods. (A) Bar chart shows the mean corpuscular hemoglobin levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the mean corpuscular hemoglobin levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the mean corpuscular hemoglobin concentration in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the mean corpuscular hemoglobin concentration in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days.

FIG. 66. Profile of the reticulocyte levels and the platelet count in the TNBC and healthy mice treated with 3′UTRMYC1-18 in a dose dependent manner for short-term and long-term periods. (A) Bar chart shows the reticulocyte levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the reticulocytes levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the platelet counts in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the platelet counts in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days.

FIG. 67. Profile of the blood urea nitrogen levels and the creatinine in TNBC and healthy mice treated with 3′UTRMYC1-18 in a dose dependent manner for short-term and long-term periods. (A) Bar chart shows the blood urea nitrogen levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the blood urea nitrogen levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the creatinine levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the creatinine levels in the healthy, non-treated, non-tumor bearing, the 2×IC₅₀ 3′UTRMYC1-18 treated tumor bearing mice and in the healthy non tumor bearing mice treated with the IC₅₀3′UTRMYC1-18 for 45 days.

FIG. 68. Profile of the AST (aspartate aminotransferase) levels and the ALP (alkaline phosphatase) in the TNBC bearing and the healthy mice treated with 3′UTRMYC1-18 in a dose dependent manner for short-term and long-term periods. (A) Bar chart shows the AST levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the AST in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the ALP levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the ALP levels in the healthy, non-treated, non-tumor bearing, the 2×IC₅₀ 3′UTRMYC1-18 treated tumor bearing mice and in the healthy non tumor bearing mice treated with the IC₅₀3′UTRMYC1-18 for 45 days.

FIG. 69. Profile of the ALT (alanine aminotransferase) levels and the total bilirubin in the TNBC bearing and the healthy mice treated with 3′UTRMYC1-18 in a dose dependent manner for short-term and long-term periods. (A) Bar chart shows the ALT levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the ALT in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the total bilirubin levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the total bilirubin levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days.

FIG. 70. Profile of the total protein levels and the albumin levels in the TNBC bearing and the healthy mice treated with 3′UTRMYC1-18 in a dose dependent manner for both short-term and long-term period. (A) Bar chart shows the total proteins levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the total proteins in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the albumin levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the albumin levels in the healthy, non-treated, non-tumor bearing, the 2×IC₅₀ 3′UTRMYC1-18 treated tumor bearing mice and in the healthy non tumor bearing mice treated with the IC₅₀3′UTRMYC1-18 for 45 days.

FIG. 71. Profile of the globulin levels and the glucose levels in the TNBC bearing and the healthy mice treated with 3′UTRMYC1-18 in a dose dependent manner for short-term and long-term periods. (A) Bar chart shows the globulin levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the globulin levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the glucose levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the glucose levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days.

FIG. 72. Profile of the cholesterol levels and the sodium levels in the TNBC bearing and the healthy mice treated with 3′UTRMYC1-18 in a dose dependent manner for short-term and long-term periods. (A) Bar chart shows the cholesterol levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the cholesterol levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the sodium levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days.

FIG. 73. Profile of the potassium levels and the calcium levels in the TNBC bearing and the healthy mice treated with 3′UTRMYC1-18 in a dose dependent manner for short-term and long-term periods. (A) Bar chart shows the potassium levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (B) The bar chart shows the potassium levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days. (C) Bar chart shows the calcium levels in the healthy, non-treated, non-tumor bearing, the vector+nanocage treated tumor bearing mice, the IC₅₀, the 2×IC₅₀, and the 4×IC₅₀ 3′UTRMYC1-18 treated tumor bearing mice and in the healthy non tumor bearing mice treated with the IC₅₀ 3′UTRMYC1-18 for 16 days. (D) The bar chart shows the calcium levels in the healthy, non-treated, non-tumor bearing, the 2×IC₅₀ 3′UTRMYC1-18 treated tumor bearing mice and in the healthy non tumor bearing mice treated with the IC₅₀ 3′UTRMYC1-18 for 45 days.

FIG. 74. Determination of the IC₅₀ of the MYC mRNA 3′UTRMYC1-18 and standard of care drugs and mRNA binding profiles and combination dose. (A) The drug dose response curve of 3′UTRMYC1-18 in a head-to-head comparison with the standard of care drugs and MYC-Max inhibitor in PSN1. (B) The drug dose response curve of 3′UTRMYC1-18 in a head-to-head comparison with the standard of care drugs and MYC-Max inhibitor in MIA-Paca-2. (C) The drug dose response curve of 3′UTRMYC1-18 in a head-to-head comparison with the standard of care drugs and MYC-Max inhibitor in PANC-1. (D) The bar charts show dose dependent downregulation of the c-MYC mRNA expression in MIA-Paca-2 by 3′UTRMYC1-18. (E) The bar chart shows the viability of MIA-Paca-2 cells under the IC₅₀ dose of 3′UTRMYC1-18 and standard of care drugs alone and in combination normalized against the wild type control. ****p<0.00001, ***p=0.0013, **p=0.024, Two tailed T-test.

FIG. 75. In vivo validation of 3′UTRMYC1-18 inhibition of lethal pancreatic cancer. (A) The schematic shows 10 million PSN1 cells implanted in the flank of the NSG mice. after 7 days they engrafted and on day 10, they were randomized into vector+nanocage (21 μg), vector+nanocage (3.6 μg), 3×IC₅₀ 3′UTRMYC1-18+nanocage (3.6 μg), 6×IC₅₀ 3′UTRMYC1-18+nanocage, and 9×IC₅₀ 3′UTRMYC1-18 groups. Animals were dosed 2λ/week until day 32 in the first experiment, blood was collected for the safety and toxicity analysis on day 25. After day 28, animals were dosed 1λ/week until day 33 in 6× and 9×IC₅₀ and no dosing in the 9×IC₅₀ dose until end on day 55. (B) The chart shows the daily tumor volume measurement of the vector+nanocage (#1-red), vector+nanocage (#2-black), 3×IC₅₀ 3′UTRMYC1-18 (orange), 6×IC₅₀ 3′UTRMYC1-18 (green), 9×IC₅₀ 3′UTRMYC1-18 (purple). (C) Kaplan Meier chart shows the survival of tumor bearing animals treated with the vector+nanocage and the dose dependent 3′UTRMYC1-18 treatment. ***P<0.0001. (D) Images of two tumors from the vector+nanocage, the 3×IC₅₀, 6×IC₅₀ and 9×IC₅₀ 3′UTRMYC1-18 dose treatment. ****p<0.00001, ***p=0.0001, *p=0.024, *p=0.044, Two tailed T-test.

FIG. 76. The MYC mRNA destabilizing drug down regulates c-MYC and PD-L1 expression and inhibits pancreatic cancer. (A) Images show H&E, c-MYC and PD-L1 IHC staining of tumor 1 treated with vector+nanocage (N=3 vector+nanocage). (B) Images show H&E, c-MYC and PD-L1 IHC staining of tumor 2 treated with vector+nanocage (N=3 vector+nanocage). (C) Images show H&E, c-MYC and PD-L1 IHC staining of tumor 3 treated with vector+nanocage (N=3 vector+nanocage). (D) Images show H&E, c-MYC and PD-L1 IHC staining of tumor 1 treated with 3×IC₅₀ 3′UTRMYC1-18 (N=3). (E) Images show H&E, c-MYC and PD-L1 IHC staining of tumor 2 treated with 3×IC₅₀ 3′UTRMYC1-18 (N=3). (F) Images show H&E, c-MYC and PD-L1 IHC staining of tumor 3 treated with 3×IC₅₀ 3′UTRMYC1-18 (N=3). (G) Images show H&E, c-MYC and PD-L1 TIC staining of tumor 1 treated with 6×IC₅₀ 3′UTRMYC1-18. (N=3). (H) Images show H&E, c-MYC and PD-L1 IHC staining of tumor 2 treated with 6×IC₅₀ 3′UTRMYC1-18 (N=3). (I) Images show H&E, c-MYC and PD-L1 IHC staining of tumor 3 treated with 6×IC₅₀ 3′UTRMYC1-18 (N=3). (J) Images show H&E, c-MYC and PD-L1 IHC staining of tumor 1 treated with 9×IC₅₀ 3′UTRMYC1-18 (N=3). (K) Images show H&E, c-MYC and PD-L1 TIC staining of tumor 2 treated with 9×IC₅₀ 3′UTRMYC1-18 (N=3). (L) Images show the H&E, c-MYC and PD-L1 IHC staining of tumor 3 treated with 9×IC₅₀ 3′UTRMYC1-18 (N=3). (M) Bar charts show the quantification of the number of malignant pleomorphic hyperchromatic cells in the tumors from the vector+nanocage the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). (N) Bar charts show the percentage of complete pathological responses in the tumors from the vector+nanocage, the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). (O) Bar chart shows the c-MYC expression by IHC in the positive control and in the tumors treated with the vector+nanocage, the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). (P) The bar chart shows the PD-L1 expression by IHC in the positive control and in the tumors treated with the vector+nanocage, the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). ****p<0.00001, ***p=0.0002, **p=0.0012, p=ns (non-significant), Two tailed T-test.

FIG. 77. 3′UTRMYC1-18 inhibits liver metastasis in pancreatic cancer and down regulates c-MYC and PD-L1 expression. (A) Images show H&E, c-MYC and PD-L1 IHC staining of liver 1 treated with vector+nanocage (N=3 vector+nanocage). (B) Images show H&E, c-MYC and PD-L1 IHC staining of liver 2 treated with vector+nanocage (N=3 vector+nanocage); hemorrhagic lesions marked with arrow. (C) Images show H&E, c-MYC and PD-L1 IHC staining of liver 3 treated with vector+nanocage (N=3 vector+nanocage); hemorrhagic lesions marked with arrow. (D) Images show H&E, c-MYC and PD-L1 IHC staining of liver 1 treated with 3×IC₅₀ 3′UTRMYC1-18. (N=3). (E) Images show H&E, c-MYC and PD-L1 IHC staining of liver 2 treated with 3×IC₅₀ 3′UTRMYC1-18 (N=3); hemorrhagic lesions marked with arrow. (F) Images show H&E, c-MYC and PD-L1 IHC staining of liver 3 treated with 3×IC₅₀ 3′UTRMYC1-18 (N=3). (G) Images show H&E, c-MYC and PD-L1 IHC staining of liver 1 treated with 6×IC₅₀ 3′UTRMYC1-18 (N=3). (H) Images show H&E, c-MYC and PD-L1 IHC staining of liver 2 treated with 6×IC₅₀ 3′UTRMYC1-18 (N=3). (I) Images show H&E, c-MYC and PD-L1 IHC staining of liver 3 treated with 6×IC₅₀ 3′UTRMYC1-18 (N=3). (J) Images show H&E, c-MYC and PD-L1 IHC staining of liver 1 treated with 9×IC₅₀ 3′UTRMYC1-18 (N=3). (K) Images show H&E, c-MYC and PD-L1 IHC staining of liver 2 treated with 9×IC₅₀ 3′UTRMYC1-18 (N=3). (L) Images show H&E, c-MYC and PD-L1 IHC staining of liver 3 treated with 9×IC₅₀ 3′UTRMYC1-18 (N=3). (M) Bar charts show the quantification of the number of malignant pleomorphic hyperchromatic cells in the livers from the vector+nanocage the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). (N) Bar charts show the percentage of hemorrhagic lesions in the livers from the vector+nanocage, the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). (O) Bar chart shows the c-MYC expression by IHC in the positive control and in the livers treated with the vector+nanocage, the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). (P) The bar chart shows PD-L1 expression by IHC in the positive control, in the vector+nanocage treated group, and in the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). ****p<0.000025, ***p=0.00018, **p=0.004, p=ns (non-significant), Two tailed T-test.

FIG. 78. The inhibition of lung metastasis in pancreatic cancer and down regulation of c-MYC by the c-MYC mRNA destabilizing drug. (A) Images show H&E, c-MYC and PD-L1 IHC staining of lung 1 treated with vector+nanocage (N=3 vector+nanocage); hemorrhagic lesions marked with arrow. (B) Images show H&E, c-MYC and PD-L1 IHC staining of lung 2 treated with vector+nanocage. (N=3 vector+nanocage); hemorrhagic lesion marked with arrow. (C) Images show H&E, c-MYC and PD-L1 IHC staining of lung 3 treated with vector+nanocage (N=3 vector+nanocage); hemorrhagic lesion marked with arrow. (D) Images show H&E, c-MYC and PD-L1 IHC staining of lung 1 treated with 3×IC₅₀ 3′UTRMYC1-18 (N=3); hemorrhagic lesion marked with arrow. (E) Images show H&E, c-MYC and PD-L1 IHC staining of lung 2 treated with 3×IC₅₀ 3′UTRMYC1-18 (N=3); hemorrhagic lesions marked with arrow. (F) Images show H&E, c-MYC and PD-L1 IHC staining of lung 3 treated with 3×IC₅₀ 3′UTRMYC1-18 (N=3); hemorrhagic lesions marked with arrow. (G) Images show H&E, c-MYC and PD-L1 IHC staining of lung 1 treated with 6×IC₅₀ 3′UTRMYC1-18 (N=3). (H) Images show H&E, c-MYC and PD-L1 IHC staining of lung 2 treated with 6×IC₅₀ 3′UTRMYC1-18 (N=3). (I) Images show H&E, c-MYC and PD-L1 IHC staining of lung 3 treated with 6×IC₅₀ 3′UTRMYC1-18 (N=3). (J) Images show H&E, c-MYC and PD-L1 IHC staining of lung 1 treated with 9×IC₅₀ 3′UTRMYC1-18 (N=3). (K) Images show H&E, c-MYC and PD-L1 IHC staining of lung 2 treated with 9×IC₅₀ 3′UTRMYC1-18 (N=3). (L) Images show H&E, c-MYC and PD-L1 IHC staining of lung 3 treated with 9×IC₅₀ 3′UTRMYC1-18 (N=3). (M) Bar charts show the quantification of the number of malignant pleomorphic hyperchromatic cells in the lungs from the vector+nanocage the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). (N) Bar charts show the percentage of hemorrhagic lesions in the lungs from the vector+nanocage, the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). (O) Bar chart shows the c-MYC expression by IHC in the positive control and in the lungs treated with the vector+nanocage, the 3×IC₅₀, the 6×IC₅₀, the 9×IC₅₀ 3′UTRMYC1-18 treated groups (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). (P) The bar chart shows PD-L1 expression by IHC in the positive control and in the lungs treated with the vector+nanocage, the 3×IC₅₀, the 6×IC₅₀, the 9×IC₅₀ 3′UTRMYC1-18 treated groups. (N=3 vector+nanocage, N=3 3×IC₅₀ 3′UTRMYC1-18, N=3 6×IC₅₀ 3′UTRMYC1-18, N=3 9×IC₅₀ 3′UTRMYC1-18). p=ns (non-significant), ****p<0.000105, **p=0.002, Two tailed T-test.

FIG. 79. The safety profile of 3′UTRMYC1-18 on the red blood cells and kidney function in pancreatic cancer tumor bearing mice and controls. (A) The bar chart shows the red blood cell count in the healthy non tumor bearing mice and, in the tumor, bearing animals treated with the vector+nanocage (N=2) and the 3×IC₅₀ 3′UTRMYC1-18 (N=2). (B) The bar chart shows the hemoglobin levels in the healthy non tumor bearing mice and, in the tumor, bearing animals treated with the vector+nanocage (N=2) and the 3×IC₅₀ 3′UTRMYC1-18 (N=2). (C) The bar chart shows the blood urea nitrogen levels in the healthy non tumor bearing mice and, in the tumor, bearing animals treated with the vector+nanocage (N=2) and the 3×IC₅₀ 3′UTRMYC1-18 (N=2). (D) The bar chart shows the creatinine levels in the healthy non tumor bearing mice and, in the tumor, bearing animals treated with the vector+nanocage (N=2) and the 3×IC₅₀ 3′UTRMYC1-18 (N=2). p=ns (non-significant), Two tailed T-test.

FIG. 80. The safety profile of 3′UTRMYC1-18 on the liver enzyme ALP, total protein, and albumin in pancreatic cancer tumor bearing mice and controls. (A) The bar chart shows the ALP (alkaline phosphatase) levels in the healthy non tumor bearing mice and, in the tumor, bearing animals treated with the vector+nanocage (N=2) and the 3×IC₅₀ 3′UTRMYC1-18 (N=2). (B) The bar chart shows the total protein levels in the healthy non tumor bearing mice and, in the tumor, bearing animals treated with the vector+nanocage (N=2) and the 3×IC₅₀ 3′UTRMYC1-18 (N=2). (C) The bar chart shows the albumin levels in the healthy non tumor bearing mice and, in the tumor, bearing animals treated with the vector+nanocage (N=2) and the 3×IC₅₀ 3′UTRMYC1-18 (N=2). (D) The bar chart shows the globulin levels in the healthy non tumor bearing mice and, in the tumor, bearing animals treated with the vector+nanocage (N=2) and the 3×IC₅₀ 3′UTRMYC1-18 (N=2). **p=0.023, p=ns (non-significant), Two tailed T-test.

FIG. 81. The safety profile of 3′UTRMYC1-18 on the pancreatic function glucose, and cholesterol and electrolytes in pancreatic cancer tumor bearing mice and controls. (A) The bar chart shows the glucose levels in the healthy non tumor bearing mice and, in the tumor, bearing animals treated with the vector+nanocage (N=2) and the 3×IC₅₀ 3′UTRMYC1-18 (N=2). (B) The bar chart shows the cholesterol levels in the healthy non tumor bearing mice and, in the tumor, bearing animals treated with the vector+nanocage (N=2) and the 3×IC₅₀ 3′UTRMYC1-18 (N=2). (C) The bar chart shows the sodium levels in the healthy non tumor bearing mice and, in the tumor, bearing animals treated with the vector+nanocage (N=2) and the 3×IC₅₀ 3′UTRMYC1-18 (N=2). (D) The bar chart shows the potassium levels in the healthy non tumor bearing mice and, in the tumor, bearing animals treated with the vector+nanocage (N=2) and the 3×IC₅₀3′UTRMYC1-18 (N=2). p=ns (non-significant), Two tailed T-test.

FIG. 82. Daily weight recording of the pancreatic cancer treated mice and controls. Chart shows the daily weight recording of the tumor bearing mice treated with the vector+nanocage, 3×IC₅₀, 6×IC₅₀ and 9×IC₅₀ 3′UTRMYC1-18 doses.

FIG. 83. H&E images of the brain of the pancreatic cancer treated mice and controls. (A) H&E images of brain 1-3 from the vector+nanocage treated group with the brain metastasis lesion marked with arrow. (B) H&E images of brain 1-3 from the 3×IC₅₀ 3′UTRMYC1-18 treated group with the brain metastasis lesion marked with arrow. (C) H&E images of brain 1-3 from the 6×IC₅₀ 3′UTRMYC1-18 treated group. (D) H&E images of brain 1-3 from the 9×IC₅₀ 3′UTRMYC1-18 treated group. (E) The bar chart shows the quantification of the brain metastasis in brains of tumor bearing mice treated with the vector+nanocage, 3×IC₅₀, 6×IC₅₀ and 9×IC₅₀ 3′UTRMYC1-18 dose. p=ns (non-significant), ****p<0.0003, Two tailed T-test.

FIG. 84. Dose dependent inhibition of the primary ovarian cancer cells and cell lines and c-MYC and migration ability of the ovarian cancer cells by 3′UTRMYC1-18. (A) Drug dose response curve of 3′UTRMYC1-18 in ovarian cancer cells A2780 in a head-to-head comparison with the standard of care drugs and MYCi975 (MYC-Max inhibitor). (B) Drug dose response curve of 3′UTRMYC1-18 in the primary ovarian cancer cells, P5X in a head-to-head comparison with the standard of care drugs. (C) Drug dose response curve of 3′UTRMYC1-18 in the primary ovarian cancer cells, the OCI-9× in a head-to-head comparison with the standard of care drugs. (D) The bar chart shows the dose dependent downregulation of the MYC mRNA by 3′UTRMYC1-18 in a A2780 ovarian cancer cells. (E) The bar chart shows migration of the A2780 cancer cells treated with 3′UTRMYC1-18, vector or the WT controls for 4 days. (F) The bar chart shows the viability of the ovarian cancer cells, OVCAR8 cells treated with 3′UTRMYC1-18 and the control vector.

FIG. 85. In vivo validation of ovarian cancer cell inhibition by 3′UTRMYC1-18. (A) Schematic depiction of the in vivo experiments. 10 million P5X primary ovarian cancer cells were implanted orthotopically into the ovaries of the female NSG mice. After 35 days, the tumors were engrafted. On day 36, animals were randomized into vector+nanocage and IC₅₀ 3′UTRMYC1-18 treatment groups. The animals were dosed 2×/week. After 77 days, the controls vector+nanocage group exceeded tumor volume died. The IC₅₀ 3′UTRMYC1-18 treated group lived to 86 days. (B) The chart shows the daily tumor volumes of the tumors treated with vector+nanocage and IC₅₀ 3′UTRMYC-18. (C) The Kaplan Meier survival curves for the vector+nanocage and IC₅₀ 3′UTRMYC1-18 treated groups. (D) The images show H&E staining of tumors from the vector+nanocage treated animals. (E) The images show H&E staining of the tumors from IC₅₀ 3′UTRMYC1-18 treated animals.

FIG. 86. 3′UTRMYC1-18 inhibits ovarian cancer and down regulates c-MYC-PD-L1-Pax8-p21. (A) The images show ovary tumor 1 from the vector+nanocages treated group with H&E staining, c-MYC, PD-L1, PAX8 and p21 IHC staining. (B) The images show ovary tumor 2 from the vector+nanocages treated group with H&E staining, c-MYC, PD-L1, PAX8 and p21 IHC staining. (C) The images show ovary tumor 3 from the vector+nanocages treated group with H&E staining, c-MYC, PD-L1, PAX8 and p21 IHC staining. (D) The images show ovary tumor 1 from the IC₅₀ 3′UTRMYC1-18 treated group with H&E staining, c-MYC, PD-L1, PAX8 and p21 IHC staining. (E) The images show ovary tumor 2 from the IC₅₀ 3′UTRMYC1-18 treated group with H&E staining, c-MYC, PD-L1, PAX8 and p21 IHC staining. (F) The images show ovary tumor 3 from the IC₅₀ 3′UTRMYC1-18 treated group with H&E staining, c-MYC, PD-L1, PAX8 and p21 IHC staining. (G) Bar charts show the quantification of the number of malignant pleomorphic hyperchromatic cells in the tumors from the vector+nanocage and the IC₅₀ 3′UTRMYC1-18 treated groups. (N=3 vector+nanocage, N=3 IC₅₀ 3′UTRMYC1-18). (H) Bar chart shows the c-MYC expression by IHC in the positive control and in the tumors treated with the vector+nanocage, and the IC₅₀ 3′UTRMYC1-18 treated groups. (N=3 vector+nanocage, N=3 IC₅₀ 3′UTRMYC1-18). (I) The bar chart shows the PD-L1 expression by IHC in the positive control and in the tumors treated with the vector+nanocage and the IC₅₀ 3′UTRMYC1-18 treated groups. (N=3 vector+nanocage, N=3 IC₅₀ 3′UTRMYC1-18). (J) The bar chart shows the PAX8 expression by IHC in the positive control and in the tumors treated with the vector+nanocage and the IC₅₀ 3′UTRMYC1-18 treated groups. (N=3 vector+nanocage, N=3 IC₅₀ 3′UTRMYC1-18). (K) The bar chart shows the p21 expression by IHC in the positive control and in the tumors treated with the vector+nanocage and the IC₅₀ 3′UTRMYC1-18 treated groups. (N=3 vector+nanocage, N=3 IC₅₀ 3′UTRMYC1-18).

FIG. 87. 3′UTRMYC1-18 inhibits liver metastasis from ovarian cancer and down regulates c-MYC-PAX8-p21. (A) The images show liver 1 from the vector+nanocages treated group of the ovarian cancer bearing mice with H&E staining, c-MYC, PD-L1, PAX8 and p21 IHC staining. (B) The images show liver 2 from the vector+nanocages treated group of ovarian cancer bearing mice with H&E staining, c-MYC, PD-L1, PAX8 and p21 IHC staining. (C) The images show liver 3 from the vector+nanocages treated group with of the ovarian cancer bearing mice with H&E staining, c-MYC, PD-L1, PAX8 and p21 IHC staining. (D) The images show liver 1 from the IC₅₀ 3′UTRMYC1-18 treated group of ovarian cancer bearing mice with H&E staining, c-MYC, PD-L1, PAX8 and p21 IHC staining. (E) The images show liver 2 from the IC₅₀ 3′UTRMYC1-18 treated group of ovarian cancer bearing mice with H&E staining, c-MYC, PD-L1, PAX8 and p21 IHC staining. (F) The images show liver 3 from the IC₅₀ 3′UTRMYC1-18 treated group of ovarian cancer bearing mice with H&E staining, c-MYC, PD-L1, PAX8 and p21 IHC staining. (G) Bar charts show the quantification of the number of malignant pleomorphic hyperchromatic cells in the livers from the vector+nanocage and the IC₅₀, 3′UTRMYC1-18 treated groups. (N=3 vector+nanocage, N=3 IC₅₀ 3′UTRMYC1-18). (H) Bar charts show the quantification of the number of metastases in the livers from the vector+nanocage and the IC₅₀, 3′UTRMYC1-18 treated groups. (N=3 Vector+Nanocage, N=3 IC₅₀ 3′UTRMYC1-18). (I) Bar chart shows the c-MYC expression by IHC in the positive control and in the tumors treated with the vector+nanocage, and the IC₅₀ 3′UTRMYC1-18 treated groups. (N=3 vector+nanocage, N=3 IC₅₀ 3′UTRMYC1-18). (J) The bar chart shows the PD-L1 expression by IHC in the positive control and in the tumors treated with the vector+nanocage and the IC₅₀ 3′UTRMYC1-18 treated groups. (N=3 vector+nanocage, N=3 IC₅₀ 3′UTRMYC1-18). (K) The bar chart shows the PAX8 expression by IHC in the positive control and in the tumors treated with the vector+nanocage and the IC₅₀ 3′UTRMYC1-18 treated groups. (N=3 Vector+Nanocage, N=3 IC₅₀ 3′UTRMYC1-18). (L) The bar chart shows the p21 expression by IHC in the positive control and in the tumors treated with the vector+nanocage and the IC₅₀ 3′UTRMYC1-18 treated groups. (N=3 vector+nanocage, N=3 IC₅₀ 3′UTRMYC1-18).

FIG. 88. 3′UTRMYC1-18 inhibits lung metastasis from ovarian cancer and down regulates c-MYC-PAX8. (A) The images show lung 1 from the vector+nanocages treated group of the ovarian cancer bearing mice with H&E staining, c-MYC, PD-L1, PAX8 and p21 IHC staining. (B) The images show lung 2 from the vector+nanocages treated group of ovarian cancer bearing mice with H&E staining, c-MYC, PD-L1, PAX8 and p21 IHC staining. (C) The images show lung 3 from the vector+nanocages treated group of the ovarian cancer bearing mice with H&E staining, c-MYC, PD-L1, PAX8 and p21 IHC staining. (D) The images show lung 1 from the IC50 3′UTRMYC1-18 treated group of ovarian cancer bearing mice with H&E staining, c-MYC, PD-L1, PAX8 and p21 IHC staining. (E) The images show lung 2 from the IC50 3′UTRMYC1-18 treated group of ovarian cancer bearing mice with H&E staining, c-MYC, PD-L1, PAX8 and p21 IHC staining. (F) The images show lung 3 from the IC50 3′UTRMYC1-18 treated group of ovarian cancer bearing mice with H&E staining, c-MYC, PD-L1, PAX8 and p21 IHC staining.

FIG. 89. Quantification of the malignant ovarian cancer cells and complete pathological response. (A) Bar chart shows the quantification of the number of malignant pleomorphic cells per tumor filed in the treated versus the controls (B) Bar chart shows the quantification of the complete pathological response in the control and 3′UTRMYC1-18 treated ovarian cancer. (C) Graph shows the daily weight measurement of the animals from the vector+nanocage and the 3′UTRMYC1-18 IC₅₀ treated groups.

FIG. 90. No differential changes in pathology or c-MYC-PD-L1-PAX8-p21 in the fallopian tubes of the controls and 3′UTRMYC1-18 treated animals. (A) The images show fallopian tube 1 from the vector+nanocages treated group of the ovarian cancer bearing mice with H&E staining, c-MYC, PD-L1 and p21 IHC staining. (B) The images show fallopian tube 2 from the vector+nanocages treated group of ovarian cancer bearing mice with H&E staining, c-MYC, PD-L1 and p21 IHC staining. (C) The images show fallopian tube 3 from the vector+nanocages treated group of the ovarian cancer bearing mice with H&E staining, c-MYC, PD-L1, and p21 IHC staining. (D) The images show fallopian tube 1 from the IC₅₀ 3′UTRMYC1-18 treated group of ovarian cancer bearing mice with H&E staining, c-MYC, PD-L1, and p21 IHC staining. (E) The images show fallopian tube 2 from the IC₅₀ 3′UTRMYC1-18 treated group of ovarian cancer bearing mice with H&E staining, c-MYC, PD-L1, and p21 IHC staining. (F) The images show fallopian tube 3 from the IC₅₀ 3′UTRMYC1-18 treated group of ovarian cancer bearing mice with H&E staining, c-MYC, PD-L1, and p21 IHC staining.

### DETAILED DESCRIPTION

The following description sets forth numerous exemplary configurations, parameters, and the like. It should be recognized, however, that such description is not intended as a limitation on the scope of this disclosure; it is instead provided as exemplary embodiments.

### Definitions

Throughout the specification and claims, the following terms take the meanings explicitly associated herein, unless the context clearly dictates otherwise.

The various examples, embodiments, and aspects as set out herein may be readily combined, without departing from the scope or spirit of this disclosure. Thus, the phrase “in one example”, “in one embodiment”, or “in one aspect” is not necessarily exclusive of other examples, embodiments, or aspects that are also described. In the same way, the phrase “in another example”, “in another embodiment”, or “in another aspect” is not necessarily exclusive of other examples, embodiments, or aspects that are described.

In each instance herein, in descriptions, embodiments, and examples of the present disclosure, the terms “comprising”, “including”, etc, are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

The term “consisting essentially of”, as used herein, refers to an active agent present in a composition. For example, the active agent may be at least 85% by weight of the composition, or at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, at least 99.9%, or approximately 100.0% by weight of the composition (% w/w). For liquid compositions, the active agent may be at least 85% by volume of the composition, or at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, at least 99.9%, or approximately 100.0% by volume of the composition (% v/v).

Where a range is given in the specification, for example, a temperature range, a time range, or a composition range, all intermediate ranges, and subranges, as well as all individual values included in the ranges given are intended to be included in the disclosure. Thus, each range that is specified (e.g., 1 to 10) includes all possible combinations of numerical values between the lowest value and the highest value enumerated (e.g., 1, 1.1, 2, 3, 3.3, 4, 5.5, 6, 7, 8.9, 9 and 10) and also any range of rational numbers within that range (e.g., 2 to 8, 1.5 to 5.5, and 3.1 to 4.9), and, therefore, all sub-ranges of all ranges expressly disclosed herein are hereby expressly disclosed. The numeric values provided in parentheses here are only examples of what is specifically intended and all possible combinations of numerical value between the lowest value and the highest value enumerated are to be considered to be expressly stated in this disclosure in a similar manner.

As used herein “and/or” means additionally or alternatively.

As used herein “based on” is not exclusive and allows for being based on additional factors not described, unless the context clearly dictates otherwise. The meaning of “in” includes “in” and “on.”

Any use of a term in the singular also encompasses plural forms. Thus, throughout the specification, the meaning of “a”, “an”, and “the” include plural references.

The term “about” or “approximately” means up to 10% greater than or up to 10% lesser than a particular value, or up to 5% greater than or up to 5% lesser than a particular value.

The term “polynucleotide(s),” as used herein, means a single or double-stranded deoxyribonucleotide or ribonucleotide polymer of any length, and include as non-limiting examples, coding and non-coding sequences of a gene, genomic DNA, recombinant polynucleotides, isolated and purified naturally occurring DNA or RNA sequences, synthetic RNA and DNA sequences, fragments, variants, derivatives, constructs, and vectors. Reference to nucleic acids, nucleic acid molecules, nucleotide sequences, and polynucleotide sequences is to be similarly understood.

The term “polypeptide”, as used herein, encompasses amino acid chains of any length, wherein the amino acid residues are linked by covalent peptide bonds. “Polypeptide” may refer to a polypeptide that is a purified natural product, or that has been produced partially or wholly using recombinant or synthetic techniques. The term may refer to an aggregate of a polypeptide such as a dimer or other multimer, a fusion polypeptide, a polypeptide fragment, a polypeptide variant, fragment, or derivative thereof. The term “polypeptide” is used interchangeably herein with the terms “protein” and “enzyme”.

A “fragment” of a polynucleotide is a subsequence of a particular nucleic acid molecule, i.e., truncation. The term may refer to a polynucleotide fragment, an aggregate of a polynucleotide fragment, a fusion polynucleotide fragment, a fragment of a polynucleotide variant, or a fragment of a polynucleotide derivative thereof.

As used herein, the term “RNA” relates to a molecule which comprises ribonucleotide residues and preferably being entirely or substantially composed of ribonucleotide residues. The term “ribonucleotide” relates to a nucleotide with a hydroxyl group at the 2′-position of a P-D-ribofuranosyl group. The term “RNA” comprises double stranded RNA, single stranded RNA, isolated RNA such as partially or completely purified RNA, essentially pure RNA, synthetic RNA, and recombinantly generated RNA such as modified RNA, which differs from naturally occurring RNA by addition, deletion, substitution, and/or alteration of one or more nucleotides. Such alterations can include addition of non-nucleotide material, such as to the end(s) of a RNA or internally, for example, at one or more nucleotides of the RNA. Nucleotides in RNA molecules can also comprise non-standard nucleotides, such as non-naturally occurring nucleotides or chemically synthesized nucleotides. These altered RNAs can be referred to as analogs, particularly analogs of naturally-occurring RNAs. As used herein, RNA includes mRNA.

The term “mRNA” means “messenger-RNA” and relates to a transcript that is generated by using a DNA template and encodes a peptide or protein. Typically, mRNA comprises a 5′-UTR, a protein coding region, a 3′-UTR, and a poly(A) sequence. mRNA may be generated by in vitro transcription from a DNA template. The in vitro transcription methodology is known to one skilled in the ail. For example, there are a variety of in vitro transcription kits commercially available. As detailed herein, mRNA can be modified by incorporating various destabilizing motifs into the 3′UTR region (e.g., UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein).

As used herein, the term “MYC” is synonymous with “c-MYC”.

The terms “3′-untranslated region” and “3′LUTR,” as used herein, relate to a region that is located at the 3′ end of an RNA molecule, preferably an mRNA molecule. This region may be downstream of the termination codon of a protein-encoding region. It may be transcribed but not translated into an amino acid sequence. A first polynucleotide region may be considered to be located downstream of a second polynucleotide region, if the 5′ end of the first polynucleotide region is the part of the first polynucleotide region closest to the 3′ end of the second polynucleotide region.

The terms “poly(uridylic acid) sequence,” “poly(U) sequence,” and “poly(U) stabilizing motif” refer to a sequence of uridylic acid residues that are typically located at the 3′ end of an RNA molecule. Generally, the poly(A) sequence at the end of the 3′UTR is important for the nuclear export, translation, and stability of mRNA. The sequence is shortened over time, and, when it is short enough, the mRNA is enzymatically degraded. To counteract poly(A) mediated degradation, poly(U) sequences can interact with poly (A) tails to inhibit the association of poly (A) binding protein and to confer increased stability upon introduction into ectopic transcripts. Poly(U) and poly (A) interactions can prevent negative regulation of mRNA. As detailed herein, in various aspects, a poly(U) sequence can have at least two, at least three, at least 4, at least 5, at least 6, at least 7, at least 8, at least 9, at least 10, or more than ten consecutive uridylic acid residues.

The terms “adenylate-uridylate-rich elements,” “AU-rich elements,” and “AREs” refer to sequences that are rich in adenosine and uridine bases, typically located within the 3′UTR of a RNA molecule. These elements are binding sites for proteins, which proteins, in response to different intracellular and extracellular signals, can promote mRNA decay, effect mRNA stability, or promote translation.

As used herein, the term “reverse transcription” means a process wherein the genetic code in an RNA sequence (e.g., an RNA molecule as described herein) is reverse transcribed into DNA. As used herein, the term “reverse transcription” includes in vitro reverse transcription, which relates to a process wherein DNA, in particular, cDNA, is in vitro synthesized in a cell-free system.

As used herein, the term “vector” means any vehicle for carrying a nucleic acid that can, for example, enable said nucleic acid to be introduced into prokaryotic and/or eukaryotic host cells and, where appropriate, to be integrated into a genome. Such vectors may be replicated and/or expressed in the cell. Vectors may include, for example, plasmids, phagemids, and VIMS genomes.

The term “plasmid,” as used herein, generally relates to a construct of extrachromosomal genetic material, usually a circular DNA duplex, which can replicate independently of chromosomal DNA.

The term “plasmid vector” means a small, single or double-stranded circular extrachromosomal DNA or RNA construct. Plasmid vectors can consist of the transgene insert and an origin of replication, a promoter region, optionally a selectable marker, and convenient restriction sites. The features may allow for semi-independent replication of the plasmid in the host e.g., hundreds of copies can be made per cell) and may provide convenient restriction sites. The term “vector” may refer to double stranded DNA plasmid vectors used to carry DNA encoding for the lentiviral plasmid vector.

The term “expressing” refers to the expression of a nucleic acid transcript from a nucleic acid template and/or the translation of that transcript into a peptide or polypeptide, and is used herein as commonly used in the art.

As used herein, an “isolated” component (e.g., isolated polynucleotide or polypeptide) refers to a component that has been purified from (e.g., separated from) other components. An isolated component may be removed from its originating environment, e.g., natural cellular environment or synthetic environment. The isolated component of this disclosure may be prepared by at least one purification step. An isolated component may have: about 70% purity or greater, about 80% purity or greater, about 90% purity or greater; or, in particular aspects, about 99% purity or greater. An isolated component may be obtained by any method or combination of methods as known and used in the art, including biochemical, recombinant, and synthetic techniques.

“Isolated” when used herein in reference to a cell or host cell describes to a cell or host cell that has been obtained or removed from an organism or from its natural environment or from an artificial environment. The term encompasses single cells, per se, as well as cells or host cells comprised in a cell culture and can include a single cell or single host cell.

“Naturally occurring” as used herein with reference to a polynucleotide or polypeptide sequence refers to a sequence that is found in nature. A synthetic sequence that is identical to a wild type sequence is, for the purposes of this disclosure, considered a naturally occurring sequence. A naturally occurring sequence also refers to a variant sequence as found in nature. These include, for example, allelic variants and naturally occurring sequences due to hybridisation or horizontal gene transfer, and variants arising out of other natural processes. What is important for a naturally occurring sequence is that the actual sequence (e.g., nucleotide or amino acid sequence) is found or known from nature.

“Non-naturally occurring” as used herein with reference to a polynucleotide or polypeptide sequence refers to a sequence that is not found in nature. Examples of non-naturally occurring sequences include artificially produced and variant sequences, made for example by recombination, domain swapping, point mutation, insertion, deletion, or other methods, or combinations of these methods. Non-naturally occurring sequences also include chemically evolved sequences. What is important for a non-naturally occurring sequence is that the actual sequence (e.g., nucleotide or amino acid sequence) is not found or known from nature.

Where this description refers to the utilisation of “non-naturally occurring” molecules, it will be understood that the corresponding “naturally occurring” molecules may also be utilized, if this should be desired. Where the description does not indicate whether molecules are “naturally occurring” or “non-naturally occurring”, then either may be suitably used, unless the context dictates otherwise.

The term “recombinant” refers to a polynucleotide sequence that is removed from sequences that surround it in its natural context and/or is recombined with sequences that are not present in its natural context. A “recombinant” peptide or polypeptide sequence is produced by translation from a “recombinant” polynucleotide sequence.

As used herein, the term “variant” refers to polynucleotide, peptide, or polypeptide sequences different from the specifically identified sequences, wherein one or more nucleotides or amino acid residues is deleted, transposed, substituted, or added. Variants may be naturally occurring allelic variants, or non-naturally occurring variants. Variants may be from the same or from other species and may encompass homologues, paralogues, and orthologues. In certain embodiments, the variants useful in this disclosure have biological activities that are the same or similar to those of a corresponding wild type molecule; i.e., functional variants of the parent polypeptide or polynucleotide. In certain embodiments, the variants have biological activities that differ from their corresponding wild type molecules. In certain embodiments, the differences are altered activity, stability, and/or production levels.

As used herein, the term “mutagenesis” refers to methods to alter a polynucleotide sequence either in vitro or in vivo, most commonly to change the sequence of one or more polypeptides encoded therein. Mutagenesis methods include as non-limiting examples, site-directed mutagenesis, de novo synthesis of sequences carrying mutations, error-prone PCR, DNA shuffling, chemical mutagenesis, application of ultraviolet radiation, genome shuffling, and use of mutator strains.

The term, “wild type” when used herein with reference to a polynucleotide or polynucleotide refers to a naturally occurring, non-mutant form of the molecule. A mutant polynucleotide means a polynucleotide that has sustained a mutation, including one or more of a point mutation, insertion, deletion, substitution, amplification, or translocation, but not limited thereto. A mutant polypeptide means a polypeptide that includes a mutation, including one or more of an insertion, deletion, substitution, but not limited thereto. A wild-type polypeptide may be expressed from a wild-type polynucleotide, or from a mutant polynucleotide.

As used herein, the term in vitro refers to a reaction performed outside of the confines of a living cell or a host organism.

As used herein, the term in vivo refers to a reaction performed within a living cell and/or within a host organism.

As used herein, the term “nanoparticle” refers to any particle having a diameter making the particle suitable for administration to a subject. Noted are systemic forms of administration, and in particular, parenteral administration. The particles may have a diameter of less than 1000 nm, or less than 600 nm, or less than 400 nm.

As used herein, the term “nanoparticulate formulation” or similar term refer to any composition comprising nanoparticles. Such compositions may include, for example, at least 0.2 pg. at least 1.0 pg, at least 5.0 pg, at least 10 pg, at least 15 pg or at least 20 pg of nanoparticles. The nanoparticulate composition may be a uniform collection (e.g., from about 0.2 pg to about 20 pg) of nanoparticles. The nanoparticulate composition may be a dispersion or emulsions.

The terms “lipoplex” and “nucleic acid lipoplex” mean a complex of lipids and nucleic acids. The nucleic acids may include RNA. Lipoplexes can be formed in mixture of cationic liposomes, which may include a neutral helper lipid, and one or more nucleic acids.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to solutions, dispersions, suspensions, or emulsions, as well as powders for reconstitution into injectable solutions or dispersions just prior to use. Various exemplifications are provided herein.

The term “excipient” includes all substances that can be present in a pharmaceutical composition and which are not active ingredients. This can be, for example, an organic or inorganic component, having a natural or non-natural (synthetic) nature, with which the active component is combined. An “excipient” may include one or more compatible solid fillers, liquid fillers, diluents, encapsulating substances, or other compositions which are suitable for administration to a patient.

As used herein, the term “subject” can be a vertebrate, such as a mammal, a fish, a bird, a reptile, or an amphibian. Included are domesticated animals, livestock, and laboratory animals. Specifically included are humans, non-human primates, horses, pigs, rabbits, dogs, sheep, goats, cows, cats, guinea pigs, and rodents. The term does not denote a particular age or sex. Thus, adult and newborn subjects, as well as fetuses, are intended to be included.

A “patient” refers to a subject afflicted with a disease or disorder. The term includes human and non-human subjects (e.g., veterinary patients).

The terms “administering” and “administration” refer to any method of providing a pharmaceutical preparation to a subject. Such methods include, for example, oral administration, transdermal administration, administration by inhalation, nasal administration, and topical administration, amongst others. Various exemplifications are provided herein.

“Co-administration” or “co-administering” refers to the combined use of active components, for example, for therapy or for cosmetic enhancement, and includes the administration of co-formulations (i.e., combination formulations), as well as the simultaneous or sequential administration of separate formulations. Similarly, “in conjunction” refers to the combined use of one or more active components and a device/procedure. This can include use of the active component(s) preceding use of the device/procedure, simultaneously with the device/procedure, and/or following use of the device/procedure.

As disclosed herein, the terms “cancer disease” and “cancer” (medical term: malignant neoplasm) refers to a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor, i.e., a swelling or lesion formed by an abnormal growth of cells (called neoplastic cells or tumor cells), but some, like leukemia, do not. The term “cancer” also includes cancer metastases.

As used herein, the term “treatment” refers to the medical management of a patient with the intent to cure, ameliorate, stabilize, or prevent a disease condition, pathological condition, or disorder. This term includes improvement of a disease condition, pathological condition, or disorder. This term also includes treatment directed toward removal of the cause of the associated disease condition, pathological condition, or disorder. In addition, this term includes relief of symptoms and minimizing or partially or completely inhibiting the development of the associated disease condition, pathological condition, or disorder. This term can also include inhibiting the disease, i.e., arresting its development or relieving the disease, i.e., causing regression of the disease. Also included is treatment employed to supplement another specific therapy directed toward the improvement of the associated disease condition, pathological condition, or disorder.

As used herein, the terms “prevent”/“preventing” refer to precluding, averting, obviating, forestalling, stopping, or hindering something from happening, especially by advance action. This can include preventing the disease from occurring in a subject that can be predisposed to a disease condition, pathological condition, or disorder. This can also include halting or delaying the onset or progression of a disease condition, pathological condition, or disorder. A preventative measure may result in the stoppage or delay of development of the a disease condition, pathological condition, or disorder, or its symptom(s), a prevention of progression of the disease condition, pathological condition, or disorder or its symptom(s), or a lessening of the developed a disease condition, pathological condition, or disorder or its symptom(s) if such happen to arise. A preventative measure may also act in supporting, maintaining, and/or protecting of a bodily system. It should be understood that the term “treating or preventing” does not exclude the possibility of obtaining both treatment and prevention of the disorder. A “therapeutic” effect or “therapeutic” method may include treatment, or prevention, or both.

As used herein, the term “diagnosed” means having been subjected to a physical examination by a person of skill, for example, a physician, and found to have a condition that can be diagnosed or treated by the molecules, vectors, compositions, or methods disclosed herein.

It is understood that, for any DNA molecule disclosed herein, the corresponding RNA molecule and peptide/polypeptide molecules are also encompassed and disclosed. For any RNA molecule disclosed herein, the corresponding DNA and peptide/polypeptide molecules are also encompassed and disclosed. Likewise, for any peptide/polypeptide molecule disclosed herein, the corresponding RNA and DNA sequences are also considered to be encompassed and disclosed. In addition, where there are multiple sequence identifiers, e.g., “SEQ ID NO: 1-3” or “SEQ ID NO: 4-27”, this format may be understood as referring to each sequence individually, or any combination thereof.

### RNA Molecules

The MYC family of oncogenic transcription factors includes c-MYC, MYCN and MYCL, each of which are involved in various forms of cancers (2a). The MYC genes are activated in many cancers via chromosomal translocation, gene amplification, retroviral insertion, and transduction (3a, 4a). c-MYC is a master transcription factor that is over expressed in more than 70% of human cancers. To date, there has been no clinically approved direct inhibitor of c-MYC available. MYCN is a basic helix-loop-helix master E-box transcription factor (1a). Like c-MYC, MYCN has remained an elusive drug target. MYCN is over-expressed in many childhood cancers such as the metastatic resistant neuroblastoma, metastatic rhabdomyosarcoma, Wilms tumor, retinoblastoma and medulloblastoma (5a, 6a, 7a). MYCN is a major oncogenic driver of these cancers' aggressiveness and metastasis.

ERBB2/HER2 is a member of the subclass I receptor tyrosine kinase superfamily of ERBB/EGFR (epidermal growth factor receptor family) which has four members, namely, EGFR/ERBB1, ERBB2, ERBB3, and ERBB4. ERBB2 is activated upon binding of the neuregulin ligand onto the ERBB receptor which leads to its homo- and heterodimerization triggering the activation of tyrosine kinases which have docking sites on the ERBB receptors. These in turn control signalling proteins, transcription factors, and kinases which mediate ERBB functions. ERBB2/ERBB2 is overexpressed in many human cancers including breast, lung, and colorectal cancers. The overexpression of ERBB2 is associated with very aggressive breast and drug-resistant lung cancer because ERBB2 is a membrane protein that signals and amplifies for proliferation, pro-survival, and prometastatic signals of the cancer leading to poor clinical outcomes. ERBB2 is resistant to trastuzumab and other tyrosine kinase inhibitors.

Our first aim was to understand how the engineered destabilized 3′UTR ERBB2, c-MYC, and TEAD1 constructs function in degrading their specific target mRNA. We investigated these constructs and found the upregulation of PELO, EXOSC4, RPL3 and 11 subunit proteins as being implicated in switching ribosome translation to degrading upon recognition of the destabilizing drug for its target transcript. In further analysis, we discovered that the loss of the EXOSC4 and the PELO switches ribosomes from degrading to translating in the presence of the mRNA destabilizing drugs (3′UTR constructs of ERBB2). Conversely, in a gain of function, the overexpression of PELO and EXOSC4 restores the degradation activity of the ribosomes in relation to the target transcript.

Our second aim was to achieve direct targeting of the MYCN mRNA on the MYCN 3′UTR. We found that our 3′UTR MYCN mRNA destabilizing drugs are on-target for MYCN mRNA. We observed inhibition of metastatic rhabdomyosarcoma primary tumors and metastasis to the lungs and liver with complete pathological response. We saw significant survival outcomes and showed that the drugs were safe and well tolerated with no toxicity to the blood cells, liver, kidney, or pancreas. Moreso, we determined that the drugs have relevance for MYCN amplified childhood cancers like Kelly and SKNBE2 neuroblastoma and for androgen independent prostates cancers, like PC3.

Our third aim was to achieve dose-dependent in vivo treatment of various cancers using a 3′UTR MYC mRNA destabilizing drug. We observed highly effective treatments for breast cancers, nervous system cancers, pancreatic cancers, ovarian cancers, and colon cancers, along with the inhibition of metastases. We found that the MYC mRNA destabilizing drug inhibited the tumors in a dose dependent manner, and exhibited a safe and tolerable therapeutic profile in short-and -long term analyses.

Accordingly, this disclosure provides an innovative approach for treating various diseases and disorders associated with dysregulation of ERBB2, MYC, and/or MYCN. Specifically, mRNA constructs have been engineered in which the stabilization ARE motifs have been replaced with destabilized consensus motifs. The disclosed RNA molecules, once reverse transcribed into DNA and transfected into a vector, become integrated into the genome, and then outcompete the endogenous RNA molecules, thereby degrading the target transcript and reducing levels of the corresponding protein.

Exemplary mRNA stabilization and destabilization motifs are shown in the table below.

TABLE A

| Designation | Sequence |
|---|---|
| PolyU stabilization sequence - 2 nt | UU |
| PolyU stabilization sequence - 3 nt | UUU |
| PolyU stabilization sequence - 4 nt | UUUU |
| PolyU stabilization sequence - 5 nt | UUUUU |
| PolyU stabilization sequence - 6 nt | UUUUUU |
| PolyU stabilization sequence - 7 nt | UUUUUUU |
| PolyU stabilization sequence - 8 nt | UUUUUUUU |
| PolyU stabilization sequence - 9 nt | UUUUUUUUU |
| PolyU stabilization sequence - 10 nt | UUUUUUUUUU (SEQ ID NO: 49) |
| Destabilized RNA sequence - 3 nt | UCU |
| Destabilized RNA sequence - 4 nt | AUUU |
| Destabilized RNA sequence - 4 nt | CCUC |
| Destabilized RNA sequence - 4 nt | CUGC |
| Destabilized RNA sequence - 5 nt | AUUUU |
| Destabilized RNA sequence - 5 nt | UUCGU |
| Destabilized RNA sequence - 5 nt | ACCUC |
| Destabilized RNA sequence - 5 nt | CGCGU |
| Destabilized RNA sequence - 6 nt | UGCCUU |
| Destabilized RNA sequence - 7 nt | CCUCUGC |
| Destabilized RNA sequence - 8 nt | UAAGUUAU |
| Destabilized RNA sequence - 8 nt | UAACUUAU |
| Destabilized RNA sequence - 8 nt | GUAAAUAG |
| Destabilized RNA sequence - 9 nt | UAAGUUAUG |
| Destabilized RNA sequence - 9 nt | UGCUGCCCU |
| Destabilized RNA sequence - 10 nt | UCCUGCCCUC (SEQ ID NO: 50) |
| Destabilized RNA sequence - 11 nt | CCUCCUGCUUA (SEQ ID NO: 51) |
| Destabilized RNA sequence - 11 nt | CCUCGUAACUU (SEQ ID NO: 52) |
| Destabilized RNA sequence - 11 nt | CCUCCUGCCUC (SEQ ID NO: 53) |
| Destabilized RNA sequence - 12 nt | CCUCCUGCAUUU (SEQ ID NO: 54) |
| Destabilized RNA sequence - 12 nt | CCUCGCUGCCUC (SEQ ID NO: 55) |
| Destabilized RNA sequence - 14 nt | CUGCUAAGUUAUCU (SEQ ID NO: 56) |
| Destabilized RNA sequence - 16 nt | UAAGUUAUCCUCUAUU (SEQ ID: 57) |
| Destabilized RNA sequence - 17 nt | CUGCCUCUGCUAACUUAU (SEQ ID: 58) |

As detailed herein, the 3′UTR of ERBB2, MYC, or MYCN mRNAs are normally enriched with poly(U) sequences that are stabilizing AU rich elements. Thus, the disclosed RNA molecules of the disclosure have a 3′UTR region in which one or more poly(U) stabilizing motifs have been replaced with a destabilizing motif, such as, for example, UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

In accordance with this disclosure, a poly(U) sequence can contain, for example, at least two consecutive U's, at least three consecutive U's, at least four consecutive U's, at least five consecutive U's, or more than five consecutive U's. The poly(U) sequence may have more than nine consecutive U's, more than 10 consecutive U's, more than 11 consecutive U's, or more than 12 consecutive U's.

The RNA molecule may be destabilized in at least two ARE poly(U) stabilizing motifs of the 3′UTR, at least three ARE poly(U) stabilizing motifs of the 3′UTR, at least four ARE poly(U) stabilizing motifs of the 3′UTR, or at each ARE poly(U) stabilizing motif of the 3′UTR. The RNA may further comprise a polyadenyl sequence that may be located at the 3′ end of the RNA molecule.

Exemplary engineered destabilized mRNA constructs, with destabilization motifs, are shown in the table below (motifs in bold with underlining).

TABLE B

| Designation | Sequence/SEQ ID NO: |
|---|---|
| c-MYC | GAGCAAUCACCUAUGAACUUGCUGCUAAGUUAUGAUCAAAUGCAACCUCAC |
| | AACCUUGGCUGAGUCUUGAGACUGAAAGAUUUAGCCAUAAUGUAAACUGCC |
| | UCAAAUUGGACUUUGGGCAUAAAAGAACCCUCCUGCAUGCUUACCAUCCCU |
| | CCUGCCCUCCUUUAACAGCCUCGUAACUUAUAAUUGACCUCAAAAAACCUC |
| | AAGAUUUACACAAUGUUUCUCUGUAAAUAUUGCCAUUAAAUGUAAAUAACU |
| | UUAAUAAAACGUUUAUAGCAGUUACACAGAAUUUCAAUCCUAGUAUAUAGU |
| | ACCUAGUAUUAUAGGUACUAUAAACCCUAACCUCCUGCAUUUAAGUACAUU |
| | UUGCACUGCAAAGUAAGUUAUCCUCUAUUGACCUCAGAAAAAAUAAAAUAA |
| | CUGGCAAAUAUAUCAUUGAGCCAAAUCUUAAGUUGUGAAUGCUGCCUCUGC |
| | UAACUUAUCCCUCCCAACCACCACCAUCCCUGCUGCCCUCAUCAA (SEQ ID |
| | NO: 1) |
| ERBB2 | CAGGGGAACCUGCCAUGCCAGGAACCUGUCCUAAGGAACCUGCCUUCCUGC |
| | UUGAGUUCCCAGAUGGCUGGAAGGGGUCCAGCCUCGUUGGAAGAGGAACAG |
| | CACUGGGGAGUCUUCGUGGAUUCUGAGGCCCUGCCCAAUGAGACUCUAGGG |
| | UCCAGUGGAUGCCACAGCCCAGCUUGGCCCUCUCCUUCCAGAUCCUGGGUA |
| | CUGAAAGCCUUAGGGAAGCUGGCCUGAGAGGGGAAGCGGCCCUAAGGGAGU |
| | GUCUAAGAACAAAAGCGACCCAUUCAGAGACUGUCCCUGAAACCUAGUACU |
| | GCCCCCCAUGAGGAAGGAACAGCAAUGGUGUCAGUAUCCAGGUCGCGUUCA |
| | GAGUGCCCUCCUGCUUAGCUGCUAAGUUAUCUGCCUCGCUGCCUCAAAGAU |
| | GAAAUAAAGACCCAGGGGGAGAAUGGGUGUUGUAUGGGGAGGCAAGUGUGG |
| | GGGGUCCUUCUCCACACCCACUUUGUCCAUUUGCAAAUAUAU (SEQ ID NO: |
| | 2) |
| MYCN | CCUCCUGCCUCCAAACAAACAUUGUGUUGACAUUAAGAAUGUUGGUUUACU |
| | UUCAAAUCGGUCCCCUGUCGAGUUCGGCUCUGGGUGGGCAGUAGGACCACC |
| | AGUGUGGGGUUCUGCUGGGACCUUGGAGAGCCUGCAUCCCAGGAUGCUGGG |
| | UGGCCCUGCAGCCUCCUCCACCUCACCUCCAUGACAGCGCUAAACGUUGGU |
| | GACGGUUGGGAGCCUCUGGGGCUGUUGAAGUCACCUUGUGUGUUCCAAGUU |
| | UCCAAACAACAGAAAGUCAUUCCUUCCUCUUAAAAUGGUGCUUAAGUUCCA |
| | GCAGAUGCCACAUAAGGGGUUUGCCAUUUGAUACCCCUGGGGAACAUUUCU |
| | GUAAAUACCAUUGACACAUCCGCCUUUUGUAUACAUCCUGGGUAAUGAGAG |
| | GUGGCUUUUGCGGCCAGUAUUAGACUGGAGUUCAUACCUAAGUACUGUAAU |
| | AAUACCUCAAUGUUUGAGGAGCAUGCCUCGUAUACAAAUAUAUUGUUAAUC |
| | UCUGUUAUGUACUGUACUAAUUCUUACACUGCCUGUAUACUUUAGUAUGAC |
| | GCUGAUACAUAACUAAAUUUGAUACUUAUAUUUUCGUAUGAAAAUGAGUUG |
| | UGAAAGUUUUGAGUAGAUAUUACCUCAUCACCUCUUGAACUAAGAAACUUU |
| | UGUAAAGAAAUUUACUAUAUAUAUAUGCCUCUGC (SEQ ID NO: 3) |

The Y-untranslated region typically extends from the termination codon for a translation product to the poly(A) sequence, which is usually attached after the transcription process. The 3′-untranslated regions of mammalian mRNA typically have a homology region known as the AAUAAA hexanucleotide sequence. This sequence is presumably the poly(A) attachment signal and is frequently located from 10 to 30 bases upstream of the poly(A) attachment site.

3′-untranslated regions can contain one or more inverted repeats, which can fold to give stem-loop structures that act as barriers for exoribonucleases or interact with proteins known to increase RNA stability e.g., RNA-binding proteins). The average length of human 3′-untranslated regions is between 800-1000 nucleotides. The length of the 3′UTR is involved in determining both translational efficiency and the stability of an mRNA.

It can be readily determined whether a 3′-untranslated region or a nucleic acid sequence derived therefrom decreases the stability and/or translation efficiency of RNA, by incorporating the 3′-untranslated region or the nucleic acid sequence derived therefrom into the 3′-untranslated region of a mRNA and measuring whether said incorporation decreases the amount of protein synthesized. Methods of determining whether a RNA molecule is destabilized relative to a wildtype RNA molecule are well established. See, e.g., Koh et al. (2019) Scientific reports vol. 9(1): 5976.

The mRNA decay rate can affect steady-state mRNA abundance and mRNA turnover. At any given time in the cell, mRNA is synthesized by polymerases and destroyed by nucleases. When these two events occur at a constant rate, they give rise to a steady-state mRNA population for each unique transcript. Variations in mRNA transcription rates are generally recognized for their central importance in regulating gene expression.

UTRs, particularly the 3′UTR, play a role in transcript expression regulation by controlling mRNA stability, decay, and translation. Transcript stability can be affected by various CA-elements, such as AREs, in the 3′UTR. Thus, in various aspects, the stability of a RNA molecule can be evaluated by measuring the decay rate of the RNA molecule relative to the decay rate of a wildtype RNA molecule comprising a 3′UTR encoding an ERBB2, MYC, or MYCN protein.

Specifically disclosed are ribonucleic acid (RNA) molecules comprising a 3′-untranslated region (3′UTR) of an mRNA encoding an ERBB2, MYC, or MYCN protein, in which one or more adenylate-uridine rich element (ARE) poly(uridylic acid) (poly(U)) stabilizing motifs of the 3′UTR are substituted with a nucleic acid sequence selected from: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

As an exemplification, an RNA molecule can comprise a 3′UTR of an mRNA encoding an ERBB2, MYC, or MYCN protein, in which one or more ARE poly(U) stabilizing motifs of the 3′UTR are substituted with a nucleic acid sequence consisting of or comprising: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

For example, the disclosed RNA molecule can be at least 1%, at least 5%, at least 10%, at least 15%, at least 20%, at least 25%, at least 30%, at least 40%, at least 50%, or more than 50% destabilized compared to wildtype RNA molecules comprising a 3′UTR encoding an ERBB2, MYC, or MYCN protein.

In various aspects, the RNA molecule comprising a 3′UTR of an mRNA encoding an ERBB2, MYC, or MYCN protein, in which one or more ARE poly(U) stabilizing motifs of the 3′UTR are substituted with a nucleic acid sequence consisting of or comprising: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

This can destabilize a wildtype RNA molecule comprising a 3′UTR encoding an ERBB2, MYC, or MYCN protein, such that the destabilized wildtype RNA molecule is at least 1%, at least 5%, at least 10%, at least 15%, at least 20%, at least 25%, at least 30%, at least 40%, at least 50%, or more than 50% destabilized compared to a wildtype RNA molecule that has not been destabilized by a RNA molecule of this disclosure.

In various aspects, the RNA molecule comprises a 3′UTR of an mRNA encoding an ERBB2, MYC, or MYCN protein, in which one or more ARE poly(U) stabilizing motifs of the 3′UTR arc substituted with a nucleic acid sequence consisting of or comprising: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

In various further aspects, the RNA molecule comprises a 3′UTR of an mRNA encoding an ERBB2, MYC, or MYCN protein, in which at least two, at least three, or at least four ARE poly(U) stabilizing motifs of the 3′UTR are substituted with a nucleic acid sequence consisting of or comprising: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

The one or more ARE poly(U) stabilizing motifs may have at least two consecutive U's. The one or more ARE poly(U) stabilizing motifs may have at least three consecutive U's. The one or more ARE poly(U) stabilizing motifs may have at least four consecutive U's.

The destabilizing motif may consist of or comprise the sequence CCUC. The destabilizing motif may consist of or may comprise the sequence CCUCU-S-S, where S is a C or G. The destabilizing motif may consist of or comprise the sequence CCUCCU-S-CCUC (SEQ ID NO: 162), where S is C or G.

In various aspects, the RNA molecule is destabilized in at least two ARE poly(U) stabilizing motifs. In a further aspect, the RNA molecule is destabilized in at least three ARE poly(U) stabilizing motifs. In a still further aspect, the RNA molecule is destabilized in at least four ARE poly(U) stabilizing motifs. In an even further aspect, each ARE poly(U) stabilizing motif is destabilized.

In one aspect, a ribonucleic acid (RNA) molecule is provided comprising a 3′-untranslated region (3′UTR) of an mRNA encoding a MYCN protein, in which one or more adenylate-uridine rich element (ARE) poly(uridylic acid) (poly(U)) stabilizing motifs of the 3′UTR are substituted with a nucleic acid sequence selected consisting of or comprising: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

Accordingly, a RNA molecule of this disclosure may consist of or comprise one or more of: SEQ ID NO: 1-3. Variant RNA molecules are also encompassed (e.g., having one or more nucleotides deleted, added, or transposed).

In specific aspects, the RNA molecule of the disclosure may be prepared to include a 3′UTR in which at least two, at least three, or at least four ARE poly(U) stabilizing motifs of the 3′UTR are substituted with a destabilizing motif to produce a nucleic acid which has at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 1-3.

In specific aspects, the nucleic acid may have at least two, at least three, or at least four destabilizing motifs as described herein (e.g., 100% identity to the disclosed motif(s)) and the remainder of the nucleotide sequence may have at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 1-3.

In other aspects, the 3′UTR of the mRNA encoding an ERBB2, MYC, or MYCN protein is a variant having at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 1-3.

In other aspects, the 3′UTR of the mRNA encoding an ERBB2, MYC, or MYCN protein is a variant having destabilizing motifs as disclosed (e.g., 100% identity to the disclosed motif(s)) and, for the remainder of the nucleotide sequence, having at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 1-3.

For polynucleotides, exemplary sequence alignment platforms include but are not limited to: homology alignment algorithms (Needleman and Wunsch (1970) J Mol Biol 48: 443); local homology algorithms (Smith and Waterman (1981) Adv Appl Math 2: 482); searches for similarity (Pearson and Lipman (1988) PNAS USA 85: 2444). In specific embodiments, the BLAST algorithm may be used (Altschul et al. (1990) J Mol Biol 215: 403-410; Henikoff and Henikoff (1989) PNAS USA 89: 10915; Karlin and Altschul (1993) PNAS USA 90: 5873-5787). Software for performing BLAST analyses is publicly available through the National Center for Biotechnology Information. Other examples of alignment software include GAP, BESTFIT, FASTA, PILEUP, and TFASTA provided by Wisconsin Genetics Software Package (Genetics Computer Group), and CLUSTAL programs such as ClustalW, ClustalX, and Clustal Omega (see, e.g., Thompson et al. (1994) Nuc Acids Res 22: 4673-4680).

### DNA Molecules

This disclosure provides DNA molecules that can be utilized in the preparation of the therapeutic RNA molecules as described herein. In one aspect, disclosed are deoxyribonucleic acid molecules comprising in the 5′ to 3′ direction of transcription relative to synthesis of mRNA molecule comprising a 3′UTR of an mRNA encoding an ERBB2, MYC, or MYCN protein.

In specific aspects, the DNA molecule can include (a) a promoter, operatively linked to (b) a nucleic acid sequence encoding a 3′UTR of an ERBB2, MYC, or MYCN gene etc in the mRNA molecule in which one or more ARE poly(U) stabilizing motifs of the 3′UTR of the mRNA are substituted with a nucleic acid sequence consisting of or comprising: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

As detailed herein, the disclosed DNA molecules can be cloned into a vector for use as a therapeutic agent to target an ERBB2, MYC, or MYCN gene (e.g. oncogene) overexpression. Such overexpression is observed in a variety of diseases and disorders for which dysregulation of ERBB2, MYC, or MYCN is implicated including, but not limited, to various cancers as set out herein.

In various aspects, the disclosed DNA molecules (e.g., a disclosed cDNA molecule) can be prepared by reverse transcribing a disclosed RNA molecule. Thus, in various aspects, disclosed are DNA (e.g., cDNA) molecules prepared from a disclosed RNA molecule. In various further aspects, reverse transcription is carried out in vitro. Through these methods, the ERBB2, MYC, or MYCN 3′UTR can be destabilized. Expression can be driven by a promoter (e.g., a de-capping promoter such as DCP1A) to specifically degrade the transcript and protein through nonsense mediated decay.

Exemplary destabilization sequences are shown in the table below.

TABLE C

| Designation | Sequence |
|---|---|
| DNA sequence for destabilized RNA - 3 nt | TCT |
| DNA sequence for destabilized RNA - 4 nt | ATTT |
| DNA sequence for destabilized RNA - 4 nt | CCTC |
| DNA sequence for destabilized RNA - 4 nt | CTGC |
| DNA sequence for destabilized RNA - 5 nt | ATTTT |
| DNA sequence for destabilized RNA - 5 nt | TTCGT |
| DNA sequence for destabilized RNA - 5 nt | ACCTC |
| DNA sequence for destabilized RNA - 5 nt | CGCGT |
| DNA sequence for destabilized RNA - 6 nt | TGCCTT |
| DNA sequence for destabilized RNA - 7 nt | CCTCTGC |
| DNA sequence for destabilized RNA - 8 nt | TAAGTTAT |
| DNA sequence for destabilized RNA - 8 nt | TAACTTAT |
| DNA sequence for destabilized RNA - 8 nt | GTAAATAG |
| DNA sequence for destabilized RNA - 9 nt | TAAGTTATG |
| DNA sequence for destabilized RNA - 9 nt | TGCTGCCCT |
| DNA sequence for destabilized RNA - 10 nt | TCCTGCCCTC (SEQ ID NO: 59) |
| DNA sequence for destabilized RNA - 11 nt | CCTCCTGCTTA (SEQ ID NO: 60) |
| DNA sequence for destabilized RNA - 11 nt | CCTCGTAACTT (SEQ ID NO: 61) |
| DNA sequence for destabilized RNA - 11 nt | CCTCCTGCCTC (SEQ ID NO: 62) |
| DNA sequence for destabilized RNA - 12 nt | CCTCCTGCATTT (SEQ ID NO: 63) |
| DNA sequence for destabilized RNA - 12 nt | CCTCGCTGCCTC (SEQ ID NO: 64) |
| DNA sequence for destabilized RNA - 14 nt | CTGCTAAGTTAUCT (SEQ ID NO: 65) |
| DNA sequence for destabilized RNA - 16 nt | TAAGTTATCCTCTATT (SEQ ID: 66) |
| DNA sequence for destabilized RNA - 17 nt | TGCCTCTGCTAACTTAT (SEQ ID: 67) |

Exemplary poly A sequences, exemplary promoter sequences, exemplary restriction sequences, and exemplary DNA constructs are shown in the tables below. Nucleotide designation “n”=A, T, G, or C.

TABLE D

| Designation | Sequence/SEQ ID NO: |
|---|---|
| PolyA sequence - 4 nt | AAAA |
| PolyA sequence - 8 nt | AAAAAAAA |
| PolyA sequence - 10 nt | AAAAAAAAAA (SEQ ID NO: 68) |

TABLE E

| Designation | Sequence/SEQ ID NO: |
|---|---|
| MYC1-18 | gagcaatcacctatgaacttgctgctaagttatgatcaaatgcaacctcacaa |
| full length and | ccttggctgagtcttgagactgaaagatttagccataatgtaaactgcctcaa |
| truncations | attggactttgggcataaaagaaccctcctgcatgcttaccatccctcctgcc |
| | ctcctttaacagcctcgtaacttataattgacctcaaaaaacctcaagattta |
| | cacaatgtttctctgtaaatattgccattaaatgtaaataactttaataaaac |
| | gtttatagcagttacacagaatttcaatcctagtatatagtacctagtattat |
| | aggtactataaaccctaacctcctgcatttaagtacattttgcactgcaaagt |
| | aagttatcctctattgacctcagaaaaaataaaataactggcaaatatatcat |
| | tgagccaaatcttaagttgtgaatgctgcctctgctaacttatccctcccaac |
| | caccaccatccctgctgccctcatcaa (SEQ ID NO: 4) |
| | aactcttgtgcgtaaggaaaagtaaggaaaacgattccttctaacagaaa |
| | tgtcctgagcaatcacctatgaacttgtttcaaatgcatgatcaaatgca |
| | acctcacaaccttggctgagtcttgagactgaaagatttagccataatgt |
| | aaactgcctcaaattggactttgggcataaaagaacttttttatgcttac |
| | catcttttttttttctttaacagatttgtatttaagaattgtttttaaaa |
| | aattttaagatttacacaatgtttctctgtaaatattgccattaaatgta |
| | aataactttaataaaacgtttatagcagttacacagaatttcaatcctag |
| | tatatagtacctagtattataggtactataaaccctaattttttttattt |
| | aagtacattttgctttttaaagttgatttttttctattgtttttagaaaa |
| | aataaaataactggcaaatatatcattgagccaaatcttaagttgtgaat |
| | gttttgtttcgtttcttccccctcccaaccaccaccatccctgtttgttt |
| | tcatcaattgccccttcagagggtggtcttaagaaaggcaagagttttcc |
| | tctgttgaaatgggtctgggggccttaaggtctttaagttcttggaggtt |
| | ctaagatgcttcctggagactatgataacagccagagttgacagttagaa |
| | ggaatggcagaaggcaggtgagaaggtgagaggtaggcaaaggagataca |
| | agaggtcaaaggtagcagttaagtacacaaagaggcataaggactgggga |
| | gttgggaggaaggtgaggaagaaactcctgttactttagttaaccagtgc |
| | cagtcccctgctcactccaaacccaggaattctgcccagttgatggggac |
| | acggtgggaaccagcttctgctgccttcacaaccaggcgccagtcctgtc |
| | catgggttatctcgcaaaccccagaggatctctgggaggaatgctactat |
| | taaccctatttcacaaacaaggaaatagaagagctcaaagaggttatgta |
| | acttatctgtagccacgcagataatacaaagcagcaatctggacccattc |
| | tgttcaaaacacttaacccttcgctatcatgccttggttcatctgggtct |
| | aatgtgctgagatcaagaaggtttaggacctaatggacagactcaagtca |
| | taacaatgctaagctctatttgtgtcccaagcactcctaagcattttatc |
| | cctaactctacatcaaccccatgaaggagatactgttgatttccccatat |
| | tagaagtagagagggaagctgaggcacacaaagactcatccacatgccca |
| | agattcactgatagggaaaagtggaagcgagatttgaacccaggctgttt |
| | actcctaacctgtccaagccacctctcagacgacggtaggaatcagctgg |
| | ctgcttgtgagtacaggagttacagtccagtgggttatgttttttaagtc |
| | tcaacatctaagcctggtcaggcatcagttcccctttttttgtgatttat |
| | tttgtttttattttgttgttcattgtttaatttttccttttacaatgaga |
| | aggtcaccatcttgactcctaccttagccatttgttgaatcagactcatg |
| | acggctcctgggaagaagccagttcagatcataaaataaaacatatttat |
| | tctttgtcatgggagtcattattttagaaactacaaactctccttgcttc |
| | catccttttttacatactcatgacacatgctcatcctgagtccttgaaaa |
| | ggtatttttgaacatgtgtattaattataagcctctgaaaacctatggcc |
| | caaaccagaaatgatgttgattatataggtaaatgaaggatgctattgct |
| | gttctaattacctcattgtctcagtctcaaagtaggtcttcagctccctg |
| | tactttgggattttaatctaccaccacccataaatcaataaataattact |
| | ttctttgactctgactcctagaataa (SEQ ID NO: 5) |
| | nnnnnnnnnnttnnnnaganggcacaggnntggtggtggttgggagggataag |
| | ttagcacaggcagcattcacaacttaagatttggctcatgatatatttgccag |
| | ttattttattttttctgaggtcaataaaggataacttactttgtagngcaaaa |
| | tgaacttaaatgcaagaggtaaaggncaatagaacctttaatacaacgtatnn |
| | tttacttngnaaaaaanccaangtnacggcttaaaaccctcnctaaanaanca |
| | ccnntccaaaagtcaattcagacgtacnccncccncnangggccnctaaattn |
| | ntactaccaacntnctgccccctccatacanaaaan (SEQ ID NO: 6) |
| | nnnnnnnnnnttnnnnaganggcacaggnntggtggtggttgggagggataag |
| | ttagcacaggcagcattcacaacttaagatttggctcatgatatatttgccag |
| | ttattttattttttctgaggtcaataaaggataacttactttgtagngcaaaa |
| | tgaacttaaatgcaagaggtaaaggncaatagaacctttaatacaacgtatnn |
| | tttacttngnaaaaaanccaangtnacggcttaaaaccctcnctaaanaanca |
| | CC (SEQ ID NO: 7) |
| | nnnnnnnnnnttnnnnaganggcacaggnntggtggtggttgggagggataag |
| | ttagcacaggcagcattcacaacttaagatttggctcatgatatatttgccag |
| | ttattttattttttctgaggtcaataaaggataacttactttgtagngcaaaa |
| | tgaacttaaatgcaagaggtaaaggncaatagaacctttaatacaacgtatnn |
| | tttacttngnaaaaaanccaangtnacggcttaaaaccctcnctaaanaanca |
| | ccnntccaaaagtcaattcagacgtacnccnccc (SEQ ID NO: 8) |
| | nnnnnnnnnnttnnnnaganggcacaggnntggtggtggttgggagggataag |
| | ttagcacaggcagcattcacaacttaagatttggctcatgatatatttgccag |
| | ttattttattttttctgaggtcaataaaggataacttactttgtagngcaaaa |
| | tgaacttaaatgcaagaggtaaaggncaatagaacctttaatacaacgtatnn |
| | tttacttngnaaaaaanccaangtnacggcttaaaaccctcnctaaanaanca |
| | ccnntccaaaagtcaattcagacgtacnccncccncnangggccnctaaattn |
| | ntactaccaacnt (SEQ ID NO: 9) |
| | NNNNNNNNNNTTNNNNAGANGGCACAGGNNTGGTGGTGGTTGGGAGGGATAAG |
| | TTAGCACAGGCAGCATTCACAACTTAAGATTTGGCTCATGATATATTTGCCAG |
| | TTATTTTATTTTTTCTGAGGTCAATAAAGGATAACTTACTTTGTAGNGCAAAA |
| | TGAACTTAAATGCAAGAGGTAAAGGNCAATAGAACCTTTAATACAACGTATNN |
| | TTTACTTNGNAAAAAANCCAANGTNACGGCTTAAAACCCTCNCTAAANAANCA |
| | CCNNTCCAAAAGTCAATTCAGACGTACNCCNCCCNCNANGGGCCNCTAAATTN |
| | NTACTACCAACNTNCTGCCCCCTCCATACANAAAANCTAAGTGTT (SEQ ID |
| | NO: 10) |
| | NNNNNNNNNNTTNNNNAGANGGCACAGGNNTGGTGGTGGTTGGGAGGGATAAG |
| | TTAGCACAGGCAGCATTCACAACTTAAGATTTGGCTCATGATATATTTGCCAG |
| | TTATTTTATTTTTTCTGAGGTCAATAAAGGATAACTTACTTTGTAGNGCAAAA |
| | TGAACTTAAATGCAAGAGGTAAAGGNCAATAGAACCTTTAATACAACGTATNN |
| | TTTACTTNGNAAAAAANCCAANGTNACGGCTTAAAACCCTCNCTAAANAANCA |
| | CCNNTCCAAAAGTCAATTCAGACGTACNCCNCCCNCNANGGGCCNCTAAATTN |
| | NTACTACCAACNTNCTGCCCCCTCCATACANAAAANCTAAGTGTTGGCTTTAA |
| | AGTCCTAGGGGCCGGCCCCGCCG (SEQ ID NO: 11) |
| ERBB2-30 | caggggaacctgccatgccaggaacctgtcctaaggaacctgccttcctgctt |
| full length and | gagttcccagatggctggaaggggtccagcctcgttggaagaggaacagcact |
| truncations | ggggagtcttcgtggattctgaggccctgcccaatgagactctagggtccagt |
| | ggatgccacagcccagcttggccctctccttccagatcctgggtactgaaagc |
| | cttagggaagctggcctgagaggggaagcggccctaagggagtgtctaagaac |
| | aaaagcgacccattcagagactgtccctgaaacctagtactgccccccatgag |
| | gaaggaacagcaatggtgtcagtatccaggtcgcgttcagagtgccctcctgc |
| | ttagctgctaagttatctgcctcgctgcctcaaagatgaaataaagacccagg |
| | gggagaatgggtgttgtatggggaggcaagtgtggggggtccttctccacacc |
| | cactttgtccatttgcaaatatat (SEQ ID NO: 12) |
| | agaaggccaagtccgcagaagccctgatgtgtcctcagggagcagggaag |
| | gcctgacttctgctggcatcaagaggtgggagggccctccgaccacttcc |
| | aggggaacctgccatgccaggaacctgtcctaaggaaccttccttcctgc |
| | ttgagttcccagatggctggaaggggtccagcctcgttggaagaggaaca |
| | gcactggggagtctttgtggattctgaggccctgcccaatgagactctag |
| | ggtccagtggatgccacagcccagcttggccctttccttccagatcctgg |
| | gtactgaaagccttagggaagctggcctgagaggggaagcggccctaagg |
| | gagtgtctaagaacaaaagcgacccattcagagactgtccctgaaaccta |
| | gtactgccccccatgaggaaggaacagcaatggtgtcagtatccaggctt |
| | tgtacagagtgcttttctgtttagtttttactttttttgttttgtttttt |
| | taaagatgaaataaagacccagggggagaatgggtgttgtatggggaggc |
| | aagtgtggggggtccttctccacacccactttgtccatttgcaaatatat |
| | tttggaaaacagctaggc (SEQ ID NO: 13) |
| | nnnnnnntnnnnnnnnnnnnngnnnccntangtttctttttatatatttcgct |
| | ctggaactgcgggcgtggagaaggaccccccacactggcctccccatacaaca |
| | cccattctccccctgggtctttatttcgtctttgaggccgcgggggacatgac |
| | ttaccacctaaccatgagggcactctcaacgcgacctggatactgacaccatt |
| | gctgttccttcctcatggggggcagtactaagtttcagggacagtctctgaat |
| | gggtcgcttttgttcttangtctcccttatggcctcttcccctctcaagccaa |
| | tttccntaaggatttctctaccaangatctggaaaaaaagggccaatttgggc |
| | tgtggcatccccaggaccnnacattgcatgaaagctaatgccgacaaggatca |
| | ataaaaaactcttanattatttcttaatacttcccctacacttn (SEQ ID |
| | NO: 14) |
| | nnnnnnntnnnnnnnnnnnnngnnnccntangtttctttttatatatttcgct |
| | ctggaactgcgggcgtggagaaggaccccccacactggcctccccatacaaca |
| | cccattctccccctgggtctttatttcgtctttgaggccgcgggggacatgac |
| | ttaccacctaaccatgagggcactctcaacgcgacctggatactgacaccatt |
| | gctgttccttcctcatggggggcagtactaagtttcagggacagtctctgaat |
| | gggtcgcttttgttcttangtctcccttatggcctcttcccctctcaagccaa |
| | tttccntaaggatttctctaccaangatctggaaaaaaagg (SEQ ID NO: |
| | 15) |
| | nnnnnnntnnnnnnnnnnnnngnnnccntangtttctttttatatatttcgct |
| | ctggaactgcgggcgtggagaaggaccccccacactggcctccccatacaaca |
| | cccattctccccctgggtctttatttcgtctttgaggccgcgggggacatgac |
| | ttaccacctaaccatgagggcactctcaacgcgacctggatactgacaccatt |
| | gctgttccttcctcatggggggcagtactaagtttcagggacagtctctgaat |
| | gggtcgcttttgttcttangtctcccttatggcctcttcccctctcaagccaa |
| | tttccntaaggatttctctaccaangatctggaaaaaaagggccaatttgggc |
| | tgtggcatccccaggaccnnacattg (SEQ ID NO: 16) |
| | nnnnnnntnnnnnnnnnnnnngnnnccntangtttctttttatatatttcgct |
| | ctggaactgcgggcgtggagaaggaccccccacactggcctccccatacaaca |
| | cccattctccccctgggtctttatttcgtctttgaggccgcgggggacatgac |
| | ttaccacctaaccatgagggcactctcaacgcgacctggatactgacaccatt |
| | gctgttccttcctcatggggggcagtactaagtttcagggacagtctctgaat |
| | gggtcgcttttgttcttangtctcccttatggcctcttcccctctcaagccaa |
| | tttccntaaggatttctctaccaangatctggaaaaaaagggccaatttgggc |
| | tgtggcatccccaggaccnnacattgcatgaaagctaatgccgacaaggatca |
| | ataaaaaact (SEQ ID NO: 17) |
| | nnnnnnntnnnnnnnnnnnnngnnnccntangtttctttttatatatttcgct |
| | ctggaactgcgggcgtggagaaggaccccccacactggcctccccatacaaca |
| | cccattctccccctgggtctttatttcgtctttgaggccgcgggggacatgac |
| | ttaccacctaaccatgagggcactctcaacgcgacctggatactgacaccatt |
| | gctgttccttcctcatggggggcagtactaagtttcagggacagtctctgaat |
| | gggtcgcttttgttcttangtctcccttatggcctcttcccctctcaagccaa |
| | tttccntaaggatttctctaccaangatctggaaaaaaagggccaatttgggc |
| | tgtggcatccccaggaccnnacattgcatgaaagctaatgccgacaaggatca |
| | ataaaaaactcttanattatttcttaatacttcccctacacttncta (SEQ |
| | ID NO: 18) |
| | nnnnnnntnnnnnnnnnnnnngnnnccntangtttctttttatatatttcgct |
| | ctggaactgcgggcgtggagaaggaccccccacactggcctccccatacaaca |
| | cccattctccccctgggtctttatttcgtctttgaggccgcgggggacatgac |
| | ttaccacctaaccatgagggcactctcaacgcgacctggatactgacaccatt |
| | gctgttccttcctcatggggggcagtactaagtttcagggacagtctctgaat |
| | gggtcgcttttgttcttangtctcccttatggcctcttcccctctcaagccaa |
| | tttccntaaggatttctctaccaangatctggaaaaaaagggccaatttgggc |
| | tgtggcatccccaggaccnnacattgcatgaaagctaatgccgacaaggatca |
| | ataaaaaactcttanattatttcttaatacttcccctacacttnctaagtgtt |
| | ggctttaaagtcctaggggccggccccgccg (SEQ ID NO: 19) |
| MYCN1-18 | nnnnnnnannnnnntctcnnncttcagcctcnatacgcgtgtcccggcnggac |
| | ggtacttcctccnttctggtcctgcccccaggcccgtatagaagcttaaatgc |
| | aggagatgaacaatcatgggggaattttgcctcgatggcattgcgttgcanac |
| | ctcctgcctccaaacaaacattgtgttgacattaagaatgttggtttactttc |
| | aaatcggtcccctgtcgagttcggctctgggtgggcagtaggaccaccagtgt |
| | ggggttctgctgggaccttggagagcctgcatcccaggatgctgggtggccct |
| | gcagcctcctccacctcacctccatgacagcgctaaacgttggtgacggttgg |
| | gagcctctggggctgttgaagtcaccttgtgtgttccaagtttccaaacaaca |
| | gaaagtcattccttcctcttaaaatggtgcttaagttccagcagatgccacat |
| | aaggggtttgccatttgatacccctggggaacatttctgtaaataccattgac |
| | acatccgccttttgtatacatcctgggtaatgagaggtggcttttgcggccag |
| | tattagactggagttcatacctaagtactgtaataatacctcaatgtttgagg |
| | agcatgcctcgtatacaaatatattgttaatctctgttatgtactgtactaat |
| | tcttacactgcctgtatactttagtatgacgctgatacataactaaatttgat |
| | acttatattttcgtatgaaaatgagttgtgaaagttttgagtagatattacct |
| | catcacctcttgaactaagaaacttttgtaaagaaatttactatatatatatg |
| | cctctgc (SEQ ID NO: 20) |
| MYCN1-14 | cctcctgcctccaaacaaacattgtgttgacattaagaatgttggtttacttt |
| full length and | caaatcggtcccctgtcgagttcggctctgggtgggcagtaggaccaccagtg |
| truncations | tggggttctgctgggaccttggagagcctgcatcccaggatgctgggtggccc |
| | tgcagcctcctccacctcacctccatgacagcgctaaacgttggtgacggttg |
| | ggagcctctggggctgttgaagtcaccttgtgtgttccaagtttccaaacaac |
| | agaaagtcattccttcctcttaaaatggtgcttaagttccagcagatgccaca |
| | taaggggtttgccatttgatacccctggggaacatttctgtaaataccattga |
| | cacatccgccttttgtatacatcctgggtaatgagaggtggcttttgcggcca |
| | gtattagactggagttcatacctaagtactgtaataatacctcaatgtttgag |
| | gagcatgcctcgtatacaaatatattgttaatctctgttatgtactgtactaa |
| | ttcttacactgcctgtatactttagtatgacgctgatacataactaaatttga |
| | tacttatattttcgtatgaaaatgagttgtgaaagttttgagtagatattacc |
| | tcatcacctcttgaactaagaaacttttgtaaagaaatttactatatatatat |
| | gcctctgc (SEQ ID NO: 21) |
| | nnnnnnnnnnnnnntcnctcancttcngcctctacacgggcctcgcggcaggg |
| | cggtacttcagccattctggtcctgcccgcagatgcgtatnnnngcttgaatg |
| | caggagctgaacaatcacctatgaattttgcctcgatggcattgcgttgannn |
| | cctcctgcctccaaacaaacattgtgttgacattaagaatgttggtttacttt |
| | caaatcggtcccctgtcgagttcggctctgggtgggcagtaggaccaccagtg |
| | tggggttctgctgggaccttggagagcctgcatcccaggatgctgggtggccc |
| | tgcagcctcctccacctcacctccatgacagcgctaaacgttggtgacggttg |
| | ggagcctctggggctgttgaagtcaccttgtgtgttccaagtttccaaacaac |
| | agaaagtcattccttcctcttaaaatggtgcttaagttccagcagatgccaca |
| | taaggggtttgccatttgatacccctggggaacatttctgtaaataccattga |
| | cacatccgccttttgtatacatcctgggtaatgagaggtggcttttgcggcca |
| | gtattagactggagttcatacctaagtactgtaataatacctcaatgtttgag |
| | gagcatgcctcgtatacaaatatattgttaatctctgttatgtactgtactaa |
| | ttcttacactgcctgtatactttagtatgacgctgatacataactaaatttga |
| | tacttatattttcgtatgaaaatgagttgtgaaagttttgagtagatattacc |
| | tcatcacctcttgaactaagaaacttttgtaaagaaatttactatatatatat |
| | gcctctgc (SEQ ID NO: 22) |
| | nnnnnnnnnnnnnntcnctcancttcngcctctacacgggcctcgcggcaggg |
| | cggtacttcagccattctggtcctgcccgcagatgcgtatnnnngcttgaatg |
| | caggagctgaacaatcacctatgaattttgcctcgatggcattgcgttgannn |
| | cctcctgcctccaaacaaacattgtgttgacattaagaatgttggtttacttt |
| | caaatcggtcccctgtcgagttcggctctgggtgggcagtaggaccaccagtg |
| | tggggttctgctgggaccttggagagcctgcatcccaggatgctgggtggccc |
| | tgcagcctcctccacctcacctccatgacagcgctaaacgttggtgacggttg |
| | ggagcctctggggctgttgaagtcaccttgtgtgttccaagtttccaaacaac |
| | agaaagtcattccttcctcttaaaatggtgcttaagttccagcagatgccaca |
| | taaggggtttgccatttgatacccctggggaacatttctgtaaataccattga |
| | cacatccgccttttgtatacatcctgggtaatgagaggtggcttttgcggcca |
| | gtattagactggagttcatacctaagtactgtaataatacctcaatgtttgag |
| | gagcatgcctcgtatacaaatatattgttaatctctgttatgtactgta |
| | (SEQ ID NO: 23) |
| | nnnnnnnnnnnnnntcnctcancttcngcctctacacgggcctcgcggcaggg |
| | cggtacttcagccattctggtcctgcccgcagatgcgtatnnnngcttgaatg |
| | caggagctgaacaatcacctatgaattttgcctcgatggcattgcgttgannn |
| | cctcctgcctccaaacaaacattgtgttgacattaagaatgttggtttacttt |
| | caaatcggtcccctgtcgagttcggctctgggtgggcagtaggaccaccagtg |
| | tggggttctgctgggaccttggagagcctgcatcccaggatgctgggtggccc |
| | tgcagcctcctccacctcacctccatgacagcgctaaacgttggtgacggttg |
| | ggagcctctggggctgttgaagtcaccttgtgtgttccaagtttccaaacaac |
| | agaaagtcattccttcctcttaaaatggtgcttaagttccagcagatgccaca |
| | taaggggtttgccatttgatacccctggggaacatttctgtaaataccattga |
| | cacatccgccttttgtatacatcctgggtaatgagaggtggcttttgcggcca |
| | gtattagactggagttcatacctaagtactgtaataatacctcaatgtttgag |
| | gagcatgcctcgtatacaaatatattgttaatctctgttatgtactgtactaa |
| | ttcttacactgcctgtatactttagtatgacgctgata (SEQ ID NO: 24) |
| | nnnnnnnnnnnnnntcnctcancttcngcctctacacgggcctcgcggcaggg |
| | cggtacttcagccattctggtcctgcccgcagatgcgtatnnnngcttgaatg |
| | caggagctgaacaatcacctatgaattttgcctcgatggcattgcgttgannn |
| | cctcctgcctccaaacaaacattgtgttgacattaagaatgttggtttacttt |
| | caaatcggtcccctgtcgagttcggctctgggtgggcagtaggaccaccagtg |
| | tggggttctgctgggaccttggagagcctgcatcccaggatgctgggtggccc |
| | tgcagcctcctccacctcacctccatgacagcgctaaacgttggtgacggttg |
| | ggagcctctggggctgttgaagtcaccttgtgtgttccaagtttccaaacaac |
| | agaaagtcattccttcctcttaaaatggtgcttaagttccagcagatgccaca |
| | taaggggtttgccatttgatacccctggggaacatttctgtaaataccattga |
| | cacatccgccttttgtatacatcctgggtaatgagaggtggcttttgcggcca |
| | gtattagactggagttcatacctaagtactgtaataatacctcaatgtttgag |
| | gagcatgcctcgtatacaaatatattgttaatctctgttatgtactgtactaa |
| | ttcttacactgcctgtatactttagtatgacgctgatacataactaaatttga |
| | tacttatattttcgtatgaaaatgagtt (SEQ ID NO: 25) |
| | nnnnnnnnnnnnnntcnctcancttcngcctctacacgggcctcgcggcaggg |
| | cggtacttcagccattctggtcctgcccgcagatgcgtatnnnngcttgaatg |
| | caggagctgaacaatcacctatgaattttgcctcgatggcattgcgttgannn |
| | cctcctgcctccaaacaaacattgtgttgacattaagaatgttggtttacttt |
| | caaatcggtcccctgtcgagttcggctctgggtgggcagtaggaccaccagtg |
| | tggggttctgctgggaccttggagagcctgcatcccaggatgctgggtggccc |
| | tgcagcctcctccacctcacctccatgacagcgctaaacgttggtgacggttg |
| | ggagcctctggggctgttgaagtcaccttgtgtgttccaagtttccaaacaac |
| | agaaagtcattccttcctcttaaaatggtgcttaagttccagcagatgccaca |
| | taaggggtttgccatttgatacccctggggaacatttctgtaaataccattga |
| | cacatccgccttttgtatacatcctgggtaatgagaggtggcttttgcggcca |
| | gtattagactggagttcatacctaagtactgtaataatacctcaatgtttgag |
| | gagcatgcctcgtatacaaatatattgttaatctctgttatgtactgtactaa |
| | ttcttacactgcctgtatactttagtatgacgctgatacataactaaatttga |
| | tacttatattttcgtatgaaaatgagttgtgaaagttttgagtagatattacc |
| | tcatcacctcttgaacta (SEQ ID NO: 26) |
| | nnnnnnnnnnnnnntcnctcancttcngcctctacacgggcctcgcggcaggg |
| | cggtacttcagccattctggtcctgcccgcagatgcgtatnnnngcttgaatg |
| | caggagctgaacaatcacctatgaattttgcctcgatggcattgcgttgannn |
| | cctcctgcctccaaacaaacattgtgttgacattaagaatgttggtttacttt |
| | caaatcggtcccctgtcgagttcggctctgggtgggcagtaggaccaccagtg |
| | tggggttctgctgggaccttggagagcctgcatcccaggatgctgggtggccc |
| | tgcagcctcctccacctcacctccatgacagcgctaaacgttggtgacggttg |
| | ggagcctctggggctgttgaagtcaccttgtgtgttccaagtttccaaacaac |
| | agaaagtcattccttcctcttaaaatggtgcttaagttccagcagatgccaca |
| | taaggggtttgccatttgatacccctggggaacatttctgtaaataccattga |
| | cacatccgccttttgtatacatcctgggtaatgagaggtggcttttgcggcca |
| | gtattagactggagttcatacctaagtactgtaataatacctcaatgtttgag |
| | gagcatgcctcgtatacaaatatattgttaatctctgttatgtactgtactaa |
| | ttcttacactgcctgtatactttagtatgacgctgatacataactaaatttga |
| | tacttatattttcgtatgaaaatgagttgtgaaagttttgagtagatattacc |
| | tcatcacctcttgaacta (SEQ ID NO: 27) |

TABLE F

| | |
|---|---|
| DCPIA | gccatcgaggcaaaattcccccatctgcgtcagtcccctcaacttccgcctc |
| promoter | tacgcgggcctcgcggcagggcggtacgtcagccattctggtccgccgcgcg |
| | cacgctccgggcgccgggttccggcgttgtcagggtccgcggccctacgatg |
| | tgggcggtgtccaaggctgcgtagtggagcttgcaggctggagct (SEQ ID |
| | NO: 28) |
| DCPIA | gccatcgaggcaaaattcccccatctgcgtcagtcccctcaacttccgcctc |
| promoter- | tacgcgggcctcgcggcagggcggtacgtcagccattctggtccgccgcgcg |
| shortened | catgcgtagtggagcttgcaggctggagct (SEQ ID NO: 29) |
| MYC1-18 | GCCCGCGAGGACCCGCCCGAGCTTCGAAAAAAAAAAGCCATCGAGGCAAAAT |
| sequences with | TCCCCCATCTGCGTCAGTCCCCTCAACTTCCGCCTCTACGCGGGCCTCGCGG |
| promoter | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTgagcaatcacctatgaacttgct |
| | gctaagttatgatcaaatgcaacctcacaaccttggctgagtcttgagactg |
| | aaagatttagccataatgtaaactgcctcaaattggactttgggcataaaag |
| | aaccctcctgcatgcttaccatccctcctgccctcctttaacagcctcgtaa |
| | cttataattgacctcaaaaaacctcaagatttacacaatgtttctctgtaaa |
| | tattgccattaaatgtaaataactttaataaaacgtttatagcagttacaca |
| | gaatttcaatcctagtatatagtacctagtattataggtactataaacccta |
| | acctcctgcatttaagtacattttgcactgcaaagtaagttatcctctattg |
| | acctcagaaaaaataaaataactggcaaatatatcattgagccaaatcttaa |
| | gttgtgaatgctgcctctgctaacttatccctcccaaccaccaccatccctg |
| | ctgccctcatcaa (SEQ ID NO: 30) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTnnnnnnnnnnttnnnnaganggc |
| | acaggnntggtggtggttgggagggataagttagcacaggcagcattcacaa |
| | cttaagatttggctcatgatatatttgccagttattttattttttctgaggt |
| | caataaaggataacttactttgtagngcaaaatgaacttaaatgcaagaggt |
| | aaaggncaatagaacctttaatacaacgtatnntttacttngnaaaaaancc |
| | aangtnacggcttaaaaccctcnctaaanaancacc (SEQ ID NO: 31) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTnnnnnnnnnnttnnnnaganggc |
| | acaggnntggtggtggttgggagggataagttagcacaggcagcattcacaa |
| | cttaagatttggctcatgatatatttgccagttattttattttttctgaggt |
| | caataaaggataacttactttgtagngcaaaatgaacttaaatgcaagaggt |
| | aaaggncaatagaacctttaatacaacgtatnntttacttngnaaaaaancc |
| | aangtnacggcttaaaaccctcnctaaanaancaccnntccaaaagtcaatt |
| | cagacgtacnccnccc (SEQ ID NO: 32) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTnnnnnnnnnnttnnnnaganggc |
| | acaggnntggtggtggttgggagggataagttagcacaggcagcattcacaa |
| | cttaagatttggctcatgatatatttgccagttattttattttttctgaggt |
| | caataaaggataacttactttgtagngcaaaatgaacttaaatgcaagaggt |
| | aaaggncaatagaacctttaatacaacgtatnntttacttngnaaaaaancc |
| | aangtnacggcttaaaaccctcnctaaanaancaccnntccaaaagtcaatt |
| | cagacgtacnccncccncnangggccnctaaattnntactaccaacnt |
| | (SEQ ID NO: 33) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTnnnnnnnnnnttnnnnaganggc |
| | acaggnntggtggtggttgggagggataagttagcacaggcagcattcacaa |
| | cttaagatttggctcatgatatatttgccagttattttattttttctgaggt |
| | caataaaggataacttactttgtagngcaaaatgaacttaaatgcaagaggt |
| | aaaggncaatagaacctttaatacaacgtatnntttacttngnaaaaaancc |
| | aangtnacggcttaaaaccctcnctaaanaancaccnntccaaaagtcaatt |
| | cagacgtacnccncccncnangggccnctaaattnntactaccaacntnctg |
| | ccccctccatacanaaaanctaagtgtt (SEQ ID NO: 34) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTnnnnnnnnnnttnnnnaganggc |
| | acaggnntggtggtggttgggagggataagttagcacaggcagcattcacaa |
| | cttaagatttggctcatgatatatttgccagttattttattttttctgaggt |
| | caataaaggataacttactttgtagngcaaaatgaacttaaatgcaagaggt |
| | aaaggncaatagaacctttaatacaacgtatnntttacttngnaaaaaancc |
| | aangtnacggcttaaaaccctcnctaaanaancaccnntccaaaagtcaatt |
| | cagacgtacnccncccncnangggccnctaaattnntactaccaacntnctg |
| | ccccctccatacanaaaanctaagtgttggctttaaagtcctaggggccggc |
| | cccgccg (SEQ ID NO: 35) |
| ERBB2-30 | GCCCGCGAGGACCCGCCCGAGCTTCGAAAAAAAAAAGCCATCGAGGCAAAAT |
| sequences with | TCCCCCATCTGCGTCAGTCCCCTCAACTTCCGCCTCTACGCGGGCCTCGCGG |
| promoter | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTcaggggaacctgccatgccagga |
| | acctgtcctaaggaacctgccttcctgcttgagttcccagatggctggaagg |
| | ggtccagcctcgttggaagaggaacagcactggggagtcttcgtggattctg |
| | aggccctgcccaatgagactctagggtccagtggatgccacagcccagcttg |
| | gccctctccttccagatcctgggtactgaaagccttagggaagctggcctga |
| | gaggggaagcggccctaagggagtgtctaagaacaaaagcgacccattcaga |
| | gactgtccctgaaacctagtactgccccccatgaggaaggaacagcaatggt |
| | gtcagtatccaggtcgcgttcagagtgccctcctgcttagctgctaagttat |
| | ctgcctcgctgcctcaaagatgaaataaagacccagggggagaatgggtgtt |
| | gtatggggaggcaagtgtggggggtccttctccacacccactttgtccattt |
| | gcaaatatat (SEQ ID NO: 36) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTNNNNNNNTNNNNNNNNNNNNNGN |
| | NNCCNTANGTTTCTTTTTATATATTTCGCTCTGGAACTGCGGGCGTGGAGAA |
| | GGACCCCCCACACTGGCCTCCCCATACAACACCCATTCTCCCCCTGGGTCTT |
| | TATTTCGTCTTTGAGGCCGCGGGGGACATGACTTACCACCTAACCATGAGGG |
| | CACTCTCAACGCGACCTGGATACTGACACCATTGCTGTTCCTTCCTCATGGG |
| | GGGCAGTACTAAGTTTCAGGGACAGTCTCTGAATGGGTCGCTTTTGTTCTTA |
| | NGTCTCCCTTATGGCCTCTTCCCCTCTCAAGCCAATTTCCNTAAGGATTTCT |
| | CTACCAANGATCTGGAAAAAAAGGGCCAATTTGGGCTGTGGCATCCCCAGGA |
| | CCNNACATTGCATGAAAGCTAATGCCGACAAGGATCAATAAAAAACTCTTAN |
| | ATTATTTCTTAATACTTCCCCTACACTTNCTAAGTGTTGGCTTTAAAGTCCT |
| | AGGGGCCGGCCCCGCCG (SEQ ID NO: 37) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTNNNNNNNTNNNNNNNNNNNNNGN |
| | NNCCNTANGTTTCTTTTTATATATTTCGCTCTGGAACTGCGGGCGTGGAGAA |
| | GGACCCCCCACACTGGCCTCCCCATACAACACCCATTCTCCCCCTGGGTCTT |
| | TATTTCGTCTTTGAGGCCGCGGGGGACATGACTTACCACCTAACCATGAGGG |
| | CACTCTCAACGCGACCTGGATACTGACACCATTGCTGTTCCTTCCTCATGGG |
| | GGGCAGTACTAAGTTTCAGGGACAGTCTCTGAATGGGTCGCTTTTGTTCTTA |
| | NGTCTCCCTTATGGCCTCTTCCCCTCTCAAGCCAATTTCCNTAAGGATTTCT |
| | CTACCAANGATCTGGAAAAAAAGG (SEQ ID NO: 38) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTnnnnnnntnnnnnnnnnnnnngn |
| | nnccntangtttctttttatatatttcgctctggaactgcgggcgtggagaa |
| | ggaccccccacactggcctccccatacaacacccattctccccctgggtctt |
| | tatttcgtctttgaggccgcgggggacatgacttaccacctaaccatgaggg |
| | cactctcaacgcgacctggatactgacaccattgctgttccttcctcatggg |
| | gggcagtactaagtttcagggacagtctctgaatgggtcgcttttgttctta |
| | ngtctcccttatggcctcttcccctctcaagccaatttccntaaggatttct |
| | ctaccaangatctggaaaaaaagggccaatttgggctgtggcatccccagga |
| | ccnnacattg (SEQ ID NO: 39) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTNNNNNNNTNNNNNNNNNNNNNGN |
| | NNCCNTANGTTTCTTTTTATATATTTCGCTCTGGAACTGCGGGCGTGGAGAA |
| | GGACCCCCCACACTGGCCTCCCCATACAACACCCATTCTCCCCCTGGGTCTT |
| | TATTTCGTCTTTGAGGCCGCGGGGGACATGACTTACCACCTAACCATGAGGG |
| | CACTCTCAACGCGACCTGGATACTGACACCATTGCTGTTCCTTCCTCATGGG |
| | GGGCAGTACTAAGTTTCAGGGACAGTCTCTGAATGGGTCGCTTTTGTTCTTA |
| | NGTCTCCCTTATGGCCTCTTCCCCTCTCAAGCCAATTTCCNTAAGGATTTCT |
| | CTACCAANGATCTGGAAAAAAAGGGCCAATTTGGGCTGTGGCATCCCCAGGA |
| | CCNNACATTGCATGAAAGCTAATGCCGACAAGGATCAATAAAAAACT (SEQ |
| | ID NO: 40) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTnnnnnnntnnnnnnnnnnnnngn |
| | nnccntangtttctttttatatatttcgctctggaactgcgggcgtggagaa |
| | ggaccccccacactggcctccccatacaacacccattctccccctgggtctt |
| | tatttcgtctttgaggccgcgggggacatgacttaccacctaaccatgaggg |
| | cactctcaacgcgacctggatactgacaccattgctgttccttcctcatggg |
| | gggcagtactaagtttcagggacagtctctgaatgggtcgcttttgttctta |
| | ngtctcccttatggcctcttcccctctcaagccaatttccntaaggatttct |
| | ctaccaangatctggaaaaaaagggccaatttgggctgtggcatccccagga |
| | ccnnacattgcatgaaagctaatgccgacaaggatcaataaaaaactcttan |
| | attatttcttaatacttcccctacacttncta (SEQ ID NO: 41) |
| | GCCCGCGAGGACCCGCCCGAGCTTCGAAAAAAAAAAGCCATCGAGGCAAAAT |
| | TCCCCCATCTGCGTCAGTCCCCTCAACTTCCGCCTCTACGCGGGCCTCGCGG |
| | CAGGGCGGTACGTCAGCCATTCTGGTCCGCCGCGCGCACGCTCCGGGCGCCG |
| | GGTTCCGGCGTTGTCAGGGTCCGCGGCCCTACGATGTGGGCGGTGTCCAAGG |
| | CTGCGTAGTGGAGCTTGCAGGCTGGAGCTnnnnnnntnnnnnnnnnnnnngn |
| | nnccntangtttctttttatatatttcgctctggaactgcgggcgtggagaa |
| | ggaccccccacactggcctccccatacaacacccattctccccctgggtctt |
| | tatttcgtctttgaggccgcgggggacatgacttaccacctaaccatgaggg |
| | cactctcaacgcgacctggatactgacaccattgctgttccttcctcatggg |
| | gggcagtactaagtttcagggacagtctctgaatgggtcgcttttgttctta |
| | ngtctcccttatggcctcttcccctctcaagccaatttccntaaggatttct |
| | ctaccaangatctggaaaaaaagggccaatttgggctgtggcatccccagga |
| | ccnnacattgcatgaaagctaatgccgacaaggatcaataaaaaactcttan |
| | attatttcttaatacttcccctacacttnctaagtgttggctttaaagtcct |
| | aggggccggccccgccg |
| | (SEQ ID NO: 42) |
| MYCN1-14 | GCCATCGAGGCAAAATTCCCCCATCTGCGTCAGTCCCCTCAACTTCCGCCTC |
| sequences with | TACGCGGGCCTCGCGGCAGGGCGGTACGTCAGCCATTCTGGTCCGCCGCGCG |
| promoter | CATGCGTAGTGGAGCTTGCAGGCTGGAGCTcctcctgcctccaaacaaacat |
| | tgtgttgacattaagaatgttggtttactttcaaatcggtcccctgtcgagt |
| | tcggctctgggtgggcagtaggaccaccagtgtggggttctgctgggacctt |
| | ggagagcctgcatcccaggatgctgggtggccctgcagcctcctccacctca |
| | cctccatgacagcgctaaacgttggtgacggttgggagcctctggggctgtt |
| | gaagtcaccttgtgtgttccaagtttccaaacaacagaaagtcattccttcc |
| | tcttaaaatggtgcttaagttccagcagatgccacataaggggtttgccatt |
| | tgatacccctggggaacatttctgtaaataccattgacacatccgccttttg |
| | tatacatcctgggtaatgagaggtggcttttgcggccagtattagactggag |
| | ttcatacctaagtactgtaataatacctcaatgtttgaggagcatgcctcgt |
| | atacaaatatattgttaatctctgttatgtactgtactaattcttacactgc |
| | ctgtatactttagtatgacgctgatacataactaaatttgatacttatattt |
| | tcgtatgaaaatgagttgtgaaagttttgagtagatattacctcatcacctc |
| | ttgaactaagaaacttttgtaaagaaatttactatatatatatgcctctgc |
| | (SEQ ID NO: 43) |
| | GCCATCGAGGCAAAATTCCCCCATCTGCGTCAGTCCCCTCAACTTCCGCCTC |
| | TACGCGGGCCTCGCGGCAGGGCGGTACGTCAGCCATTCTGGTCCGCCGCGCG |
| | CATGCGTAGTGGAGCTTGCAGGCTGGAGCTnnnnnnnnnnnnnntcnctcan |
| | cttcngcctctacacgggcctcgcggcagggcggtacttcagccattctggt |
| | cctgcccgcagatgcgtatnnnngcttgaatgcaggagctgaacaatcacct |
| | atgaattttgcctcgatggcattgcgttgannncctcctgcctccaaacaaa |
| | cattgtgttgacattaagaatgttggtttactttcaaatcggtcccctgtcg |
| | agttcggctctgggtgggcagtaggaccaccagtgtggggttctgctgggac |
| | cttggagagcctgcatcccaggatgctgggtggccctgcagcctcctccacc |
| | tcacctccatgacagcgctaaacgttggtgacggttgggagcctctggggct |
| | gttgaagtcaccttgtgtgttccaagtttccaaacaacagaaagtcattcct |
| | tcctcttaaaatggtgcttaagttccagcagatgccacataaggggtttgcc |
| | atttgatacccctggggaacatttctgtaaataccattgacacatccgcctt |
| | ttgtatacatcctgggtaatgagaggtggcttttgcggccagtattagactg |
| | gagttcatacctaagtactgtaataatacctcaatgtttgaggagcatgcct |
| | cgtatacaaatatattgttaatctctgttatgtactgta (SEQ ID NO: 44) |
| | GCCATCGAGGCAAAATTCCCCCATCTGCGTCAGTCCCCTCAACTTCCGCCTC |
| | TACGCGGGCCTCGCGGCAGGGCGGTACGTCAGCCATTCTGGTCCGCCGCGCG |
| | CATGCGTAGTGGAGCTTGCAGGCTGGAGCTnnnnnnnnnnnnnntcnctcan |
| | cttcngcctctacacgggcctcgcggcagggcggtacttcagccattctggt |
| | cctgcccgcagatgcgtatnnnngcttgaatgcaggagctgaacaatcacct |
| | atgaattttgcctcgatggcattgcgttgannncctcctgcctccaaacaaa |
| | cattgtgttgacattaagaatgttggtttactttcaaatcggtcccctgtcg |
| | agttcggctctgggtgggcagtaggaccaccagtgtggggttctgctgggac |
| | cttggagagcctgcatcccaggatgctgggtggccctgcagcctcctccacc |
| | tcacctccatgacagcgctaaacgttggtgacggttgggagcctctggggct |
| | gttgaagtcaccttgtgtgttccaagtttccaaacaacagaaagtcattcct |
| | tcctcttaaaatggtgcttaagttccagcagatgccacataaggggtttgcc |
| | atttgatacccctggggaacatttctgtaaataccattgacacatccgcctt |
| | ttgtatacatcctgggtaatgagaggtggcttttgcggccagtattagactg |
| | gagttcatacctaagtactgtaataatacctcaatgtttgaggagcatgcct |
| | cgtatacaaatatattgttaatctctgttatgtactgtactaattcttacac |
| | tgcctgtatactttagtatgacgctgata(SEQ ID NO: 45) |
| | GCCATCGAGGCAAAATTCCCCCATCTGCGTCAGTCCCCTCAACTTCCGCCTC |
| | TACGCGGGCCTCGCGGCAGGGCGGTACGTCAGCCATTCTGGTCCGCCGCGCG |
| | CATGCGTAGTGGAGCTTGCAGGCTGGAGCTnnnnnnnnnnnnnntcnctcan |
| | cttcngcctctacacgggcctcgcggcagggcggtacttcagccattctggt |
| | cctgcccgcagatgcgtatnnnngcttgaatgcaggagctgaacaatcacct |
| | atgaattttgcctcgatggcattgcgttgannncctcctgcctccaaacaaa |
| | cattgtgttgacattaagaatgttggtttactttcaaatcggtcccctgtcg |
| | agttcggctctgggtgggcagtaggaccaccagtgtggggttctgctgggac |
| | cttggagagcctgcatcccaggatgctgggtggccctgcagcctcctccacc |
| | tcacctccatgacagcgctaaacgttggtgacggttgggagcctctggggct |
| | gttgaagtcaccttgtgtgttccaagtttccaaacaacagaaagtcattcct |
| | tcctcttaaaatggtgcttaagttccagcagatgccacataaggggtttgcc |
| | atttgatacccctggggaacatttctgtaaataccattgacacatccgcctt |
| | ttgtatacatcctgggtaatgagaggtggcttttgcggccagtattagactg |
| | gagttcatacctaagtactgtaataatacctcaatgtttgaggagcatgcct |
| | cgtatacaaatatattgttaatctctgttatgtactgtactaattcttacac |
| | tgcctgtatactttagtatgacgctgatacataactaaatttgatacttata |
| | ttttcgtatgaaaatgagtt (SEQ ID NO: 46) |
| | GCCATCGAGGCAAAATTCCCCCATCTGCGTCAGTCCCCTCAACTTCCGCCTC |
| | TACGCGGGCCTCGCGGCAGGGCGGTACGTCAGCCATTCTGGTCCGCCGCGCG |
| | CATGCGTAGTGGAGCTTGCAGGCTGGAGCTnnnnnnnnnnnnnntcnctcan |
| | cttcngcctctacacgggcctcgcggcagggcggtacttcagccattctggt |
| | cctgcccgcagatgcgtatnnnngcttgaatgcaggagctgaacaatcacct |
| | atgaattttgcctcgatggcattgcgttgannncctcctgcctccaaacaaa |
| | cattgtgttgacattaagaatgttggtttactttcaaatcggtcccctgtcg |
| | agttcggctctgggtgggcagtaggaccaccagtgtggggttctgctgggac |
| | cttggagagcctgcatcccaggatgctgggtggccctgcagcctcctccacc |
| | tcacctccatgacagcgctaaacgttggtgacggttgggagcctctggggct |
| | gttgaagtcaccttgtgtgttccaagtttccaaacaacagaaagtcattcct |
| | tcctcttaaaatggtgcttaagttccagcagatgccacataaggggtttgcc |
| | atttgatacccctggggaacatttctgtaaataccattgacacatccgcctt |
| | ttgtatacatcctgggtaatgagaggtggcttttgcggccagtattagactg |
| | gagttcatacctaagtactgtaataatacctcaatgtttgaggagcatgcct |
| | cgtatacaaatatattgttaatctctgttatgtactgtactaattcttacac |
| | tgcctgtatactttagtatgacgctgatacataactaaatttgatacttata |
| | ttttcgtatgaaaatgagttgtgaaagttttgagtagatattacctcatcac |
| | ctcttgaacta (SEQ ID NO: 47) |
| | GCCATCGAGGCAAAATTCCCCCATCTGCGTCAGTCCCCTCAACTTCCGCCTC |
| | TACGCGGGCCTCGCGGCAGGGCGGTACGTCAGCCATTCTGGTCCGCCGCGCG |
| | CATGCGTAGTGGAGCTTGCAGGCTGGAGCTnnnnnnnnnnnnnntcnctcan |
| | cttcngcctctacacgggcctcgcggcagggcggtacttcagccattctggt |
| | cctgcccgcagatgcgtatnnnngcttgaatgcaggagctgaacaatcacct |
| | atgaattttgcctcgatggcattgcgttgannncctcctgcctccaaacaaa |
| | cattgtgttgacattaagaatgttggtttactttcaaatcggtcccctgtcg |
| | agttcggctctgggtgggcagtaggaccaccagtgtggggttctgctgggac |
| | cttggagagcctgcatcccaggatgctgggtggccctgcagcctcctccacc |
| | tcacctccatgacagcgctaaacgttggtgacggttgggagcctctggggct |
| | gttgaagtcaccttgtgtgttccaagtttccaaacaacagaaagtcattcct |
| | tcctcttaaaatggtgcttaagttccagcagatgccacataaggggtttgcc |
| | atttgatacccctggggaacatttctgtaaataccattgacacatccgcctt |
| | ttgtatacatcctgggtaatgagaggtggcttttgcggccagtattagactg |
| | gagttcatacctaagtactgtaataatacctcaatgtttgaggagcatgcct |
| | cgtatacaaatatattgttaatctctgttatgtactgtactaattcttacac |
| | tgcctgtatactttagtatgacgctgatacataactaaatttgatacttata |
| | ttttcgtatgaaaatgagttgtgaaagttttgagtagatattacctcatcac |
| | ctcttgaactaagaaacttttgtaaagaaatttactatatatatatgcctct |
| | gc (SEQ ID NO: 48) |

TABLE G

| Designation | Sequence/SEQ ID NO: |
|---|---|
| MYC1-18 | first digestion enhancing sequence: |
| | gcccgcgaggacccgcccgagc (SEQ ID NO: 69) |
| | first restriction sequence: TTCGAA; |
| | first poly A sequence: AAAAAAAA |
| | the promoter sequence: |
| | gccatcgaggcaaaattcccccatctgcgtcagtcccctcaacttccgcctct |
| | acgcgggcctcgcggcagggcggtacgtcagccattctggtccgccgcgcgca |
| | cgctccgggcgccgggttccggcgttgtcagggtccgcggccctacgatgtgg |
| | gcggtgtccaaggctgcgtagtggagcttgcaggctggagct (SEQ ID NO: |
| | 28) |
| | nucleic acid sequence: |
| | gagcaatcacctatgaacttgctgctaagttatgatcaaatgcaacctcacaa |
| | ccttggctgagtcttgagactgaaagatttagccataatgtaaactgcctcaa |
| | attggactttgggcataaaagaaccctcctgcatgcttaccatccctcctgcc |
| | ctcctttaacagcctcgtaacttataattgacctcaaaaaacctcaagattta |
| | cacaatgtttctctgtaaatattgccattaaatgtaaataactttaataaaac |
| | gtttatagcagttacacagaatttcaatcctagtatatagtacctagtattat |
| | aggtactataaaccctaacctcctgcatttaagtacattttgcactgcaaagt |
| | aagttatcctctattgacctcagaaaaaataaaataactggcaaatatatcat |
| | tgagccaaatcttaagttgtgaatgctgcctctgctaacttatccctcccaac |
| | caccaccatccctgctgccctcatcaa (SEQ ID NO: 4) |
| | second poly A sequence: AAAAAAAA; |
| | second restriction sequence: CCTAG |
| | second digestion enhancing sequence: |
| | gggccggccccgccg (SEQ ID NO: 70) |
| ERBB2-30 | first digestion enhancing sequence: |
| | gcccgcgaggacccgcccgagc (SEQ ID NO: 69) |
| | first restriction sequence: TTCGAA |
| | first poly A sequence: AAAAAAAA |
| | promoter: |
| | GCCATCGAGGCAAAATTCCCCCATCTGCGTCAGTCCCCTCAACTTCCGCCTCT |
| | ACGCGGGCCTCGCGGCAGGGCGGTACGTCAGCCATTCTGGTCCGCCGCGCGCA |
| | CGCTCCGGGCGCCGGGTTCCGGCGTTGTCAGGGTCCGCGGCCCTACGATGTGG |
| | GCGGTGTCCAAGGCTGCGTAGTGGAGCTTGCAGGCTGGAGCT (SEQ ID NO: |
| | 28) |
| | nucleic acid sequence: |
| | caggggaacctgccatgccaggaacctgtcctaaggaacctgccttcctgctt |
| | gagttcccagatggctggaaggggtccagcctcgttggaagaggaacagcact |
| | ggggagtcttcgtggattctgaggccctgcccaatgagactctagggtccagt |
| | ggatgccacagcccagcttggccctctccttccagatcctgggtactgaaagc |
| | cttagggaagctggcctgagaggggaagcggccctaagggagtgtctaagaac |
| | aaaagcgacccattcagagactgtccctgaaacctagtactgccccccatgag |
| | gaaggaacagcaatggtgtcagtatccaggtcgcgttcagagtgccctcctgc |
| | ttagctgctaagttatctgcctcgctgcctcaaagatgaaataaagacccagg |
| | gggagaatgggtgttgtatggggaggcaagtgtggggggtccttctccacacc |
| | cactttgtccatttgcaaatatat (SEQ ID NO: 12) |
| | second poly A sequence: AAAAAAAA; |
| | second restriction sequence: CCTAG |
| | second digestion enhancing sequence: |
| | gggccggccccgccg (SEQ ID NO: 70) |
| MYCN1-18 | first poly A sequence: AAAA |
| | the promoter sequence: |
| | GCCATCGAGGCAAAATTCCCCCATCTGCGTCAGTCCCCTCAACTTCCGCCTCT |
| | ACGCGGGCCTCGCGGCAGGGCGGTACGTCAGCCATTCTGGTCCGCCGCGCGCA |
| | TGCGTAGTGGAGCTTGCAGGCTGGAGCTGAGCAATCACCTATGAACTTGCTG |
| | (SEQ ID NO: 29) |
| | nucleic acid sequence: |
| | cctcctgcctccaaacaaacattgtgttgacattaagaatgttggtttacttt |
| | caaatcggtcccctgtcgagttcggctctgggtgggcagtaggaccaccagtg |
| | tggggttctgctgggaccttggagagcctgcatcccaggatgctgggtggccc |
| | tgcagcctcctccacctcacctccatgacagcgctaaacgttggtgacggttg |
| | ggagcctctggggctgttgaagtcaccttgtgtgttccaagtttccaaacaac |
| | agaaagtcattccttcctcttaaaatggtgcttaagttccagcagatgccaca |
| | taaggggtttgccatttgatacccctggggaacatttctgtaaataccattga |
| | cacatccgccttttgtatacatcctgggtaatgagaggtggcttttgcggcca |
| | gtattagactggagttcatacctaagtactgtaataatacctcaatgtttgag |
| | gagcatgcctcgtatacaaatatattgttaatctctgttatgtactgtactaa |
| | ttcttacactgcctgtatactttagtatgacgctgatacataactaaatttga |
| | tacttatattttcgtatgaaaatgagttgtgaaagttttgagtagatattacc |
| | tcatcacctcttgaactaagaaacttttgtaaagaaatttactatatatatat |
| | gcctctgc (SEQ ID NO: 21) |
| | second poly A sequence: AAAAAAA |
| MYCN1-14 | first poly A sequence: AAAA |
| | the promoter sequence: |
| | GCCATCGAGGCAAAATTCCCCCATCTGCGTCAGTCCCCTCAACTTCCGCCTCT |
| | ACGCGGGCCTCGCGGCAGGGCGGTACGTCAGCCATTCTGGTCCGCCGCGCGCA |
| | TGCGTAGTGGAGCTTGCAGGCTGGAGCTGAGCAATCACCTATGAACTTGCTG |
| | (SEQ ID NO: 29) |
| | nucleic acid sequence: |
| | cctcctgcctccaaacaaacattgtgttgacattaagaatgttggtttacttt |
| | caaatcggtcccctgtcgagttcggctctgggtgggcagtaggaccaccagtg |
| | tggggttctgctgggaccttggagagcctgcatcccaggatgctgggtggccc |
| | tgcagcctcctccacctcacctccatgacagcgctaaacgttggtgacggttg |
| | ggagcctctggggctgttgaagtcaccttgtgtgttccaagtttccaaacaac |
| | agaaagtcattccttcctcttaaaatggtgcttaagttccagcagatgccaca |
| | taaggggtttgccatttgatacccctggggaacatttctgtaaataccattga |
| | cacatccgccttttgtatacatcctgggtaatgagaggtggcttttgcggcca |
| | gtattagactggagttcatacctaagtactgtaataatacctcaatgtttgag |
| | gagcatgcctcgtatacaaatatattgttaatctctgttatgtactgtactaa |
| | ttcttacactgcctgtatactttagtatgacgctgatacataactaaatttga |
| | tacttatattttcgtatgaaaatgagttgtgaaagttttgagtagatattacc |
| | tcatcacctcttgaactaagaaacttttgtaaagaaatttactatatatatat |
| | gcctctgc (SEQ ID NO: 21) |
| | second poly A sequence: AAAAAAA |

In particular aspects, a DNA molecule is utilized, the DNA molecule including in the 3′ to 5′ direction: (i) a first digestion enhancing sequence, (ii) a first restriction sequence, (iii) a first poly A sequence, (iv) a promoter sequence, (v) a destabilized sequence (the disclosed destabilized ARE 3′UTRs), (vi) a second polyA sequence, (vii) a second restriction sequence, and (viii) a second digestion enhancing sequence. The DNA molecule may be a DNA molecule that is incorporated into a plasmid vector.

The first digestion enhancing sequence may be selected from established restriction sequences. The first digestion enhancing sequence may be, for example, about 5 to about 100 nucleotides. The first digestion enhancing sequence may be, as a further example, about 5 to about 50 nucleotides.

In one embodiment, the first digestion enhancing sequence consists of or comprises GGACCCGCCCGAGC (SEQ ID NO: 69). In another embodiment, the first digestion enhancing sequence consists of or comprises GGGCCGGCCCCGCCG (SEQ ID NO: 70). In yet another embodiment, the first digestion enhancing sequence consists of or comprises GCCCGCGAGGACCCGCCCGAGC (SEQ ID NO: 71). The first digestion enhancing sequence and the digestion enhancing sequence may be the same sequence.

Similarly, the second digestion enhancing sequence may be selected from established restriction sequences. The second digestion enhancing sequence may be, for example, about 5 to about 100 nucleotides. The second digestion enhancing sequence may be, for example, about 5 to about 50 nucleotides.

In one embodiment, the second digestion enhancing sequence consists of or comprises GGACCCGCCCGAGC (SEQ ID NO: 69). In another embodiment, the second digestion enhancing sequence consists of or comprises GGGCCGGCCCCGCCG (SEQ ID NO: 70). In yet another embodiment, the second digestion enhancing sequence consists of or comprises GCCCGCGAGGACCCGCCCGAGC (SEQ ID NO: 71).

The first restriction sequence may be selected from available sequences. Suitable examples include a BstBl restriction site, a BamHl restriction site, a Xbal restriction site, a Apal restriction site, a PspOMl restriction site, and the like. The first restriction sequence may have, for example, between four and seven residues. The first restriction sequence and the second restriction sequence may be the same sequence or different sequences.

Similarly, the second restriction sequence may be selected from available sequences. Suitable examples include a BstBl restriction site, a BamHl restriction site, and the like. The second restriction sequence may have, for example, between four and seven residues. As noted, the first restriction sequence and the second restriction sequence may be the same sequence or different sequences.

The first poly A sequence may generally have about 4 to about 20 nucleotides. The first polyA sequence can function in halting the RFP transcription. In one embodiment, the first polyA sequence consists of or comprises: AAAA, AAAAAAAA, or more. The first poly A sequence and the second poly A sequence may be the same or different.

Similarly, the second poly A sequence may generally have about 4 to about 20 nucleotides. The second polyA sequence can function in halting transcription. In one embodiment, the second polyA sequence consists of or comprises: AAAA, AAAAAAAA, or more. As noted, the first poly A sequence and the second poly A sequence may be the same or different.

The promoter sequence may be selected from available sequences. Suitable examples include a DCP1A promoter, a DCP2 promoter, and a ZFP36 promoter. The promoter sequence may be, for example, about 140 to about 170 nucleotides. The promoter sequence may be, as a further example, between about 150 to about 160 nucleotides.

As an alternative to the use of restriction sites, Gibson assembly may be utilized in accordance with available methods. See, e.g., Gibson et al., 2009, Nature Methods 6:343-345; Gibson et al., 2010, Nature Methods 7:901-903; Rabe and Cepko, 2020, BioRxiv https://doi.org/10.1101/2020.06.14.150979. Protocols and reagents are generally available from, for example, New England Biolabs, OpenWetWare Gibson assembly, OpenWetWare Matsen guide to Gibson assembly, among others.

The destabilized sequence may be based on RNAs that are associated with cancer but having the resulting RNA as less stable than the wildtype RNA found in the cancer cells. As detailed herein, modification, and, thereby, destabilization is driven by degrading endogenous mRNA coupled with an increase in transcription of the destabilized mRNA construct relative to the wildtype RNA. That is, transcription of the destabilized mRNA is more efficient such that the destabilized 3′UTR outcompetes the wildtype RNA.

Thus, in various aspects, the DNA molecules of the disclosure comprise a nucleic acid sequence that encodes a 3′UTR of a mRNA molecule that is of interest. In the DNA molecule, one or more ARE poly(U) stabilizing motifs of the 3′UTR are substituted with a destabilizing motif. This destabilized ARE of the 3′UTR will be driven by an mRNA decapping protein, e.g., DCP1A. The decapping protein can specifically upregulate the mRNA decay pathway. This can trigger the deadenylase, e.g., CNOT1, and a cleavage enzyme, e.g., XRN1, to degrade the RNA transcript of interest.

In various aspects, the RNA molecule comprises a 3′UTR of an mRNA encoding an ERBB2, MYC, or MYCN protein, in which one or more ARE poly(U) stabilizing motifs of the 3′UTR are substituted with a nucleic acid sequence consisting of or comprising: a destabilizing motif set out in Table A.

In various further aspects, the RNA molecule comprises a 3′UTR of an mRNA encoding an ERBB2, MYC, or MYCN protein, in which at least two ARE poly(U) stabilizing motifs of the 3′UTR are substituted with a nucleic acid sequence consisting of or comprising: a destabilizing motif set out in Table A.

In various further aspects, the RNA molecule comprises a 3′UTR of an mRNA encoding an ERBB2, MYC, or MYCN protein, in which at least three ARE poly(U) stabilizing motifs of the 3′UTR are substituted with a nucleic acid sequence consisting of or comprising: a destabilizing motif set out in Table A.

In various aspects, the RNA molecule comprises a 3′UTR of an mRNA encoding an ERBB2, MYC, or MYCN protein, in which at least four ARE poly(U) stabilizing motifs of the 3′UTR are substituted with a nucleic acid sequence consisting of or comprising: a destabilizing motif set out in Table A.

The one or more ARE poly(U) stabilizing motifs may have at least two consecutive U's. The one or more ARE poly(U) stabilizing motifs may have at least three consecutive U's. The one or more ARE poly(U) stabilizing motifs may have at least four consecutive U's. The one or more ARE poly(U) stabilizing motifs may consist of or comprise: a destabilizing motif set out in Table A.

The destabilizing motif may consist of or comprise the sequence CCUC. The destabilizing motif may consist of or may comprise the sequence CCUCU-S-S, where S is a C or G. The destabilizing motif may consist of or comprise the sequence CCUCCU-S-CCUC (SEQ ID NO: 162), where S is C or G.

For a MYC mRNA, a destabilizing motif may consist of or comprise, for example: CCUC, CUGC, ACCUC, AUUUU, UAAGUUAUG, UAACUUAU, UGCUGCCCU, GUAAAUAG, CCUCUGC, UCCUGCCCUC (SEQ ID NO: 50), CCUCGUAACUU (SEQ ID NO: 52), CCUCCUGCAUUU (SEQ ID NO: 54), UAAGUUAUCCUCUAUU (SEQ ID NO: 57), CUGCCUCUGCUAACUUAU (SEQ ID NO: 58), or CCUCCUGCCUC (SEQ ID NO: 53).

For an ERBB2 mRNA, a destabilizing motif may consist of or may comprise, for example: UU, UCU, CCUC, CUGC, AUUU, CGCGU, UUCGU, UGCCUU, ACCUC, AUUIUU, UAAGUUAU, UAACUUAU, GUAAAUAG, CCUCUGC, CCUCCUGCUUA (SEQ ID NO: 51), CUGCUAAGUUAUCU (SEQ ID NO: 56), CCUCGCUGCCUC (SEQ ID NO: 55), or CCUCCUGCCUC (SEQ ID NO: 53).

For a MYCN mRNA, a destabilizing motif may consist of or may comprise, for example: CCUC, CUGC, ACCUC, AUUUU, CCUCUGC, UAAGUUAU, UAACUUAU, GUAAAUAG, or CCUCCUGCCUC (SEQ ID NO: 53).

In various aspects, at least two ARE poly(U) stabilizing motifs of the 3′UTR are substituted with a destabilizing motif. In a further aspect, at least four ARE poly(U) stabilizing motifs of the 3′UTR are substituted with a destabilizing motif. In a still further aspect, at least four ARE poly(U) stabilizing motifs of the 3′UTR are substituted with a destabilizing motif. In various aspects, each ARE poly(U) stabilizing motif of the 3′UTR of the mRNA is substituted with a destabilizing motif.

In various aspects, the DNA molecules of the disclosure contain various components (e.g., a promoter, a nucleic acid sequence encoding a 3′UTR in a mRNA molecule, a first and/or second polyA sequence, a first and/or second restriction sequence), which components can be operatively linked to one another in order to provide a DNA construct useful as a therapeutic component as further described herein.

### Methods of Production

This disclosure provides methods for making a DNA molecule (e.g., a complementary deoxyribonucleic acid or cDNA molecule), the method comprising reverse transcribing a RNA molecule of the disclosure to produce the DNA molecule. In a further aspect, the DNA molecule is a cDNA molecule.

In one aspect, disclosed are DNA molecules (e.g., cDNA molecules) prepared from a disclosed RNA molecule. It is understood that cDNA is distinct from genomic DNA, as the derivative template RNA transcript lacks promoters and introns. It is understood also that cDNA can be synthesized via reverse transcription.

In reverse transcription, mRNA or miRNA is used as a template together with a reverse transcription enzyme and a thermostable primer that is complementary to the 3′ end of the RNA template to generate a cDNA product that is a complementary copy of the mRNA. This cDNA product can then be used as a template to produce a second DNA strand using polymerase chain reaction (PCR) assays.

Methods of reverse transcription are widely used and available; see, e.g., Sissaoui et al. (2020) Circ. Res. 126(7): 875-888. Reverse transcription kits are also widely available (see, for example, QIAGEN reverse transcription kit, catalog no. 205311) and can be used according to the manufacturer's protocol.

Briefly, the RNA samples (e.g., a RNA molecule as disclosed herein) can be prepared and any remaining genomic DNA can be removed. The sample can then be combined with the reverse transcriptase enzyme and various other components (e.g., dNTPs, DTT, buffer, RNAse inhibitors, RNase-free water), followed by primer annealing, DNA polymerization, and enzyme activation. Subsequently, the DNA can be incorporated into a vector and then, transfected into cells.

In this way, it is possible to obtain cDNA comprising, in the 5′ to 3′ direction of transcription, a promoter, operatively linked to a nucleic acid sequence encoding a 3′UTR of an ERBB2, MYC, or MYCN gene in the mRNA molecule in which one or more ARE poly(U) stabilizing motifs of the 3′UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein, can be generated.

Thus, in various aspects, the RNA molecule of this disclosure can include, in addition to the 3′UTR of an mRNA encoding an ERBB2, MYC, or MYCN protein, additional sequences that may or may not code for the protein. In various further aspects, the RNA molecule of the disclosure is just the 3′UTR of an mRNA encoding an ERBB2, MYC, or MYCN protein.

Accordingly, a DNA molecule of this disclosure may consist of or comprise one or more of: SEQ ID NO: 4-27. Where a promoter sequence is included, a DNA molecule of this disclosure may consist of or comprise one or more of: SEQ ID NO: 30-48. Variant DNA molecules are also encompassed (e.g., having one or more nucleotides deleted, added, or transposed).

In specific aspects, the DNA molecule of this disclosure may encode a 3′UTR of an ERBB2, MYC, or MYCN mRNA which is a variant having at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 4-27.

In specific aspects, the DNA molecule of this disclosure may encode a 3′UTR of an ERBB2, MYC, or MYCN mRNA which is a variant having the destabilizing motifs as disclosed (e.g., 100% identity to the disclosed motif(s)) and, for the remainder of the nucleotide sequence, having at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 4-27.

In other aspects, the DNA molecule of the disclosure may comprise a nucleic acid encoding a 3′UTR of an ERBB2, MYC, or MYCN mRNA, in which at least two, at least three, or at least four ARE poly(U) stabilizing motifs of the 3′UTR are substituted with a destabilizing motif to produce a nucleotide sequence which has at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 4-27.

In other aspects, the nucleic acid may have at least two, at least three, or at least four destabilizing motifs as disclosed (e.g., 100% identity to the disclosed motif(s)) and the remainder of the nucleotide sequence may have at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 4-27.

In various aspects, the DNA molecule of the disclosure is prepared by reverse transcribing a RNA molecule consisting essentially of a 3′UTR of an mRNA encoding an ERBB2, MYC, or MYCN protein, in which at least two, at least three, or at least four ARE poly(U) stabilizing motifs of the 3′UTR are substituted with a nucleic acid which is at least at least 50%, at least 60%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 4-27.

Provided also are plasmid vectors comprising a DNA molecule of this disclosure. The vector can be any vehicle for carrying a nucleic acid that can, for example, enable said nucleic acid to be introduced into prokaryotic and/or eukaryotic host cells and, where appropriate, to be integrated into a genome. The vector may be replicated and/or expressed in the cell. Vectors can include plasmids, phagemids, and vims genomes, amongst others. Specifically noted are lentiviral plasmid vectors.

The vector can include transgene insert and an origin of replication, a promoter region, optionally a selectable marker, and convenient restriction sites. Features can be included to allow for semi-independent replication of the plasmid in the host (e.g., at least hundreds of copies made per cell) and convenient restriction sites.

Vectors derived from retroviruses such as the lentivirus (e.g., a lentiviral vector) are suitable tools to achieve long-term gene transfer. Such vectors allow long term, stable integration of a transgene and its propagation in daughter cells. As a noted advantage, retroviral vectors can transduce non-proliferating cells, such as hepatocytes. Additionally, they are non-toxic to target cells. In in vivo applications, lentiviral vectors have the added advantage of low immunogenicity. Moreover, lentiviral vectors can deliver genes to cell types that previous retrovirus vectors could not, such as neurons, lymphocytes, and macrophages.

Lentiviruses represent a genus of slow viruses of the Retroviridae family, which includes the human immunodeficiency viruses (HIV), the simian immunodeficiency virus (SIV), the equine infectious encephalitis virus (EIAV), the caprine arthritis encephalitis virus (CAEV), the bovine immunodeficiency virus (BIV), and the feline immunodeficiency virus (FIV). Lentiviruses can persist indefinitely in their hosts and replicate continuously at variable rates during the course of the lifelong infection. Persistent replication of the viruses in their hosts relates to their ability to circumvent host defenses.

The design of recombinant integrating lentiviral vectors takes into account the separation of the cis- and trans-acting sequences of the lentivirus. Efficient transduction in non-dividing cells involves the presence of two cis-acting sequences in the lentiviral genome, the central polypurine tract (cPPT), and the central termination sequence (CTS). These lead to the formation of a triple-stranded DNA structure called the central DNA “flap”. This, in turn, maximizes the efficiency of gene import into the nuclei of non-dividing cells, including dendritic cells (DCs). See, e.g., Zennou et al. (2000) Cell 101(2) 173-85; Arhel et al. (2007) EMBO J 26(12): 3025-37.

A component of the integration complex of LV is the viral integrase enzyme (IN) that catalyzes viral DNA integration into the host genome. This enzyme mediates the integration between vector and host DNA. There are also alternatives to vector-mediated integration. For example, non-integrating lentiviral vectors (NILVs) have been established. NILVs can stably express transgenes from the extrachromosomal DNA in non-dividing cells or transiently if the target cells divide both in vitro and in vivo.

Lentiviral particles containing lentiviral vectors can be produced, by example, by recombinant technology upon transient transfection of cells (e.g., HEK 293 T human cultured cells) by a plasmid DNA such as, for example, a plasmid vector of the disclosure. In this way, transient production of lentiviral particle vectors can be obtained from the transfected cells. Alternatively, lentiviral particle vectors can also be continuously produced by cells by stably inserting the packaging genes, the plasmid DNA (e.g., a DNA plasmid of this disclosure), and the envelope gene into the cellular genome. This allows for the continuous production of lentiviral particle vectors by the cells without the need for transient transfection. It is also possible to use a combination of these procedures, as would be understood by those of ordinary skill.

It will be understood that different vectors may be employed in the methods of this disclosure. In addition methods for constructing vectors, including the choice of an appropriate vector, and the cloning and expression of a polynucleotide sequence inserted into an appropriate vector as described above is believed to be within the capabilities of a person of skill in the art. The skilled person recognises that there are many suitable alternative systems and methods available that may be used to obtain the RNA and DNA molecules disclosed herein. See, e.g., Green and Sambrook, Molecular Cloning: A Laboratory Manual, 4^{th} Edition, Cold Spring Harbour Laboratory Press.

The plasmid vectors disclosed herein can be formulated for administration according to a variety of different techniques, which are widely available. For example, the disclosed plasmid vector can be formulated for administration via a lipid nanoparticle, nanodiamonds, liposomes, microspheres, polymeric micelles, GalNac-conjugation, as a dextran formulation, as a polyethylene glycol (PEG) formulation, as an exosome formulation, and any other similar formulation know to those of ordinary skill. This is set out in more detail below.

### Therapeutics

### Nanoparticle Formulations

Provided are nanoparticle or nanoparticulate formulations for administration of the polynucleotides or vectors of the present disclosure. Nanoparticulate carriers such as lipid carriers that are contemplated include any substances or vehicles with which a nucleic acid such as DNA can be associated. Such association can be made by forming complexes with the nucleic acid or forming vesicles in which the nucleic acid is enclosed or encapsulated. This can result in increased stability of the nucleic acid compared to a naked nucleic acid. In particular, stability of the nucleic acid in blood may be increased.

In certain aspects, nanoparticulate compositions are dispersions or emulsions (e.g., combinations of at least two immiscible materials). In various aspects, the ratio of DNA, (e.g., a DNA molecule of this disclosure), to lipid particle is about 1:2 to about 2:1, or about 1:1.5 to about 1.5:1, or about 1:1.

Nanoparticulate nucleic acid preparations can be obtained by various protocols and from various nucleic acid complexing compounds. Lipids, polymers, oligomers, and amphipiles are typical complexing agents. In various aspects, the complexing compound comprises at least one agent selected from the group consisting of: protamine, polyethyleneimine, a poly-L-lysine, a poly-L-arginine, and a histone.

Nanoparticles can further include a neutral lipid in view of structural stability and the like. The neutral lipid can be appropriately selected in view of the delivery efficiency of the nucleic acid-lipid complex. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, sterol, and cerebroside.

In the case where a cationic liposome includes both a cationic lipid and a neutral lipid, the molar ratio of the cationic lipid to the neutral lipid can be appropriately determined in view of stability of the liposome and the like. See, e.g., Piotrowski-Daspit et al. (2020) Advanced Drug Delivery Reviews 156: 119-132; Kulkami et al. (2021) Nature Nanotechnology 16: 630-643; Zhong et al. (2023) Nature Materials 22: 818-831.

According to one aspect, the nanoparticles described herein can comprise phospholipids. The phospholipids can be a glycerophospholipid. Examples of glycerophospholipid include, but are not limited to: (i) zwitterionic phospholipids, which include, for example, phosphatidylcholine (PC), egg yolk phosphatidylcholine, soybean-derived PC in natural, partially hydrogenated or fully hydrogenated form, dimyristoyl phosphatidylcholine (DMPC) sphingomyelin (SM); (ii) negatively charged phospholipids: which include, for example, phosphatidyl serine (PS), phosphatidylinositol (PI), phosphatidic acid (PA), phosphatidylglycerol (PG) dipalmipoyl PG, dimyristoyl phosphatidylglycerol (DMPG); synthetic derivatives in which the conjugate renders a zwitterionic phospholipid negatively charged such is the case of methoxy-polyethylene, glycol-distearoyl phosphatidylethanolamine (mPEG-DSPE); and (iii) cationic phospholipids, which include, for example, phosphatidylcholine or sphingomyelin of which the phosphomonoester was O-methylated to form the cationic lipids.

Association of nucleic acid to the lipid carrier can occur, for example, by the nucleic acid filling interstitial spaces of the carrier, such that the carrier physically entraps the nucleic acid, or by covalent, ionic, or hydrogen bonding, or by means of adsorption by non-specific bonds. Whatever the mode of association, it will be understood that the nucleic acid retains the therapeutic properties.

### Liposomal Formulations

Provided are liposomal formulations for administration of the polynucleotides or vectors of the present disclosure. Different types of liposomes can be employed in the context of the present disclosure, including, but not limited to, multilamellar vesicles (MLV), small unilamellar vesicles (SUV), large unilamellar vesicles (LUV), sterically stabilized liposomes (SSL), multivesicular vesicles (MV), and large multivesicular vesicles (LMV), as well as other bilayered forms.

For formation of nucleic acid lipoplexes from nucleic acid and liposomes, any suitable method of forming liposomes can be used so long as it provides the envisaged nucleic acid lipoplexes. Liposomes may be formed using standard methods such as, for example, the reverse evaporation method (REV), the ethanol injection method, the dehydration-rehydration method (DRV), sonication, and other suitable methods. After liposome formation, the liposomes can be sized to obtain a population of liposomes having a substantially homogeneous size range. See, e.g., Piotrowski-Daspit et al. (2020) Advanced Drug Delivery Reviews 156: 1 19-132; Kulkarni et al. (2021) Nature Nanotechnology 16: 630-643; Zhong et al. (2023) Nature Materials 22: 818-831.

The size and lamellarity of the liposome will depend on the manner of preparation and the selection of the type of vesicles to be used will depend on the preferred mode of administration. There are several other forms of supramolecular organization in which lipids can be present in an aqueous medium, comprising lamellar phases, hexagonal and inverse hexagonal phases, cubic phases, micelles, and reverse micelles composed of monolayers. These phases can also be obtained in the combination with DNA or RNA, and the interaction with RNA and DNA can substantially affect the phase state. The described phases can be present in the nanoparticulate nucleic acid formulations of the present disclosure. See, e.g., Piotrowski-Daspit et al. (2020) Advanced Drug Delivery Reviews 156: 119-132; Kulkarni et al. (2021) Nature Nanotechnology 16: 630-643; Zhong et al. (2023) Nature Materials 22: 818-831.

Bilayer-forming lipids typically have two hydrocarbon chains, particularly acyl chains, and a head group, either polar or nonpolar. Bilayer-forming lipids are either composed of naturally-occurring lipids or of synthetic origin, including the phospholipids, such as phosphatidylcholine, phosphatidylethanolamine, phosphatide acid, phosphatidylinositol, and sphingomyelin, where the two hydrocarbon chains are typically about 14 to about 22 carbon atoms in length, and have varying degrees of unsaturation. Other suitable lipids for use in the composition of the present disclosure include glycolipids and sterols such as cholesterol and its various analogs which can also be used in the liposomes.

Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to, 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropy 1-1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA).

Cationic lipids, cationic polymers, and other substances with positive charges can form complexes with negatively charged nucleic acids. These cationic molecules can be used to complex nucleic acids, thereby forming, e.g., so-called lipoplexes or polyplexes, respectively. These complexes have been shown to deliver nucleic acids into cells.

### Drug Delivery Systems

Provided are drug delivery systems for administration of the polynucleotides or vectors of the present disclosure. In various aspects, a drug delivery system may comprise: (a) an inorganic nanocage comprising palladium, iron oxide, or gold (for example, the inorganic nanocage may have a diameter of 15 nm or less), loaded with (b) a plasmid vector comprising a therapeutically effective amount of a DNA molecule.

For example, the DNA molecule may comprise in the 5′ to 3′ direction of transcription relative to synthesis of a messenger RNA (mRNA) molecule comprising a 3′ UTR of an mRNA encoding an ERBB2, MYC, or MYCN protein. This in turn, may comprise: (i) a promoter, operatively linked to (ii) a nucleic acid sequence encoding a 3′ UTR of an ERBB2, MYC, or MYCN gene in the mRNA molecule in which one or more ARE poly(U) stabilizing motifs of the 3′ UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

Nanocages may be prepared by the methods described herein as well as other available methods. DHCA is a base coating directly in nanocages. The end group of DHCA is carboxylic acid, which can be conveniently conjugated with dextran or polyethylene glycol (PEG) as amine group of PEG/dextran can be covalently conjugated with the carboxylic acid with the established protocol.

In one aspect, a drug delivery system may comprise: an inorganic nanocage comprising palladium, iron oxide, or gold, wherein the inorganic nanocage has a diameter of 15 nm or less, loaded with a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding an ERBB2, MYC, or MYCN protein: a promoter, operatively linked to a nucleic acid sequence encoding a 3′ UTR of an ERBB2, MYC, or MYCN gene in the mRNA molecule in which one or more ARE poly(U) stabilizing motifs of the 3′ UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

In one aspect, the inorganic nanocage comprises iron oxide. In another aspect, the inorganic nanocage is an iron oxide nanocage. The drug delivery system may further comprise a polymer shell that surrounds the inorganic nanocage. The polymer shell may further comprise one or more biocompatible polymers. The one or more biocompatible polymers may be dextran (e.g. the polymer shell is a dextran polymer shell). The polymer shell may be capped with a functional organic molecule. The functionalized organic molecule may be a catechol or 3-(3,4-dihydroxyphenyl)propionic acid (DHCA). The inorganic nanocage may be covalently attached to the polymer shell. The polymer shell may have a diameter of 50 nm or less. A pharmaceutical composition comprising the drug delivery system of claim 1 and a pharmaceutically effective carrier.

In one aspect, a method of treating cancer in a subject in need thereof is provided, the method comprising administering to the subject a therapeutically effective amount of a pharmaceutical composition. The subject may be a mammal, including a human.

### Pharmaceutical Compositions

Provided are pharmaceutical compositions for administration of the polynucleotides or vectors of the present disclosure. In one aspect, a pharmaceutical composition may comprise a plasmid vector of this disclosure and a pharmaceutically acceptable carrier. In one aspect, a pharmaceutical composition may comprise a drug delivery system of this disclosure and a pharmaceutically effective carrier. Pharmaceutical compositions may be sterile and may contain an effective amount (e.g., a therapeutically effective amount) of the nucleic acid (e.g., a DNA molecule of this disclosure encoding for a destabilizing ERBB2 or MYC or MYCN 3′UTR).

Pharmaceutical compositions can be prepared in a uniform dosage form and can be prepared using available methods. The pharmaceutical composition can, for example, be in the form of a solution or suspension. Pharmaceutically acceptable carriers include, for example, sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate.

Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents, and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or dispersed in sterile water or other sterile injectable media just prior to use. Suitable inert carriers can include sugars such as lactose. In particular aspects, at least 95% by weight of the particles of the active ingredient have an effective particle size in the range of 0.01 to 10 micrometers.

The pharmaceutical composition can comprise salts, buffer substances, preservatives, carriers, diluents, and/or excipients, all of which are preferably pharmaceutically acceptable. Salts that are not pharmaceutically acceptable can yet be used for preparing pharmaceutically acceptable salts and are included in this disclosure. Pharmaceutically acceptable salts of this kind comprise, in a non-limiting way, those prepared from the following acids: hydrochloric, hydrobromic, sulfuric, nitric, phosphoric, maleic, acetic, salicylic, citric, formic, malonic, succinic acids, and the like. Pharmaceutically acceptable salts can also be prepared as alkali metal salts or alkaline earth metal salts, including, but not limited to, sodium salts, potassium salts, and calcium salts.

Suitable buffer substances for use in the disclosed pharmaceutical composition include, but are not limited to, acetic acid in a salt, citric acid in a salt, boric acid in a salt, and phosphoric acid in a salt. Suitable preservatives for use in the disclosed pharmaceutical composition include, but are not limited to, benzalkonium chloride, chlorobutanol, paraben, and thimerosal. Possible carrier substances for parenteral administration include, but are not limited to, sterile water, glucose solutions, Ringer, Ringer lactate, sterile sodium chloride solution, polyalkylene glycols, hydrogenated naphthalenes and, in particular, biocompatiblc lactide polymers, lactidc/glycolidc copolymers, and polyoxyethylene/polyoxy-propylene copolymers. Exemplary excipients include, but are not limited to, carriers, binders, lubricants, thickeners, surface active agents, preservatives, emulsifiers, buffers, flavoring agents, and colorants.

The pharmaceutical compositions described herein can be administered via any conventional route including, but not limited to, parenteral administration including by injection or infusion. Administration is preferably parenterally, e.g., intravenously, intraarterially, subcutaneously, in the lymph node, intradermally, or intramuscularly, although alternative routes of administration e.g., oral administration, intraperitoneal, subcutaneous, transuretheral, transperineal, transrectal) are also envisioned.

The molecules, vectors, and compositions disclosed herein are preferably administered in effective amounts. In the case of treatment of a particular disease or of a particular condition, the desired reaction preferably relates to inhibition of the course of the disease or condition, or reducing the severity of the disease or condition. Administration of the pharmaceutical composition may act in slowing down the progress of the disease or condition and, in particular, interrupting or reversing the progress of the disease or condition. The desired reaction in a treatment of a disease or of a condition can also be delay of the onset or prevention of the onset of said disease or said condition.

The desired therapeutic result may include CS or complete response, PR or partial response, PFS or progression free survival, OS or overall survival. It may also be possible to resolve or reduce undesired symptoms of the disease or condition. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors acknowledged in the medical arts. For example, the skilled person may start doses of a compound at levels lower than those required to achieve the desired therapeutic effect and to gradually increase the dosage until the desired effect is achieved. If desired, the effective daily dose can be divided into multiple doses for purposes of administration.

An effective amount of an agent or composition described herein will depend on a variety of factors including, but not limited to, the condition to be treated, the severeness of the disease, the individual parameters of the patient, including age, physiological condition, size, and weight, the duration of treatment, the type of an accompanying therapy (if present), the specific route of administration, and other similar factors. Accordingly, the doses administered of the agents, molecules, vectors, and compositions described herein may depend on several of these parameters. In the case that a reaction in a patient is insufficient with an initial dose, higher doses (or effectively higher doses achieved by a different, more localized route of administration) can be used.

In various aspects, the plasmid vector comprises a disclosed DNA molecule of the disclosure. Plasmid DNA vectors can be used as either a preventative or therapeutic DNA vaccine for a wide range of indications, from viral, bacterial, and parasitic disease to cancer and as gene therapy products. See, e.g., Williams et al. (2009) Biotechnol. Adv. 27(4): 353-370. Processes for manufacturing plasmid DNA (pDNA) are available to the skilled person. See, e.g., Williams et al. (2009). Briefly, E. coli cells expressing the plasmid (e.g., a plasmid vector of the invention) may be fermented before being harvested by, for example centrifugation or microfiltration tangential flow filtration (MF-TFF).

The cell membrane may be broken down (e.g., via cell lysis) to reveal a mixture of cellular contents including the plasmid DNA (e.g., a DNA molecule of the invention), genomic DNA, proteins, RNA, and other cell debris. Contaminants may be removed (e.g., via precipitation or flocculation) as is any solid content that arose during the chemical lysis and neutralization of the feed stream. The plasmid may be further purified using, for example, anion exchange chromatography, hydrophobic interaction chromatography, and/or size exclusion chromatography.

The plasmid may then be separated (e.g., via ultrafiltration or diafiltration), concentrated, washed, and resuspended in an appropriate buffer. Finally, sterile filtration or other means may be used to remove microbial contaminants that may have been introduced via processing. Once the pDNA satisfies quality specifications set by regulatory agencies, it can be packaged into a vaccine (e.g., as a pharmaceutical composition of the invention) and subsequently introduced into a patient by, for example, intramuscular injection or particle bombardment. See, e.g., Mor (1998) Biochemical Pharmacology 55(8): 1151-1153.

### Methods and Uses

### Cancer Therapies

This disclosure provides therapeutic agents for targeting ERBB2, MYC, or MYCN in cancers (e.g., solid tumors), including for example, breast cancer (e.g., trastuzumab resistant breast cancers, triple negative breast cancer, for example, expressing MYC/STAT5A/5B), cervical cancer, gastrointestinal cancer, colon cancer, colorectal cancer, sarcoma (e.g., a cholangiosarcoma, a rhabdomyosarcoma, an osteosarcoma), carcinoma (e.g., a hepatocellular carcinoma), brain cancer, skin cancer, prostate cancer (e.g., neuroendocrine prostate cancer), ovarian cancer, lung cancer (e.g. non-small cell lung carcinoma), thyroid cancer, testicular cancer, pancreatic cancer, liver cancer, endometrial cancer, melanoma, blastoma (e.g., retinoblastoma, medulloblastoma, neuroblastoma, hepatoblastoma, nephroblastoma, pancreatoblastoma, pleuropulmonary blastoma) glioma (e.g., astrocytoma, oligodendroglioma, oligoastrocytoma, glioblastoma), hematological cancer, leukemia, lymphoma, chronic myeloproliferative disorder, myelodysplastic syndrome, myeloproliferative neoplasm, plasma cell neoplasm (e.g., myeloma) and osteosarcoma.

Noted in particular are therapies for a gastric adenoma, breast adenocarcinoma, lung squamous carcinoma, lung adenocarcinoma, hepatocellular carcinoma, esophagus carcinoma, hepatocellular carcinoma, gastric carcinoma, colon adenocarcinoma, pancreatic adenocarcinoma, pancreatic cystadenocarcinoma, pancreatic acinar cell carcinoma, serous ovarian carcinoma (e.g., high grade serous ovarian carcinoma), ovarian adenocarcinoma, bladder adenocarcinoma, uterine carcinosarcoma, prostate adenocarcinoma, endometrial adenocarcinoma, rhabdomyosarcoma (e.g., metastatic form), neuroblastoma (e.g., metastatic resistant form), Wilms tumor, retinoblastoma, medulloblastoma, acute myeloid leukaemia, diffuse B cell lymphoma and osteosarcoma. Other representative diseases/disorders are set out herein.

In one aspect, disclosed are methods of reducing ERBB2 expression in a cell expressing ERBB2, the method comprising transfecting the cell with an amount of a vector of the invention to cause a reduction of ERBB2 expression.

In one aspect, the methods of the invention include reducing MYC expression in a cell expressing MYC, the method comprising transfecting the cell with an amount of a vector according to the invention to cause a reduction of MYC expression.

In one aspect, the methods of the invention include reducing MYCN expression in a cell expressing MYCN, the method comprising transfecting the cell with an amount of a vector according to the invention to cause a reduction of MYCN expression.

The cell may be selected from any cell that can be transformed or transfected with an exogenous nucleic acid. Particular preference is given to mammalian cells including, but not limited to, cells from humans, mice, hamsters, pigs, goats, and primates. The cells can be derived from a multiplicity of tissue types and include primary cells and cell lines. Exemplary cells include, but are not limited to, keratinocytes, peripheral blood leukocytes, bone marrow stem cells, and embryonic stem cells. A nucleic acid (e.g., a DNA molecule) can be present in the cell in a single or in several copies and, in various aspects, is expressed in the cell.

In various aspects, the disclosed DNA molecules can be administered to a patient by ex vivo methods. Such methods can include, for example, removing cells from a patient, genetically modifying said cells, and reintroducing the modified cells into the patient. Transfection and transduction methods are widely used and available. A suitable method will include introduction of a nucleic acid into a cell or the uptake of a nucleic acid by such cell, wherein the cell can be present in a subject, e.g., a patient.

In accordance with this disclosure, a cell for transfection of a nucleic acid according to the invention described herein can be present in vitro or in vivo. For example, the cell can form part of an organ, a tissue, and/or an organism. Transfection can be transient or stable. For some applications of transfection, it is sufficient if the transfected genetic material is only transiently expressed. Cells allowing episomal amplification of nucleic acids can greatly reduce the rate of dilution. If it is desired that the transfected nucleic acid actually remains in the genome of the cell and its daughter cells, a stable transfection can be employed.

Any technique useful for introducing, e.g., transferring or transfecting, nucleic acids into cells can be used. DNA may be transfected into cells by standard techniques. Such techniques include, but are not limited to, electroporation, lipofection, and microinjection. In various aspects, DNA may be introduced into cells by electroporation. Electroporation or electropermeabilization relates to a significant increase in the electrical conductivity and permeability of the cell plasma membrane caused by an externally applied electrical field. Such methods can be used for introducing some substance into a cell. Introduction of nucleic acid encoding a 3′ UTR encoding an ERBB2, MYC, or MYCN gene in a mRNA molecule in which one or more ARE poly(U) stabilizing motifs are destabilized, as detailed further herein, results in transcription of a mRNA molecule that is destabilized compared to a wildtype mRNA molecule expressing a 3′ UTR encoding an ERBB2, MYC, or MYCN protein.

In various aspects, the cell may be mammalian. In a further aspect, the cell may be human. In various aspects, the cell has been isolated from a mammal (e.g., human) prior to the transfecting step. In various aspects, the cell is a cancer cell. In a further aspect, the cancer cell is a brain cancer cell, a breast cancer cell, an ovarian cancer cell, a pancreatic cancer cell, a prostate cancer cell. In a still further aspect, the cancer cell is trastuzumab-resistant. In yet an even further aspect, the cancer cell is a triple negative breast cancer cell. In an even further aspect, the cancer cell is a colon cancer cell, a lung cancer cell, a cervical cancer cell, an endometrial cancer cell, a neuroblastoma cell, a meningioma cell, a melanoma cell, a squamous cell carcinoma cell, a soft tissue cell, a muscle cell, a bone cancer cell, white blood cell cancer, or a throat cancer cell. Also noted are rhabdomyosarcoma cells, osteosarcoma cells, neuroblastoma cells, Wilms tumor cells, retinoblastoma cells, medulloblastoma cells, and other cells from cancers noted herein.

In a still further aspect, the cell is a cancer cell where one or more of ERBB2, MYC, or MYCN are key drivers of pathogenesis. In a still further aspect, the cancer cell is resistant to a chemotherapeutic drug. In yet a further aspect, the chemotherapeutic drug to which resistance has developed is selected from paclitaxel, cisplatin, olaparib, doxorubicin, bevacizumab, trastuzumab, trastuzumab dexrutecan, fluorouracil, methotrexate, capecitabine, carboplatin, cyclophosphamide, oxaliplatin, altretamine, bendamustine, busulfan, chlorambucil, daunorubicin, gemcitabine, idarubicin, ifosfamide, mitoxantrone, cabazitaxel, ceritinib, cladribine, dacarbazine, and docetaxel. In an even further aspect, the cancer cell is resistant to an immunotherapeutic. In a still further aspect, the immunotherapeutic is an anti-PD-1 monoclonal antibody, an anti-PD-L1 monoclonal antibody, or a checkpoint inhibitor. In yet a further aspect, the immunotherapeutic is pembrolizumab, nivolumab, brexucabtagene autoleucel, ado-trastuzumab emtansine, aldesleukin, amivantamab-vmjw, atezolizumab, axicabtagene ciloleucel, bevacizumab, blinatumomab, cetuximab, daratumumab, durvalumab, elotuzumab, gemtuzumab ozogamicin, ipilimumab, mogamulizumab, naxitamab, obinutuzumab, ramucirumab, siltuximab, and trastuzumab. Other representative agents are set out herein.

In various aspects, transfecting is via administration to a mammal. For example, as detailed herein, a DNA molecule of the invention can be cloned into a vector (e.g., a vector of the invention) and, thereafter, transfected into a cell. In various aspects, the cell is in a mammalian subject (e.g., human), in which case transfection can be accomplished by formulating the vector as a pharmaceutical composition (e.g., a pharmaceutical composition of this disclosure) and thereafter administering the pharmaceutical composition to the subject. Thus, transfection of the cell is accomplished by virtue of administering the pharmaceutical composition comprising the plasmid DNA.

### Methods of Reducing ERBB2, MYC, or MYCN Expression

This disclosure provides methods for reducing expression of one or more of ERBB2, MYC, or MYCN in a subject.

In one aspect, disclosed are methods of reducing ERBB2 expression in a subject in need thereof, the method comprising transfecting the cell with an amount of a disclosed vector to cause a reduction of ERBB2 expression.

In one aspect, disclosed are methods of reducing MYC expression in a subject thereof, the method comprising transfecting the cell with an amount of a disclosed vector to cause a reduction of MYC expression.

In one aspect, disclosed are methods of reducing MYCN expression in a subject thereof, the method comprising transfecting the cell with an amount of a disclosed vector to cause a reduction of MYCN expression.

The subject may be a mammal (e.g., human), a fish, a bird, a reptile, or an amphibian. The subject may be a human, non-human primate, horse, pig, rabbit, dog, sheep, goat, cow, cat, guinea pig or rodent. Any means of administration may be utilized by the skilled person. Such means include, for example, intravenous administration, oral administration, transdermal administration, administration by inhalation, nasal administration, topical administration, intravaginal administration, ophthalmic administration, intraaural administration, intracerebral administration, rectal administration, transuretheral administration, transperineal administration, transrectal administration, sublingual administration, buccal administration, and parenteral administration, including injectable such as intravenous administration, intra-arterial administration, intramuscular administration, and subcutaneous intratumoral administration, and intrathecal administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

In various aspects, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

In certain aspects, a DNA molecule for administration comprises in the 5′ to 3′ direction of transcription relative to synthesis of a messenger RNA (mRNA) molecule comprising a 3′ UTR encoding an ERBB2 protein: (a) a promoter, operatively linked to (b) a nucleic acid sequence encoding a 3′ UTR encoding an ERBB2 gene in the mRNA molecule in which one or more ARE poly(U) stabilizing motifs are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein, wherein the mRNA molecule encoded by the DNA molecule is destabilized compared to a wildtype mRNA molecule comprising a 3′ UTR encoding an ERBB2 protein.

In certain aspects, a DNA molecule for administration comprises in the 5′ to 3′ direction of transcription relative to synthesis of a messenger RNA (mRNA) molecule comprising a 3′ UTR encoding a MYC protein: (a) a promoter, operatively linked to (b) a nucleic acid sequence encoding a 3′ UTR encoding a MYC gene in the mRNA molecule in which one or more ARE poly(U) stabilizing motifs are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein, wherein the mRNA molecule encoded by the DNA molecule is destabilized compared to a wildtype mRNA molecule comprising a 3′ UTR encoding a MYC protein.

In certain aspects, a DNA molecule for administration comprises in the 5′ to 3′ direction of transcription relative to synthesis of a messenger RNA (mRNA) molecule comprising a 3′ UTR encoding a MYCN protein: (a) a promoter, operatively linked to (b) a nucleic acid sequence encoding a 3′ UTR encoding a MYCN gene in the mRNA molecule in which one or more ARE poly(U) stabilizing motifs are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein, wherein the mRNA molecule encoded by the DNA molecule is destabilized compared to a wildtype mRNA molecule comprising a 3′ UTR encoding a MYCN protein.

In various aspects, the subject is a mammal. In further various aspects, the subject is a human. In various aspects, administering is via oral, intravenous, intraperitoneal, subcutaneous, intramuscular, intracranial, intraspinal, intrarectal, transperineal, or transurethral administration, or intrathecal administration, or other means. In various aspects, the subject has been diagnosed with a need for inhibition of one or more of ERBB2, MYC, or MYC expression prior to the administering step.

### Treatment or Prevention of Proliferative Conditions

This disclosure provides methods of treating or preventing a proliferative disease or disorder. In one aspect, disclosed are methods of treating cancer in a subject in need thereof, the method comprising administering to the subject a polynucleotide, vector, or pharmaceutical composition as set out herein. In one aspect, disclosed are methods of treating a drug resistant cancer in a subject in need thereof, the method comprising administering to the subject a polynucleotide, vector, or pharmaceutical composition as set out herein. In one aspect, disclosed are methods of treating a malignant tumor in a subject in need thereof, the method comprising administering to the subject a polynucleotide, vector, or pharmaceutical composition as set out herein. In one aspect, disclosed are methods of treating a metastatic tumor in a subject in need thereof, the method comprising administering to the subject a polynucleotide, vector, or pharmaceutical composition as set out herein. In one other aspect, disclosed are methods of preventing metastasis in a subject in need thereof, the method comprising administering to the subject a polynucleotide, vector, or pharmaceutical composition as set out herein.

The malignant properties of cancers differentiate them from benign conditions (e.g., benign tumors), which are self-limited, and do not invade or metastasize. Many cancers form tumors but some, like leukemia, do not. Examples of cancers include, but are not limited to: carcinomas, lymphomas (e.g., Hodgkin's, non-Hodgkin's), blastomas, sarcomas, gliomas and leukemias (e.g., acute lymphoblastic leukemia, acute or chronic lymphocytic leukemia, acute or chronic myeloid leukemia, adult leukemia, childhood leukemia). Further examples of cancers include bone cancer, soft tissue cancer, and muscle cancer (e.g., osteosarcoma, rhabdomyosarcoma), blood cancer, brain cancer and nervous system cancer (e.g., glioma tumors, non-glioma tumors, rhabdoid tumors, astrocytomas, ependymomas, glioblastoma multiforme, medulloblastomas, oligodendrogliomas, hemangioblastomas), lung cancer (e.g., small cell lung cancer, non-small cell lung cancer, mesothelioma), liver cancer, pancreatic cancer (e.g., islet cell tumors, neuroendocrine pancreatic tumors, nonendocrine pancreatic tumors, i.e., exocrine pancreatic tumors), skin cancer (e.g., atypical mole syndrome, basal cell carcinoma, melanoma), cancer of the head or neck, cutaneous or intraocular malignant melanoma.

Further examples of cancers include, but are not limited to: carcinoma of the sexual and reproductive organs, uterine cancer (e.g., endometrial cancer, uterine sarcoma, serous adenocarcinoma, and uterine carcinosarcoma), ovarian cancer (e.g., epithelial ovarian carcinoma, peritoneal carcinoma, germ cell tumors, stromal cell tumors, high grade serous ovarian carcinoma tumors), fallopian tube cancer, breast cancer (e.g., ductal carcinoma, invasive ductal carcinoma, lobular carcinoma, invasive lobular carcinoma, triple negative breast cancer, inflammatory breast cancer, Paget's disease of the breast), prostate cancer, penile cancer, cancer of the esophagus, stomach cancer, cancer of the small intestine, colon cancer, colorectal cancer (e.g., colorectal adenocarcinoma, gastrointestinal carcinoid tumors, colorectal lymphomas, gastrointestinal stromal tumors), rectal cancer, cancer of the anal region, cancer of the endocrine system, cancer of the thyroid gland, cancer of the parathyroid gland, cancer of the adrenal gland, cancer of the eye (e.g., retinoblastoma), sarcoma of soft tissue, cancer of the bladder, cancer of the kidney (e.g., renal cell carcinoma, Wilms' tumor), carcinoma of the renal pelvis, cancers of the nervous system, neoplasms of the central nervous system (CNS), neuroectodermal cancer, spinal axis tumors, meningioma, and pituitary adenoma. Included are cancer metastases.

Administration may comprise a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding an ERBB2 protein: (a) a promoter, operatively linked to (b) a nucleic acid sequence encoding a 3′ UTR of an ERBB2 gene in the mRNA molecule in which one or more ARE poly(U) stabilizing motifs of the 3′ UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

Administration may comprise a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYC protein: (a) a promoter, operatively linked to (b) a nucleic acid sequence encoding a 3′ UTR of an MYC gene in the mRNA molecule in which one or more ARE poly(U) stabilizing motifs of the 3′ UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

Administration may comprise a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

In one aspect, disclosed are methods of treating a cancer in a subject having a metastatic tumor, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding an ERBB2 protein: (a) a promoter, operatively linked to (b) a nucleic acid sequence encoding a 3′ UTR of an ERBB2 gene in the mRNA molecule in which one or more ARE poly(U) stabilizing motifs of the 3′ UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

In one aspect, disclosed are methods of treating a cancer in a subject having a metastatic tumor, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYC protein: (a) a promoter, operatively linked to (b) a nucleic acid sequence encoding a 3′ UTR of a MYC gene in the mRNA molecule in which one or more ARE poly(U) stabilizing motifs of the 3′ UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

In one aspect, disclosed are methods of treating a cancer in a subject having a metastatic tumor, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein: (a) a promoter, operatively linked to (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

In one aspect, disclosed are methods of treating cancer in a subject having disease progression after chemotherapy or other cancer treatment, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding an ERBB2, MYC, or MYCN protein: (a) a promoter, operatively linked to (b) a nucleic acid sequence encoding a 3′ UTR of an ERBB2, MYC, or MYCN gene in the mRNA molecule in which one or more ARE poly(U) stabilizing motifs of the 3′ UTR of the mRNA are substituted with a nucleic acid sequence comprising or consisting of: UCU, AUUU, CCUC, CUGC, AUUUU, UUCGU, ACCUC, CGCGU, UGCCUU, CCUCUGC, UAAGUUAU, UAACUUAU, GUAAAUAG, UAAGUUAUG, UGCUGCCCU, UCCUGCCCUC (SEQ ID NO: 50), CCUCCUGCUUA (SEQ ID NO: 51), CCUCGUAACUU (SEQ ID NO: 52), CCUCCUGCCUC (SEQ ID NO: 53), CCUCCUGCAUUU (SEQ ID NO: 54), CCUCGCUGCCUC (SEQ ID NO: 55), CUGCUAAGUUAUCU (SEQ ID NO: 56), UAAGUUAUCCUCUAUU (SEQ ID NO: 57), CUGCCUCUGCUAACUUAU (SEQ ID NO: 58), or others disclosed herein.

Dosage forms can comprise one or more of the disclosed drugs, or salts, solvates, or polymorphs thereof, in combination with one or more pharmaceutically acceptable excipients. Exemplifications include preservatives, buffers, salines, phosphate buffered salines, amongst others. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxy cholate), solution and/or cryo-stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can include one or more of the disclosed drugs, or salts, solvates, or polymorphs thereof, suspended in sterile saline solution for injection together with a preservative. Other formulations may be prepared in accordance with available methods.

Unit dosage forms may be prepared for administration once daily, twice daily, three times daily, four times daily, five times daily, six times daily, or more, or alternatively, once per week, twice per week, three times per week, every other day, or more. Co-administration of two or more of the polynucleotides of this disclosure is also encompassed. Co-administration of the polynucleotide(s) with one or more other therapeutic agents is also encompassed. These include other cancer treatments as well as treatments for pain, inflammation, seizures, anxiety, depression, etc. The disclosed polynucleotide molecules (e.g., ERBB2, MYC, or MYC plasmids) and any combination thereof can be administered via any suitable route and via any suitable formulation. In some cases, it may be useful to use different routes of administration and/or different formulations in the same subject. Non-limiting exemplifications of routes of administration and formulations for administration are provided herein. Co-administration includes co-formulations (i.e., combination formulations), as well as the simultaneous or sequential administration of separate formulations.

Other agents for treating cancers include but are not limited to: alkylating agents such as cyclophosphamide, melphalan, and temozolomide, and including platinum agents such as carboplatin, cisplatin, and oxaliplatin; antimetabolites such as 5-fluorouracil, 6-mercaptopurine, cytarabine, gemcitabine, and methotrexate; anthracycline agents such as actinomycin-D, bleomycin, daunorubicin, and doxorubicin; mitotic inhibitors such as docetaxel, estramustine, paclitaxel, and vinblastine; topoisomerase inhibitors, such as etoposide, irinotecan, teniposide, and topotecan; steroids such as prednisone, methylprednisolone, and dexamethasone; antibody agents such as alemtuzumab, trastuzumab, ibritumomab tiuxetan, brentuximab vedotin, ado-trastuzumab emtansine, denileukin diftitox, and blinatumomab. Specifically noted are: PARPi agents (e.g., olaparib), TOP2i agents (e.g., epirubicin), taxanes (e.g., paclitaxel), intercalating agents or alkylating agents (e.g., cisplatin), HER2/neu/topoisomerase ADCs (e.g., trastuzumab deruxtecan), EGFRi agents (e.g., osimertinib), VEGFi agents (e.g., bevacizumab), anti-androgen hormone therapies including ARi agents (e.g., abiraterone and enzalutamide) and MYC-Max protein inhibitors (e.g., MYCi975).

The treatment and preventative methods may also be utilized in conjunction with other therapeutic procedures. For example, in conjunction with the disclosed methods, the subject may be treated by one or more of chemotherapy, targeted therapy, immunotherapy (e.g., immune checkpoint inhibitors, cancer vaccines, monoclonal antibodies, etc), adoptive cell transfer, gene therapy, hormone therapy, radiotherapy (e.g., external beam radiation, internal radiation, etc), intervention radiology, photodynamic therapy, hyperthermia, stem cell transplant, bone marrow transplant, surgery (e.g., open surgery, minimally invasive surgery, cryosurgery, laser surgery, etc), or other methods. Where multiple therapeutic methods are being utilized, these may be applied concurrently or sequentially.

In certain aspects, treatment may be carried out using a kit. In particular, a kit may be provided which includes one or more polynucleotides of this disclosure (e.g., ERBB2, MYC, or MYC plasmids). The one or more polynucleotides in the kit may be provided in the form of a composition, for example, a pharmaceutical composition as described herein. The one or more polynucleotides (e.g., formulated as composition(s)) may be provided in one or more containers in the kit. Additional components may also be provided with the kit, for example, one or more excipients, or one or more additional cancer therapeutics (or one or more other therapeutics), intended for use with the one or more compounds. Optionally, instructions may be provided with the kit, as well as any other item, such as any number of containers, labels, or medical tools, including bottles, pads, etc. The instructions for the administration of the pharmaceutical composition may include information as to dosage, dosing schedule, routes of administration, amongst other information. The kit may comprise a description of selecting an individual suitable for treatment or preventative methods based on identifying whether that individual has a malignancy or a symptom of a malignancy or is at risk of having such. The kit may include one or more reagents determining marker expression levels in the subject, and/or localising certain markers in the subject. In this way, the individual may be assessed for the presence of the disorder, and may also be assessed for expected response to administration of the composition.

Also provided are the uses of the disclosed molecules, vectors, pharmaceutical compositions, kits, and attendant products. In one aspect, the invention relates to use of at least one disclosed molecule or at least one disclosed vector. In a further aspect, the molecule or vector is produced by a method disclosed herein.

In various aspects, the use relates to a treatment of proliferative disease or disorder associated with dysregulation of ERBB2, MYC, and/or MYCN. In one aspect, the treatment is carried out in a human. In one aspect, the disease or disorder is cancer.

In a further aspect, a process is provided for preparing a pharmaceutical composition comprising a therapeutically effective amount of one or more of the disclosed molecules or vectors. In one aspect, the disclosed methods are used to obtain a medicament comprising or consisting essentially of the molecule or vector.

In a further aspect, a pharmaceutical composition is prepared to comprise or consist essentially of one or more of the disclosed molecules or vectors. In particular aspects, one or more pharmaceutically acceptable carriers are intimately mixed with a therapeutically effective amount of the molecule(s) or vector(s).

In a further aspect, a method of use is provided for one or more of the disclosed molecules, one or more of the disclosed vectors, one or more of the disclosed pharmaceutical compositions, or one or more of the disclosed kits in the manufacture of a medicament for the treatment of disease or disorder associated with dysregulation of ERBB2, MYC, and/or MYCN signalling in a subject. In a further aspect, the disease or disorder is cancer. Dosing schemes

This disclosure provides dosing schemes for treating or preventing a proliferative disease or disorder. The dosing schemes may be utilized in conjunction with the polynucleotides and pharmaceutical compositions disclosed herein. Specifically noted are dosing schemes for the treatment or prevention of various malignancies as described herein. These include, but are not limited to brain cancers, breast cancers (e.g., triple negative breast cancer), colon cancers, lung cancers (e.g., non-small cell lung cancer), nervous system cancers, pancreatic cancers (e.g., neuroendocrine pancreatic cancer), prostate cancers, and ovarian cancers. Treatment for metastatic tumors and prevention of metastases are also noted.

In particular exemplifications, human clinical testing may be carried out for at least one year (e.g., phase I trials). Based on the IC₅₀ doses determined herein, 3+3 dosing schemes can be employed. In such schemes, three patients are started on the initial dose and a further three patients may be added for each increasing dose. For example, dosing of an mRNA destabilizing drug (e.g., c-MYC vector plasmid) can be started for the initial patient group at the IC₅₀ value. As exemplifications, dosing can be started at 2.5 μg (e.g., for TNBC, CRPC, NEPC) or at 10 μg (e.g., for ovarian cancer) or at 1.88 μg (e.g., for colon cancer). Alternatively, dosing can be started at 21 μg (e.g., for pancreatic cancer) or at 8.32 μg (e.g., uterine cancer) or at 9.8 μg (e.g., for NSCLC) or at 2 μg (e.g., for endometrial cancer).

Similarly, dosing of other mRNA destabilizing drugs (e.g., MYCN vector plasmid) can be started at 10 μg (e.g., for RMS), or at 2.5 μg (e.g., for neuroblastoma). See also exemplary IC₅₀ dosages set out in Table 3A, below. Administration may be by intravenous means or other means as described. For example, intravenous administration of the construct in 5% dextrose saline can be provided 2λ/week (e.g., 2×8 hr infusions). After a continuous dosing period (e.g., 4 weeks), it is possible to include a break to monitor for adverse effects (e.g., 2 weeks). Then, the next group of patients can be recruited and the dosing escalated. This can be repeated until dose limiting toxicity is reached. Exemplary dosage schemes are noted as follows.

For breast cancer patients of African descent, the mRNA destabilizing drug (e.g., MYC mRNA destabilizing drug, such as 3′UTRMYC1-18 or others) can be dosed as follows: (1) 2.5 μg 2λ/week for 4 weeks N=3, and then 2 week break; (2) 5 μg 2λ/week for 4 weeks N=6, and then 2 week break; (3) 10 μg 2λ/week for 4 weeks N=9, and then 2 week break; (4) 20 μg 2λ/week for 4 weeks N=12, and then 2 week break; (5) 40 μg 2λ/week for 4 weeks N=15, and then 2 week break; (6) 80 μg 2λ/week for 4 weeks N=18, and then 2 week break; (7) Increases can be continued until dose limiting toxicity is reached. Noted in particular is treatment of triple negative breast cancer having overexpression of c-MYC.

For breast cancer patients of Caucasian descent, the mRNA destabilizing drug (e.g., MYC mRNA destabilizing drug, such as 3′UTRMYC1-18 or others) can be dosed as follows: (1) 8.7 μg 2λ/week for 4 weeks N=3, and then 2 week break; (2) 17.5 μg 2λ/week for 4 weeks N=6, and then 2 week break; (3) 35.04 μg 2λ/week for 4 weeks N=9, and then 2 week break; (4) 70 μg 2λ/week for 4 weeks N=12, and then 2 week break; (5) 140 μg 2λ/week for 4 weeks N=15, and then 2 week break; (6) 280 μg 2λ/week for 4 weeks N=18, and then 2 week break; (7) Increases can be continued until dose limiting toxicity is reached. Noted in particular is treatment of triple negative breast cancer having overexpression of c-MYC.

For ovarian cancer patients, the mRNA destabilizing drug (e.g., MYC mRNA destabilizing drug, such as 3′UTRMYC1-18 or others) can be dosed as follows: (1) 10 μg 2λ/week for 4 weeks N=3, and then 2 week break; (2) 20 μg 2λ/week for 4 weeks N=6, and then 2 week break; (3) 40 μg 2λ/week for 4 weeks N=9, and then 2 week break; (4) 80 μg 2λ/week for 4 weeks N=12, and then 2 week break; (5) 160 μg 1λ/week for 4 weeks N=15, and then 2 week break; (6) 320 μg 1λ/week for 4 weeks N=18, and then 2 week break; (6) 640 μg 1λ/week for 4 weeks N=21, and then 2 week break; (7) Increases can be continued until dose limiting toxicity is reached. Noted in particular is treatment of pancreatic cancers having overexpression of c-MYC.

For pancreatic cancer patients, the mRNA destabilizing drug (e.g., MYC mRNA destabilizing drug, such as 3′UTRMYC1-18 or others) can be dosed as follows: (1) 21 μg 2λ/week for 4 weeks N=3, and then 2 week break; (2) 31 μg 2λ/week for 4 weeks N=6, and then 2 week break; (3) 43 μg 2λ/week for 4 weeks N=9, and then 2 week break; (4) 54 μg 2λ/week for 4 weeks N=12, and then 2 week break; (5) 64.8 μg 1λ/week for 4 weeks N=15, and then 2 week break; (6) 75.6 μg 1λ/week for 4 weeks N=18, and then 2 week break; (6) 86 μg 1λ/week for 4 weeks N=21, and then 2 week break; (7) Increases can be continued until dose limiting toxicity is reached. Noted in particular is treatment of pancreatic cancers having overexpression of c-MYC.

For brain cancer patients or other nervous system cancer patients, the mRNA destabilizing drug (e.g., MYCN mRNA destabilizing drug such as 3′UTRMYCNM1-14, 3′UTRMYCNM1-18, or others) can be dosed as follows: (1) 10 μg 2λ/week for 4 weeks N=3, and then 2 week break; (2) 20 μg 2λ/week for 4 weeks N=6, and then 2 week break; (3) 40 μg 2λ/week for 4 weeks N=9, and then 2 week break; (4) 80 μg 2λ/week for 4 weeks N=12, and then 2 week break; (5) 160 μg 1λ/week for 4 weeks N=15, and then 2 week break; (6) 320 μg 1λ/week for 4 weeks N=18, and then 2 week break; (7) 640 μg 1λ/week for 4 weeks N=21, and then 2 week break; (8) Increases can be continued until dose limiting toxicity is reached. Noted in particular is treatment of rhabdomyosarcomas, neuroblastomas, and glioblastomas having overexpression of MYCN.

Alternative dosing methods may be utilized with the polynucleotides or compositions of the present disclosure. For example, dosages may range from about 2 μg to about 240 μg, or from about 4 μg to about 320 μg, or from about 10 μg to about 430 μg, or from about 5 μg to about 960 μg. Administration may be, for example, every other week, 1× per week, 2× per week, 3× per week, every other day, 1× per day, 2× per day, 3× per day, or more. Treatments may be continued for at least 2 weeks, at least 3 weeks, at least 4 weeks, at least 5 weeks, at least 6 weeks, 7 weeks, 8 weeks, or more. Breaks from treatments may be taken for at least 5 days, at least 1 week, at least 2 weeks, at least 3 weeks, at least 4 weeks, or more. One or more treatment cycles may be utilized (treatment period plus break). For example, at least 1 cycle, at least 2 cycles, at least 3 cycles, at least 4 cycles, at least 5 cycles, or more may be employed. Combinations of one or more of the disclosed polynucleotides or one or more of the disclosed compositions may be utilized. Combination therapies may be employed using the polynucleotides or compositions of this disclosure along with one or more other therapeutic agents and/or one or more therapeutic procedures as described herein. Dosing schemes can be modified to accommodate particular patients or patient groups, e.g., pediatrics, geriatrics, obese patients, underweight patients, immunocompromised patients, etc.

The examples provided herein are provided for the purpose of illustrating specific embodiments and aspects and are not intended to limit this disclosure in any way. Persons of ordinary skill can utilise the disclosures and teachings herein to produce other embodiments, aspects, and variations without undue experimentation. All such embodiments, aspects, and variations are considered to be part of this disclosure.

### EXAMPLES

### Example 1: Sequence Specific mRNA Transcript Control by Engineered Destabilized 3′UTR is Mediated Via Ribosome Fate Switching

Overview: We developed various 3′UTR mRNA destabilizing drugs which destabilized and degraded specific target mRNA transcript. Here, we report that the mRNA destabilizing drugs are sequence specific in target site mRNA recognition. The in-frame target mRNA recognition sites trigger stalling of ribosome fate switch stronger than the 3′UTR site, which is stronger than the 5′UTR site. The proteins PELO and EXOSC4 in the presence of the destabilized mRNA target recognition trigger the ribosome fate switch from translating to degrading the specific target mRNA. Conclusively, we show that the mRNA destabilizing drugs are sequence specific and recruit the EXOSC4 and PELO proteins which mediates the molecular regulation of ribosome fate switch in various cellular states.

### Example 1A: Materials and Methods

Cell culture: MDAMB231, MDAMB468, NCI H1975, SKOV3, AC16, HEK293T were all obtained from ATCC. MDAMB231, MDAMB468 and HEK293T cells were grown in DMEM media supplemented with the 10% FBS and antibiotics-antimycotics. The NCI H1975 cells were grown in RPMI media supplemented with 10% FBS and antibiotics-antimycotics. The SKOV3 cells were grown in McCoy's 5A media supplemented with 10% FBS and antibiotics-antimycotics. The AC16 cells were grown in DMEM: F12 media supplemented with the supplemented with 10% FBS and antibiotics-antimycotics. All the cells grew until 80% confluency before use and were tested for mycoplasma regularly and were authenticated by short tandem repeat sequencing.

Plasmid system: We obtained the following plasmids for experimental purposes from the Addgene repository under MTA. pYFP-ErBB2 sequence (Addgene ID: 66948) (which expresses the first ERBB2 exon), the pYFP-ERBB2 delta C990 (Addgene ID: 66946) (which expresses the ERBB2 exons 2-7 and intron 2-6) and the pYFP-ERBB2 delta C776 (Addgene ID:66947) (which expresses ERBB2 exons 26-27 and intron 26). The pCL20 mEGFP-MYC-MYC (Addgene ID:205862) expresses 132 bp of c-MYC exon 2. ZsGreen1-cMYC/pLVX-puromycin (Addgene ID: 180278), expresses portion of the c-MYC exon 1, complete exon 2 and portion of the exon 3 and introns 1 and 2. We obtained these various plasmid overexpression cDNA from Origene Inc USA. PELO (Cat ID: SC114538), LSM10 (Cat ID: RC203562), EXOSC4 (Cat ID: RC201058), RPL3 (Cat ID: RC217987).

FACS: To obtain YFP and GFP positive only cells, we expanded the HEK293T cells and transfected them with the different plasmid construct for ERBB2 and c-MYC individually in a 6-well plate, with non-transfected cells as control. After 4 days, we harvested the cells and sorted for the YFP and GFP positive only cells from the ERBB2 and MYC plasmid transduced cells, respectively. These positive cells were then collected and re-expanded until they were ready for the experiment.

We sought to show that the ERBB2 and MYC mRNA destabilizing drugs specifically binds their target sequences on ERBB2 and MYC mRNA, respectively. We split each plasmid transduced HEK293T cells into 3 groups: 1) no treatment, 2) vector treated and 3) desARE3′UTRERBB2-30 (ERBB2 mRNA destabilizing drug) or 3′UTRMYC1-18 (c-MYC mRNA destabilizing drug) treated. One set was un-labelled and the other set were labelled with either antibody HER2/ErbB2 rabbit mAb (29D8-Alexa Fluor 647 conjugate, Cat ID: 12965S) or with antibody c-MYC (ESQW) rabbit mAb (Alexa Fluor 647 conjugate, Cat ID: 45606S) and then analyzed on the BD FACS Diva 9.5.1 flow cytometry machine. The mean fluorescence intensity was plotted in GraphPad Prism.

RNA sequencing: The aim was to determine the gene expression pattern changes and biological pathways mediating the function of the mRNA destabilizing drugs on the target sequences. We extracted the total mRNA from the WT cells, desARE3′UTRERBB2-30, desARE3′UTRERBB2-3 (both targets ERBB2 mRNA), and 3′UTRMYC1-18 (targets c-MYC mRNA) and 3′UTRTEAD1-T5 (targets TEAD1 mRNA). RNA seq library was prepared according to the standard protocol by Azenta Genewiz Inc. This was sequenced on an Illumina Next Gen Seq sequencer. The analysis was performed on BioJupies online RNA seq software.

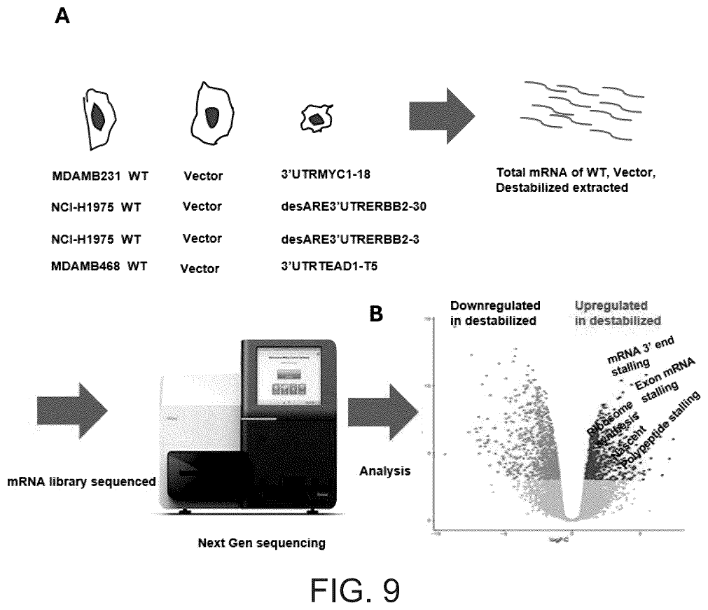

qRT-PCR: To validate the elevated expression of 22 genes (FIG. 9B) implicated as involved in switching the ribosome fate once the 3′UTR mRNA destabilizing drugs bind the target mRNA sequences. We designed qPCR primers (see Table 1A) and purchased them from IDT Inc USA and performed qPCR against the target with the house keeping gene GAPDH. We calculated the fold change in expression as delta CT target-delta CTGAPDH. We plotted the analysis on Graph Pad Prism.

CRISPR Cas9 knockout of PELO, EXOSC4 and RPL11: Briefly, we obtained two sgRNA guides targeting PELO, EXOSC4 and RPL11 (see Table 1B) from the Brunello KO library and purchased them from the Synthego Inc USA. The Cas9a protein was purchased from Thermofisher (Invitrogen Cat: A50574). We set up the gene edit according to Synthego standard protocol and after 4 days assayed for the down regulation of the target gene transcript by qRT-PCR. PELO and EXOSC4 protein sequences are shown in Tables 1C-D, respectively. The RPL3 sequence is shown in Table 1E. The targeted sequences on the c-MYC and ERBB2 transcripts are shown in Tables 1F-G, respectively.

Gain of function mRNA overexpression: To restore the mRNA expression of the CRISPKO genes, we obtained the expression ready cDNA plasmids of PELO, EXOSC4, RPL3 and LSM10 from Origene Inc USA. PELO (Cat ID: SC114538), LSM10 (Cat ID: RC203562), EXOSC4 (Cat ID: RC201058), RPL3 (Cat ID: RC217987). We transfected CRISPRKO EXOSC4, PELO and RPL11 with the over expressing plasmid as one or two, threes, or fours, and assayed for the restoration of the target transcript and cellular phenotypes implicated.

Cell viability: To determine cell viability, we seeded 5000 cells per well in a 96 well plate with controls and the treatment groups. To read the viability of the cells, we used Cell Titre glo (G7570) and obtained the luminescence which measures the ATP. The data was normalized by luminescence of the treated normalized against the controls. All data were plotted in Graph Pad prism.

Western blot: We sought to determine the expression of the ERBB2 proteins in the vectors treated cells, destabilized cells, the cells with CRISPR KO EXOSC4 and PELO and cells with the CRISPR KO and over expressed with EXOSC4 and PELO. We performed a western blot in these cells against the ERBB2/HER2 (Cell Signaling Cat ID: 2165S) and GAPDH (Proteintech Cat ID: 60004-1-Ig) as control.

Microscopy: Graph Pad Prism. The GraphPad Prism software version 10.4.0 was used to plot all the graphs.

Statistical analysis and experimental replicates: All experiments were done at a minimum of N=2. We used the two-tailed T-test to calculate statistical significance between the treated groups and the controls.

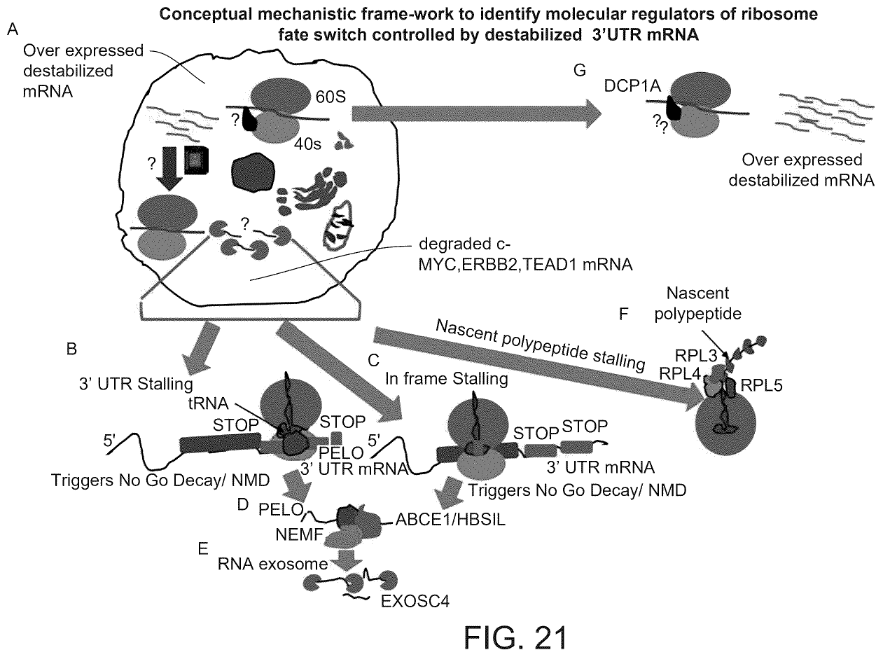

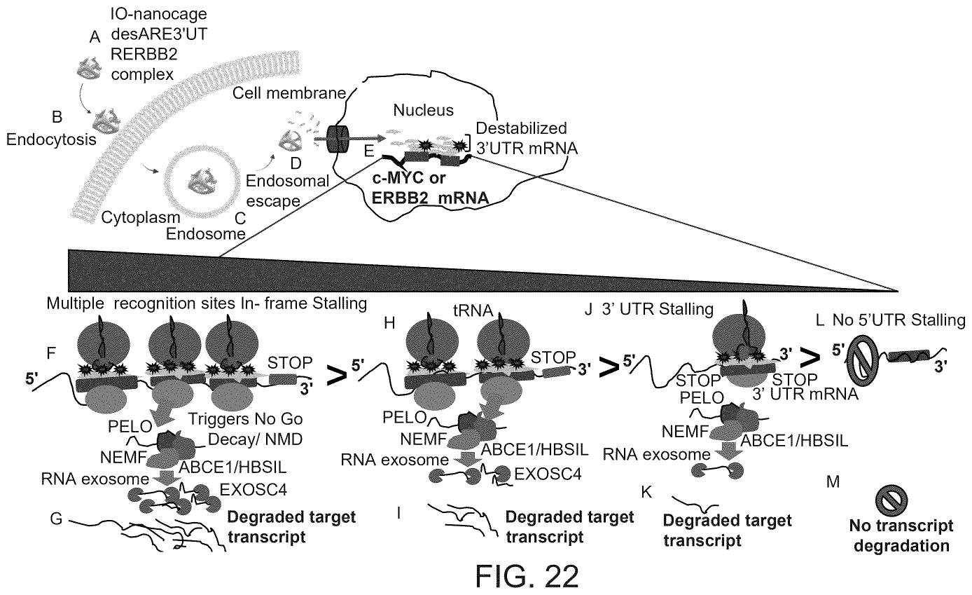

Conceptual framework: To identify the mediators of the ribosome fate switch from the translating to degrading the target transcript once destabilized. We developed a conceptual hypothesis framework and used this to delve into the aspects that were as of yet unknown (FIG. 21). Previous works had shown that the destabilized mRNA is made at such an elevated level (14-17) and that the endogenous target transcript is suppressed. In addition, it had been previously shown that the destabilized 3′UTR mRNA specifically targeted the transcript they were designed for and engaged in their degradation. However, it was unknown as to what encodes or mediate this specificity that we have answered herein. Furthermore, it was not known what proteins mediate the switch of the target transcript from being made to being degraded. This elusive switch molecule is depicted herein as a light switch (FIG. 21). It was unknown also as to what proteins engaged in the degradation of the target transcript. Further to this, the order of the components was unknown, as well as how they carry out their functions to achieve the specific target degradation.

TABLE 1A
primers utilized for validation qPCR

| Gene name | Fwd | Rev | Ontology Theme |
|---|---|---|---|
| SMG9 | CCCCGGTTCTGGATCTGGTA | GTTCTCATGAAGCCACGGGA | Nonsense mediated decay, premature termination codon, in |
| | SEQ ID NO: 95 | SEQ ID NO: 96 | frame stalled ribosome |
| RPL11 | GTGCGGGAGTATGAGTTAAGA | ACCCAGATCGATGTGTTCCTG | ribosome turnover and biogenesis |
| | SEQ ID NO: 97 | SEQ ID NO: 98 | |
| MRT04 | CGAGCTGGTAACAAAGCAGC | CCATACCTCTCTTGAGGGCG | ribosome turnover and biogenesis |
| | SEQ ID NO: 99 | SEQ ID NO: 100 | |
| PELO | AAGTGCCCTTAGAAACCGGG | GCAGGCTTGAGAGTCGAAGT | 3′UTR stalling & degradation |
| | SEQ ID NO: 101 | SEQ ID NO: 102 | |
| ABCE1 | GGTGGTGAACTACAGCGAGT | ACAACTCGAGCTGCCATCAG | 3′UTR stalling & degradation |
| | SEQ ID NO: 103 | SEQ ID NO: 104 | |
| HBS1L | TCCAGTTCCGCCTTCACATC | CACACCTCAAAAAGGACCGC | 3′UTR stalling & degradation |
| | SEQ ID NO: 105 | SEQ ID NO: 106 | |
| ERF1/ETF1 | CCAGGACTGAAAGGCGGTTT | ACCAGATTTCACGAGTGGCA | Premature termination, stalled ribosomes in frame, NMD UPF1 |
| | SEQ ID NO: 107 | SEQ ID NO: 108 | |
| EMG1 | TGGATAGTCCCCTGAACCGA | GCCACAAAAGCGGTCAAAGG | Nacent polypeptide |
| | SEQ ID NO: 109 | SEQ ID NO: 110 | |
| EXOSC4 | TCGGACCAGGGCTACCG | TAGACCACAGCCAGTGCCTT | 3′UTR stalling & degradation |
| | SEQ ID NO: 111 | SEQ ID NO: 112 | |
| RPL3 | CAGTCTTGAAGGTCCGGAGG | CAAGAAGGAGGTGGTGGAGG | Nascent polypeptide |
| | SEO ID NO: 113 | SEQ ID NO: 114 | |
| RPL4 | GCGCAAGTTGGTGTGAACAA | GTCTTTTTCTTTAGGCGTGTGCT | Nascent polypeptide |
| | SEQ ID NO: 115 | SEQ ID NO: 116 | |
| ZNF598 (Hel2) | CCATTAACGACGACGGCAAC | AGCAGCAGTAGCAGCAAGAA | in frame stalling of ribosomes, and senses slow ribosomes and |
| | SEQ ID NO: 117 | SEO ID NO: 118 | di-ribosomes that collides due to slow kinetics |
| NEMF | ACCTTTGGGGTTGAAGGTGA | AGGTGACACGAGCAGTGATG | Nascent polypeptide stalled |
| | SEO ID NO: 119 | SEQ ID NO: 120 | |
| MME1 | AACCATGGCCAACTGTGAGC | CAGACCAACAAGGCGGAATC | RNA binding and Damage |
| | SEO ID NO: 121 | SEQ ID NO: 122 | |
| LSM10 | ACGTCATCTGGGATGTGGAC | AGCTGGTGTGGAAGAATGGC | premRNA DCP binding |
| | SEQ ID NO: 123 | SEQ ID NO: 124 | |
| INTS6 (DDX26 | AAAGGCACACCTGTCAACTG | TTCTCCTTTGCCTGGAAGTGA | 3′ RNA processing, RNA helicase |
| | SEQ ID NO: 125 | SEQ ID NO: 126 | |
| RPL24 | GATAGCTTGTTCTCGTTGAGCC | AAAGAAAAGAACCCGCCGAG | ribosome turnover and biogenesis |
| | SEQ ID NO: 127 | SEQ ID NO: 128 | |
| SKIV2L | AACATGCCTGCTCGTACAGT | TGCCATCTGCACATACTCCC | RNA degradosome 3′UTR stalling & degradation and |
| (SKIC2) | SEQ ID NO: 129 | SEQ ID NO: 130 | PELOTA, HBS1L, ABCE1, RNA alteration |
| RPLS | CATCATGGGCCAGAATGTTGC | TACCATGTCTGGAGTTACGCTG | Nascent polypeptide |
| | SEQ ID NO: 131 | SEQ ID NO: 132 | |
| EXOSCS | GATGTAGGATCCAGCACGAGG | CTGTTGTCTGAATGCCGCCT | RNA exosome exoribonuclease unstable 3′UTR |
| | SEQ ID NO: 133 | SEQ ID NO: 134 | |
| DDX56 | CCTCAGTCTCACAGACCACC | TTCTCCCCCAGGTTACCCTTA | RNA alteration |
| | SEQ ID NO: 135 | SEO ID NO: 136 | |
| NOL6 | ACAGTGAGGGTTTCAGGTGG | GACAAGGACGGGCTGAACC | Nascent premRNA and RNA exosome |
| | SEQ ID NO: 137 | SEQ ID NO: 138 | |

TABLE 1B
sgRNA guides utilized for gene editing

| Name | Sequence | SEQ ID NO: | Modifications |
|---|---|---|---|
| LSM10 guide 1 | CCAGGGCCGGGTAACCACTG | 139 | Yes |
| LSM10 guide 2 | GACATTGTCTATGCGTCCGT. | 140 | Yes |
| RPL11 guide 1 | CTCACCTTTGGAAAACACAG | 141 | Yes |
| RPL11 guide 2 | TGTCCACTGCACACTTCGAG | 142 | Yes |
| PELO guide 1 | GGCTTGAGAGTCGAAGTCGA | 143 | Yes |
| PELO guide 2 | AGAATGACTATGTCAAGATG | 144 | Yes |
| RPL3 guide 1 | CCACCGAGGCCTGCGCAAGG | 145 | Yes |
| RPL3 guide 2 | CCGTGTCATTGCCCACACCC. | 146 | Yes |
| EXOSC4 guide 1 | GCGCAAGCGACGGCCACATG | 147 | Yes |
| EXOSC4 guide 2 | GGCTCGGCCTACATTGAGCA | 148 | Yes |
| Non targeting control | ACAAACGACCUUGAGCAGGG | 149 | Yes |
| NTC1 | | | |

TABLE IC
PELO protein sequences

| | | SEQ |
|---|---|---|
| | | ID |
| Amino acid sequence | | NO: |
| MKLVRKNIEKDNAGQVTLVPEEPEDMWHTYNLVQVGDSLRASTIRKVQTESSTGSVGSNRVRTTLTLCVEAIDFDSQ | wild type | 72 |
| ACQLRVKGINIQENEYVKMGAYHTIELEPNRQFTLAKKQWDSVVLERIEQACDPAWSADVAAVVMQEGLAHICLVTP | | |
| SMILTRAKVEVNIPRKRKGNCSQHDRALERFYEQVVQAIQRHIHFDVVKCILVASPGEVREQFCDYLFQQAVKTDNK | | |
| LLLENRSKFLQVHASSGHKYSLKEALCDPIVASRLSDEKAAGEVKALDDFYKMLQHEPDRAFYGLKQVEKANEAMAI | | |
| DTLLISDELFRHQDVATRSRYVRLVDSVKENAGTVRIFSSLHVSGEQLSQLTGVAAILRFPVPELSDQEGDSSSEED | | |
| MKLVRKNIEKDNAGQVTLVPEEPEDMWHTYNLVQVGDSLRASTIRKVQTESSTGSVGSNRVRTTLELCVEAIDFDSQ | truncated | 73 |
| ACQLRVKGINIQENEYVKMGAYHTIELEPNRQFTLAKKQWDSVVLERIEQACDPAWSADVAAVVMQEGLAHICLVTP | | |
| SMILTRAKVEVNIPRKRKGNCSQHDRALERFYEQVVQAIQRHIHFDVVKCILVASPGEVREQFCDYMFQQAVKTDNK | | |
| LLLENRSKELQVHASSGHKYSLKEALCDPIVASRLSDEKAAGEVKALDDFYKMLQHEPDRAFYGEKQVEKANEAMAI | | |
| DTLLISDELFRHQDVATRSRYVRL | | |
| NKLVRKNIEKDNAGQVTLVPEEPEDMWHTYNLVQVGDSLRASTIRKVQTESSTGSVGSNRVATTLELCVEAIDFDSQ | truncated | 74 |
| ACQLRVKGINIQENEYVKMGAYHTIELEPNRQFTLAKKQWDSVVLERIEQACDPAWSADVAAVVMQEGLAHICLVTP | | |
| SMILTRARVEVNIPRKREGNCSQHDRALERFYEQVVQAIQRHIHEDVVKCILVASPGFVREQFCDYMFQQAVKTQNK | | |
| LLLENRSKFLQVHASSGHKYSLKEALCDPIVASRISDEKAAGEVKALDDFYKMLQHEPDRAFYGLKQVEKANEAMAI | | |
| DTLLISDELFRHQDVATRSRYVRLVDSYKENAGTVRIFSSLHVSG | | |
| MKLVRKNIEKDNAGQVTLVPEEPEDMWHTYNLVQVGDSLRASTIRKVQTESSTGSVGSNRVRITLTLCVEAIDFDSQ | truncated | 75 |
| ACQLRVKGINIQENEYVEMGAYHTIELEPNRQFTLAKKQWDSVVLERIEQACDPAWSADVAAVVMQEGLAHICLVTP | | |
| SMILTRAKVEVNIPRKAKGNCSQHDRALERFYEQVVQAIQRHIHFDVVKCILVASPGEVREQFCDYMFQQAVKTDNK | | |
| LLLENRSKELQVHASSGHKYSLREALCQPYVASRLSDTKAAGEVKALDDFYKMLQHEPDRAFYGLKQVEKANEAMAI | | |
| DTLLISDELFREQDVATRSRYVRLVDSVKENAGTVRIFSSLHVSGEQLSQLIGVAAILRFPVPELS | | |
| MKLVRKNIEKDNAGQVTLVPEEPEDMWHTYNLVQVGDSLRASTIRKVQTESSTGSVGSNRVRTTLTLCVEAIDFDSQ | fusion to | 76 |
| ACQLRVKGINIQENEYVEMGAYHTIELEPNRQFTLAKKQWDSVVLERIEQACDPAWSADVAAVVMQEGLAHICLVTP | His tag | |
| SMILTRAKVEVNIPRKRKGNCSQHDRALERFYEQVVQAIQRHIHFDVVKCILVASPGEVREQFCDYMFQQAVKTDNK | | |
| LLLENRSKFLQVHASSGHKYSLKEALCDPIVASRLSDTKAAGEVKALDDFYKMLQHEPDRAFYGLKQVEKANEAMAI | | |
| DTLLISDELFRHQDVAIRSRYVRLVDSVKENAGTVRIFSSLHVSGEQLSQLTGVAAILRFPVPELSDQEGDSSSEED | | |
| TRTRPLEQKL | | |
| MKLVRKNIEKDNAGQVILVPEEPEDMWHTYNLVQVGDSLRASTIRKVQTESSTGSVGSNRVRTTLTLCVEAIDFDSQ | fusion to | 77 |
| ACQLRVKGTNIQENEYVENGAYHTIELEPNRQFTLAKKQWDSVVLERIEQACDPAWSADVAAVVMQEGLAHICLVEP | His tag | |
| SMILERAKVEVNIPRKRKGNCSQHDRALERFYEQVVQAIQRHIHFDVVKCILVASPGEVREQFCDYMFQQAVATDNK | | |
| LLLENRSKELQVHASSGHKYSLKEALCDPTVASRLSDTKAAGEVKALDDFYKMLCHEPDRAFYGLKQVEKANEAMAI | | |
| DTLLISDELFRHQDVATRSRYVRLVDSVKENAGTVRIESSLAVSGEQLSQLTGVAAILRFPVPELSDQEGDSSSEED | | |
| TRTRPLEQKLISEEDLAANDILDYKDDDDKV | | |
| NKLVRKNIEKDNAGQVTLVPEEPEDMWHTYNLVQVGDSLRASTIRKVQTESSTGSVGSNRVRITLTLCVEAIDFDSQ | fusion to | 78 |
| ACQLRVKGINIQENEYVEMGAYHTIELEPNRQFTLAKKQWDSVYLERIEQACDPAWSADVAAVVMQEGLAHICLVEP | His tag | |
| SMILTRAKVEVNIPRKRKGNCSQHDRALERFYEQVVQAIQRHIHFDVVKCILVASPGFVREQFCDYMFQCAVKTDNK | | |
| LLLENRSKFLQVHASSGHKYSLKEALCDPIVASRLSDTKAAGEVKALDDFYKMLQHEPDRAFYGEKQVEKANEAMAI | | |
| DTLLISDELFRHQDVAIRSRYVRLVDSVKENAGIVRIFSSLHVSGEQLSQLTGVAAILRFPVPELSDQEGDSSSEED | | |
| TRTRPLEQKLISEEDLAANDILDYKDDDDKV | | |

TABLE ID
EXOSC4 protein sequences

| | | SEQ |
|---|---|---|
| | | ID |
| Amino acid sequence | | NO: |
| MAGLELLSDQGYRVDGRRAGELRKIQARMGVFAQADGSAYIEQGNIKALAVVYGPHEIRGSRARALPDRALVNCQYS | wild type | 79 |
| SATFSIGERKRRPHGDRKSCEMGLQLRQTFEAAILTQLHPRSQIDIYVQVLQADGGTYAACVNAATLAVLDAGIPMR | | |
| DEVCACSAGFVDGTALADLSHVEEAAGGPQLALALLPASGQIALLEMDARLHEDHLERVLEAAAGAARDVHTLLDRV | | |
| VRQHVREASILLGD | | |
| MAGLELLSDQGYRVDGRRAGELRKIQARMGVFAQADGSAYIEQGNTKALAVVYGPHEIRGSRARALPDRALVNCQYS | truncated | 80 |
| SATFSTGERKRRPHGDRKSCEMGLQLRQTFEAAILTQLHPRSQIDIYVQVLQADGGTYAACVNAATLAVIDAGIPMR | | |
| DEVCACSAGFVDGTALADLSHVEEAAGGPQLALALLPASGQIALLEMDARLHEDHLERVLEAAAQAA | | |
| MAGLELLSDQGYRVDGRRAGELRKIQARMGVFAQADGSAYIEQGNIKALAVVYGPHEIRGSRARALPDRALVNCQYS | truncated | 81 |
| SATFSTGERKRRPHGDRKSCEMGLQLRQTFEAAILTQLHPRSQIDIYVQVLQADGGTYAACVNAATLAVLDAGIPMR | | |
| DEVCACSAGFVDGTALADLSHVEEAAGGPQLALALLPASGQIALLEMDARLHEDHLERVLEAAAQAARDVHTLLDRY | | |
| VRQH | | |
| MAGLELLSDQGYRVDGRRAGELRKIQARMGYFAQADGSAYIEQGNTKALAVVYGPHEIRGSRARALPQRALVNCQYS | fusion to | 82 |
| SAIFSTGERKRRPHGDRKSCEMGLQLRQTFEAAILTQLHPRSQIDIYVQVLQADGGTYAACVNAATLAVIDAGIPMR | His tag | |
| DFVCACSAGFVDGTALADLSHVEEAAGGPQLALALLPASGQIALLEMDARLHEDHLERVLEAAAGAARDVHTLLDRY | | |
| VRQHVREASILLGDTRT | | |
| MAGLELLSDQGYRVDGRRAGELRKIQARMGVFAQADGSAYIEQGNTKALAVVYGPHEIRGSRARALPDRALVNCQYS | fusion to | 83 |
| SAIFSTGERKRRPHGDRKSCEMGLQLRQTFEAAILIQLHPRSQIDIYVQVLQADGGTYAACVNAATLAVIDAGIPMR | His tag | |
| DFVCACSAGFVDGTALADLSHVEEAAGGPQLALALLPASGQIALLEMDARLHEDHLERVLEAAAGAARDVHTLLDRV | | |
| VRQHVREASILLGDTRTRPLEQKLISEEDLA | | |
| MAGLELLSDOGYRVDGRRAGELRKIQARMGVFAQADGSAYIEQGNTKALAVVYGPHEIRGSRARALPDRALVNCQYS | fusion to | 84 |
| SAIFSTGERKRRPHGDRKSCEMGLQLRQTFEAAILTQLHPRSQIDIYVQVLQADGGTYAACVNAATLAVLDAGIPMR | His tag | |
| DFVCACSAGFVDGTALADLSHVEEAAGGPQLALALLPASGQIALLEMDARLHEDHLERVLEAAAGAARDVHTLLDRY | | |
| VRQHVREASILLGDTRTRPLEQKLISEEDLAANDILDYKDDDDKV | | |

TABLE 1E
RPL3 protein sequences

| | | SEQ |
|---|---|---|
| | | ID |
| Amino acid sequence | | NO: |
| MSHRKESAPRHGSLGFLPRKRSSRHRGKVKSFPKDDPSKPVHLTAFLGYKAGMTHIVREVDRPGSKVNKKEVVEAVT | wild type | 85 |
| IVETPPMVVVGIVGYVETPRGERTFKTVFAEHISDECKRRFYKNWHKSKKKAHLMEIQVNGGTVAEKLDWARERLEQ | | |
| QVPVNQVFGQDEMIDVIGVIKGKGYKGVTSRWHTKKLPRKTHRGERKVACIGAWHPARVAFSVARAGQKGYHHRTEI | | |
| NKKIYKIGQGYLIKDGKLIKNNASTDYDLSDKSINPLGGFVHYGEVINDFVMLKGCVVGTKKRVLTERKSLLVQTER | | |
| RALEKIDIKFIDETSKEGHGRFQTMEEKKAFMGPLKKDRIAKEEGA | | |
| MSHRKESAPRHGSLGFLPRKRSSRHRGKVKSFPKDDPSKPVHLTAFLGYKAGMTHIVREVDRPGSKVNKKEVVEAVT | truncated | 86 |
| IVETPPMVVVGIVGYVETPRGERTEKTVFAERISDECKRRFYRNWHKSKKKAHLMEIQVNGGIVAEKLDWARERLEQ | | |
| QVPVNQVEGQDEMIDVIGVIKGKGYKGVTSRWHTKKLPRKTHRGERKVACIGAWHPARVAFSVARAGQKGYHHRTEI | | |
| NKKIYKIGQGYLIKDGKLIKNNASTDYDLSDKSINPLGGFVHYGEVINDFVMLKGCVVGTKKRVLTLRKSLLVQTKR | | |
| MSHRKESAPRHGSLGFLPRKRSSRHRGKVKSFPKDDPSKPVHITAFLGYKAGMTHIVREVDRPGSKVNKKEVVEAVT | truncated | 87 |
| IVETPPMVVVGIVGYVETPRGLRTFKTVFAEKISDECKRRFYKNWHKSKKKAHLMEIQVNGGIVAEKLDWARERLEQ | | |
| QVPVNQVFGQDEMIDVIGVTKGKGYKGVTSRWHTKKLPRKTHRGERKVACIGAWHPARVAFSVARAGQKGYHHRTEI | | |
| NKKIYKIGQGYLIKQGKLIKNNASTDYDLSDKSINPLGGFVHYGEVINDFVMLKGCVVGIKKRVLTLRKSLLVQTKR | | |
| RALEKIDLKFIDETSKFGH | | |
| MSHRKFSAPRHGSLGELPRKRSSRHRGKVKSFPKDDPSKPVALTAFLGYKAGMTHIVREVDRPGSKVNKKEVVEAVT | truncated | 88 |
| IVETPPMVVVGIVGYVETPRGERTFKTVFAEHISDECKERFYKNWHKSKKKAHLMEIQVNGGTVAEKLDWARERLEQ | | |
| QVPVNQVEGQDEMIDVIGVIKGKGYKGVTSRWHTKKLPRKTHRGLRKVACIGAWHPARVAFSVARAGQKGYHHRIEI | | |
| NKKIYKIGQGYLIKDGKLIKNNASTDYDLSDKSINPLGGFVHYGEVTNDFVMLKGCVVGTKKRVLELRKSLLVQTKR | | |
| RALEKIDLKFIDITSKFGHGRFQTMEEKKAFMGPLKKD | | |
| MSHRKFSAPRHGSIGELPRKRSSRHRGKVKSFPKDDPSKPVHLTAFLGYKAGMTHIVREVDRPGSKVNKKEVVEAVT | fusion to | 89 |
| IVETPPMVVVGIVGYVETPRGERTFKTVFAEHISDECKRRFYKNWHKSKKKAHLMEIQVNGGTVAEKLDWARERLEQ | His tag | |
| QVPVNQVEGQDEMIDVIGVIKGKGYKGVTSRWHTKKLPRKTHRGERKVACIGAWHPARVAFSVARAGQKGYHHRTEI | | |
| NKKIYKIGQGYLIKDGKLIKNNASTDYDLSDKSINPLGGFVHYGEVINDFVMLKGCVVGTKKRVLTLRKSLLVQTKR | | |
| RALEKIDLKFIDITSKFGHGRFQTMEEKKAFMGPLKKDRIAKEEGATRTRPLEQKLIS | | |
| MSHRAFSAPRHGSLGFLPRKRSSRHRGKVKSFPKDDPSKPVHLTAFLGYKAGMTHIVREVDRPGSKVNKKEVVEAVT | fusion to | 90 |
| IVETPPMVVVGIVGYVETPRGLRTFKIVFAEHISDECKRRFYKNWHKSKKKAHLMEIQVNGGEVAEKLDWARERLEQ | His tag | |
| QVPVNQVEGQDEMIDVIGVTKGKGYKGVTSRWHTKKLPRKTHRGLRKVACIGAWHPARVAFSVARAGQKGYHERTEI | | |
| NKKIYKIGQGYLIKDGKLIKNNASTDYDLSDKSINPLGGFVHYGEVINDFVMLKGCVVGIKKRVLELRKSLLVQTKR | | |
| RALEKIDLKFIDETSKEGHGRFQTMEEKKAFMGPIKKDRIAKEEGATRTRPLEQKLISEEDLAANDILDYKDDDDKV | | |

TABLE 1F
Recognition sequences on target c-MYC mRNA

| | Position on | SEQ |
|---|---|---|
| Recognition sequence | target mRNA | ID NO: |
| GAGAUGGUGACCGAGCUGCUGGGAGGAGACAUGGUGAACCAGAGUUUCAUCUGCGACCCGGAC | Exon 2 | 91 |
| GACGAGACCUUCAUCAAAAACAUCAUCAUCCAGGACUGUAUGUGGAGCGGCUUCUCGGCCGCC | | |
| GCCAAG | | |
| CUGGAUUUUUUUCGGGUAGUGGAAAACCAGGUAAGCACCGAAGUCCACUUGCCUUUUAAUUUA | Exons 1-3 and | 92 |
| UUUUUUUAUCACUUUAAUGCUGAGAUGAGUCGAAUGCCUAAAUAGGGUGUCUUUUCUCCCAUU | introns 1-2 | |
| CCUGCGCUAUUGACACUUUUCUCAGAGUAGUUAUGGUAACUGGGGCUGGGGUGGGGGGUAAUC | | |
| CAGAACUGGAUCGGGGUAAAGUGACUUGUCAAGAUGGGAGAGGAGAAGGCAGAGGGAAAACGG | | |
| GAAUGGUUUUUAAGACUACCCUUUCGAGAUUUCUGCCUUAUGAAUAUAUUCACGCUGACUCCC | | |
| GGCCGGUCGGACAUUCCUGCUUUAUUGUGUUAAUUGCUCUCUGGGUUUUGGGGGGCUGGGGGU | | |
| UGCUUUGCGGUGGGCAGAAAGCCCCUUGCAUCCUGAGCUCCUUGGAGUAGGGACCGCAUAUCG | | |
| CCUGUGUGAGCCAGAUCGCUCCGCAGCCGCUGACUUGUCCCCGUCUCCGGGAGGGCAUUUAAA | | |
| UUUCGGCUCACCGCAUUUCUGACAGCCGGAGACGGACACUGCGGCGCGUCCCGCCCGCCUGUC | | |
| CCCGCGGCGAUUCCAACCCGCCCUGAUCCUUUUAAGAAGUUGGCAUUUGGCUUUUUAAAAAGC | | |
| AAUAAUACAAUUUAAAACCUGGGUCUCUAGAGGUGUUAGGACGUGGUGUUGGGUAGGCGCAGG | | |
| CAGGGGAAAAGGGAGGCGAGGAUGUGUCCGAUUCUCCUGGAAUCGUUGACUUGGAAAAACCAG | | |
| GGCGAAUCUCCGCACCCAGCCCUGACUCCCCUGCCGCGGCCGCCCUCGGGUGUCCUCGCGCCC | | |
| GAGAUGCGGAGGAACUGCGAGGAGCGGGGCUCUGGGGGGUUCCAGAACAGCUGCUACCCUUGG | | |
| UGGGGUGGCUCCGGGGGAGGUAUCGCAGCGGGGUCUCUGGCGCAGUUGCAUCUCCGUAUUGAG | | |
| UGCGAAGGGAGGUGCCCCUAUUAUUAUUUGACACCCCCCUUGUAUUUAUGGAGGGGUGUUAAA | | |
| GCCCGCGGCUGAGCUCGCCACUCCAGCCGGCGAGAGAAAGAAGAAAAGCUGGCAAAAGGAGUG | | |
| UUGGACGGGGGCGGUACUGGGGGUGGGGACGGGGGGGGUGGAGAGGGAAGGUUGGGAGGGGCU | | |
| GCGGUGCCGGGGGGGGUAGGAGAGCGGCUAGGGCGCGAGUGGGAACAGCCGCAGCGGAGGGGC | | |
| CCCGGCGCGGAGCGGGGUUCACGCAGCCGCUAGCGCCCAGGCGCCUCUCGCCUUCUCCUUCAG | | |
| GUGGCGCAAAACUUUGUGCCUUGGAUUUUGGCAAAUUGUUUUCCUCACCGCCACCUCCCGCGG | | |
| CUUCUUAAGGGCGCCAGGGCCGAUUUCGAUUCCUCUGCCGCUGGGGGGCCGACUCCCGGGCUU | | |
| UGCGCUCCGGGCUCCCGGGGGAGCGGGGGCUCGGCGGGCACCAAGCCGCUGGUUCACUAAGUG | | |
| CGUCUCCGAGAUAGCAGGGGACUGUCCAAAGGGGGUGAAAGGGUGCUCCCUUUAUUCCCCCAC | | |
| CAAGACCACCCAGCCGCUUUAGGGGAUAGCUCUGCAAGGGGAGAGGUUCGGGACUGUGGCGCG | | |
| CACUGCGCGCUGCGCCAGGUUUCCGCACCAAGACCCCUUUAACUCAAGACUGCCUCCCGCUUU | | |
| GUGUGCCCCGCUCCAGCAGCCUCCCGCGACGAUGCCCCUCAACGUUAGCUUCACCAACAGGAA | | |
| CUAUGACCUCGACUACGACUCGGUCCAGCCGUAUUUCUACUGCGACGAGGAGGAGAACUUCUA | | |
| CCAGCAGCAGCAGCAGAGCGAGCUGCAGCCCCCGGCGCCCAGCGAGGAUAUCUGGAAGAAAUU | | |
| CGAGCUGCUGCCCACCCCGCCCCUGUCCCCUAGCCGCCGCUCCGGGCUCUGCUCGCCCUCCUA | | |
| CGUUGCGGUCACACCCUUCUCCCUUCGGGGAGACAACGACGGCGGUGGCGGGAGCUUCUCCAC | | |
| GGCCGACCAGCUGGAGAUGGUGACCGAGCUGCUGGGAGGAGACAUGGUGAACCAGAGUUUCAU | | |
| CUGCGACCCGGACGACGAGACCUUCAUCAAAAACAUCAUCAUCCAGGACUGUAUGUGGAGCGG | | |
| CUUCUCGGCCGCCGCCAAGCUCGUCUCAGAGAAGCUGGCCUCCUACCAGGCUGCGCGCAAAGA | | |
| CAGCGGCAGCCCGAACCCCGCCCGCGGCCACAGCGUCUGCUCCACCUCCAGCUUGUACCUGCA | | |
| GGAUCUGAGCGCCGCCGCCUCAGAGUGCAUCGACCCCUCGGUGGUCUUCCCCUACCCUCUCAA | | |
| CGACAGCAGCUCGCCCAAGUCCUGCGCCUCGCAAGACUCCAGCGCCUUCUCUCCGUCCUCGGA | | |
| UUCUCUGCUCUCCUCGACGGAGUCCUCCCCGCAGGGCAGCCCCGAGCCCCUGGUGCUCCAUGA | | |
| GGAGACACCGCCCACCACCAGCAGCGACUCUGGUAAGCGAAGCCCGCCCAGGCCUGUCAAAAG | | |
| UGGGCGGCUGGAUACCUUUCCCAUUUUCAUUGGCAGCUUAUUUAACGGGCCACUCUUAUUAGG | | |
| AAGGAGAGAUAGCAGAUCUGGAGAGAUUUGGGAGCUCAUCACCUCUGAAACCUUGGGCUUUAG | | |
| CGUUUCCUCCCAUCCCUUCCCCUUAGACUGCCCAUGUUUGCAGCCCCCCUCCCCGUUUGUCUC | | |
| CCACCCCUCAGGAAUUUCAUUUAGGUUUUUAAACCUUCUGGCUUAUCUUACAACUCAAUCCAC | | |
| UUCUUCUUACCUCCCGUUAACAUUUUAAUUGCCCUGGGGGGGGGUGGCAGGGAGUGUAUGAAU | | |
| GAGGAUAAGAGAGGAUUGAUCUCUGAGAGUGAAUGAAUUGCUUCCCUCUUAACUUCCGAGAAG | | |
| UGGUGGGAUUUAAUGAACUAUCUACAAAAAUGAGGGGCUGUGUUUAGAGGCUAGGCAGGGCCU | | |
| GCCUGAGUGCGGGAGCCAGUGAACUGCCUCAAGAGUGGGUGGGCUGAGGAGCUGGGAUCUUCU | | |
| CAGCCUAUUUUGAACACUGAAAAGCAAAUCCUUGCCAAAGUUGGACUUUUUUUUUUCUUUUAU | | |
| UCCUUCCCCCGCCCUCUUGGACUUUUGGCAAAACUGCAAUUUUUUUUUUUUUAUUUUUCAUUU | | |
| CCAGUAAAAUAGGGAGUUGCUAAAGUCAUACCAAGCAAUUUGCAGCUAUCAUUUGCAACACCU | | |
| GAAGUGUUCUUGGUAAAGUCCCUCAAAAAUAGGAGGUGCUUGGGAAUGUGCUUUGCUUUGGGU | | |
| GUGUCCAAAGCCUCAUUAAGUCUUAGGUAAGAAUUGGCAUCAAUGUCCUAUCCUGGGAAGUUG | | |
| CACUUUUCUUGUCCAUGCCAUAACCCAGCUGUCUUUCCCUUUAUGAGACUCUUACCUUCAUGG | | |
| UGAGAGGAGUAAGGGUGGCUGGCUAGAUUGGUUCUUUUUUUUUUUUUUUCCUUUUUUAAGACG | | |
| GAGUCUCACUCUGUCACUAGGCUGGAGUGCAGUGGCGCAAUCAACCUCCAACCCCCUGGUUCA | | |
| AGAGAUUCUCCUGCCUCAGCCUCCCAAGUAGCUGGGACUACAGGUGCACACCACCAUGCCAGG | | |
| CUAAUUUUUGUAAUUUUAGUAGAGAUGGGGUUUCAUCGUGUUGGCCAGGAUGGUCUCUCCUGA | | |
| CCUCACGAUCCCCCCACCUCGGCCUCCCAAAGUGCUGGGAUUACAGGUGUGAGCCAGGGCACC | | |
| AGGCUUAGAUGUGGCUCUUUGGGGAGAUAAUUUUGUCCAGAGACCUUUCUAACGUAUUCAUGC | | |
| CUUGUAUUUGUACAGCAUUAAUCUGGUAAUUGAUUAUUUUAAUGUAACCUUGCUAAAGGAGUG | | |
| AUUUCUAUUUCCUUUCUUAAAGAGGAGGAACAAGAAGAUGAGGAAGAAAUCGAUGUUGUUUCU | | |
| GUGGAAAAGAGGCAGGCUCCUGGCAAAAGGUCAGAGUCUGGAUCACCUUCUGCUGGAGGCCAC | | |
| AGCAAACCUCCUCACAGCCCACUGGUCCUCAAGAGGUGCCACGUCUCCACACAUCAGCACAAC | | |
| UACGCAGCGCCUCCCUCCACUCGGAAGGACUAUCCUGCUGCCAAGAGGGUCAAGUUGGACAGU | | |
| GUCAGAGUCCUGAGACAGAUCAGCAACAACCGAAAAUGCACCAGCCCCAGGUCCUCGGACACC | | |
| GAGGAGAAUGUCAAGAGGCGAACACACAACGUCUUGGAGCGCCAGAGGAGGAACGAGCUAAAA | | |
| CGGAGCUUUUUUGCCCUGCGUGACCAGAUCCCGGAGUUGGAAAACAAUGAAAAGGCCCCCAAG | | |
| GUAGUUAUCCUUAAAAAAGCCACAGCAUACAUCCUGUCCGUCCAAGCAGAGGAGCAAAAGCUC | | |
| AUUUCUGAAGAGGACUUGUUGCGGAAACGACGAGAACAGUUGAAACACAAACUUGAACAGCUA | | |
| CGGAACUCUUGUGCGUAA | | |
| CACGUG | MYC E-box | |
| | element | |
| AACGUG | MYC E-box | |
| | element | |

TABLE 1G
Recognition sequences on target ERBB2 mRNA

| | Position | SEQ |
|---|---|---|
| Recognition sequence | on target | ID NO: |
| AGUGUGCACCGGCACAGACAUGAAGCUGCGGCUCCCUGCCAGUCCCGAGACCCACCUGGACAU | Exons 2-7, | 93 |
| GCUCCGCCACCUCUACCAGGGCUGCCAGGUGGUGCAGGGAAACCUGGAACUCACCUACCUGCC | introns 2-6 | |
| CACCAAUGCCAGCCUGUCCUUCCUGCAGGUGAGGCCCGUGGGCAACCCAGCCAGGCCCUGCCU | | |
| CCAGCUGGGCUGAGCCCUCUGUUUACAGGUGGGUGGCAGAAGAAGGUGCCCUGCCCUUCUGUU | | |
| UCCUCUCUUGUUGUGGUUUCUCAACCAGGAAGUCCUUUCUAACAUCUAACCCCCAUUCAUUUU | | |
| ACUGCAGAAUCAGUUGACUCUCUCUAUAACGUGGCUGGCCGAGGUCAUGUCUGGAUGGGAUGC | | |
| GUCUGUGUUUCCGCUAAAUCUUGUGCUCUCUUGCCAGCAUGAUCAUGUCCCCUGUCCACCUGC | | |
| UCCAGCCACUAUCCCUCUCCCACUUACAGCAGAAGAAAGGGCUGGUGAGAAAGGUGGAUUACA | | |
| GGCCCACUUCUGCCACUGACGAGCCCUAUGAAUGUGGCCUACACCCCCUUAGCUUCACUGGGU | | |
| CUCAGUUUCCCUAUCUGUAUAUUGGGAGCAGUUGUGAAGCUCAGAAGAGAAAUGUCUGUGAAA | | |
| AGGUUAUGAACAGGAGGGAGAGUGGAAACCAACCUGCUGGAUCGUGUCCACAGACCCUGGAAU | | |
| GGGGCCACAUGCUUGGUUUGUCAAAUUGCAGACGCCGGCCGGGUGCGAUGGCUCAUGCCUGUA | | |
| AUCCCAGCACUUUGGGAGGCCGAGGCGGACAGAUCACUUGAGGUCGGGAGUUCGAGACCAGCC | | |
| UGACCAACAUGGAGAAACCCCGUCUCUACUGAAAAUACAAAAUUAGCCAGGCAUGGUGGCACA | | |
| UGCCUAUAAUCCCAGCUACUUGGGAAGGCUGAGGCAGGAGAAUCACUUGAACCUGGGAGACGG | | |
| AGGUUGUGGUGAGCCUAGAUCGUGCCAUUGUACUCCAGCCUGGGCAACAAGAGUGAAACUCCG | | |
| UCUCAAAAAAAAAAAAUUUGCAGACGCCAUCCCAUCCAGGCCUUUGCUUUCACUGAUGAAGAA | | |
| ACUGAGAUACAGAGAGGGCAGGGCACCUGUUCGGAGUUUAUGAAAUGCCCCCCCACCAUUAUC | | |
| UUUCUUGAUCAUAUAAGAAUCUGGUGAGGCAAGGUAGGGCGUGAUCUUUAUCUCUAUUUUAUC | | |
| GUUUUAUUUAAGCGGGAACAGGACUGCUCAGUGGCUGGGGGCCUUGCCCAAGAUCUCCAAGUA | | |
| CUGGGGAACCCCAGGGAGGCCCUGGGGGGUGGCAGUGUUCCUAUUUCAGCCCCACUCUGCUUC | | |
| CCCCUCCCAGGAUAUCCAGGAGGUGCAGGGCUACGUGCUCAUCGCUCACAACCAAGUGAGGCA | | |
| GGUCCCACUGCAGAGGCUGCGGAUUGUGCGAGGCACCCAGCUCUUUGAGGACAACUAUGCCCU | | |
| GGCCGUGCUAGACAAUGGAGACCCGCUGAACAAUACCACCCCUGUCACAGGGGCCUCCCCAGG | | |
| AGGCCUGCGGGAGCUGCAGCUUCGAAGCCUCACAGGUGGCCUUCACCGUCAUUGAAACCUUCU | | |
| CUUGGUUAUUCAGAGCUGACCAGGGCCACUGCUAACCAGGGGGAGGCUUUGUGUGCAUUAGAA | | |
| AUGGUGUCCCUUCUGGGCAGACGCAGGCAGAGCCCGGGAAGACGCCCUCAGAAGAUUGGAAAA | | |
| AGAUUCCCCUUCUUCCUGGGAAGUUGUAGCUUGCGUCAGCACAUAUAAUUCAAUCGUGAGAAU | | |
| GCAGGCUGGGUUUUUGCCCCCACUUGGCUGAGUGAAGUGUACAGUGAACAACCUAUGUAACUA | | |
| UUUGCUGGCCCUGGAGCCGACUCUGCCCCAGAGUCUGGGUGCCAGGUGCUUUGCCCGCAUGGC | | |
| CCAUUUCAGUCACGCUGCAGUCCUGUCAGGAAAAAAUCAGUGUUAUUCUCAUUCUACAUAUGA | | |
| GAAAACUGAGGCUUGCAGAUAUAAGGGCCAAAAGUUACACAGCUAGUGAGUGAUGGGGCUGAG | | |
| UUUCAGACUCCACAGUCUCUUAACCACCAAGCAGCAUGCCCAGAGUAGAGGUGAGAAGGAAGG | | |
| AGAGAGCUGCGGUCCACAUGAGCAUCUGGACCUAGCAUGGACAACUCACUCCUCCCUGGCUCU | | |
| CGCUUUGUUCUUGUUGCGGGUGUGGUGGUGGUGGGACUCAAAGACGGUAAAGAUAGCUUUCUC | | |
| UCCUCCCUGGGGAAUCUGGGGGUUGUUUAAAAGGCCUGCUCCUCUUUUAGAAGGCAGGAGGGC | | |
| CCCAAGGGAAGCAGAAGGUGACAGAAGGGGAAAGGGUCCUCUGAUCAUUGCUCACCCCACAGA | | |
| GAUCUUGAAAGGAGGGGUCUUGAUCCAGCGGAACCCCCAGCUCUGCUACCAGGACACGAUUUU | | |
| GUGGAAGGACAUCUUCCACAAGAACAACCAGCUGGCUCUCACACUGAUAGACACCAACCGCUC | | |
| UCGGGCCUGUAAGCCAUGCCCCUCCCUGCUGCCUCUUCUCUCAGACAGCCUGACCCCAGCCGC | | |
| AAACUCCCAACUUACAACCCAGUGCCUGCCCGCCACUGCCCCAGCCGCCUACACCACCCAUUU | | |
| CCUCCCUCUCUGUCCCUCCUGCCAUCUCCCUGUGCCUCUUCAUCUCUGGGGUUCUCUGUCUUG | | |
| UCUCCCUCUGCUUAUAGGUUGUGCCUCUGGUUUGGGGGCCUCUCAGCCUGUCUGGGUCCCUCC | | |
| CUUGCUGUGCAGUUGGCCUCGUGGCCUCUGCUGCUGUUUGUGCCUCUCUCUGUUACUAACCCG | | |
| UCCUCUCGCUGUUAGACAUCUCUCUCACUGCCUGUCUCUGGUUCUGUCCUCAGGCCACCCCUG | | |
| UUCUCCGAUGUGUAAGGGCUCCCGCUGCUGGGGAGAGAGUUCUGAGGAUUGUCAGAGCCGUGA | | |
| GUCUCAGGGAGGCCUGGAGUCAGGGAAGGGGAGGGCUGGGGCCGGGUGGAAUGCAGGUGUCAU | | |
| ACAGGUGACAUGGGAGGGGUGGGAUAACAGGCUUGGGAUGUCUCCCCUGGGCCAGGUAGUCUC | | |
| CCUAGAAGGUGAUGCUGAUGAGGGUCUGGUGCCCAGGGCGCCACUCAGCCCUCAUCCUGCCCU | | |
| UUGCCCAACAGUGACGCGCACUGUCUGUGCCGGUGGCUGUGCCCGCUGCAAGGGGCCACUGCC | | |
| CACUGACUGCUGCCAUGAGCAGUGUGCUGCCGGCUGCACGGGCCCCAAGCACUCUGACUGCCU | | |
| GGUAUGUGCCUCUGCUUUGUGCCCAAUGUGCUCUACCCCCCAGGAUGCAAGGGGUGGGCACCC | | |
| UGCCUGGUACUGCCCUAUUGCCCCUGGCACACCAGGGCAAAACAGCACAGUGAAAGCCAGCCA | | |
| CCUGUCCCCCCAGGCCUGCCUCCACUUCAACCACAGUGGCAUCUGUGAGCUGCACUGCCCAGC | | |
| CCUGGUCACCUACAACACAGACA | | |
| GCCCCAUCCCUGCCUGUGGCUAAGAGCACCCUCCUGCAGAGGGUGGGAAGGAGAGAUGAGUCC | Exons 26-27, | 94 |
| AGUAUGCCAGGCCCCUCACGGAAGGCUGCAUGCUGGGCUGGGGAGGGGCCACCAUCCUGCCUC | intron 26 | |
| UCCUUCCUCCACAGAAUGAGGACUUGGGCCCAGCCAGUCCCUUGGACAGCACCUUCUACCGCU | | |
| CACUGCUGGAGGACGAUGACAUGGGGGACCUGGUGGAUGCUGAGGAGUAUCUGGUACCCCAGC | | |
| AGGGCUUCUUCUGUCCAGACCCUGCCCCGGGCGCUGGGGGCAUGGUCCACCACAGGCACCGCA | | |
| GCUCAUCUACCAGGGUCAGUGCCCUCGGUCACACUGUGUGGCUGUCUGCUUACCUCCCCCAAC | | |
| CCCGGUGGACUAGGGUCCCUUUCUCUGAUGUUCCCUCAACUGUCACCUCUCAAGGAAACCCCA | | |
| UUAUCCCUACAAAAAAUUCUUACUGCCUUCCAACCCCUGUGACCCCAUUCUCUCCACGGUGAC | | |
| UGUGUCAUACCCCAAAGGUGACCUCUGUUUUUCUCCUGUGACCCUGUCACCCUCCAUGGAGUC | | |
| CCCAUCCCAGAUCCGUGAGUGACCCCCAUCAUGACUUUCUUUCUUGUCCCCAGAGUGGCGGUG | | |
| GGGACCUGACACUAGGGCUGGAGCCCUCUGAAGAGGAGGCCCCCAGGUCUCCACUGGCACCCU | | |
| CCGAAGGGGCUGGCUCCGAUGUAUUUGAUGGUGACCUGGGAAUGGGGGCAGCCAAGGGGCUGC | | |
| AAAGCCUCCCCACACAUGACCCCAGCCCUCUACAGCGGUACAGUGAGGACCCCACAGUACCCC | | |
| UGCCCUCUGAGACUGAUGGCUACGUUGCCCCCCUGACCUGCAGCCCCCAGCCUGGUAUGGAGU | | |
| CCAGUCUAAGCAGAGAGACUGAUGGGCAGGGGAGGUGGGACCUUCAGCCCAGGGUCCACUGUG | | |
| GGGGCAGAGGGAGUGGCAGAGACACCGGGGUUCCUUCCCCUAAUGGGUCACCUUCUCUUGACC | | |
| UUUCAGAAUAUGUGAACCAGCCAGAUGUUCGGCCCCAGCCCCCUUCGCCCCGAGAGGGCCCUC | | |
| UGCCUGCUGCCCGACCUGCUGGUGCCACUCUGGAAAGGCCCAAGACUCUCUCCCCAGGGAAGA | | |
| AUGGGGUCGUCAAAGACGUUUUUGCCUUUGGGGGUGCCGUGGAGAACCCCGAGUACUUGACAC | | |
| CCCAGGGAGGAGCUGCCCCUCAGCCCCACCCUCCUCCUGCCUUCAGCCCAGCCUUCGACAACC | | |
| UCUAUUACUGGGACCAGGACCCACCAGAGCGGGGGGCUCCACCCAGCACCUUCAAAGGGACAC | | |
| CUACGGCAGAGAACCCAGAGUACCUGGGUCUGGACGUGCCAGUGUGAACCAGAAGGCCAAGUC | | |
| CGCAGAAGCCCUGAUGUGUCCUCAGGGAGCAGGGAAGGCCUGACUUCUGCUGGCAUCAAGAGG | | |
| UGGGAGGGCCCUCCGACCACUUCCAGGGGAACCUGCCAUGCCAGGAACCUGUCCUAAGGAACC | | |
| UUCCUUCCUGCUUGAGUUCCCAGAUGGCUGGAAGGGGUCCAGCCUCGUUGGAAGAGGAACAGC | | |
| ACUGGGGAGUCUUUGUGGAUUCUGAGGCCCUGCCCAAUGAGACUCUAGGGUCCAGUGGAUGCC | | |
| ACAGCCCAGCUUGGCCCUUUCCUUCCAGAUCCUGGGUACUGAAAGCCUUAGGGAAGCUGGCCU | | |
| GAGAGGGGAAGCGGCCCUAAGGGAGUGUCUAAGAACAAAAGCGACCCAUUCAGAGACUGUCCC | | |
| UGAAACCUAGUACUGCCCCCCAUGAGGAAGGAACAGCAAUGGUGUCAGUAUCCAGGCUUUGUA | | |
| CAGAGUGCUUUUCUGUUUAGUUUUUACUUUUUUUGUUUUGUUUUUUUAAAGAUGAAAUAAAGA | | |
| CCCAGGGGGAGAAUGGGUGUUGUAUGGGGAGGCAAGUGUGGGGGGUCCUUCUCCACACCCACU | | |
| UUGUCCAUUUGCAAAUAUAUUUUGGAAAACAGCUAGGCACCGGCCUAUGUCUGGGGGUGGCUC | | |
| UGUGCC | | |
| CAGA | ERBB2 response | |
| | element | |

TABLE 1G
Tabular depiction of genetic themes for ribosome fate switch and mRNA destabilization

| | desARE3′UTR | desARE3′UTR | 3′UTRMYC1- | |
|---|---|---|---|---|
| | ERBB2-3 | ERBB2-30 | 18 | |
| LSM10 | TRUE | TRUE | TRUE | premRNA DCP1A binding |
| RPL11 | TRUE | TRUE | TRUE | ribosome turnover and biogenesis |
| MRTo4 | TRUE | | TRUE | ribosome turnover and biogenesis |
| NME1 | TRUE | TRUE | TRUE | RNA binding and Damage |
| EXOSC4 | TRUE | TRUE | TRUE | 3′UTR stalling & degradation, |
| | | | | RNA exosome exoribonuclease |
| | | | | unstable 3′UTR |
| EXOSC5 | TRUE | TRUE | TRUE | RNA exosome exoribonuclease |
| | | | | unstable 3′UTR |
| RPL3 | TRUE | TRUE | TRUE | Nascent polypeptide |
| RPL24 | TRUE | TRUE | | ribosome turnover and biogenesis |
| PELO | | TRUE | TRUE | 3′UTR stalling & degradation |
| EMG1 | TRUE | TRUE | | Nacent polypeptide |
| RPL4 | TRUE | TRUE | | Nascent polypeptide |
| NEMF | TRUE | TRUE | | Nascent polypeptide stalled |
| RPL5 | TRUE | TRUE | | Nascent polypeptide |
| HBS1L | | TRUE | | 3′UTR stalling & degradation |
| DDX56 | TRUE | | TRUE | RNA alteration |
| NOL6 | | | TRUE | Nascent premRNA and RNA exosome |
| ZNF598 | | | TRUE | in frame stalling of ribosomes, |
| (Hel2) | | | | and senses slow ribosomes and |
| | | | | diribosomes that collides due |
| | | | | to slow kinetics |
| SMG9 | | | TRUE | Nonsense mediated decay, premature |
| | | | | termination codon, in frame |
| | | | | stalled ribosome |
| ABCE1 | | | | 3′UTR stalling & degradation |
| ERF1/ETF1 | | | | Premature termination, stalled |
| | | | | ribosomes in frame, NMD UPF1 |
| INTS6 | | | | 3′ RNA processing, |
| (DDX26) | | | | RNA helicase |
| SKIV2L | | | | RNA degradosome 3′UTR stalling & |
| (SKIC2) | | | | degradation and PELOTA, HBS1L, |
| | | | | ABCE1, RNA altertion |

### Example 1B: Results

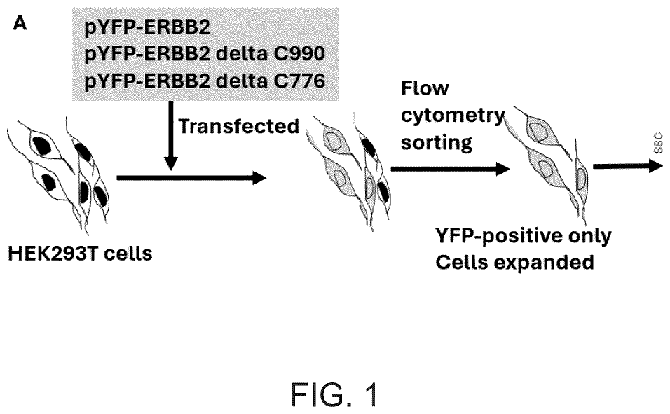

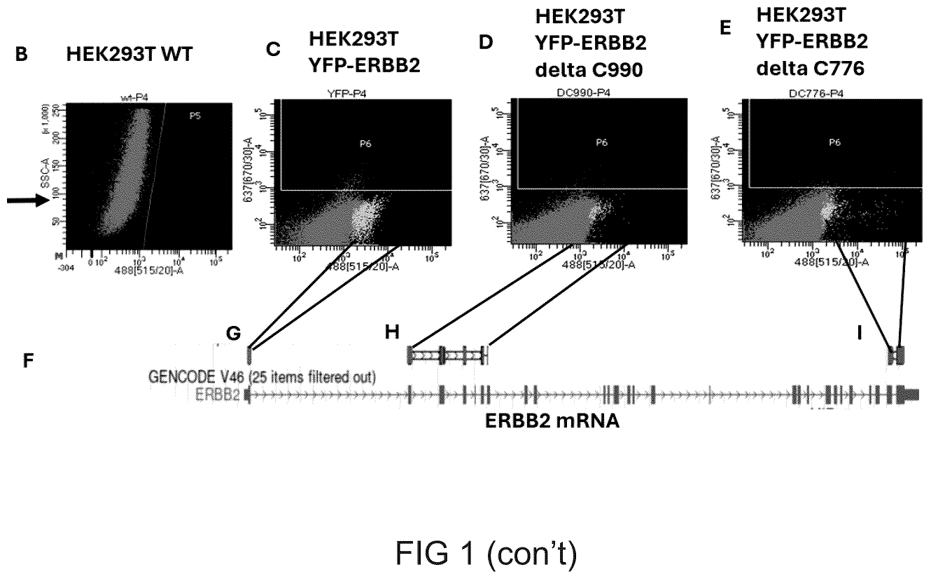

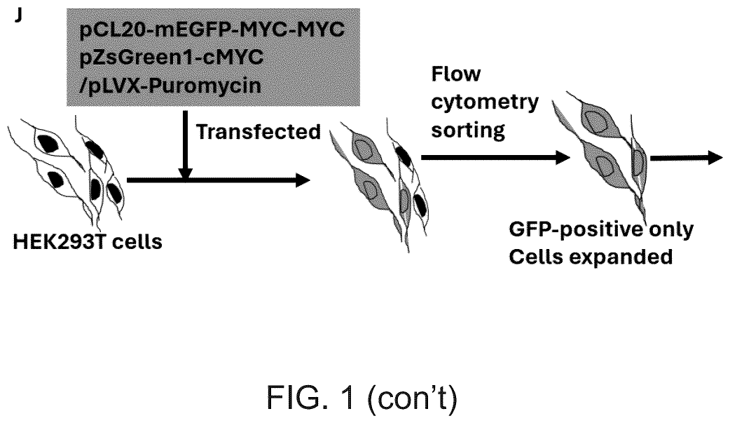

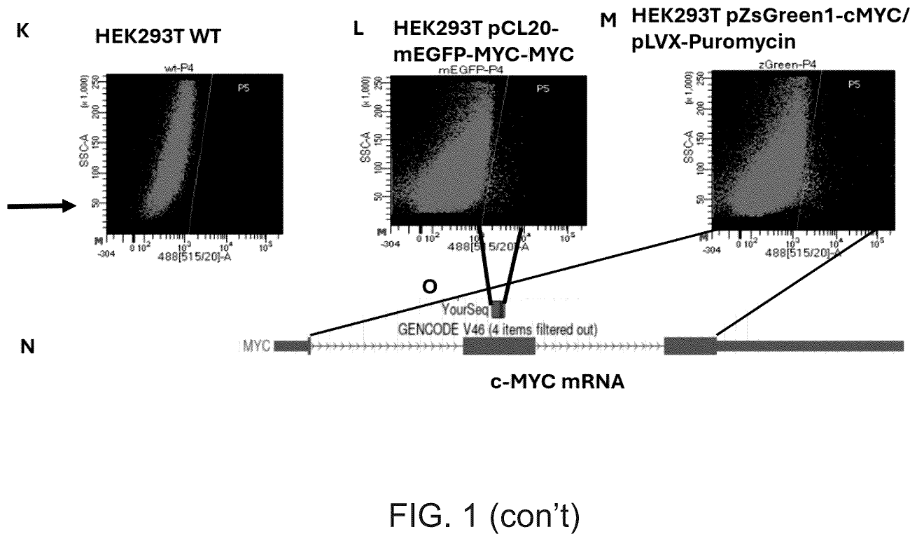

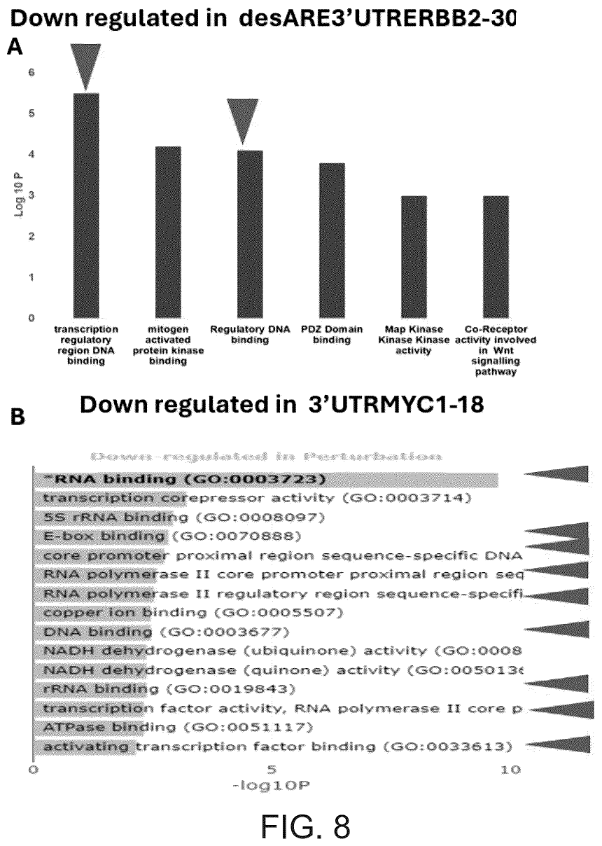

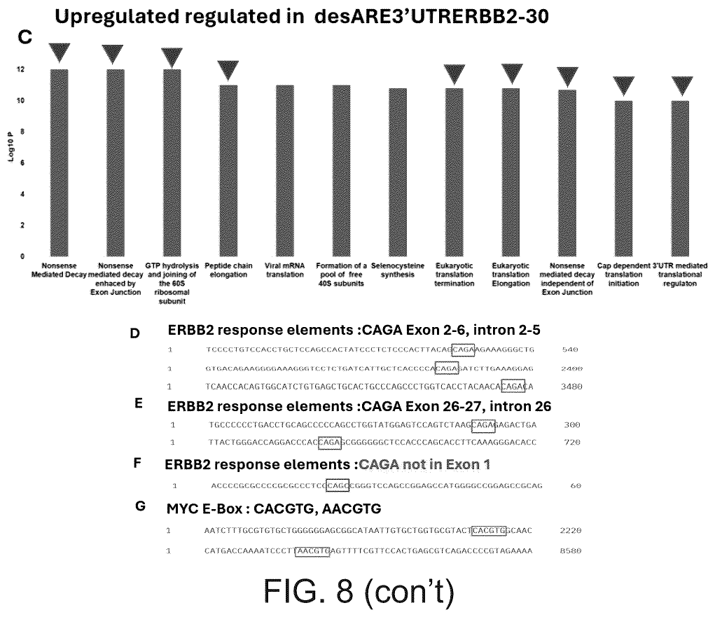

The destabilized 3′UTR mRNA recognition of the target mRNA site is sequence specific. We developed the 3′UTR mRNA destabilizing drugs of the ERBB2 and the c-MYC, which degraded the target mRNA specifically, the ERBB2 and the c-MYC, respectively. The gene ontology shows that it recognizes sequence specific regulatory elements (FIG. 8A-B) for the ERBB2 and the c-MYC. We next sought to identify this sequence specific element of the mRNA recognition site for the destabilized 3′UTR mRNA function. We used various plasmid constructs expressing under GFP or YFP the various portions of the target mRNA cDNA sequences. Specifically, for the ERBB2 targeting, we obtained the pYFP-ERBB2 sequence (26) (which expresses the first ERBB2 exon), the pYFP-ERBB2 delta C990 (which expresses the ERBB2 exons 2-7 and intron 2-6) and the pYFP-ERBB2 delta C776 (which expresses ERBB2 exons 26-27 and intron 26) (FIG. 1A-I). For the c-MYC targeting, we obtained the pCL20-mEGFP-MYC-MYC (which expresses 132 bp region within the c-MYC exon 2-Chr 8:127738568-127738691) and the pZsGreen1-cMYC/pLVX-puromycin (which expresses the c-MYC exon 1-3 and the introns 1-2) (FIG. 1J-O). We transfected these plasmids individually into the HEK293T cells and we sorted the transformed cells by flow cytometry to collect only YFP or GFP (green) positive cells respectively (FIG. 1A, 1J). The untransduced HEK293T cells (FIG. 1B, 1K) were used as controls. The YFP positive cells (FIG. 1C, D, E) and the GFP only positive cells (FIG. 1L, 1M) were thereby separated. The sorted cells were then expanded and used for experiments as described in the methods.

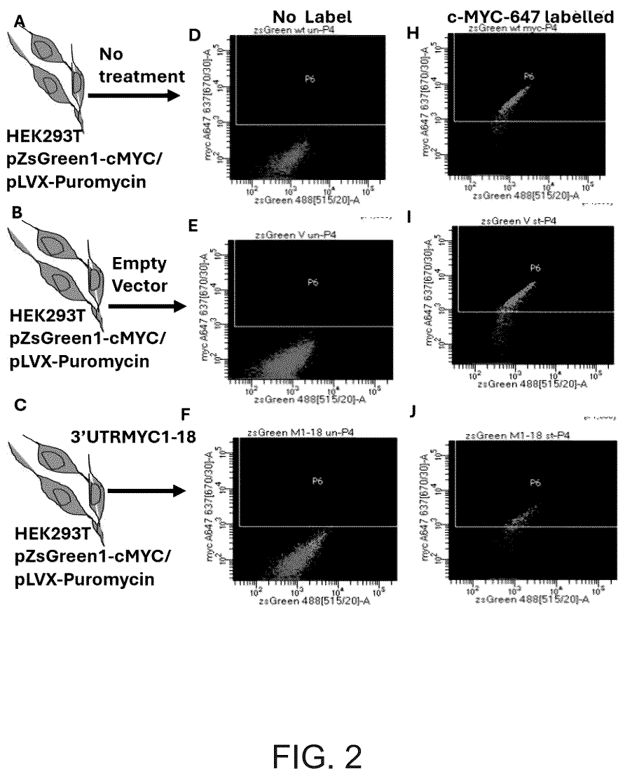

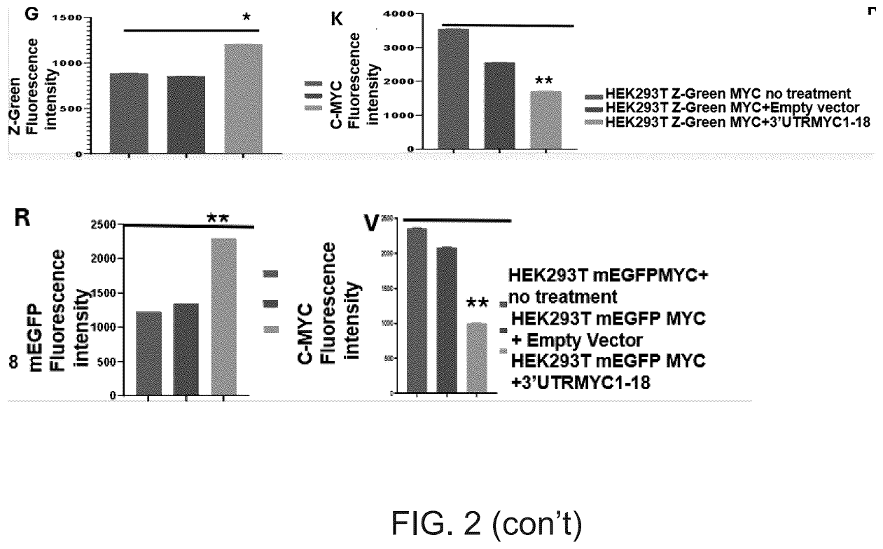

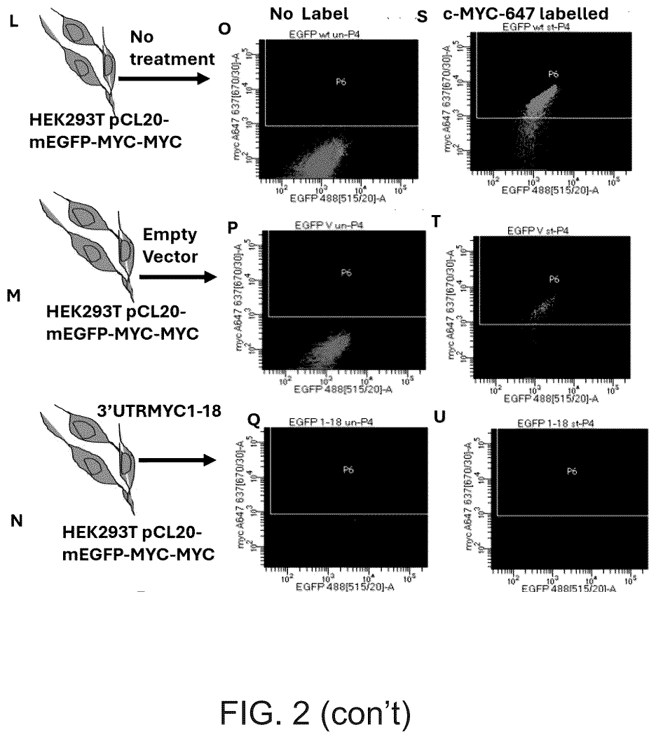

One aim was to understand if the MYC mRNA destabilizing drug (3′UTRMYC1-18) recognized the endogenous c-MYC mRNA element specifically. We treated the expanded pZsGreen1-cMYC/pLVX-puromycin into 3 groups as follows: 1) no treatment (FIG. 2A), 2) empty vector treated (FIG. 2B), 3) 3′UTRMYC1-18 treated (FIG. 2C). Both treated and untreated cells were harvested and split into unlabeled and c-MYC 647 labelled cells. The cells were then analyzed on the flow cytometry machine. We found that the untreated groups (FIG. 2D, E, F) expressed the GFP as expected. These results were quantified as shown in FIG. 2G. Next, we analyzed the c-MYC 647 treated cells. We found that there were no differences in the MYC expression between the untreated and empty vector cells (FIG. 2H, I, K). Notably, 3′UTRMYC1-18 down regulated the c-MYC expression 3-fold compared to the controls (FIG. 2J, K). These results mean that the mRNA destabilizing drug, 3′UTRMYC1-18, specifically recognized the c-MYC exon 1-3 and the introns 1-2 mRNA elements and specifically down regulated their expression.

We extended these findings by treating the expanded pCL20-mEGFP-MYC-MYC into 3 groups as follows: 1) no treatment (FIG. 2L), 2) empty vector treated (FIG. 2M), and 3)3′UTRMYC1-18 treated (FIG. 2N). Both treated and untreated cells were harvested and split into unlabeled and c-MYC 647 labelled cells. The cells were then analyzed on the flow cytometry machine. We found that the untreated groups (FIG. 2O, P, Q) expressed the GFP as expected. These results were quantified as shown in FIG. 2R. We analyzed the c-MYC 647 treated cells, and we found that there were remarkably elevated levels of MYC expression in the untreated and empty vector cells (FIG. 2S, T, R). The c-MYC mRNA destabilizing drug treated cell down regulated the c-MYC expression 2.5-fold compared to the controls (FIG. 2U, V). The findings show that the c-MYC mRNA destabilizing drug, 3′UTRMYC1-18, specifically recognized the 132 bp region within the c-MYC exon 2-Chr 8:127738568-127738691 and down regulated it.

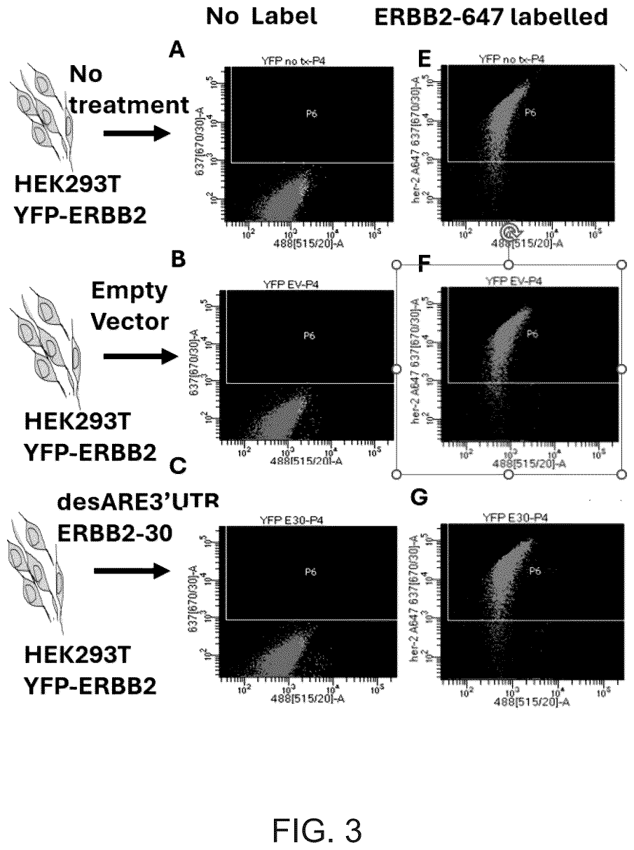

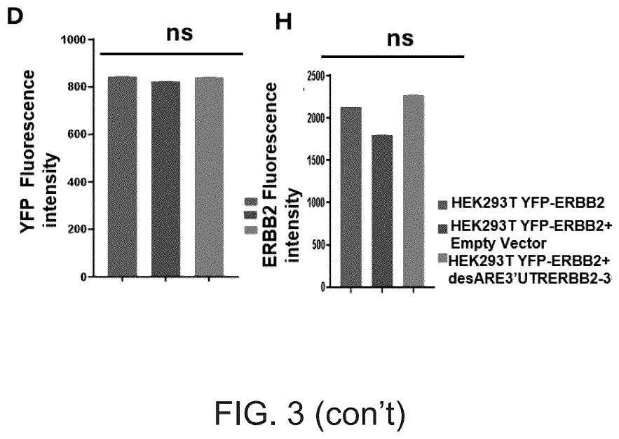

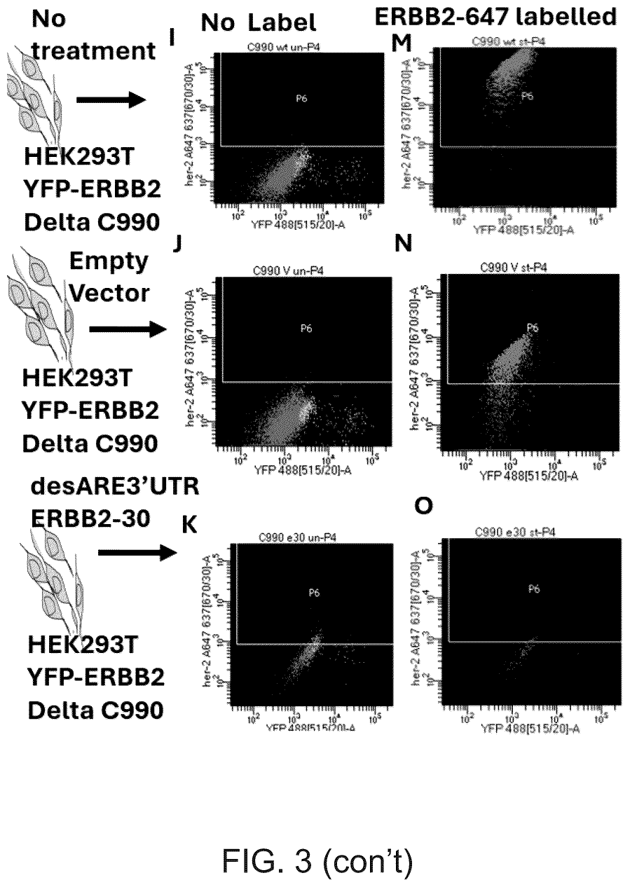

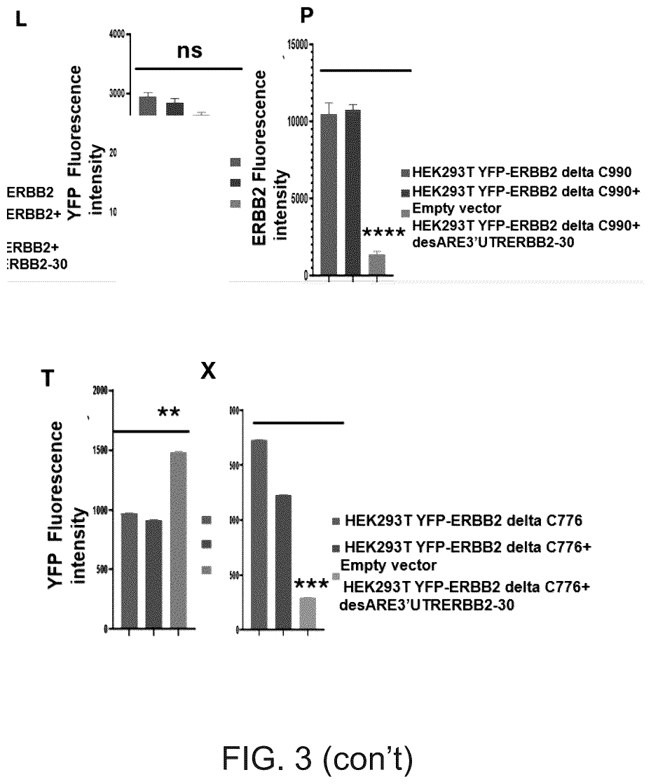

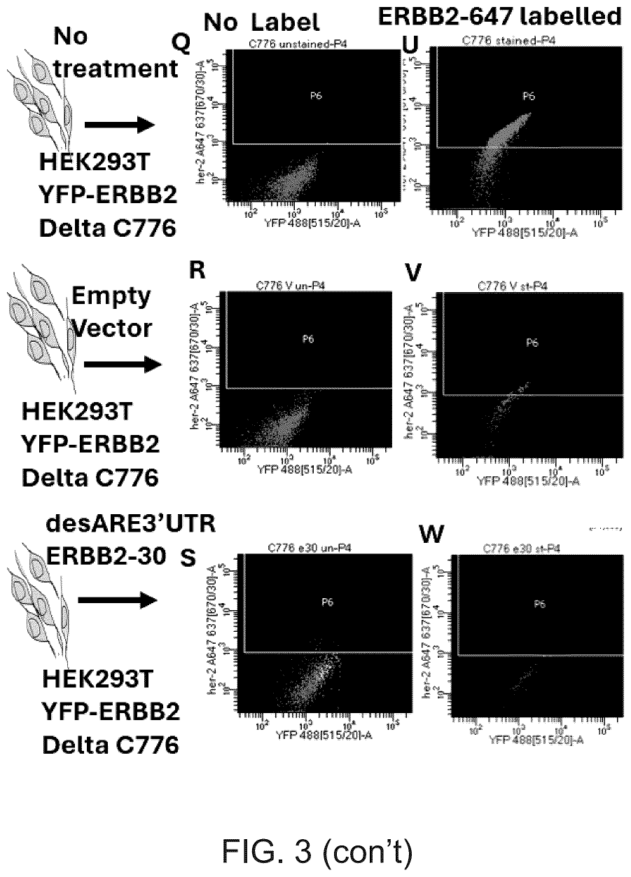

Our next aim was to validate the versatility of the mRNA destabilizing drug specific recognition of the target endogenous mRNA sequences. We evaluated the ERBB2 system with the ERBB2 mRNA destabilizing drugs. We created the 3′UTR ERBB2 mRNA destabilizing drug called the desARE3′UTRERBB2-30 (14-16) which destabilized the ERBB2 mRNA and triggered its specific transcript degradation across multiple ERBB2 driven cancers. To find out how this drug specifically recognizes the endogenous ERBB2 mRNA, we expanded the HEK293T cells expressing the pYFP-ERBB2 and split it into 3 groups: 1) no treatment (FIG. 3A), 2) empty vector treated (FIG. 3B), and 3) desARE3′UTRERBB2-30 treated (FIG. 3C). We found that the pYFP-ERBB2 transduced cells express YFP by the flow cytometry. These results were quantified as shown in FIG. 3D. We then labelled the same cells (FIG. 3A-C) with ERBB2-647 an antibody against ERBB2. We found that the ERBB2 expression did not change in the treated cells compared to the controls analyzed by the flow cytometry (FIG. 3E, 3F, G). These results were quantified as shown in FIG. 3H. These findings indicate that the ERBB2 mRNA destabilizing drug does not recognize the first ERBB2 exon.

Next, we expanded the HEK293T cells transduced with the pYFP-ERBB2 delta C990 and we split the cells into 3 groups: 1) no treatment (FIG. 3I), 2) empty vector treated (FIG. 3J), and 3) desARE3′UTRERBB2-30 treated (FIG. 3K). We found that the transduced cells expressed YFP by the flow cytometry. The results were quantified as shown in FIG. 3L. We next sought to assess ERBB2 expression in the same cells (FIG. 3I, J, K). To do this, we labelled the same cells with an ERBB2-647 antibody. We found that the desAR3′UTRERBB2-30 completely abolished the ERBB2 expression compared to the controls in the flow cytometry (FIG. 3M, N, O). The ERBB2 level is decreased 174-fold by desARE3′UTRERBB2-30 in the treated cells as compared to the controls. These results demonstrate that the ERBB2 mRNA destabilizing drug specifically recognizes the endogenous ERBB2 mRNA elements on the ERBB2 exons 2-7 and intron 2-6 and this drug achieves downregulation based on this recognition.

Lastly, we expanded HEK293T cells transduced pYFP-ERBB2 delta C776 and split it into 3 groups: 1) no treatment (FIG. 3Q), 2) empty vector treated (FIG. 3R), and 3) desARE3′UTRERBB2-30 treated (FIG. 3S). We found that the transduced cells expressed YFP by the flow cytometry. The results were quantified as shown in FIG. 3T. We then stained the same cells (FIG. 3Q, 3R, 3S) with an ERBB2-647 antibody to assess ERBB2 expression. We found that the ERBB2 mRNA destabilizing drug, desARE3′UTRERBB2-30 downregulated ERBB2 expression 5.8-fold compared to the controls (FIG. 3U, V, W) as analyzed by flow cytometry. The results were quantified as shown in FIG. 3K. Our findings indicate that the ERBB2 mRNA destabilizing drug directly and specifically recognizes the ERBB2 exons 26-27 and intron 26 and this drug achieves downregulation based on this recognition.

Accordingly, we have shown that the mRNA destabilizing drugs are specific and bind to the target mRNA sequences. We next assessed gene ontology terms to show that the mRNA destabilizing drugs recognized specific DNA and regulatory elements (FIGS. 8A and B, marked with arrows). To identify the relevant sequence elements, we searched for the ERBB2 response elements having a CAGA motif. We found three of these elements in the ERBB2 exons 2-7 and introns 2-6 (FIG. 8D, marked with boxes). Two of the CAGA elements were identified on ERBB2 exons 26-27 and intron 26 (FIG. 8E, marked with boxes) but not in the ERBB2 exon 1 (FIG. 8F, marked with a box). This explains why the ERBB2 exons 2-7 and introns 2-6 and the ERBB2 exon 26-27, introns 26 are recognized by the ERBB2 mRNA, whereas the ERBB2 exon 1 is not. We extended our analysis to c-MYC and found that the MYC mRNA destabilizing drug recognized the MYC E-box elements CACGUG and AACGUG on MYC (FIG. 8G, marked with boxes). Taken together, our results demonstrate that the mRNA destabilizing drugs target mRNAs in a sequence specific manner. This provides a direct indication of how the endogenous transcripts are targeted and downregulated by the mRNA destabilizing drugs.

Identification of the molecular ribosome fate switch mediators in mRNA transcript degradation by the specific 3′UTR destabilization. We have shown that the destabilized mRNA is target mRNA sequence specific. We then asked how specific sequence recognition leads to the specific transcript degradation. Our aim was to identify the molecular switch that mediates the ribosome fate switch and the transcript degradation by the engineered 3′UTR mRNA destabilizing drugs. To assess this, we used an unbiased approach based on genome scale analysis of elevated mRNA of the genes that were upregulated in the destabilized cells regardless of the destabilized target gene compared to controls of WT (wild type) and vector controls. We used three destabilizing model systems: 1) 3′UTRMYC1-18 for c-MYC, 2) desARE3′UTRERBB2-3 and the desARE3′UTRERBB2-30 for ERBB2, 3) 3′UTRTEAD1-T5 for TEAD1. Three model cell lines were also used: NCI H1975 (lung cancer), MDAMB231 (triple negative breast cancer-Caucasian descent woman) and MDAMB468 (triple negative breast cancer-African descent woman) (FIG. 9A). We extracted total RNA and prepared the library and sequenced on the Illumina Next generation sequencer. The RNA seq analysis revealed key genetic pathways that were upregulated. This included: mRNA 3′UTR end stalling, exon mRNA stalling, ribosome in frame stalling and ribosome synthesis and turnover, and nascent polypeptide stalling (FIG. 8C, 9B). Most of the protein identified fall within the premRNA DCP1A binding, ribosome turnover and biogenesis, RNA binding and damage, 3′UTR stalling and degradation, RNA exosome, RNA exoribonuclease, unstable UTR, and nascent polypeptide (FIG. 9B).

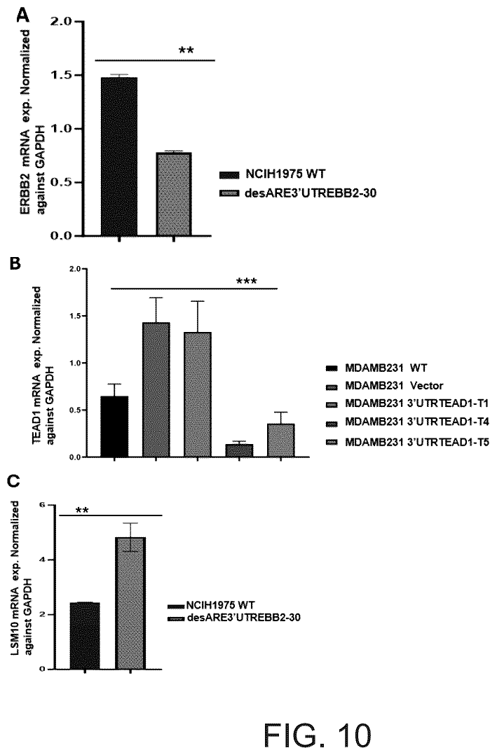

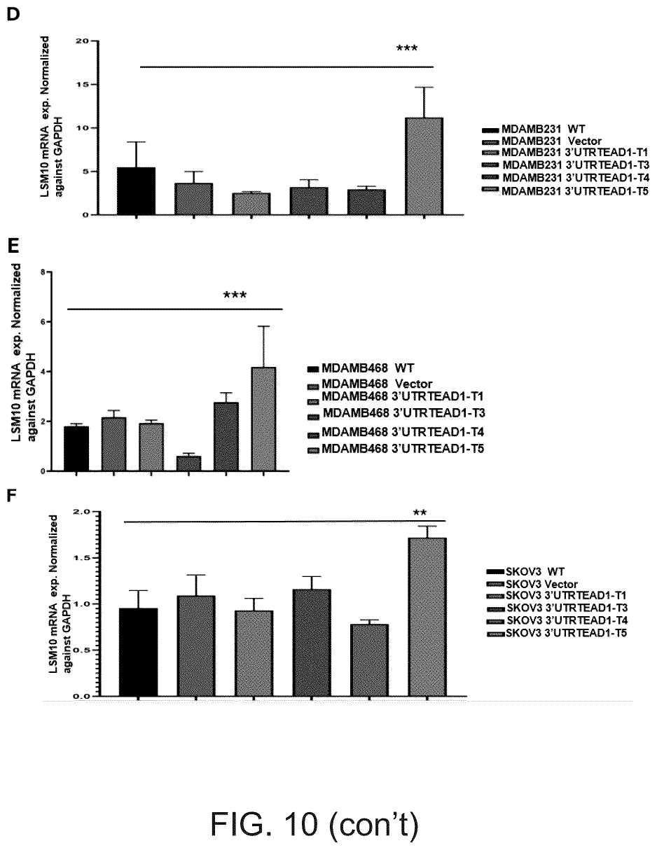

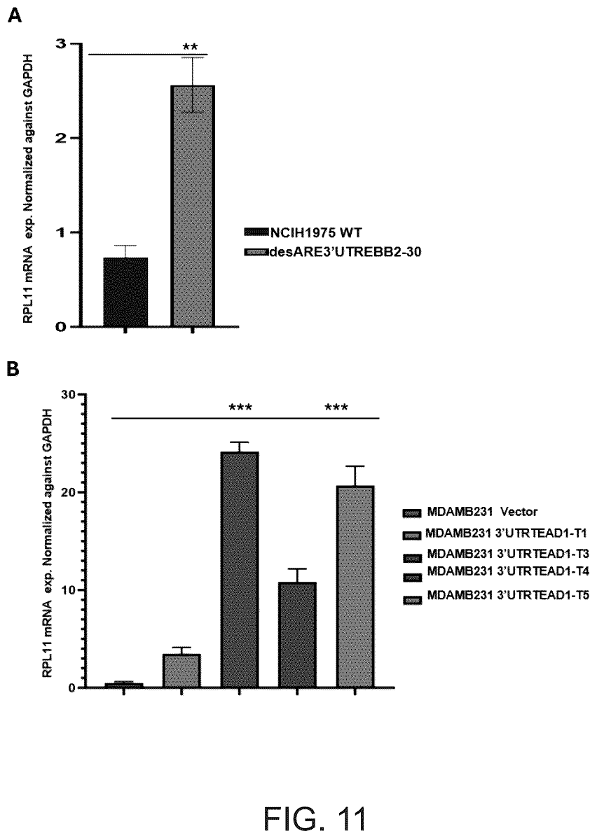

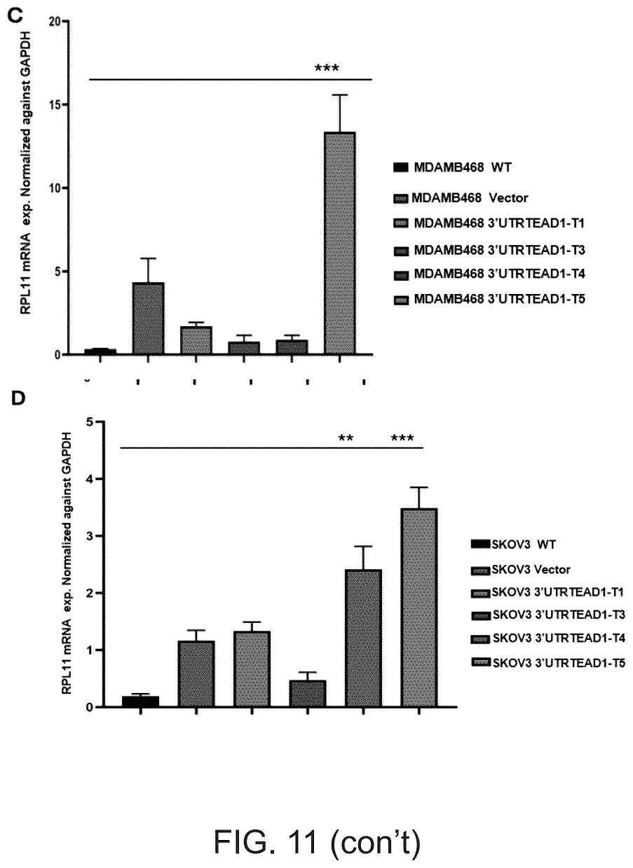

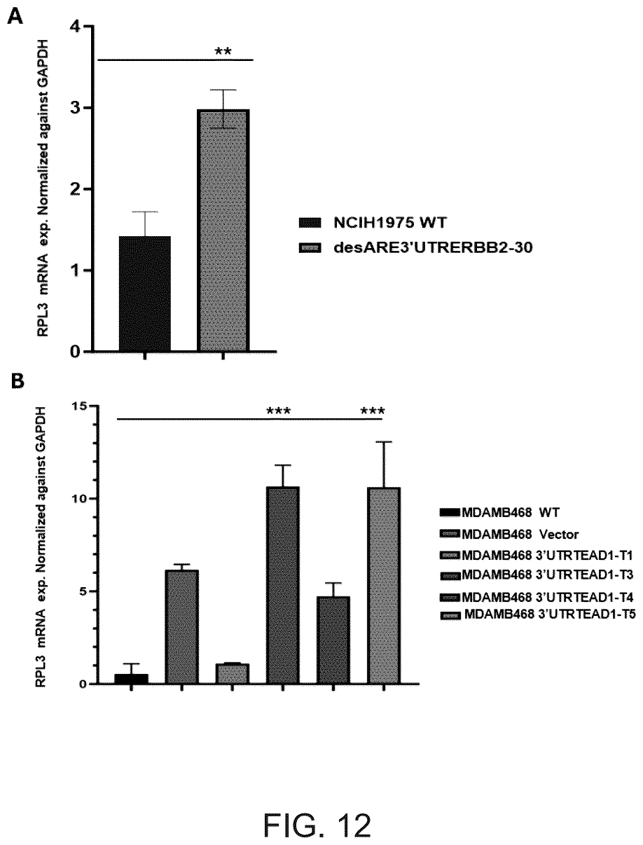

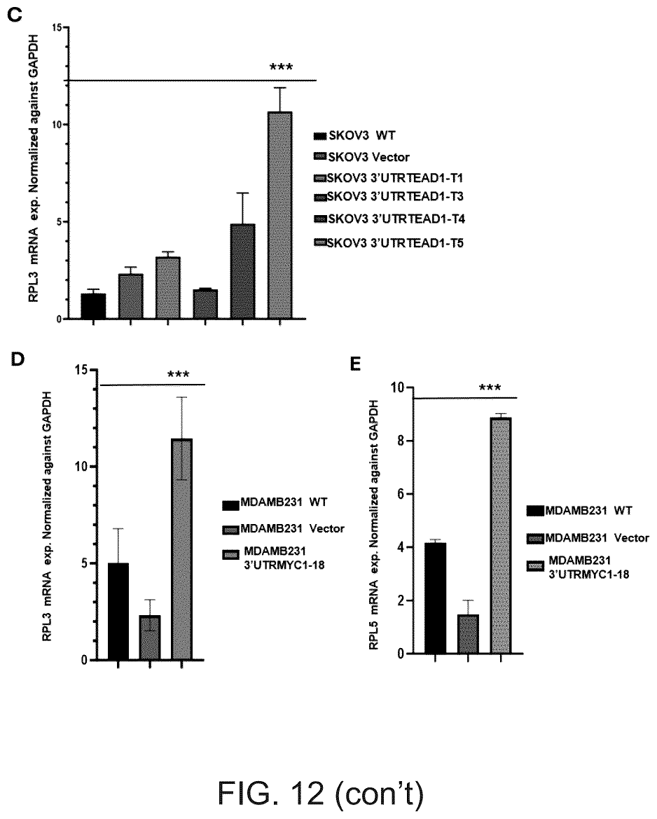

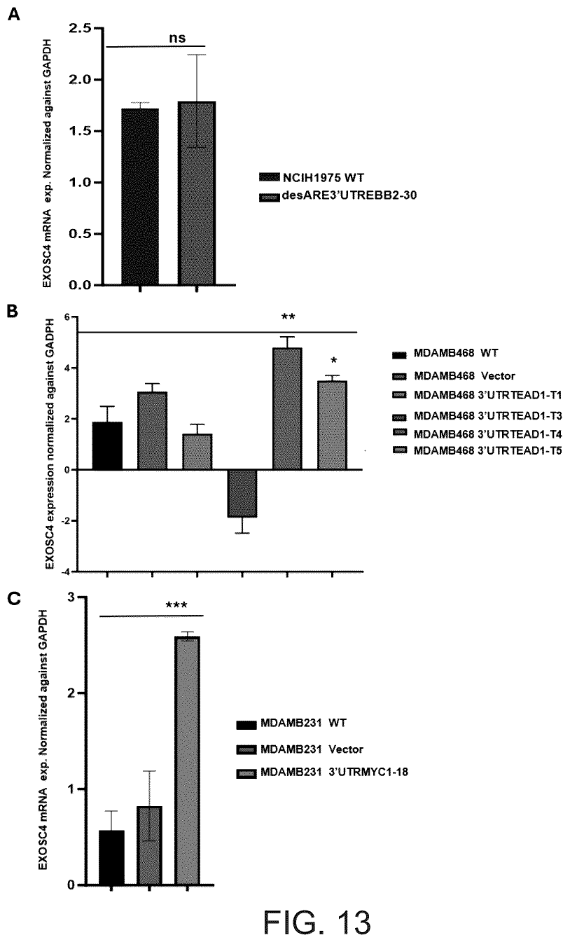

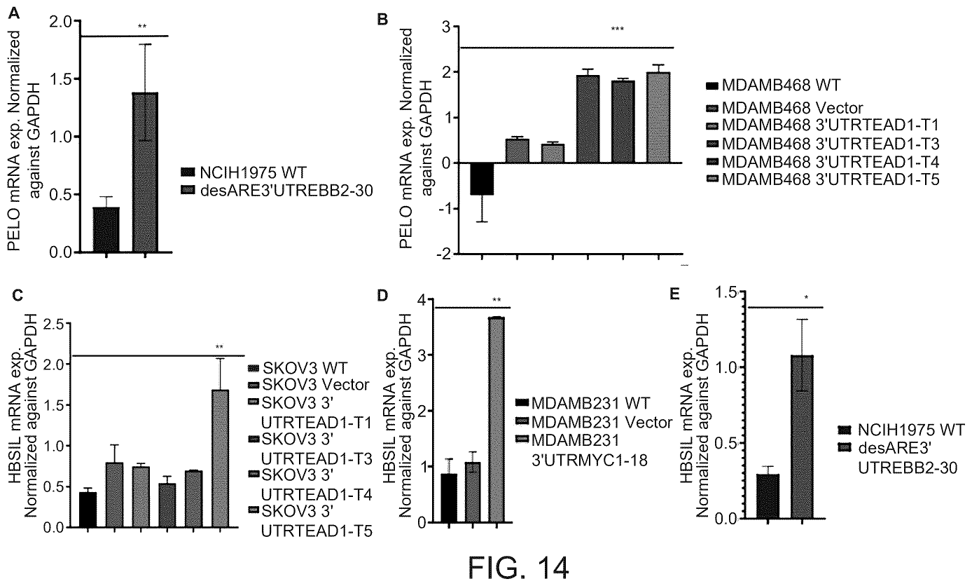

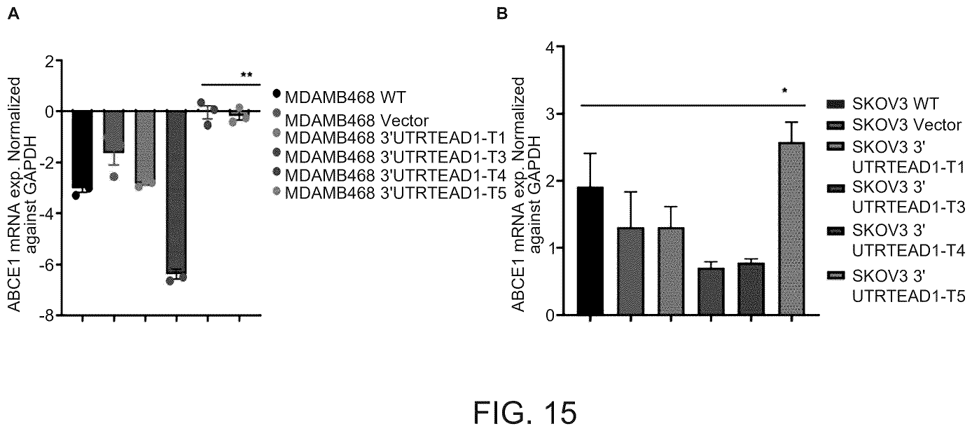

To validate the genes identified as elevated in the mRNA seq data, we performed an unbiased quantitative reverse transcript PCR on the genes identified from the three destabilized model system of the desARE3′UTRERBB2-30 (FIG. 10A-destabilized ERBB2), 3′UTRTEAD1-T5 (FIG. 10B-destabilized TEAD1) and 3′UTRMYC1-18. We validated the elevated expression LSM10 in the destabilized cells across multiple cancers NSCLC, MDAMB231 (triple negative breast cancer-Caucasian descent woman), MDAMB468 (triple negative breast cancer-African descent woman), and SKOV3 (ovarian cancer) (FIG. 10C, D, E, F). Next, we assayed for the elevated expression levels of the RPL11 in the destabilized model system of the desARE3′UTRERBB2-30, 3′UTRTEAD1-T5 and we found in the RPL1 Ito be elevated in the destabilized cells of NSCLC, MDAMB231, MDAMB468 and SKOV3 (FIG. 11A-D). We extended our investigation into RPL3 and could show that they were elevated in expression in destabilized system TEAD1, MYC and ERBB2 (FIG. 12A-D) and RPL5 in the destabilized MYC system (FIG. 12E). We also found the upregulated transcript of the EXOSC4 (FIG. 13A-C), the PELO (FIG. 14A-B) and the HBSIL and the ABCE1 (FIG. 14C-E, FIG. 15A-B) in the destabilized model system of 3′UTRMYC1-18, TEAD1 and ERBB2 in cancers. The expression levels of SMG9, MRT04, ERF1/ETF1, EMG1, RPL4, ZNF598 (Hell), NME1, INTS6 (DDX26), RPL24, SKIV2L (SKIC2), DDX56 and NOL6 were low on validation qPCR across different model destabilized systems and thus they were screened off and were not selected as further candidates for investigation.

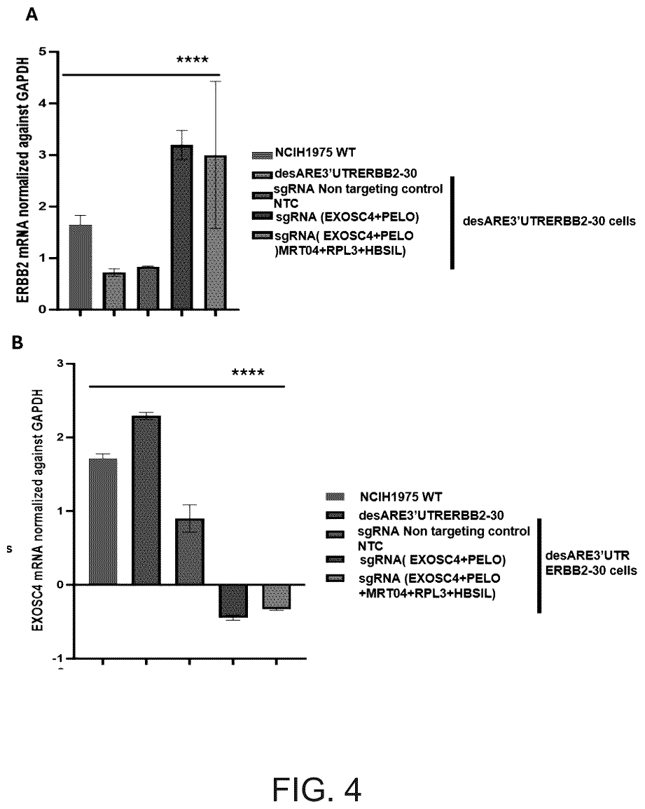

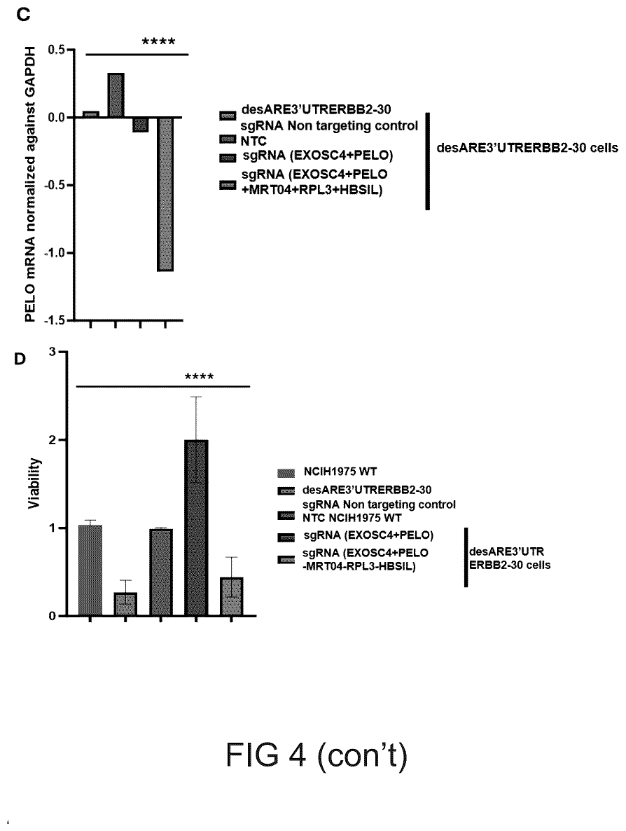

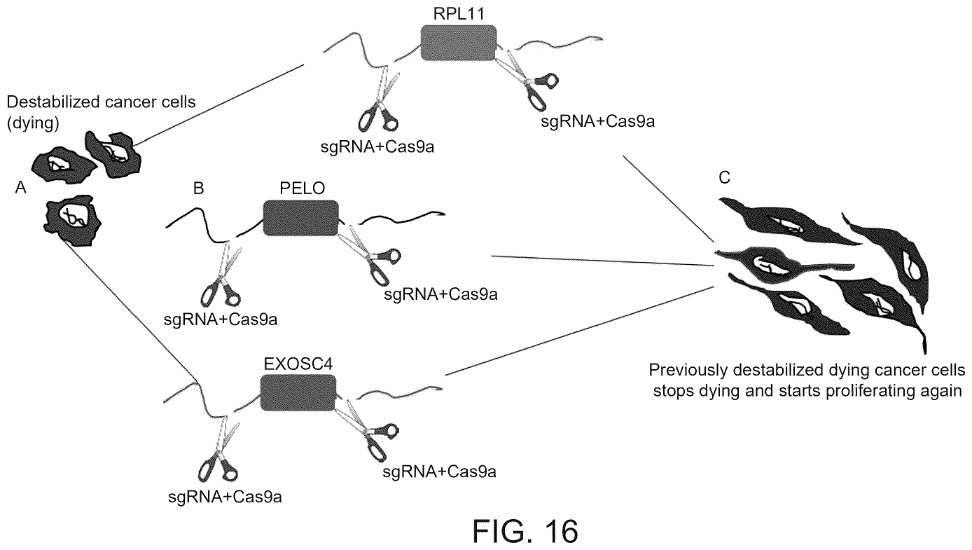

CRISPR Cas9a knockout of the PELO, EXOSC4 and RPL11, RPL3 validates their function as ribosome fate switch molecular regulators mediating the destabilizing engineered 3′UTR specific degradation of transcripts (oncogenic). We have identified the PELO, EXOSC4, RPL11 and RPL3 transcripts as being upregulated uniformly in the presence of the mRNA destabilizing constructs across many cancers. To confirm that these genes are implicated in the biology and regulation of the ribosome fate switch, we performed a CRISPR-Cas9a knockout of these genes alone, in twos or in fours (FIG. 4A). After four days, we assessed for the loss of the transcript. We found loss of the PELO and EXOSC4 in the cells destabilized with desARE3′UTRERBB2-30 constructs compared the controls (WT, vector and the sgRNA non targeting) (FIG. 4B, C). Subsequently, we assessed the ERBB2 mRNA expression and cell viability in the destabilized cells with the EXOSC4 and PELO knockouts. We found the marked restoration of the ERBB2 mRNA expression in the cells where EXOSC4 and PELO were knocked out, as compared to the controls (FIG. 4A). We found also increased viability of the cancer cells more than WT controls (FIG. 4D). These results implicated EXOSC4 and PELO as being directly involved in the mechanism of the ribosome fate switch in the destabilized cancer cells. Our findings are illustrated in FIGS. 16A-C.

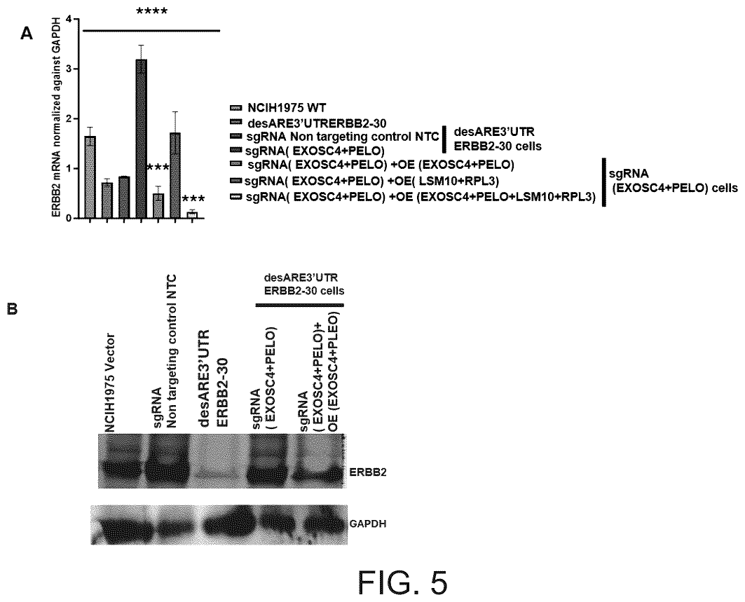

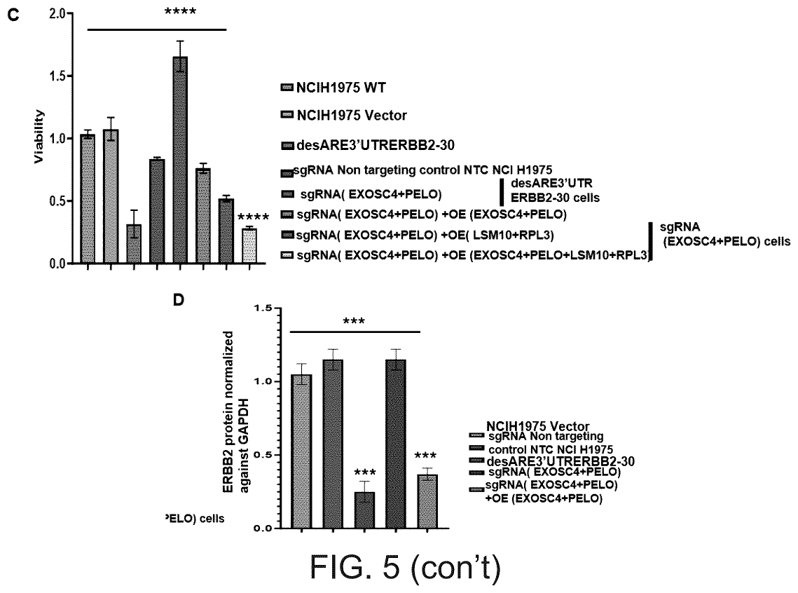

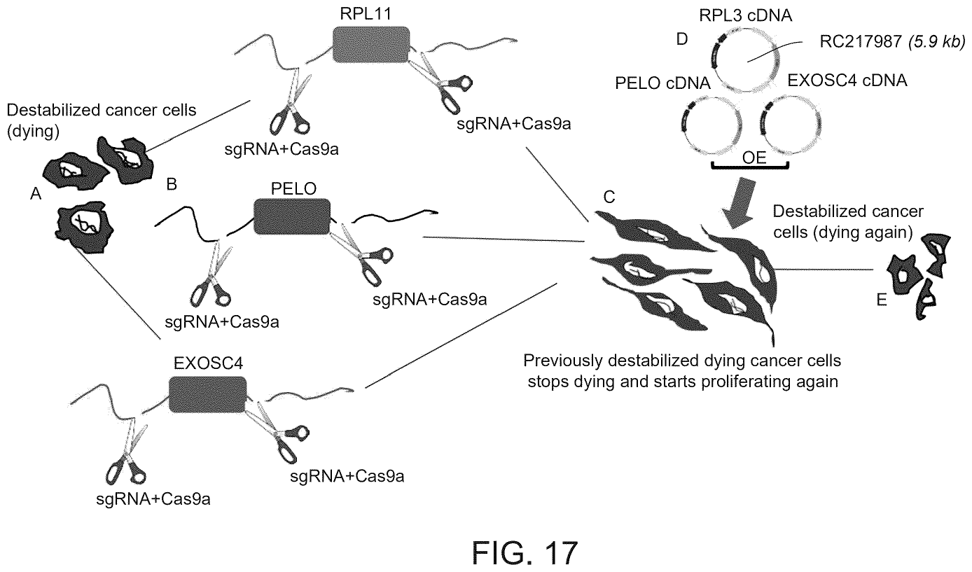

The gain of function over expression of the EXOSC4, PELO in CRISPR KO (EXOSC4 and PELO) restores the transcript degrading function of the engineered destabilized 3′UTR. To confirm that EXOSC4 and PELO were the molecules controlling the switch from the ribosomal translation to degradation, we performed gain of function experiments. We overexpressed 1) EXOSC4 and PELO, 2) LSM10 and RPL3, or 3) EXOSC4+PELO+LSM10+RPL3 in the cells in which EXOSC4 and PELO were knocked out. We found the down regulation of the ERBB2 mRNA and protein in the KO cells with overexpressed the PELO and EXOSC4 comparable to the desARE3′UTRERBB2-30 treated cells (FIG. 5A-B). The over expression of the LSM10 and RPL3 had no impact on the ERBB2 mRNA expression. However, the over expression of the EXOSC4, PELO, LSM10 and RPL3 downregulated the ERBB2 mRNA more than the desARE3′UTRERBB2-30 cells and the EXOSC4 and PELO over expressing cells (FIG. 5A, C). These data clearly demonstrate that the EXOSC4 and PELO are the key regulators of the ribosome fate switch in the presence of the mRNA destabilizing construct, and that LSM10 and RPL3 play only supporting roles. The findings are illustrated in the FIG. 17A-E.

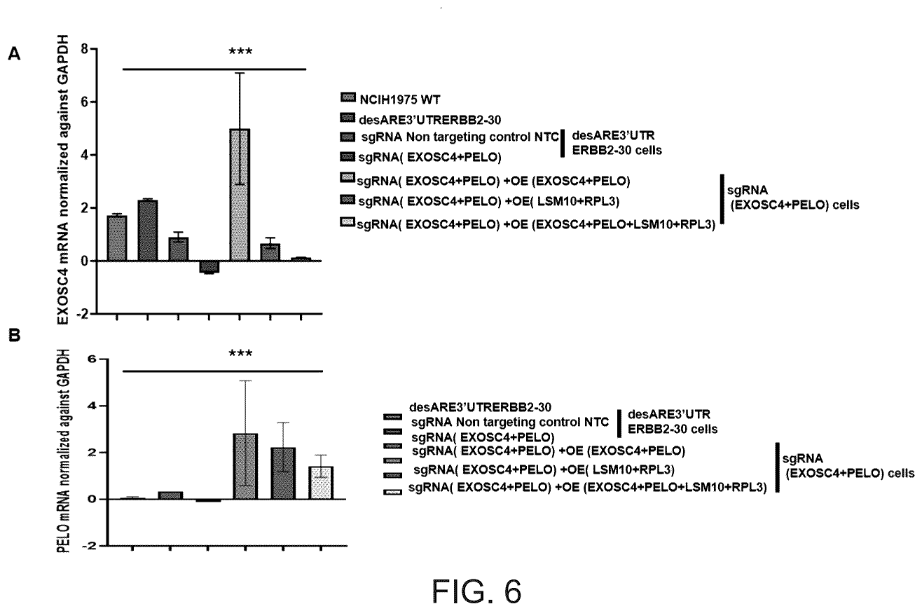

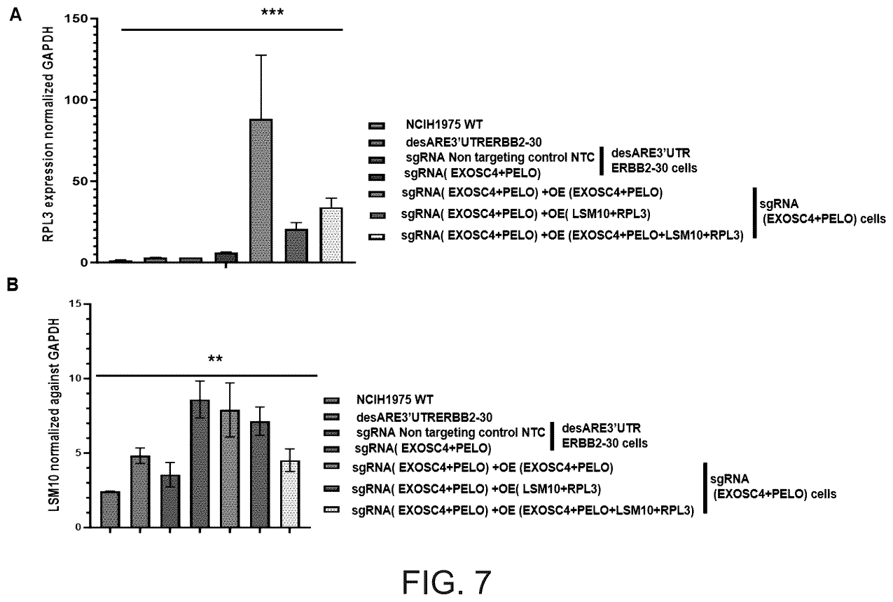

EXOSC4 and PELO co-operate in a 1:1 stoichiometric ratio in the regulation of the ribosome fate switch by the destabilized 3′UTR constructs. Next, we investigated the stoichiometry with which the PELO and EXOSC4 co-operate to mediate ribosomal switch from translation to degradation. We assayed the expression levels of the EXOSC4 and PELO in the KO cells overexpressing EXOSC4 and PELO. We found that the relative EXOSC4 and PELO mRNA stoichiometry ratio is 1:1 (FIG. 6A-B). RPL3 expression is elevated greater than 100-fold and LSM10 expression is elevated 1.5-fold (FIG. 7A-B). Taken together, this data shows the relative ratios of EXOSC4 and PELO, as well as RPL3 and LSM10, involved in switching ribosomal fate to degradation in the presence of the mRNA destabilizing drugs.

Morphological phenotypic cellular changes of changing translating mRNA to degrading and vice versa. Our next aim was to correlate the molecular phenomena to their cellular phenotypes. To do this, we documented the images of the cells under different treatments and knockout and over expression conditions. We assessed: 1) proliferating untreated NSCLC as shown in FIG. 18A; 2) proliferating vector and sgRNA non targeting control NSCLC as shown in FIG. 18B-C; 3) destabilized and dying desARE3′UTRERBB2-30 treated cells as shown in FIG. 18D. The sgRNA PELO and EXOSC4 knockout cells treated with desARE3′UTRERBB2-30 exhibit proliferation as shown in FIG. 18E. These levels are higher than the WT NSCLC as shown in FIG. 18A. The sgRNA PELO and EXOSC4 knockout cells treated with desARE3′UTRERBB2-30 but having overexpression of PELO and EXOSC4 exhibit a significant reduction in proliferation (FIG. 18F). This reduction is comparable to the destabilized and dying cells seen in the desARE3′UTRERBB2-30 treated cells as shown in FIG. 18D.

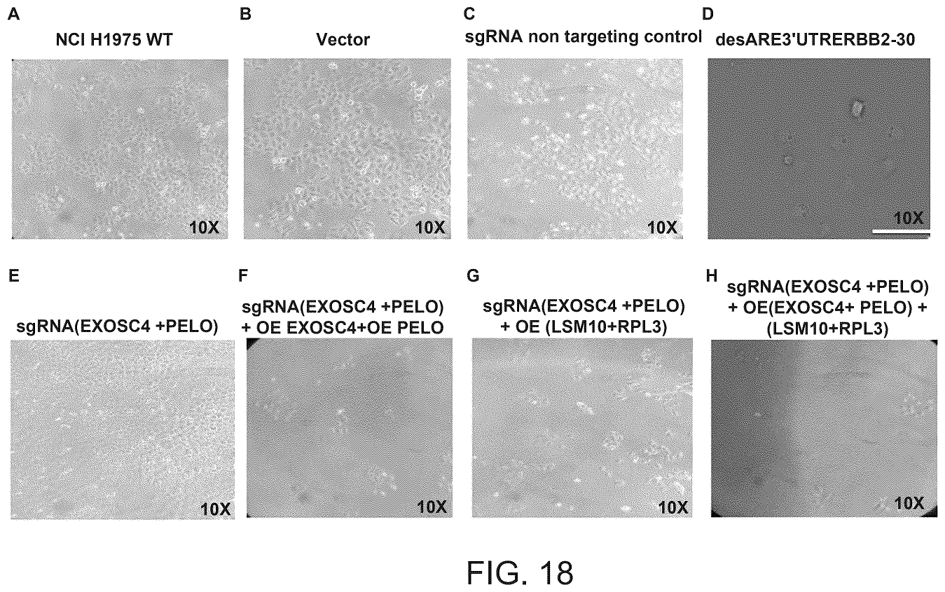

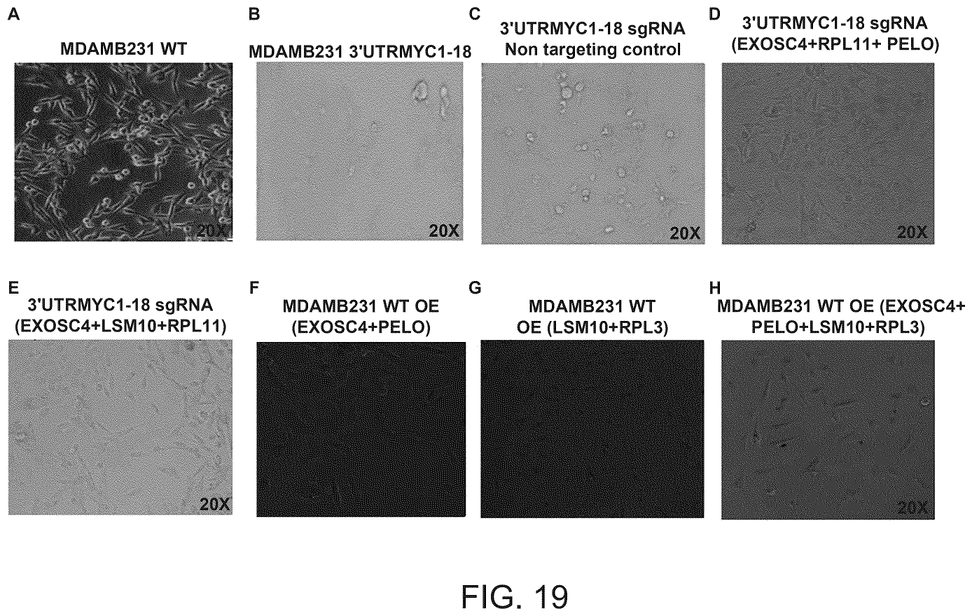

In sgRNA PELO and EXOSC4 knockout cells with overexpressed LSM10 and RPL3 also show reduction of cellular proliferation as seen in FIG. 18G. The levels of reduction are similar to that seen in FIG. 18F. The overexpression of all of PELO, EXOSC4, RPL3 and LSM10 completely abolishes the cell proliferation in the sgRNA PELO and EXOSC4 knockout cells treated with desARE3′UTRERBB2-30 (FIG. 18H). We found similar phenotypic effects in the MDAMB231 cells treated with 3′UTRMYC1-18 and CRISPR KO EXOSC4, PELO, LSM10 and RPL11 (FIG. 19A-E). This was not seen in MDAMB231 WT cells without the mRNA destabilizing drugs but with over expressed with EXOSC4, PELO, RPL3 and LSM10 (FIG. 19F, G, H). In conclusion, we document in detail the cellular phenotypes that accompanies the PELO, EXOSC4, and RPL3 switch of the ribosome fate in cancer cells treated with 3′UTR mRNA destabilizing drugs. We demonstrate that the 3′UTR mRNA destabilizing drugs work in concert with elevated levels of PELO, EXOSC4, and RPL3 to achieve degradation of the specific target mRNA.

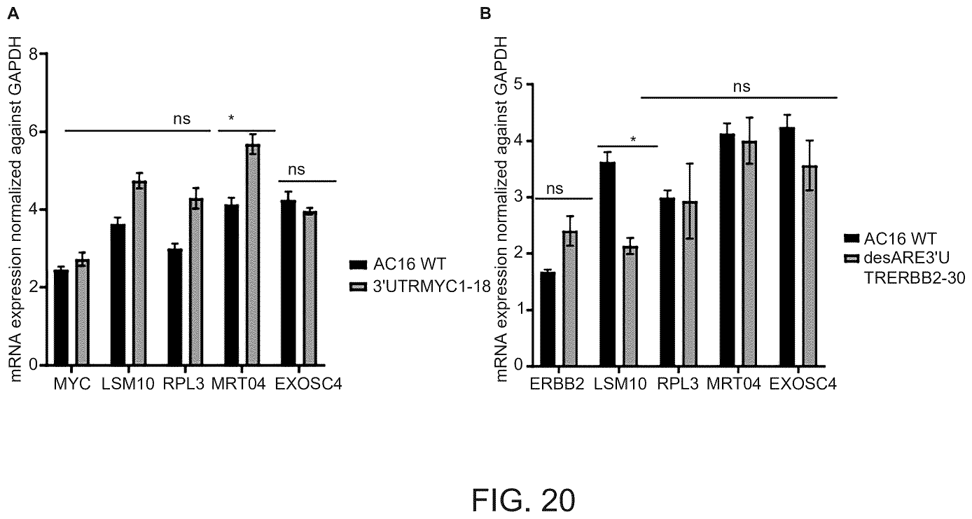

Non differential expressions of the PELO, EXOSC4 and RPL3 in the healthy cells explain why the target transcript are not degraded in them. We have defined mRNA destabilizing drug sequence specificity and how the PELO, EXOSC4 and RPL3 switches the ribosome from translating to degrading in the presence of the destabilized mRNA. Our next aim was to determine why the mRNA destabilizing drugs are safe for administration to healthy normal cells in vivo and in vitro. We investigated if the transcript of PELO, EXOSC4, RPL11, RPL3 and HBSIL are upregulated in the healthy cells AC16. We found that there is no differential expression of these transcripts in the healthy cells compared to the controls in the presence of the destabilized constructs (FIG. 20A-B). This explains the safety of the mRNA destabilizing drugs that we have observed in the multiple studies, including those involving epithelial cells and healthy cardiomyocytes. See, e.g., Examples 2-5 below.

### Example 1C: Discussion

Two major processes regulate ribosome quality assurance control; these include No-Go decay (NGD) and Nonsense mediated decay (NMD) (4-6, 7-8). The ribosomes trigger premature termination codon stalling at exon, in frame stalling, slow kinetics, disomes, colliding ribosomes, poly A stalling and 3′UTR stalling to induce the quality control machinery to remove and degrade the aberrant mRNA (9-13). Various conditions lead to this process, such as (i) non-optimal mRNA sequences, (ii) damaged RNA, (iii) stop codon on non-stop mRNA, (iv) translation inhibiting peptide or elongation inhibiting peptide, and (v) nascent polypeptide (4-13).

For a stalled ribosome in frame, HEL2 splits the trapped 80S ribosome which triggers the proteins NEMF and LTN to target the nascent polypeptide exiting the ribosome, with the c-terminal of such proteins serving a degron (9-13). For the ribosome stalled at the 3′UTR end, PELOTA (PELO)/HBSIL recognizes the stalled ribosome and triggers the ABCE1 to cleave the ribosome and thereafter recruiting the XRN1 and exosome to degrade the transcript (9-13).

We recently developed a novel mRNA overwriting technology based on the destabilization of the mRNA poly U stabilizing sequences on the 3′UTR of oncogenes such as c-MYC (16), ERBB2 (14-15) and TEAD1 (17) amongst others. With this technology, we specifically destabilized these oncogenic transcripts and triggered their degradation through DCP1A controlled nonsense mediated decay. However, it was not clear how the destabilized mRNA recognized their specific target mRNA, and what molecules mediate the switch from a proliferating cancer cell to a dying cancer cell once we treat the cells with the 3′UTR mRNA destabilizing drugs.

Our aim was to unravel how the destabilized mRNA recognizes the specific target mRNA. We used various plasmid systems which expressed various fragments of the target mRNA cDNA sequences. We treated each fragment of the target mRNA cDNA with the destabilized mRNA and observed that the destabilized 3′UTR mRNA recognized and inhibited the target mRNA expression in a sequence specific manner. We found in-frame recognition sites stronger than the 3′UTR and the 3′UTR recognition sites stronger than the 5′UTR, while the 5′UTR recognition sites lacked involvement. We found that multiple target site recognition sites were stronger than one site and both exon and intron target specific sites were recognized. Taken together, our results indicate that the EXOSC4 and PELO are the molecular regulators of ribosome fate switch in the presence of the destabilized mRNA and in various cellular states.

To summarize our findings: (1) Multiple in frame-mRNA recognition sites stalling for ribosome fate switch is stronger than on the 3′UTR site. We found that the ERBB2 mRNA destabilizing drugs achieved stronger down regulation of the ERBB2 (exon 2-6) (YFP-ErBB2 delta C990) than the ERBB2 (exon 26-27) (YFP-ErBB2 delta C776). Similarly, the MYC drug showed stronger downregulation of the MYC with the construct targeting the (exons 1-3) than the construct targeting the 132 bp sequence within the exon 2. This implies that multiple in-frame stalling as demonstrated shows stronger downregulation of the target transcript than one or two 3′UTR sites. (2) 3′UTR mRNA recognition sites for stalling ribosome fate switching are stronger than the 5′UTR. From the ERBB2 system, the mRNA destabilizing drug recognition of the target transcript on the ERBB2 exons 26-27 (YFP-ErBB2 delta C776) shows stronger down regulation of the target transcript than the 5′ first exon (YFP-ErBB2). This implies that stalling at the 3′UTR terminal exons is stronger than the 5. (3) 5′UTR sites are not involved. We found that the 5′ ERBB2 first exon was not targeted nor down regulated, which implies that the elements there are not involved. Our analysis of this region shows the absence of the ERBB2 response element CAGA. (4) Destabilized 3′UTR mRNA recognizes both the exon and intronic target specific site. The data from both the ERBB2 and MYC plasmid expression system show that the mRNA destabilizing drugs recognized specific elements on the ERBB2 introns 2-6 and intron 26 and the introns 1-2 on the MYC, respectively. While we have shown the presence of canonical and non-canonical E-box MYC sequences and the ERBB2 response elements as the target of the specific recognition. It is also plausible that the specific sequence recognition is mediated by direct RNA binding as mRNA-mRNA interaction and binding (20-23).

In sum, we report that the targets for the mRNA destabilizing drugs are sequence specific. We have validated the sequence specificity of the drugs on their target transcript and shown that the drugs recognize target mRNA specific sequence to down regulate them. We found that, once the drug is at the target site, the EXOSC4-PELO and RPL3 are recruited as the molecular regulators of the ribosome fate switch from translating to degrading and to mediate the target transcript degradation. This finding is important as it delineates a key function of how ribosome, a macromolecular machine, can perform its versatile protein production functions. Whilst the ribosome quality control proteins are well known, our findings here introduce a new paradigm which is that ribosome fate can be switched and regulated in various cellular states under the control of the destabilized 3′UTR mRNA therapy. This finding opens an unprecedented vista to control the basic process of life on a fine scale both in the normal cellular processes and diseases. The plasmid expression system used to define the sequence specificity of the mRNA destabilizing drugs offered insight and generalized conclusions to be reached.

### Example 2: Novel MYCN mRNA Destabilizing Drugs Therapeutically Inhibit Metastatic Rhabdomyosarcoma In Vivo with Significant Survival Outcomes

Overview: The metastatic aggressive rhabdomyosarcoma (RMS) is driven by the oncogenic MYCN amplification. There is no clinically approved drug to directly target MYCN. The MYCN oncogene belongs to the basic helix loop helix MYC superfamily and is implicated in many childhood cancers and adult cancers. We report the development of the novel 3′UTR MYCN mRNA destabilizing drugs, 3′UTRMYCNM1-14 and 3′UTRMYCNM1-18. The drugs destabilize the mRNA by replacement of polyU sequences on the MYCN 3′UTR thereby leading to the MYCN down regulation. In vivo, in the metastatic rhabdomyosarcoma, the novel MYCN mRNA destabilizing drugs are safe, well tolerated. We observed inhibition of primary tumors and metastasis, on-target down regulation of MYCN and MYOD1 and mutant p53, and very significant survival outcomes compared to the controls.

### Example 2A: Materials and Methods

Development of the 3′UTRMYCN mRNA destabilizing drugs: Briefly, we examined the 3′UTR of MYCN for the presence of the mRNA poly U stabilizing elements as we described previously (20a, 21a, 23a). We then engineered these stable elements into destabilizing forms with a minimal DCP1A promoter as the driver. This synthetic ensemble was checked for quality and passed g-block synthesis on IDT Inc and was synthesized as a DNA g-block from IDT Inc USA. We then amplified the construct by PCR as described (20a, 21a, 23a). The amplicon was gel extracted using the Qiagen kit (Cat ID:28704). We then set up blunt ligation as described (20a, 21a, 23a). Subsequently, we transformed competent E. co/i (NEB Cat ID: C2987H) with the ligation mixture and plated it on LB agar plate overnight incubated at 37° C. We picked colonies and miniprepped them and performed colony PCR with primers specific to the constructs. Sanger sequencing was performed on the colonies by Genewiz USA Inc.

Cell Culture: We obtained the neuroblastoma cells lines SKNBE2 from ATCC, Kelly and LAN1 from the DSMZ (Hannover, Germany) and the RD from ATCC. The cells were authenticated and tested negative for mycoplasma. We expanded the cells Kelly and LAN1 in RPMI 1640 media supplemented with 10% FBS and 5% antibiotic-antimycotic. The RD cells were grown in DMEM media supplemented with the 10% FBS and 5% antibiotic-antimycotic. The SKNBE2 cells were grown in the EMEM media supplemented with the 10% FBS and 5% antibiotic-antimycotic. The cells were grown to 80% confluency before use.

Dose dependent IC₅₀ determination and comparison with standard of care drugs: Assays were performed to determine the IC₅₀ of the novel MYCN mRNA destabilizing drugs. We carried out dose dependent titration of the drugs starting from 2.5 μg to 40 μg as well as standard of care drugs. This included olaparib, paclitaxel, cisplatin, actinomycin D, bevacizumab, cyclophosphamide and MYCi975. All of these drugs were obtained from Selleckchem (USA). We made a serial dilution of the drugs and seeded the cells at 5,000 cells per well in a 96 well plate. The cells were allowed to be attached for 24 hrs and then the drugs were added. We incubated the treated cells for 72 hrs and using the cell titre glo (Promega G7570), we read the viability and normalized it to the controls and the data curve were fitted on the drug dose response chart in the GraphPad Prism (USA). From this, the IC₅₀ were derived.

Quantitative reverse transcript PCR: We extracted the RNA using the Qiagen RNeasy kit (Cat No. 74104). The RNA was stored at −80° C. before use. To reverse transcribe the RNA, we used the Superscript IV reverse transcriptase kit (Cat No. 18090200) to make cDNA. We designed the qPCR primers targeting the exons of the MYCN, TERT, EZH2 and GAPDH as housekeeping gene controls. All the primers used are listed in Table 2A. The delta CT was used to normalize the transcript expression.

TABLE 2A
Primers utilized for qRT-PCR

| Primers | Forward | Reverse |
|---|---|---|
| MYCN | ACCACAAGGCCCTCAGTACCTC | TGACAGCCTTGGTGTTGGAGGA |
| | SEQ ID NO: 150 | SEQ ID NO: 151 |
| TERT | GCCGATTGTGAACATGGACTACG | GCTCGTAGTTGAGCACGCTGAA |
| | SEQ ID NO: 152 | SEQ ID NO: 153 |
| EZH2 | GCTCCACTGCCTTCTGAGT | AATAAAAGCGATGGCGATTGG |
| | SEQ ID NO: 154 | SEQ ID NO: 155 |
| GAPDH | GCCTCACTCCTTTTGCAGAC | AAGTGGTCGTTGAGGGCAAT |
| | SEQ ID NO: 156 | SEQ ID NO: 157 |

Migration assay: To study the MYCN mRNA destabilizing drug ability to inhibit cancer migration, we performed a wound healing assay. We seeded 10,000 cells in a 6 well plate for both the control and the treated cells. Once the cells grew to 70% confluency, we made a scratch wound and measured the wound distance starting from time point 0, and every 24 hrs until it closed. The data was plotted in GraphPad Prism (USA).

Iron oxide nanocage and 3′UTRMYCN mRNA destabilizing drug complexation: The iron oxide nanocage and the MYCN mRNA destabilizing drugs were complexed in a 1: 120-121 ratio and the conjugates were incubated over night at 4° C. before use. Subsequently, the drug conjugate was found to be stable both at room temperature, 4° C. or −20° C. until use.

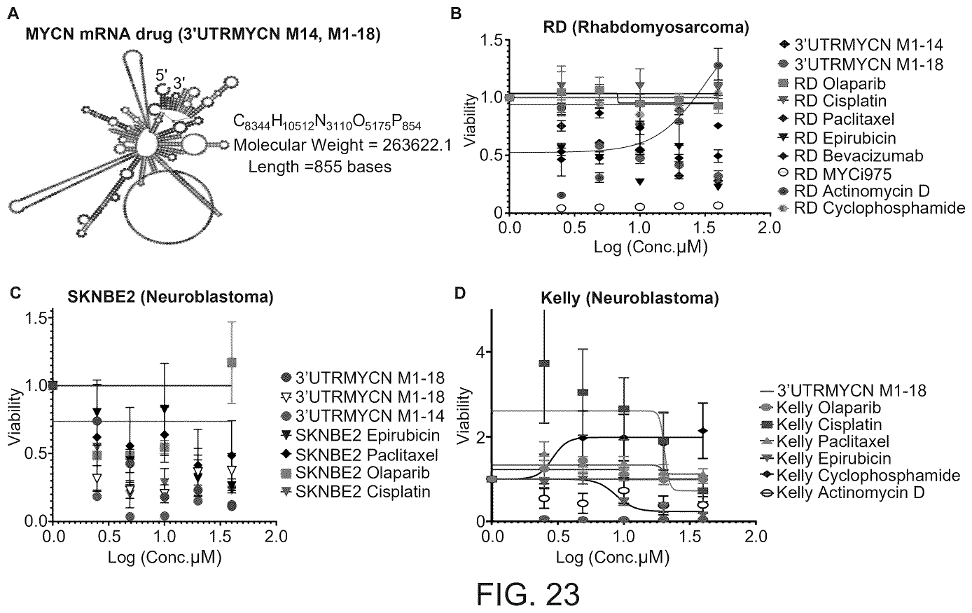

Animal study: We obtained IACUC institutional approval from the CUNY institutional board. We ordered the 10 NSG mice including 5 males and 5 females from the Jackson laboratory. Once received, we allowed the mice to acclimatize according to the protocol. We implanted 10 million RD cells into the leg muscle of the mice. After 26 days post implantation, the tumor was engrafted. We then randomized the animals into equal tumor volume and weight into three groups: 1) vector plus nanocage group (N=3) 2) 3′UTRMYCNM1-14+nanocage group (N=3) and 3) 3′UTRMYCNM1-18+nanocage group (N=4). Using the IC₅₀ dose of the drugs (FIG. 23; determined as 10 μg), this was administered to the animals intravenously 2× per week. On day 43, we collected the blood of the animals to assess for the safety profile. This included full blood count, electrolyte, liver, kidney, pancreatic and gall bladder function. We then dosed the animals 1× per week until day 50. We then took a 25-day dosing break. We recorded daily tumor volumes, weight, and body condition score. On the day 62, the 3′UTRMYCNM1-18 treated animals died. On day 74, the vector+nanocage treated group died. The 3′UTRMYCNM1-14 treated animals were alive up to 84 days, at which point the experiment ended.

Safety profile analysis: Assessments were used to determine the safety of the novel MYCN mRNA destabilizing drugs to the blood cells, electrolyte, kidney, pancreatic, and gall bladder function. We collected blood and serum and sent it to the Memorial Sloan Kettering Cancer Core Pathology laboratory. This laboratory ran the full blood count, lipid profile, liver enzymes, kidney function, and electrolyte analysis. The reference used was normal NSG mice. The analyst was blinded to the experimental details.

Necropsy: Upon death, we took the fresh carcasses and dissected them and collected the tumors, lungs, kidney, livers, and brain. We rinsed the lungs in 1×PBS and took the images of the fresh organs. Subsequently, we collected fresh tissues of tumors and organs and froze these at −80° C. All of the tissues were then placed in 4% paraformaldehyde until they were sent to the pathologist lab for embedding and H&E staining.

H&E staining of tumors and organs: For the H&E staining of all the tissues, the tumors, livers, lungs, and brain of the treated animals and controls were embedded and cut. H&E staining was performed by the Memorial Sloan Kettering Cancer Center Core Pathology lab. The pathologist was blinded to the experimental details. Images of the stained tissues were obtained using the EVOS FL microscope at 40× magnification. Quantification was performed in Image J (https://imagej.net/ij/) and in Graph Pad Prism (v10).

IHC staining of MYCN and MYOD1 of tumors and organs: We used IHC staining against MYCN and MYOD1 on the tumors and organs of the treated animals and the controls. For this, we employed the Abcam IHC protocol (https://www.abcam.com/en-us/technical-resources/protocols/ihc-with-samples-in-paraffin). We deparaffinized the tissue slides according to the protocol and performed enzymatic antigen retrieval using 1:1 trypsin concentrate and buffer. Washes were done with 1×TBST. Blocking was done with protein block (ab64212) for 1 hr. After this the washes were repeated. Next, we incubated the slides with primary antibodies against MYCN (Cell Signaling, Cat ID: (D4B2Y) or with MYOD1 (Cell Signaling: (D8G3) or p53 (Cell Signaling: p53 (7F5) rabbit mAb) overnight. Next day, the slides were washed with 1×TBST. The secondary antibody was added on the slide and incubated for 1 hr. Subsequently, we washed the slides with 1×TBST. To detect the signals, we used the DAB and concentrate and enhanced with enhancer. After this, we counter stained and added mounting media. The cover slip was added and the slides were sealed. Images were obtained on EVOS Fl at 40×. Target staining of MYCN and MYOD1 was quantified in Image J (https://imagej.net/ij/).

Statistical analysis: All experiments were performed in replicates and a minimum number of N=3. Paired T-test was used to determine statistical significance between treated groups and controls. Log Rank Mantel test was used to determine statistical significance between the in vivo treatment groups. The Kaplan Meier survival curve was used to determine survival difference between the various treatment groups and controls. All data was plotted with the GraphPad Prism (v.10).

### Example 2B: Results

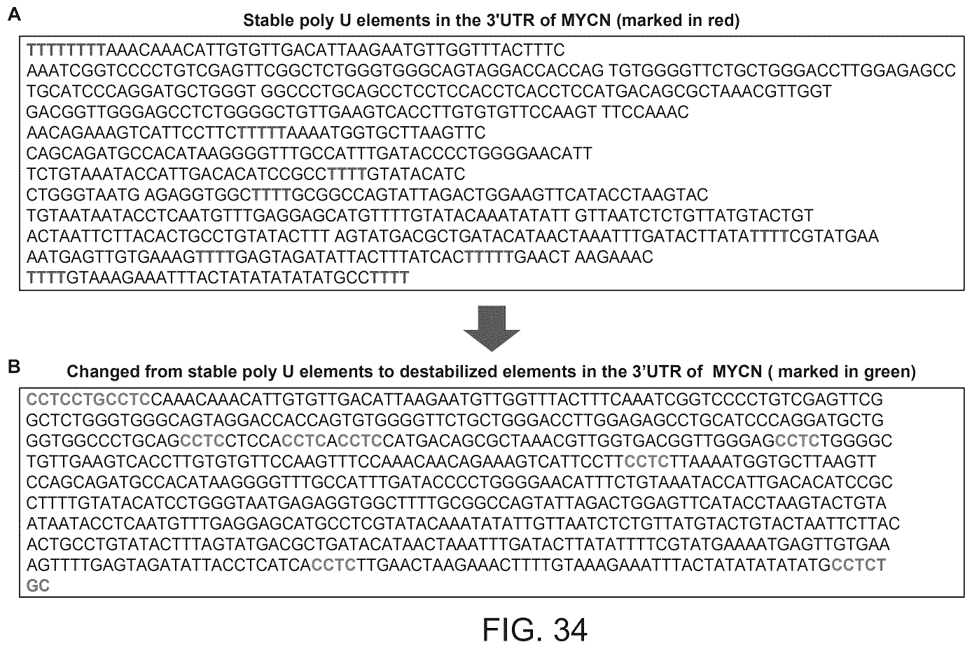

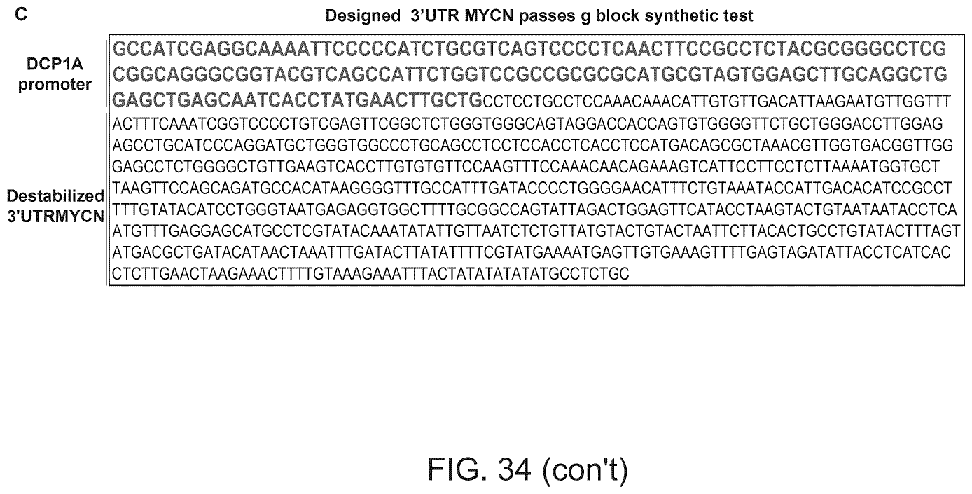

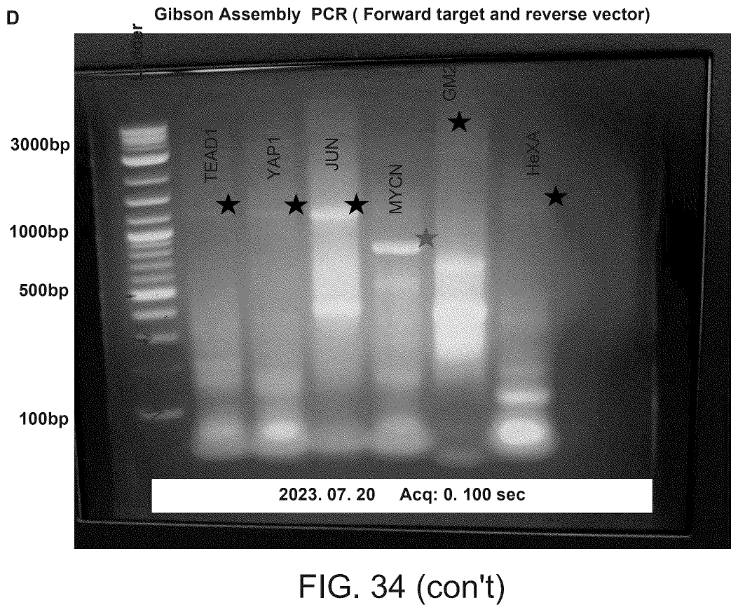

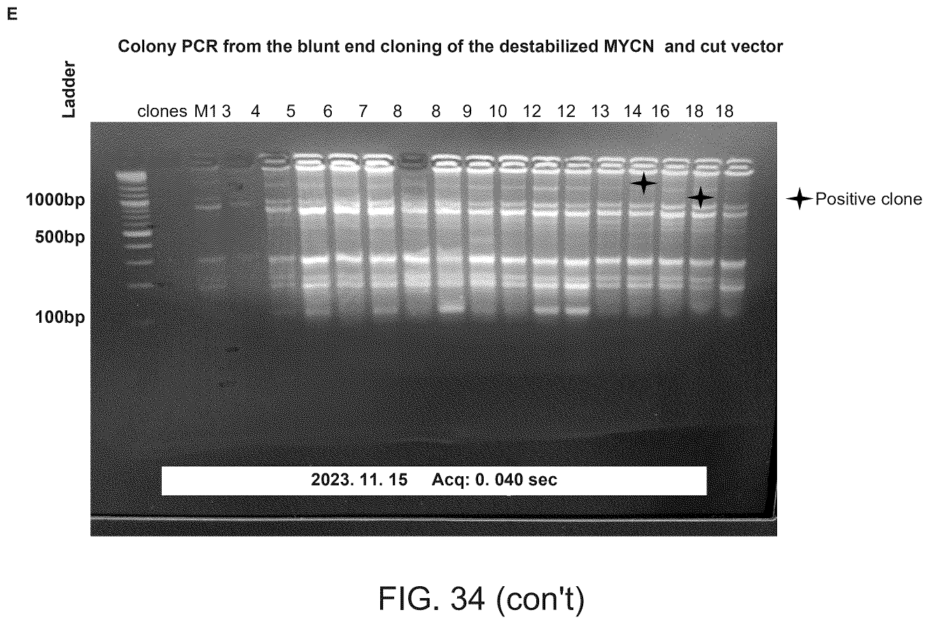

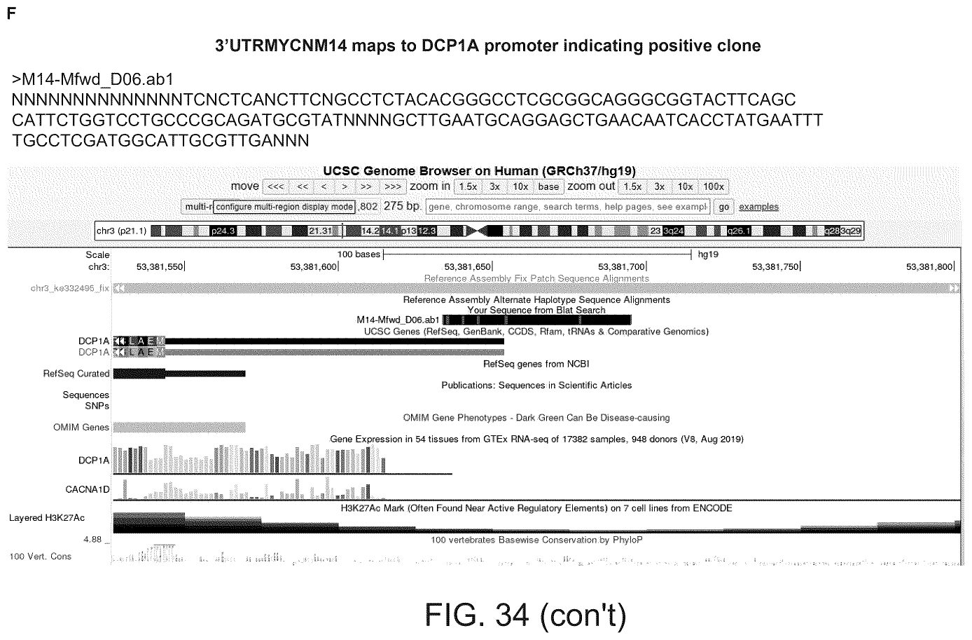

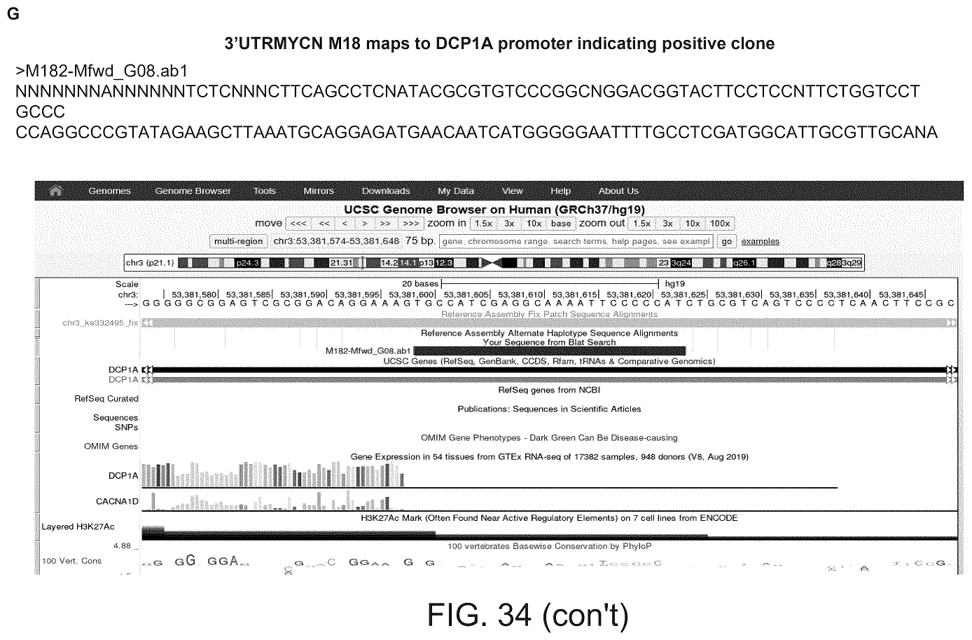

Development of novel MYCN mRNA destabilizing drugs and the determination of the IC₅₀ in metastatic neuroblastoma and rhabdomyosarcoma. To develop the MYCN mRNA destabilizing drugs, we evaluated the 3′UTR of MYCN and assessed for the presence of the mRNA stabilizing elements on the MYCN 3′UTR. We found these elements (FIG. 34A) and then we changed those sequences to the destabilizing form driven by the minimal DCP1A promoter (FIG. 34B, C) in order to trigger the MYCN mRNA specific degradation once it is destabilized on the 3′UTR. The synthetic engineered destabilized 3′UTR MYCN was obtained from IDT and amplified by PCR (FIG. 34C) and then cloned into the vector (pLenti-CMVSP6-nEGFP-SV40-PURO (Addgene: #138364) by blunt ligation. The transformation of competent E. coli yielded 17 clones of which the clones M1-14 and M1-18 (3′UTRMYCNM1-14, 3′UTRMYCNM1-18) were positive. These mapped back to the minimal promoter of the DCP1A in the synthetic 3′UTRMYCN construct, confirming that they are positive clones (FIG. 34D). The RNA secondary structure, molecular formula, molecular weight, and length of nucleic acid bases of the MYCN mRNA destabilizing drugs are shown in FIG. 23A.

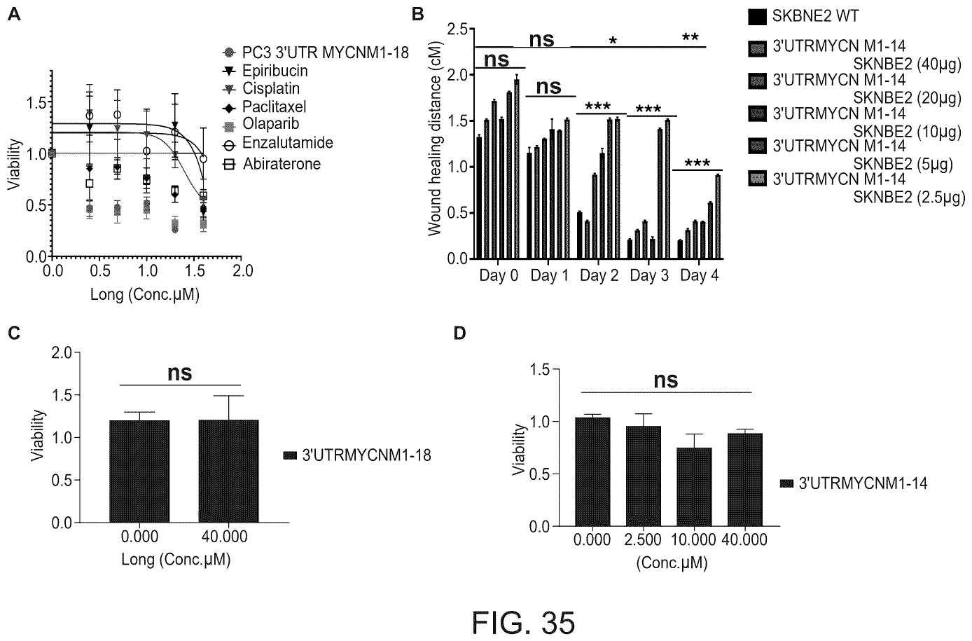

We sought to determine the therapeutic IC₅₀ dose, in vitro, for the MYCN mRNA destabilizing drugs (3′UTRMYCNM1-14 and M1-18). We performed drug dose dependent titration of the drugs in a head-to-head comparison with the standard of care drugs (SOC) in 3 MYCN amplified childhood cancers. These included: RD-rhabdomyosarcoma, aggressive chemo and radiotherapy resistant neuroblastoma cell lines (SKNBE2 and Kelly) (FIG. 23B-D) and androgen independent prostate cancer cells PC3 (FIG. 35A). We found that the MYCN mRNA destabilizing drugs show superior IC₅₀ (10 μM) compared to the IC₅₀ of olaparib, actinomycin D, cisplatin, cyclophosphamide, bevacizumab and MYCi975 in the RD cells (FIG. 23B). We found that the IC₅₀ of the epirubicin and paclitaxel is within the same range as MYCN mRNA destabilizing drugs in RD cells. In the lethal neuroblastoma cells, SKNBE2 (FIG. 23C) and Kelly (FIG. 23D), the MYCN mRNA destabilizing drugs achieved a superior IC₅₀ (5-10 μM) compared to the IC₅₀ of the olaparib, cisplatin, paclitaxel, cyclophosphamide, and actinomycin D. The epirubicin IC₅₀ was comparative to the MYCN mRNA destabilizing drugs in the SKNBE2 than in the Kelly cells (FIG. 23C-D). In the PC3 cells, 3′UTRMYCNM1-18 shows similar performance as olaparib but superior performance compared to epirubicin, paclitaxel, cisplatin, enzalutamide and abiraterone (FIG. 35A).

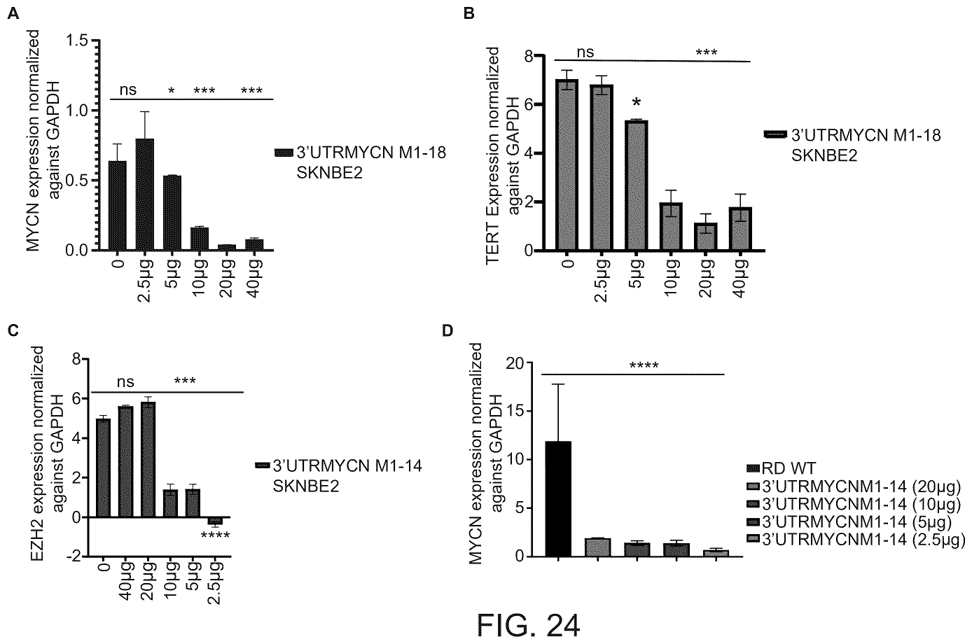

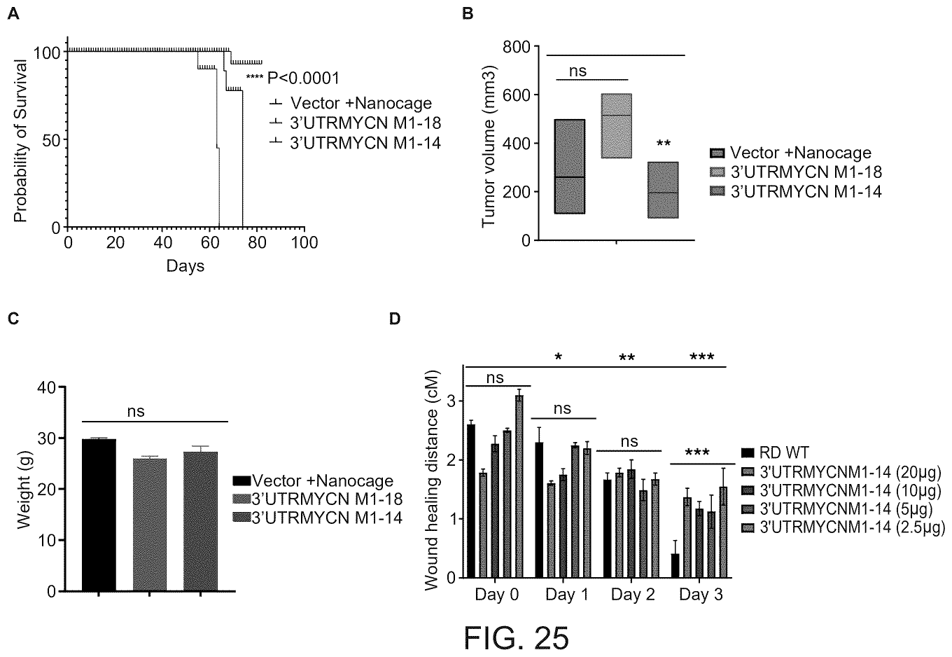

The novel MYCN mRNA destabilizing drugs downregulate the MYCN mRNA transcript and its interactome in a dose dependent manner. We sought to validate the on-target specificity of the MYCN mRNA destabilizing drugs on the MYCN transcript. We evaluated the MYCN expression on the childhood cancer cells treated in dose dependent manner with the MYCN mRNA destabilizing drugs. We found that the mRNA destabilizing drugs downregulated the MYCN mRNA expression in the SKNBE2 cells in a dose dependent manner (FIG. 24A). It is known that in the MYCN amplified neuroblastoma cells, TERT expression is upregulated (10a-11a). We investigated whether the expression of TERT is downregulated once the MYCN is downregulated. Indeed, we found that the dose dependent downregulation of TERT in the MYCN amplified cells treated with the MYCN mRNA destabilizing drugs (FIG. 24B). Others have also shown that polycomb repressor complex protein EZH2 is upregulated in the MYCN amplified neuroblastoma cells. Evidence has pointed to the indirect inhibition of the MYCN expression via the downregulation of EZH2 with the EZH2 inhibitors (8a, 9a, 17a). We performed experiments to determine if EZH2 expression is downregulated upon the destabilization and downregulation of the MYCN. We found that EZH2 expression is downregulated upon the MYCN destabilization (FIG. 24C). Finally, we found that MYCN mRNA destabilizing drugs downregulated MYCN in the rhabdomyosarcoma cells (FIG. 24D). Taken together, these results demonstrate that the novel MYCN mRNA destabilizing drugs target and destabilize MYCN and its interactome to achieve MYCN down regulation.

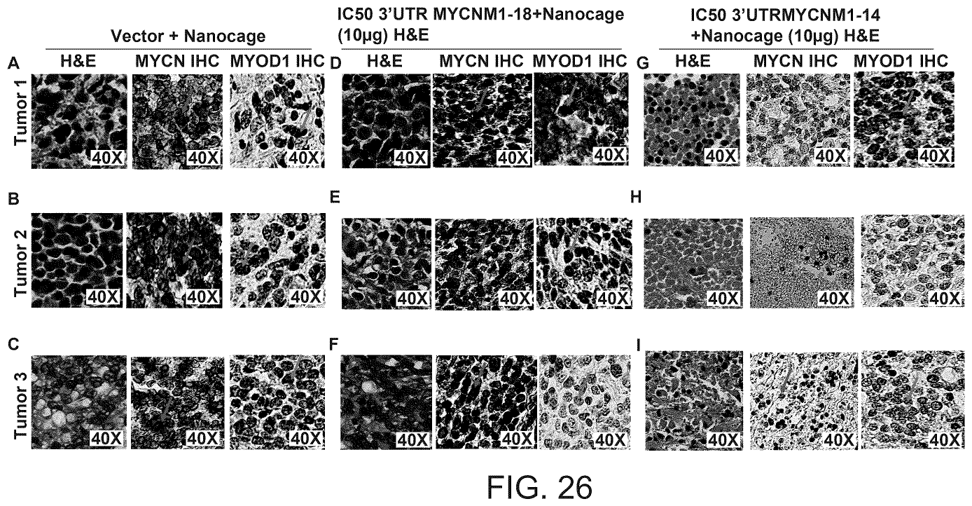

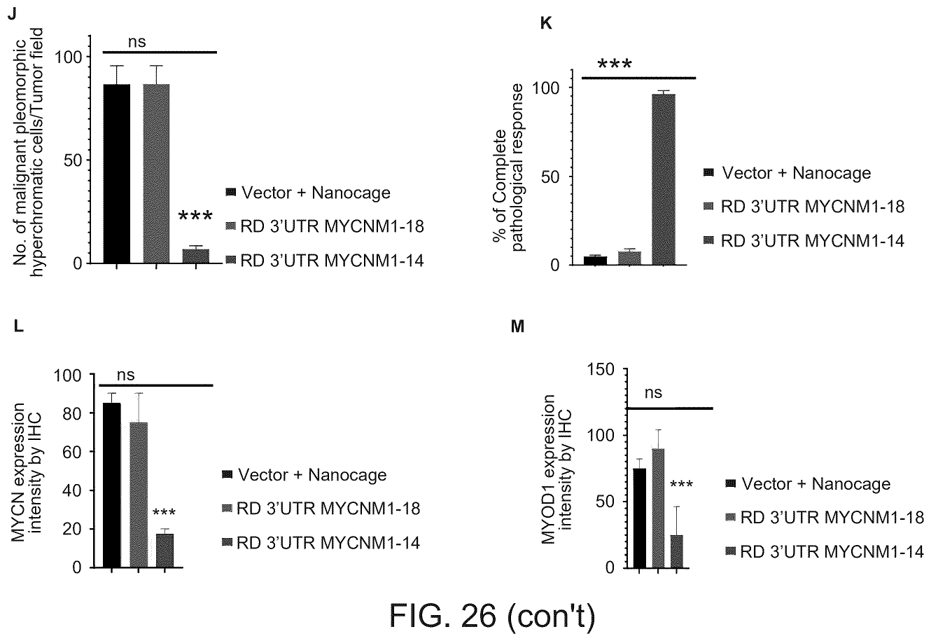

Next, we investigated whether MYCN mRNA destabilizing drugs inhibited the MYCN that is driven in children's cancers. We treated the lethal neuroblastoma and rhabdomyosarcoma cells with the MYCN mRNA destabilizing drugs in a dose dependent manner and then performed a wound healing assay in the treated cells and controls. We found that the drugs impaired the migration ability of the cancer cells in the neuroblastoma and rhabdomyosarcoma cells (FIG. 26C, FIG. 35B). This data supports the anti-cancer activity of the novel MYCN mRNA destabilizing drugs. In normal cardiomyocytes, such as AC16, the drugs did not impair the viability of the cells (FIG. 35C-D). This points to the safety of MYCN mRNA destabilizing drugs in healthy cells.

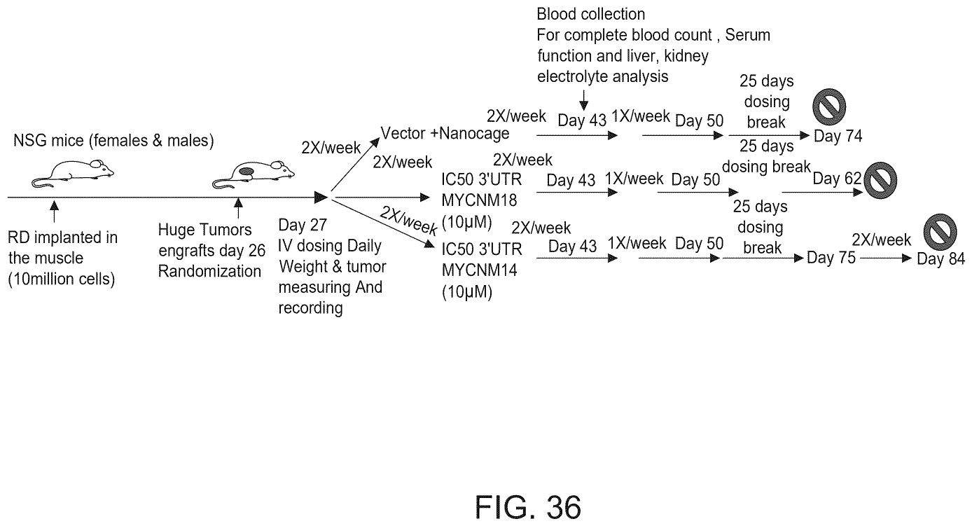

MYCN mRNA destabilizing drugs achieved in vitro therapeutic inhibition of tumors and lung metastasis with significant survival outcomes. Our next aim was to prove the therapeutic efficacy of the MYCN mRNA destabilizing drugs in vivo. We acquired IACUC approval from the institutional review board and obtained 9 NSG mice from the Jackson laboratory (5 males and 4 females of the same age). Once the animals arrived, we allowed them to acclimate according to the protocol. We implanted 10 million RD cells orthotopically in the thigh muscles of the mice (FIG. 36A). The tumors engrafted after 26 days. The same day the animals were randomized into groups according to tumor volume and weight. These included: 1) vector+nanocage (N=3), 2) IC₅₀ 3′UTRMYCNM1-18+10-nanocage N=4, 3) IC₅₀ 3′UTRMYCNM1-14+IO-nanocage, N=3. On day 27, we began intravenous dosing (IV) of the animals 2× per week. We recorded daily tumor volume, weight measurements, and body condition (FIG. 36A). After 2 weeks, we collected blood for complete blood count, serum, liver, electrolyte, and kidney function analysis (FIG. 36A). We continued dosing 1λ/week up to 50 days. After 50 days, we took a dosing break for 25 days. The 3′UTRMYCNM1-18 treated mice died on day 62. On day 74 the vector+nanocage group treated mice died and on day 84, the 3′UTRMYCNM1-14 treated group died (FIG. 36A). We found that 3′UTRMYCNM1-14 treatment achieved very significant survival outcomes (***P<0.0001) as compared to the control (FIG. 26A). Treatment with 3′UTRMYCNM1-14 yielded 10 days survival benefits as compared to treatment with vector. This equates to 400 days in human life, given that 1 mouse day is equivalent to 40 human days. Therefore, 3′UTRMYCNM1-14 treatment achieves a human survival difference of 1 yr and 1 month in metastatic rhabdomyosarcoma cancer (see, e.g., FIG. 26A) with significant tumor volume reduction (FIG. 26B).

The MYCN mRNA destabilizing drug achieved on target down regulation of MYCN in vivo in the tumors and the lungs. To validate in vivo that the MYCN mRNA destabilizing drug achieved on target downregulation of the MYCN, we performed H&E staining of the tumors, lungs, livers, and brain as well as MYCN IHC staining of the same tissues. We found that the MYCN mRNA destabilizing drug, 3′UTRMYCNM1-14, achieved greater than 90% degrading and lysis of the malignant pleomorphic hyperchromatic cells per tumor field (FIG. 26A-K) as compared to the controls. The mRNA destabilizing drug 3′UTRMYCNM1-14 also achieved more than 90% complete pathological response compared to the controls (FIG. 26G-I). Taken together, these results show that 3′UTRMYCNM1-14 is highly efficacious in vivo.

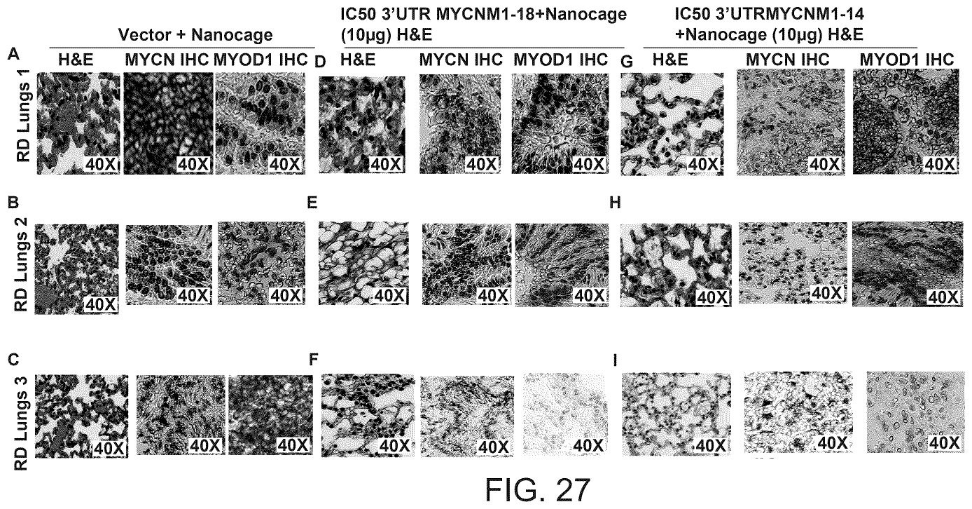

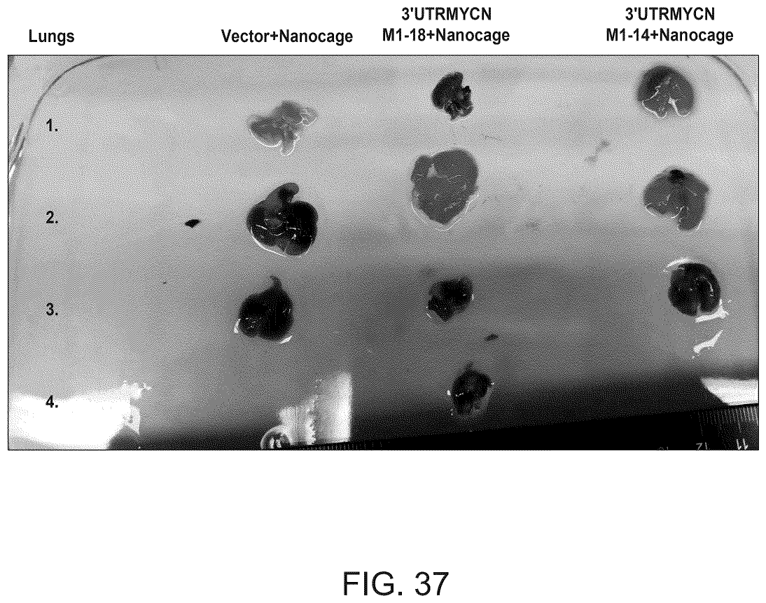

Since aggressive rhabdomyosarcoma metastasizes to the lungs, we investigated if the MYCN mRNA destabilizing drugs inhibited metastasis to the lungs. First, we examined the gross pathology of the lungs obtained from the different treatment groups. We found that the 2 out of 3 lungs in the 3′UTRMYCNM1-14 treated groups were healthy compared to 1 out of 3 in the 3′UTRMYCNM1-18 and the vector+nanocage treated groups (FIG. 37A). To validate this, we performed H&E staining of the lung tissues from the treated and controls. We found that 3′UTRMYCNM1-14 and M1-18 inhibited hemorrhagic cells in the lung parenchyma up to 90% compared to the controls (FIG. 27A-I). These results were quantified as shown in (FIG. 27J). 3′UTRMYCNM1-14 treatment achieved ~60% inhibition and degradation of the malignant hyperchromatic pleomorphic cells in the lung tissues (FIG. 27A-I). These results were quantified as shown in (FIG. 27K). Finally, we assessed the preservation of lung parenchyma. We found that 3′UTRMYCNM1-14 treatment achieved greater than 75% of the parenchyma preservation (FIG. 27G-I). These results were quantified as shown in (FIG. 27L). In summary, the 3′UTRMYCNM1-14 mRNA destabilizing drug is effective in inhibiting lung metastasis.

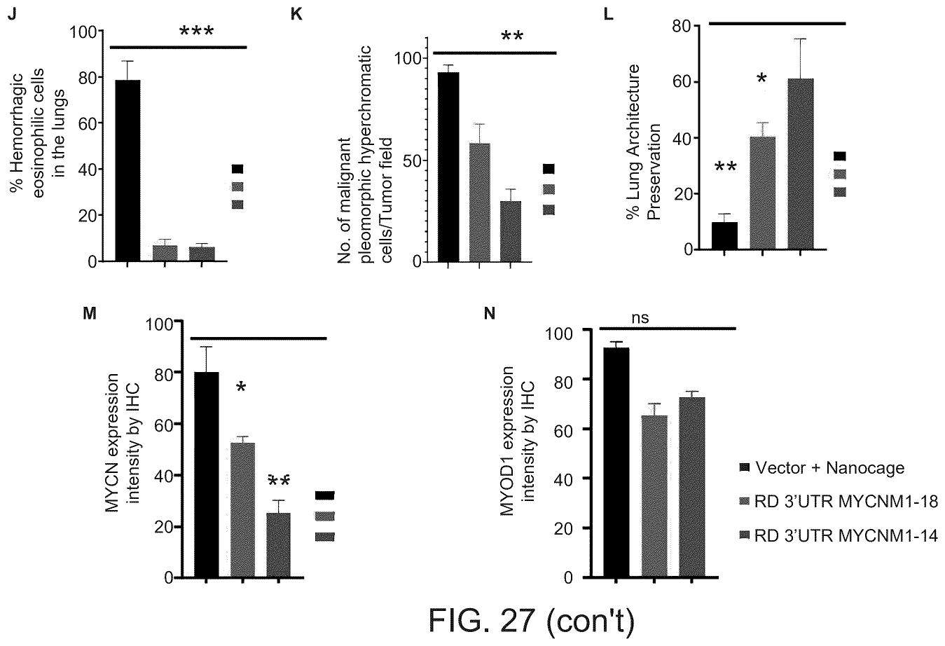

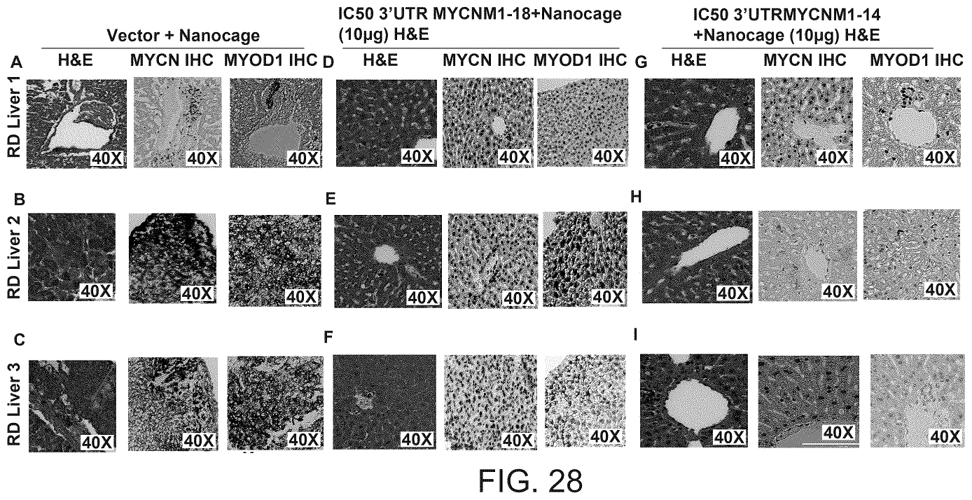

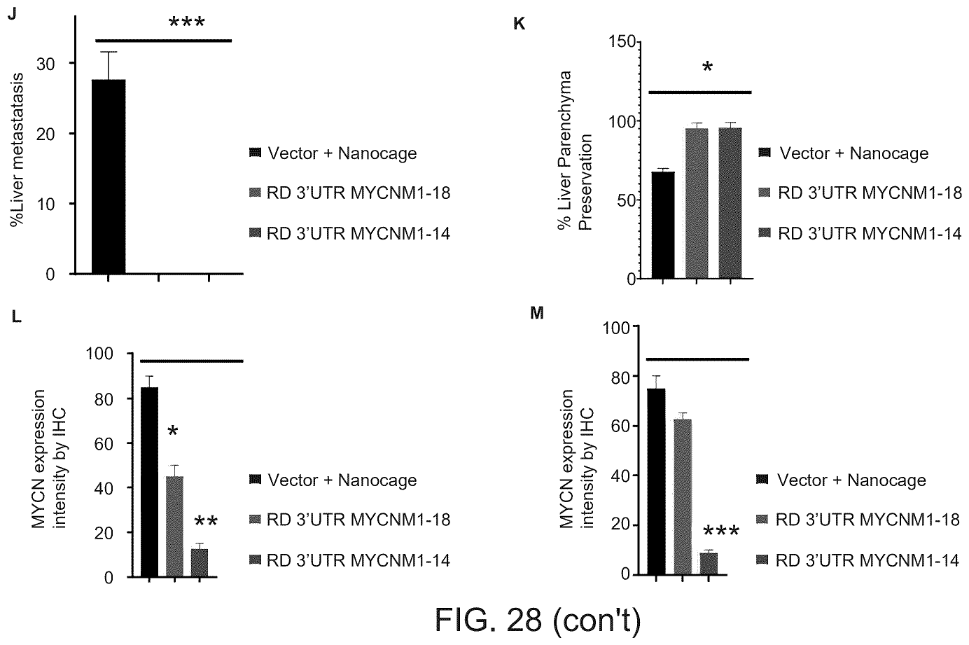

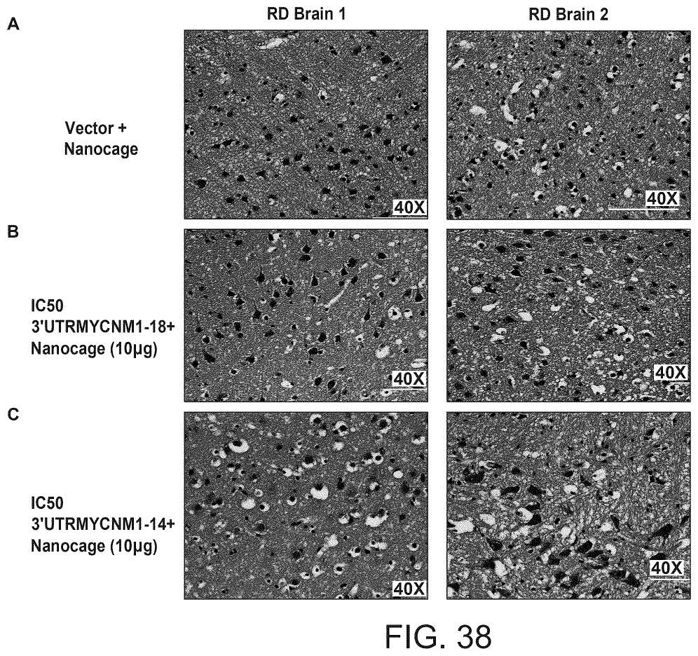

To assess the drug's efficacy in inhibiting liver metastasis, we performed H&E staining of liver tissue. We found that 1 out of 3 livers had metastasis in the vector+nanocage treated group (FIG. 28A-C). These results were quantified as shown in FIG. 28J. The 3′UTRMYCNM1-14 and M1-18 mRNA treated groups showed no liver metastases (FIG. 28D-I). Moreover, we found that the MYCN mRNA destabilizing drug treated mice had their liver parenchyma and architecture preserved compared to the controls (FIG. 28K). Lastly, we assessed brain metastasis by H&E staining of brain tissues from the treated and control groups. We found no evidence of brain metastasis (FIG. 38A) in the treated and the control groups. This suggested that the rhabdomyosarcoma did not metastasize to the brain.

The MYCN mRNA destabilizing drug achieved therapeutic efficacy and the inhibition of metastasis and the on-target downregulation of the MYCN and MYOD1 and mutant p53 protein. Our next aim was to validate that the MYCN mRNA destabilizing drugs were on-target and that the drugs acted to down regulate MYCN protein. We performed IHC staining against MYCN on the tumor samples treated with: 1) the vector+nanocage, 2) 3′UTRMYCNM1-18+nanocage and 3) 3′UTRMYCNM1-14+nanocage (FIG. 26A-I). We found high nuclear expression of MYCN in the tumors treated with vector+nanocage and 3′UTRMYCNM-18 (FIG. 26A-F). The tumors treated with the MYCN mRNA destabilizing drug, 3′UTRMYCNM1-14, significantly down regulated and inhibited the MYCN protein (FIG. 26G-K) with complete and near complete pathological response. This confirms that the MYCN 3′UTR mRNA destabilizing drug is on-target and specific in achieving its therapeutic efficacy.

MYOD1 is a marker of aggressive RMS and indicates the switch of muscle program from normal differentiation to cancer cells proliferation. We stained for MYOD1 expression, and we found a very high-level of MYOD1 in the nucleus of the tumors from the vector+nanocage treated group (3/3) (FIG. 26A-C) and the 3′UTRMYCNM1-18 treated group (3/3) (FIG. 26D-F). Conversely, MYOD1 expression was significantly downregulated in the tumors (2/3) treated with 3′UTRMYCNM1-14 (FIG. 26G-I). This indicates that the mRNA destabilizing drug downregulated MYCN and its direct interactor MYOD1 in RMS.

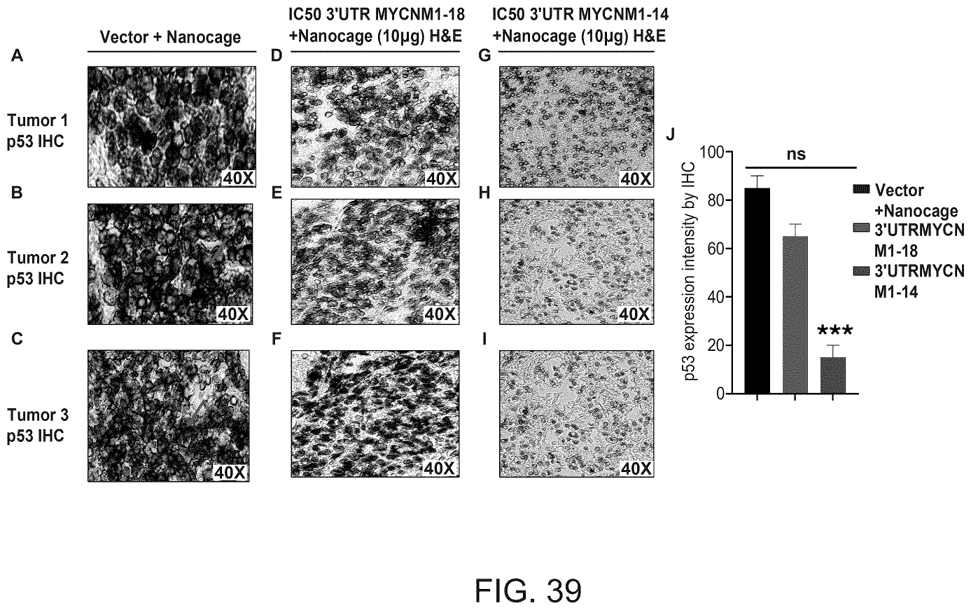

The mutant p53 is also implicated in the aggressive RMS. We stained the tumors with antibody against p53 and found markedly elevated levels of the p53 proteins in the vector+nanocage treated group and the 3′UTRMYCNM1-18+nanocage treated group (FIG. 39A-C, D-F). In contrast, the 3′UTRMYCNM1-14+nanocage treated group showed down regulation of the p53 protein (FIG. 39G-I).

Because RMS metastasizes to the lungs, we sought to assess whether the MYCN mRNA destabilizing drugs inhibited MYCN in the lungs and also inhibited lung metastasis. We stained the lungs from the vector+nanocage treated group, the 3′UTRMYCNM1-18+nanocage treated group and 3′UTRMYCNM1-14+nanocage treated group for MYCN expression (FIG. 27A-I). We found that MYCN expression is markedly elevated in the vector+nanocage treated group (FIG. 27A-C). MYCN shows moderate expression in the 3′UTRMYCNM1-18 treated group (FIG. 27D-F) and the lowest expression in the 3′UTRMYCNM1-14 treated group (FIG. 27G-I). This confirms the on-target specificity of the mRNA destabilizing drugs in targeting the MYCN to inhibit lung metastasis.

Next, we investigated if MYOD1 expression was down regulated in the lungs. We found no significant change between the controls and the treated group (FIG. 27A-I). Lastly, we investigated if the novel mRNA destabilizing drug down regulated MYCN in the liver to inhibit liver metastasis. We found a gross metastatic lesion in 1/3 livers from the vector+nanocage treated group but none in the 3′UTRMYCNM1-18 or M1-14 treated groups (FIG. 28J). We found very high expression of MYCN in the liver of the vector+nanocage treated group (2/3) (FIG. 28A-C). The 3′UTRMYCNM1-18 treated group showed moderately upregulated MYCN in the liver (3/3) (FIG. 28D-F). By comparison, 3′UTRMYCNM1-14 treatment significantly down regulated the MYCN in the liver to inhibit metastasis to the liver (3/3) (FIG. 28G-I).

We further investigated MYOD1 downregulation in the control versus the MYCN mRNA destabilizing drug treated liver. We found a markedly elevated levels of the MYOD1 in the livers of the vector+nanocage treatment group (2/3) and the 3′UTRMYCNM1-18 treatment group (2/3). On the other hand, 3′UTRMYCNM1-14+nanocage treatment significantly inhibited the MYOD1 expression in the liver (3/3) (FIG. 28G-I, K-M). Taken together, these results suggest that there is on-target MYCN mRNA inhibition by the MYCN mRNA destabilizing drug, MYCN, MYOD1, and p53 downregulation as well as inhibition of distant liver and lung metastasis.

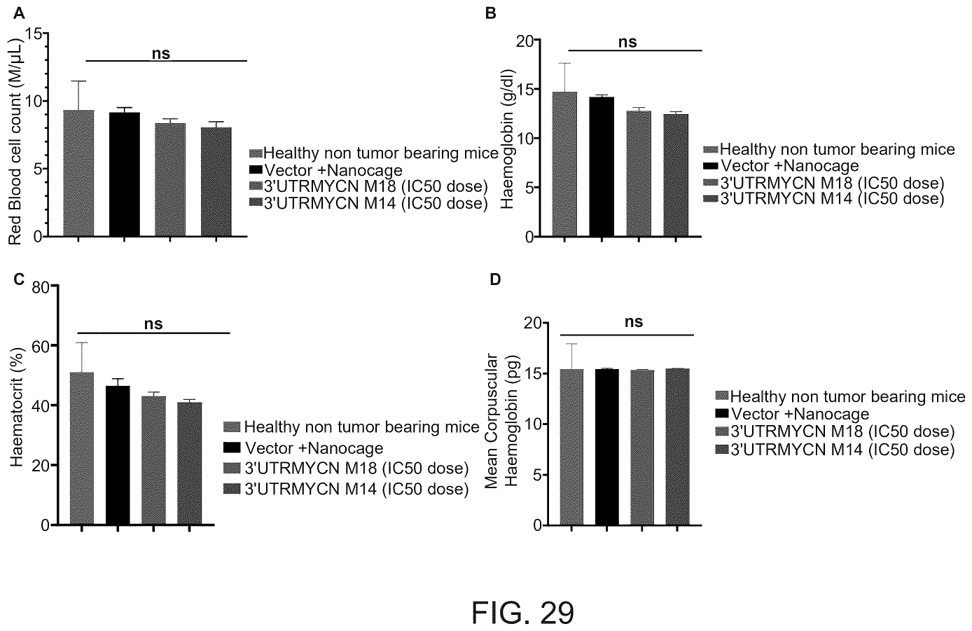

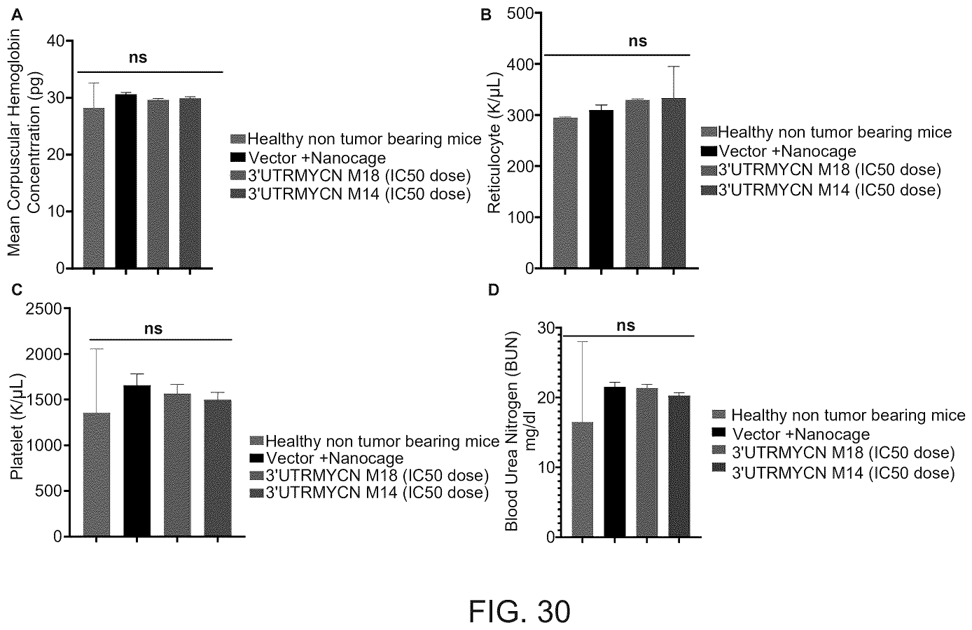

The MYCN mRNA destabilizing drugs have a positive safety profile for blood, electrolyte, kidney, liver, and pancreatic function. Our next aim was to demonstrate the safety of the drugs in vivo. We collected blood at 43 days into the in vivo experiment (FIG. 36A). We assayed the safety of MYCN treatment as compared to the controls of vector+nanocage treatment and healthy untreated mice (Tables 2B-2C). We found that the MYCN mRNA destabilizing drugs did not reduce the red blood cell count (FIG. 29A), the hemoglobin (FIG. 29B), the hematocrit (FIG. 29C), or the mean corpuscular hemoglobin (FIG. 29D). We also found no change in the mean corpuscular hemoglobin concentration (FIG. 30A), the reticulocyte (FIG. 30B), or the platelet (FIG. 30C) counts.

TABLE 2B
Complete blood count

| Acc. No. | 24-6183-1 | 24-6183-2 | 24-6183-13 | 24-6183-14 | 24-6183-15 | 24-6183-16 | 24-6183-19 | 24-6183-20 | 24-6183-21 |
|---|---|---|---|---|---|---|---|---|---|
| Sample | Vector + | Vector + | Vector + | Vector + | IC₅₀ dose | IC₅₀ dose | IC₅₀ dose | IC₅₀ dose | IC₅₀ dose |
| ID: | Nanocage | Nanocage | Nanocage | Nanocage | 3′UTRMY | 3′UTRMY | 3′UTRMY | 3′UTRMY | 3′UTRMY |
| | #1 | #2 | #3 | #4 | CN M1-18 | CN M1-18 | CN M1-14 | CN M1-14 | CN M1-14 |
| | | | | | #1 | #2 | #1 | #2 | #3 |
| Sex: | F | F | M | M | F | M | M | F | M |
| Age: | | | | | | | | | |

RBC & Platelet Indices

| | | | | | | | | | | Reference |
|---|---|---|---|---|---|---|---|---|---|---|
| Test name | | | | | | | | | | range |
| RBC (M/uL) | 8.89 | 9.41 | 9.13 | 8.22 | 8.13 | 8.59 | 8.34 | 7.75 | 7.98 | 7.84-10.84 |
| HGB (g/dL) | 13.80 | 14.60 | 14.10 | 12.60 | 12.40 | 13.10 | 12.90 | 12.00 | 12.40 | 11.80-17.60 |
| HCT (%) | 44.80 | 48.20 | 46.30 | 42.20 | 42.20 | 44.00 | 42.80 | 40.40 | 40.90 | 44.10-58.30 |
| MCV (fL) | 50.40 | 51.20 | 50.70 | 51.30 | 51.90 | 51.20 | 51.30 | 52.10 | 51.30 | 51.10-58.60 |
| MCH (pg) | 15.50 | 15.50 | 15.40 | 15.30 | 15.30 | 15.30 | 15.50 | 15.50 | 15.50 | 13.70-17.20 |
| MCHC (g/dL) | 30.80 | 30.30 | 30.50 | 29.90 | 29.40 | 29.80 | 30.10 | 29.70 | 30.30 | 25.10-31.30 |
| RDW-SD (fL) | 27.60 | 28.90 | 30.40 | 31.10 | 29.70 | 30.20 | 28.20 | 26.90 | 28.40 | — |
| RDW-CV (%) | 16.80 | 17.30 | 18.20 | 17.80 | 16.90 | 17.40 | 16.90 | 14.40 | 16.30 | 17.30-20.30 |
| RET# (K/uL) | 317.40 | 303.00 | 359.70 | 373.20 | 328.50 | 331.60 | 377.00 | 290.60 | 370.30 | 294.00-444.00 |
| RET (%) | 3.57 | 3.22 | 3.94 | 4.54 | 4.04 | 3.86 | 4.52 | 3.75 | 4.64 | 2.56-4.56 |
| PLT (K/uL) | 1670.00 | 1432.00 | 1865.00 | 1649.00 | 1667.00 | 1462.00 | 1488.00 | 1363.00 | 1641.00 | 651.00-2055.00 |
| PDW (fL) | 6.40 | 7.20 | 6.60 | 6.50 | 6.40 | 6.70 | 6.40 | 6.30 | 6.70 | — |
| MPV (fL) | 6.70 | 7.00 | 6.80 | 6.80 | 6.70 | 6.80 | 6.70 | 6.70 | 7.00 | 4.20-6.30 |

Automated Differentials

| | | | | | | | | | | Reference |
|---|---|---|---|---|---|---|---|---|---|---|
| Test Name | | | | | | | | | | range |
| WBC# (K/uL) | 1.63 | 1.89 | 1.59 | 1.81 | 0.84 | 1.48 | 1.19 | 1.04 | 1.32 | 0.94-4.68 |
| NEUT# (K/uL) | 1.15 | 1.46 | 0.93 | 1.18 | 0.55 | 0.68 | 0.79 | 0.71 | 0.96 | 0.54-3.16 |
| LYMPH# (K/uL) | 0.31 | 0.27 | 0.41 | 0.35 | 0.13 | 0.46 | 0.15 | 0.19 | 0.23 | 0.23-1.56 |
| MONO# (K/uL) | 0.13 | 0.11 | 0.22 | 0.25 | 0.15 | 0.31 | 0.23 | 0.13 | 0.12 | 0.03-0.26 |
| EO# (K/uL) | 0.03 | 0.04 | 0.03 | 0.03 | 0.01 | 0.03 | 0.02 | 0.01 | 0.01 | 0.00-0.39 |
| BASO# (K/uL) | 0.01 | 0.01 | 0.00 | 0.00 | 0.00 | 0.00 | 0.00 | 0.00 | 0.00 | 0.00-0.15 |
| NEUT (%) | 70.60 | 77.30 | 58.50 | 65.20 | 65.40 | 46.00 | 66.40 | 68.20 | 72.70 | 44.21-79.92 |
| LYMPH (%) | 19.00 | 14.30 | 25.80 | 19.30 | 15.50 | 31.10 | 12.60 | 18.30 | 17.40 | 13.51-42.61 |
| MONO (%) | 8.00 | 5.80 | 13.80 | 13.80 | 17.90 | 20.90 | 19.30 | 12.50 | 9.10 | 1.71-10.93 |
| EO (%) | 1.80 | 2.10 | 1.90 | 1.70 | 1.20 | 2.00 | 1.70 | 1.00 | 0.80 | 0.29-10.32 |
| BASO (%) | 0.60 | 0.50 | 0.00 | 0.00 | 0.00 | 0.00 | 0.00 | 0.00 | 0.00 | 0.00-3.55 |

TABLE 2C
Chemistry panel

| Acc. No. *same as Table 2B | * | * | * | * | * | * | * | * | * | Reference range |
|---|---|---|---|---|---|---|---|---|---|---|
| Sample ID **same as Table 2B | ** | ** | ** | ** | ** | ** | ** | ** | ** | |
| Sex: | F | F | M | M | F | M | M | F | M | |
| Age: | | | | | | | | | | |

| | | | | | | | | | | |
|---|---|---|---|---|---|---|---|---|---|---|
| BUN (mg/dL) | 22.8 | 20.6 | 21.2 | 19.9 | 20.8 | 21.9 | 20.2 | 21 | 19.6 | 5.00-28.00 |
| CREA (mg/dL) | 0.225 | 0.163 | 0.136 | 0.184 | 0.149 | 0.198 | 0.191 | 0.184 | 0.232 | 0.20-0.50 |
| BUN/CRE A Ratio | 101.333 | 126.380 | 155.882 | 108.152 | 139.597 | 110.606 | 105.759 | 114.130 | 84.483 | — |
| ALP (UL) | 70 | 90.6 | 47.2 | 35.4 | 67.8 | 49.8 | 93.9 | 85.6 | 65.5 | 105.00-370.00 |
| ALT (U/L) | 25.1 | 27.7 | 23.7 | 53.4 | 26.4 | 21.7 | 23.5 | 47 | 26.8 | 27.00-195.00 |
| AST (U/L) | 77.7 | 109.6 | 58.7 | 83.4 | 45.9 | 69.3 | 56.3 | 89 | 89.4 | 54.00-77.00 |
| GGT (U/L) | — | 0 | 0.3 | 0 | 0.1 | — | 0.5 | — | 0.4 | — |
| TBIL (mg/dL) | 0.156 | 0.148 | 0.202 | 0.157 | 0.237 | 0.155 | 0.126 | 0.133 | 0.109 | 0.20-0.60 |
| DBIL (mg/L) | 0.032 | 0.036 | 0.032 | 0.023 | 0.046 | 0.037 | 0.028 | 0.028 | 0.023 | — |
| IBIL (mg/dL) | 0.124 | 0.112 | 0.170 | 0.134 | 0.191 | 0.118 | 0.098 | 0.105 | 0.086 | — |
| TP (g/dL) | 4.93 | 4.62 | 4.1 | 4.65 | 4.49 | 4.52 | 4.26 | 4.28 | 4.29 | 4.80-7.20 |
| ALB (g/dL) | 3.07 | 2.9 | 2.39 | 2.63 | 2.62 | 2.67 | 2.71 | 2.72 | 2.63 | 2.40-4.30 |
| GLOB (g/dL) | 1.860 | 1.720 | 1.710 | 2.020 | 1.870 | 1.850 | 1.550 | 1.560 | 1.660 | 1.70-2.20 |
| A/G Ratio | 1.616 | 1.706 | 1.406 | 1.315 | 1.379 | 1.483 | 1.807 | 1.700 | 1.547 | — |
| P (mg/dL) | 7.86 | 7.43 | 7.45 | 6.26 | 7.19 | 7.28 | 8.05 | 6.5 | 8.42 | 7.30-14.50 |
| Ca (mg/dL) | 9.19 | 9.03 | 8.11 | 9.35 | 8.55 | 8.77 | 8.87 | 8.8 | 9.39 | 9.50-12.50 |
| GLU (mg/dL) | — | 178.4 | 106.3 | 149 | 164.6 | 194.3 | 123.4 | 139.9 | 159.4 | 172.00-372.00 |
| CHOL (mg/dL) | 55.9 | 46.3 | 65.5 | 96.9 | 70.7 | 64.2 | 42.9 | 47.7 | 38.9 | 55.00-169.00 |
| TRIG (mg/dL) | 131 | 125.2 | 165.7 | 125.9 | 151.6 | 168.1 | 86.1 | 48.5 | 85.3 | 67.00-289.00 |
| CK (U/L) | — | 611.9 | 161.4 | 111.1 | 77.5 | 223.1 | 82.4 | 190.9 | 171.6 | 428.00-1609.00 |
| TCO2 (mEg/L) | — | 11 | 17.5 | 18.2 | 8.2 | — | 14.9 | — | 12.1 | — |
| Na (mEq/L) | 160 | 150 | 154 | 148 | 153 | — | 151 | — | 153 | 145.00-181.00 |
| K (mEq/L) | 5.4 | 4.7 | 5.5 | 5.9 | 5.3 | — | 5.7 | — | 6 | 7.30-11.10 |
| CL (mEq/L) | 114 | 109 | 112 | 104 | 110 | — | 113 | — | 114 | 111.00-134.00 |
| Na/K | 30 | 32 | 28 | 25 | 29 | — | 26 | — | 26 | — |
| Anion Gap | — | 35 | 30 | 32 | 40 | — | 29 | — | 33 | — |

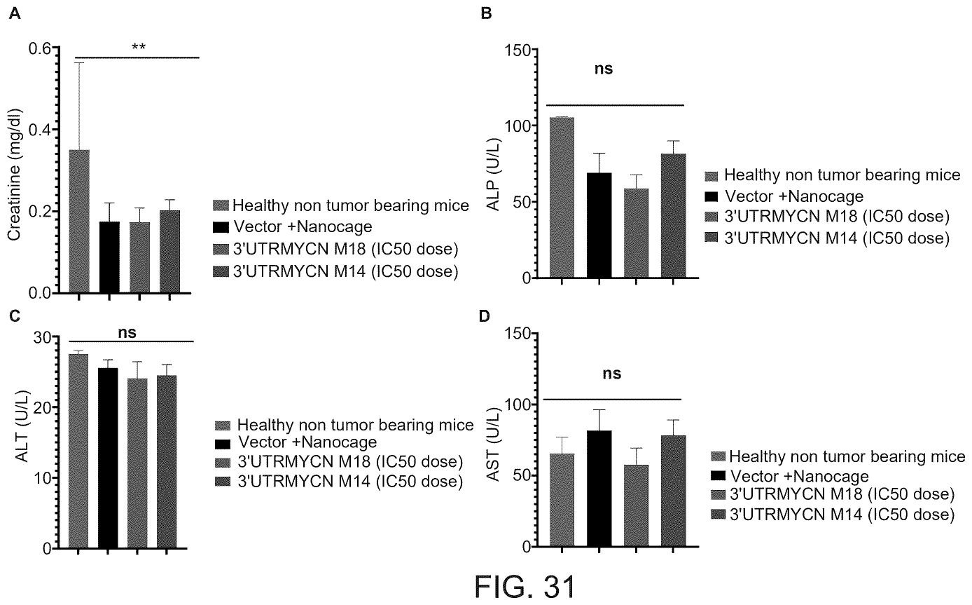

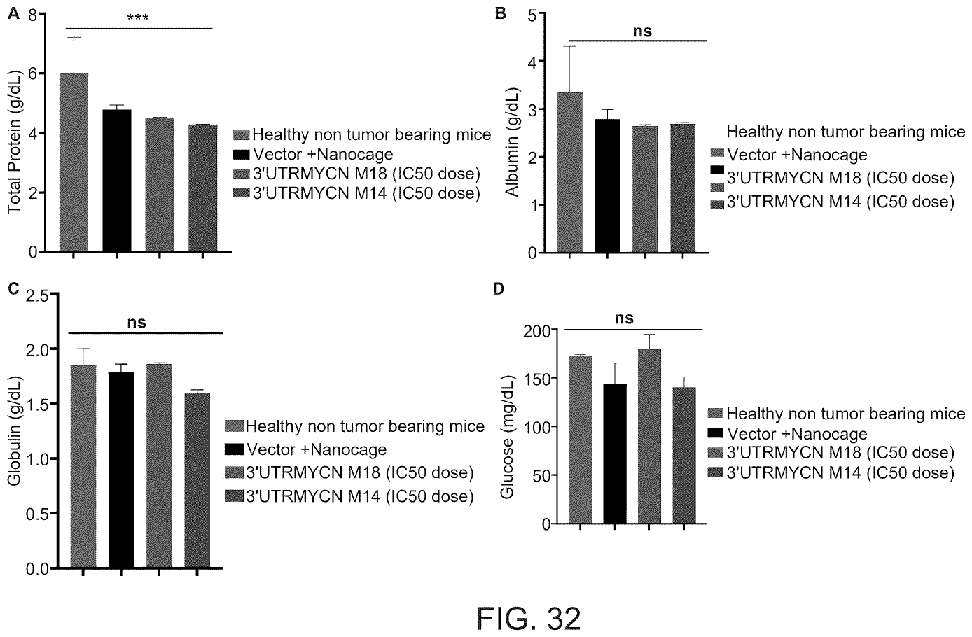

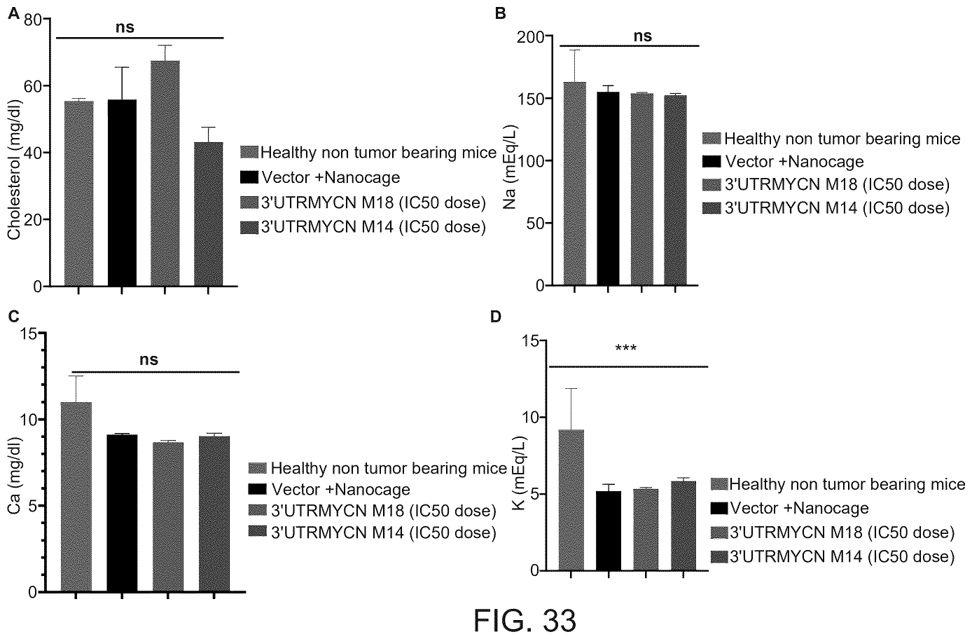

We then assayed for the kidney function. We found no change in blood urea nitrogen (FIG. 30D). The creatinine levels were reduced in the vector+nanocage treated groups as well as in the MYCN mRNA destabilizing drug treated groups (FIG. 31A) compared to the healthy untreated control. In the liver enzyme's function test, we found that the ALP were reduced in the vector+nanocage treated groups as well as in the MYCN mRNA destabilizing drug treated groups (FIG. 31B) compared the healthy untreated control. No changes were found in the levels of the ALT (FIG. 31C) and AST (FIG. 31D). We found no changes in the total protein level (FIG. 32A), albumin (FIG. 32B), glucose (FIG. 32C), bilirubin (FIG. 32D) or cholesterol (FIG. 33A) in the treated animals compared to the controls. In the electrolyte analysis, we found no changes in the sodium levels (FIG. 33B) while the calcium levels (FIG. 33C) and the potassium levels (FIG. 33D) were reduced in the vector+nanocage treated groups as well as in the MYCN mRNA destabilizing drug treated groups. Taken together, this demonstrates that MYCN mRNA destabilizing drugs are safe to administer, with no attendant abnormalities.

### Example 2C: Discussion

Neuroblastomas (NB) are tumors of the sympathetic nervous system and can arise from anywhere along the sympathetic chain ganglia (8a). They originate from the neural crest cells and can occur on the spine, chest, head, and adrenal glands. Globally, NB accounts for approximately 7-10% of all childhood cancers (8a). The worldwide incidence of neuroblastoma is estimated to be 10.2 cases per million children under 15 years of age 9. In high-income countries, neuroblastoma represents about 28% of all cancers diagnosed in infants. Neuroblastomas are broadly divided into high (HR), intermediate and low risk (LR) neuroblastomas (9a). The survival outcome for the low-risk neuroblastoma for 5 yrs is more than 75% whereas the outcome for the high-risk neuroblastoma is very low. About 25% of high-risk neuroblastoma expresses very high levels of MYCN, TERT and ALK11,12.

Rhabdomyosarcoma (RMS), a very aggressive malignant cancer and is the most common soft tissue sarcoma in children, accounting for approximately 3-4% of all childhood cancers globally (13a). The worldwide incidence of pediatric RMS is estimated to be 4-7 cases per million children per year. In high-income countries, the annual incidence is around 5.3 cases per million children. RMS arises from the mesenchymal tissues that failed to differentiate into normal healthy muscles. RMS are broadly divided into embryonal and alveolar rhabdomyosarcoma. RMS show high levels of MYCN amplification as well as MYOD1 and CDK4 and p53mut (14a, 15a, 16a). MYOD1 is a master gene involved in the development of muscle programs. In normal muscle development, the MYOD1 regulates differentiation from myotubes to muscle fibers in a tightly regulated program. In the RMS, the function of regulating differentiation is switched by mutations which leads MYOD1 to promote muscle cells proliferation and aggressive uncontrollable growth. The p53 is a tumor suppressor gene and the guardian of genome. Mutation in the p53 genes causes a loss of function of the tumor suppressor with a gain of oncogenic function. The mutant p53 is associated with aggressive rhabdomyosarcoma and drives the RMS malignant pleomorphism (26a).

The standard of care treatment of NB includes surgery, chemotherapy, radiotherapy, and targeted drugs. For RMS, the standard of care treatment includes surgery, radiation therapy and chemotherapy. MYCN has so far been indirectly targeted by the EZH2 inhibitors and BRD4 and HDAC inhibitors (17a, 18a, 19a). Since there is no clinically approved direct inhibitor of MYCN, there is a significant hurdle for addressing cancers having overexpression of MYCN. The dysregulation of the MYCN gene is correlated with rhabdomyosarcoma, osteosarcoma, neuroblastoma, and glioblastoma, as well as the development of breast cancer, small-cell lung cancer, prostate cancer, basal cell carcinoma, and acute lymphoblastic leukemia, among others.

We have presently investigated the MYCN 3′UTR and discovered that there are mRNA poly U elements present. We hypothesized that the destabilization of these elements on the 3′UTR of MYCN will lead to the destabilization of the MYCN transcript and hence triggering the downregulation of the MYCN protein.

We engineered the mRNA stabilization elements on the 3′UTR of MYCN into the unstable forms and thereby developed two novel MYCN destabilizing constructs designated 3′UTRMYCNM1-14 and 3′UTRMYCNM1-18. In vitro, these constructs achieved dose dependent down regulation of the MYCN for both the mRNA transcript and transcription factor binding levels with an IC₅₀ superior compared to the standard of care drugs such as epirubicin, olaparib, paclitaxel, cisplatin, actinomycin D and cyclophosphamide. This was demonstrated in MYCN over expressing neuroblastoma cell lines (Kelly and SKNBE2), in the rhabdomyosarcoma cell lines (RD) and in androgen independent prostate cancer cells (PC3). In vivo, 3′UTRMYCNM1-14 achieved inhibition of the aggressive metastatic rhabdomyosarcoma primary tumor and lung metastasis with very significant survival outcomes. The MYCN mRNA destabilizing drugs exhibited safety in vivo, were well distributed, and caused no abnormal blood dyscrasia nor changes in the electrolyte, liver, kidney, pancreatic or gallbladder functions. Taken together, these results point to a first-in-class MYCN mRNA destabilizing drug with in vivo therapeutic efficacy and acceptable safety profile for use in the inhibition of the metastatic aggressive rhabdomyosarcoma.

To the best of our knowledge, this is the first direct targeting of the MYCN mRNA on the MYCN 3′UTR. Competing drugs to target MYCN exist include MYCN-A3 which is a DNA alkylating agent that reduced the copy number of the amplified MYCN in neuroblastoma cells (24a). Another drug indirectly targets MYCN through PAX3/FOXO1 interaction with the antigene peptide nucleic acid (PNA) in rhabdomyosarcomas (25a). The EZH2 inhibitor has also been used target MYCN amplified neuroblastomas (17a). Given that these drugs have been shown to only target MYCN in neuroblastoma or rhabdomyosarcoma, the 3′UTRMYCN mRNA destabilizing drug might represent the pan-MYCN targeting drug. Consistent with this, we have shown therapeutic efficacy in vitro and in vivo in the metastatic MYCN rhabdomyosarcoma and in vitro in the MYCN amplified neuroblastoma cells and in the androgen independent prostate cancer cells.

Notably, we have achieved a survival outcome of 84 days in vivo in mice. To put this into perspective, one day of a mice's life is equivalent to 40 days of a human's life. Therefore, 84 days of survival in mice is equivalent to 3,360 days of survival in humans. This is 9 yrs survival outcome as a monotherapy for MYCN driven rhabdomyosarcoma, and clearly superior to the standard of care for rhabdomyosarcoma which offers 70-90% 5 yr survival outcome in the non-aggressive RMS tumors.

In conclusion, we achieved complete pathological response in the MYCN driven rhabdomyosarcoma. We used NSG xenograft model to perform the in vivo work. Our next steps will be to extend the assessment of the drug in MYCN driven rhabdomyosarcoma GEMM models and also in additional RMS MYCN cell lines. From the current studies, the 3′UTRMYCNM1-14 drug showed efficacy in vitro and in vivo. We will assess in vivo efficacy of the 3′UTRMYCNM1-18 drug in neuroblastoma and in androgen independent prostate cancers, as well as other cancer cells.

### Example 3: The 3′UTRMYC1-18 mRNA Destabilizing Drug Achieves Titratable In Vivo Dose Dependent Inhibition of c-MYC Positive Triple Negative Breast Cancer (TNBC) and Other Cancers with Significant Survival Outcomes and Safety Profiles

Overview: c-MYC is a master transcription factor that belongs to the basic helix loop helix family. It has been implicated as over expressed in more than 70% of human cancers. Yet, there is no clinically approved direct inhibitor of c-MYC. We report the development of a novel 3′UTRMYC1-18 drug for achieving targeted dose dependent titratable inhibition of c-MYC in ethnically diverse triple negative breast cancer. In vivo, we treated TNBC tumor bearing mice with: 1) vector+nanocage control 2) 3′UTRMYC1-18 at the IC₅₀ dose (8.75 μg) 3) 3′UTRMYC1-18 at the 2×IC₅₀ dose (17.5 μg) 4) 3′UTRMYC1-18 at the 4×IC₅₀ dose (35.0 μg) 5) 3′UTRMYC1-18 IC₅₀ dose (8.75 μg)+olaparib IC₅₀ (9.5 μg). We show that the MYC mRNA destabilizing drug inhibited the tumors in a dose dependent manner, with the 35 μg dosage being most effective. Both short-and -long term analyses of toxicity profiles were obtained by assaying blood cell count, chemistry, electrolyte, liver, kidney, and gall bladder and pancreatic function. We demonstrate that the drug is safe and well tolerated.

### Example 3A: Materials and Methods

Construction methods for 3′UTRMYC1-18 are described in Awah et al. 2024, PMC11311709.

DHCA-coated IO-nanocage synthesis: The IO-nanocages were synthesized with oleic acid by a modified version of a previously published method (Nano Lett. 2016, 16, 7357-7363, (Nano Lett., 2016, 16, 7357-7363; I. Am. Chem. Soc., 2014, 136, 12552-5; Oncol. Rep., 2020, 43, 169-176; Nat. Commun., 2021, 12, 2903). Manganese (II) acetate (0.17 g), oleylamine (0.82 mL), and oleic acid (0.16 mL) were added to p-xylene (15 mL) in a three-necked 50 mL flask with a reflux condenser and sonicated for 10 mins. The flask was heated to 90° C. in air under magnetic stirring, then 1 mL of deionized water was rapidly injected into the flask. The reaction mixture was heated at 90° C. for 1.5 hrs, producing MmCU nanoparticles. 1 mL of 2.4 M aqueous iron (II) perchlorate solution was added and the mixture maintained at 90° C. for an additional 1.5 hours to produce IO-nanocages by galvanic replacement. After cooling, IO-nanocages were collected by centrifugation, rinsed with ethanol, and dispersed in THF.

The hydrophobic IO-nanocages were coated with DHCA and transferred to the aqueous phase using a modified version of a previously published method. (Liu et al., J. Am. Chem. Soc. 2014, 136, 12552-5). First, 100 mg of DHCA was dissolved in 5 mL of THF in a three-neck flask (25 mL). The resulting solution was heated to 50° C. after bubbling for 30 seconds with flowing nitrogen gas. Then, 20 mg of hydrophobic IO-nanocages capped by oleic acid were dispersed in 1 mL of THF which was added to the solution. The solution was heated to 50° C. for 3 hours, then cooled to room temperature, and 500 pL NaOH (0.5 M) was introduced to precipitate the magnetic nanoparticles. The precipitate was collected by centrifugation and redispersed in 2 mL water, then dialyzed overnight.

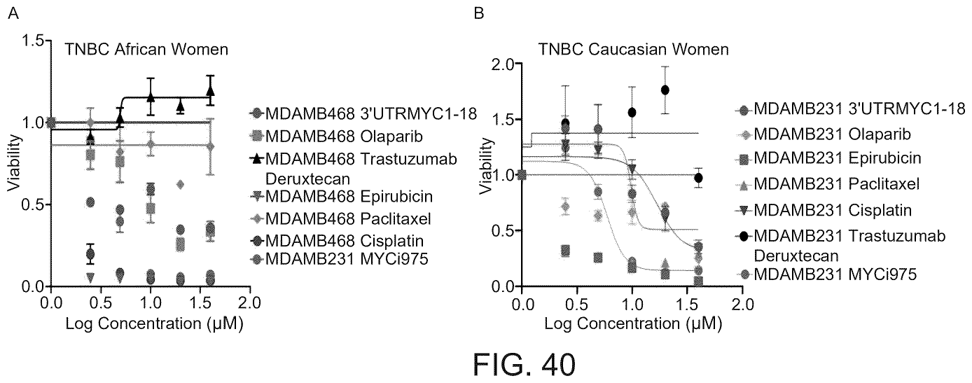

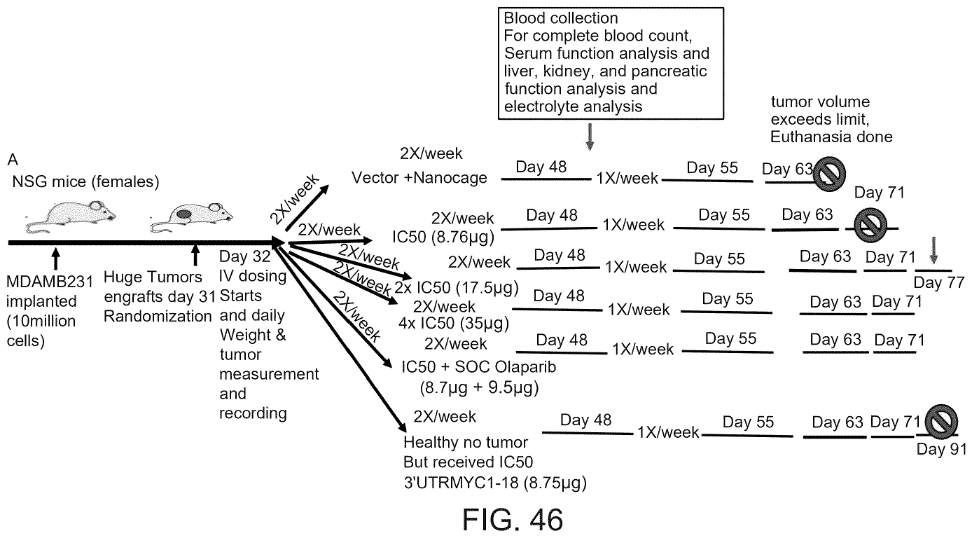

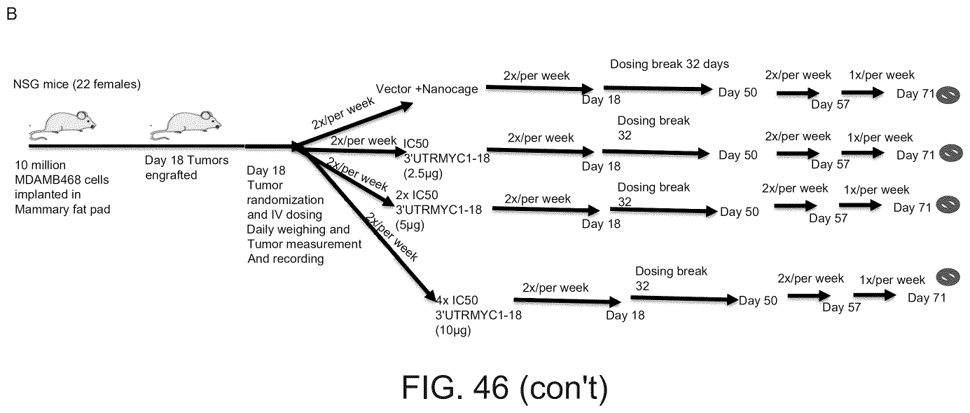

Experimental design: To prove the dose dependent inhibition of the tumors by 3′UTRMYC1-18, we set up an in vivo dose titration experiment in TNBC. We obtained IACUC approval for the animal work from the institutional board of the City University of New York. We obtained 25 female NSG (NOD SCID mice) from the Jackson Laboratory. We allowed the animals to acclimate according to the institutional protocol. We cultured the TNBC cells to 80% confluency in DMEM media. We harvested the cells and washed the cells in 1× phosphate buffer saline (PBS) and then mixed them in a 1:1 ratio with Matrigel (Corning). We then implanted 10 million cells into the mammary fat pad of the mice. After 31 days the tumors engrafted (FIG. 46A). We randomized the animals according to tumor size and weight to ensure equal starting tumor volumes into 6 groups: 1) 5 mice—vector+nanocage, 2) 5 mice—IC₅₀ dose (8.75 μg of 3′UTRMYC1-18 for TNBC (FIG. 40B, Table 3A), 3) 5 mice—2×IC₅₀ dose (17.5 μg of 3′UTRMYC1-18), 4) 4 mice 4×IC₅₀ dose (35 μg of 3′UTRMYC1-18), 5) 4 mice—2×IC₅₀ dose (8.75 μg of 3′UTRMYC1-18)+IC₅₀ olaparib (9.5 μg, FIG. 40B, Table 3A), 6) 2 mice—healthy with no tumor treated with the IC₅₀ 3′UTRMYC1-18 (8.75 μg).

TABLE 3A
IC₅₀ dose determinations

| Cancer cells | 3′UTR- | | | | | |
|---|---|---|---|---|---|---|
| IC₅₀ (μM) | MYC1-18 | olaparib | MYCi975 | epirubicin | paclitaxel | cisplatin |
| Mia-Paca2 pancreas | 1.98 | >40 | 10 | 2 | >40 | 8 |
| MDAMB468 breast | 2.5 | 8 | 6 | Unstable | >40 | Unstable |
| PC3 pancreas | 2.5 | 2.5 | 10 | 25.7 | 20 | 15.49 |
| A2780 ovarian | 4.47 | >40 | 20 | Unstable | Unstable | 7.8 |
| E006AAht prostate | 0.77 | 14 | 4 | 15 | 6 | 15 |
| MDAMB231 breast | 8.76 | 9.5 | 7 | 0.2 | 0.2 | 9.5 |
| 22RV1 prostate | 2.5 | 2.5 | 10 | 9 | >40 | 22 |
| NCIH1975 lung | 9.8 | 20 | 5.5 | Unstable | >40 | 7.6 |
| PANC1 pancreatic | >20 | 8 | 5 | 1.8 | 1.8 | 8 |
| PSN1 pancreatic | 1.2 | 1.2 | 5.0 | 1.2 | 1.2 | 1.2 |
| SUM1315 breast | 20 | 20 | | 0.4 | 20 | 0.4 |
| COLO 320 DM colorectal | 1.88 | >40 | | 22 | >40 | 9 |
| MESSA uterine | 8.32 | 2 | | Unstable | 1.98 | Unstable |
| RWPE1 prostate | 19.07 | 10 | | 5 | 10 | 2.5 |
| AC16 cardiomyocyte | 10 | 10 | 1.9 | 2.5 | >20 | |
| NCIH524 lung | 2.8 | 2.8 | | 2.8 | 2.8 | 2.8 |
| HEC1A uterine | 2 | >20 | | >20 | >20 | >20 |
| SKNPDW neuronal | 5 | 2.5 | | 1.5 | >20 | 2.5 |

| Cancer cells | | | | | trastuzumab |
|---|---|---|---|---|---|
| IC₅₀ (μM) | abiterone | enzalutamide | bevacizumab | osimertinib | deruxtecan |
| Mia-Paca2 pancreas | | | | | |
| MDAMB468 breast | | | | | 6 |
| PC3 pancreas | 40 | 30.53 | | | |
| A2780 ovarian | | | 18.22 | | |
| E006AAht prostate | 15 | 15 | | | >40 |
| MDAMB231 breast | | | | | |
| 22RV1 prostate | 20 | >20 | | | |
| NCIH1975 lung | | | | 0.4 | >40 |
| PANC1 pancreatic | | | | | |
| PSN1 pancreatic | | | >40 | | |
| SUM1315 breast | | | >40 | | >40 |
| COLO 320 DM colorectal | | | >40 | | |
| MESSA uterine | | | >40 | | |
| RWPE1 prostate | 10 | 5 | >10 | 5 | 10 |
| AC16 cardiomyocyte | >20 | >20 | >20 | 2.5 | >20 |
| NCIH524 lung | | | | | |
| HEC1A uterine | | | | | |
| SKNPDW neuronal | | | | | |

The dosing regimen was carried out 2× per week intravenously (tail vein) from days 32-48 (16 days). This was followed by 1λ/week dosing regimen between days 48-55 (1 week). Thereafter, we stopped dosing, only the 4×IC₅₀ 3′UTRMYC1-18 (35 μg) received 1× per 2 weeks between days 63-71 (8 days) because this group missed the dose of the days 48-55. On day 63, the vector plus nanocage group exceeded the tumor limit and were euthanized. On day 71, the IC₅₀, the 4×IC₅₀ 3′UTRMYC1-18 and the combo group (IC₅₀ 3′UTRMYC1-18+IC₅₀ Olaparib) were euthanized. The 2×IC₅₀ group survived until day 77. The healthy group with no tumors but treated with IC₅₀ 3′UTRMYC1-18 were euthanized on day 91 (FIG. 46A). To assess the effect of the c-MYC mRNA destabilizing drug on tumor bearing and healthy animals, we collected blood at two time points. This included 2 weeks of dosing (day 48) and a late time point of 7 weeks of dosing (day 77) (FIG. 46A). We analyzed complete blood count, electrolyte, liver, kidney, and pancreatic function. The results are presented below.

Daily tumor volume measurement and recording: To obtain the daily tumor volumes, we used callipers and measured the tumor length and width. Using the formula (½×L×W×W), we calculated the tumor volume. We recorded the survival outcome from the controls and the different dose treated groups and plotted the outcome in a Kaplan-Meier curve. This methodology is described in detail in Awah et al. 2024, PMC11311709.

To validate the dose dependent inhibition in vivo, we performed H&E staining of the tumors from the vector+nanocage treated group, the IC₅₀ 3′UTRMYC1-18 (8.76 μg) treated group, the 2×IC₅₀ 3′UTRMYC1-18 (8.76 μg) treated group, and the 4×IC₅₀ 3′UTRMYC1-18 (35.0 μg) treated group. Briefly, tumors were prepared and embedded according to standard protocol. Tissues were sectioned in microtome and H&E staining was performed according to standard histology protocol. Images of the stained tissues were obtained using the EVOS FL microscope at 40× magnification and quantification was done in Image J (RRID:SCR_003070)(https://imagej.net/ij/). Data were quantified in Graph Pad Prism (v10) RRID:SCR_002798.

We sought to validate in vivo that 3′UTRMYC1-18 destabilized and inhibited the oncogenic c-MYC in a dose dependent titratable manner. For this, we performed c-MYC IHC staining of the tumors from the positive c-MYC control tumor tissue, vector+nanocage treated group, the IC₅₀ 3′UTRMYC1-18 (8.76 μg) treated group, the 2×IC₅₀ 3′UTRMYC1-18 (8.76 μg) treated group, and the 4×IC₅₀ 3′UTRMYC1-18 (35.0 μg) treated group. We also performed an RIC staining of the PD-L1 in the vector+nanocage treated mice and in the IC₅₀ 3′UTRMYC1-18 (8.76 μg) treated group, the 2×IC₅₀ 3′UTRMYC1-18 (8.76 μg) treated group, and the 4×IC₅₀ 3′UTRMYC1-18 (35.0 μg) treated group. To perform TIC staining against MYC and PD-L1 on the tumors and organs of the treated animals and the controls, we used the Abcam IHC protocol (https://www.abcam.com/en-us/technical-resources/protocols/ihc-with-samples-in-paraffin). We deparaffinized the tissue slides according to the protocol and performed enzymatic antigen retrieval using 1:1 trypsin concentrates and buffer. Washes were done with 1×TBST. Blocking was done with protein block (ab64212) for 1 hr. After this, washes were performed again. Next, we incubated the slides with primary antibodies against MYC (Anti-c-MYC 1:1000, cat no: 67447-1-lg) or with PD-L1 (E1L3N®) XP® rabbit mAb #13684) overnight. Next day, slides were washed with 1× TBST. The secondary antibody was added on the slide and incubated for 1 hr. Subsequently, we washed the slides with 1×TBST. To detect the signals, we used the DAB and concentrated and enhanced with enhancer. After this, we counter stained and mounting media was added. Then, the cover slip was added and sealed. Images were obtained on EVOS Fl at 40×. Target staining of the MYC and PD-L1 was quantified in Image J (https://imagej.net/ij/) RRID:SCR 003070.

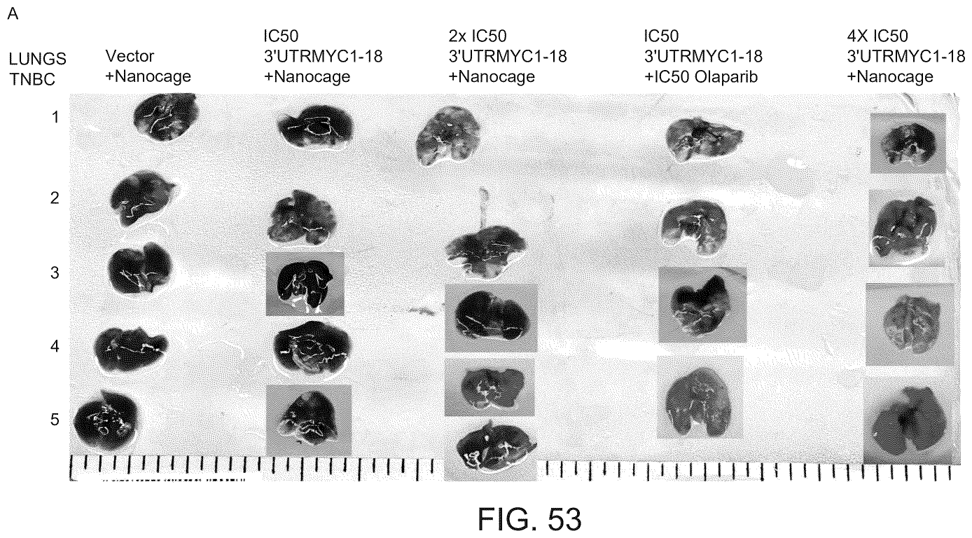

We performed full necropsy of the lungs of the tumor bearing mice treated with 3′UTRMYC1-18 in a dose dependent manner as well as the controls and combination with olaparib. To validate lung metastatic tumor inhibition in vivo by 3′UTRMYC1-18 in dose dependent treatment (FIG. 53), we performed H&E staining of the lungs from the vector+nanocage treated group, the IC₅₀ 3′UTRMYC1-18 (8.76 μg) treated group, the 2×IC₅₀ 3′UTRMYC1-18 (8.76 μg) treated group, and the 4×IC₅₀ 3′UTRMYC1-18 (35.0 μg) treated group. We also performed c-MYC IHC staining of the lungs from the vector+nanocage treated group, the IC₅₀ 3′UTRMYC1-18 (8.76 μg) treated group, the 2×IC₅₀ 3′UTRMYC1-18 (8.76 μg) treated group, and the 4×IC₅₀ 3′UTRMYC1-18 (35.0 μg) treated group. See staining methodology noted above.

We examined the livers and brains of the treated animals and the controls to see if there was a dose dependent in inhibition of metastasis by 3′UTRMYC1-18. We stained the brains of the treated animals and the controls with H&E. We also performed a c-MYC IHC staining of the livers from the vector+nanocage treated mice and in the IC₅₀ 3′UTRMYC1-18 (8.76 μg) treated group, the 2×IC₅₀ 3′UTRMYC1-18 (8.76 μg) treated group, and the 4×IC₅₀ 3′UTRMYC1-18 (35.0 μg) treated group. In addition, we extended analysis into whether we inhibited brain metastasis. We performed IC staining for the c-MYC for the brains of the treated groups and the controls. See staining methodology noted above.

To understand the pharmacokinetics of the drug in vivo in the tumor bearing mice, we administered the mRNA destabilizing drug the IC₅₀ 3′UTRMYC1-18+IO nanocage (8.7 μg) intravenously and collected the blood for time point 0, 3 hrs, 6 hrs and then 1 week. We collected the serum and then performed detection of the iron oxide using ELISA based assay. Using a plate reader and standard methodology, we read the absorbance intensity for the various time points.

We next tested for cachexia from 3′UTRMYC1-18 treatment by weighing and recording animal weights. To understand the toxicity and safety profile of 3′UTRMYC1-18 both after the short- and long-term dosing which were 16 days and 45 days respectively, we assayed the red blood cell count, hemoglobin concentration, hematocrit levels, mean corpuscular volume, mean corpuscular hemoglobin, mean corpuscular hemoglobin concentration, reticulocyte levels, platelet count, blood urea nitrogen levels, creatine levels, aspartate aminotransferase levels, alkaline phosphatase levels, alanine aminotransferase levels, total bilirubin, total protein levels, albumin levels, globulin levels, glucose levels, cholesterol levels, sodium levels, potassium levels, and calcium levels.

Experimental design for in vivo intravenous (tail vein) dose dependent dosing of c-MYC TNBC (MDAMB468-African descent woman): To prove the general efficacy of the c-MYC mRNA destabilizing drug for c-MYC driven TNBC, we set up an in vivo dose dependent titration of 3′UTRMYC1-18 in TNBC from a woman of African descent (MDAMB468). We obtained IACUC approval for the animal work from the institutional board of the City University of New York. We obtained 22 female NSG (NOD SCID mice) from the Jackson Laboratory. We allowed the animals to acclimate according to the institutional protocol. We cultured the TNBC cells to 80% confluency in DMEM media. We harvested the cells and washed in 1× phosphate buffer saline (PBS). We then mixed the cells in 1:1 ratio with the Matrigel (Corning). We implanted 10 million cells into the mammary fat pad of the mice. After 18 days, the tumors were engrafted (FIG. 46B). We randomized the animals according to tumor size and weight to ensure equal starting tumor volumes into 4 groups namely 1) 6 mice—vector+nanocage, 2) 4 mice—IC₅₀ dose (2.5 μg of 3′UTRMYC1-18), 3) 8 mice—2×IC₅₀ dose (5 μg of 3′UTRMYC1-18), 4) 4 mice—4×IC₅₀ dose (10 μg of 3′UTRMYC1-18; FIG. 46B).

### Example 3B: Results

3′UTRMYC1-18 IC₅₀ determination in ethnically diverse TNBC. c-MYC driven cancers are extremely aggressive and highly metastatic. To establish the therapeutic efficacy and the dosage of 3′UTRMYC1-18 across diverse pan human cancers regardless of tissue of origin and genetic/ethnic background of the cancer. We performed a head-to-head dose dependent titration for IC₅₀ determination of 3′UTRMYC1-18 in comparison with standard of care drugs: PARPi (olaparib), TOP2i (epirubicin), taxane (paclitaxel), alkylating agent (cisplatin), HER2/neu/topoisomerase ADC (trastuzumab deruxtecan), EGFRi (osimertinib), VEGFi (bevacizumab), anti-androgen hormone therapy and ARi (abiraterone and enzalutamide) and MYC-Max protein inhibitor (MYCi975) (FIG. 40) in TNBC (MDAMB468-African descent woman, MDAMB231-Caucasian descent woman).

In the TNBC (MDAMB468), 3′UTRMYC1-19 achieved an IC₅₀ of 2.5 μM ahead of ahead of olaparib, epirubicin, paclitaxel, cisplatin, and trastuzumab deruxtecan (FIG. 40A). 3′UTRMYC1-19 showed superior IC₅₀ as compared to MYCi975 (a MYC-Max inhibitor) (FIG. 40A, Table 3A, above). In fact, the trastuzumab deruxtecan appeared to cause the proliferation of the TNBC confirming that it is negative for HER2 (FIG. 40A). In the TNBC (MDAMB231), 3′UTRMYC1-18 achieved an IC₅₀ of 8.76 μM superior to olaparib, cisplatin and trastuzumab deruxtecan (FIG. 40B, Table 3A, above). As before, trastuzumab deruxtecan appeared to cause proliferation of the TNBC.

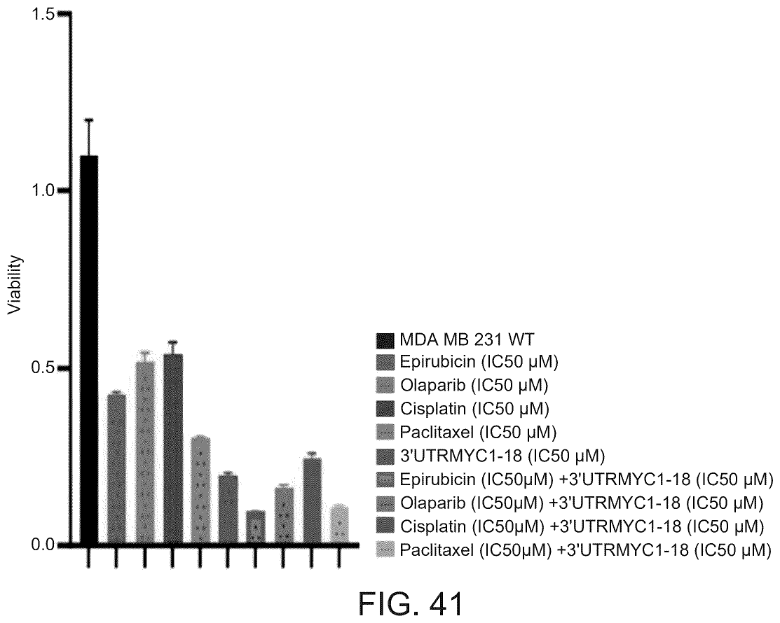

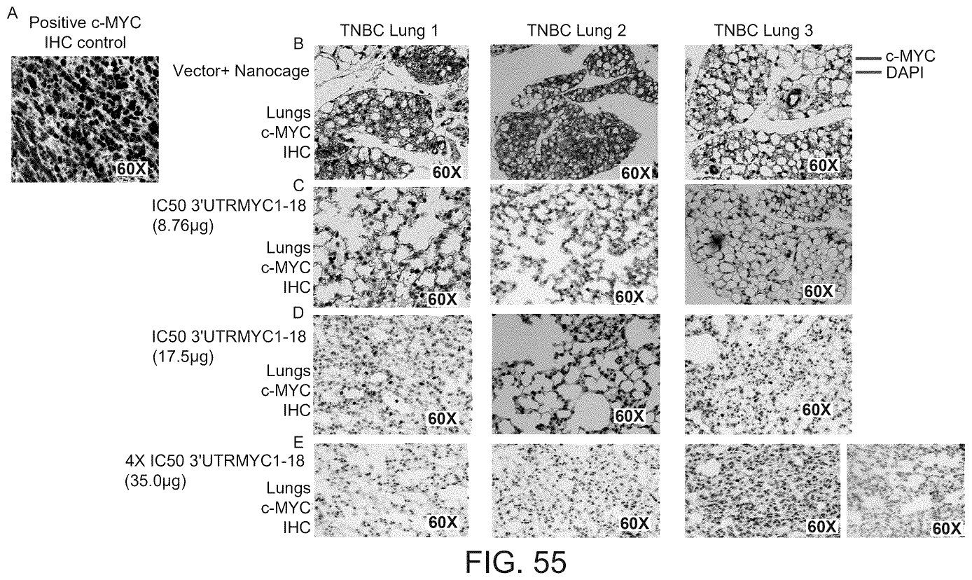

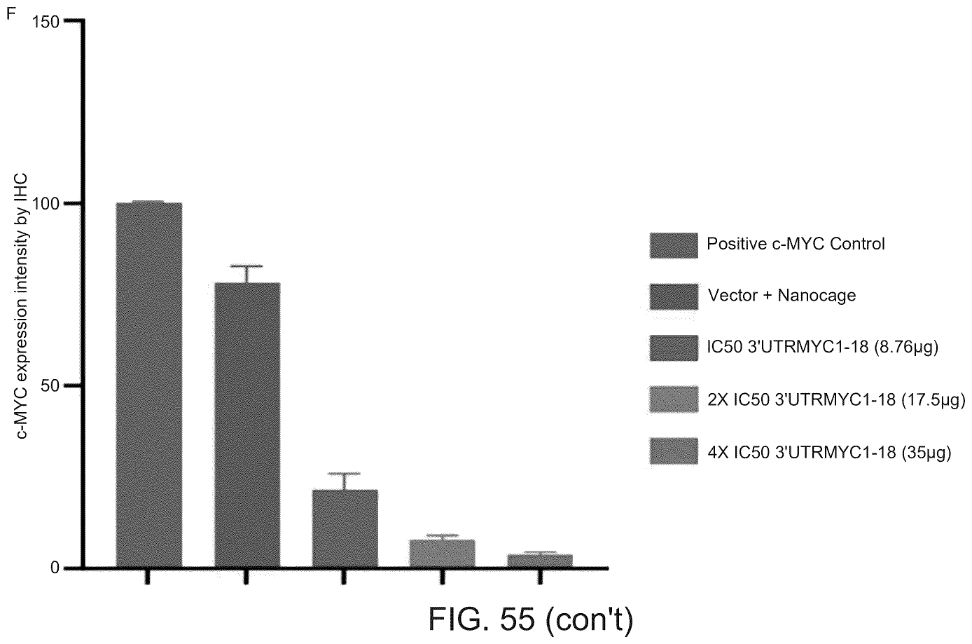

3′UTRMYC1-18 synergizes in a rational combination therapy with some standard of care chemotherapy. Because cancer patients receive combination therapy, we sought to determine the doses at which 3′UTRMYC1-18 synergizes with the standard of care drugs. We assessed the administration of 3′UTRMYC1-18 in combination with epirubicin, olaparib, cisplatin, paclitaxel in the triple negative breast cancer cells (MDAMB231; FIG. 41A-C). We found that 3′UTRMYC1-18 treatment synergized with epirubicin treatment, as well as with olaparib and paclitaxel treatment of TNBC (FIG. 55C). In summary, the results demonstrate that this mRNA destabilizing drug can be combined with the standard of care drugs in very aggressive cancers.

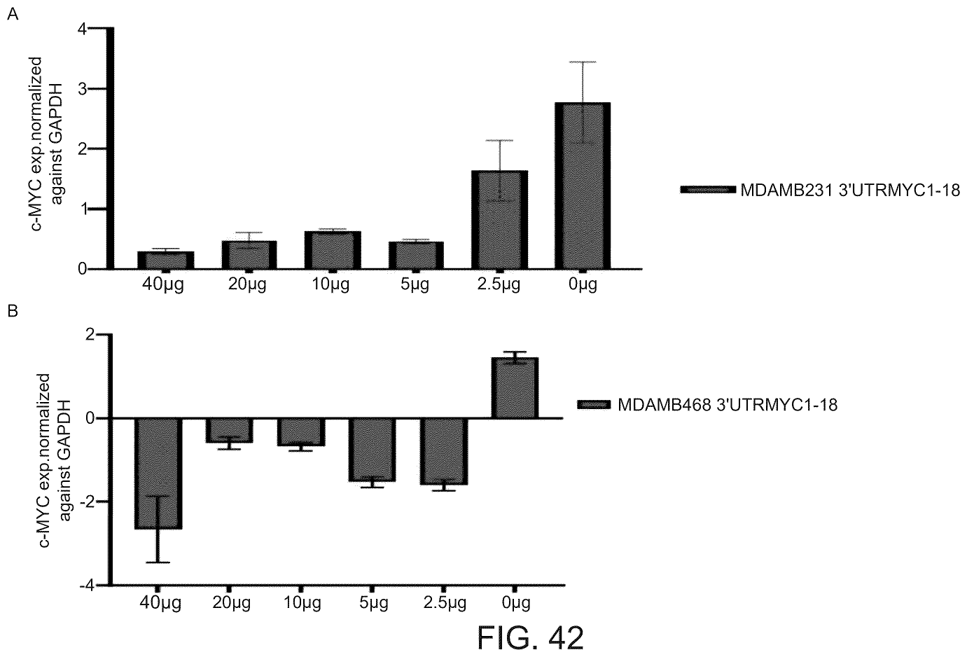

3′UTRMYC1-18 engages and degrades the MYC mRNA in a titratable dose dependent manner. To demonstrate that 3′UTRMYC1-18 engages the endogenous MYC mRNA in a dose dependent manner, we treated the ethnically diverse triple negative breast cancer (MDAMB231, MDAMB468), with dose dependent 3′UTRMYC1-18. We found that the construct downregulated the c-MYC mRNA in a titratable dose dependent manner compared to the controls (FIG. 42A-B).

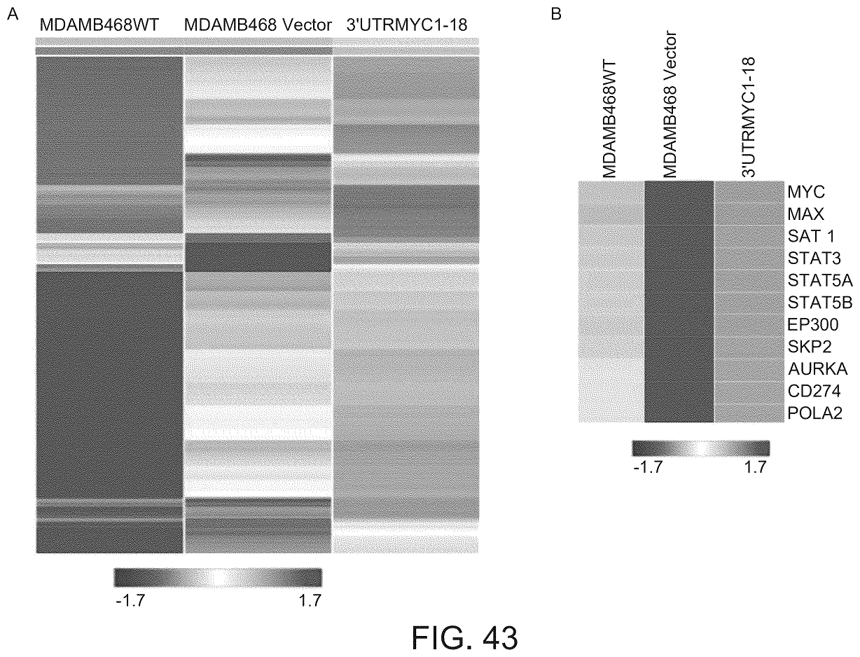

RNA seq validates 3′UTRMYC1-18 the specific down regulation of the c-MYC and it's interactome. To validate the down regulation of the c-MYC on the genome wide scale, we performed RNA sequencing on: 1) MDAMB468 WT cells, 2) vector treated cells and 3) 3′UTRMYC1-18 treated cells. We found that the MYC mRNA destabilizing drug downregulated c-MYC and its interactome (FIG. 43A-B).

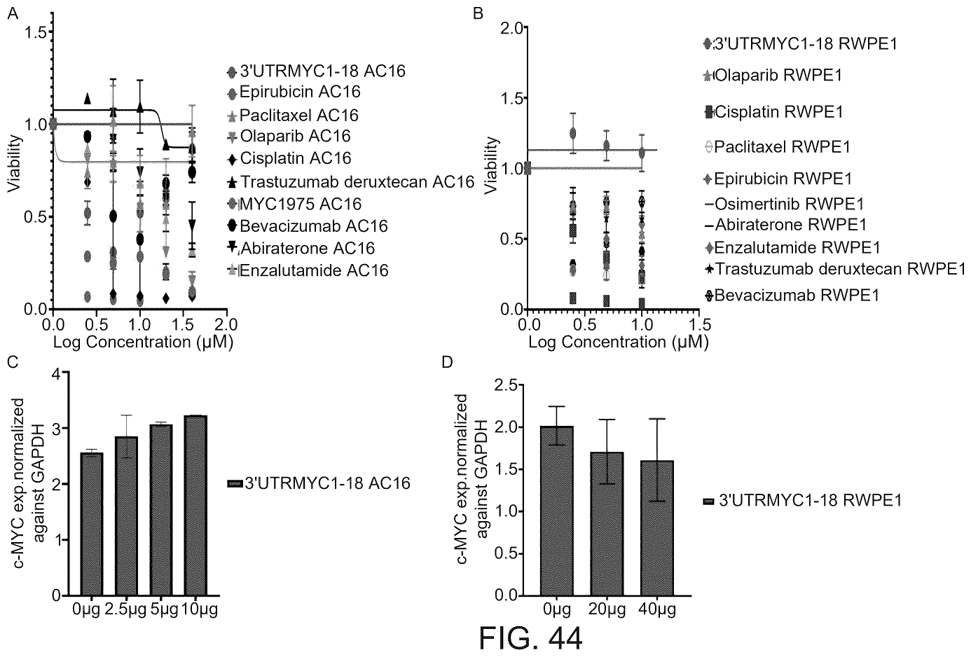

3′UTRMYC1-18 is safe for the normal healthy cardiomyocytes and epithelial cells. We next sought to prove the safety of the engineered 3′UTRMYC1-18 to normal healthy cells of the body. We performed a dose dependent titration of the drug 3′UTRMYC1-18 in a head-to-head comparison with the standard of care drugs and MYCi975 in the normal cardiomyocytes (AC16 cells) and the normal epithelial cells (RWPE1) (FIG. 44A-B). We found that the 3′UTRMYC1-18 drug was not toxic to normal cardiomyocytes and epithelial cells. By comparison, the standard of care drugs and MYCi975 were toxic to the healthy cells (FIG. 44A-B). On the mRNA level, we found that the 3′UTRMYC1-18 drug did not down regulate the MYC expression in these healthy cells (AC16 and RWPE1) (FIG. 44C-D).

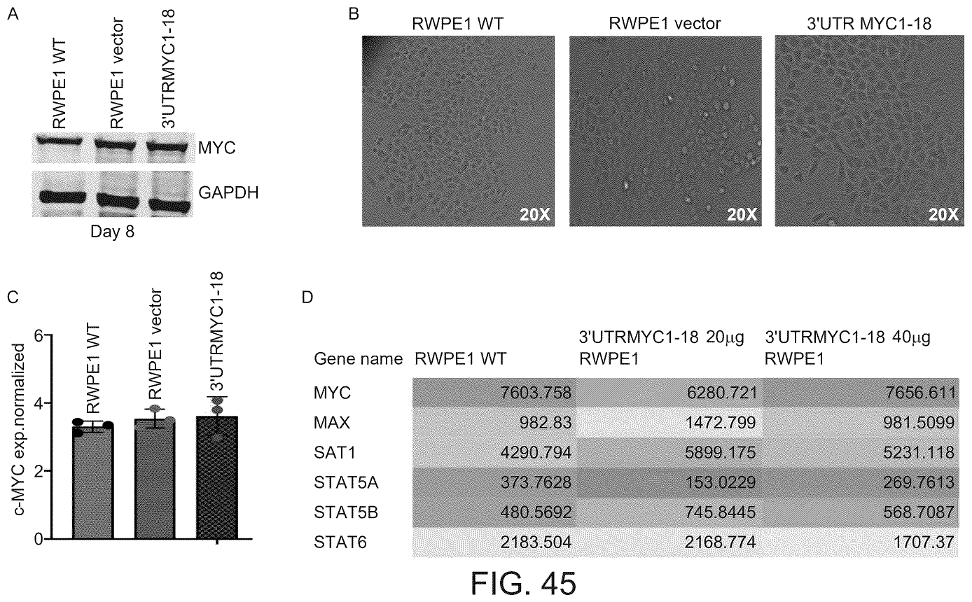

Having demonstrated that 3′UTRMYC1-18 is safe for administration, we extended the analysis to western blot, cellular morphological changes, and mRNA expression by qPCR and RNA seq analysis. We found that the construct does not degrade c-MYC protein, mRNA or change the morphology of the normal healthy cells (FIG. 45A-D). In addition, we found in global RNA analysis that the MYC, MAX and its other direct interactors do not change in normal healthy cells treated with the 3′UTRMYC construct. This data demonstrates the safety of 3′UTRMYC1-18 therapy in normal healthy cells.

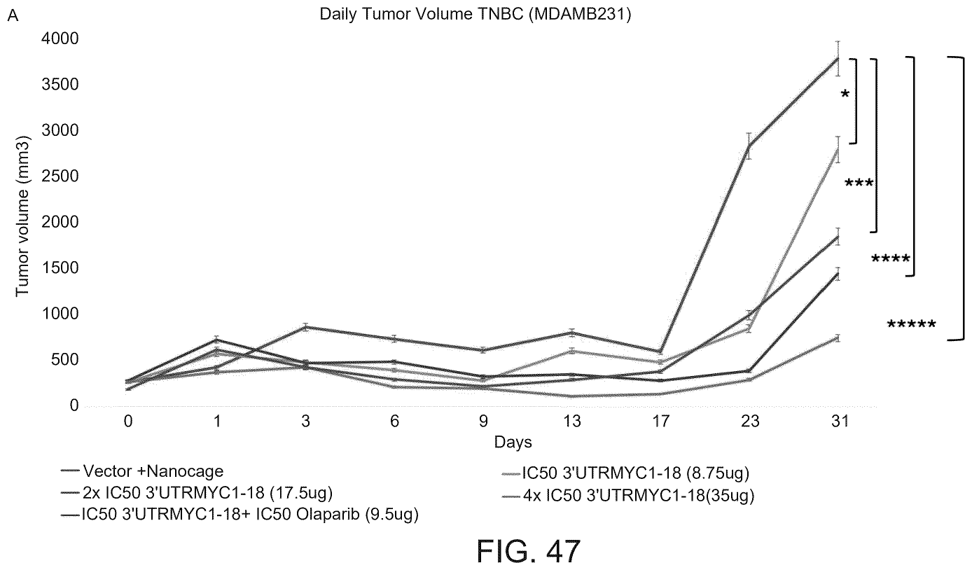

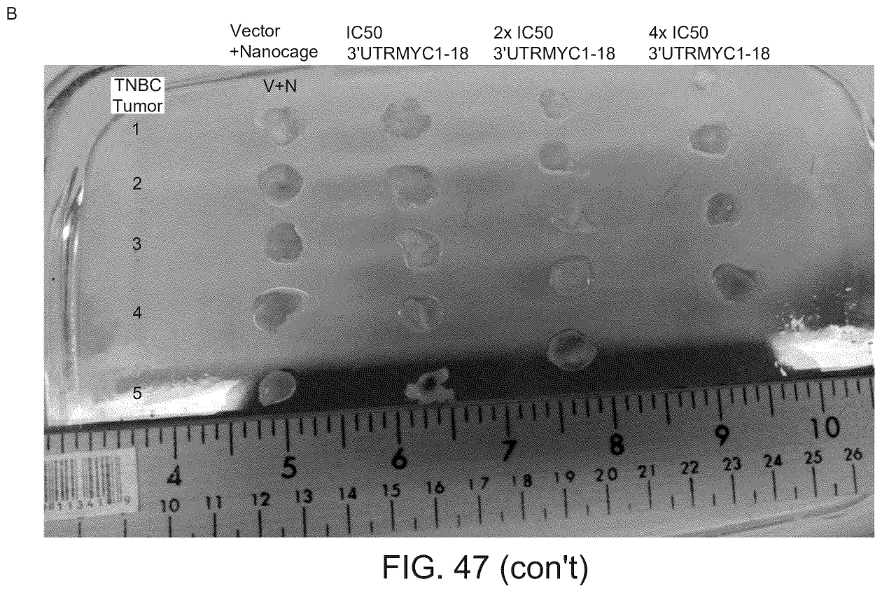

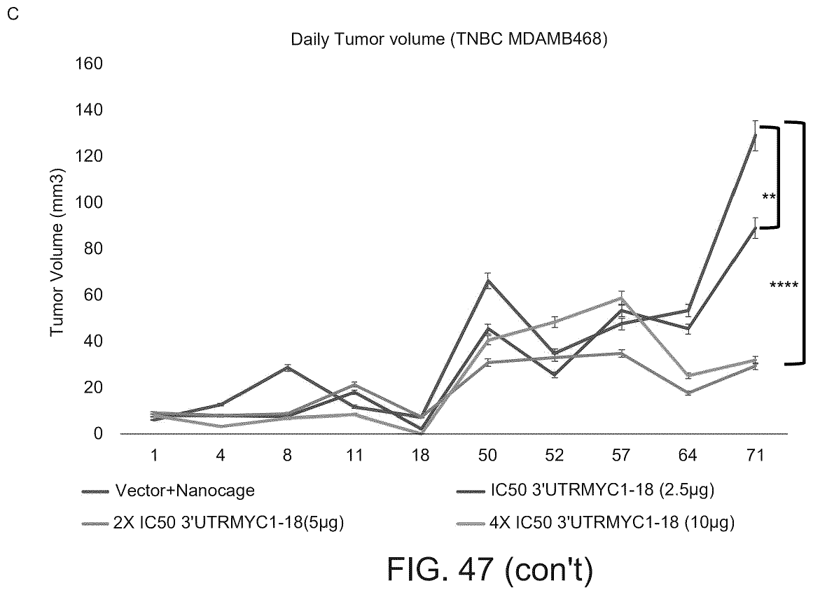

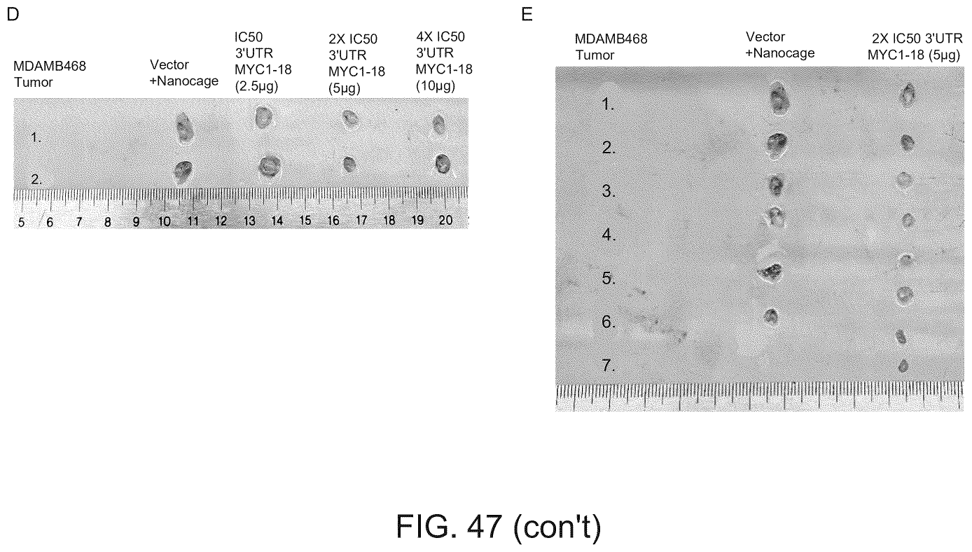

3′UTRMYC1-18 inhibits pan c-MYC driven tumors in vivo in dose dependent titratable manner as assessed in a intravenous clinical route of administration (tail vein IV). Using the experimental design described above, we found that 3′UTRMYC1-18 inhibited tumors in a dose dependent manner 4×IC₅₀>2×IC₅₀>IC₅₀ (FIG. 47A). Further, we found that 3′UTRMYC1-18 inhibited the TNBC cells in a titratable dose dependent manner (FIG. 47B).

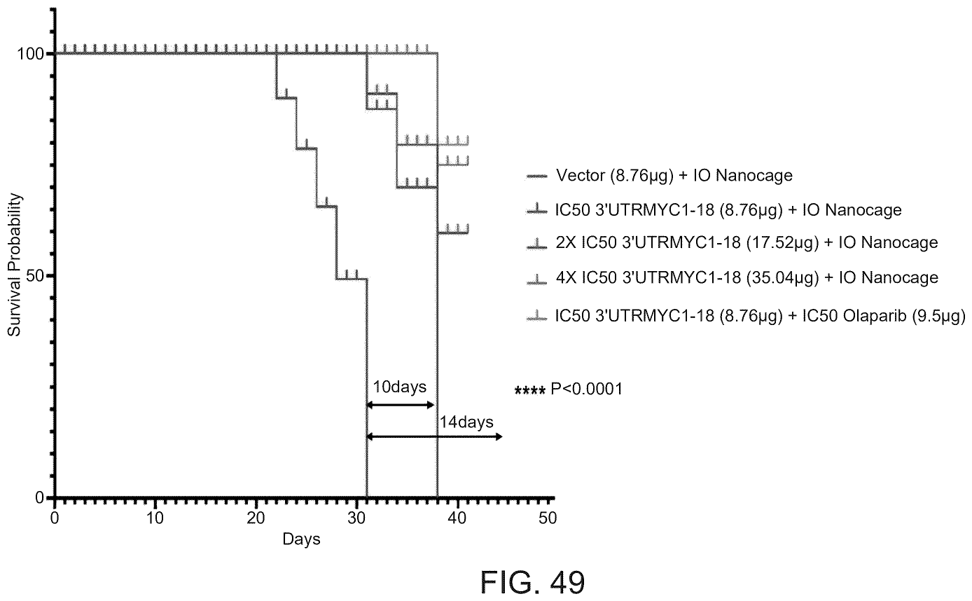

3′UTRMYC1-18 significantly improves the survival outcome of the TNBC in a titratable dose dependent manner. We recorded the survival outcome from the controls and the different dose treated groups and plotted the outcome in a Kaplan-Meier curve. We found that the 4×IC₅₀ 3′UTRMYC1-18 treated group had the best survival outcome with only one mouse dying from the group (FIG. 49). The next best outcome was shown for the 3′UTRMYC1-18 and olaparib treated group (FIG. 49). Following this was the 2×IC₅₀ group treated and then the IC₅₀ treated group (FIG. 49). The 4×IC₅₀ 3′UTRMYC1-18, the 2×IC₅₀ 3′UTRMYC1-18 and the combination treated group achieved a greater than 14 days survival outcome compared to the control. The IC₅₀ 3′UTRMYC1-18 treated group showed a 10 day survival outcome compared to the control. The log Rank (Mantel-Cox) test shows that the treated groups had a very significant survival outcomes (****P<0.0001) compared to the control group.

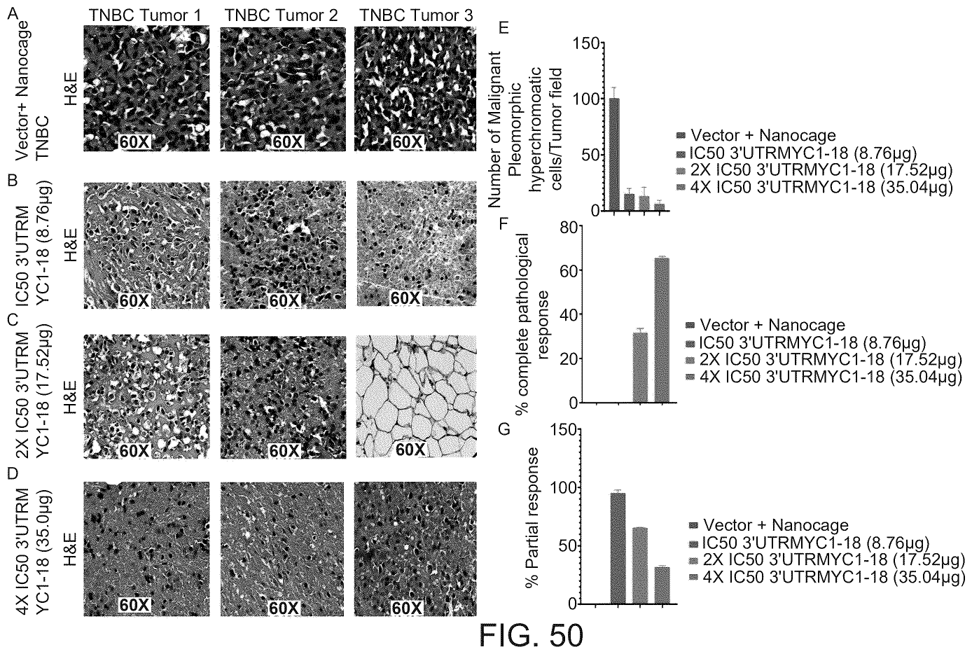

3′UTRMYC1-18 achieved in vivo tumor dose dependent complete pathological response and partial response in TNBC in a titratable manner. To validate the dose dependent inhibition in vivo, we performed H&E staining of the tumors from: 1) the vector+nanocage treated group, 2) the IC₅₀ 3′UTRMYC1-18 (8 0.76 μg) treated group, 3) the 2×IC₅₀ 3′UTRMYC1-18 (8.76 μg) treated group, and 4) the 4×IC₅₀ 3′UTRMYC1-18 (35.0 μg) treated group (FIG. 50A-E). We found that 3′UTRMYC1-18 achieved inhibition of the malignant pleomorphic hyperchromatic TNBC cells in a dose dependent manner with the highest inhibition these malignant cancer cells in the 4×IC₅₀ 3′UTRMYC1-18 treatment group (35.0 μg) (FIG. 50A-D). These results were quantified as shown in FIG. 50E. Moreover, the 4×IC₅₀ 3′UTRMYC1-18 (35.0 μg) treated mice achieved 75% complete pathological response (FIG. 50F), and the 2×IC₅₀ treated group achieved 38% complete pathological response. The IC₅₀ treated mice achieved 90% partial response, the 2×IC₅₀ treated mice achieved 60% partials response and the 4×IC₅₀ treated mice achieved 33% partial response (FIG. 50G).

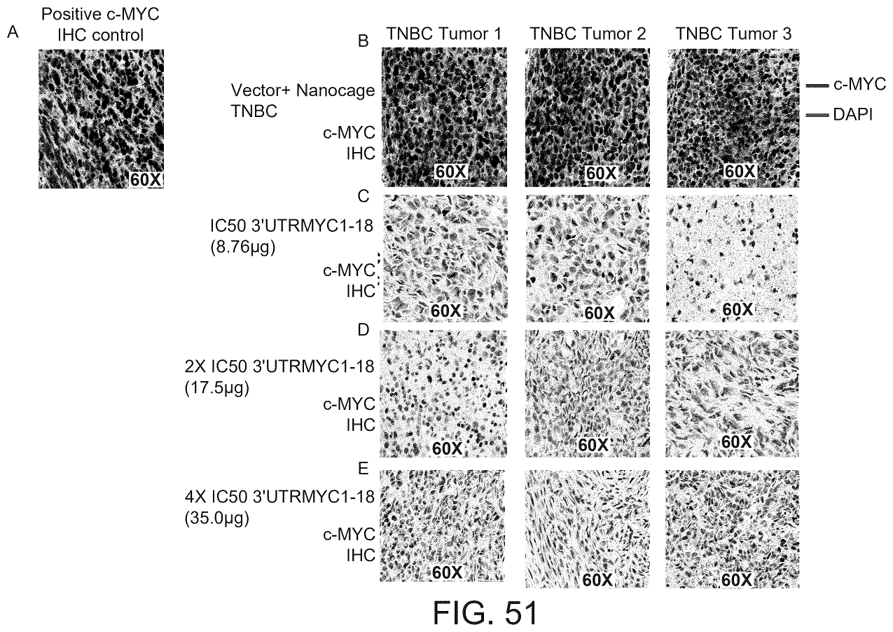

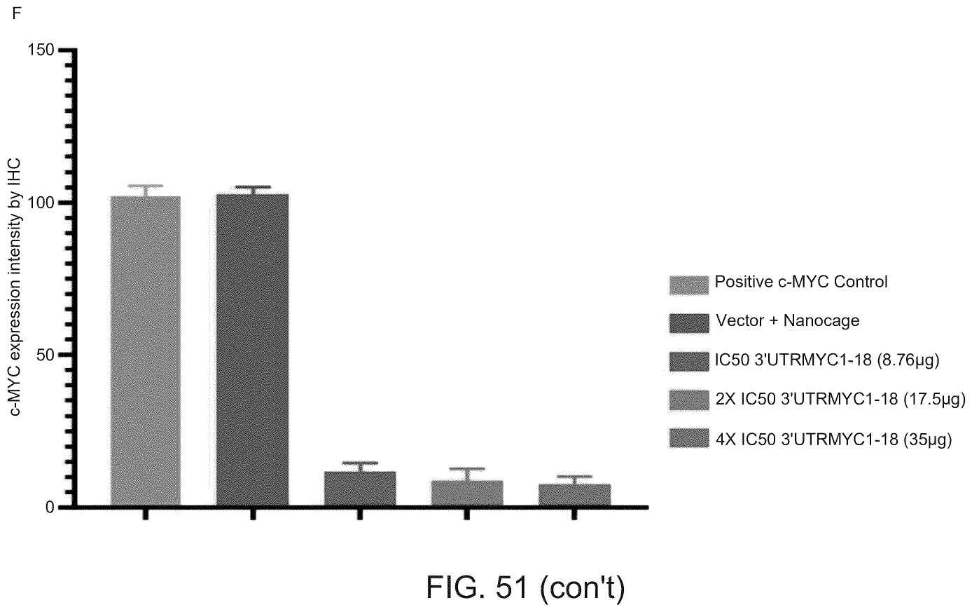

3′UTRMYC1-18 achieved on target c-MYC tumor inhibition in vivo in a dose dependent titratable manner. To validate in vivo that 3′UTRMYC1-18 destabilized and inhibited the oncogenic c-MYC in vivo in dose dependent titratable manner. We performed c-MYC IHC staining of the tumors from the positive c-MYC control tumor tissue, vector+nanocage treated group, the IC₅₀ 3′UTRMYC1-18 (8.76 μg) treated group, the 2×IC₅₀ 3′UTRMYC1-18 (8.76 μg) treated group, and the 4×IC₅₀ 3′UTRMYC1-18 (35.0 μg) treated group. We found that 3′UTRMYC1-18 inhibited c-MYC in a dose dependent manner (FIG. 51A-E). These results were quantified as shown in FIG. 51F. The highest inhibition of c-MYC was seen for 4×IC₅₀ 3′UTRMYC1-18 (35.0 μg) followed by 2×IC₅₀ and the IC₅₀ (FIG. 51E-C).

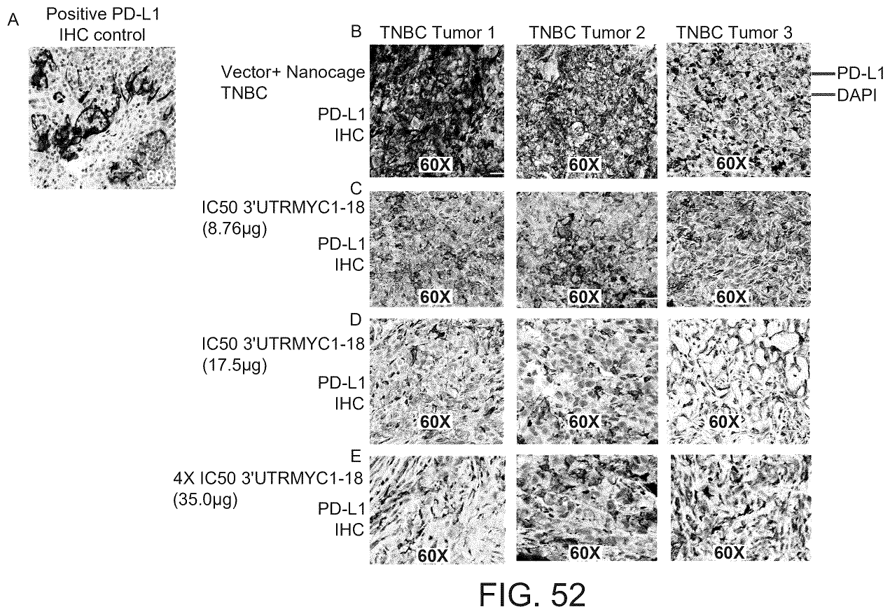

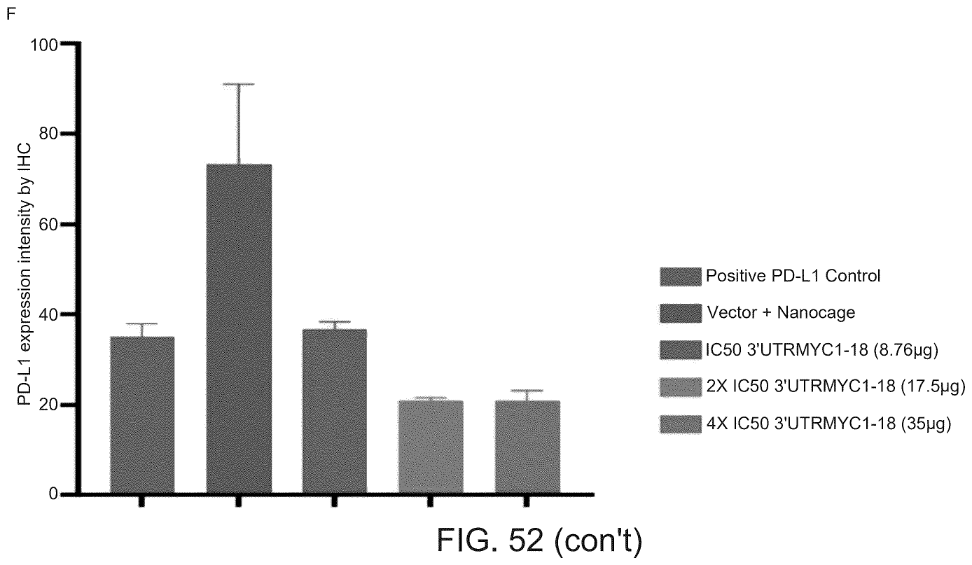

The dose dependent c-MYC inhibition in TNBC leads to dose dependent down regulation of the PD-L1 in vivo. c-MYC is known to bind PD-L1 to regulate this promoter. We and others have demonstrated that the downregulation of the c-MYC leads to the downregulation of the PD-L1. To revalidate this in the present experiments, we performed an TIC staining of the PD-L1 in; 1) the vector+nanocage treated group, 2) the IC₅₀ 3′UTRMYC1-18 (8.76 μg) treated group, 3) the 2×IC₅₀ 3′UTRMYC1-18 (8.76 μg) treated group, and 4) the 4×IC₅₀ 3′UTRMYC1-18 (35.0 μg) treated group. The positive control for these experiments was PD-L1 TIC staining from human tonsil (FIG. 52A). We found that loss of the c-MYC in dose dependent manner led to the down regulation of the PD-L1 in a dose dependent manner (FIG. 52B-F). The 4× and 2×IC₅₀ 3′UTRMYC1-18 treatments downregulated the PD-L1 the most followed by the IC₅₀ treated group. All data were quantified as shown in FIG. 52 F.

3′UTRMYC1-18 inhibits lung metastasis in a titratable dose dependent manner. We sought to confirm that 3′UTRMYC1-18 inhibited lung metastasis. We performed full necropsy of the lungs of the tumor bearing mice treated with 3′UTRMYC1-18 in a dose dependent manner as well as the controls and combination with olaparib (FIG. 53). We found that the 4×IC₅₀ 3′UTRMYC1-18+nanocage (35 μg) treatment inhibited lung metastasis the most (75%), followed by the combination of the IC₅₀ 3′UTRMYC1-18 (8.76 μg) and IC₅₀ olaparib (9.5 μg) treatment, and then followed by the 2×IC₅₀ 3′UTRMYC1-18 (17.5 μg) treatment (FIG. 53). The lowest inhibition was seen for the IC₅₀ 3′UTRMYC1-18 (8.76 μg) treatment (FIG. 53).

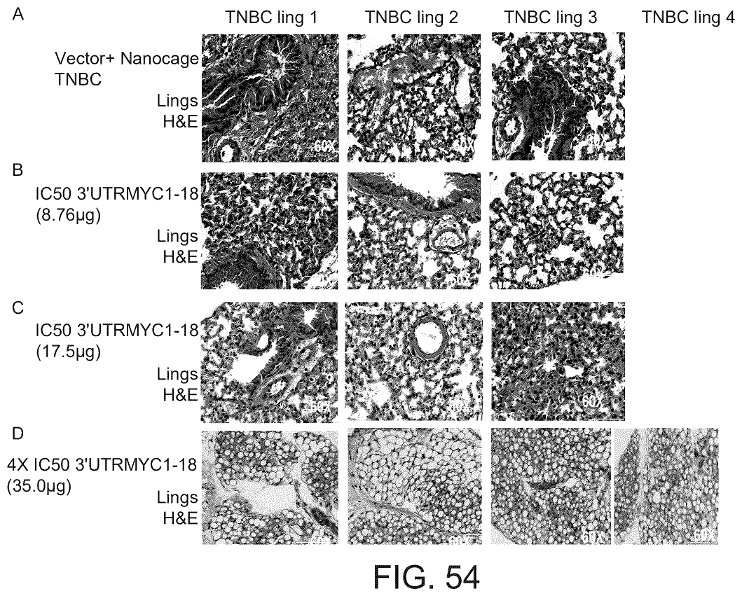

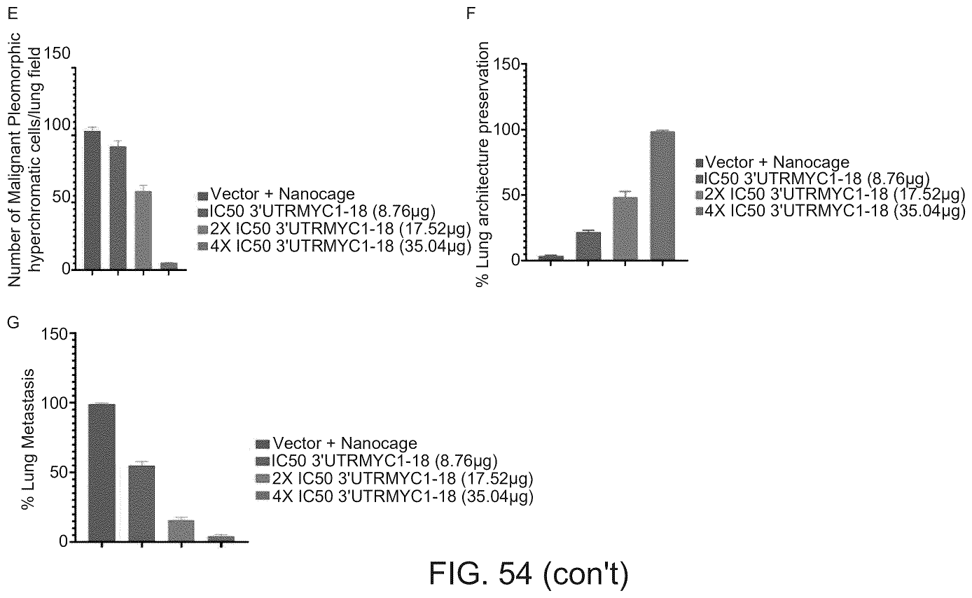

To validate lung metastatic tumor inhibition in vivo by 3′UTRMYC1-18 in dose dependent treatment, we performed a H&E staining of the lungs from: 1) the vector+nanocage treated group, 2) the IC₅₀ 3′UTRMYC1-18 (8.76 μg) treated group, 3) the 2×IC₅₀ 3′UTRMYC1-18 (8.76 μg) treated group, and 4) the 4×IC₅₀ 3′UTRMYC1-18 (35.0 μg) treated group (FIG. 54A-D). Strikingly, we found that 4×IC₅₀ treatment inhibited lung tumors up to 98% (FIG. 54D). 4×IC₅₀ 3′UTRMYC1-18 treatment also inhibited 98% of the malignant pleomorphic hyperchromatic tumor tissues in the lungs (FIG. 54E). In addition, 4×IC₅₀ 3′UTRMYC1-18 treatment preserved 98% of the lung architecture and inhibited 98% of the metastasis (FIG. 54G). The 2×IC₅₀ treatment achieved 50% inhibition of the malignant pleomorphic hyperchromatic cells and preserved 50% of the lung parenchyma with 70% inhibition of lung metastasis (FIG. 54C-G).

3′UTRMYC1-18 achieved the dose dependent lung metastasis inhibition through dose dependent inhibition of c-MYC. We next sought to confirm that c-MYC downregulation was the basis for the inhibition of the lung metastasis and preservation of the lung architecture. We performed c-MYC IHC staining of the lungs from: 1) the vector+nanocage treated group, 2) the IC₅₀ 3′UTRMYC1-18 (8.76 μg) treated group, 3) the 2×IC₅₀ 3′UTRMYC1-18 (8.76 μg) treated group, and 4) the 4×IC₅₀ 3′UTRMYC1-18 (35.0 μg) treated group (FIG. 55A-F). We found that 3′UTRMYC1-18 inhibited the c-MYC in dose dependent manner with the 4×IC₅₀ dose achieving the most inhibition of the c-MYC followed by the 2×IC₅₀ dose and the IC₅₀ dose (FIG. 55B-E). The results were quantified as shown in FIG. 55F.

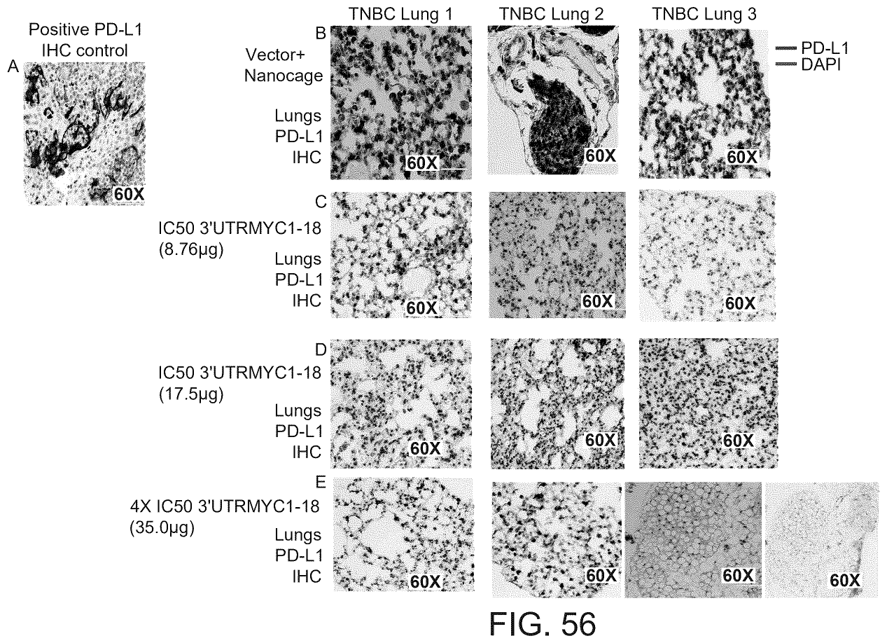

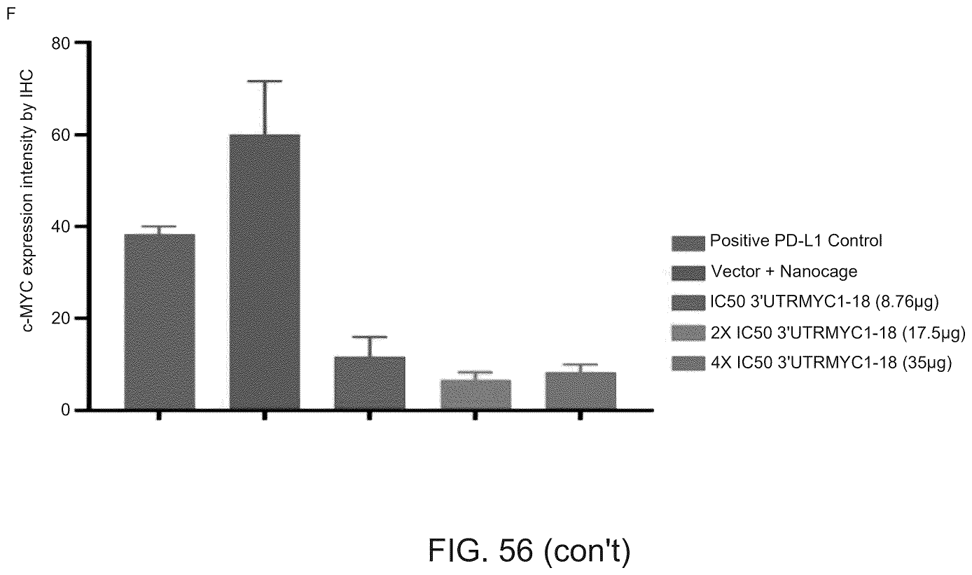

The dose dependent c-MYC in vivo inhibition in lungs leads to dose dependent down regulation PD-L1 in the lungs. We found that the dose dependent down regulation of c-MYC in lung tissues led to the dose dependent down regulation of the PD-L1 in the same tissues (FIG. 56A-F). 4×IC₅₀ 3′UTRMYC1-18 treatment achieved the greatest inhibition of PD-L1 expression followed by 2×IC₅₀ treatment and IC₅₀ treatment. The results were quantified as shown in FIG. 56F.

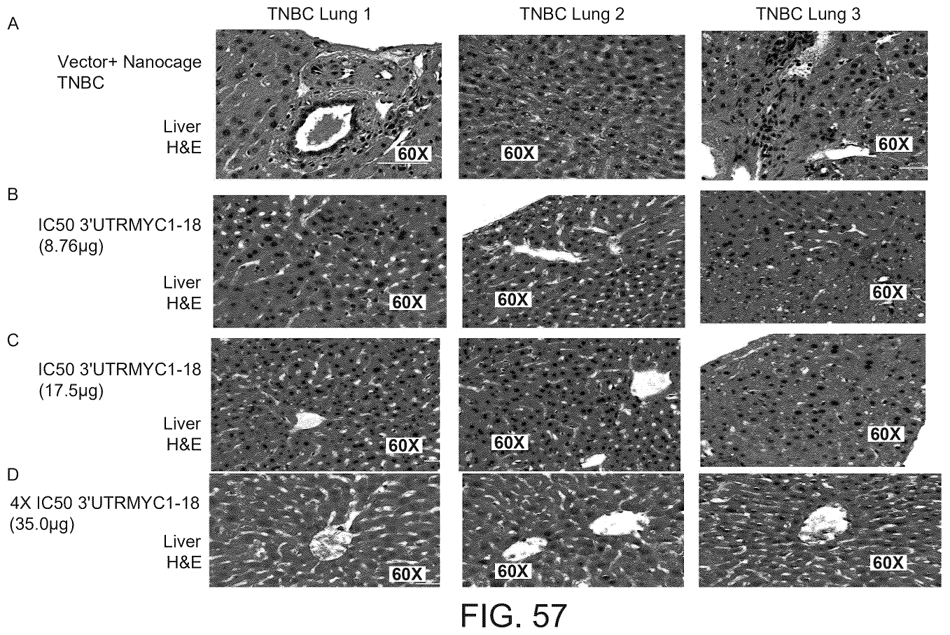

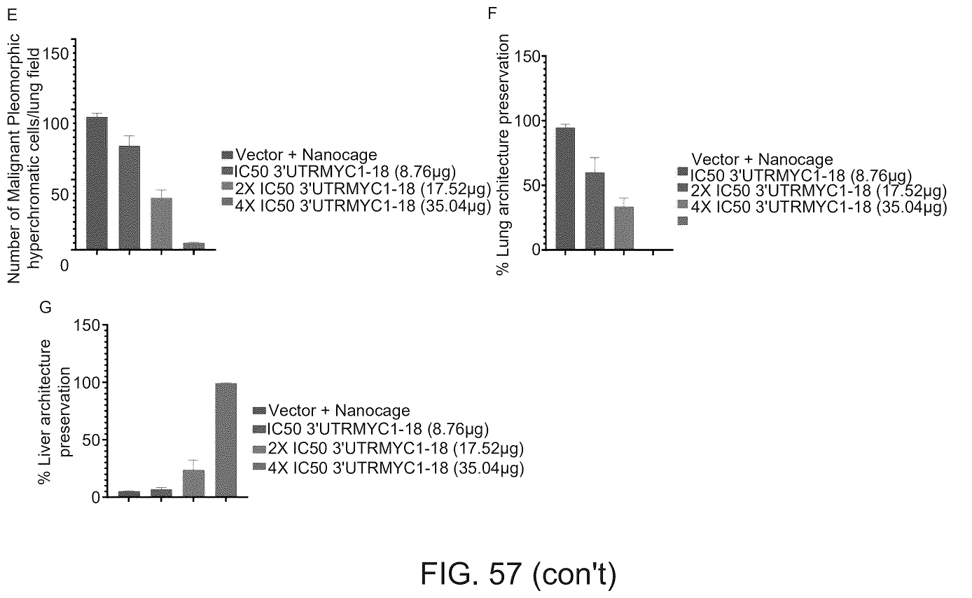

3′UTRMYC1-18 inhibits liver metastasis in dose dependent manner. We examined the livers of the treated animals and the controls to see if there would be dose dependent inhibition of metastasis. We found that 3′UTRMYC1-18 inhibited liver metastasis in a dose dependent manner (FIG. 57A-G). Notably, 4×IC₅₀ treatment achieved complete degradation of cancerous malignant pleomorphic hyperchromatic cells in the liver tissues, the complete inhibition of liver metastasis, and the preservation of the liver architecture (FIG. 57D-G). 2×IC₅₀ treatment achieved less than 50% liver metastasis inhibition and preservation of the liver parenchyma as well as inhibition of the malignant pleomorphic hyperchromatic cancerous cells to the liver. 1×IC₅₀ treatment was the least effective. The IC₅₀ dose achieved less than 10% inhibition of the malignant pleomorphic hyperchromatic cells (FIG. 57B, E), 20% inhibition of liver metastasis (FIG. 57B, F) and the liver architecture was not preserved in the IC₅₀ dose (FIG. 57 B, G).

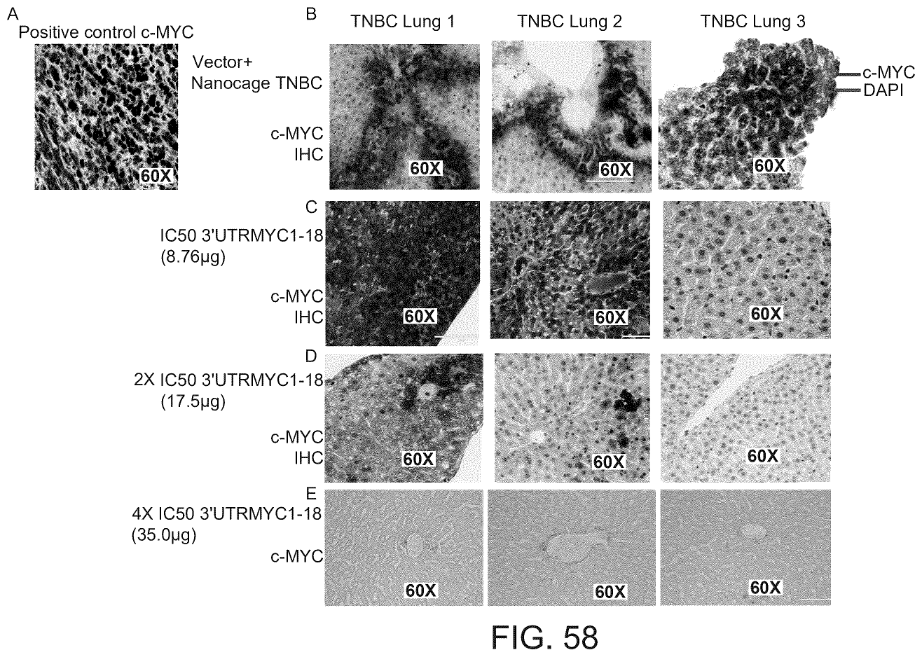

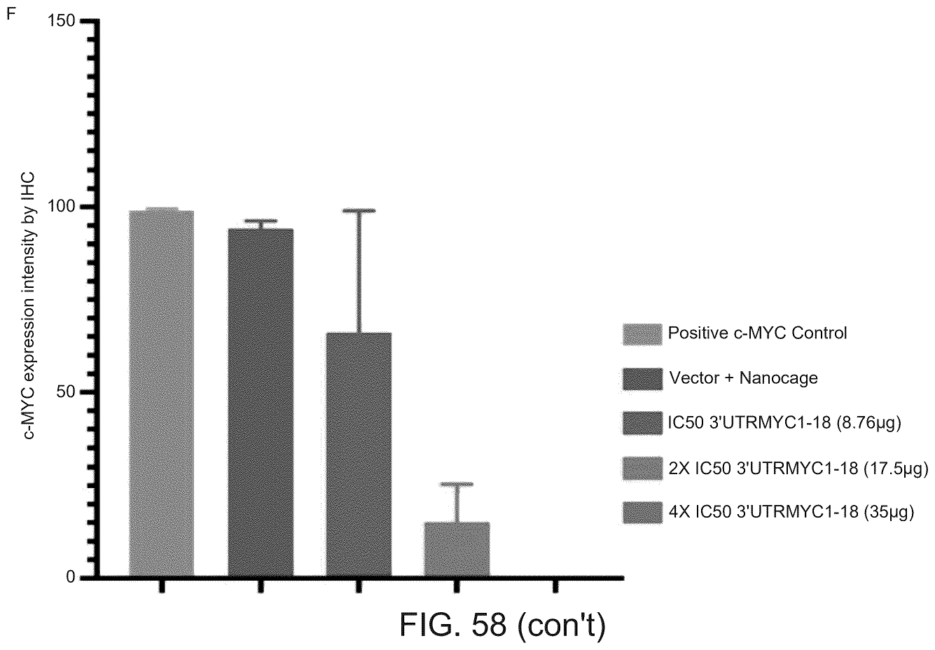

3′UTRMYC1-18 inhibits liver metastasis and the preservation of liver architecture through dose dependent down regulation of the c-MYC. We sought to confirm 3′UTRMYC1-18 dose dependent down regulation of c-MYC in the livers of the TNBC tumor bearing mice. We performed a c-MYC IHC staining of the livers from: 1) the vector+nanocage treated group, 2) the IC₅₀ 3′UTRMYC1-18 (8.76 μg) treated group, 3) the 2×IC₅₀ 3′UTRMYC1-18 (8.76 μg) treated group, and 4) the 4×IC₅₀ 3′UTRMYC1-18 (35.0 μg) treated group. We found that 3′UTRMYC1-18 inhibited c-MYC expression in the livers in a dose dependent manner. 4×IC₅₀ treatment was the most effective followed by the 2×IC₅₀ treatment (FIG. 58A-F). The IC₅₀ dose was the least effective in inhibiting MYC expression in the liver (FIG. 58C).

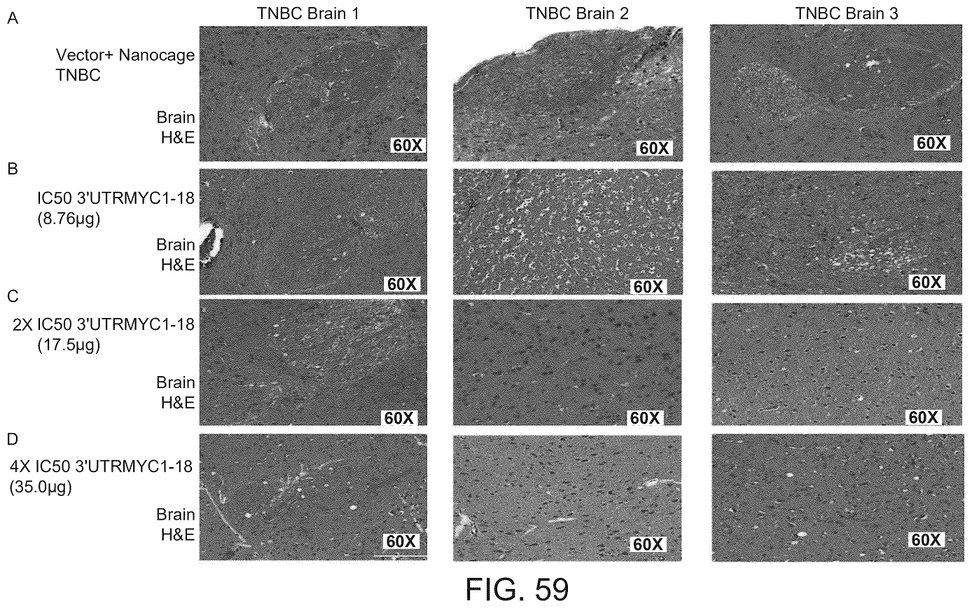

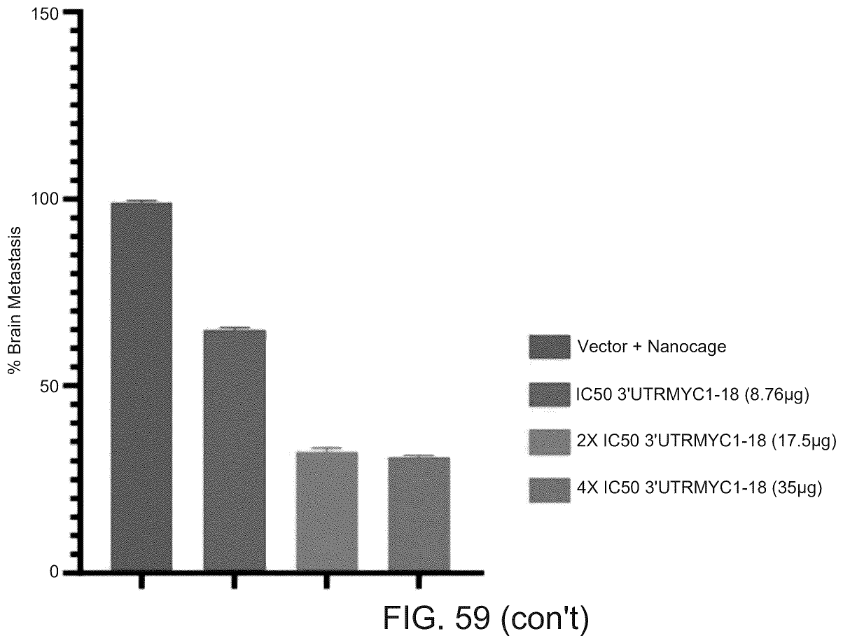

3′UTRMYC1-18 inhibits brain metastasis in a dose dependent manner. Triple negative breast cancers metastasize to the brain and such metastases are fatal. We assessed whether 3′UTRMYC1-18 inhibited brain metastasis in the dose dependent manner. We stained the brain tissue of the treated animals and the controls with H&E. The vector+nanocage treated group showed metastasis in 100% of the animals (3/3) (FIG. 59A, see arrow). The IC₅₀ 3′UTRMYC1-18 treated group showed metastasis in 66% of the animals (2/3) (FIG. 59B, see arrow). The 2×IC₅₀ and 4×IC₅₀ treated groups showed metastasis in 33% of the animals (1/3) (FIG. 59C-D, see arrow). The number of brain metastases was quantified as shown in FIG. 59F. Taken together, these results are highly informative and clearly point to 3′UTRMYC1-18+nanocage passing through the blood brain barrier and inhibiting metastasis in a dose dependent manner.

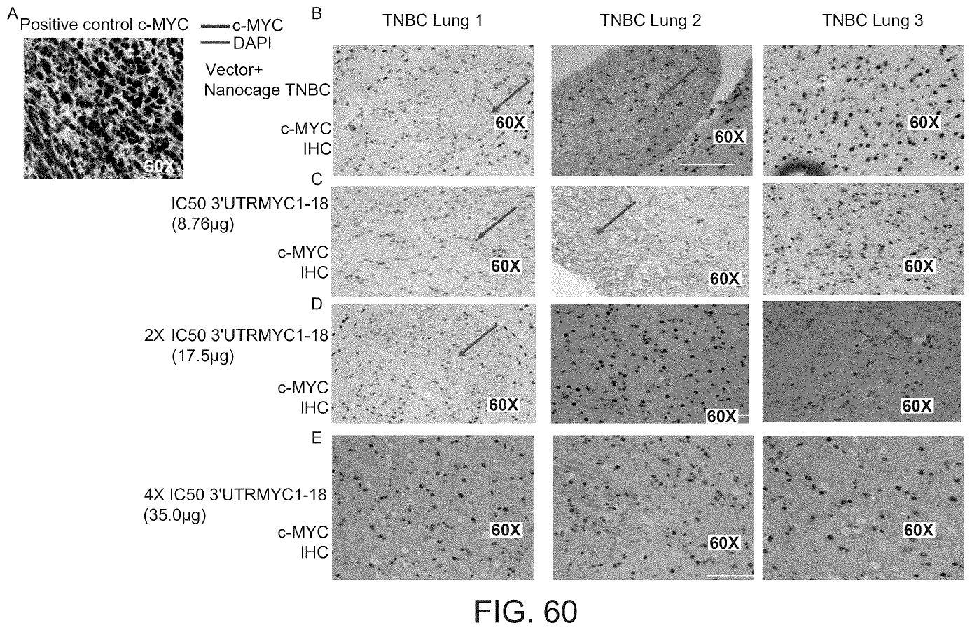

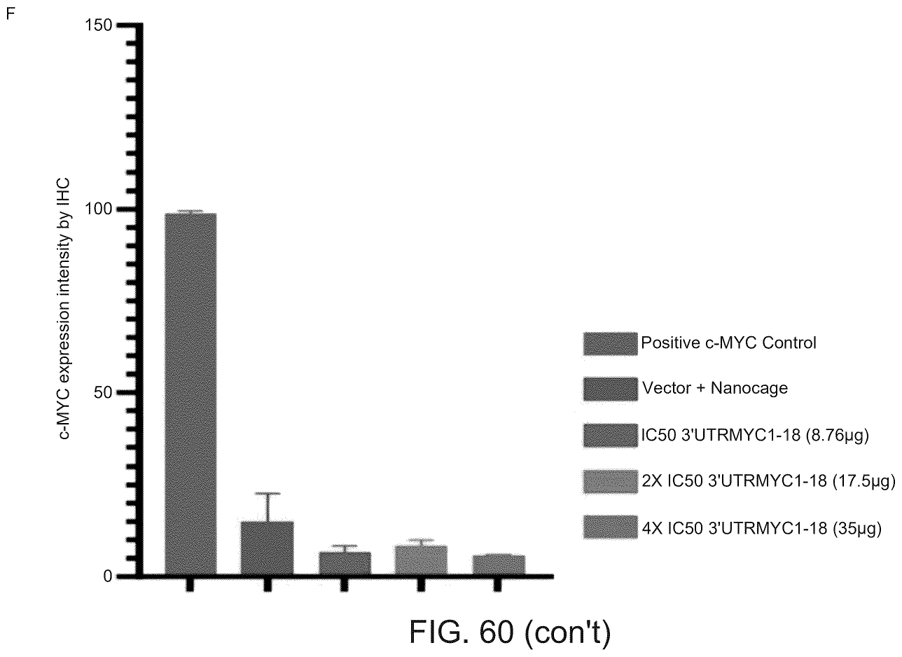

The 4×IC₅₀ 3′UTRMYC1-18 (35 μg) inhibits c-MYC expression in brain metastasis. We extended analysis into whether we inhibited brain metastasis. We performed an IHC staining for c-MYC in the brains of the treated groups and the controls. We found a low level of c-MYC expression in the brain (FIG. 60A-F). We found that 4×IC₅₀ treatment produced the greatest inhibition of c-MYC expression (FIG. 60E).

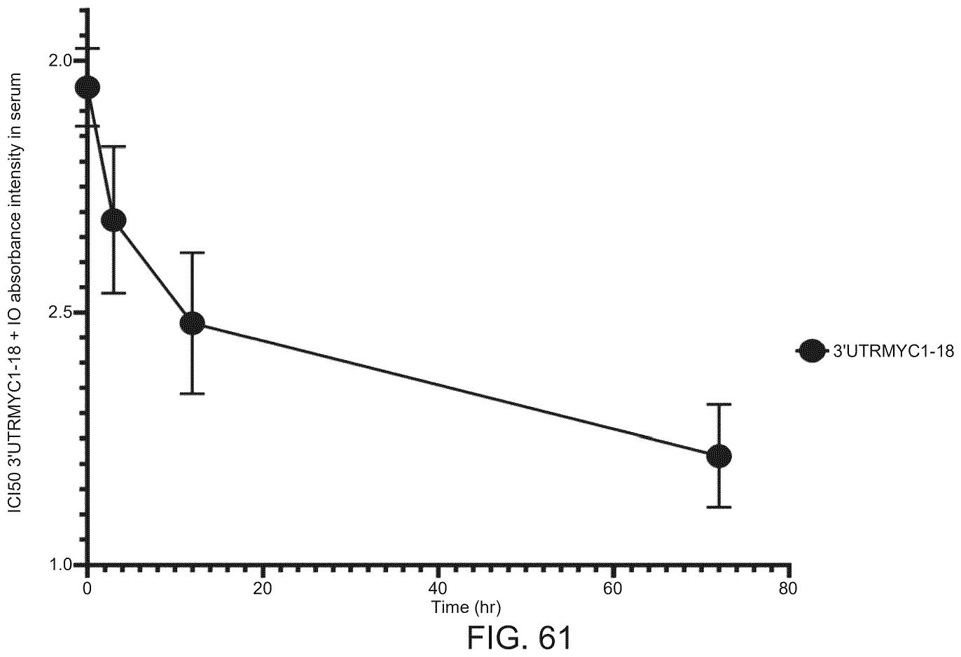

Pharmacokinetics of 3′UTRMYC1-18 in vivo in the mice bearing tumors. We sought to understand the pharmacokinetics of 3′UTRMYC1-18 in vivo in tumor bearing mice. To assess this, we administered the mRNA destabilizing drug the IC₅₀ 3′UTRMYC1-18+IO nanocage (8.7 μg) intravenously and collected the blood for time point 0, 3 hrs, 6 hrs, and then 1 week. We collected the serum and then performed detection of the iron oxide using ELISA based assay. We read the absorbance intensity of the various time points, and obtained the data presented in FIG. 61. The results indicate that the drug has a relatively stable long half-life in serum, this being greater than 24 hr.

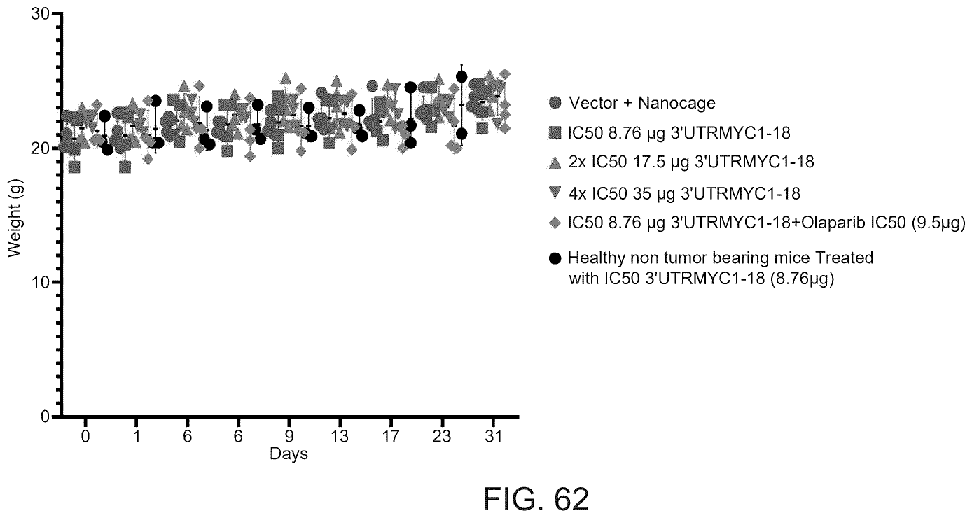

3′UTRMYC1-18 does not affect the weight of tumor bearing and healthy animals treated with it. Cachexia is one of the side effects of cancer chemotherapies. We have found here that 3′UTRMYC1-18 does not affect the weight of the tumor bearing animals treated with the novel mRNA destabilizing drug. We determined that 3′UTRMYC1-18 does not cause weight loss for tumor bearing mice treated with the drug and healthy non-tumor bearing mice treated with the drug (FIG. 62).

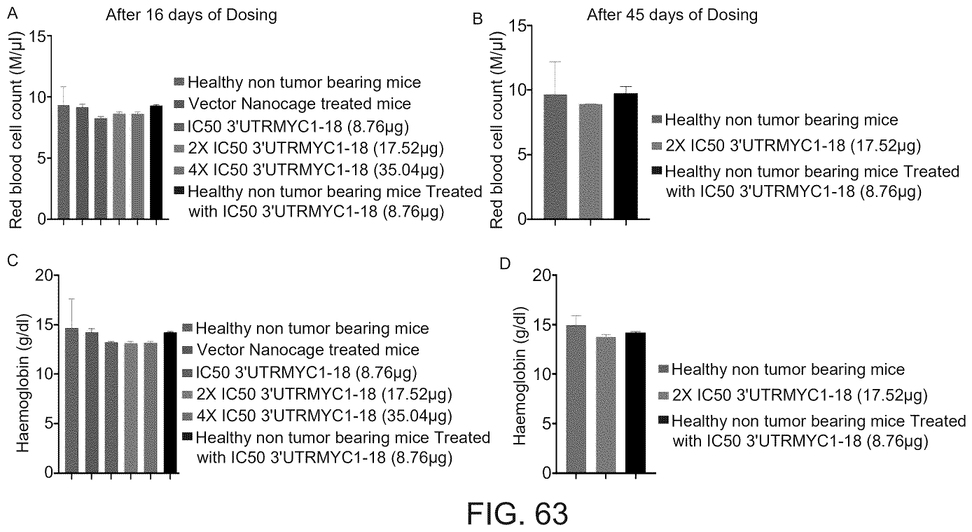

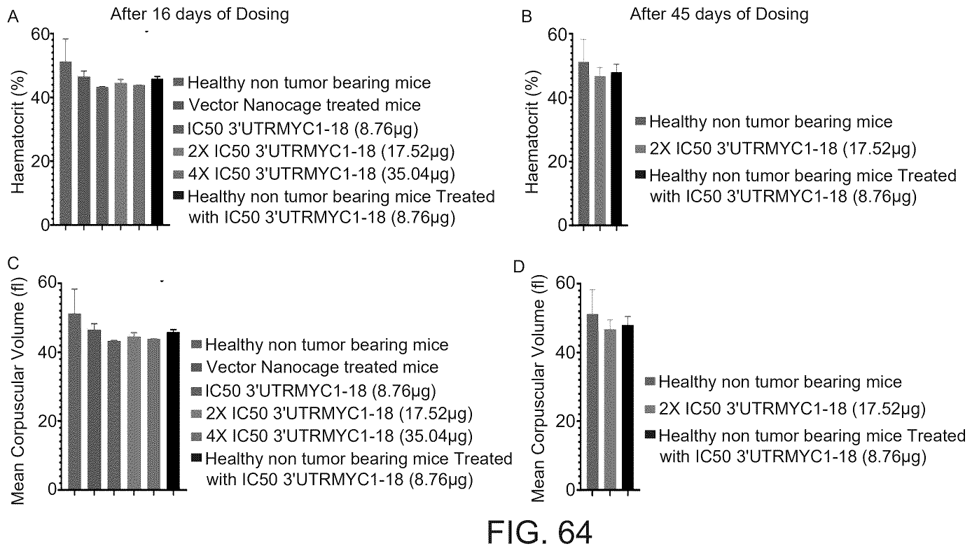

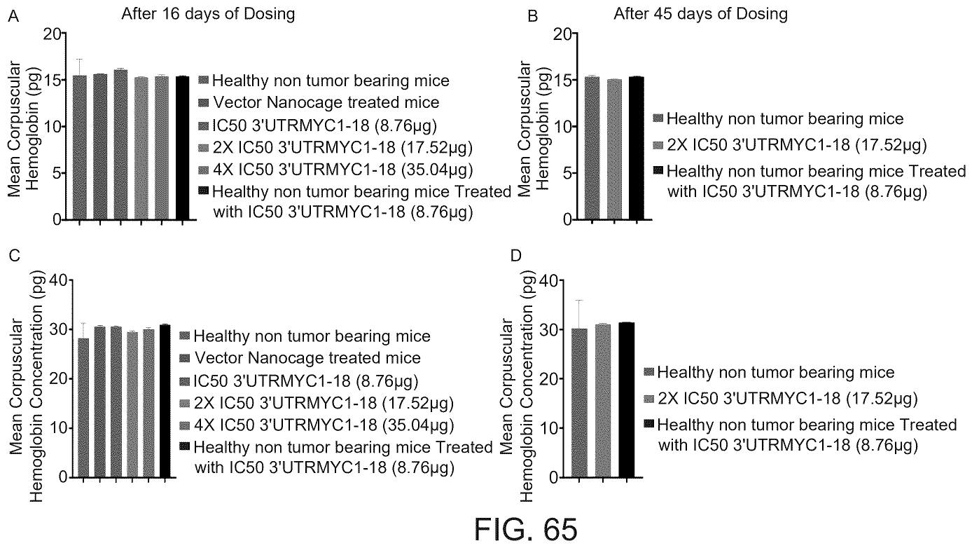

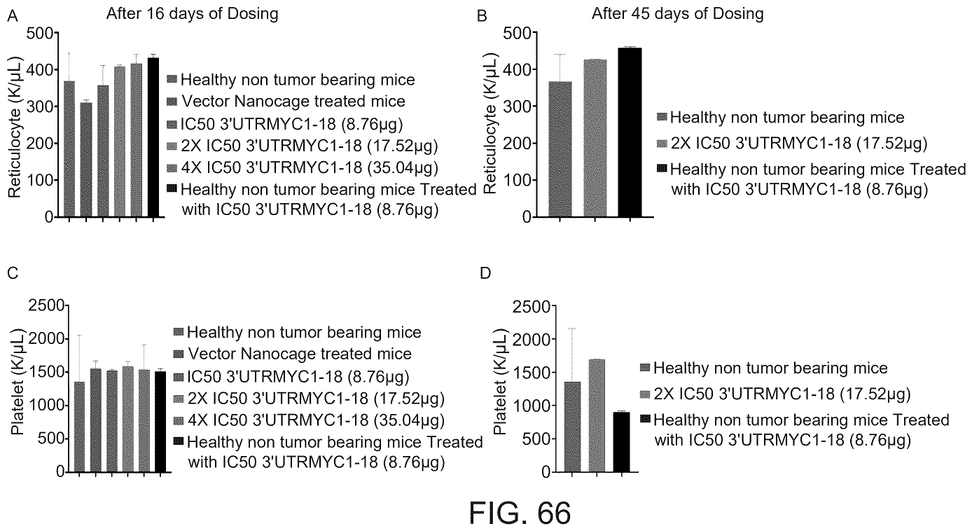

The dose dependent 3′UTRMYC1-18 in vivo treatment is safe for the blood cells, electrolyte, liver, pancreatic and kidney functions both in short and long-term period of treatment of the tumor bearing mice as well as in the healthy non-tumor bearing mice. To understand the toxicity and safety profile of 3′UTRMYC1-18, we assessed both short- and long-term dosing which were 16 days and 45 days, respectively. We assayed red blood cell counts and hemoglobin concentration. We found no changes in the red blood cell count and hemoglobin concentration both on the short- and long-term administration (FIG. 63-66).

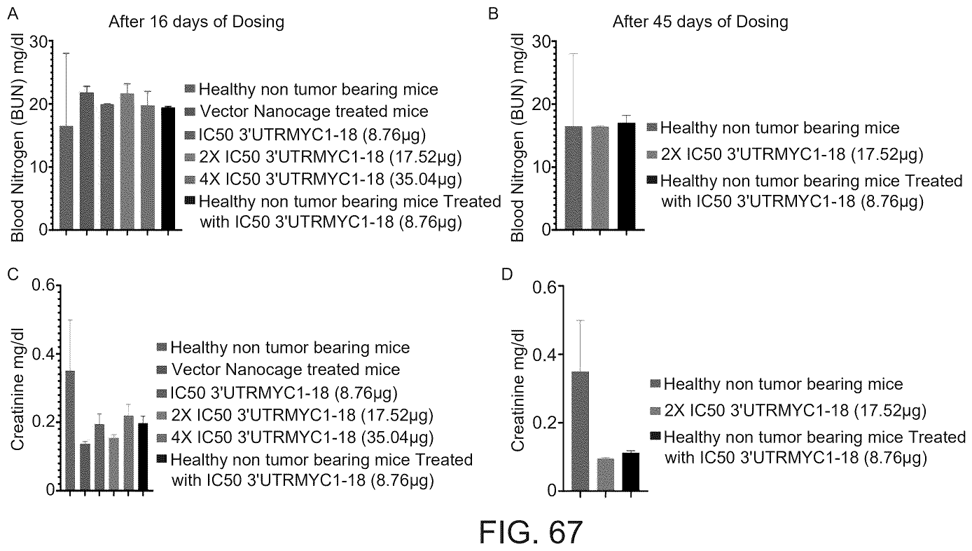

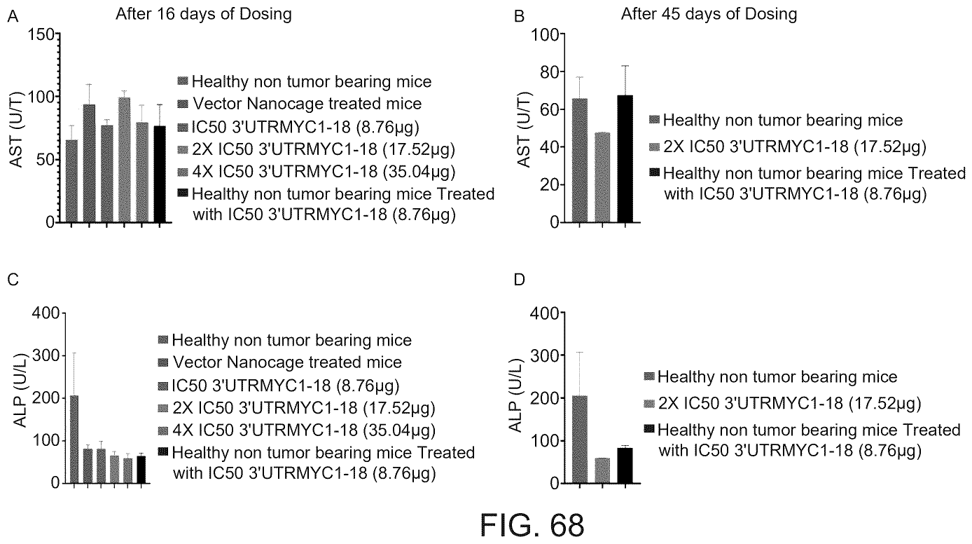

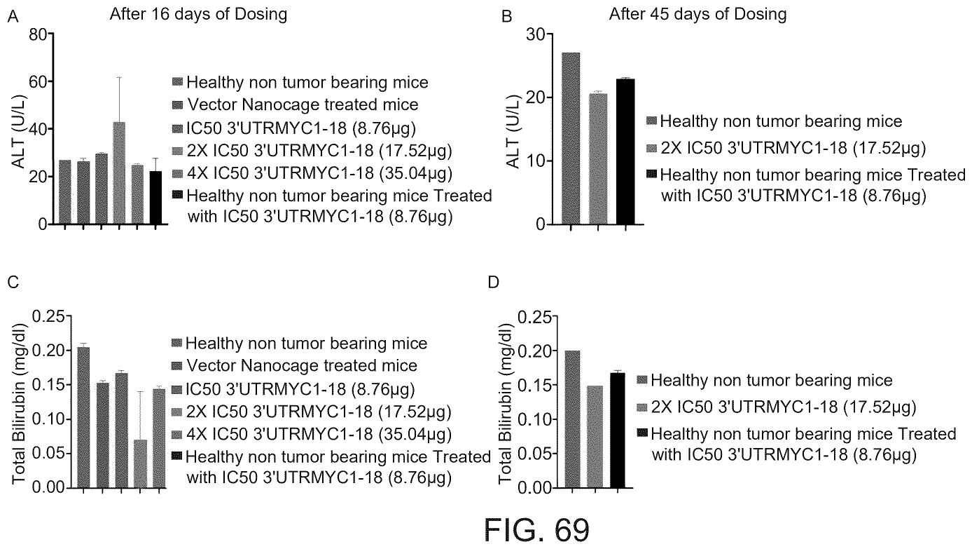

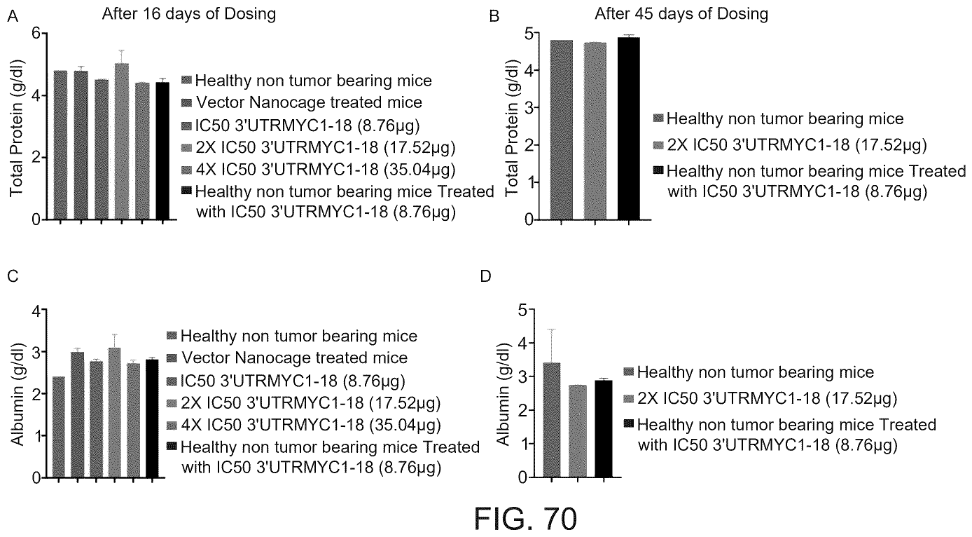

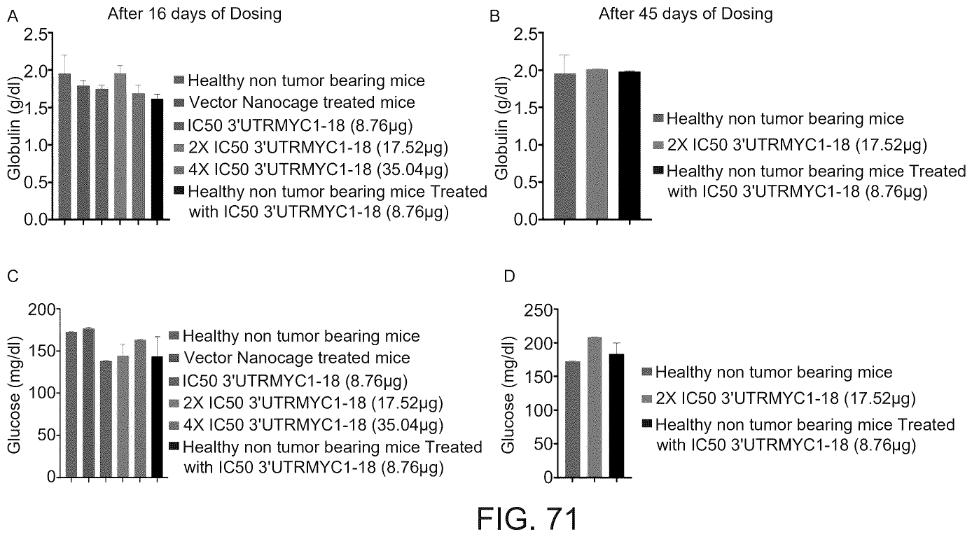

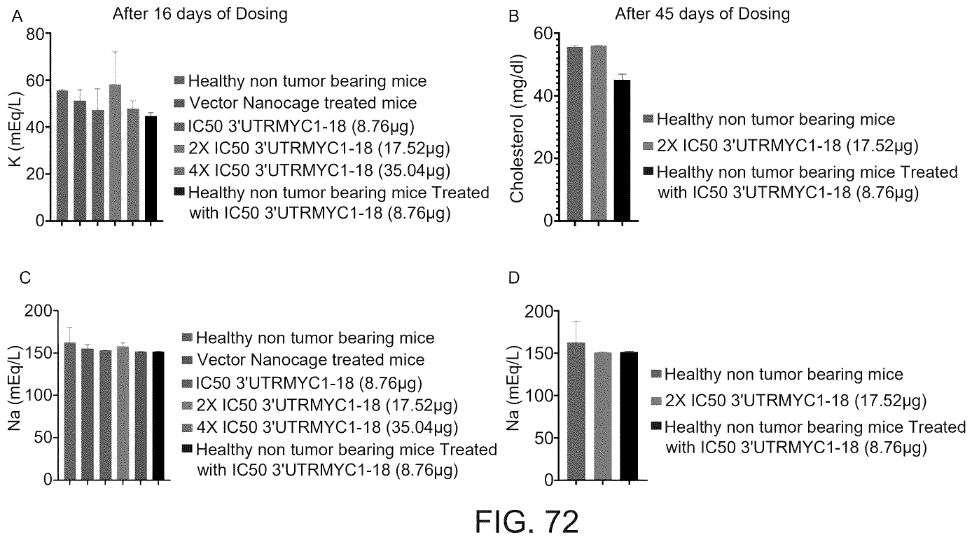

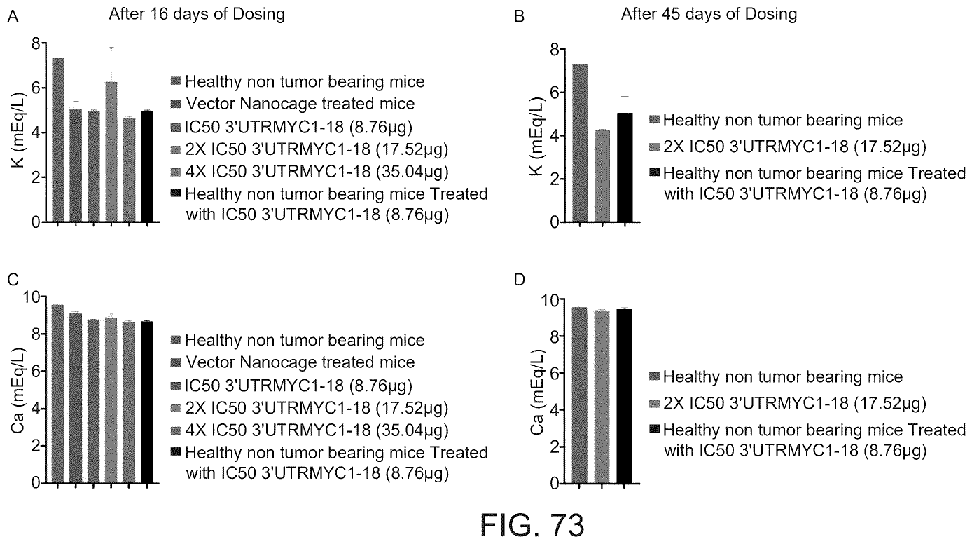

We next assessed the safety of 3′UTRMYC1-18 in vivo treatment for other blood markers, electrolytes, and liver, pancreatic, and kidney functions in short and long-term periods for tumor bearing mice as well as healthy non tumor bearing mice. Short- and long-term dosing protocols were carried out for 16 days and 45 days, respectively. We gauged profiles for blood urea nitrogen levels and creatinine levels (FIG. 67). We gauged profiles for AST (aspartate aminotransferase) levels and ALP (alkaline phosphatase) levels (FIG. 68). We gauged profiles for ALT (alanine aminotransferase) levels and total bilirubin levels (FIG. 68). We gauged profiles for total protein levels and albumin levels (FIG. 69). We gauged profiles for globulin levels and glucose levels (FIG. 70). We gauged profiles for cholesterol levels and sodium levels (FIG. 71). We gauged profiles for potassium levels and calcium levels (FIG. 72). In sum, we found no significant changes in any of the safety parameters tested from short- and long-term administration of 3′UTRMYC1-18.

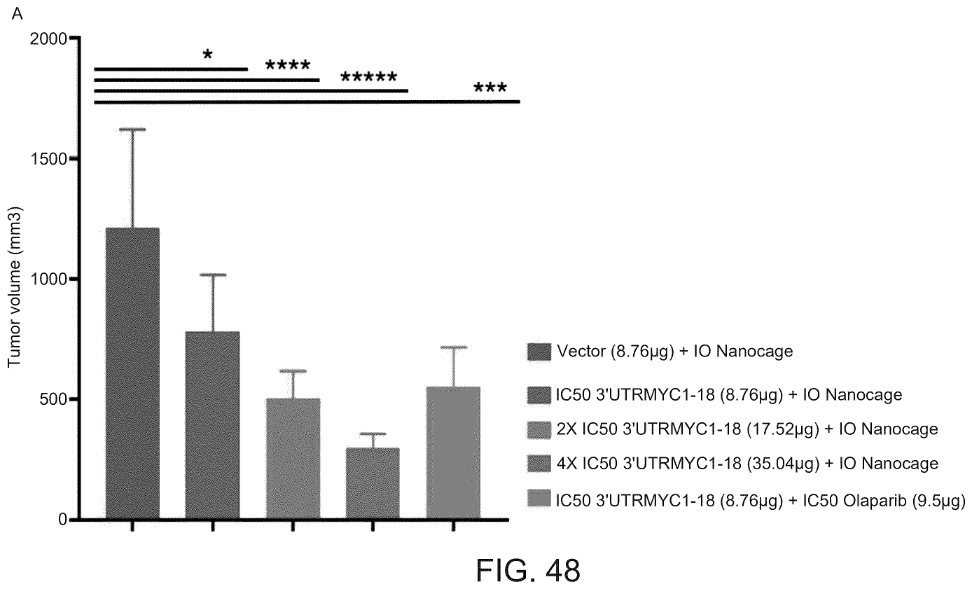

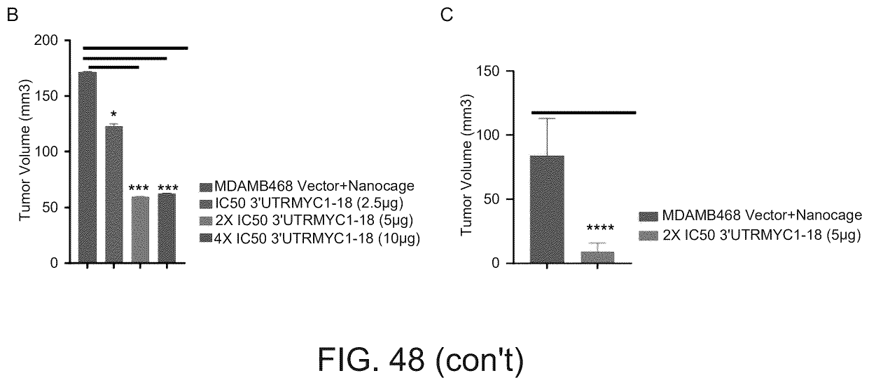

Daily tumor volume measurement and recording for MDAMB468. To obtain the daily tumor volumes, we used callipers and measured tumor length and width. Using the formula (½XLXWXW), we calculated the tumor volume. We found that the drug inhibited the tumor in a dose dependent manner 4×IC₅₀=2×IC₅₀>IC₅₀ (FIG. 47C). We showed by tumor images that the c-MYC mRNA destabilizing drug, 3′UTRMYC1-18, inhibited the MDAMB468 TNBC cells in a titratable dose dependent manner (FIG. 47D-E; FIG. 48B-C).

### Example 3C: Discussion

3′UTRMYC1-18 shows therapeutic efficacy in diverse breast cancers including TNBCs from African descent and Caucasian descent patients. 3′UTRMYC1-18 acts in a dose dependent manner and is safe and well tolerated. 3′UTRMYC1-18 is highly effective in achieving on target dose dependent down regulation of c-MYC and PD-L1 and inhibits lung and liver and brain metastases in a dose dependent manner. It is considered that 3′UTRMYC1-18 will find utility in the treatment of a wide range of breast cancer patients, including any ethnicities.

### Example 4: The In Vivo Destabilization and Downregulation of the Oncogenic c-MYC mRNA Inhibits Lethal Metastatic c-MYC Driven Pancreatic Cancers

Overview: Pancreatic ductal carcinoma is the most common form of pancreatic cancer and is the deadliest form with an 11% survival rate. There is currently no cure. The mainstay drug used for pancreatic cancer is gemcitabine, capecitabine or FOLFIRINOX as first line therapy for pancreatic cancer. After 21 months, chemoresistance starts driven by the oncogenic c-MYC signal. This is a significant clinical and cancer biology challenge. The c-MYC oncogene has been shown to be over expressed in the 43.1% and the 31.6% primary and metastatic pancreatic cancer respectively and is the primary driver of the neoplastic changes and progression of pancreatic cancer metastasis. Here, we report the in vivo down regulation and inhibition of the metastatic c-MYC expressing lethal pancreatic cancer by the mRNA destabilizing drug 3′UTRMYC1-18. The drug achieved on-target in vivo c-MYC dose-dependent downregulation with complete pathological response, inhibition of liver, lung and brain metastasis with significant survival outcome and is safe and well tolerated. We show that the therapeutic efficacy of the MYC mRNA destabilizing drug involves down regulation of c-MYC-PD-L1.

### Example 4A: Materials and Methods

The development of the 3′UTRMYCM1-18 mRNA destabilizing drug: We have previously described the c-MYC mRNA destabilizing drug (13c). Briefly, we discovered the stable mRNA poly U sequences on the 3′UTR of c-MYC and engineered them to unstable forms driven under the control of mRNA de-capping promoter. The mRNA destabilizing drug directly binds their target mRNA recognition site either in-frame or at the 3′UTR or both and triggers the stalling of ribosome. This stalling is sensed by PELO-EXOSC4-RPL3 complex, which triggers the degradation of the target transcript. This does not affect normal healthy cells because such cells do not differentially express EXOSC4, PELO and RPL3.

Cell Culture: We obtained the following pancreatic cancer cells the PSN1, MIA-Paca2 and the PANC-1 from ATCC. The PSN1 was grown in RPMI media supplemented with antibiotics/antimycotics before use. The PANC-1 and MIA-Paca-2 were grown in DMEM media supplemented with antibiotics/anti-mycotic before use. 10% FBS were added to the media. The cells are authenticated by ATCC by STR sequencing and we regularly tested for mycoplasma before use.

Dose dependent IC₅₀ determination and comparison with standard of care drugs: We determined the IC₅₀ of the MYC mRNA destabilizing drug 3′UTRMYC1-18 by titrating the drug in dose dependent manner in a head-to-head comparison with the standard of care drugs. This included olaparib, paclitaxel, cisplatin, actinomycin D, bevacizumab, cyclophosphamide, MYCi975, and osimertinib. All drugs were obtained from Selleckchem (USA). The doses used ranged from 2.5 μg to 40 μg. Serial dilutions of the drugs were made. We seeded the cells at 5,000 cells per well in a 96 well plate. The cells were allowed to be attached for 24 hrs and then the drugs were added. We incubated the treated cells for 72 hrs. Using the CELLTITER-GLO (Promega G7570), we read the viability and normalized it to the controls and the data curve were fitted on the drug dose response chart in the GraphPad Prism (USA). From this, the IC₅₀ values were derived.

Quantitative Reverse Transcript PCR: We sought to quantify the dose dependent down regulation of c-MYC transcript in pancreatic cancer. We treated the MIA-Paca-2 cells with dose dependent concentration of 3′UTRMYC1-18. We extracted the RNA using the Qiagen RNeasy kit (Cat No. 74104). The RNA was stored at −80° C. before use. To reverse transcribe the RNA, we used the Superscript IV reverse transcriptase kit (Cat No. 18090200) to make cDNA. We designed the qPCR primers targeting the exons of the MYC and GAPDH as housekeeping gene control. The primers used are set out in (13c). See, also, below. The delta CT was used to normalize the transcript expression.

| | | |
|---|---|---|
| c-MYC forward primer (qPCR) | GTCACACCCTTCTCCCTTCG | SEQ ID 158 |
| c-MYC reverse primer (qPCR) | CAGGTACAAGCTGGAGGTGG | SEQ ID 159 |
| GADPH forward primer (qPCR) | GCCTCACTCCTTTTGCAGAC | SEQ ID 160 |
| GADPH reverse primer (qPCR) | AAGTGGTCGTTGAGGGCAAT | SEQ ID 161 |

Iron oxide nanocage and 3′UTRMYC mRNA destabilizing drug complexation: The iron oxide nanocage and the MYC mRNA destabilizing drugs were complexed in a 1:1 (13c, 16c) ratio. The conjugate was incubated over night before use at 4° C. Subsequently, the drug conjugate was found to be stable at room temperature, 4° C. or −20° C. until use.

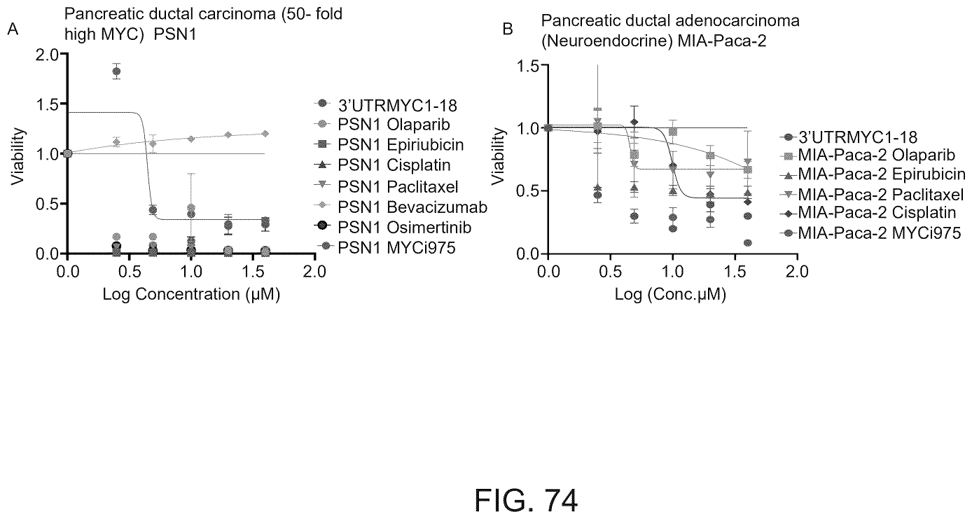

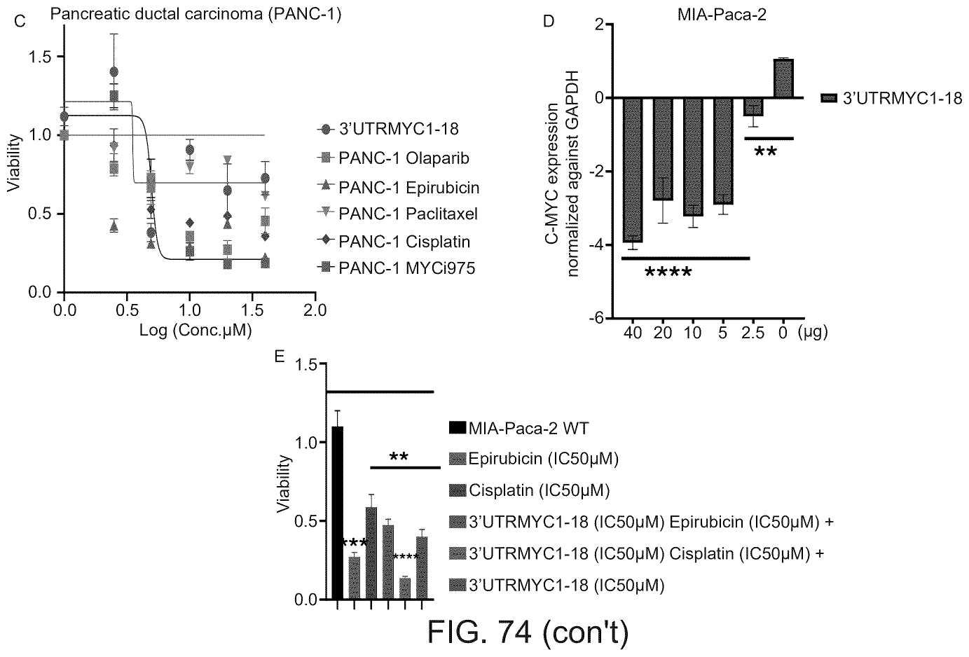

Animal studies—In vivo therapeutic efficacy of the c-MYC mRNA destabilizing drug in lethal pancreatic cancer: We obtained IACUC institutional approval from the CUNY institutional review board. We ordered 5 females and 3 males NSG mice from the Jackson laboratory. The mice were aged 5-8 weeks with weights of 15-21 g Once received, we allowed the mice to acclimatize according to the protocol. We implanted 10 million PSN1 cells into the flank of the mice. After 7 days post implantation, the tumor engrafted. On day 10, we randomized the animals into equal tumor volume and weight into the groups: 1) vector+nanocage group (N=4) and 2) 3×IC₅₀ 3′UTRMYC1-18+nanocage group (N=4). All mice had tumors and there were no excluded animals. We started treating the animals intravenously with the 3×IC₅₀ dose of the drugs 2× per week. This equates to 3.5 μg as determined from FIG. 74. On day 25, we collected the blood of the animals to assess for the safety profile. This included full blood count, electrolyte, liver, kidney, pancreatic and gall bladder function. The vector+nanocage treated tumor bearing mice died on day 26. The 3×IC₅₀ treated group lived until day 32.

Animal studies—Dose dependent inhibition of tumor volume by the MYC mRNA destabilizing drug in pancreatic cancer: We obtained 8 mice (7 females, 1 male) from the Jackson laboratory. The mice were aged 5-8 weeks and weighed 15-20 g. We allowed the animals to acclimatize. We implanted 10 million PSN1 cells into the flank. By day 7, the tumors were engrafted. We randomized the animals into 3 groups by day based on tumor size and weight. The groups included: 1) vector+nanocage treatment (N=2), 2) 6×IC₅₀ 21 μg treatment (N=3), 3) 9×IC₅₀ 31 μg treatment (N=3). Animals were weighed daily, body condition score and tumor volume measured with calipers as ½XLXWXW and recorded and documented. We dosed the animals 2× per week intravenously through the tail veins until day 28. By day 27, the vector+nanocage treated mice were dead. The 6×IC₅₀ treated group died on day 33. We dosed the 9×IC₅₀ 1× per week from day 28 to day 33 after which we stopped dosing. The mice in the 9×IC₅₀ group lived until day 55 and were euthanized. Euthanization was performed with CO₂. We recorded daily tumor volumes, weight, and body condition score. The Log Rank Mantel test was used to determine statistical significance between the in vivo treatment groups. The Kaplan Meier survival curve was used to determine survival differences between the various treatment groups and controls.

Safety profile analysis: To determine the safety of the c-MYC mRNA destabilizing drugs, we assessed blood cells, serum electrolyte, kidney, liver and pancreatic and the gall bladder function. We collected blood and serum and sent it to the Memorial Sloan Kettering Cancer Core Pathology laboratory. The laboratory ran the full blood count, lipid profile, liver enzymes, kidney function and electrolyte analysis. The reference was normal NSG mice. The analyst was blinded to the experimental details.

Necropsy: Upon death, we took the fresh carcasses, dissected them, and collected the tumors, lungs, kidney, livers, and brain. We rinsed the lungs in 1×PBS and took the images of the fresh organs. Subsequently, we collected fresh tissues of tumors and organs We froze these at −80° C. All tissues were then placed in 4% paraformaldehyde until being sent to the pathologist lab for embedding and H&E and IHC staining.

H&E staining of tumors and organs: For experiment #1, H&E images of the vector+nanocage and 3×IC₅₀ treated groups were obtained by the core pathology group of the Memorial Sloan Kettering Cancer Center. For experiment #2, the vector+nanocage samples and the 6×IC₅₀ and 9×IC₅₀ samples were sectioned by QC and H&E stained by JPD. The Images of the stained tissues were obtained using the EVOS FL microscope at 40× magnification. Quantification was performed in Image J (RRID:SCR_003070)(https://imagej.net/ij/) and in Graph Pad Prism (v10) RRID:SCR_002798.

TIC staining of MYC and PD-L1 of tumors and organs: We performed IHC staining against MYC and PD-L1 on the tumors and organs of the treated animals and the controls. We used the Abcam IHC protocol (see https://www.abcam.com/en-us/technical-resources/protocols/ihc-with-samples-in-paraffin). We deparaffinized the tissue slides according to the protocol and performed enzymatic antigen retrieval using 1:1 trypsin concentrates and buffer. Washes were done with 1×TBST. Blocking was done with protein block (ab64212) for 1 hr. After this, washes were repeated. Next, we incubated the slides with primary antibodies against MYC (anti-c-MYC 1:1000, cat no: 67447-1-lg) or with PD-L1 (E1L3N®) XP® rabbit mAb #13684) overnight. The next day, slides were washed with 1× TBST. The secondary antibody was added on the slide and incubated for 1 hr. Subsequently, we washed the slides with 1×TBST. To detect signal, we used the DAB and concentrated and enhanced with enhancer. We then counter stained the slides. The mounting media and cover slips were added, and the slides were sealed. Images were obtained on EVOS Fl at 40×. Target staining of the MYC and PD-L1 was quantified in Image J (https://imagej.net/ij/) RRID:SCR_003070.

Statistical Analysis: The drug dose response curve was performed in replicates and a minimum number of N=3. The in vivo experiments were performed with a minimum of 3 mice per group with dose dependency to validate therapeutic efficacy. Paired T-test was used to determine statistical significance between treated groups and controls. The Log Rank Mantel test was used to determine statistical significance between the in vivo treatment groups. The Kaplan Meier survival curve was used to determine survival differences between the various treatment groups and controls. All data was plotted with the GraphPad Prism (v.10) RRID:SCR_002798.

### Example 4B: Results

Dose dependent inhibition of the lethal pancreatic cancer by 3′UTRMYC1-18. Our aim was to validate the novel c-MYC mRNA destabilizing drug (3′UTRMYC1-18) the in vitro therapeutic efficacy of the metastatic drug-resistant pancreatic cancer cell lines MIA-PaCa-2 and PSN1 and PANC1. We performed a head-to-head dose dependent IC₅₀ determination with 3′UTRMYC1-18 and the standard of care drugs including a MYC-Max inhibitor (MYCi975). We found in that 3′UTRMYC1-18 achieved an IC₅₀ of 1.98 and 1.2 μM (FIG. 74A-C) which is superior to these standard of care drugs such as MYCi975 in PSN1 and MIA-Paca2 but not in the PANC1 cells. The MYC mRNA destabilizing drug engaged the MYC mRNA in a dose dependent manner (FIG. 74D). Notably, we found that the mRNA destabilizing drug synergizes when used in combination with the epirubicin (FIG. 74E).

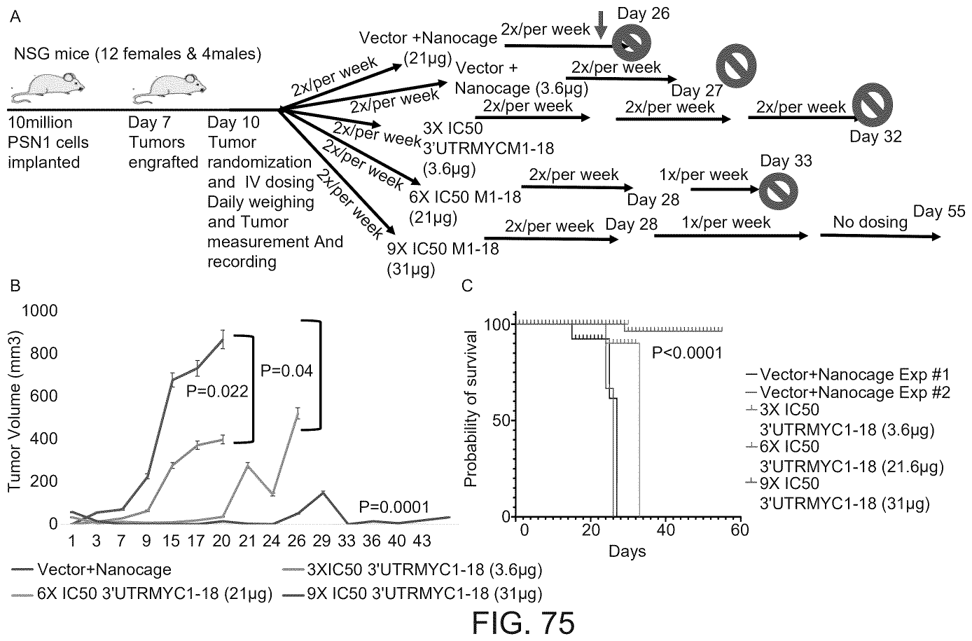

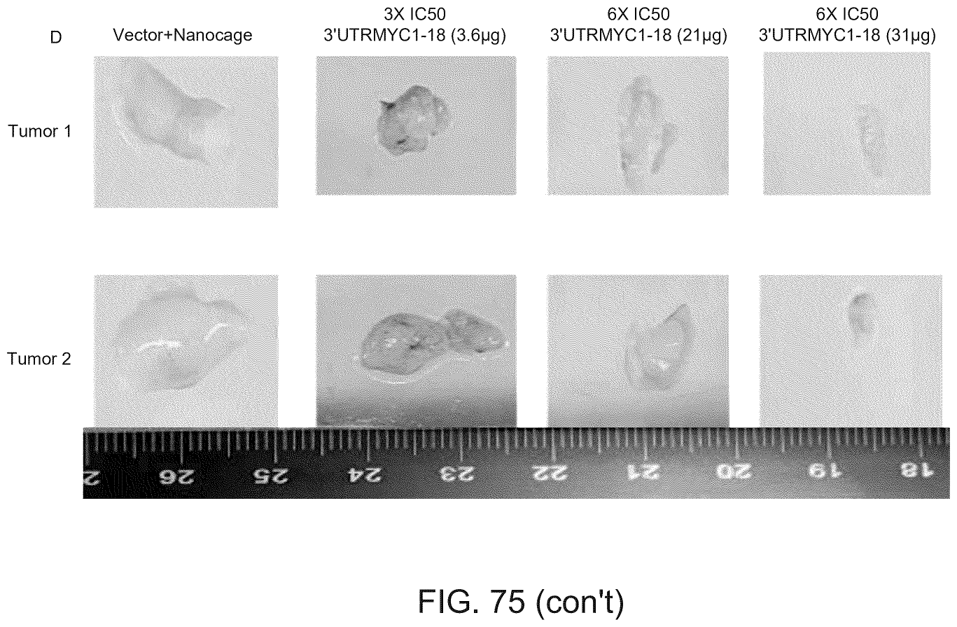

To prove the efficacy of the drug in vivo, two independent experiments were used for titrating the drug in dose dependent manner (FIG. 75A). In the first, we implanted 10 million PSN1 cells as xenograft into 5 females and 3 males NSG mice of the ages 5-8 weeks and weight of 15-21 g. After 7 days, the tumors engrafted, and on day 10, we randomized the animals into two groups with 4 mice per group according to tumor volume and weight. The groups were 1) vector+nanocage and 2) 3×IC₅₀ 3′UTRMYC1-18 (3.6 μg). We dosed the animals intravenously (IV-tail vein) 2×/week, and on day 25 (marked with inverted arrow) we collected blood for safety and toxicity profile analysis. On day 26, the vector+nanocage treated groups all died while the 3×IC₅₀ 3′UTRMYC1-18 treated groups lived until 32 days. The tumor bearing mice treated with c-MYC mRNA destabilizing drug (3.6 μg) show significant reduction of the tumor volume (*p=0.0119) compared to the controls (FIG. 75B-C), comparatively about half in tumor volume reduction (FIG. 75B). The animals treated with c-MYC mRNA destabilizing drug also survived longer than the vector+nanocage treated group (*p=0.0117; FIG. 75C).

Our next aim was to prove that c-MYC mRNA destabilizing drug inhibited c-MYC in vivo in a titratable dose dependent manner in lethal pancreatic cancer. We titrated the dosing of the MYC mRNA destabilizing drug from the first in vivo experiment of 3×IC₅₀ (3.6 μg) to 6×IC₅₀ (21.6 μg) and to 9×IC₅₀ (32 μg). The control was the vector+nanocage treated group (FIG. 75B). Briefly, we obtained 7 females and 1 male NSG mice and implanted 10 million PSN1 cells. After 1 week, the tumors were engrafted and we randomized the animals into 3 groups: 1) vector+nanocage, 2) 6×IC₅₀ 3′UTRMYC1-18 (21 μg) and 3) 9×IC₅₀ 3′UTRMYC1-18 (31 μg). The animals were dosed 2× per week intravenously (tail vein) till day 27-28 post implantation. On the day 27, the vector+nanocage treated groups died. The animals were then dosed 1× per week. On day 33, the 6×IC₅₀ treated group died. The 9×IC₅₀ treated group lived until 55 days post implantation. Our results showed that the 9×IC₅₀ group achieved complete in vivo inhibition of pancreatic cancer (FIG. 75B, FIG. 75D) with highly significant survival outcomes (****P<0.0001) (FIG. 75C).

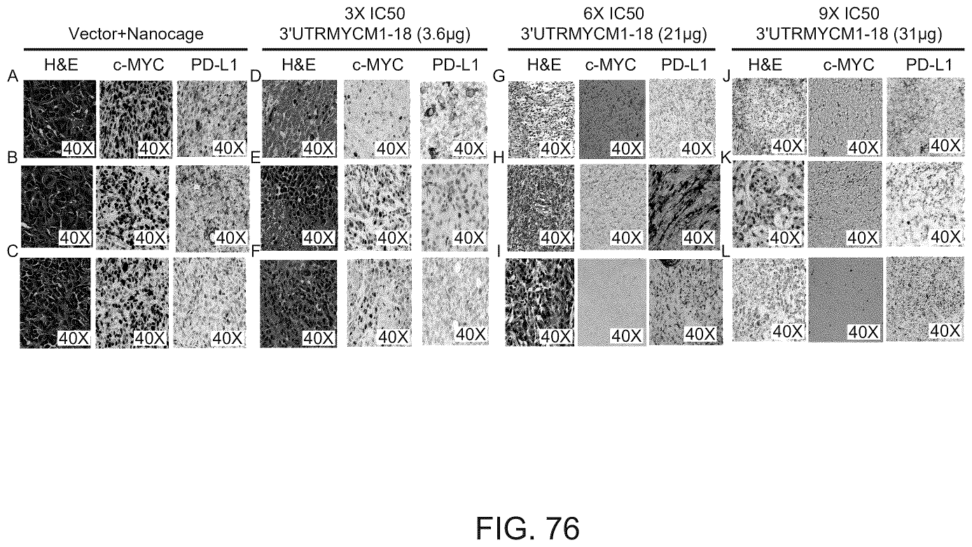

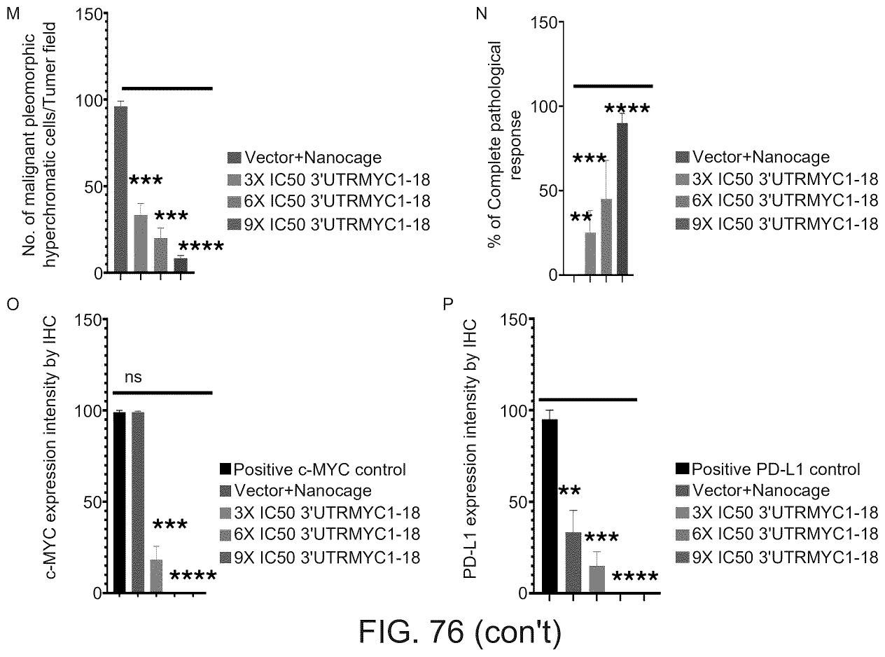

3′UTRMYC1-18 target inhibition of pancreatic tumors involves down regulation of the c-MYC and PD-L1. The next aim was to confirm that the MYC mRNA destabilizing drug achieved on target inhibition of c-MYC in the primary tumor to achieve therapeutic efficacy. We performed H&E staining of the tumors from the vector+nanocage treated group, and the 3×IC₅₀, 6×IC₅₀ and 9×IC₅₀ treated groups (3 tumors from each group). We found that the vector+nanocage treated tumors were hyperchromatic, malignant pleomorphic cells (FIG. 76A-C). 3×IC₅₀ treatment achieved complete pathological response in 1/3 tumors (one out of three) and partial response in 2/3 tumors (FIG. 76D-F). 6×IC₅₀ treatment achieved complete pathological response in 1/3 tumors, a partial response in 1/3 tumors, and no response in 1/3 of tumors (FIG. 76I). The 9×IC₅₀ dose achieved 100% complete response (3/3 tumors) (FIG. 76J-L). The results were quantified as shown in FIG. 76M-N.

Next, we stained for c-MYC expression by IHC in the 3 tumors from each group. We found a very high level of c-MYC in the vector+nanocage treated group (FIG. 76A-C), a moderate level of c-MYC expression in the 3×IC₅₀ dose treated tumor group (FIG. 76D-F) and complete inhibition of c-MYC expression in the 6×IC₅₀ and 9×IC₅₀ treated groups (FIG. 76G-I, J-L). The results were quantified as shown in FIG. 760. We found similar dose dependent down regulation for PD-L1 expression in the same tumors (FIG. 76A-L). The results were quantified as shown in FIG. 76P. Taken together, these data demonstrate that the c-MYC mRNA destabilizing drug achieved dose dependent on-target inhibition of the lethal pancreatic tumors and dose dependent down regulation of the c-MYC and PD-L1 expression.

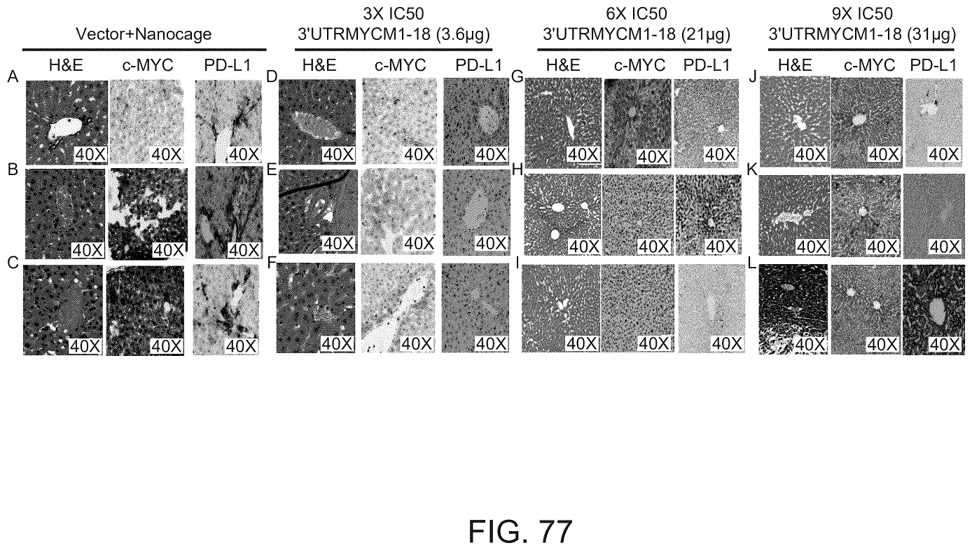

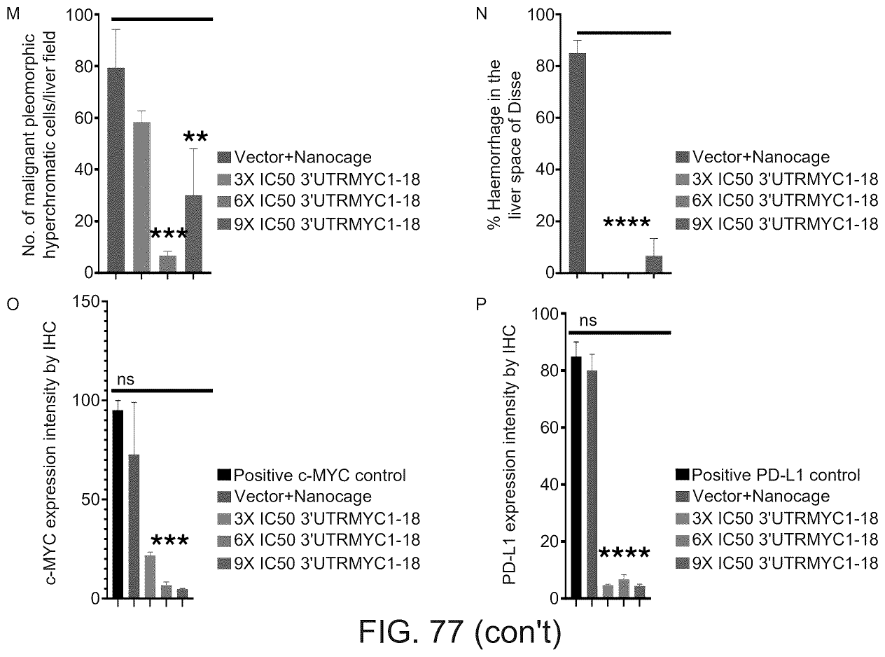

The down regulation of c-MYC inhibited liver, lung, and pancreatic cancer metastasis. The liver is the most common site of pancreatic cancer metastasis. We investigated whether the downregulation of the c-MYC and PD-L1 expression led to the inhibition of metastasis to the lungs and liver and pancreas. We performed H&E staining of 3 livers from each group: 1) vector+nanocage treatment, 2) the 3×IC₅₀ treatment, 3) 6×IC₅₀ treatment and 4) 9×IC₅₀ treatment of tumor bearing mice (FIG. 77). We found hyperchromatic pleomorphic cells (3/3), perforated (3/3) and eosinophilic hemorrhagic lesions (2/3) in the livers of vector+nanocage treated mice (FIG. 77A-C). 3×IC₅₀ 3′UTRMYC-18 treatment produced lower levels of hyperchromatic pleomorphic cells in each of the livers tested (3/3) fewer perforations in each of the livers tested (3/3) and no eosinophilic hemorrhagic lesions (0/3) in the livers of the tumor bearing mice treated (FIG. 77D-F). 6×IC₅₀ 3′UTRMYC1-18 treatment achieved more than 75% inhibition of the hyperchromatic pleomorphic cells (1/3), no perforations (0/3) and no eosinophilic hemorrhagic lesions (0/3) in the livers of the tumor bearing mice treated (FIG. 77G-I). 9×IC₅₀ 3′UTRMYC1-18 treatment achieved inhibition of the hyperchromatic pleomorphic cells (1/3), perforation (1/3) and hemorrhagic lesion (1/3) in the livers of the tumor bearing mice (FIG. 77J-L). The quantifications are shown in FIG. 77M-N.

We stained for the c-MYC and PD-L1 expression in the control and treatment groups. We found markedly elevated levels of c-MYC (2/3) and PD-L1 (3/3) in the livers treated with the vector+nanocage (FIG. 77A-C). We found moderate expression levels of c-MYC and PD-L1 in the 3×IC₅₀ dose treated group (FIG. 77D-F). Notably, we observed complete down regulation of c-MYC in the 6×IC₅₀ and 9×IC₅₀, treated groups (FIG. 77G-I, J-L). We found only one liver in the 6×IC₅₀ and 9×IC₅₀ treated groups with elevated PD-L1 expression (FIG. 77G-I, J-L). The quantifications are shown in FIG. 77O-P. Taken together, the results demonstrate dose dependent inhibition of liver metastasis and down regulation of c-MYC and PD-L1.

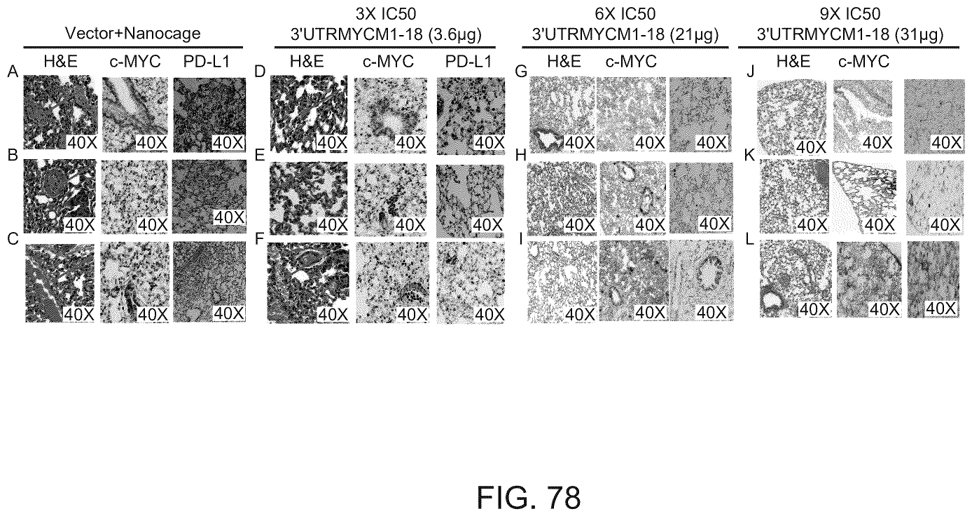

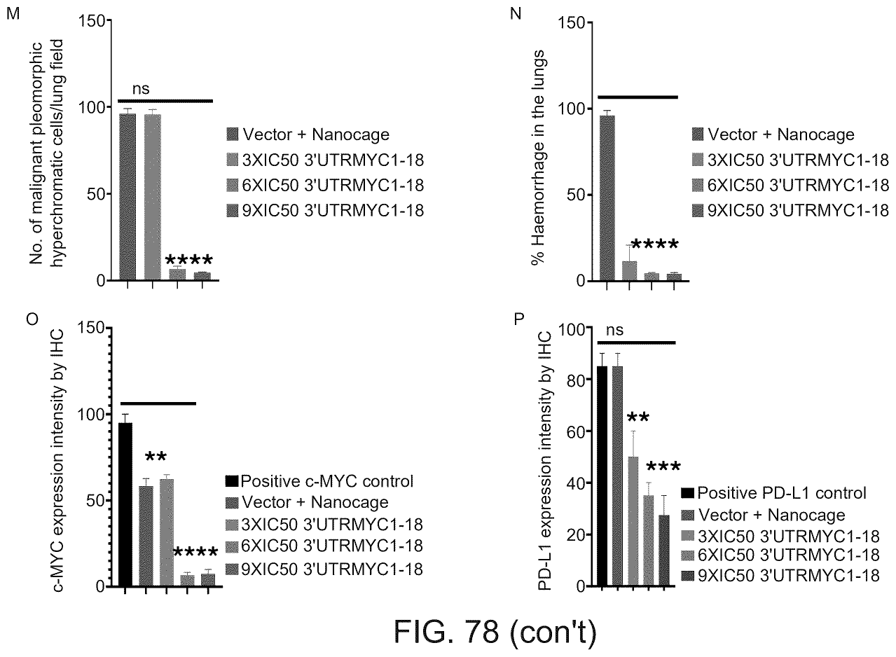

Next, we investigated whether 3′UTRMYC1-18 inhibited pancreatic cancer metastasis to the lungs. We performed H&E staining of the lungs of the tumor bearing mice treated with the vector+nanocage, the 3×IC₅₀ dose, the 6×IC₅₀ dose, and the 9×IC₅₀ dose. We found hyperchromatic, pleomorphic cells (3/3) and eosinophilic hemorrhagic lesions (3/3) in the lungs treated with the vector+nanocage (FIG. 78A-C). In the 3×IC₅₀ 3′UTRMYC1-18 dose treated mice, we found malignant hyperchromatic pleomorphic cells (3/3) and (1/3) eosinophilic hemorrhagic lesion in the lungs (FIG. 78D-F). In the 6×IC₅₀ and 9×IC₅₀ 3′UTRMYC1-18 dose treated lungs, we found complete inhibition of the malignant pleomorphic cells, and no eosinophilic hemorrhagic cell in the lungs (0/3) (FIG. 78G-I, J-L). These results were quantified as shown in FIG. 78M-N.

We extended our analysis into the c-MYC and PD-L1 expression down regulation, by comparing the expression of the c-MYC and PD-L1 in the lungs of the vector+nanocage (FIG. 78A-C) treated tumor bearing mice and the dose dependent treatment. We found a clear dose dependent downregulation of the c-MYC and PD-L1 in the lungs treated with 3×, 6× and 9×IC₅₀ 3′UTRMYC1-18 (FIG. 78D-F, G-I, J-K). The results were quantified as shown in FIG. 78O-P. Taken together, we demonstrate that the drug achieved distant organ lung metastasis inhibition by downregulating the c-MYC and PD-L1 in dose dependent manner.

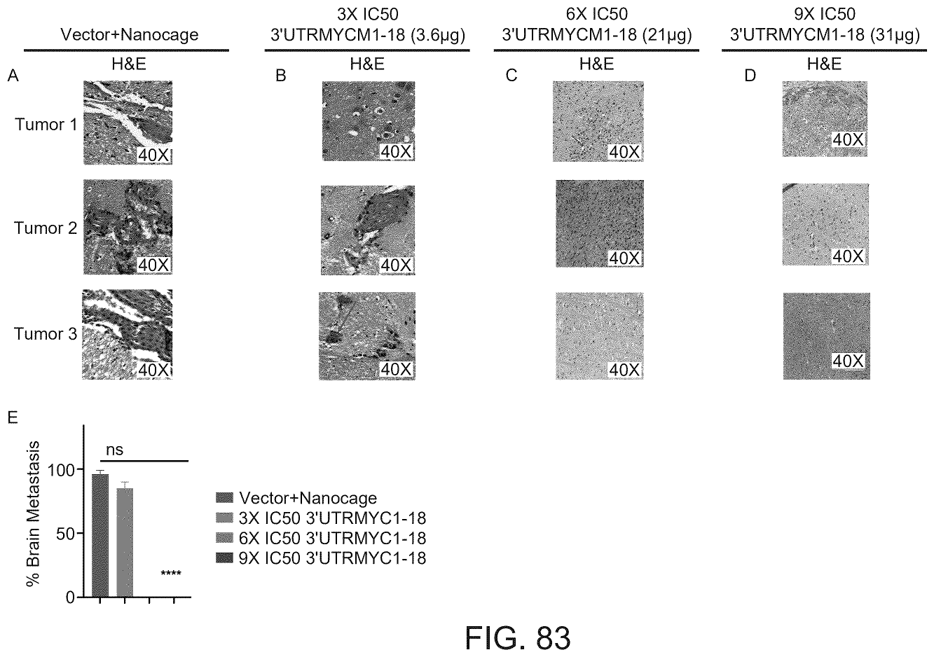

In the brain cells, we found an inhibition of brain metastasis in the pancreatic cancer tumor bearing mice treated with 6× and 9×IC₅₀ 3′UTRMYC1-18 (FIG. 83C-D) compared to the controls, vector+nanocage treatment, and 3×IC₅₀ 3′UTRMYC1-18 treatment (FIG. 83A-B). The inhibition of brain metastasis is dose dependent in the order 9×IC₅₀=6×IC₅₀>3×IC₅₀ 3′UTRMYC1-18>vector+nanocage. (FIG. 83A-D). The results were quantified as shown in FIG. 83E.

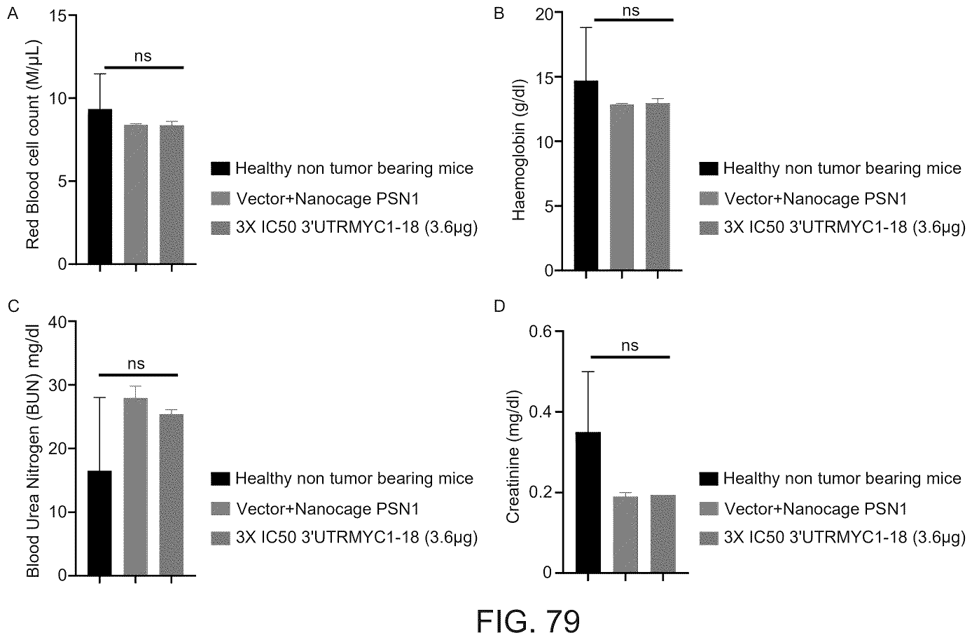

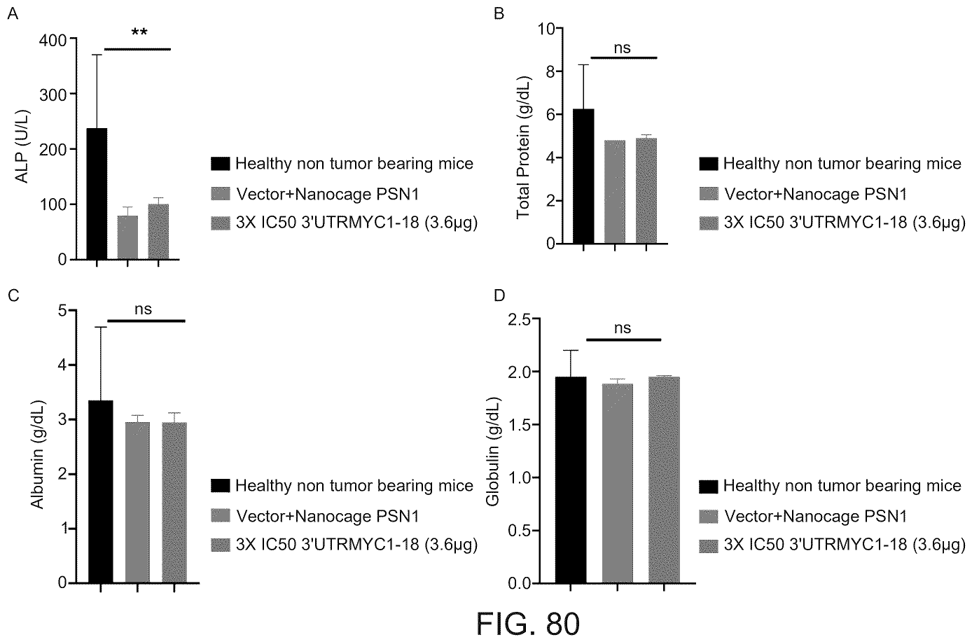

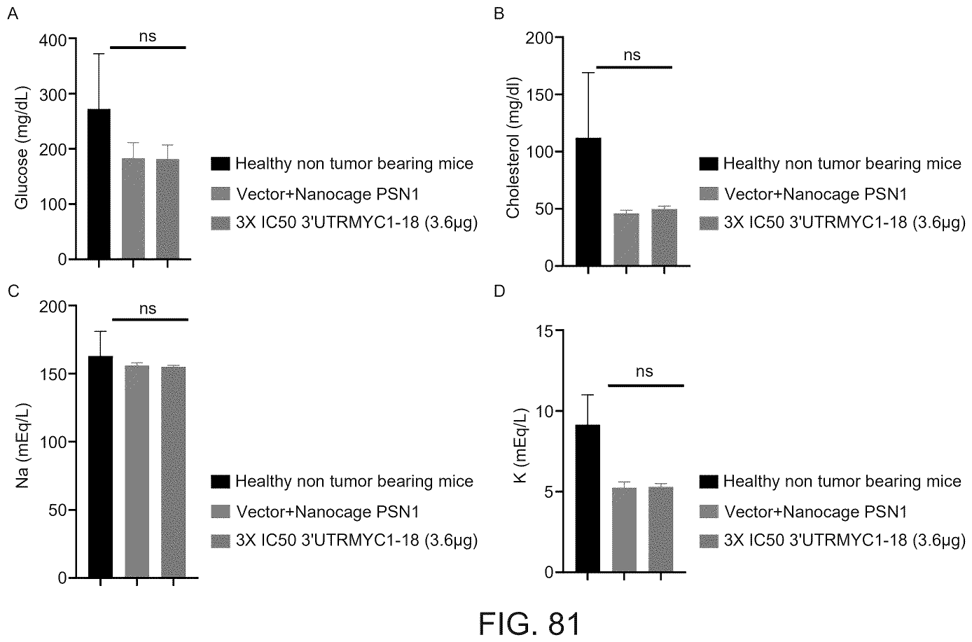

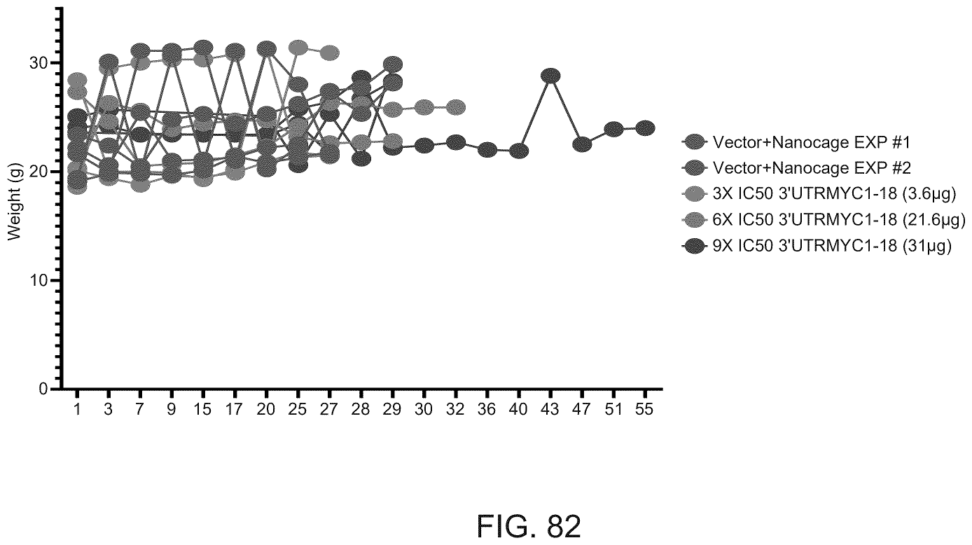

In vivo safety and toxicity profile analysis show that 3′UTRMYC1-18 is safe for administration. Our next aim was to prove drug safety. We collected the blood on day 25 (FIG. 74A) to assay blood cells, electrolyte levels, liver enzymes, kidney function, and pancreatic and biliary function (Table 4A and 4B). We found no change in red blood cells (FIG. 79A), hemoglobulin (FIG. 79B) and other blood indices (Table 4A). We found no change in the levels of the blood urea nitrogen (FIG. 79C) and no change in the weight of the animals that received the mRNA destabilizing drugs (FIG. 82). We found a normal range for creatinine levels (FIG. 79D). We examined the liver ALP levels (FIG. 80A) and other liver enzymes levels (Table 4B). We found that levels were reduced except for the AST levels which were elevated in one mouse treated with 3×IC₅₀ 3′UTRMYC1-18 (Table 4B). The total protein levels, albumin levels, and globulin levels were all within normal ranges (FIG. 80B-D). The glucose levels, cholesterol levels, and electrolyte (Na and K) levels were all within normal ranges (FIG. 81A-D and Table 4B). The results showed that the drug was safe and well tolerated with no blood dyscrasia, no electrolyte imbalance, no kidney failure, no liver failure, and no pancreatic enzyme abnormalities.

TABLE 4A
Complete blood count

| Accession | | | | |
|---|---|---|---|---|
| No .: | 24-6558-3 | 24-6558-4 | 24-6558-5 | 24-6558-6 |
| Sample ID: | 2A | 2B | 3A | 3B |
| Sex: | U | U | U | U |
| Test | 3X IC50 M1- | 3XIC50 M1- | V + N | V + N |
| sample: | 18 PSN1 | 18 PSN1 | PSN1 | PSN1 |

Red blood cell and platelet indices

| Marker | | | | | Reference |
|---|---|---|---|---|---|
| tested | | | | | range |
| RBC (M/uL) | 8.53 | 8.20 | 8.44 | 8.35 | 7.84-10.84 |
| HGB (g/dL) | 13.20 | 12.70 | 12.90 | 12.80 | 11.80-17.60 |
| HCT (%) | 43.00 | 41.70 | 42.40 | 41.90 | 44.10-58.30 |
| MCV (fL) | 50.40 | 50.90 | 50.20 | 50.20 | 51.10-58.60 |
| MCH (pg) | 15.50 | 15.50 | 15.30 | 15.30 | 13.70-17.20 |
| MCHC (g/dL) | 30.70 | 30.50 | 30.40 | 30.50 | 25.10-31.30 |
| RDW-SD (fL) | 26.70 | 27.20 | 27.30 | 26.40 | — |
| RDW-CV (%) | 16.00 | 16.00 | 16.40 | 16.20 | 17.30-20.30 |
| RET# (K/uL) | 530.60 | 406.70 | 419.50 | 499.30 | 294.00-444.00 |
| RET (%) | 6.22 | 4.96 | 4.97 | 5.98 | 2.56-4.56 |
| PLT (K/uL) | 1527.00 | 1408.00 | 871.00 | 1640.00 | 651.00-2055.00 |
| PDW (fL) | 6.40 | 6.50 | 6.90 | 6.00 | — |
| MPV (fL) | 6.60 | 6.70 | 7.00 | 6.60 | 4.20-6.30 |

Automated differentials

| Marker | | | | | Reference |
|---|---|---|---|---|---|
| tested | | | | | range |
| WBC# (K/uL) | 2.61 | 3.27 | 2.10 | 5.21 | 0.94-4.68 |
| NEUT# (K/uL) | 2.11 | 2.31 | 1.62 | 3.97 | 0.54-3.16 |
| LYMPH# (K/uL) | 0.12 | 0.29 | 0.24 | 0.59 | 0.23-1.56 |
| MONO# (K/uL) | 0.36 | 0.66 | 0.22 | 0.63 | 0.03-0.26 |
| EO# (K/uL) | 0.02 | 0.01 | 0.01 | 0.02 | 0.00-0.39 |
| BASO# (K/uL) | 0.00 | 0.00 | 0.01 | 0.00 | 0.00-0.15 |
| NEUT (%) | 80.80 | 70.60 | 77.10 | 76.20 | 44.21-79.92 |
| LYMPH (%) | 4.60 | 8.90 | 11.40 | 11.30 | 13.51-42.61 |
| MONO (%) | 13.80 | 20.20 | 10.50 | 12.10 | 1.71-10.93 |
| EO (%) | 0.80 | 0.30 | 0.50 | 0.40 | 0.29-10.32 |
| BASO (%) | 0.00 | 0.00 | 0.50 | 0.00 | 0.00-3.55 |

TABLE 4B
Chemistry panel

| | 2400655803 | 2400655804 | 2400655805 | 2400655806 | |
|---|---|---|---|---|---|
| | Sample ID: | | | | |
| | 2A | 2B | 3A | 3B | |
| | Sex: | | | | |
| | U | U | U | U | |
| | Test sample: | | | | |
| | 3X IC₅₀ M1- | 3XIC₅₀ M1- | V + N | V + N | Reference |
| | 18 PSN1 | 18 PSN1 | PSN1 | PSN1 | Range |
| BUN (mg/dL) | 29.8 | 26.1 | 24.7 | 26 | 5.00-28.00 |
| CREA (mg/dL) | 0.194 | 0.194 | 0.18 | 0.202 | 0.20-0.50 |
| BUN/CREA Ratio | 153.608 | 134.536 | 137.222 | 128.713 | — |
| ALP (U/L) | 112 | 89 | 64.5 | 94.9 | 105.00-370.00 |
| ALT (U/L) | 23.5 | 23.3 | — | 22.3 | 27.00-195.00 |
| AST (U/L) | 112.3 | 56.5 | 55.7 | 60.4 | 54.00-77.00 |
| GGT (U/L) | 0.1 | 0 | 0.3 | 0 | — |
| TBIL (mg/dL) | 0.122 | 0.114 | 0.104 | 0.12 | 0.20-0.60 |
| DBIL (mg/dL) | 0.022 | 0.022 | — | 0.017 | — |
| IBIL (mg/dL) | 0.100 | 0.092 | — | 0.103 | — |
| TP (g/dL) | 5.01 | 4.78 | 4.8 | 4.88 | 4.80-7.20 |
| ALB (g/dL) | 3.07 | 2.82 | 2.87 | 3.04 | 2.40-4.30 |
| GLOB (g/dL) | 1.940 | 1.960 | 1.930 | 1.840 | 1.70-2.20 |
| A/G Ratio | 1.616 | 1.410 | 1.5 | 1.689 | — |
| P (mg/dL) | 7.94 | 8.93 | 7.49 | 8.74 | 7.30-14.50 |
| Ca (mg/dL) | 9.55 | 9.67 | 9.76 | 9.88 | 9.50-12.50 |
| GLU (mg/dL) | 156.4 | 207.6 | 155 | 211.7 | 172.00-372.00 |
| CHOL (mg/dL) | 47.1 | 52.3 | 43.4 | 48.7 | 55.00-169.00 |
| TRIG (mg/dL) | 112.3 | 120.3 | 70.5 | 74.5 | 67.00-289.00 |
| CK (U/L) | 33.8 | 46.7 | — | 2.6 | 428.00-1609.00 |
| TCO2 (mEq/L) | — | 3.5 | — | 7.7 | — |
| Na (mEq/L) | 156 | 154 | 158 | 154 | 145.00-181.00 |
| K (mEq/L) | 5.5 | 5.1 | 5.6 | 4.9 | 7.30-11.10 |
| CL (mEq/L) | 116 | 116 | 119 | 113 | 111.00-134.00 |
| Na/K | 28 | 30 | 28 | 31 | — |
| Anion Gap | — | 40 | — | 38 | — |

Taken together, we present very strong evidence that the MYC mRNA destabilizing drug is effective as monotherapy in in vitro and in vivo in pancreatic cancer models. This data provides evidence that the MYC mRNA destabilizing drug will be an effective therapy for patients suffering from pancreatic cancer.

### Example 4C: Discussion

Pancreatic ductal carcinoma is the most common form of pancreatic cancer and is the deadliest with a survival rate of 11% (1c-2d). Pancreatic cancer is very aggressive and ultimately lethal, metastasizing to the duodenum, liver, mediastinum, lungs, pericardium, and the brain. The drugs used for pancreatic cancer are gemcitabine, capecitabine or FOLFIRINOX (3d-6d). Chemoresistance starts 21 months after this chemotherapy due to the upregulation of the c-MYC oncogenic signal. About 43.1-74.7% of the primary and metastatic drug-resistant pancreatic cancers express MYC (7c-9c). This is a significant challenge, and it illustrates a critical need for improved clinical outcomes.

c-MYC is a basic helix loop helix transcription factor that binds the E-box sequences. This group of transcription factors is a super family which comprises of the c-MYC, MYCN and the MYC (10c-11c). MYC is over expressed in a variety of human cancers through amplification, insertion, and rearrangements. To date there is no direct MYC inhibitor that is clinically available. To directly target MYC, we developed a destabilized c-MYC 3′UTR mRNA destabilizing drug based on engineering the mRNA poly U stabilizing elements. The drug works by directly and specifically recognizing the endogenous c-MYC mRNA exons. When ribosomes attempt translation in the presence of the drug, the destabilized elements are recognized. This in turn triggers the EXOSC4-PELO-RPL3 complex to degrade the destabilized MYC mRNA leading to the downregulation of the MYC protein (12c-13c, 14c-15c).

We have demonstrated in this study that the lethal pancreatic cancer driven by c-MYC can be inhibited with the c-MYC mRNA destabilizing drug in a dose dependent titratable manner to achieve significant survival outcomes. The 3×IC₅₀ and 6×IC₅₀ doses achieved a 6-day survival difference compared to the control. Given that a day of mice life is equivalent to 40 days of human life, this equates to a 240-day survival benefit for the 3× and 6×IC₅₀ 3′UTRMYC1-8 doses respectively. When we increased the dose to 9×IC₅₀, we achieved a 28-day survival difference compared to the control. This equates to a 3 yr and 1 month survival difference for human patients.

The disclosed mRNA destabilizing drug is clearly superior to the available drugs used for pancreatic cancer. In particular, gemcitabine, capecitabine and FOLFIRINOX achieved a survival outcome of only 21.4 months in human equivalents. Therefore, 3′UTRMYC1-18 is highly effective in treating pancreatic cancer in vivo and at the same time 3′UTRMYC1-18 shows a very positive safety profile. As such, 3′UTRMYC1-18 is useful for the treatment of diverse c-MYC driven cancers including lethal pancreatic cancers of all stages.

In conclusion, we have demonstrated the generalizability of the MYC mRNA destabilizing drug therapeutic efficacy across different pancreatic cancer models of PSN1, MIA-Paca-2 and PANC1. We have validated the inhibition of c-MYC expression in PSN1 in vivo with highly favorable survival outcomes. The model used herein has been an NSG metastatic xenograft model. The testing will be extended to genetically modified mouse model (GEMM) of pancreatic cancer. This will give further insight into how the immune cells and check point proteins inhibition support the therapeutic efficacy of the drug.

### Example 5: In Vivo Inhibition of c-MYC in Metastatic Drug-Resistant Ovarian Cancer Cells and Downregulation of c-MYC-PD-L1-PAX8-p21 to Achieve Therapeutic Efficacy

Metastatic drug-resistant ovarian cancer is the deadliest form of gynecological cancer. It afflicts women globally with over 49% relapsing after initial diagnosis and surgery and treatment. High-grade serous tumors (HGSOC) are most commonly diagnosed. In the USA, 21,000 patients are diagnosed annually with more than half dying from the disease due to metastasis and drug resistance. The mainstay therapy for ovarian cancers is platinum-based therapies such as cisplatin and similar drugs. Yet, drug resistance is very likely due to pervasive oncogenic signals driving this resistance. One such oncogenic signal is from c-MYC. About 30-60% of high grade serous and drug resistant (paclitaxel and carboplatin) ovarian cancers overexpress c-MYC. This c-MYC overexpression is a major driver of chemoresistance leading to progressive disease and death of the patients. Here, we show that the c-MYC mRNA drug, 3′UTRMYC1-18, achieved dose dependent and titratable down regulation of c-MYC mRNA The IC₅₀ for 3′UTRMYC1-18 was found to be superior to the standard of care drugs with anti-cancer migration and viability properties. In vivo experiments involving patient-derived ovarian cancer PDX show that c-MYC mRNA drug achieved very significant treatment outcomes through the down regulation of the c-MYC-PDL-1-PAX8 and p21. We thereby provide a novel therapeutic method for targeting drug resistant ovarian cancer cells.

### Example 5A: Materials and Methods

Cell culture: We obtained the following ovarian cancer cells for experimentation. A2780 and OVCAR8 cells were obtained from the Sigma Aldrich. The primary ovarian cancer cells were patient derived xenograft cells OCI-9x, P5X and C5X were obtained from Dr Tan Ince, Chief Pathologist of the Weill Cornell New York Presbyterian Hospital as a kind gift (Tan Ince, Nat Comm). The A2780 and OVCAR8 cells were grown in RPMI media supplemented with 10% FBS and antibiotics/antimycotics. The OCI-9×, P5X and C5X cells were grown in the primary ovarian cancer maintenance media (US Biologicals. Cat no: 506390). The cells were grown to 80% confluency before use.

Dose dependent IC₅₀ determination and comparison with standard of care drugs: We performed dose dependent titration of 3′UTRMYC1-18 in head-to-head comparison with the standard of care drugs. These included olaparib, paclitaxel, cisplatin, epirubicin, bevacizumab, and MYCi975. All drugs were obtained from Selleckchem (USA). We prepared serial dilutions for the drugs starting from 2.5 μg to 40 μg. We seeded the cells at 5,000 cells per well in a 96 well plate. The cells were allowed to be attached for 24 hrs and then the drugs were added. We incubated the treated cells for 72 hrs. Using CELLTITER-GLO (Promega G7570), viability was read and normalized to the controls. The data curves were fitted on a drug dose response chart in GraphPad Prism (USA). From this, the IC₅₀ values were derived.

Quantitative reverse transcript PCR: We extracted the RNA using the Qiagen RNeasy kit (Cat No. 74104). The RNA was stored at −80° C. before use. To reverse transcribe the RNA, we used the Superscript IV reverse transcriptase kit (Cat No. 18090200) to make cDNA. We designed the qPCR primers targeting the exons of the c-MYC exon and GAPDH as housekeeping gene control. All the primers used are already published in (12d) The delta CT was used to normalize the transcript expression.

Migration assay: Briefly, 5,000 A2780 cells were seeded in 6 well plates and allowed to attach and reach 80% confluency. Subsequently, equal wound scratches were made. The distance of the wound closure was measured every 24 hrs for 4 days. The data was plotted in GraphPad Prism (RRID:SCR_002798).

Iron oxide nanocage and 3′UTRMYCN mRNA drug complexation. The iron oxide nanocage and the MYCN mRNA drugs were complexed in a 1:1 ratio (12d, 18d). The conjugates were incubated over night before use at 4° C. The drug conjugate was found to be stable at room temperature, 4° C., and −20° C. until use.

Animal Study: We sought to demonstrate in vivo therapeutic efficacy of the c-MYC mRNA destabilizing drug in metastatic primary ovarian cancer. We obtained IACUC institutional approval from the CUNY institutional review board. We ordered 8 female NSG mice, aged 5-8 weeks, with weights of 15-21 g (Jackson laboratory). Once received, we allowed the mice to acclimatize according to the protocol. We implanted 10 million P5X cells orthotopically into the mice. After 35 days, the tumors were engrafted in the ovaries. On day 36, we randomized the animals according tumor size and weight into two groups: 1) vector+nanocage treatment, 2) IC5₀ 3′UTRMYC1-18 treatment (10 μg). We started intravenous (tail vein) administration of the controls and the drug, 2× per week until day 77. After this point, the vector+nanocage group exceeded the tumor volume and were euthanized. The IC₅₀ 3′UTRMYC1-18 group were not dosed after day 77 days until the end of the experiment on day 86. We recorded daily tumor volumes, weight, and body condition scores. The animals were euthanized with CO₂ at the end of the study. The log rank Mantel test was used to determine statistical significance between the treatment groups. The Kaplan Meier survival curve was used to determine survival differences between the various treatment groups and controls. Animals were weighed and body condition scored daily. Tumor volumes were measured with calipers as ½XLXWXW and recorded and documented.

Necropsy: Upon death, the fresh carcasses were dissected. We collected the tumors, ovaries, lungs, kidneys, livers, and brain. We rinsed the lungs in 1×PBS and took images of the fresh organs. Subsequently, we collected fresh tissues of tumors and organs and froze in −80° C. All tissues were then placed in 4% paraformaldehyde until sent to the pathologist lab for embedding and H&E staining.

H&E staining of tumors and organs: QC embedded and sectioned the tissues according to standard protocol. The JPD performed the H&E staining. Images were obtained on the EVOS FL microscope at 40× magnification. Quantification was done in Image J (RRID:SCR_003070)(https://imagej.net/ij/) and in Graph Pad Prism (v10) RRID:SCR_002798.

TIC staining of c-MYC, PD-L1, PAX8, p21 of tumors and organs: Briefly, JPD performed IHC staining against c-MYC, PD-L1, PAX8, and p21 on the tumors and organs of the treated animals and the controls. We used the Abcam IHC protocol (https://www.abcam.com/en-us/technical-resources/protocols/ihc-with-samples-in-paraffin). We deparaffinized the tissue slides according to the protocol and performed enzymatic antigen retrieval using 1:1 trypsin concentrates and buffer. Washes were done with 1×TBST. Blocking was done with protein block (ab64212) for 1 hr. After this, washes were performed again. Next, we incubated the slides with primary antibodies overnight. The next day, slides were washed with 1×TBST. The secondary antibody was added on the slide and incubated for 1 hr. Subsequently, we washed the slides with 1×TBST. To detect the signals, we used the DAB and concentrate and enhanced with enhancer. After this, we counter stained and mounting media was added. The cover slip was placed on and sealed. Images were obtained on EVOS Fl at 40×. Target staining was quantified in Image J (https://imagej.net/ij/) RRID:SCR_003070.

Statistical analysis: All experimental data was performed in replicates and a minimum number of N=3. Paired T-test was used to determine statistical significance between treated groups and controls. The log rank Mantel test was used to determine statistical significance between the in vivo treatment groups. The Kaplan Meier survival curve was used to determine survival differences between the various treatment groups and controls. All data was plotted with the GraphPad Prism (v.10) RRID:SCR_002798.

### Example 5C: Results

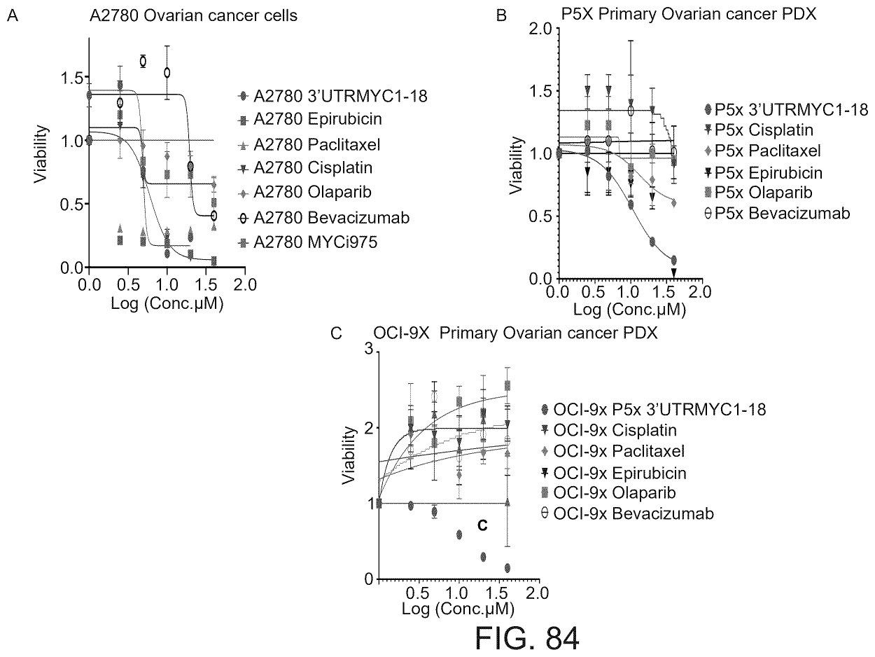

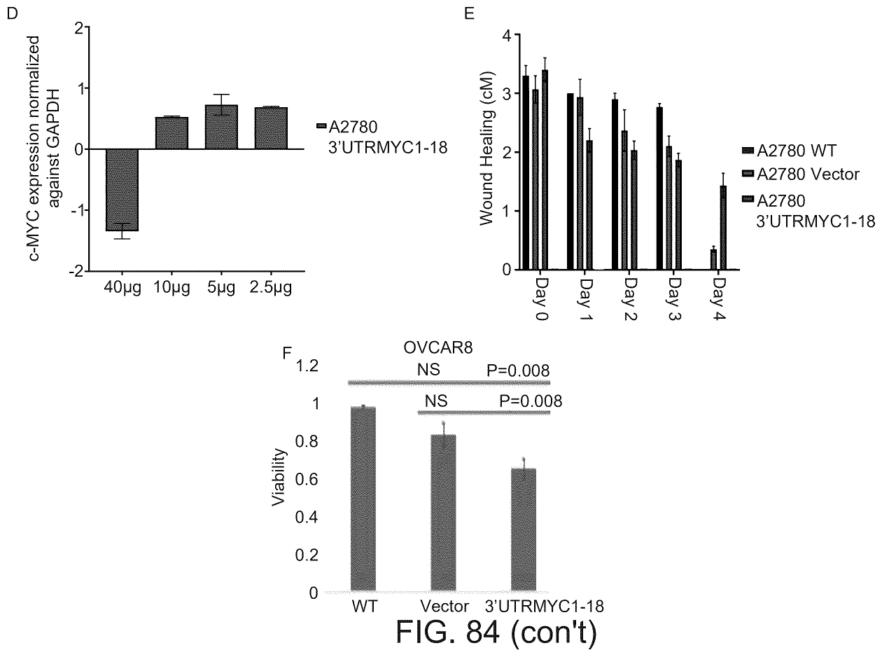

3′UTRMYC1-18 achieved a dose dependent titratable inhibition of drug-resistant ovarian cancer cell lines and primary ovarian cancer, PDX. We created 3′UTR mRNA destabilizing drug, 3′UTRMYC1-18, as described herein. As set out in this disclosure, we have used this drug to destabilize and degrade oncogenic c-MYC across different cancers and achieved complete pathological response in vivo with significant survival outcomes. In the above noted experiments, 3′UTRMYC1-18 was tested in metastatic drug-resistant ovarian cancer cell lines and patient derived xenografts. We performed head-to-head dose dependent IC₅₀ determinations with 3′UTRMYC1-18 and standard drugs including paclitaxel, olaparib, epirubicin, cisplatin, bevacizumab, and MYCi975. We found in that 3′UTRMYC1-18 achieved an IC₅₀ of 4.4 μM in A2780 (FIG. 84A; Table 3A, above). This is superior to cisplatin at 7.8 μM, olaparib at >40 μM, bevacizumab at 18.22 μM, and MYCi975 at 20 μM. In particular, 3′UTRMYC1-18 achieved an IC₅₀ of 10 μM higher than the IC₅₀ of these standard drugs (FIG. 84B-C). 3′UTRMYC1-18 engaged MYC mRNA in a dose dependent manner (FIG. 84D) and degraded c-MYC mRNA in a dose dependent manner. 3′UTRMYC1-18 inhibited the ovarian cancer migration compared to the controls (FIG. 84E) and impaired the cancer cell viability (FIG. 84F).

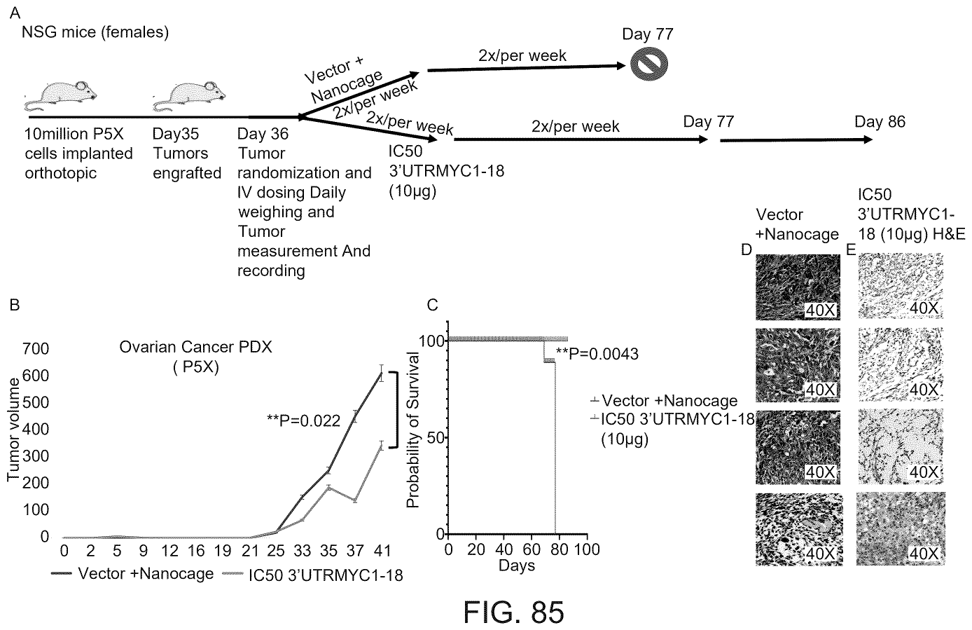

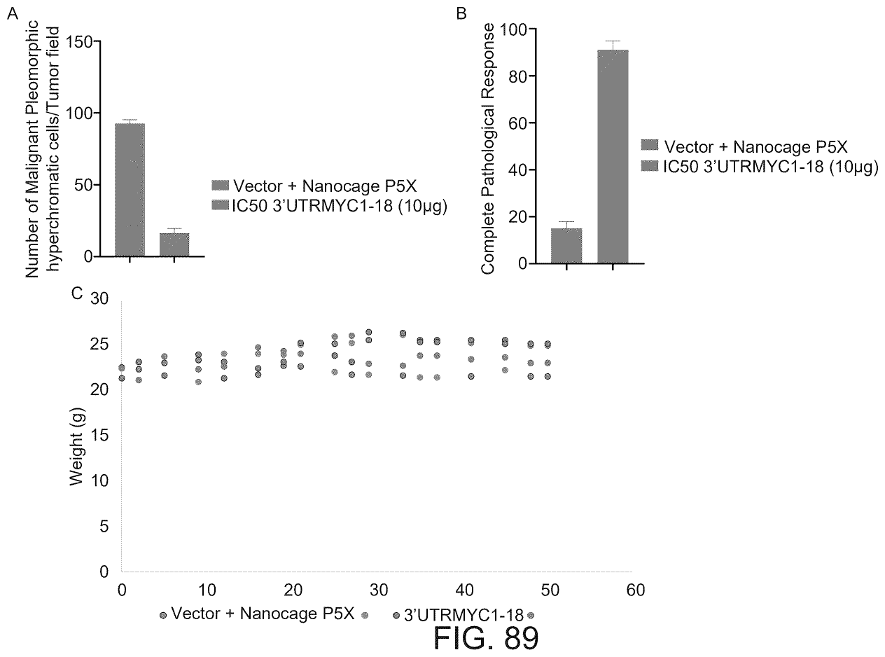

The MYC mRNA destabilizing drug inhibited ovarian cancer in vivo with significant survival outcomes. We next sought to validate in vivo therapeutic efficacy of 3′UTRMYC1-18 in PDX. To initiate these experiments, we obtained institutional review board IACUC approval. We implanted the ovarian cancer PDX cells, P5X (3d). 10 million cells were implanted orthotopically into female NSG mice having weights of 15-20 g and ages of 5-8 weeks. After 35 days, the tumors were engrafted. We then randomized animals into two groups: 1) vector+nanocage treatment, and 2) IC₅₀ 3′UTRMYC1-18 treatment (FIG. 85A). We treated the tumor bearing animals 2×/per week with daily tumor volume recording (½XLXWXW) weight measurement (FIG. 89C) and body condition score recording. On day 77, the vector+nanocage treated animals had exceeded tumor volume limit and were euthanized. 3′UTRMYC1-18 treated mice were alive up to 86 days (FIG. 85A).

At 41 days post-implantation, tumor volume measurements showed a clear difference between the vector+nanocage treated group and the 3′UTRMYC1-18 treated group. The 3′UTRMYC1-18 treated group had a significant reduction of tumor volume (**p=0.022) compared to the control (FIG. 85B). The 3′UTRMYC1-18 treated group show a very significant improvement in survival outcome (**p=0.0043) compared to the controls (FIG. 85C). Taken together, the data demonstrated the in vivo therapeutic efficacy of the c-MYC mRNA destabilizing drug in metastatic drug-resistant ovarian cancer cells derived from a patient xenograft.

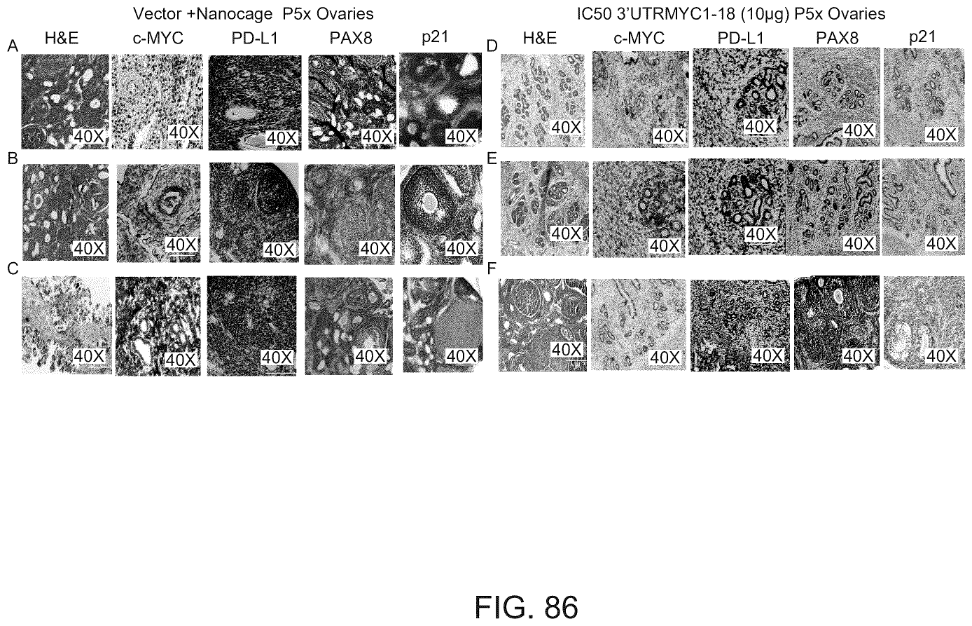

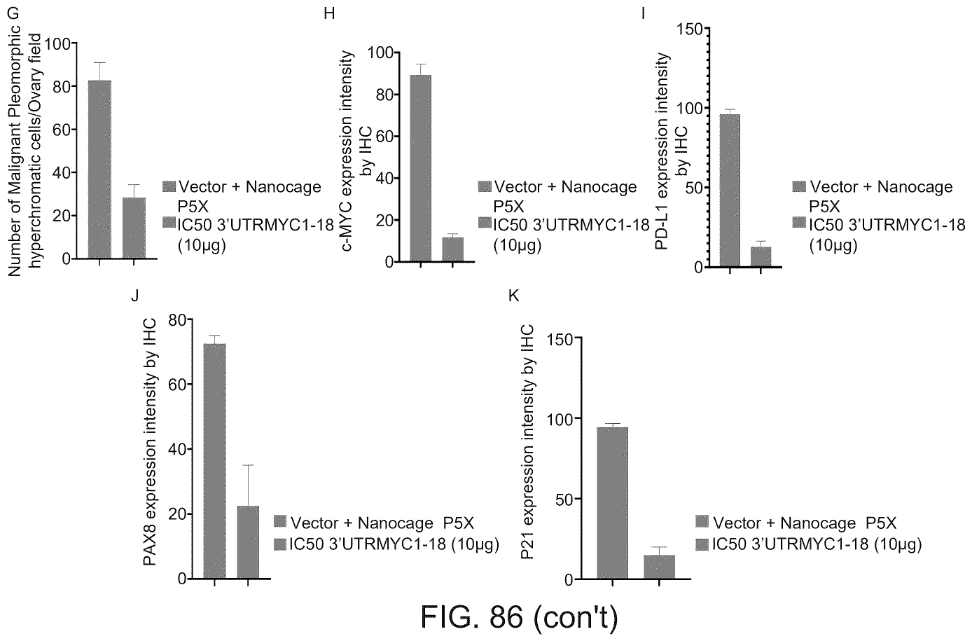

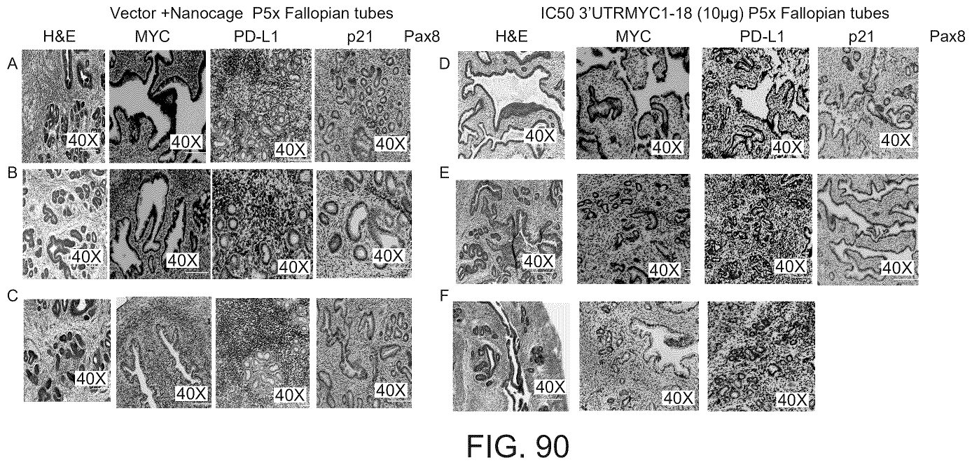

The c-MYC mRNA destabilizing drug achieved on-target inhibition c-MYC-PD-L1-PAX8-P21 to inhibit the primary tumors, tumors in the ovaries and the fallopian tubes. To demonstrate inhibition of c-MYC in the tumors, we performed H&E staining for the primary ovarian cancer PDX cells (FIG. 85D-E). We found that 3′UTRMYC1-18 treatment inhibited the malignant pleomorphic ovarian cancer cells compared to the controls (FIG. 85D-E). We found a nearly complete pathological response from 3′UTRMYC1-18 treatment (FIG. 89A-B). To show that 3′UTRMYC1-18 inhibited and reduced metastasis to the ovaries, we performed H&E and IHC staining of the ovaries with antibodies against the c-MYC, PD-L1, PAX8 and P21. We found a very high level of the c-MYC in the ovaries of the tumor bearing mice treated with the vector+nanocage (FIG. 86A-C, G). In the ovaries of the tumor bearing mice treated with the IC₅₀ dose (10 μg) of 3′UTRMYC1-18, we found greater than 90% reduction of the c-MYC expression compared to the controls (FIG. 86D-F, H). This was quantified as shown in FIG. 86H. PD-L1 expression is known to be markedly elevated in ovarian tumors. We found that PD-L1 expression was markedly elevated in the ovaries treated with the vector+nanocage (FIG. 86A-C, I). However, in the ovaries of 3′UTRMYC1-18 treated mice, PD-L1 expression was reduced by 80% (FIG. 86D-F, I). This was quantified as shown in FIG. 86I. The PAX8 protein has been implicated in the proliferation and viability, migration, and invasion of ovarian cancers. It has also been implicated as regulator of mutant p53, which promotes p21 action in anti-apoptotic and proliferative functions. We stained the ovaries of the vector+nanocage treated and 3′UTRMYC1-18 treated animals for PAX8 and p21 expression using IHC. We found very high levels of PAX8 and p21 in the ovaries of vector+nanocage treated mice (FIG. 86A-C, J-K). Contrasted to this, PAX8 and p21 expression was reduced about 70% in the ovaries of 3′UTRMYC1-18 treated mice (FIG. 86D-F, J-K). These levels were quantified as shown in FIG. 86J-K. In the fallopian tubes, we did not notable changes between vector+nanocage treatment and 3′UTRMYC1-18 treatment as relating to H&E, c-MYC, PD-L1, PAX8 and p21 staining (FIG. 90A-F). Taken together, we have demonstrated the therapeutic efficacy of 3′UTRMYC1-18 in vivo in inhibiting c-MYC-PD-L1-PAX8-p21 expression and in inhibiting metastasis in the ovaries.

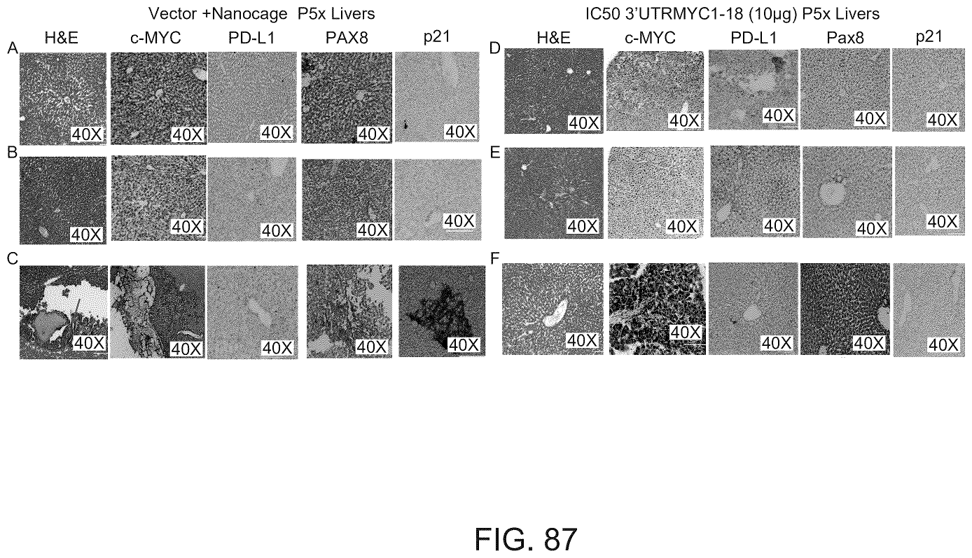

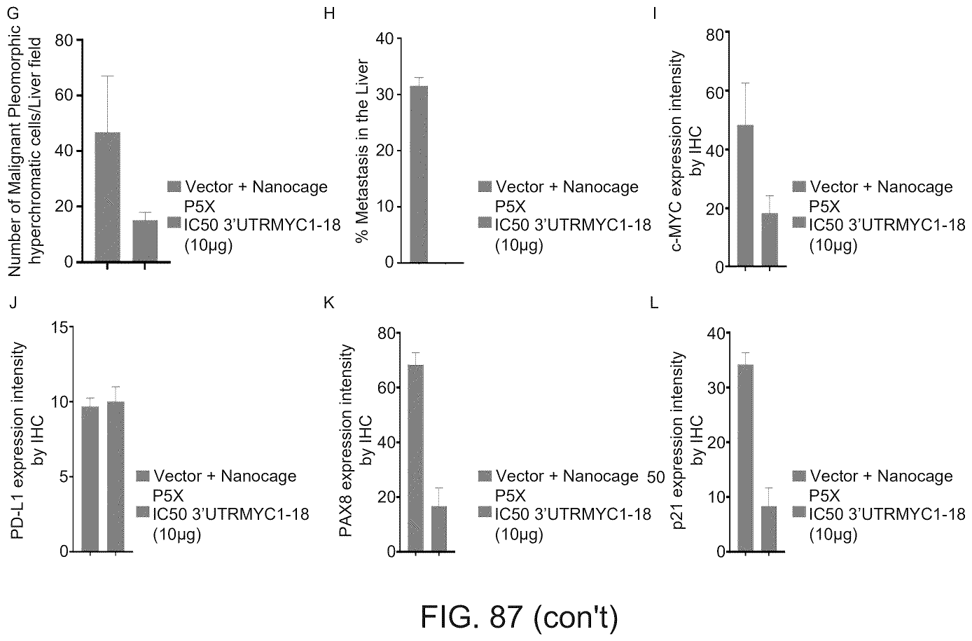

The c-MYC mRNA destabilizing drug inhibits liver and lung metastasis in aggressive metastatic ovarian cancer. The liver is the most common site of ovarian cancer metastasis. We stained the liver tissues of the control and 3′UTRMYC1-18 treated tumor bearing animals. We found metastasis (1/3) and malignant pleomorphic hyperchromatic cells (2/3) in the livers from the vector+nanocage treated group (FIG. 87A-C). We found no metastasis or malignant cells in the liver tissues (3/3) from the 3′UTRMYC1-18 treated group (FIG. 87D-F). c-MYC expression was found to be very high in the livers (3/3) of the tumor bearing mice treated with the vector+nanocage (FIG. 87A-C). In comparison to this, c-MYC expression was found to be low (2/3) of the tumor bearing mice treated with 3′UTRMYC1-18 (FIG. 87D-F).

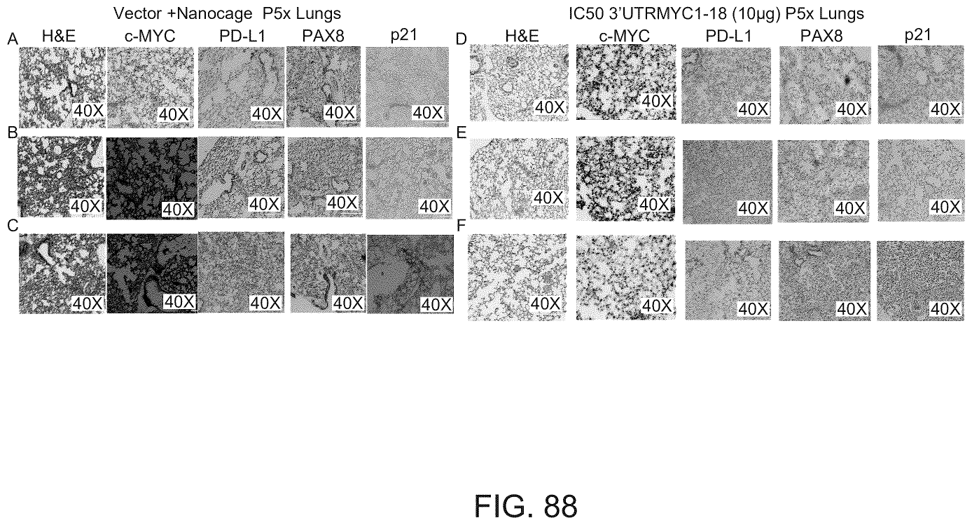

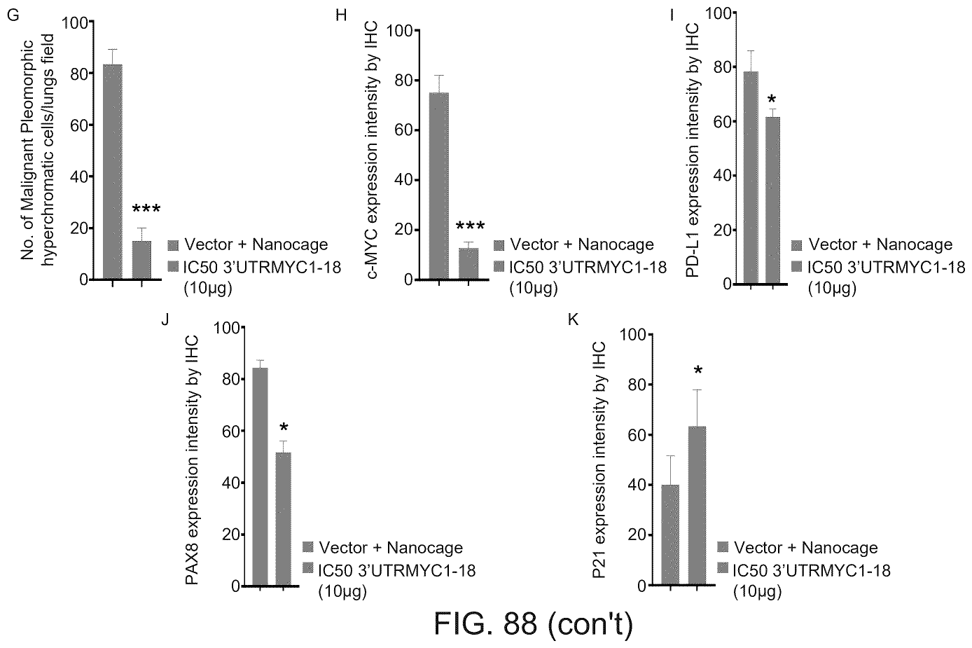

The lungs are also a common site for the ovarian cancer metastasis. We examined the lungs by H&E staining. We found that the lungs from animals treated with vector+nanocage show increased number of malignant hyperchromatic pleomorphic cells and loss of the lung architecture (FIG. 88A-C). In contrast, 3′UTRMYC1-18 treated animals show normal lung architecture and very significant reduction in the malignant hyperchromatic pleomorphic cells in the lung's parenchyma (FIG. 88D-F).

We stained for the c-MYC and observed down regulation of c-MYC in the 3′UTRMYC1-18 treated tumor bearing animals as compared to the controls (FIG. 88D-F, J). There was also some down regulation of the PD-L1 and the PAX8 in the 3′UTRMYC1-18 treated tumor bearing mice as compared to the controls (FIG. 88D-F, I). p21 was slightly elevated in the lungs of the 3′UTRMYC1-18 treated animals as compared to the controls. The data were quantified as shown in FIG. 88G-L. In conclusion, we found that the c-MYC mRNA destabilizing drug inhibits metastasis of ovarian cancer to the liver and to the lung and thereby provides significant survival outcomes.

### Example 5D: Discussion

We have demonstrated that metastatic drug-resistant c-MYC driven ovarian cancer can be treated by a c-MYC 3′UTR mRNA destabilizing drug. We show therapeutic efficacy both in vitro and in vivo with a very significant survival outcomes in tumor bearing mice treated with 3′UTRMYC1-18. The drug achieved on target inhibition of the c-MYC both on the mRNA level and the protein level. In vivo, the drug destabilized and inhibited c-MYC in the ovaries, livers, and lungs. We found that the inhibition of the c-MYC led to the downregulation of the PAX8, PD-L1 and p21 in the target organ, the ovaries.

The PAX8 is a master transcription factor implicated in ovarian cancer and other cancers. It belongs to the bHLH family. It is plausible that the c-MYC downregulated the PAX8 based on the shared binding sites or direct interactions as both are bHLH binding proteins. It has been shown by others that the PAX8 regulates the c-MYC in p53 mutant uterine cancers and that drugging PAX8 is an indirect way of targeting the c-MYC. Akin to this is the down regulation of the p21 which is involved in cell cycling. The inhibition of c-MYC, a pro cell cycle proliferative molecule can be used for down regulation of p21, and vice versa.

PD-L1 is over expressed in high grade serous ovarian cancer. It is a maker for poor prognosis but various PD-L1 inhibitors have failed in clinical trials for the treatment of ovarian cancers. c-MYC is a direct regulator of the PD-L1 through its binding site on the CD274 gene. We show here in ovarian cancer as well as other cancers that 3′UTRMYC1-18 inhibition of c-MYC concomitantly down regulates PD-L1. This indicates that 3′UTRMYC1-18 would be effective in targeting c-MYC and PD-L1 positive ovarian cancers. We show that 3′UTRMYC1-18 treatment inhibits liver and lung metastasis from the metastatic ovarian cancer cells. Moreover, the inhibition of the primary ovarian tumor and metastasis to the lungs and liver leads to significant survival outcome of the animals treated with 3′UTRMYC1-18. The findings demonstrate that 3′UTRMYC1-18 can indeed down regulate c-MYC in metastatic ovarian cancer and inhibit these tumors. We have thereby provided highly effective therapies for ovarian cancers.

SEQ ID NO: 1 to SEQ ID NO: 162 correspond to the various polypeptide and polynucleotide sequences set out herein. The database sequence information (including sequences and accession numbers) provided with this description corresponds to the information accessed online as of 12 Feb. 2025.

Any references cited in this specification are hereby incorporated by reference. All amino acid and nucleotide sequences in the references cited in this specification are hereby incorporated into this disclosure. No admission is made that any reference constitutes prior art. Nor does discussion of any reference constitute an admission that such reference forms part of the common general knowledge in the art, in Australia or in any other country.

Persons of ordinary skill can utilise the disclosures and teachings herein to produce other embodiments and variations without undue experimentation. All such embodiments and variations are considered to be encompassed herein.

Accordingly, one of ordinary skill in the art will readily appreciate from the disclosure that later modifications, substitutions, and/or variations performing substantially the same function or achieving substantially the same result as embodiments described herein may be utilized according to such related embodiments. Thus, the present disclosure is intended to encompass, within its scope, the modifications, substitutions, and variations to processes, manufactures, compositions of matter, compounds, means, methods, and/or steps set out herein.

The description herein may contain subject matter that falls outside of the scope of the claimed invention. This subject matter is included to aid understanding of the invention.

What is claimed is:

1. A method of treating a cancer in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein:
(a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUUU, CCUCUGC, UAAGUUAU, UAACUUAU, GUAAAUAG, or CCUCCUGCCUC (SEQ ID NO: 53), wherein the cancer is a nervous system cancer, prostate cancer, kidney cancer, breast cancer, lung cancer, bone cancer, muscle cancer, or eye cancer, and wherein the cancer is characterized by overexpression of MYCN.

2. The method of claim 1, comprising one or more of:
(i) the MYCN gene is amplified in cells of the cancer;
(iia) the cancer is selected from the group consisting of neuroblastoma, medulloblastoma, retinoblastoma, hemangioblastoma, oligodendroglioma, astrocytoma, ependymoma, and glioblastoma multiforme;
(iib) the cancer is characterized by tumors selected from the group consisting of glioma tumors, non-glioma tumors, and rhabdoid tumors;
(iic) the cancer is selected from the group consisting of neuroendocrine prostate cancer, prostate adenocarcinoma, and androgen independent prostate cancer;
(iid) the cancer is characterized as Wilms tumor;
(iie) the cancer is small cell lung cancer;
(iif) the cancer is triple negative breast cancer;
(iig) the cancer is rhabdomyosarcoma or osteosarcoma;
(iii) at least two of the ARE poly(U) stabilizing motifs are substituted;
(iv) at least three of the ARE poly(U) stabilizing motifs are substituted;
(v) at least four of the ARE poly(U) stabilizing motifs are substituted; or
(vi) all of the ARE poly(U) stabilizing motifs are substituted.

3. The method of claim 1, wherein:
(i) the 3′ UTR of the mRNA is at least 80% identical to SEQ ID NO: 3:
CCUCCUGCCUCCAAACAAACAUUGUGUUGACAUUAAGAAUGUUGGUUUA
CUUUCAAAUCGGUCCCCUGUCGAGUUCGGCUCUGGGUGGGCAGUAGGAC
CACCAGUGUGGGGUUCUGCUGGGACCUUGGAGAGCCUGCAUCCCAGGAU
GCUGGGUGGCCCUGCAGCCUCCUCCACCUCACCUCCAUGACAGCGCUAA
ACGUUGGUGACGGUUGGGAGCCUCUGGGGCUGUUGAAGUCACCUUGUGU
GUUCCAAGUUUCCAAACAACAGAAAGUCAUUCCUUCCUCUUAAAAUGGU
GCUUAAGUUCCAGCAGAUGCCACAUAAGGGGUUUGCCAUUUGAUACCCC
UGGGGAACAUUUCUGUAAAUACCAUUGACACAUCCGCCUUUUGUAUACA
UCCUGGGUAAUGAGAGGUGGCUUUUGCGGCCAGUAUUAGACUGGAGUUC
AUACCUAAGUACUGUAAUAAUACCUCAAUGUUUGAGGAGCAUGCCUCGU
AUACAAAUAUAUUGUUAAUCUCUGUUAUGUACUGUACUAAUUCUUACAC
UGCCUGUAUACUUUAGUAUGACGCUGAUACAUAACUAAAUUUGAUACUU
AUAUUUUCGUAUGAAAAUGAGUUGUGAAAGUUUUGAGUAGAUAUUACCU
CAUCACCUCUUGAACUAAGAAACUUUUGUAAAGAAAUUUACUAUAUAUA
UAUGCCUCUGC
(ii) the 3′ UTR of the mRNA is at least 85% identical to SEQ ID NO: 3;
(iii) the 3′ UTR of the mRNA is at least 90% identical to SEQ ID NO: 3;
(iv) the 3′ UTR of the mRNA is at least 950% identical to SEQ ID NO: 3;
(v) the 3′ UTR of the mRNA is at least 99% identical to SEQ ID NO: 3;
(vi) the 3′ UTR of the mRNA comprises SEQ ID NO: 3; or
(vii) the 3′ UTR of the mRNA consists of SEQ ID NO: 3.

4. The method of claim 1, wherein:
(i) the nucleic acid sequence encoding the 3′ UTR is at least 80% identical to SEQ ID NO: 23:
nnnnnnnnnnnnnntcnctcancttcngcctctacacgggcctcgcggc
agggcggtacttcagccattctggtcctgcccgcagatgcgtatnnnng
cttgaatgcaggagctgaacaatcacctatgaattttgcctcgatggca
ttgcgttgannncctcctgcctccaaacaaacattgtgttgacattaag
aatgttggtttactttcaaatcggtcccctgtcgagttcggctctgggt
gggcagtaggaccaccagtgtggggttctgctgggaccttggagagcct
gcatcccaggatgctgggtggccctgcagcctcctccacctcacctcca
tgacagcgctaaacgttggtgacggttgggagcctctggggctgttgaa
gtcaccttgtgtgttccaagtttccaaacaacagaaagtcattccttcc
tcttaaaatggtgcttaagttccagcagatgccacataaggggtttgcc
atttgatacccctggggaacatttctgtaaataccattgacacatccgc
cttttgtatacatcctgggtaatgagaggtggcttttgcggccagtatt
agactggagttcatacctaagtactgtaataatacctcaatgtttgagg
agcatgcctcgtatacaaatatattgttaatctctgttatgtactgta
(ii) the nucleic acid sequence encoding the 3′ UTR is at least 85% identical to SEQ ID NO: 24:
nnnnnnnnnnnnnntcnctcancttcngcctctacacgggcctcgcggc
agggggtacttcagccattctggtcctgcccgcagatgcgtatnnnngc
ttgaatgcaggagctgaacaatcacctatgaattttgcctcgatggcat
tgcgttgannncctcctgcctccaaacaaacattgtgttgacattaaga
atgttggtttactttcaaatcggtcccctgtcgagttcggctctgggtg
ggcagtaggaccaccagtgtggggttctgctgggaccttggagagcctg
catcccaggatgctgggtggccctgcagcctcctccacctcacctccat
gacagcgctaaacgttggtgacggttgggagcctctggggctgttgaag
tcaccttgtgtgttccaagtttccaaacaacagaaagtcattccttcct
cttaaaatggtgcttaagttccagcagatgccacataaggggtttgcca
tttgatacccctggggaacatttctgtaaataccattgacacatccgcc
ttttgtatacatcctgggtaatgagaggtggcttttgcggccagtatta
gactggagttcatacctaagtactgtaataatacctcaatgtttgagga
gcatgcctcgtatacaaatatattgttaatctctgttatgtactgtact
aattcttacactgcctgtatactttagtatgacgctgata
(iii) the nucleic acid sequence encoding the 3′ UTR is at least 90% identical to SEQ ID NO: 25:
nnnnnnnnnnnnnntcnctcancttcngcctctacacgggcctcgcggc
agggcggtacttcagccattctggtcctgcccgcagatgcgtatnnnng
cttgaatgcaggagctgaacaatcacctatgaattttgcctcgatggca
ttgcgttgannncctcctgcctccaaacaaacattgtgttgacattaag
aatgttggtttactttcaaatcggtcccctgtcgagttcggctctgggt
gggcagtaggaccaccagtgtggggttctgctgggaccttggagagcct
gcatcccaggatgctgggtggccctgcagcctcctccacctcacctcca
tgacagcgctaaacgttggtgacggttgggagcctctggggctgttgaa
gtcaccttgtgtgttccaagtttccaaacaacagaaagtcattccttcc
tcttaaaatggtgcttaagttccagcagatgccacataaggggtttgcc
atttgatacccctggggaacatttctgtaaataccattgacacatccgc
cttttgtatacatcctgggtaatgagaggtggcttttgcggccagtatt
agactggagttcatacctaagtactgtaataatacctcaatgtttgagg
agcatgcctcgtatacaaatatattgttaatctctgttatgtactgtac
taattcttacactgcctgtatactttagtatgacgctgatacataacta
aatttgatacttatattttcgtatgaaaatgagtt
(iv) the nucleic acid sequence encoding the 3′ UTR is at least 95% identical to SEQ ID NO: 26:
nnnnnnnnnnnnnntcnctcancttcngcctctacacgggcctcgcggc
agggcggtacttcagccattctggtcctgcccgcagatgcgtatnnnng
cttgaatgcaggagctgaacaatcacctatgaattttgcctcgatggca
ttgcgttgannncctcctgcctccaaacaaacattgtgttgacattaag
aatgttggtttactttcaaatcggtcccctgtcgagttcggctctgggt
gggcagtaggaccaccagtgtggggttctgctgggaccttggagagcct
gcatcccaggatgctgggtggccctgcagcctcctccacctcacctcca
tgacagcgctaaacgttggtgacggttgggagcctctggggctgttgaa
gtcaccttgtgtgttccaagtttccaaacaacagaaagtcattccttcc
tcttaaaatggtgcttaagttccagcagatgccacataaggggtttgcc
atttgatacccctggggaacatttctgtaaataccattgacacatccgc
cttttgtatacatcctgggtaatgagaggtggcttttgcggccagtatt
agactggagttcatacctaagtactgtaataatacctcaatgtttgagg
agcatgcctcgtatacaaatatattgttaatctctgttatgtactgtac
taattcttacactgcctgtatactttagtatgacgctgatacataacta
aatttgatacttatattttcgtatgaaaatgagttgtgaaagttttgag
tagatattacctcatcacctcttgaacta
(v) the nucleic acid sequence encoding the 3′ UTR is at least 99% identical to SEQ ID NO: 188:
nnnnnnnnnnnnnntcnctcancttcngcctctacacgggcctcgcggc
agggcggtacttcagccattctggtcctgcccgcagatgcgtatnnnng
cttgaatgcaggagctgaacaatcacctatgaattttgcctcgatggca
ttgcgttgannncctcctgcctccaaacaaacattgtgttgacattaag
aatgttggtttactttcaaatcggtcccctgtcgagttcggctctgggt
gggcagtaggaccaccagtgtggggttctgctgggaccttggagagcct
gcatcccaggatgctgggtggccctgcagcctcctccacctcacctcca
tgacagcgctaaacgttggtgacggttgggagcctctggggctgttgaa
gtcaccttgtgtgttccaagtttccaaacaacagaaagtcattccttcc
tcttaaaatggtgcttaagttccagcagatgccacataaggggtttgcc
atttgatacccctggggaacatttctgtaaataccattgacacatccgc
cttttgtatacatcctgggtaatgagaggtggcttttgcggccagtatt
agactggagttcatacctaagtactgtaataatacctcaatgtttgagg
agcatgcctcgtatacaaatatattgttaatctctgttatgtactgtac
taattcttacactgcctgtatactttagtatgacgctgatacataacta
aatttgatacttatattttcgtatgaaaatgagttgtgaaagttttgag
tagatattacctcatcacctcttgaactaagaaacttttgtaaagaaat
ttactatatatatatgcctctgc
(vi) the nucleic acid sequence encoding the 3′ UTR comprises SEQ ID NO: 21:
cctcctgcctccaaacaaacattgtgttgacattaagaatgttggttta
ctttcaaatcggtcccctgtcgagttcggctctgggtgggcagtaggac
caccagtgtggggttctgctgggaccttggagagcctgcatcccaggat
gctgggtggccctgcagcctcctccacctcacctccatgacagcgctaa
acgttggtgacggttgggagcctctggggctgttgaagtcaccttgtgt
gttccaagtttccaaacaacagaaagtcattccttcctcttaaaatggt
gcttaagttccagcagatgccacataaggggtttgccatttgatacccc
tggggaacatttctgtaaataccattgacacatccgccttttgtataca
tcctgggtaatgagaggtggcttttgcggccagtattagactggagttc
atacctaagtactgtaataatacctcaatgtttgaggagcatgcctcgt
atacaaatatattgttaatctctgttatgtactgtactaattcttacac
tgcctgtatactttagtatgacgctgatacataactaaatttgatactt
atattttcgtatgaaaatgagttgtgaaagttttgagtagatattacct
catcacctcttgaactaagaaacttttgtaaagaaatttactatatata
tatgcctctgc
or
(vii) the nucleic acid sequence encoding the 3′ UTR consists of SEQ ID NO: 21:
cctcctgcctccaaacaaacattgtgttgacattaagaatgttggttta
ctttcaaatcggtcccctgtcgagttcggctctgggtgggcagtaggac
caccagtgtggggttctgctgggaccttggagagcctgcatcccaggat
gctgggtggccctgcagcctcctccacctcacctccatgacagcgctaa
acgttggtgacggttgggagcctctggggctgttgaagtcaccttgtgt
gttccaagtttccaaacaacagaaagtcattccttcctcttaaaatggt
gcttaagttccagcagatgccacataaggggtttgccatttgatacccc
tggggaacatttctgtaaataccattgacacatccgccttttgtataca
tcctgggtaatgagaggtggcttttgcggccagtattagactggagttc
atacctaagtactgtaataatacctcaatgtttgaggagcatgcctcgt
atacaaatatattgttaatctctgttatgtactgtactaattcttacac
tgcctgtatactttagtatgacgctgatacataactaaatttgatactt
atattttcgtatgaaaatgagttgtgaaagttttgagtagatattacct
catcacctcttgaactaagaaacttttgtaaagaaatttactatatata
tatgcctctgc.

5. The method of claim 1, wherein:
(i) the nucleic acid sequence encoding the 3′ UTR is at least 80% identical to SEQ ID NO: 44:
GCCATCGAGGCAAAATTCCCCCATCTGCGTCAGTCCCCTCAACTTCCGC
CTCTACGCGGGCCTCGCGGCAGGGCGGTACGTCAGCCATTCTGGTCCGC
CGCGCGCATGCGTAGTGGAGCTTGCAGGCTGGAGCTnnnnnnnnnnnnn
ntcnctcancttcngcctctacacgggcctcgcggcagggcggtacttc
agccattctggtcctgcccgcagatgcgtatnnnngcttgaatgcagga
gctgaacaatcacctatgaattttgcctcgatggcattgcgttgannnc
ctcctgcctccaaacaaacattgtgttgacattaagaatgttggtttac
tttcaaatcggtcccctgtcgagttcggctctgggtgggcagtaggacc
accagtgtggggttctgctgggaccttggagagcctgcatcccaggatg
ctgggtggccctgcagcctcctccacctcacctccatgacagcgctaaa
cgttggtgacggttgggagcctctggggctgttgaagtcaccttgtgtg
ttccaagtttccaaacaacagaaagtcattccttcctcttaaaatggtg
cttaagttccagcagatgccacataaggggtttgccatttgatacccct
ggggaacatttctgtaaataccattgacacatccgccttttgtatacat
cctgggtaatgagaggtggcttttgcggccagtattagactggagttca
tacctaagtactgtaataatacctcaatgtttgaggagcatgcctcgta
tacaaatatattgttaatctctgttatgtactgta
(ii) the nucleic acid sequence encoding the 3′ UTR is at least 85% identical to SEQ ID NO: 45:
GCCATCGAGGCAAAATTCCCCCATCTGCGTCAGTCCCCTCAACTTCCGC
CTCTACGCGGGCCTCGCGGCAGGGCGGTACGTCAGCCATTCTGGTCCGC
CGCGCGCATGCGTAGTGGAGCTTGCAGGCTGGAGCTnnnnnnnnnnnnn
ntcnctcancttcngcctctacacgggcctcgcggcagggcggtacttc
agccattctggtcctgcccgcagatgcgtatnnnngcttgaatgcagga
gctgaacaatcacctatgaattttgcctcgatggcattgcgttgannnc
ctcctgcctccaaacaaacattgtgttgacattaagaatgttggtttac
tttcaaatcggtcccctgtcgagttcggctctgggtgggcagtaggacc
accagtgtggggttctgctgggaccttggagagcctgcatcccaggatg
ctgggtggccctgcagcctcctccacctcacctccatgacagcgctaaa
cgttggtgacggttgggagcctctggggctgttgaagtcaccttgtgtg
ttccaagtttccaaacaacagaaagtcattccttcctcttaaaatggtg
cttaagttccagcagatgccacataaggggtttgccatttgatacccct
ggggaacatttctgtaaataccattgacacatccgccttttgtatacat
cctgggtaatgagaggtggcttttgcggccagtattagactggagttca
tacctaagtactgtaataatacctcaatgtttgaggagcatgcctcgta
tacaaatatattgttaatctctgttatgtactgtactaattcttacact
gcctgtatactttagtatgacgctgata
(iii) the nucleic acid sequence encoding the 3′ UTR is at least 90% identical to SEQ ID NO: 46:
GCCATCGAGGCAAAATTCCCCCATCTGCGTCAGTCCCCTCAACTTCCGC
CTCTACGCGGGCCTCGCGGCAGGGCGGTACGTCAGCCATTCTGGTCCGC
CGCGCGCATGCGTAGTGGAGCTTGCAGGCTGGAGCTnnnnnnnnnnnnn
ntcnctcancttcngcctctacacgggcctcgcggcagggcggtacttc
agccattctggtcctgcccgcagatgcgtatnnnngcttgaatgcagga
gctgaacaatcacctatgaattttgcctcgatggcattgcgttgannnc
ctcctgcctccaaacaaacattgtgttgacattaagaatgttggtttac
tttcaaatcggtcccctgtcgagttcggctctgggtgggcagtaggacc
accagtgtggggttctgctgggaccttggagagcctgcatcccaggatg
ctgggggccctgcagcctcctccacctcacctccatgacagcgctaaac
gttggtgacggttgggagcctctggggctgttgaagtcaccttgtgtgt
tccaagtttccaaacaacagaaagtcattccttcctcttaaaatggtgc
ttaagttccagcagatgccacataaggggtttgccatttgatacccctg
gggaacatttctgtaaataccattgacacatccgccttttgtatacatc
ctgggtaatgagaggtggcttttgcggccagtattagactggagttcat
acctaagtactgtaataatacctcaatgtttgaggagcatgcctcgtat
acaaatatattgttaatctctgttatgtactgtactaattcttacactg
cctgtatactttagtatgacgctgatacataactaaatttgatacttat
attttcgtatgaaaatgagtt
(iv) the nucleic acid sequence encoding the 3′ UTR is at least 95% identical to SEQ ID NO: 47:
GCCATCGAGGCAAAATTCCCCCATCTGCGTCAGTCCCCTCAACTTCCGC
CTCTACGCGGGCCTCGCGGCAGGGCGGTACGTCAGCCATTCTGGTCCGC
CGCGCGCATGCGTAGTGGAGCTTGCAGGCTGGAGCTnnnnnnnnnnnnn
ntcnctcancttcngcctctacacgggcctcgcggcagggcggtacttc
agccattctggtcctgcccgcagatgcgtatnnnngcttgaatgcagga
gctgaacaatcacctatgaattttgcctcgatggcattgcgttgannnc
ctcctgcctccaaacaaacattgtgttgacattaagaatgttggtttac
tttcaaatcggtcccctgtcgagttcggctctgggtgggcagtaggacc
accagtgtggggttctgctgggaccttggagagcctgcatcccaggatg
ctgggtggccctgcagcctcctccacctcacctccatgacagcgctaaa
cgttggtgacggttgggagcctctggggctgttgaagtcaccttgtgtg
ttccaagtttccaaacaacagaaagtcattccttcctcttaaaatggtg
cttaagttccagcagatgccacataaggggtttgccatttgatacccct
ggggaacatttctgtaaataccattgacacatccgccttttgtatacat
cctgggtaatgagaggtggcttttgcggccagtattagactggagttca
tacctaagtactgtaataatacctcaatgtttgaggagcatgcctcgta
tacaaatatattgttaatctctgttatgtactgtactaattcttacact
gcctgtatactttagtatgacgctgatacataactaaatttgatactta
tattttcgtatgaaaatgagttgtgaaagttttgagtagatattacctc
atcacctcttgaacta
(v) the nucleic acid sequence encoding the 3′ UTR is at least 99% identical to SEQ ID NO: 48;
GCCATCGAGGCAAAATTCCCCCATCTGCGTCAGTCCCCTCAACTTCCGC
CTCTACGCGGGCCTCGCGGCAGGGCGGTACGTCAGCCATTCTGGTCCGC
CGCGCGCATGCGTAGTGGAGCTTGCAGGCTGGAGCTnnnnnnnnnnnnn
ntcnctcancttcngcctctacacgggcctcgcggcagggcggtacttc
agccattctggtcctgcccgcagatgcgtatnnnngcttgaatgcagga
gctgaacaatcacctatgaattttgcctcgatggcattgcgttgannnc
ctcctgcctccaaacaaacattgtgttgacattaagaatgttggtttac
tttcaaatcggtcccctgtcgagttcggctctgggtgggcagtaggacc
accagtgtggggttctgctgggaccttggagagcctgcatcccaggatg
ctgggtggccctgcagcctcctccacctcacctccatgacagcgctaaa
cgttggtgacggttgggagcctctggggctgttgaagtcaccttgtgtg
ttccaagtttccaaacaacagaaagtcattccttcctcttaaaatggtg
cttaagttccagcagatgccacataaggggtttgccatttgatacccct
ggggaacatttctgtaaataccattgacacatccgccttttgtatacat
cctgggtaatgagaggtggcttttgcggccagtattagactggagttca
tacctaagtactgtaataatacctcaatgtttgaggagcatgcctcgta
tacaaatatattgttaatctctgttatgtactgtactaattcttacact
gcctgtatactttagtatgacgctgatacataactaaatttgatactta
tattttcgtatgaaaatgagttgtgaaagttttgagtagatattacctc
atcacctcttgaactaagaaacttttgtaaagaaatttactatatatat
atgcctctgc
(vi) the nucleic acid sequence encoding the 3′ UTR comprises SEQ ID NO: 43:
GCCATCGAGGCAAAATTCCCCCATCTGCGTCAGTCCCCTCAACTTCCGC
CTCTACGCGGGCCTCGCGGCAGGGCGGTACGTCAGCCATTCTGGTCCGC
CGCGCGCATGCGTAGTGGAGCTTGCAGGCTGGAGCT
cctcctgcctccaaacaaacattgtgttgacattaagaatgttggttta
ctttcaaatcggtcccctgtcgagttcggctctggggggcagtaggacc
accagtgtggggttctgctgggaccttggagagcctgcatcccaggatg
ctgggtggccctgcagcctcctccacctcacctccatgacagcgctaaa
cgttggtgacggttgggagcctctggggctgttgaagtcaccttgtgtg
ttccaagtttccaaacaacagaaagtcattccttcctcttaaaatggtg
cttaagttccagcagatgccacataaggggtttgccatttgatacccct
ggggaacatttctgtaaataccattgacacatccgccttttgtatacat
cctgggtaatgagaggtggcttttgcggccagtattagactggagttca
tacctaagtactgtaataatacctcaatgtttgaggagcatgcctcgta
tacaaatatattgttaatctctgttatgtactgtactaattcttacact
gcctgtatactttagtatgacgctgatacataactaaatttgatactta
tattttcgtatgaaaatgagttgtgaaagttttgagtagatattacctc
atcacctcttgaactaagaaacttttgtaaagaaatttactatatatat
atgcctctgc
or
(vii) the nucleic acid sequence encoding the 3′ UTR consists of SEQ ID NO: 43:
GCCATCGAGGCAAAATTCCCCCATCTGCGTCAGTCCCCTCAACTTCCGC
CTCTACGCGGGCCTCGCGGCAGGGCGGTACGTCAGCCATTCTGGTCCGC
CGCGCGCATGCGTAGTGGAGCTTGCAGGCTGGAGCT
cctcctgcctccaaacaaacattgtgttgacattaagaatgttggttta
ctttcaaatcggtcccctgtcgagttcggctctgggtgggcagtaggac
caccagtgtggggttctgctgggaccttggagagcctgcatcccaggat
gctgggtggccctgcagcctcctccacctcacctccatgacagcgctaa
acgttggtgacggttgggagcctctggggctgttgaagtcaccttgtgt
gttccaagtttccaaacaacagaaagtcattccttcctcttaaaatggt
gcttaagttccagcagatgccacataaggggtttgccatttgatacccc
tggggaacatttctgtaaataccattgacacatccgccttttgtataca
tcctgggtaatgagaggtggcttttgcggccagtattagactggagttc
atacctaagtactgtaataatacctcaatgtttgaggagcatgcctcgt
atacaaatatattgttaatctctgttatgtactgtactaattcttacac
tgcctgtatactttagtatgacgctgatacataactaaatttgatactt
atattttcgtatgaaaatgagttgtgaaagttttgagtagatattacct
catcacctcttgaactaagaaacttttgtaaagaaatttactatatata
tatgcctctgc.

6. A method of treating a cancerous tumor in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein:
(a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUUU, CCUCUGC, UAAGUUAU, UAACUUAU, GUAAAUAG, or CCUCCUGCCUC (SEQ ID NO: 53), wherein the tumor is a nervous system tumor, prostate tumor, kidney tumor, lung tumor, breast tumor, bone tumor, muscle tumor, or eye tumor, wherein the tumor is characterized by overexpression of MYCN, and wherein the tumor is metastatic.

7. The method of claim 6, comprising one or more of:
(i) the MYCN gene is amplified in cells of the tumor;
(iia) the tumor is a neuroblastoma, medulloblastoma, retinoblastoma, hemangioblastoma, oligodendroglioma, astrocytoma, ependymoma, or glioblastoma multiforme tumor;
(iib) the tumor is a glioma tumor, non-glioma tumor, or rhabdoid tumor;
(iic) the tumor is a neuroendocrine prostate cancer, prostate adenocarcinoma, or androgen independent prostate cancer tumor;
(iid) the tumor is a Wilms tumor;
(iie) the tumor is a small cell lung cancer tumor;
(iif) the tumor is a triple negative breast cancer tumor;
(iig) the tumor is a rhabdomyosarcoma tumor or osteosarcoma tumor;
(iii) at least two of the ARE poly(U) stabilizing motifs are substituted;
(iv) at least three of the ARE poly(U) stabilizing motifs are substituted;
(v) at least four of the ARE poly(U) stabilizing motifs are substituted; or
(vi) all of the ARE poly(U) stabilizing motifs are substituted.

8. The method of claim 6, wherein:
(i) the 3′ UTR of the mRNA is at least 80% identical to SEQ ID NO: 3:
CCUCCUGCCUCCAAACAAACAUUGUGUUGACAUUAAGAAUGUUGGUUUACU
UUCAAAUCGGUCCCCUGUCGAGUUCGGCUCUGGGUGGGCAGUAGGACCACC
AGUGUGGGGUUCUGCUGGGACCUUGGAGAGCCUGCAUCCCAGGAUGCUGGG
UGGCCCUGCAGCCUCCUCCACCUCACCUCCAUGACAGCGCUAAACGUUGGU
GACGGUUGGGAGCCUCUGGGGCUGUUGAAGUCACCUUGUGUGUUCCAAGUU
UCCAAACAACAGAAAGUCAUUCCUUCCUCUUAAAAUGGUGCUUAAGUUCCA
GCAGAUGCCACAUAAGGGGUUUGCCAUUUGAUACCCCUGGGGAACAUUUCU
GUAAAUACCAUUGACACAUCCGCCUUUUGUAUACAUCCUGGGUAAUGAGAG
GUGGCUUUUGCGGCCAGUAUUAGACUGGAGUUCAUACCUAAGUACUGUAAU
AAUACCUCAAUGUUUGAGGAGCAUGCCUCGUAUACAAAUAUAUUGUUAAUC
UCUGUUAUGUACUGUACUAAUUCUUACACUGCCUGUAUACUUUAGUAUGAC
GCUGAUACAUAACUAAAUUUGAUACUUAUAUUUUCGUAUGAAAAUGAGUUG
UGAAAGUUUUGAGUAGAUAUUACCUCAUCACCUCUUGAACUAAGAAACUUU
UGUAAAGAAAUUUACUAUAUAUAUAUGCCUCUGC
(ii) the 3′ UTR of the mRNA is at least 85% identical to SEQ ID NO: 3;
(iii) the 3′ UTR of the mRNA is at least 90% identical to SEQ ID NO: 3;
(iv) the 3′ UTR of the mRNA is at least 95% identical to SEQ ID NO: 3;
(v) the 3′ UTR of the mRNA is at least 99% identical to SEQ ID NO: 3;
(vi) the 3′ UTR of the mRNA comprises SEQ ID NO: 3; or
(vii) the 3′ UTR of the mRNA consists of SEQ ID NO: 3.

9. The method of claim 6, wherein:
(i) the nucleic acid sequence encoding the 3′ UTR is at least 80% identical to SEQ ID NO: 23:
nnnnnnnnnnnnnntcnctcancttcngcctctacacgggcctcgcggcag
ggcggtacttcagccattctggtcctgcccgcagatgcgtatnnnngcttg
aatgcaggagctgaacaatcacctatgaattttgcctcgatggcattgcgt
tgannncctcctgcctccaaacaaacattgtgttgacattaagaatgttgg
tttactttcaaatcggtcccctgtcgagttcggctctgggtgggcagtagg
accaccagtgtggggttctgctgggaccttggagagcctgcatcccaggat
gctgggtggccctgcagcctcctccacctcacctccatgacagcgctaaac
gttggtgacggttgggagcctctggggctgttgaagtcaccttgtgtgttc
caagtttccaaacaacagaaagtcattccttcctcttaaaatggtgcttaa
gttccagcagatgccacataaggggtttgccatttgatacccctggggaac
atttctgtaaataccattgacacatccgccttttgtatacatcctgggtaa
tgagaggtggcttttgcggccagtattagactggagttcatacctaagtac
tgtaataatacctcaatgtttgaggagcatgcctcgtatacaaatatattg
ttaatctctgttatgtactgta
(ii) the nucleic acid sequence encoding the 3′ UTR is at least 85% identical to SEQ ID NO: 24:
nnnnnnnnnnnnnntcnctcancttcngcctctacacgggcctcgcggcag
ggggtacttcagccattctggtcctgcccgcagatgcgtatnnnngcttga
atgcaggagctgaacaatcacctatgaattttgcctcgatggcattgcgtt
gannncctcctgcctccaaacaaacattgtgttgacattaagaatgttggt
ttactttcaaatcggtcccctgtcgagttcggctctgggtgggcagtagga
ccaccagtgtggggttctgctgggaccttggagagcctgcatcccaggatg
ctgggtggccctgcagcctcctccacctcacctccatgacagcgctaaacg
ttggtgacggttgggagcctctggggctgttgaagtcaccttgtgtgttcc
aagtttccaaacaacagaaagtcattccttcctcttaaaatggtgcttaag
ttccagcagatgccacataaggggtttgccatttgatacccctggggaaca
tttctgtaaataccattgacacatccgccttttgtatacatcctgggtaat
gagaggtggcttttgcggccagtattagactggagttcatacctaagtact
gtaataatacctcaatgtttgaggagcatgcctcgtatacaaatatattgt
taatctctgttatgtactgtactaattcttacactgcctgtatactttagt
atgacgctgata
(iii) the nucleic acid sequence encoding the 3′ UTR is at least 90% identical to SEQ ID NO: 25:
nnnnnnnnnnnnnntcnctcancttcngcctctacacgggcctcgcggcag
ggcggtacttcagccattctggtcctgcccgcagatgcgtatnnnngcttg
aatgcaggagctgaacaatcacctatgaattttgcctcgatggcattgcgt
tgannncctcctgcctccaaacaaacattgtgttgacattaagaatgttgg
tttactttcaaatcggtcccctgtcgagttcggctctgggtgggcagtagg
accaccagtgtggggttctgctgggaccttggagagcctgcatcccaggat
gctgggtggccctgcagcctcctccacctcacctccatgacagcgctaaac
gttggtgacggttgggagcctctggggctgttgaagtcaccttgtgtgttc
caagtttccaaacaacagaaagtcattccttcctcttaaaatggtgcttaa
gttccagcagatgccacataaggggtttgccatttgatacccctggggaac
atttctgtaaataccattgacacatccgccttttgtatacatcctgggtaa
tgagaggtggcttttgcggccagtattagactggagttcatacctaagtac
tgtaataatacctcaatgtttgaggagcatgcctcgtatacaaatatattg
ttaatctctgttatgtactgtactaattcttacactgcctgtatactttag
tatgacgctgatacataactaaatttgatacttatattttcgtatgaaaat
gagtt
(iv) the nucleic acid sequence encoding the 3′ UTR is at least 95% identical to SEQ ID NO: 26:
nnnnnnnnnnnnnntcnctcancttcngcctctacacgggcctcgcggcag
ggggtacttcagccattctggtcctgcccgcagatgcgtatnnnngcttga
atgcaggagctgaacaatcacctatgaattttgcctcgatggcattgcgtt
gannncctcctgcctccaaacaaacattgtgttgacattaagaatgttggt
ttactttcaaatcggtcccctgtcgagttcggctctgggtgggcagtagga
ccaccagtgtggggttctgctgggaccttggagagcctgcatcccaggatg
ctgggtggccctgcagcctcctccacctcacctccatgacagcgctaaacg
ttggtgacggttgggagcctctggggctgttgaagtcaccttgtgtgttcc
aagtttccaaacaacagaaagtcattccttcctcttaaaatggtgcttaag
ttccagcagatgccacataaggggtttgccatttgatacccctggggaaca
tttctgtaaataccattgacacatccgccttttgtatacatcctgggtaat
gagaggtggcttttgcggccagtattagactggagttcatacctaagtact
gtaataatacctcaatgtttgaggagcatgcctcgtatacaaatatattgt
taatctctgttatgtactgtactaattcttacactgcctgtatactttagt
atgacgctgatacataactaaatttgatacttatattttcgtatgaaaatg
agttgtgaaagttttgagtagatattacctcatcacctcttgaacta
(v) the nucleic acid sequence encoding the 3′ UTR is at least 99% identical to SEQ ID NO: 188:
nnnnnnnnnnnnnntcnctcancttcngcctctacacgggcctcgcggcag
ggcggtacttcagccattctggtcctgcccgcagatgcgtatnnnngcttg
aatgcaggagctgaacaatcacctatgaattttgcctcgatggcattgcgt
tgannncctcctgcctccaaacaaacattgtgttgacattaagaatgttgg
tttactttcaaatcggtcccctgtcgagttcggctctgggtgggcagtagg
accaccagtgtggggttctgctgggaccttggagagcctgcatcccaggat
gctgggtggccctgcagcctcctccacctcacctccatgacagcgctaaac
gttggtgacggttgggagcctctggggctgttgaagtcaccttgtgtgttc
caagtttccaaacaacagaaagtcattccttcctcttaaaatggtgcttaa
gttccagcagatgccacataaggggtttgccatttgatacccctggggaac
atttctgtaaataccattgacacatccgccttttgtatacatcctgggtaa
tgagaggtggcttttgcggccagtattagactggagttcatacctaagtac
tgtaataatacctcaatgtttgaggagcatgcctcgtatacaaatatattg
ttaatctctgttatgtactgtactaattcttacactgcctgtatactttag
tatgacgctgatacataactaaatttgatacttatattttcgtatgaaaat
gagttgtgaaagttttgagtagatattacctcatcacctcttgaactaaga
aacttttgtaaagaaatttactatatatatatgcctctgc
(vi) the nucleic acid sequence encoding the 3′ UTR comprises SEQ ID NO: 21:
cctcctgcctccaaacaaacattgtgttgacattaagaatgttggtttact
ttcaaatcggtcccctgtcgagttcggctctgggtgggcagtaggaccacc
agtgtggggttctgctgggaccttggagagcctgcatcccaggatgctggg
tggccctgcagcctcctccacctcacctccatgacagcgctaaacgttggt
gacggttgggagcctctggggctgttgaagtcaccttgtgtgttccaagtt
tccaaacaacagaaagtcattccttcctcttaaaatggtgcttaagttcca
gcagatgccacataaggggtttgccatttgatacccctggggaacatttct
gtaaataccattgacacatccgccttttgtatacatcctgggtaatgagag
gtggcttttgcggccagtattagactggagttcatacctaagtactgtaat
aatacctcaatgtttgaggagcatgcctcgtatacaaatatattgttaatc
tctgttatgtactgtactaattcttacactgcctgtatactttagtatgac
gctgatacataactaaatttgatacttatattttcgtatgaaaatgagttg
tgaaagttttgagtagatattacctcatcacctcttgaactaagaaacttt
tgtaaagaaatttactatatatatatgcctctgc
or
(vii) the nucleic acid sequence encoding the 3′ UTR consists of SEQ ID NO: 21:
cctcctgcctccaaacaaacattgtgttgacattaagaatgttggtttact
ttcaaatcggtcccctgtcgagttcggctctgggtgggcagtaggaccacc
agtgtggggttctgctgggaccttggagagcctgcatcccaggatgctggg
tggccctgcagcctcctccacctcacctccatgacagcgctaaacgttggt
gacggttgggagcctctggggctgttgaagtcaccttgtgtgttccaagtt
tccaaacaacagaaagtcattccttcctcttaaaatggtgcttaagttcca
gcagatgccacataaggggtttgccatttgatacccctggggaacatttct
gtaaataccattgacacatccgccttttgtatacatcctgggtaatgagag
gtggcttttgcggccagtattagactggagttcatacctaagtactgtaat
aatacctcaatgtttgaggagcatgcctcgtatacaaatatattgttaatc
tctgttatgtactgtactaattcttacactgcctgtatactttagtatgac
gctgatacataactaaatttgatacttatattttcgtatgaaaatgagttg
tgaaagttttgagtagatattacctcatcacctcttgaactaagaaacttt
tgtaaagaaatttactatatatatatgcctctgc.

10. The method of claim 6, wherein:
(i) the nucleic acid sequence encoding the 3′ UTR is at least 80% identical to SEQ ID NO: 44:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttongcctctacacgggcctcgcggcagggggtacttcagcca
ttctggtcctgcccgcagatgcgtatnnnngcttgaatgcaggagctgaa
caatcacctatgaattttgcctcgatggcattgcgttgannncctcctgc
ctccaaacaaacattgtgttgacattaagaatgttggtttactttcaaat
cggtcccctgtcgagttcggctctgggtgggcagtaggaccaccagtgtg
gggttctgctgggaccttggagagcctgcatcccaggatgctgggtggcc
ctgcagcctcctccacctcacctccatgacagcgctaaacgttggtgacg
gttgggagcctctggggctgttgaagtcaccttgtgtgttccaagtttcc
aaacaacagaaagtcattccttcctcttaaaatggtgcttaagttccagc
agatgccacataaggggtttgccatttgatacccctggggaacatttctg
taaataccattgacacatccgccttttgtatacatcctgggtaatgagag
ggtggcttttgcgccagtattagactggagttcatacctaagtactgtaa
taatacctcaatgtttgaggagcatgcctcgtatacaaatatattgttaa
tctctgttatgtactgta
(ii) the nucleic acid sequence encoding the 3′ UTR is at least 85% identifal to SEQ ID NO:45:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggggtacttcagcca
ttctggtcctgcccgcagatgcgtatnnnngcttgaatgcaggagctgaa
caatcacctatgaattttgcctcgatggcattgcgttgannncctcctgc
ctccaaacaaacattgtgttgacattaagaatgttggtttactttcaaat
cggtcccctgtcgagttcggctctgggtgggcagtaggaccaccagtgtg
gggttctgctgggaccttggagagcctgcatcccaggatgctgggtggcc
ctgcagcctcctccacctcacctccatgacagcgctaaacgttggtgacg
gttgggagcctctggggctgttgaagtcaccttgtgtgttccaagtttcc
aaacaacagaaagtcattccttcctcttaaaatggtgcttaagttccagc
agatgccacataaggggtttgccatttgatacccctggggaacatttctg
taaataccattgacacatccgccttttgtatacatcctgggtaatgagag
gtggcttttgcggccagtattagactggagttcatacctaagtactgtaa
taatacctcaatgtttgaggagcatgcctcgtatacaaatatattgttaa
tctctgttatgtactgtactaattcttacactgcctgtatactttagtat
gacgctgata
(iii) the nucleic acid sequence encoding the 3′ UTR is at least 90% identical to SEQ ID NO: 46:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgtactaattcttacactgcctgtatactttagta
tgacgctgatacataactaaatttgatacttatattttcgtatgaaaatg
agtt
(iv) the nucleic acid sequence encoding the 3′ UTR is at least 95% identical to SEQ ID NO: 47:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgtactaattcttacactgcctgtatactttagta
tgacgctgatacataactaaatttgatacttatattttcgtatgaaaatg
agttgtgaaagttttgagtagatattacctcatcacctcttgaacta
(v) the nucleic acid sequence encoding the 3′ UTR is at least 99% identical to SEQ ID NO: 48;
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgtactaattcttacactgcctgtatactttagta
tgacgctgatacataactaaatttgatacttatattttcgtatgaaaatg
agttgtgaaagttttgagtagatattacctcatcacctcttgaactaaga
aacttttgtaaagaaatttactatatatatatgcctctgc
(vi) the nucleic acid sequence encoding the 3′ UTR comprises SEQ ID NO: 43:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCT
cctcctgcctccaaacaaacattgtgttgacattaagaatgttggtttac
tttcaaatcggtcccctgtcgagttcggctctgggtgggcagtaggacca
ccagtgtggggttctgctgggaccttggagagcctgcatcccaggatgct
gggtggccctgcagcctcctccacctcacctccatgacagcgctaaacgt
tggtgacggttgggagcctctggggctgttgaagtcaccttgtgtgttcc
aagtttccaaacaacagaaagtcattccttcctcttaaaatggtgcttaa
gttccagcagatgccacataaggggtttgccatttgatacccctggggaa
catttctgtaaataccattgacacatccgccttttgtatacatcctgggt
aatgagaggtggcttttgcggccagtattagactggagttcatacctaag
tactgtaataatacctcaatgtttgaggagcatgcctcgtatacaaatat
attgttaatctctgttatgtactgtactaattcttacactgcctgtatac
tttagtatgacgctgatacataactaaatttgatacttatattttcgtat
gaaaatgagttgtgaaagttttgagtagatattacctcatcacctcttga
actaagaaacttttgtaaagaaatttactatatatatatgcctctgc
or
(vii) the nucleic acid sequence encoding the 3′ UTR consists of SEQ ID NO: 43:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCT
cctcctgcctccaaacaaacattgtgttgacattaagaatgttggtttac
tttcaaatcggtcccctgtcgagttcggctctgggtgggcagtaggacca
ccagtgtggggttctgctgggaccttggagagcctgcatcccaggatgct
gggtggccctgcagcctcctccacctcacctccatgacagcgctaaacgt
tggtgacggttgggagcctctggggctgttgaagtcaccttgtgtgttcc
aagtttccaaacaacagaaagtcattccttcctcttaaaatggtgcttaa
gttccagcagatgccacataaggggtttgccatttgatacccctggggaa
catttctgtaaataccattgacacatccgccttttgtatacatcctgggt
aatgagaggtggcttttgcggccagtattagactggagttcatacctaag
tactgtaataatacctcaatgtttgaggagcatgcctcgtatacaaatat
attgttaatctctgttatgtactgtactaattcttacactgcctgtatac
tttagtatgacgctgatacataactaaatttgatacttatattttcgtat
gaaaatgagttgtgaaagttttgagtagatattacctcatcacctcttga
actaagaaacttttgtaaagaaatttactatatatatatgcctctgc.

11. A method of preventing metastasis of a cancerous tumor in a subject in need thereof, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein:
(a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUUU, CCUCUGC, UAAGUUAU, UAACUUAU, GUAAAUAG, or CCUCCUGCCUC (SEQ ID NO: 53), wherein the tumor is a nervous system tumor, prostate tumor, kidney tumor, lung tumor, breast tumor, bone tumor, muscle tumor, or eye tumor, and wherein the tumor is characterized by overexpression of MYCN.

12. The method of claim 11, comprising one or more of:
(i) the MYCN gene is amplified in cells of the tumor;
(iia) the tumor is a neuroblastoma, medulloblastoma, retinoblastoma, hemangioblastoma, oligodendroglioma, astrocytoma, ependymoma, or glioblastoma multiforme tumor;
(iib) the tumor is a glioma tumor, non-glioma tumor, or rhabdoid tumor;
(iic) the tumor is a neuroendocrine prostate cancer, prostate adenocarcinoma, or androgen independent prostate cancer tumor;
(iid) the tumor is a Wilms tumor;
(iie) the tumor is a small cell lung cancer tumor;
(iif) the tumor is a triple negative breast cancer tumor;
(iig) the tumor is a rhabdomyosarcoma tumor or osteosarcoma tumor;
(iii) at least two of the ARE poly(U) stabilizing motifs are substituted;
(iv) at least three of the ARE poly(U) stabilizing motifs are substituted;
(v) at least four of the ARE poly(U) stabilizing motifs are substituted; or
(vi) all of the ARE poly(U) stabilizing motifs are substituted.

13. The method of claim 11, wherein:
(i) the 3′ UTR of the mRNA is at least 80% identical to SEQ ID NO: 3:
CCUCCUGCCUCCAAACAAACAUUGUGUUGACAUUAAGAAUGUUGGUUUAC
UUUCAAAUCGGUCCCCUGUCGAGUUCGGCUCUGGGUGGGCAGUAGGACCA
CCAGUGUGGGGUUCUGCUGGGACCUUGGAGAGCCUGCAUCCCAGGAUGCU
GGGUGGCCCUGCAGCCUCCUCCACCUCACCUCCAUGACAGCGCUAAACGU
UGGUGACGGUUGGGAGCCUCUGGGGCUGUUGAAGUCACCUUGUGUGUUCC
AAGUUUCCAAACAACAGAAAGUCAUUCCUUCCUCUUAAAAUGGUGCUUAA
GUUCCAGCAGAUGCCACAUAAGGGGUUUGCCAUUUGAUACCCCUGGGGAA
CAUUUCUGUAAAUACCAUUGACACAUCCGCCUUUUGUAUACAUCCUGGGU
AAUGAGAGGUGGCUUUUGCGGCCAGUAUUAGACUGGAGUUCAUACCUAAG
UACUGUAAUAAUACCUCAAUGUUUGAGGAGCAUGCCUCGUAUACAAAUAU
AUUGUUAAUCUCUGUUAUGUACUGUACUAAUUCUUACACUGCCUGUAUAC
UUUAGUAUGACGCUGAUACAUAACUAAAUUUGAUACUUAUAUUUUCGUAU
GAAAAUGAGUUGUGAAAGUUUUGAGUAGAUAUUACCUCAUCACCUCUUGA
ACUAAGAAACUUUUGUAAAGAAAUUUACUAUAUAUAUAUGCCUCUGC
(ii) the 3′ UTR of the mRNA is at least 85% identical to SEQ ID NO: 3;
(iii) the 3′ UTR of the mRNA is at least 90% identical to SEQ ID NO: 3;
(iv) the 3′ UTR of the mRNA is at least 95% identical to SEQ ID NO: 3;
(v) the 3′ UTR of the mRNA is at least 99% identical to SEQ ID NO: 3;
(vi) the 3′ UTR of the mRNA comprises SEQ ID NO: 3; or
(vii) the 3′ UTR of the mRNA consists of SEQ ID NO: 3.

14. The method of claim 11, wherein:
(i) the nucleic acid sequence encoding the 3′ UTR is at least 80% identical to SEQ ID NO: 23:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctgggtgggca
gtaggaccaccagtgtggggttctgctgggaccttggagagcctgcatcc
caggatgctgggtggccctgcagcctcctccacctcacctccatgacagc
gctaaacgttggtgacggttgggagcctctggggctgttgaagtcacctt
gtgtgttccaagtttccaaacaacagaaagtcattccttcctcttaaaat
ggtgcttaagttccagcagatgccacataaggggtttgccatttgatacc
cctggggaacatttctgtaaataccattgacacatccgccttttgtatac
atcctgggtaatgagaggtggcttttgcggccagtattagactggagttc
atacctaagtactgtaataatacctcaatgtttgaggagcatgcctcgta
tacaaatatattgttaatctctgttatgtactgta
(ii) the nucleic acid sequence encoding the 3′ UTR is at least 85% identical to SEQ ID NO: 24:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctgggtgggca
gtaggaccaccagtgtggggttctgctgggaccttggagagcctgcatcc
caggatgctgggtggccctgcagcctcctccacctcacctccatgacagc
gctaaacgttggtgacggttgggagcctctggggctgttgaagtcacctt
gtgtgttccaagtttccaaacaacagaaagtcattccttcctcttaaaat
ggtgcttaagttccagcagatgccacataaggggtttgccatttgatacc
cctggggaacatttctgtaaataccattgacacatccgccttttgtatac
atcctgggtaatgagaggtggcttttgcggccagtattagactggagttc
atacctaagtactgtaataatacctcaatgtttgaggagcatgcctcgta
tacaaatatattgttaatctctgttatgtactgtactaattcttacactg
cctgtatactttagtatgacgctgata
(iii) the nucleic acid sequence encoding the 3′ UTR is at least 90% identical to SEQ ID NO: 25:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctgggtgggca
gtaggaccaccagtgtggggttctgctgggaccttggagagcctgcatcc
caggatgctgggtggccctgcagcctcctccacctcacctccatgacagc
gctaaacgttggtgacggttgggagcctctggggctgttgaagtcacctt
gtgtgttccaagtttccaaacaacagaaagtcattccttcctcttaaaat
ggtgcttaagttccagcagatgccacataaggggtttgccatttgatacc
cctggggaacatttctgtaaataccattgacacatccgccttttgtatac
atcctgggtaatgagaggtggcttttgcggccagtattagactggagttc
atacctaagtactgtaataatacctcaatgtttgaggagcatgcctcgta
tacaaatatattgttaatctctgttatgtactgtactaattcttacactg
cctgtatactttagtatgacgctgatacataactaaatttgatacttata
ttttcgtatgaaaatgagtt
(iv) the nucleic acid sequence encoding the 3′ UTR is at least 95% identical to SEQ ID NO: 26:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctggggggcag
taggaccaccagtgtggggttctgctgggaccttggagagcctgcatccc
aggatgctgggtggccctgcagcctcctccacctcacctccatgacagcg
ctaaacgttggtgacggttgggagcctctggggctgttgaagtcaccttg
tgtgttccaagtttccaaacaacagaaagtcattccttcctcttaaaatg
gtgcttaagttccagcagatgccacataaggggtttgccatttgataccc
ctggggaacatttctgtaaataccattgacacatccgccttttgtataca
tcctgggtaatgagaggtggcttttgcggccagtattagactggagttca
tacctaagtactgtaataatacctcaatgtttgaggagcatgcctcgtat
acaaatatattgttaatctctgttatgtactgtactaattcttacactgc
ctgtatactttagtatgacgctgatacataactaaatttgatacttatat
tttcgtatgaaaatgagttgtgaaagttttgagtagatattacctcatca
cctcttgaacta
(v) the nucleic acid sequence encoding the 3′ UTR is at least 99% identical to SEQ ID NO: 188:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctgggtgggca
gtaggaccaccagtgtggggttctgctgggaccttggagagcctgcatcc
caggatgctgggtggccctgcagcctcctccacctcacctccatgacagc
gctaaacgttggtgacggttgggagcctctggggctgttgaagtcacctt
gtgtgttccaagtttccaaacaacagaaagtcattccttcctcttaaaat
ggtgcttaagttccagcagatgccacataaggggtttgccatttgatacc
cctggggaacatttctgtaaataccattgacacatccgccttttgtatac
atcctgggtaatgagaggtggcttttgcggccagtattagactggagttc
atacctaagtactgtaataatacctcaatgtttgaggagcatgcctcgta
tacaaatatattgttaatctctgttatgtactgtactaattcttacactg
cctgtatactttagtatgacgctgatacataactaaatttgatacttata
ttttcgtatgaaaatgagttgtgaaagttttgagtagatattacctcatc
acctcttgaactaagaaacttttgtaaagaaatttactatatatatatgc
ctctgc
(vi) the nucleic acid sequence encoding the 3′ UTR comprises SEQ ID NO: 21:
cctcctgcctccaaacaaacattgtgttgacattaagaatgttggtttac
tttcaaatcggtcccctgtcgagttcggctctgggtgggcagtaggacca
ccagtgtggggttctgctgggaccttggagagcctgcatcccaggatgct
gggtggccctgcagcctcctccacctcacctccatgacagcgctaaacgt
tggtgacggttgggagcctctggggctgttgaagtcaccttgtgtgttcc
aagtttccaaacaacagaaagtcattccttcctcttaaaatggtgcttaa
gttccagcagatgccacataaggggtttgccatttgatacccctggggaa
catttctgtaaataccattgacacatccgccttttgtatacatcctgggt
aatgagaggtggcttttgcggccagtattagactggagttcatacctaag
tactgtaataatacctcaatgtttgaggagcatgcctcgtatacaaatat
attgttaatctctgttatgtactgtactaattcttacactgcctgtatac
tttagtatgacgctgatacataactaaatttgatacttatattttcgtat
gaaaatgagttgtgaaagttttgagtagatattacctcatcacctcttga
actaagaaacttttgtaaagaaatttactatatatatatgcctctgc
or
(vii) the nucleic acid sequence encoding the 3′ UTR consists of SEQ ID NO: 21:
cctcctgcctccaaacaaacattgtgttgacattaagaatgttggtttac
tttcaaatcggtcccctgtcgagttcggctctgggtgggcagtaggacca
ccagtgtggggttctgctgggaccttggagagcctgcatcccaggatgct
gggtggccctgcagcctcctccacctcacctccatgacagcgctaaacgt
tggtgacggttgggagcctctggggctgttgaagtcaccttgtgtgttcc
aagtttccaaacaacagaaagtcattccttcctcttaaaatggtgcttaa
gttccagcagatgccacataaggggtttgccatttgatacccctggggaa
catttctgtaaataccattgacacatccgccttttgtatacatcctgggt
aatgagaggtggcttttgcggccagtattagactggagttcatacctaag
tactgtaataatacctcaatgtttgaggagcatgcctcgtatacaaatat
attgttaatctctgttatgtactgtactaattcttacactgcctgtatac
tttagtatgacgctgatacataactaaatttgatacttatattttcgtat
gaaaatgagttgtgaaagttttgagtagatattacctcatcacctcttga
actaagaaacttttgtaaagaaatttactatatatatatgcctctgc.

15. The method of claim 11, wherein:
(i) the nucleic acid sequence encoding the 3′ UTR is at least 80% identical to SEQ ID NO: 44:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggggtacttcagcca
ttctggtcctgcccgcagatgcgtatnnnngcttgaatgcaggagctgaa
caatcacctatgaattttgcctcgatggcattgcgttgannncctcctgc
ctccaaacaaacattgtgttgacattaagaatgttggtttactttcaaat
cggtcccctgtcgagttcggctctgggtgggcagtaggaccaccagtgtg
gggttctgctgggaccttggagagcctgcatcccaggatgctgggtggcc
ctgcagcctcctccacctcacctccatgacagcgctaaacgttggtgacg
gttgggagcctctggggctgttgaagtcaccttgtgtgttccaagtttcc
aaacaacagaaagtcattccttcctcttaaaatggtgcttaagttccagc
agatgccacataaggggtttgccatttgatacccctggggaacatttctg
taaataccattgacacatccgccttttgtatacatcctgggtaatgagag
gtggcttttgcggccagtattagactggagttcatacctaagtactgtaa
taatacctcaatgtttgaggagcatgcctcgtatacaaatatattgttaa
tctctgttatgtactgta
(ii) the nucleic acid sequence encoding the 3′ UTR is at least 85% identical to SEQ ID NO: 45:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgtactaattcttacactgcctgtatactttagta
tgacgctgata
(iii) the nucleic acid sequence encoding the 3′ UTR is at least 90% identical to SEQ ID NO: 46:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgtactaattcttacactgcctgtatactttagta
tgacgctgatacataactaaatttgatacttatattttcgtatgaaaatg
agtt
(iv) the nucleic acid sequence encoding the 3′ UTR is at least 95% identical to SEQ ID NO: 47:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgtactaattcttacactgcctgtatactttagta
tgacgctgatacataactaaatttgatacttatattttcgtatgaaaatg
agttgtgaaagttttgagtagatattacctcatcacctcttgaacta
(v) the nucleic acid sequence encoding the 3′ UTR is at least 99% identical to SEQ ID NO: 48;
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgtactaattcttacactgcctgtatactttagta
tgacgctgatacataactaaatttgatacttatattttcgtatgaaaatg
agttgtgaaagttttgagtagatattacctcatcacctcttgaactaaga
aacttttgtaaagaaatttactatatatatatgcctctgc
(vi) the nucleic acid sequence encoding the 3′ UTR comprises SEQ ID NO: 43:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTcctcctgcctccaaac
aaacattgtgttgacattaagaatgttggtttactttcaaatcggtcccc
tgtcgagttcggctctgggtgggcagtaggaccaccagtgtggggttctg
ctgggaccttggagagcctgcatcccaggatgctgggtggccctgcagcc
tcctccacctcacctccatgacagcgctaaacgttggtgacggttgggag
cctctggggctgttgaagtcaccttgtgtgttccaagtttccaaacaaca
gaaagtcattccttcctcttaaaatggtgcttaagttccagcagatgcca
cataaggggtttgccatttgatacccctggggaacatttctgtaaatacc
attgacacatccgccttttgtatacatcctgggtaatgagaggtggcttt
tgcggccagtattagactggagttcatacctaagtactgtaataatacct
caatgtttgaggagcatgcctcgtatacaaatatattgttaatctctgtt
atgtactgtactaattcttacactgcctgtatactttagtatgacgctga
tacataactaaatttgatacttatattttcgtatgaaaatgagttgtgaa
agttttgagtagatattacctcatcacctcttgaactaagaaacttttgt
aaagaaatttactatatatatatgcctctgc
or
(vii) the nucleic acid sequence encoding the 3′ UTR consists of SEQ ID NO: 43:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTcctcctgcctccaaac
aaacattgtgttgacattaagaatgttggtttactttcaaatcggtcccc
tgtcgagttcggctctgggtgggcagtaggaccaccagtgtggggttctg
ctgggaccttggagagcctgcatcccaggatgctgggtggccctgcagcc
tcctccacctcacctccatgacagcgctaaacgttggtgacggttgggag
cctctggggctgttgaagtcaccttgtgtgttccaagtttccaaacaaca
gaaagtcattccttcctcttaaaatggtgcttaagttccagcagatgcca
cataaggggtttgccatttgatacccctggggaacatttctgtaaatacc
attgacacatccgccttttgtatacatcctgggtaatgagaggtggcttt
tgcggccagtattagactggagttcatacctaagtactgtaataatacct
caatgtttgaggagcatgcctcgtatacaaatatattgttaatctctgtt
atgtactgtactaattcttacactgcctgtatactttagtatgacgctga
tacataactaaatttgatacttatattttcgtatgaaaatgagttgtgaa
agttttgagtagatattacctcatcacctcttgaactaagaaacttttgt
aaagaaatttactatatatatatgcctctgc.

16. A method of treating a cancer in a subject having cancer progression after chemotherapy treatment, the method comprising administering to the subject a pharmaceutical composition comprising a plasmid vector comprising a therapeutically effective amount of a DNA molecule, wherein the DNA molecule comprises in the 5′ to 3′ direction of transcription relative to synthesis of a messenger RNA (mRNA) molecule comprising a 3′ UTR of an mRNA encoding a MYCN protein:
(a) a promoter, operatively linked to: (b) a nucleic acid sequence encoding a 3′ UTR of a MYCN gene in the mRNA molecule in which one or more ARE poly(U) stabilizing motifs of the 3′ UTR of the mRNA are substituted with a nucleic acid sequence selected from CCUC, CUGC, ACCUC, AUUUU, CCUCUGC, UAAGUUAU, UAACUUAU, GUAAAUAG, or CCUCCUGCCUC (SEQ ID NO: 53), wherein the cancer is a nervous system cancer, prostate cancer, kidney cancer, lung cancer, breast cancer, bone cancer, muscle cancer, or eye cancer, and wherein the cancer is characterized by overexpression of MYCN.

17. The method of claim 16, comprising one or more of:
(i) the MYCN gene is amplified in cells of the cancer;
(iia) the cancer is selected from the group consisting of neuroblastoma, medulloblastoma, retinoblastoma, hemangioblastoma, oligodendroglioma, astrocytoma, ependymoma, and glioblastoma multiforme;
(iib) the cancer is characterized by tumors selected from the group consisting of glioma tumors, non-glioma tumors, and rhabdoid tumors;
(iic) the cancer is selected from the group consisting of neuroendocrine prostate cancer, prostate adenocarcinoma, and androgen independent prostate cancer;
(iid) the cancer is characterized as Wilms tumor;
(iie) the cancer is small cell lung cancer;
(iif) the cancer is triple negative breast cancer;
(iig) the cancer is rhabdomyosarcoma or osteosarcoma;
(iii) at least two of the ARE poly(U) stabilizing motifs are substituted;
(iv) at least three of the ARE poly(U) stabilizing motifs are substituted;
(v) at least four of the ARE poly(U) stabilizing motifs are substituted; or
(vi) all of the ARE poly(U) stabilizing motifs are substituted.

18. The method of claim 16 or claim 17, wherein:
(i) the 3′ UTR of the mRNA is at least 80% identical to SEQ ID NO: 3:
CCUCCUGCCUCCAAACAAACAUUGUGUUGACAUUAAGAAUGUUGGUUUACU
UUCAAAUCGGUCCCCUGUCGAGUUCGGCUCUGGGUGGGCAGUAGGACCACC
AGUGUGGGGUUCUGCUGGGACCUUGGAGAGCCUGCAUCCCAGGAUGCUGGG
UGGCCCUGCAGCCUCCUCCACCUCACCUCCAUGACAGCGCUAAACGUUGGU
GACGGUUGGGAGCCUCUGGGGCUGUUGAAGUCACCUUGUGUGUUCCAAGUU
UCCAAACAACAGAAAGUCAUUCCUUCCUCUUAAAAUGGUGCUUAAGUUCCA
GCAGAUGCCACAUAAGGGGUUUGCCAUUUGAUACCCCUGGGGAACAUUUCU
GUAAAUACCAUUGACACAUCCGCCUUUUGUAUACAUCCUGGGUAAUGAGAG
GUGGCUUUUGCGGCCAGUAUUAGACUGGAGUUCAUACCUAAGUACUGUAAU
AAUACCUCAAUGUUUGAGGAGCAUGCCUCGUAUACAAAUAUAUUGUUAAUC
UCUGUUAUGUACUGUACUAAUUCUUACACUGCCUGUAUACUUUAGUAUGAC
GCUGAUACAUAACUAAAUUUGAUACUUAUAUUUUCGUAUGAAAAUGAGUUG
UGAAAGUUUUGAGUAGAUAUUACCUCAUCACCUCUUGAACUAAGAAACUUU
UGUAAAGAAAUUUACUAUAUAUAUAUGCCUCUGC
(ii) the 3′ UTR of the mRNA is at least 85% identical to SEQ ID NO: 3;
(iii) the 3′ UTR of the mRNA is at least 90% identical to SEQ ID NO: 3;
(iv) the 3′ UTR of the mRNA is at least 95% identical to SEQ ID NO: 3;
(v) the 3′ UTR of the mRNA is at least 99% identical to SEQ ID NO: 3;
(vi) the 3′ UTR of the mRNA comprises SEQ ID NO: 3; or
(vii) the 3′ UTR of the mRNA consists of SEQ ID NO: 3.

19. The method of claim 16, wherein:
(i) the nucleic acid sequence encoding the 3′ UTR is at least 80% identical to SEQ ID NO: 23:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctgggtgggca
gtaggaccaccagtgtggggttctgctgggaccttggagagcctgcatcc
caggatgctgggtggccctgcagcctcctccacctcacctccatgacagc
gctaaacgttggtgacggttgggagcctctggggctgttgaagtcacctt
gtgtgttccaagtttccaaacaacagaaagtcattccttcctcttaaaat
ggtgcttaagttccagcagatgccacataaggggtttgccatttgatacc
cctggggaacatttctgtaaataccattgacacatccgccttttgtatac
atcctgggtaatgagaggtggcttttgcggccagtattagactggagttc
atacctaagtactgtaataatacctcaatgtttgaggagcatgcctcgta
tacaaatatattgttaatctctgttatgtactgta
(ii) the nucleic acid sequence encoding the 3′ UTR is at least 85% identical to SEQ ID NO: 24:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctgggtgggca
gtaggaccaccagtgtggggttctgctgggaccttggagagcctgcatcc
caggatgctgggtggccctgcagcctcctccacctcacctccatgacagc
gctaaacgttggtgacggttgggagcctctggggctgttgaagtcacctt
gtgtgttccaagtttccaaacaacagaaagtcattccttcctcttaaaat
ggtgcttaagttccagcagatgccacataaggggtttgccatttgatacc
cctggggaacatttctgtaaataccattgacacatccgccttttgtatac
atcctgggtaatgagaggtggcttttgcggccagtattagactggagttc
atacctaagtactgtaataatacctcaatgtttgaggagcatgcctcgta
tacaaatatattgttaatctctgttatgtactgtactaattcttacactg
cctgtatactttagtatgacgctgata
(iii) the nucleic acid sequence encoding the 3′ UTR is at least 90% identical to SEQ ID NO: 25:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctgggtgggca
gtaggaccaccagtgtggggttctgctgggaccttggagagcctgcatcc
caggatgctgggtggccctgcagcctcctccacctcacctccatgacagc
gctaaacgttggtgacggttgggagcctctggggctgttgaagtcacctt
gtgtgttccaagtttccaaacaacagaaagtcattccttcctcttaaaat
ggtgcttaagttccagcagatgccacataaggggtttgccatttgatacc
cctggggaacatttctgtaaataccattgacacatccgccttttgtatac
atcctgggtaatgagaggtggcttttgcggccagtattagactggagttc
atacctaagtactgtaataatacctcaatgtttgaggagcatgcctcgta
tacaaatatattgttaatctctgttatgtactgtactaattcttacactg
cctgtatactttagtatgacgctgatacataactaaatttgatacttata
ttttcgtatgaaaatgagtt
(iv) the nucleic acid sequence encoding the 3′ UTR is at least 95% identical to SEQ ID NO: 26:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctgggtgggca
gtaggaccaccagtgtggggttctgctgggaccttggagagcctgcatcc
caggatgctgggtggccctgcagcctcctccacctcacctccatgacagc
gctaaacgttggtgacggttgggagcctctggggctgttgaagtcacctt
gtgtgttccaagtttccaaacaacagaaagtcattccttcctcttaaaat
ggtgcttaagttccagcagatgccacataaggggtttgccatttgatacc
cctggggaacatttctgtaaataccattgacacatccgccttttgtatac
atcctgggtaatgagaggtggcttttgcggccagtattagactggagttc
atacctaagtactgtaataatacctcaatgtttgaggagcatgcctcgta
tacaaatatattgttaatctctgttatgtactgtactaattcttacactg
cctgtatactttagtatgacgctgatacataactaaatttgatacttata
ttttcgtatgaaaatgagttgtgaaagttttgagtagatattacctcatc
acctcttgaacta
(v) the nucleic acid sequence encoding the 3′ UTR is at least 99% identical to SEQ ID NO: 188:
nnnnnnnnnnnnnntcnctcancttcngcctctacacgggcctcgcggca
gggcggtacttcagccattctggtcctgcccgcagatgcgtatnnnngct
tgaatgcaggagctgaacaatcacctatgaattttgcctcgatggcattg
cgttgannncctcctgcctccaaacaaacattgtgttgacattaagaatg
ttggtttactttcaaatcggtcccctgtcgagttcggctctggggggcag
taggaccaccagtgtggggttctgctgggaccttggagagcctgcatccc
aggatgctgggtggccctgcagcctcctccacctcacctccatgacagcg
ctaaacgttggtgacggttgggagcctctggggctgttgaagtcaccttg
tgtgttccaagtttccaaacaacagaaagtcattccttcctcttaaaatg
gtgcttaagttccagcagatgccacataaggggtttgccatttgataccc
ctggggaacatttctgtaaataccattgacacatccgccttttgtataca
tcctgggtaatgagaggtggcttttgcggccagtattagactggagttca
tacctaagtactgtaataatacctcaatgtttgaggagcatgcctcgtat
acaaatatattgttaatctctgttatgtactgtactaattcttacactgc
ctgtatactttagtatgacgctgatacataactaaatttgatacttatat
tttcgtatgaaaatgagttgtgaaagttttgagtagatattacctcatca
cctcttgaactaagaaacttttgtaaagaaatttactatatatatatgcc
tctgc
(vi) the nucleic acid sequence encoding the 3′ UTR comprises SEQ ID NO: 21:
cctcctgcctccaaacaaacattgtgttgacattaagaatgttggtttac
tttcaaatcggtcccctgtcgagttcggctctgggtgggcagtaggacca
ccagtgtggggttctgctgggaccttggagagcctgcatcccaggatgct
gggtggccctgcagcctcctccacctcacctccatgacagcgctaaacgt
tggtgacggttgggagcctctggggctgttgaagtcaccttgtgtgttcc
aagtttccaaacaacagaaagtcattccttcctcttaaaatggtgcttaa
gttccagcagatgccacataaggggtttgccatttgatacccctggggaa
catttctgtaaataccattgacacatccgccttttgtatacatcctgggt
aatgagaggtggcttttgcggccagtattagactggagttcatacctaag
tactgtaataatacctcaatgtttgaggagcatgcctcgtatacaaatat
attgttaatctctgttatgtactgtactaattcttacactgcctgtatac
tttagtatgacgctgatacataactaaatttgatacttatattttcgtat
gaaaatgagttgtgaaagttttgagtagatattacctcatcacctcttga
actaagaaacttttgtaaagaaatttactatatatatatgcctctgc
or
(vii) the nucleic acid sequence encoding the 3′ UTR consists of SEQ ID NO: 21:
cctcctgcctccaaacaaacattgtgttgacattaagaatgttggtttac
tttcaaatcggtcccctgtcgagttcggctctgggtgggcagtaggacca
ccagtgtggggttctgctgggaccttggagagcctgcatcccaggatgct
gggtggccctgcagcctcctccacctcacctccatgacagcgctaaacgt
tggtgacggttgggagcctctggggctgttgaagtcaccttgtgtgttcc
aagtttccaaacaacagaaagtcattccttcctcttaaaatggtgcttaa
gttccagcagatgccacataaggggtttgccatttgatacccctggggaa
catttctgtaaataccattgacacatccgccttttgtatacatcctgggt
aatgagaggtggcttttgcggccagtattagactggagttcatacctaag
tactgtaataatacctcaatgtttgaggagcatgcctcgtatacaaatat
attgttaatctctgttatgtactgtactaattcttacactgcctgtatac
tttagtatgacgctgatacataactaaatttgatacttatattttcgtat
gaaaatgagttgtgaaagttttgagtagatattacctcatcacctcttga
actaagaaacttttgtaaagaaatttactatatatatatgcctctgc.

20. The method of claim 16, wherein:
(i) the nucleic acid sequence encoding the 3′ UTR is at least 80% identical to SEQ ID NO: 44:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgta
(ii) the nucleic acid sequence encoding the 3′ UTR is at least 85% identical to SEQ ID NO: 45:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgtactaattcttacactgcctgtatactttagta
tgacgctgata
(iii) the nucleic acid sequence encoding the 3′ UTR is at least 90% identical to SEQ ID NO: 46:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggggtacttcagcca
ttctggtcctgcccgcagatgcgtatnnnngcttgaatgcaggagctgaa
caatcacctatgaattttgcctcgatggcattgcgttgannncctcctgc
ctccaaacaaacattgtgttgacattaagaatgttggtttactttcaaat
cggtcccctgtcgagttcggctctgggtgggcagtaggaccaccagtgtg
gggttctgctgggaccttggagagcctgcatcccaggatgctgggtggcc
ctgcagcctcctccacctcacctccatgacagcgctaaacgttggtgacg
gttgggagcctctggggctgttgaagtcaccttgtgtgttccaagtttcc
aaacaacagaaagtcattccttcctcttaaaatggtgcttaagttccagc
agatgccacataaggggtttgccatttgatacccctggggaacatttctg
taaataccattgacacatccgccttttgtatacatcctgggtaatgagag
gtggcttttgcggccagtattagactggagttcatacctaagtactgtaa
taatacctcaatgtttgaggagcatgcctcgtatacaaatatattgttaa
tctctgttatgtactgtactaattcttacactgcctgtatactttagtat
gacgctgatacataactaaatttgatacttatattttcgtatgaaaatga
gtt
(iv) the nucleic acid sequence encoding the 3′ UTR is at least 95% identical to SEQ ID NO: 47:
GCCATCGAGGCAAAATTCCCCCATCTGCGTCAGTCCCCTCAACTTCCGC
CTCTACGCGGGCCTCGCGGCAGGGCGGTACGTCAGCCATTCTGGTCCGC
CGCGCGCATGCGTAGTGGAGCTTGCAGGCTGGAGCTnnnnnnnnnnnnn
ntcnctcancttcngcctctacacgggcctcgcggcagggcggtacttc
agccattctggtcctgcccgcagatgcgtatnnnngcttgaatgcagga
gctgaacaatcacctatgaattttgcctcgatggcattgcgttgannnc
ctcctgcctccaaacaaacattgtgttgacattaagaatgttggtttac
tttcaaatcggtcccctgtcgagttcggctctgggtgggcagtaggacc
accagtgtggggttctgctgggaccttggagagcctgcatcccaggatg
ctgggtggccctgcagcctcctccacctcacctccatgacagcgctaaa
cgttggtgacggttgggagcctctggggctgttgaagtcaccttgtgtg
ttccaagtttccaaacaacagaaagtcattccttcctcttaaaatggtg
cttaagttccagcagatgccacataaggggtttgccatttgatacccct
ggggaacatttctgtaaataccattgacacatccgccttttgtatacat
cctgggtaatgagaggtggcttttgcggccagtattagactggagttca
tacctaagtactgtaataatacctcaatgtttgaggagcatgcctcgta
tacaaatatattgttaatctctgttatgtactgtactaattcttacact
gcctgtatactttagtatgacgctgatacataactaaatttgatactta
tattttcgtatgaaaatgagttgtgaaagttttgagtagatattacctc
atcacctcttgaacta
(v) the nucleic acid sequence encoding the 3′ UTR is at least 99% identical to SEQ ID NO: 48;
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTnnnnnnnnnnnnnntc
nctcancttcngcctctacacgggcctcgcggcagggcggtacttcagcc
attctggtcctgcccgcagatgcgtatnnnngcttgaatgcaggagctga
acaatcacctatgaattttgcctcgatggcattgcgttgannncctcctg
cctccaaacaaacattgtgttgacattaagaatgttggtttactttcaaa
tcggtcccctgtcgagttcggctctgggtgggcagtaggaccaccagtgt
ggggttctgctgggaccttggagagcctgcatcccaggatgctgggtggc
cctgcagcctcctccacctcacctccatgacagcgctaaacgttggtgac
ggttgggagcctctggggctgttgaagtcaccttgtgtgttccaagtttc
caaacaacagaaagtcattccttcctcttaaaatggtgcttaagttccag
cagatgccacataaggggtttgccatttgatacccctggggaacatttct
gtaaataccattgacacatccgccttttgtatacatcctgggtaatgaga
ggtggcttttgcggccagtattagactggagttcatacctaagtactgta
ataatacctcaatgtttgaggagcatgcctcgtatacaaatatattgtta
atctctgttatgtactgtactaattcttacactgcctgtatactttagta
tgacgctgatacataactaaatttgatacttatattttcgtatgaaaatg
agttgtgaaagttttgagtagatattacctcatcacctcttgaactaaga
aacttttgtaaagaaatttactatatatatatgcctctgc
(vi) the nucleic acid sequence encoding the 3′ UTR comprises SEQ ID NO: 43:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTcctcctgcctccaaac
aaacattgtgttgacattaagaatgttggtttactttcaaatcggtcccc
tgtcgagttcggctctgggtgggcagtaggaccaccagtgtggggttctg
ctgggaccttggagagcctgcatcccaggatgctgggtggccctgcagcc
tcctccacctcacctccatgacagcgctaaacgttggtgacggttgggag
cctctggggctgttgaagtcaccttgtgtgttccaagtttccaaacaaca
gaaagtcattccttcctcttaaaatggtgcttaagttccagcagatgcca
cataaggggtttgccatttgatacccctggggaacatttctgtaaatacc
attgacacatccgccttttgtatacatcctgggtaatgagaggtggcttt
tgcggccagtattagactggagttcatacctaagtactgtaataatacct
caatgtttgaggagcatgcctcgtatacaaatatattgttaatctctgtt
atgtactgtactaattcttacactgcctgtatactttagtatgacgctga
tacataactaaatttgatacttatattttcgtatgaaaatgagttgtgaa
agttttgagtagatattacctcatcacctcttgaactaagaaacttttgt
aaagaaatttactatatatatatgcctctgc
or
(vii) the nucleic acid sequence encoding the 3′ UTR consists of SEQ ID NO: 43:
GCCATCGAGGCAAAATTCCCCCATCTGCGTCAGTCCCCTCAACTTCCGCC
TCTACGCGGGCCTCGCGGCAGGGCGGTACGTCAGCCATTCTGGTCCGCCG
CGCGCATGCGTAGTGGAGCTTGCAGGCTGGAGCTcctcctgcctccaaac
aaacattgtgttgacattaagaatgttggtttactttcaaatcggtcccc
tgtcgagttcggctctgggtgggcagtaggaccaccagtgtggggttctg
ctgggaccttggagagcctgcatcccaggatgctgggtggccctgcagcc
tcctccacctcacctccatgacagcgctaaacgttggtgacggttgggag
cctctggggctgttgaagtcaccttgtgtgttccaagtttccaaacaaca
gaaagtcattccttcctcttaaaatggtgcttaagttccagcagatgcca
cataaggggtttgccatttgatacccctggggaacatttctgtaaatacc
attgacacatccgccttttgtatacatcctgggtaatgagaggtggcttt
tgcggccagtattagactggagttcatacctaagtactgtaataatacct
caatgtttgaggagcatgcctcgtatacaaatatattgttaatctctgtt
atgtactgtactaattcttacactgcctgtatactttagtatgacgctga
tacataactaaatttgatacttatattttcgtatgaaaatgagttgtgaa
agttttgagtagatattacctcatcacctcttgaactaagaaacttttgt
aaagaaatttactatatatatatgcctctgc.
